(19) United States
(12) Patent Application Publication
(10) Pub. No.: US 2024/0034806 A1
(43) Pub. Date: Feb. 1, 2024

(54) COMPOSITIONS AND METHODS RELATED TO TUMOR ACTIVATED ANTIBODIES TARGETING TROP2 AND EFFECTOR CELL ANTIGENS

(71) Applicant: Janux Therapeutics, Inc., San Diego, CA (US)

(72) Inventors: David CAMPBELL, San Diego, CA (US); Thomas R. DIRAIMONDO, San Diego, CA (US)

(21) Appl. No.: 18/256,271

(22) PCT Filed: Dec. 7, 2021
(86) PCT No.: PCT/US2021/062261

§ 371 Date: Jun. 7, 2023

Related U.S. Application Data
(60) Provisional application No. 63/187,719, filed on May 12, 2021.
(60) Provisional application No. 63/123,327, filed on Dec. 9, 2020.

(51) Int. Cl. C07K 16/30; A61P 35/00; C07K 16/28

(52) U.S. Cl. CPC C07K 16/30; A61P 35/00; C07K 16/2809; C07K 2319/50; C07K 2317/92; C07K 2317/622; C07K 2317/94; C07K 2317/31; C07K 2317/73; C07K 2317/55

(57) ABSTRACT

Provided herein are multispecific antibodies that selectively bind to TROP2 and effector cell antigens such as CD3, pharmaceutical compositions thereof, as well as nucleic acids, and methods for making and discovering the same.

### CROSS-REFERENCE

The present application claims the benefit of U.S. Provisional Application No. 63/123,327, filed Dec. 9, 2020, U.S. Provisional Application No. 63/187,719, filed May 12, 2021, each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Nov. 29, 2021, is named 52426-729_601_SL.txt and is 934,933 bytes in size.

### SUMMARY

Disclosed herein, in certain embodiments, are isolated polypeptides or polypeptide complexes according to Formula I:

A₂-A₁-L₁-P₁-H₁ (Formula I)

wherein: A₁ comprises a first antigen recognizing molecule that binds to an effector cell antigen; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to TROP2. In some embodiments, the first antigen recognizing molecule comprises an antibody or antibody fragment. In some embodiments, first antigen recognizing molecule comprises an antibody or antibody fragment that is human or humanized. In some embodiments, L₁ is bound to N-terminus of the first antigen recognizing molecule. In some embodiments, A₂ is bound to C-terminus of the first antigen recognizing molecule. In some embodiments, L₁ is bound to C-terminus of the first antigen recognizing molecule. In some embodiments, A₂ is bound to N-terminus of the first antigen recognizing molecule. In some embodiments, the antibody or antibody fragment comprises a single chain variable fragment, a single domain antibody, or a Fab fragment. In some embodiments, A₁ is the single chain variable fragment (scFv). In some embodiments, the scFv comprises a scFv heavy chain polypeptide and a scFv light chain polypeptide. In some embodiments, A₁ is the single domain antibody. In some embodiments, the antibody or antibody fragment comprises a single chain variable fragment (scFv), a heavy chain variable domain (VH domain), a light chain variable domain (VL domain), or a variable domain (VHH) of a camelid derived single domain antibody. In some embodiments, A₁ comprises an anti-CD3e single chain variable fragment. In some embodiments, A₁ comprises an anti-CD3e single chain variable fragment that has a K_{D} binding of 1 μM or less to CD3 on CD3 expressing cells. In some embodiments, the effector cell antigen comprises CD3. In some embodiments, A₁ comprises a variable light chain and variable heavy chain each of which is capable of specifically binding to human CD3. In some embodiments, A₁ comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, 15865, 15865v12, 15865v16, and 15865v19. In some embodiments, the isolated polypeptide or polypeptide complex of Formula I binds to an effector cell when L₁ is cleaved by the tumor specific protease. In some embodiments, the isolated polypeptide or polypeptide complex of Formula I binds to an effector cell when L₁ is cleaved by the tumor specific protease and A₁ binds to the effector cell. In some embodiments, the effector cell is a T cell. In some embodiments, A₁ binds to a polypeptide that is part of a TCR-CD3 complex on the effector cell. In some embodiments, the polypeptide that is part of the TCR-CD3 complex is human CD3ε. In some embodiments, the effector cell antigen comprises CD3, wherein the scFv comprises complementarity determining regions (CDRs): HC-CDR1, HC-CDR2, and HC-CDR3, wherein the HC-CDR1, the HC-CDR2, and the HC-CDR3 of the scFv: HC-CDR1: SEQ ID NO: 1, HC-CDR2: SEQ ID NO: 2, and HC-CDR3: SEQ ID NO: 3; and the scFv comprises CDRs: LC-CDR1, LC-CDR2, and LC-CDR3, wherein the LC-CDR1, the LC-CDR2, and the LC-CDR3 of the scFv LC-CDR1: SEQ ID NO: 4, LC-CDR2: SEQ ID NO:5, and LC-CDR3: SEQ ID NO: 6. In some embodiments, the effector cell antigen comprises CD3, and the scFv comprises an amino acid sequence according to SEQ ID NO: 13. In some embodiments, the effector cell antigen comprises CD3, wherein the scFv comprises complementarity determining regions (CDRs): HC-CDR1, HC-CDR2, and HC-CDR3, wherein the HC-CDR1, the HC-CDR2, and the HC-CDR3 of the scFv: HC-CDR1: SEQ ID NO: 7, HC-CDR2: SEQ ID NO: 8, and HC-CDR3: SEQ ID NO: 9; and the scFv comprises CDRs: LC-CDR1, LC-CDR2, and LC-CDR3, wherein the LC-CDR1, the LC-CDR2, and the LC-CDR3 of the scFv LC-CDR1: SEQ ID NO: 10, LC-CDR2: SEQ ID NO: 11, and LC-CDR3: SEQ ID NO: 12. In some embodiments, the effector cell antigen comprises CD3, and the scFv comprises an amino acid sequence according to SEQ ID NO: 14. In some embodiments, second antigen recognizing molecule comprises an antibody or antibody fragment. In some embodiments, the antibody or antibody fragment thereof comprises a single chain variable fragment, a single domain antibody, or a Fab. In some embodiments, the antibody or antibody fragment thereof comprises a single chain variable fragment (scFv), a heavy chain variable domain (VH domain), a light chain variable domain (VL domain), a variable domain (VHH) of a camelid derived single domain antibody. In some embodiments, the antibody or antibody fragment thereof is humanized or human. In some embodiments, A₂ is the Fab. In some embodiments, the Fab comprises (a) a Fab light chain polypeptide and (b) a Fab heavy chain polypeptide. In some embodiments, the Fab comprises complementarity determining regions (CDRs): HC-CDR1, HC-CDR2, and HC-CDR3, wherein the HC-CDR1, the HC-CDR2, and the HC-CDR3 of the Fab comprise: HC-CDR1: SEQ ID NO: 15, HC-CDR2: SEQ ID NO: 16, and HC-CDR3: SEQ ID NO: 17; and the Fab comprises CDRs: LC-CDR1, LC-CDR2, and LC-CDR3, wherein the LC-CDR1, the LC-CDR2, and the LC-CDR3 of the Fab comprise LC-CDR1: SEQ ID NO: 18, LC-CDR2: SEQ ID NO: 19, and LC-CDR3: SEQ ID NO: 20. In some embodiments, the Fab light chain polypeptide comprises an amino acid sequence according to SEQ ID NO: 21. In some embodiments, Fab heavy chain polypeptide comprises an amino acid sequence according to SEQ ID NO: 22. In some embodiments, the Fab light chain polypeptide of A₂ is bound to a C-terminus of the single chain variable fragment (scFv) of A₁. In some embodiments, the Fab heavy chain polypeptide of A₂ is bound to a C-terminus of the single chain variable fragment (scFv) A₁. In some embodiments, the Fab light chain polypeptide of A₂ is bound to a N-terminus of the single chain variable fragment (scFv) of A₁. In some embodiments, the Fab heavy chain polypeptide of A₂ is bound to a N-terminus of the single chain variable fragment (scFv) A₁. In some embodiments, the Fab heavy chain polypeptide of A₂ is bound to the scFv heavy chain polypeptide of A₁. In some embodiments, the Fab light chain polypeptide of A₂ is bound to the scFv heavy chain polypeptide of A₁. In some embodiments, the Fab heavy chain polypeptide of A₂ is bound to the scFv light chain polypeptide of A₁. In some embodiments, the Fab light chain polypeptide of A₂ is bound to the scFv light chain polypeptide of A₁. In some embodiments, A₂ further comprises P₂ and L₂, wherein P₂ comprises a peptide that binds to A₂; and L₂ comprises a linking moiety that connects A₂ to P₂ and is a substrate for a tumor specific protease. In some embodiments, the isolated polypeptide or polypeptide complex is according to Formula Ia P₂-L₂-A₂-A₁-L₁-P₁-H₁ (Formula Ia) In some embodiments, the Fab heavy chain polypeptide of A₂ is bound to the scFv heavy chain polypeptide of A₁ and L₂ is bound to the Fab light chain polypeptide of A₂. In some embodiments, the Fab light chain polypeptide of A₂ is bound to the scFv heavy chain polypeptide of A₁ and L₂ is bound to the Fab heavy chain polypeptide of A₂. In some embodiments, the Fab heavy chain polypeptide of A₂ is bound to the scFv light chain polypeptide of A₁ and L₂ is bound to the Fab light chain polypeptide of A₂. In some embodiments, the Fab light chain polypeptide of A₂ is bound to the scFv light chain polypeptide of A₁ and L₂ is bound to the Fab heavy chain polypeptide of A₂. In some embodiments, P₁ impairs binding of A₁ to the effector cell antigen. In some embodiments, P₁ is bound to A₁ through ionic interactions, electrostatic interactions, hydrophobic interactions, Pi-stacking interactions, and H-bonding interactions, or a combination thereof. In some embodiments, P₁ has less than 70% sequence homology to the effector cell antigen. In some embodiments, P₂ impairs binding of A₂ to TROP2. In some embodiments, P₂ is bound to A₂ through ionic interactions, electrostatic interactions, hydrophobic interactions, Pi-stacking interactions, and H-bonding interactions, or a combination thereof. In some embodiments, P₂ is bound to A₂ at or near an antigen binding site. In some embodiments, P₂ has less than 70% sequence homology to TROP2. In some embodiments, P₁ or P₂ comprises a peptide sequence of at least 10 amino acids in length. In some embodiments, P₁ or P₂ comprises a peptide sequence of at least 10 amino acids in length and no more than 20 amino acids in length. In some embodiments, P₁ or P₂ comprises a peptide sequence of at least 16 amino acids in length. In some embodiments, P₁ or P₂ comprises a peptide sequence of no more than 40 amino acids in length. In some embodiments, P₁ or P₂ comprises at least two cysteine amino acid residues. In some embodiments, P₁ or P₂ comprises a cyclic peptide or a linear peptide. In some embodiments, P₁ or P₂ comprises a cyclic peptide. In some embodiments, P₁ or P₂ comprises a linear peptide. In some embodiments, P₁ comprises at least two cysteine amino acid residues. In some embodiments, P₁ comprises an amino acid sequence according to SEQ ID NO: 26, 27, or 122. In some embodiments, P₂ comprises an amino acid sequence according to any one of SEQ ID NOs: 23-25. In some embodiments, L₁ is bound to N-terminus of A₁. In some embodiments, L₁ is bound to C-terminus of A₁. In some embodiments, L₂ is bound to N-terminus of A₂. In some embodiments, L₂ is bound to C-terminus of A₂. In some embodiments, L₁ or L₂ is a peptide sequence having at least 5 to no more than 50 amino acids. In some embodiments, L₁ or L₂ is a peptide sequence having at least 10 to no more than 30 amino acids. In some embodiments, L₁ or L₂ is a peptide sequence having at least 10 amino acids. In some embodiments, L₁ or L₂ is a peptide sequence having at least 18 amino acids. In some embodiments, L₁ or L₂ is a peptide sequence having at least 26 amino acids. In some embodiments, L₁ or L₂ has a formula comprising (G₂S)ₙ (SEQ ID NO: 243), wherein n is an integer from 1 to 3. In some embodiments, L₁ has a formula selected from the group consisting of (G₂S)ₙ, (GS)ₙ, (GSGGS)ₙ (SEQ ID NO: 62), (GGGS)ₙ (SEQ ID NO: 63), (GGGGS)ₙ (SEQ ID NO: 64), and (GSSGGS)ₙ (SEQ ID NO: 65), wherein n is an integer of at least 1. In some embodiments, P₁ becomes unbound from A₁ when L₁ is cleaved by the tumor specific protease thereby exposing A₁ to the effector cell antigen. In some embodiments, P₂ becomes unbound from A₂ when L₂ is cleaved by the tumor specific protease thereby exposing A₂ to TROP2. In some embodiments, the tumor specific protease is selected from the group consisting of a matrix metalloprotease (MMP), serine protease, cysteine protease, threonine protease, and aspartic protease. In some embodiments, the matrix metalloprotease comprises MMP2, MMP7, MMP9, MMP13, or MMP14. In some embodiments, the serine protease comprises matriptase (MTSP1), urokinase, or hepsin. In some embodiments, L₁ or L₂ comprises a urokinase cleavable amino acid sequence, a matriptase cleavable amino acid sequence, matrix metalloprotease cleavable amino acid sequence, or a legumain cleavable amino acid sequence. In some embodiments, L₁ or L₂ comprises an amino acid sequence according to SEQ ID NO: 31 or 32. In some embodiments, L₁ or L₂ comprises an amino acid sequence according to SEQ ID NO: 58 or 59. In some embodiments, L₁ or L₂ comprises an amino acid sequence according to any one of SEQ ID NOs: 28-61. In some embodiments, L₁ or L₂ comprises an amino acid sequence of Linker 25 (ISSGLLSGRSDAG) (SEQ ID NO: 54), Linker 26 (AAGLLAPPGGLSGRSDAG) (SEQ ID NO: 55), Linker 27 (SPLGLSGRSDAG) (SEQ ID NO: 56), or Linker 28 (LSGRSDAGSPLGLAG) (SEQ ID NO: 57), or an amino acid sequence that has 1, 2, or 3 amino acid substitutions, additions, or deletions relative to the amino acid sequence of Linker 25, Linker 26, Linker 27, or Linker 28. In some embodiments, H₁ comprises a polymer. In some embodiments, the polymer is polyethylene glycol (PEG). In some embodiments, H₁ comprises albumin. In some embodiments, H₁ comprises an Fc domain. In some embodiments, the albumin is serum albumin. In some embodiments, the albumin is human serum albumin. In some embodiments, H₁ comprises a polypeptide, a ligand, or a small molecule. In some embodiments, the polypeptide, the ligand or the small molecule binds serum protein or a fragment thereof, a circulating immunoglobulin or a fragment thereof, or CD35/CR1. In some embodiments, the serum protein comprises a thyroxine-binding protein, a transthyretin, a 1-acid glycoprotein, a transferrin, transferrin receptor or a transferrin-binding portion thereof, a fibrinogen, or an albumin. In some embodiments, the circulating immunoglobulin molecule comprises IgG1, IgG2, IgG3, IgG4, slgA, IgM or IgD. In some embodiments, the serum protein is albumin. In some embodiments, the polypeptide is an antibody. In some embodiments, the antibody comprises a single domain antibody, a single chain variable fragment, or a Fab. In some embodiments, the single domain antibody comprises a single domain antibody that binds to albumin. In some embodiments, the single domain antibody is a human or humanized antibody. In some embodiments, the single domain antibody is 645gH1gL1. In some embodiments, the single domain antibody is 645dsgH5gL4. In some embodiments, the single domain antibody is 23-13-A01-sc02. In some embodiments, the single domain antibody is A10m3 or a fragment thereof. In some embodiments, the single domain antibody is DOM7r-31. In some embodiments, the single domain antibody is DOM7h-11-15. In some embodiments, the single domain antibody is Alb-1, Alb-8, or Alb-23. In some embodiments, the single domain antibody is 10E. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 66, HC-CDR2: SEQ ID NO: 67, and HC-CDR3: SEQ ID NO: 68. In some embodiments, the single domain antibody comprises an amino acid sequence according to SEQ ID NO: 69. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 70, HC-CDR2: SEQ ID NO: 71, and HC-CDR3: SEQ ID NO: 72. In some embodiments, the single domain antibody comprises an amino acid sequence according to SEQ ID NO: 73. In some embodiments, the single domain antibody is SA21. In some embodiments, the isolated polypeptide or polypeptide complex comprises a modified amino acid, a non-natural amino acid, a modified non-natural amino acid, or a combination thereof. In some embodiments, the modified amino acid or modified non-natural amino acid comprises a post-translational modification. In some embodiments, H₁ comprises a linking moiety (L₃) that connects H₁ to P₁. In some embodiments, L₃ is a peptide sequence having at least 5 to no more than 50 amino acids. In some embodiments, L₃ is a peptide sequence having at least 10 to no more than 30 amino acids. In some embodiments, L₃ is a peptide sequence having at least 10 amino acids. In some embodiments, L₃ is a peptide sequence having at least 18 amino acids. In some embodiments, L₃ is a peptide sequence having at least 26 amino acids. In some embodiments, L₃ has a formula selected from the group consisting of (G₂S)ₙ, (GS)ₙ, (GSGGS)ₙ (SEQ ID NO: 62), (GGGS)ₙ (SEQ ID NO: 63), (GGGGS)ₙ (SEQ ID NO: 64), and (GSSGGS)ₙ (SEQ ID NO: 65), wherein n is an integer of at least 1. In some embodiments, L₃ comprises an amino acid sequence according to SEQ ID NO: 30. In some embodiments, the isolated polypeptide or polypeptide complex comprises an amino acid sequence with at least 95% sequence identity to SEQ ID NOs: 74-121. In some embodiments, the isolated polypeptide or polypeptide complex comprises an amino acid sequence with at least 95% sequence identity to SEQ ID NO: 82. In some embodiments, the isolated polypeptide or polypeptide complex comprises an amino acid sequence with at least 95% sequence identity to SEQ ID NO: 83. In some embodiments, the isolated polypeptide or polypeptide complex comprises an amino acid sequence with at least 95% sequence identity to SEQ ID NO: 74 and SEQ ID NO: 75. In some embodiments, the isolated polypeptide or polypeptide complex comprises an amino acid sequence with at least 95% sequence identity to SEQ ID NO: 76 and SEQ ID NO: 77. In some embodiments, the isolated polypeptide or polypeptide complex comprises an amino acid sequence with at least 95% sequence identity to SEQ ID NO: 78 and SEQ ID NO: 79. In some embodiments, the isolated polypeptide or polypeptide complex comprises an amino acid sequence with at least 95% sequence identity to SEQ ID NO: 80 and SEQ ID NO: 81. In some embodiments, the isolated polypeptide or polypeptide complex comprises an amino acid sequence with at least 95% sequence identity to SEQ ID NO: 82 and SEQ ID NO: 83. In some embodiments, the isolated polypeptide or polypeptide complex comprises an amino acid sequence with at least 95% sequence identity to SEQ ID NO: 84 and SEQ ID NO: 85. In some embodiments, the isolated polypeptide or polypeptide complex comprises an amino acid sequence with at least 95% sequence identity to SEQ ID NO: 86 and SEQ ID NO: 87. In some embodiments, the isolated polypeptide or polypeptide complex comprises an amino acid sequence with at least 95% sequence identity to SEQ ID NO: 88 and SEQ ID NO: 89. In some embodiments, the isolated polypeptide or polypeptide complex comprises an amino acid sequence with at least 95% sequence identity to SEQ ID NO: 90 and SEQ ID NO: 91. In some embodiments, the isolated polypeptide or polypeptide complex comprises an amino acid sequence with at least 95% sequence identity to SEQ ID NO: 92 and SEQ ID NO: 93. In some embodiments, the isolated polypeptide or polypeptide complex comprises an amino acid sequence with at least 95% sequence identity to SEQ ID NO: 94 and SEQ ID NO: 95. In some embodiments, the isolated polypeptide or polypeptide complex comprises an amino acid sequence with at least 95% sequence identity to SEQ ID NO: 96 and SEQ ID NO: 97. In some embodiments, the isolated polypeptide or polypeptide complex comprises an amino acid sequence with at least 95% sequence identity to SEQ ID NO: 98 and SEQ ID NO: 99. In some embodiments, the isolated polypeptide or polypeptide complex comprises an amino acid sequence with at least 95% sequence identity to SEQ ID NO: 100 and SEQ ID NO: 101. In some embodiments, the isolated polypeptide or polypeptide complex comprises an amino acid sequence with at least 95% sequence identity to SEQ ID NO: 102 and SEQ ID NO: 103. In some embodiments, the isolated polypeptide or polypeptide complex comprises an amino acid sequence with at least 95% sequence identity to SEQ ID NO: 104 and SEQ ID NO: 105. In some embodiments, the isolated polypeptide or polypeptide complex comprises an amino acid sequence with at least 95% sequence identity to SEQ ID NO: 106 and SEQ ID NO: 107. In some embodiments, the isolated polypeptide or polypeptide complex comprises an amino acid sequence with at least 95% sequence identity to SEQ ID NO: 108 and SEQ ID NO: 109. In some embodiments, the isolated polypeptide or polypeptide complex comprises an amino acid sequence with at least 95% sequence identity to SEQ ID NO: 110 and SEQ ID NO: 111. In some embodiments, the isolated polypeptide or polypeptide complex comprises an amino acid sequence with at least 95% sequence identity to SEQ ID NO: 112 and SEQ ID NO: 113. In some embodiments, the isolated polypeptide or polypeptide complex comprises an amino acid sequence with at least 95% sequence identity to SEQ ID NO: 114 and SEQ ID NO: 115. In some embodiments, the isolated polypeptide or polypeptide complex comprises an amino acid sequence with at least 95% sequence identity to SEQ ID NO: 116 and SEQ ID NO: 117. In some embodiments, the isolated polypeptide or polypeptide complex comprises an amino acid sequence with at least 95% sequence identity to SEQ ID NO: 118 and SEQ ID NO: 119. In some embodiments, the isolated polypeptide or polypeptide complex comprises an amino acid sequence with at least 95% sequence identity to SEQ ID NO: 120 and SEQ ID NO: 121.

Disclosed herein, in certain embodiments, are pharmaceutical compositions comprising: (a) the isolated polypeptide or polypeptide complex described herein; and (b) a pharmaceutically acceptable excipient.

Disclosed herein, in certain embodiments, are isolated recombinant nucleic acid molecules encoding the isolated polypeptide or polypeptide complex described herein.

Disclosed herein, in certain embodiments, are isolated polypeptides or polypeptide complexes according to Formula II:

L₁ₐ-P₁ₐ-H₁ₐ (Formula II)

wherein: L₁ₐ comprises a tumor specific protease-cleaved linking moiety that when uncleaved connects P₁ₐ to a first antigen recognizing molecule that binds to an effector cell antigen and the first antigen recognizing molecule is connected to a second antigen recognizing molecule that binds to TROP2; P₁ₐ comprises a peptide that binds to the first antigen recognizing molecule when L₁ₐ is uncleaved; and H₁ₐ comprises a half-life extending molecule. In some embodiments, P₁ₐ when L₁ₐ is uncleaved impairs binding of the first antigen recognizing molecule to the effector cell antigen. In some embodiments, the first antigen recognizing molecule comprises an antibody or antibody fragment. In some embodiments, the effector cell antigen is an anti-CD3 effector cell antigen. In some embodiments, P₁ₐ has less than 70% sequence homology to the effector cell antigen. In some embodiments, P₁ₐ comprises a peptide sequence of at least 10 amino acids in length. In some embodiments, P₁ₐ comprises a peptide sequence of at least 10 amino acids in length and no more than 20 amino acids in length. In some embodiments, P₁ₐ comprises a peptide sequence of at least 16 amino acids in length. In some embodiments, P₁ₐ comprises a peptide sequence of no more than 40 amino acids in length. In some embodiments, P₁ₐ comprises at least two cysteine amino acid residues. In some embodiments, P₁ₐ comprises a cyclic peptide or a linear peptide. In some embodiments, P₁ₐ comprises a cyclic peptide. In some embodiments, P₁ₐ comprises a linear peptide. In some embodiments, P₁ₐ comprises an amino acid sequence selected from the group consisting of SEQ ID NO: 26, 27, or 122. In some embodiments, H₁ₐ comprises a polymer. In some embodiments, the polymer is polyethylene glycol (PEG). In some embodiments, H₁ₐ comprises albumin. In some embodiments, H₁ₐ comprises an Fc domain. In some embodiments, the albumin is serum albumin. In some embodiments, the albumin is human serum albumin. In some embodiments, H₁ₐ comprises a polypeptide, a ligand, or a small molecule. In some embodiments, the polypeptide, the ligand or the small molecule binds a serum protein or a fragment thereof, a circulating immunoglobulin or a fragment thereof, or CD35/CR1. In some embodiments, the serum protein comprises a thyroxine-binding protein, a transthyretin, a 1-acid glycoprotein, a transferrin, transferrin receptor or a transferrin-binding portion thereof, a fibrinogen, or an albumin. In some embodiments, the circulating immunoglobulin molecule comprises IgG1, IgG2, IgG3, IgG4, slgA, IgM or IgD. In some embodiments, the serum protein is albumin. In some embodiments, the polypeptide is an antibody. In some embodiments, the antibody comprises a single domain antibody, a single chain variable fragment or a Fab. In some embodiments, the antibody comprises a single domain antibody that binds to albumin. In some embodiments, the antibody is a human or humanized antibody. In some embodiments, the single domain antibody is 645gH1gL1. In some embodiments, the single domain antibody is 645dsgH5gL4. In some embodiments, the single domain antibody is 23-13-A01-sc02. In some embodiments, the single domain antibody is A10m3 or a fragment thereof. In some embodiments, the single domain antibody is DOM7r-31. In some embodiments, the single domain antibody is DOM7h-11-15. In some embodiments, the single domain antibody is Alb-1, Alb-8, or Alb-23. In some embodiments, the single domain antibody is 10E. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 66, HC-CDR2: SEQ ID NO: 67, and HC-CDR3: SEQ ID NO: 68. In some embodiments, the single domain antibody comprises an amino acid sequence according to SEQ ID NO: 69. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 70, HC-CDR2: SEQ ID NO: 71, and HC-CDR3: SEQ ID NO: 72. In some embodiments, the single domain antibody comprises an amino acid sequence according to SEQ ID NO: 73. In some embodiments, the single domain antibody is SA21. In some embodiments, H₁ₐ comprises a linking moiety (L₁ₐ) that connects H₁ₐ to Pia. In some embodiments, L₁ₐ is a peptide sequence having at least 5 to no more than 50 amino acids. In some embodiments, L₁ₐ is a peptide sequence having at least 10 to no more than 30 amino acids. In some embodiments, L₁ₐ is a peptide sequence having at least 10 amino acids. In some embodiments, L₁ₐ is a peptide sequence having at least 18 amino acids. In some embodiments, L₁ₐ is a peptide sequence having at least 26 amino acids. In some embodiments, L₁ₐ has a formula selected from the group consisting of (G₂S)ₙ, (GS)ₙ, (GSGGS)ₙ (SEQ ID NO: 62), (GGGS)ₙ (SEQ ID NO: 63), (GGGGS)ₙ (SEQ ID NO: 64), and (GSSGGS)ₙ (SEQ ID NO: 65), wherein n is an integer of at least 1. In some embodiments, L₁ₐ comprises an amino acid sequence according to any one of SEQ ID NOs: 28-61. Disclosed herein, in some embodiments, are polypeptide complexes comprising a structural arrangement according to Configuration 1:
wherein the polypeptide complex comprises a single chain variable fragment (scFv) comprising a light chain variable domain and a heavy chain variable domain, wherein the scFv further comprises a peptide that impairs binding of the scFv to an effector cell antigen and the peptide is linked to a N-terminus of the heavy chain variable domain of the scFv with a linking moiety that is a substrate for a tumor specific protease, and the peptide further comprises a half-life extending molecule; and a Fab that binds to TROP2, wherein the Fab comprises a Fab light chain polypeptide and a Fab heavy chain polypeptide, wherein the Fab heavy chain polypeptide is linked to a C terminus of the light chain variable domain of the scFv, and wherein the Fab further comprises P₂ and L₂, wherein P₂ comprises a peptide that impairs binding to TROP2; and L₂ comprises a linking moiety that connects the Fab light chain polypeptide to P₂ and is a substrate for a tumor specific protease. Disclosed herein, in some embodiments, are polypeptide complexes comprising a structural arrangement according to Configuration 2:
wherein the polypeptide complex comprises a single chain variable fragment (scFv) comprising a light chain variable domain and a heavy chain variable domain, wherein the scFv further comprises a peptide that impairs binding of the scFv to an effector cell antigen and the peptide is linked to a N-terminus of the heavy chain variable domain of the scFv with a linking moiety that is a substrate for a tumor specific protease, and the peptide further comprises a half-life extending molecule; and a Fab that binds to TROP2, wherein the Fab comprises a Fab light chain polypeptide and a Fab heavy chain polypeptide, wherein the Fab light chain polypeptide is linked to a C terminus of the light chain variable domain of the scFv, and wherein the Fab further comprises P₂ and L₂, wherein P₂ comprises a peptide that impairs binding to TROP2; and L₂ comprises a linking moiety that connects the Fab heavy chain polypeptide to P₂ and is a substrate for a tumor specific protease.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1B illustrates polypeptide complexes of this disclosure in a normal orientation (FIG. 1A) and flipped orientation (FIG. 1B).

FIG. 2A illustrates titration data for TROP2 binding for several polypeptide complexes of this disclosure.

FIG. 2B illustrates titration data for TROP2 binding for several polypeptide complexes of this disclosure.

FIG. 3A illustrates titration data for CD3ε binding for several polypeptide complexes of this disclosure.

FIG. 3B illustrates titration data for CD3ε binding for several polypeptide complexes of this disclosure.

FIG. 4A illustrates cell viability data for HCT116 cells treated with polypeptide complexes of this disclosure.

FIG. 4B illustrates cell viability data for HCT116 cells treated with polypeptide complexes of this disclosure.

FIG. 4C illustrates cell viability data for MDAMB231 cells treated with polypeptide complexes of this disclosure.

FIG. 4D illustrates cell viability data for MDAMB231 cells treated with polypeptide complexes of this disclosure.

FIG. 5A illustrates titration data for TROP2 binding for several polypeptide complexes of this disclosure.

FIG. 5B illustrates titration data for TROP2 binding for several polypeptide complexes of this disclosure.

FIG. 6A illustrates titration data for CD3ε binding for several polypeptide complexes of this disclosure.

FIG. 6B illustrates titration data for CD3ε binding for several polypeptide complexes of this disclosure.

FIG. 7A illustrates cell viability data for HCT116 cells treated with polypeptide complexes of this disclosure.

FIG. 7B illustrates cell viability data for HCT116 cells treated with polypeptide complexes of this disclosure.

FIGS. 8A-8C illustrate polypeptide complex mediated H292 tumor cell killing in the presence of CD8+ T cells.

FIGS. 9A-9D illustrate polypeptide complex mediated HCT116 tumor cell killing in the presence of CD8+ T cells.

FIGS. 10A-10C illustrate polypeptide complex mediated MDAMB231 tumor cell killing in the presence of CD8+ T cells.

FIG. 11A illustrates polypeptide complex PC1 pharmacokinetics in cynomolgus monkeys after a single IV bolus injection.

FIG. 11B illustrates polypeptide complex PC5 pharmacokinetics in cynomolgus monkeys after a single IV bolus injection.

FIG. 11C illustrates polypeptide complex PC18 pharmacokinetics in cynomolgus monkeys after a single IV bolus injection.

FIG. 11D illustrates polypeptide complex PC21 pharmacokinetics in cynomolgus monkeys after a single IV bolus injection.

FIG. 12A illustrates cytokine release in cynomolgus monkeys after single IV bolus of PC1.

FIG. 12B illustrates cytokine release in cynomolgus monkeys after single IV bolus of polypeptide complex PC5.

FIG. 12C illustrates cytokine release in cynomolgus monkeys after single IV bolus of polypeptide complex PC18.

FIG. 12D illustrates cytokine release in cynomolgus monkeys after single IV bolus of polypeptide complex PC21.

FIG. 12E illustrates plasma cytokine level after administration of polypeptide complexes (unmasked TROP2-TCE and masked TROP2-TRACTr) described herein.

FIG. 13A illustrates serum liver enzymes in cynomolgus monkeys after single IV bolus of PC1.

FIG. 13B illustrates serum liver enzymes in cynomolgus monkeys after single IV bolus of polypeptide complex PC5.

FIG. 13C illustrates serum liver enzymes in cynomolgus monkeys after single IV bolus of polypeptide complex PC18.

FIG. 13D illustrates serum liver enzymes in cynomolgus monkeys after single IV bolus of polypeptide complex PC21.

FIG. 14A and FIG. 14B illustrate TROP2 Fab inhibition by alanine scanning peptides of TROP2 Fab Peptide-1 as measured by ELISA.

FIG. 15A and FIG. 15B illustrate TROP2 Fab binding by alanine scanning peptides of TROP2 Fab Peptide-2 as measured by ELISA.

FIG. 16A and FIG. 16B illustrate TROP2 Fab inhibition by alanine scanning peptides of TROP2 Fab Peptide-2 as measured by ELISA.

FIG. 17A-FIG. 17C illustrate optimized TROP2 Fab Peptide-1 sequences evaluated for peptide inhibition of TROP2 Fab.

FIG. 18 illustrates the core sequence motif of optimized TROP2 Fab Peptide-1 sequences generated using WebLogo 3.7.4.

FIG. 19A-FIG. 19C illustrate optimized TROP2 Fab Peptide-2 sequences evaluated for peptide binding to TROP2 Fab.

FIG. 20A-FIG. 20C illustrate optimized TROP2 Fab Peptide-2 sequences evaluated for peptide inhibition of TROP2 Fab.

FIG. 21 illustrates the core sequence motif of optimized TROP2 Fab Peptide-2 sequences generated using WebLogo 3.7.4.

FIG. 22 illustrates titration data for TROP2 binding for several polypeptide complexes of this disclosure.

FIG. 23 illustrates titration data for CD3ε binding for several polypeptide complexes of this disclosure.

FIG. 24A illustrates PC25 mediated HCT116 tumor cell killing in the presence of CD8+ T cells.

FIG. 24B illustrates PC26 mediated HCT116 tumor cell killing in the presence of CD8+ T cells.

FIG. 24C illustrates PC25 mediated MDAMB231 tumor cell killing in the presence of CD8+ T cells.

FIG. 25 illustrates PC22 pharmacokinetics in cynomolgus monkeys after a single IV bolus injection.

FIGS. 26A-26F illustrate cytokine release in cynomolgus monkeys after single IV bolus of PC22.

FIGS. 27A-27B illustrate serum liver enzyme levels in cynomolgus monkeys after single IV bolus of PC22.

FIGS. 28A-28D illustrate in vivo tumor growth inhibition in human PBMC engrafted NCG mice bearing MDAMB231 xenograft tumors. The anti-tumor activity observed was protease dependent in that the polypeptide complex lacking the protease substrate within the cleavable linker was equivalent to vehicle controls. Shown are PC3 (FIG. 28A), PC17 (FIG. 28B), PC23 (FIG. 28C), PC24 (FIG. 28D).

FIGS. 29A-29F illustrate anti-CD3 scFv binding by alanine scanning peptides of anti-CD3 scFv Peptide-A and Peptide-B as measured by ELISA.

FIGS. 30A-30F illustrate inhibition of anti-CD3 scFv binding to CD3 by alanine scanning peptides of anti-CD3 scFv Peptide-A and Peptide-B as measured by ELISA.

FIGS. 31A-31B illustrate anti-CD3 scFv binding by optimized anti-CD3 scFv Peptide-B sequences as measured by ELISA.

FIGS. 32A-32B illustrate inhibition of anti-CD3 scFv binding to CD3 by optimized anti-CD3 scFv Peptide-B sequences as measured by ELISA.

FIG. 33 illustrates the core sequence motif of optimized anti-CD3 scFv Peptide-B sequences generated using WebLogo 3.7.4.

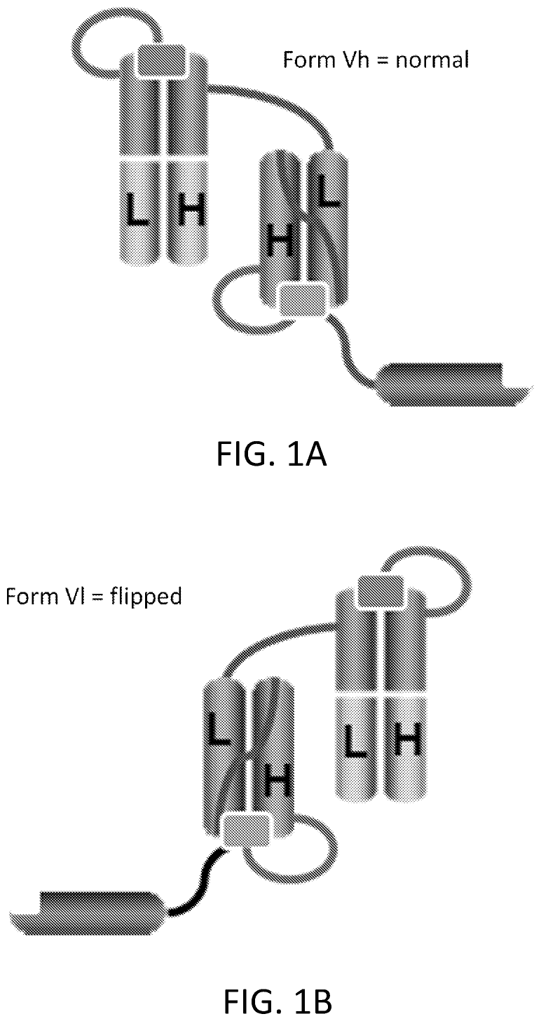

### DETAILED DESCRIPTION

Multispecific antibodies combine the benefits of different binding specificities derived from two or more antibodies into a single composition. Multispecific antibodies for redirecting T cells to cancers have shown promise in both pre-clinical and clinical studies. This approach relies on binding of one antigen interacting portion of the antibody to a tumor-associated antigen or marker, while a second antigen interacting portion can bind to an effector cell antigen on a T cell, such as CD3, which then triggers cytotoxic activity.

One such tumor-associated antigen is TROP2. TROP2 (also known as tumor-associated calcium signal transducer 2 or epithelial glycoprotein-1) is the protein product of the TACSTD2 gene, and is a transmembrane glycoprotein that functions in variety of cell signaling pathways, many of which are associated with tumorigenesis. TROP2 has been shown to be overexpressed in multiple human carcinomas, including lung, breast, cervical, ovarian, colorectal, pancreatic, and gastric cancers, and its expression has been correlated with poor patient prognosis. Furthermore, TROP2 functions as an oncogene capable of driving both tumorigenesis and metastasis in epithelial cancers such as colorectal cancer. TROP2 expression in cancer cells has long been correlated with drug resistance, and high levels of TROP2 expression have been shown to correlate with poor prognosis in a variety of cancer types. In a meta-analysis, including data from approximately 2,500 patients, increased TROP2 expression was associated with poor overall survival and disease-free survival outcomes across several solid tumors.

T cell engagers (TCEs) therapeutics have several benefits including they are not cell therapies and thus can be offered as off-the-shelf therapies as opposed to chimeric antigen receptor T cell (CAR T cell) therapies. While TCE therapeutics have displayed potent anti-tumor activity in hematological cancers, developing TCEs to treat solid tumors has faced challenges due to the limitations of prior TCE technologies, namely (i) overactivation of the immune system leading to cytokine release syndrome (CRS), (ii) on-target, healthy tissue toxicities and (iii) poor pharmacokinetics (PK) leading to short half-life. CRS arises from the systemic activation of T cells and can result in life-threatening elevations in inflammatory cytokines such as interleukin-6 (IL-6). Severe and acute CRS leading to dose limited toxicities and deaths have been observed upon the dosing of T cell engagers develop using other platforms to treat cancer patients in poor clinical studies. This toxicity restricts the maximum blood levels of T cell engagers that can be safely dosed. T cell engager effectiveness has also been limited because of on-target, healthy tissue toxicity. T cell engagers developed using a platform not designed for tumor-specification activation have resulted in clinicals holds and dose-limiting toxicities resulting from target expression in healthy tissues. T cell engagers have also been limited by short half-lives. T cell engagers quickly reach sub-therapeutic levels after being administered as they are quickly eliminated from the body due to their short exposure half-lives. For this reason, T cell engagers such as blinatumomab are typically administered by a low-dose, continuous infusion pump over a period of weeks to overcome the challenge of a short half-life and to maintain therapeutic levels of drug in the body. A continuous dosing regimen represents a significant burden for patients.

To overcome these challenges associated with the effectiveness of T cell engagers, described herein, are isolated polypeptide or polypeptide complexes that comprise binding domains that selectively bind to an effector cell antigen and TROP2, in which one or more of the binding domains is selectively activated in the tumor microenvironment and the isolated polypeptide or polypeptide complex comprises a half-life extending molecule. Such modifications reduce CRS and on-target healthy tissue toxicity risk, improves stability in the bloodstream and serum half-life prior to activation. The isolated polypeptide or polypeptide complexes described herein have activity at low levels of target expression, and are easily manufactured.

In some embodiments, the isolated polypeptides or polypeptide complexes described herein are used in a method of treating cancer. In some embodiments, the cancer has cells that express TROP2. In some instances, the cancer is a solid tumor cancer. In some embodiments, the cancer is lung, breast (e.g. HER2+; ER/PR+; TNBC), cervical, ovarian, colorectal, pancreatic or gastric. In some embodiments, the polypeptides or polypeptide complexes described herein are used in a method of treating triple-negative breast cancer (TNBC), urothelial cancer (UC), non-small cell lung cancer (NSCLC), small cell lung cancer (SCLC), gastric cancer, esophageal cancer, head and neck cancer, prostate cancer, or endometrial cancer. In some embodiments, the polypeptides or polypeptide complexes described herein are used in a method of treating breast cancer, lung cancer, urothelial cancer, endometrial cancer, ovarian cancer, prostate cancer, pancreatic cancer, gastric cancer, colon cancer, head and neck cancer, and glioma. In some embodiments, are methods of treating cancer comprising administering to a subject in need thereof an isolated polypeptide or polypeptide complex according to Formula I:

A₂-A₁-L₁-P₁-H₁ (Formula I)

wherein: A₁ comprises a first antigen recognizing molecule that binds to an effector cell antigen; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to TROP2.

Disclosed herein, in some embodiments, are isolated polypeptides or polypeptide complexes according to Formula I:

A₂-A₁-L₁-P₁-H₁ (Formula I)

wherein: A₁ comprises a first antigen recognizing molecule that binds to an effector cell antigen; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to TROP2.

Disclosed herein, in some embodiments, are isolated polypeptides or polypeptide complexes according to Formula I:

A₂-A₁-L₁-P₁-H₁ (Formula I)

wherein: A₁ is a first antigen recognizing molecule that binds to an effector cell antigen; P₁ is a peptide that binds to A₁; L₁ is a linking moiety that connects A₁ to P₁ and is a substrate for a tumor specific protease; H₁ is a half-life extending molecule; and A₂ is a second antigen recognizing molecule that binds to TROP2.

Disclosed herein, in some embodiments, are isolated polypeptides or polypeptide complexes comprising Formula I:

A₂-A₁-L₁-P₁-H₁ (Formula I)

wherein: A₁ comprises a first antigen recognizing molecule that binds to an effector cell antigen; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to TROP2.

Disclosed herein, in some embodiments, are isolated polypeptides or polypeptide complexes comprising Formula I:

A₂-A₁-L₁-P₁-H₁ (Formula I)

wherein: A₁ is a first antigen recognizing molecule that binds to an effector cell antigen; P₁ is a peptide that binds to A₁; L₁ is a linking moiety that connects A₁ to P₁ and is a substrate for a tumor specific protease; H₁ is a half-life extending molecule; and A₂ is a second antigen recognizing molecule that binds to TROP2.

Disclosed herein, in some embodiments, are isolated polypeptides or polypeptide complexes according to Formula I:

A₂-A₁-L₁-P₁-H₁ (Formula I)

wherein: A₁ comprises a first antigen recognizing molecule that binds to TROP2; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to an effector cell antigen.

Disclosed herein, in some embodiments, are isolated polypeptides or polypeptide complexes according to Formula I:

A₂-A₁-L₁-P₁-H₁ (Formula I)

wherein: A₁ is a first antigen recognizing molecule that binds to TROP2; P₁ is a peptide that binds to A₁; L₁ is a linking moiety that connects A₁ to P₁ and is a substrate for a tumor specific protease; H₁ is a half-life extending molecule; and A₂ is a second antigen recognizing molecule that binds to effector cell antigen.

Disclosed herein, in some embodiments, are isolated polypeptides or polypeptide complexes comprising Formula I:

A₂-A₁-L₁-P₁-H₁ (Formula I)

wherein: A₁ comprises a first antigen recognizing molecule that binds to TROP2; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to an effector cell antigen.

Disclosed herein, in some embodiments, are isolated polypeptides or polypeptide complexes comprising Formula I:

A₂-A₁-L₁-P₁-H₁ (Formula I)

wherein: A₁ is a first antigen recognizing molecule that binds to TROP2; P₁ is a peptide that binds to A₁; L₁ is a linking moiety that connects A₁ to P₁ and is a substrate for a tumor specific protease; H₁ is a half-life extending molecule; and A₂ is a second antigen recognizing molecule that binds to an effector cell antigen.

Disclosed herein, in some embodiments, are isolated polypeptides or polypeptide complexes according to Formula Ia:

P₂-L₂-A₂-A₁-L₁-P₁-H₁ (Formula Ia)

wherein A₂ further comprises P₂ and L₂, wherein P₂ comprises a peptide that binds to A₂; and L₂ comprises a linking moiety that connects A₂ to P₂ and is a substrate for a tumor specific protease.

Disclosed herein, in some embodiments, are isolated polypeptides or polypeptide complexes according to Formula Ia:

P₂-L₂-A₂-A₁-L₁-P₁-H₁ (Formula Ia)

wherein A₂ further comprises P₂ and L₂, wherein P₂ is a peptide that binds to A₂; and L₂ is a linking moiety that connects A₂ to P₂ and is a substrate for a tumor specific protease.

Disclosed herein, in some embodiments, are isolated polypeptides or polypeptide complexes comprising Formula Ia:

P₂-L₂-A₂-A₁-L₁-P₁-H₁ (Formula Ia)

wherein A₂ further comprises P₂ and L₂, wherein P₂ comprises a peptide that binds to A₂; and L₂ comprises a linking moiety that connects A₂ to P₂ and is a substrate for a tumor specific protease.

Disclosed herein, in some embodiments, are isolated polypeptides or polypeptide complexes comprising Formula Ia:

P₂-L₂-A₂-A₁-L₁-P₁-H₁ (Formula Ia)

wherein A₂ further comprises P₂ and L₂, wherein P₂ is a peptide that binds to A₂; and L₂ is a linking moiety that connects A₂ to P₂ and is a substrate for a tumor specific protease.

Disclosed herein, in some embodiments, are isolated polypeptides or polypeptide complexes according to Formula II:

L₁ₐ-P₁ₐ-H₁ₐ (Formula II)

wherein: L₁ₐ comprises a tumor specific protease-cleaved linking moiety that when uncleaved connects P₁ₐ to a first antigen recognizing molecule that binds to an effector cell antigen and the first antigen recognizing molecule is connected to a second antigen recognizing molecule that binds to TROP2; P₁ₐ comprises a peptide that binds to the first antigen recognizing molecule when L₁ₐ is uncleaved; and H₁ₐ comprises a half-life extending molecule.

Disclosed herein, in some embodiments, are isolated polypeptides or polypeptide complexes comprising Formula II:

L₁ₐ-P₁ₐ-H₁ₐ (Formula II)

wherein: L₁ₐ comprises a tumor specific protease-cleaved linking moiety that when uncleaved connects P₁ₐ to a first antigen recognizing molecule that binds to an effector cell antigen and the first antigen recognizing molecule is connected to a second antigen recognizing molecule that binds to TROP2; P₁ₐ comprises a peptide that binds to the first antigen recognizing molecule when L₁ₐ is uncleaved; and H₁ₐ comprises a half-life extending molecule.

Disclosed herein, in some embodiments, are isolated polypeptides or polypeptide complexes according to Formula II:

L₁ₐ-P₁ₐ-H₁ₐ (Formula II)

wherein: L₁ₐ is a tumor specific protease-cleaved linking moiety that when uncleaved connects P₁ₐ to a first antigen recognizing molecule that binds to an effector cell antigen and the first antigen recognizing molecule is connected to a second antigen recognizing molecule that binds to TROP2; P₁ₐ is a peptide that binds to the first antigen recognizing molecule when L₁ₐ is uncleaved; and H₁ₐ is a half-life extending molecule.

Disclosed herein, in some embodiments, are isolated polypeptides or polypeptide complexes comprising Formula II:

L₁ₐ-P₁ₐ-H₁ₐ (Formula II)

wherein: L₁ₐ is a tumor specific protease-cleaved linking moiety that when uncleaved connects P₁ₐ to a first antigen recognizing molecule that binds to an effector cell antigen and the first antigen recognizing molecule is connected to a second antigen recognizing molecule that binds to TROP2; P₁ₐ is a peptide that binds to the first antigen recognizing molecule when L₁ₐ is uncleaved; and H₁ₐ is a half-life extending molecule.

### First Antigen Recognizing Molecule (A₁)

Disclosed herein, in some embodiments, are polypeptides or polypeptide complexes, wherein the first antigen recognizing molecule binds to an effector cell antigen and the second antigen recognizing molecule binds to TROP2. In some embodiments, the effector cell antigen comprises CD3. In some embodiments, A₁ comprises a first antigen recognizing molecule that binds to an effector cell antigen.

In some embodiments, A₁ comprises an antibody or antibody fragment. In some embodiments, A₁ comprises an antibody or antibody fragment that is human or humanized. In some embodiments, L₁ is bound to N-terminus of the antibody or antibody fragment. In some embodiments, L₁ is bound to N-terminus of the antibody or antibody fragment and A₂ is bound to the other N-terminus of the antibody or antibody fragment. In some embodiments, A₂ is bound to C-terminus of the antibody or antibody fragment. In some embodiments, L₁ is bound to C-terminus of the antibody or antibody fragment. In some embodiments, A₂ is bound to N-terminus of the antibody or antibody fragment. In some embodiments, the antibody or antibody fragment comprises a single chain variable fragment, a single domain antibody, or a Fab fragment. In some embodiments, A₁ is the single chain variable fragment (scFv). In some embodiments, the scFv comprises a scFv heavy chain polypeptide and a scFv light chain polypeptide. In some embodiments, A₁ is the single domain antibody. In some embodiments, A₁ comprises a variable light chain and variable heavy chain each of which is capable of specifically binding to human CD3. In some embodiments, the effector cell antigen comprises CD3. In some embodiments, A₁ comprises an anti-CD3e single chain variable fragment. In some embodiments, A₁ comprises an anti-CD3e single chain variable fragment that has a K_{D} binding of 1 μM or less to CD3 on CD3 expressing cells. In some embodiments, A₁ comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, 15865, 15865v12, 15865v16, and 15865v19.

In some embodiments, A₁ comprises a first antigen recognizing molecule that binds TROP2. In some embodiments, A₁ comprises a variable light chain and variable heavy chain each of which is capable of specifically binding to human TROP2.

In some embodiments, the scFv that binds to CD3 comprises a scFv light chain variable domain and a scFv heavy chain variable domain. In some embodiments, the scFv heavy chain variable domain comprises at least one, two, or three complementarity determining regions (CDR)s disclosed in Table 1 or a sequence substantially identical thereto (e.g., a sequence that has at least 90%, 95%, 96%, 97%, 98%, or 99% sequence identity). In some embodiments, the scFv light chain variable domain comprises at least one, two, or three complementarity determining regions (CDR)s disclosed in Table 1 or a sequence substantially identical thereto (e.g., a sequence that has at least 90%, 95%, 96%, 97%, 98%, or 99% sequence identity).

In some embodiments, the scFv heavy chain variable domain comprises at least one, two, or three complementarity determining regions (CDR)s disclosed in Table 1 or a sequence substantially identical thereto (e.g., a sequence that has at least 90%, 95%, 96%, 97%, 98%, or 99% sequence identity); and the scFv light chain variable domain comprises at least one, two, or three complementarity determining regions (CDR)s disclosed in Table 1 or a sequence substantially identical thereto (e.g., a sequence that has at least 90%, 95%, 96%, 97%, 98%, or 99% sequence identity).

TABLE 1
anti-CD3 amino acid sequences (CDRs as determined by IMGT numbering system)

| | Amino Acid Sequence | SEQ ID |
|---|---|---|
| Construct Description | (N to C) | NO: |
| SP34.185 CD3: HC: CDR1 | GFTFNKYA | 1 |
| SP34.185 CD3: HC: CDR2 | IRSKYNNYAT | 2 |
| SP34.185 CD3: HC: CDR3 | VRHGNFGNSYISYWAY | 3 |
| SP34.185 CD3: LC: CDR1 | TGAVTSGNY | 4 |
| SP34.185 CD3: LC: CDR2 | GTK | 5 |
| SP34.185 CD3: LC: CDR3 | VLWYSNRWV | 6 |
| SP34.194 CD3: HC: CDR1 | GFTFNTYA | 7 |
| SP34.194 CD3: HC: CDR2 | IRSKYNNYAT | 8 |
| SP34.194 CD3: HC: CDR3 | VRHGNFGNSYVSWFAY | 9 |
| SP34.194 CD3: LC: CDR1 | TGAVTTSNY | 10 |
| SP34.194 CD3: LC: CDR2 | GT | 11 |
| SP34.194 CD3: LC: CDR3 | ALWYSNLWV | 12 |
| SP34.185 scFv | EVQLVESGGGLVQPGGSLKLSCA | 13 |
| (VH-linker 1-VL) | ASGFTFNKYAMNWVRQAPGKG | |
| | LEWVARIRSKYNNYATYYADSV | |
| | KDRFTISRDDSKNTAYLQMNNLK | |
| | TEDTAVYYCVRHGNFGNSYISY | |
| | WAYWGQGTLVTVSSGGGGSGGG | |
| | GSGGGGSQTVVTQEPSLTVSPGG | |
| | TVTLTCGSSTGAVTSGNYPNWV | |
| | QQKPGQAPRGLIGGTKFLAPGTP | |
| | ARFSGSLLGGKAALTLSGVQPED | |
| | EAEYYCVLWYSNRWVFGGGTKL | |
| | TVL | |
| SP34.194 scFv | QTVVTQEPSLTVSPGGTVTLTCRS | 14 |
| (VL-linker 1-VH) | STGAVTTSNYANWVQQKPGQAP | |
| | RGLIGGTNKRAPGTPARFSGSLLG | |
| | GKAALTLSGVQPEDEAEYYCAL | |
| | WYSNLWVFGGGTKLTVLGGGGS | |
| | GGGGSGGGGSEVQLVESGGGLV | |
| | QPGGSLKLSCAASGFTFNTYAMN | |
| | WVRQAPGKGLEWVARIRSKYNN | |
| | YATYYADSVKDRFTISRDDSKNT | |
| | AYLQMNNLKTEDTAVYYCVRHG | |
| | NFGNSYVSWFAYWGQGTLVTVS | |
| | S | |

In some embodiments, the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise: HC-CDR1: SEQ ID NO: 1; HC-CDR2: SEQ ID NO: 2; HC-CDR3: SEQ ID NO: 3, and wherein the CDRs comprise from 0-2 amino acid modifications in at least one of the HC-CDR1, HC-CDR2, or HC-CDR3. In some embodiments, the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise: HC-CDR1: SEQ ID NO: 7; HC-CDR2: SEQ ID NO: 8; HC-CDR3: SEQ ID NO: 9, and wherein the CDRs comprise from 0-2 amino acid modifications in at least one of the HC-CDR1, HC-CDR2, or HC-CDR3. In some embodiments, the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR11: SEQ ID NO: 4; LC-CDR2: SEQ ID NO: 5; and LC-CDR3: SEQ ID NO: 6, and wherein the CDRs comprise from 0-2 amino acid modifications in at least one of the LC-CDR1, LC-CDR2, or LC-CDR3. In some embodiments, the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR1: SEQ ID NO: 10; LC-CDR2: SEQ ID NO: 11; and LC-CDR3: SEQ ID NO: 12, and wherein the CDRs comprise from 0-2 amino acid modifications in at least one of the LC-CDR1, LC-CDR2, or LC-CDR3.

In some embodiments, the effector cell antigen comprises CD3, wherein A₁ comprises complementarity determining regions (CDRs): HC-CDR1, HC-CDR2, and HC-CDR3, wherein the HC-CDR1, the HC-CDR2, and the HC-CDR3 of A₁ comprise: HC-CDR1: SEQ ID NO: 1, HC-CDR2: SEQ ID NO: 2, and HC-CDR3: SEQ ID NO: 3; and A₁ comprises CDRs: LC-CDR1, LC-CDR2, and LC-CDR3, wherein the LC-CDR1, the LC-CDR2, and the LC-CDR3 of A₁ comprise LC-CDR1: SEQ ID NO: 4, LC-CDR2: SEQ ID NO:5, and LC-CDR3: SEQ ID NO: 6.

In some embodiments, the effector cell antigen comprises CD3, wherein A₁ comprises complementarity determining regions (CDRs): HC-CDR1, HC-CDR2, and HC-CDR3, wherein the HC-CDR1, the HC-CDR2, and the HC-CDR3 of A₁ comprise: HC-CDR1: SEQ ID NO: 7, HC-CDR2: SEQ ID NO: 8, and HC-CDR3: SEQ ID NO: 9; and A₁ comprises CDRs: LC-CDR1, LC-CDR2, and LC-CDR3, wherein the LC-CDR1, the LC-CDR2, and the LC-CDR3 of A₁ comprise LC-CDR1: SEQ ID NO: 10, LC-CDR2: SEQ ID NO: 11, and LC-CDR3: SEQ ID NO: 12.

In some embodiments, the isolated polypeptide or polypeptide complex of Formula I binds to an effector cell when L₁ is cleaved by the tumor specific protease. In some embodiments, the isolated polypeptide or polypeptide complex of Formula I binds to an effector cell when L₁ is cleaved by the tumor specific protease and A₁ binds to the effector cell. In some embodiments, the effector cell is a T cell. In some embodiments, A₁ binds to a polypeptide that is part of a TCR-CD3 complex on the effector cell. In some embodiments, the polypeptide that is part of the TCR-CD3 complex is human CD3ε. In some embodiments, the effector cell antigen comprises CD3, wherein the scFv comprises complementarity determining regions (CDRs): HC-CDR1, HC-CDR2, and HC-CDR3, wherein the HC-CDR1, the HC-CDR2, and the HC-CDR3 of the scFv: HC-CDR1: SEQ ID NO: 1, HC-CDR2: SEQ ID NO: 2, and HC-CDR3: SEQ ID NO: 3; and the scFv comprises CDRs: LC-CDR1, LC-CDR2, and LC-CDR3, wherein the LC-CDR1, the LC-CDR2, and the LC-CDR3 of the scFv LC-CDR1: SEQ ID NO: 4, LC-CDR2: SEQ ID NO:5, and LC-CDR3: SEQ ID NO: 6. In some embodiments, the effector cell antigen comprises CD3, and the scFv comprises an amino acid sequence according to SEQ ID NO: 13. In some embodiments, the effector cell antigen comprises CD3, wherein the scFv comprises complementarity determining regions (CDRs): HC-CDR1, HC-CDR2, and HC-CDR3, wherein the HC-CDR1, the HC-CDR2, and the HC-CDR3 of the scFv: HC-CDR1: SEQ ID NO: 7, HC-CDR2: SEQ ID NO: 8, and HC-CDR3: SEQ ID NO: 9; and the scFv comprises CDRs: LC-CDR1, LC-CDR2, and LC-CDR3, wherein the LC-CDR1, the LC-CDR2, and the LC-CDR3 of the scFv LC-CDR1: SEQ ID NO: 10, LC-CDR2: SEQ ID NO: 11, and LC-CDR3: SEQ ID NO: 12. In some embodiments, the effector cell antigen comprises CD3, and the scFv comprises an amino acid sequence according to SEQ ID NO: 14.

In some embodiments, A₁ comprises an amino acid sequence according to SEQ ID NO: 13. In some embodiments, A₁ comprises an amino acid sequence that has at least 80% sequence identity to SEQ ID NO: 13. In some embodiments, A₁ comprises an amino acid sequence that has at least 85% sequence identity to SEQ ID NO: 13. In some embodiments, A₁ comprises an amino acid sequence that has at least 90% sequence identity to SEQ ID NO: 13. In some embodiments, A₁ comprises an amino acid sequence that has at least 95% sequence identity to SEQ ID NO: 13. In some embodiments, A₁ comprises an amino acid sequence that has at least 99% sequence identity to SEQ ID NO: 13.

In some embodiments, A₁ comprises an amino acid sequence according to SEQ ID NO: 14. In some embodiments, A₁ comprises an amino acid sequence that has at least 80% sequence identity to SEQ ID NO: 14. In some embodiments, A₁ comprises an amino acid sequence that has at least 85% sequence identity to SEQ ID NO: 14. In some embodiments, A₁ comprises an amino acid sequence that has at least 90% sequence identity to SEQ ID NO: 14. In some embodiments, A₁ comprises an amino acid sequence that has at least 95% sequence identity to SEQ ID NO: 14. In some embodiments, A₁ comprises an amino acid sequence that has at least 99% sequence identity to SEQ ID NO: 14.

In some embodiments, the isolated polypeptide or polypeptide complex has weaker binding affinity for the tumor cell antigen as compared to the binding affinity for the tumor cell antigen of an isolated polypeptide or polypeptide complex that does not have P₁ or L₁. In some embodiments, the isolated polypeptide or polypeptide complex has weaker binding affinity for the tumor cell antigen that is at least 5× higher than the binding affinity for the tumor cell antigen of a form of the isolated polypeptide or polypeptide complex that does not have P₁ or L₁. In some embodiments, the isolated polypeptide or polypeptide complex has weaker binding affinity for the tumor cell antigen that is at least 8× higher than the binding affinity for the tumor cell antigen of a form of the isolated polypeptide or polypeptide complex that does not have P₁ or L₁. In some embodiments, the isolated polypeptide or polypeptide complex has weaker binding affinity for the tumor cell antigen that is at least 10× higher than the binding affinity for the tumor cell antigen of a form of the isolated polypeptide or polypeptide complex that does not have P₁ or L₁. In some embodiments, the isolated polypeptide or polypeptide complex has weaker binding affinity for the tumor cell antigen that is at least 15× higher than the binding affinity for the tumor cell antigen of a form of the isolated polypeptide or polypeptide complex that does not have P₁ or L₁. In some embodiments, the isolated polypeptide or polypeptide complex has weaker binding affinity for the tumor cell antigen that is at least 20× higher than the binding affinity for the tumor cell antigen of a form of the isolated polypeptide or polypeptide complex that does not have P₁ or L₁. In some embodiments, the isolated polypeptide or polypeptide complex has weaker binding affinity for the tumor cell antigen that is at least 25× higher than the binding affinity for the tumor cell antigen of a form of the isolated polypeptide or polypeptide complex that does not have P₁ or L₁. In some embodiments, the isolated polypeptide or polypeptide complex has weaker binding affinity for the tumor cell antigen that is at least 30× higher than the binding affinity for the tumor cell antigen of a form of the isolated polypeptide or polypeptide complex that does not have P₁ or L₁. In some embodiments, the isolated polypeptide or polypeptide complex has weaker binding affinity for the tumor cell antigen that is at least 35× higher than the binding affinity for the tumor cell antigen of a form of the isolated polypeptide or polypeptide complex that does not have P₁ or L₁. In some embodiments, the isolated polypeptide or polypeptide complex has weaker binding affinity for the tumor cell antigen that is at least 40× higher than the binding affinity for the tumor cell antigen of a form of the isolated polypeptide or polypeptide complex that does not have P₁ or L₁. In some embodiments, the isolated polypeptide or polypeptide complex has weaker binding affinity for the tumor cell antigen that is at least 45× higher than the binding affinity for the tumor cell antigen of a form of the isolated polypeptide or polypeptide complex that does not have P₁ or L₁. In some embodiments, the isolated polypeptide or polypeptide complex has weaker binding affinity for the tumor cell antigen that is at least 50× higher than the binding affinity for the tumor cell antigen of a form of the isolated polypeptide or polypeptide complex that does not have P₁ or L₁. In some embodiments, the isolated polypeptide or polypeptide complex has weaker binding affinity for the tumor cell antigen that is at least 55× higher than the binding affinity for the tumor cell antigen of a form of the isolated polypeptide or polypeptide complex that does not have P₁ or L₁. In some embodiments, the isolated polypeptide or polypeptide complex has weaker binding affinity for the tumor cell antigen that is at least 60× higher than the binding affinity for the tumor cell antigen of a form of the isolated polypeptide or polypeptide complex that does not have P₁ or L₁. In some embodiments, the isolated polypeptide or polypeptide complex has weaker binding affinity for the tumor cell antigen that is at least 65× higher than the binding affinity for the tumor cell antigen of a form of the isolated polypeptide or polypeptide complex that does not have P₁ or L₁. In some embodiments, the isolated polypeptide or polypeptide complex has weaker binding affinity for the tumor cell antigen that is at least 70× higher than the binding affinity for the tumor cell antigen of a form of the isolated polypeptide or polypeptide complex that does not have P₁ or L₁. In some embodiments, the isolated polypeptide or polypeptide complex has weaker binding affinity for the tumor cell antigen that is at least 75× higher than the binding affinity for the tumor cell antigen of a form of the isolated polypeptide or polypeptide complex that does not have P₁ or L₁. In some embodiments, the isolated polypeptide or polypeptide complex has weaker binding affinity for the tumor cell antigen that is at least 80× higher than the binding affinity for the tumor cell antigen of a form of the isolated polypeptide or polypeptide complex that does not have P₁ or L₁. In some embodiments, the isolated polypeptide or polypeptide complex has weaker binding affinity for the tumor cell antigen that is at least 85× higher than the binding affinity for the tumor cell antigen of a form of the isolated polypeptide or polypeptide complex that does not have P₁ or L₁. In some embodiments, the isolated polypeptide or polypeptide complex has weaker binding affinity for the tumor cell antigen that is at least 90× higher than the binding affinity for the tumor cell antigen of a form of the isolated polypeptide or polypeptide complex that does not have P₁ or L₁. In some embodiments, the isolated polypeptide or polypeptide complex has weaker binding affinity for the tumor cell antigen that is at least 95× higher than the binding affinity for the tumor cell antigen of a form of the isolated polypeptide or polypeptide complex that does not have P₁ or L₁. In some embodiments, the isolated polypeptide or polypeptide complex has weaker binding affinity for the tumor cell antigen that is at least 100× higher than the binding affinity for the tumor cell antigen of a form of the isolated polypeptide or polypeptide complex that does not have P₁ or L₁. In some embodiments, the isolated polypeptide or polypeptide complex has weaker binding affinity for the tumor cell antigen that is at least 120× higher than the binding affinity for the tumor cell antigen of a form of the isolated polypeptide or polypeptide complex that does not have P₁ or L₁. In some embodiments, the isolated polypeptide or polypeptide complex has weaker binding affinity for the tumor cell antigen that is at least 1000× higher than the binding affinity for the tumor cell antigen of a form of the isolated polypeptide or polypeptide complex that does not have P₁ or L₁.

In some embodiments, the isolated polypeptide or polypeptide complex has weaker binding affinity for the tumor cell antigen as compared to the binding affinity for the tumor cell antigen of the isolated polypeptide or polypeptide complex in which L₁ has been cleaved by the tumor specific protease. In some embodiments, the isolated polypeptide or polypeptide complex has weaker binding affinity for the tumor cell antigen that is at least 5× higher than the binding affinity for the tumor cell antigen of the isolated polypeptide or polypeptide complex in which L₁ has been cleaved by the tumor specific protease. In some embodiments, the isolated polypeptide or polypeptide complex has weaker binding affinity for the tumor cell antigen that is at least 8× higher than the binding affinity for the tumor cell antigen of the isolated polypeptide or polypeptide complex in which L₁ has been cleaved by the tumor specific protease. In some embodiments, the isolated polypeptide or polypeptide complex has weaker binding affinity for the tumor cell antigen that is at least 10× higher than the binding affinity for the tumor cell antigen of the isolated polypeptide or polypeptide complex in which L₁ has been cleaved by the tumor specific protease. In some embodiments, the isolated polypeptide or polypeptide complex has weaker binding affinity for the tumor cell antigen that is at least 15× higher than the binding affinity for the tumor cell antigen of the isolated polypeptide or polypeptide complex in which L₁ has been cleaved by the tumor specific protease. In some embodiments, the isolated polypeptide or polypeptide complex has weaker binding affinity for the tumor cell antigen that is at least 20× higher than the binding affinity for the tumor cell antigen of the isolated polypeptide or polypeptide complex in which L₁ has been cleaved by the tumor specific protease. In some embodiments, the isolated polypeptide or polypeptide complex has weaker binding affinity for the tumor cell antigen that is at least 25× higher than the binding affinity for the tumor cell antigen of the isolated polypeptide or polypeptide complex in which L₁ has been cleaved by the tumor specific protease. In some embodiments, the isolated polypeptide or polypeptide complex has weaker binding affinity for the tumor cell antigen that is at least 30× higher than the binding affinity for the tumor cell antigen of the isolated polypeptide or polypeptide complex in which L₁ has been cleaved by the tumor specific protease. In some embodiments, the isolated polypeptide or polypeptide complex has weaker binding affinity for the tumor cell antigen that is at least 35× higher than the binding affinity for the tumor cell antigen of the isolated polypeptide or polypeptide complex in which L₁ has been cleaved by the tumor specific protease. In some embodiments, the isolated polypeptide or polypeptide complex has weaker binding affinity for the tumor cell antigen that is at least 40× higher than the binding affinity for the tumor cell antigen of the isolated polypeptide or polypeptide complex in which L₁ has been cleaved by the tumor specific protease. In some embodiments, the isolated polypeptide or polypeptide complex has weaker binding affinity for the tumor cell antigen that is at least 45× higher than the binding affinity for the tumor cell antigen of the isolated polypeptide or polypeptide complex in which L₁ has been cleaved by the tumor specific protease. In some embodiments, the isolated polypeptide or polypeptide complex has weaker binding affinity for the tumor cell antigen that is at least 50× higher than the binding affinity for the tumor cell antigen of the isolated polypeptide or polypeptide complex in which L₁ has been cleaved by the tumor specific protease. In some embodiments, the isolated polypeptide or polypeptide complex has weaker binding affinity for the tumor cell antigen that is at least 55× higher than the binding affinity for the tumor cell antigen of the isolated polypeptide or polypeptide complex in which L₁ has been cleaved by the tumor specific protease. In some embodiments, the isolated polypeptide or polypeptide complex has weaker binding affinity for the tumor cell antigen that is at least 60× higher than the binding affinity for the tumor cell antigen of the isolated polypeptide or polypeptide complex in which L₁ has been cleaved by the tumor specific protease. In some embodiments, the isolated polypeptide or polypeptide complex has weaker binding affinity for the tumor cell antigen that is at least 65× higher than the binding affinity for the tumor cell antigen of the isolated polypeptide or polypeptide complex in which L₁ has been cleaved by the tumor specific protease. In some embodiments, the isolated polypeptide or polypeptide complex has weaker binding affinity for the tumor cell antigen that is at least 70× higher than the binding affinity for the tumor cell antigen of the isolated polypeptide or polypeptide complex in which L₁ has been cleaved by the tumor specific protease. In some embodiments, the isolated polypeptide or polypeptide complex has weaker binding affinity for the tumor cell antigen that is at least 75× higher than the binding affinity for the tumor cell antigen of the isolated polypeptide or polypeptide complex in which L₁ has been cleaved by the tumor specific protease. In some embodiments, the isolated polypeptide or polypeptide complex has weaker binding affinity for the tumor cell antigen that is at least 80× higher than the binding affinity for the tumor cell antigen of the isolated polypeptide or polypeptide complex in which L₁ has been cleaved by the tumor specific protease. In some embodiments, the isolated polypeptide or polypeptide complex has weaker binding affinity for the tumor cell antigen that is at least 85× higher than the binding affinity for the tumor cell antigen of the isolated polypeptide or polypeptide complex in which L₁ has been cleaved by the tumor specific protease. In some embodiments, the isolated polypeptide or polypeptide complex has weaker binding affinity for the tumor cell antigen that is at least 90× higher than the binding affinity for the tumor cell antigen of the isolated polypeptide or polypeptide complex in which L₁ has been cleaved by the tumor specific protease. In some embodiments, the isolated polypeptide or polypeptide complex has weaker binding affinity for the tumor cell antigen that is at least 95× higher than the binding affinity for the tumor cell antigen of the isolated polypeptide or polypeptide complex in which L₁ has been cleaved by the tumor specific protease. In some embodiments, the isolated polypeptide or polypeptide complex has weaker binding affinity for the tumor cell antigen that is at least 100× higher than the binding affinity for the tumor cell antigen of the isolated polypeptide or polypeptide complex in which L₁ has been cleaved by the tumor specific protease. In some embodiments, the isolated polypeptide or polypeptide complex has weaker binding affinity for the tumor cell antigen that is at least 120× higher than the binding affinity for the tumor cell antigen of the isolated polypeptide or polypeptide complex in which L₁ has been cleaved by the tumor specific protease. In some embodiments, the isolated polypeptide or polypeptide complex has weaker binding affinity for the tumor cell antigen that is at least 1000× higher than the binding affinity for the tumor cell antigen of the isolated polypeptide or polypeptide complex in which L₁ has been cleaved by the tumor specific protease.

In some embodiments, the isolated polypeptide or polypeptide complex has an increased EC₅₀ in an IFNγ release T-cell activation assay as compared to the EC₅₀ in an IFNγ release T-cell activation assay of an isolated polypeptide or polypeptide complex that does not have P₁ or L₁. In some embodiments, the isolated polypeptide or polypeptide complex has an increased EC₅₀ in an IFNγ release T-cell activation assay that is at least 10× higher than the EC₅₀ in an IFNγ release T-cell activation assay of a form of the isolated polypeptide or polypeptide complex that does not have P₁ or L₁. In some embodiments, the isolated polypeptide or polypeptide complex has an increased EC₅₀ in an IFNγ release T-cell activation assay that is at least 20× higher than the EC₅₀ in an IFNγ release T-cell activation assay of a form of the isolated polypeptide or polypeptide complex that does not have P₁ or L₁. In some embodiments, the isolated polypeptide or polypeptide complex has an increased EC₅₀ in an IFNγ release T-cell activation assay that is at least 30× higher than the EC₅₀ in an IFNγ release T-cell activation assay of a form of the isolated polypeptide or polypeptide complex that does not have P₁ or L₁. In some embodiments, the isolated polypeptide or polypeptide complex has an increased EC₅₀ in an IFNγ release T-cell activation assay that is at least 40× higher than the EC₅₀ in an IFNγ release T-cell activation assay of a form of the isolated polypeptide or polypeptide complex that does not have P₁ or L₁. In some embodiments, the isolated polypeptide or polypeptide complex has an increased EC₅₀ in an IFNγ release T-cell activation assay that is at least 50× higher than the EC₅₀ in an IFNγ release T-cell activation assay of a form of the isolated polypeptide or polypeptide complex that does not have P₁ or L₁. In some embodiments, the isolated polypeptide or polypeptide complex has an increased EC₅₀ in an IFNγ release T-cell activation assay that is at least 60× higher than the EC₅₀ in an IFNγ release T-cell activation assay of a form of the isolated polypeptide or polypeptide complex that does not have P₁ or L₁. In some embodiments, the isolated polypeptide or polypeptide complex has an increased EC₅₀ in an IFNγ release T-cell activation assay that is at least 70× higher than the EC₅₀ in an IFNγ release T-cell activation assay of a form of the isolated polypeptide or polypeptide complex that does not have P₁ or L₁. In some embodiments, the isolated polypeptide or polypeptide complex has an increased EC₅₀ in an IFNγ release T-cell activation assay that is at least 80× higher than the EC₅₀ in an IFNγ release T-cell activation assay of a form of the isolated polypeptide or polypeptide complex that does not have P₁ or L₁. In some embodiments, the isolated polypeptide or polypeptide complex has an increased EC₅₀ in an IFNγ release T-cell activation assay that is at least 90× higher than the EC₅₀ in an IFNγ release T-cell activation assay of a form of the isolated polypeptide or polypeptide complex that does not have P₁ or L₁. In some embodiments, the isolated polypeptide or polypeptide complex has an increased EC₅₀ in an IFNγ release T-cell activation assay that is at least 100× higher than the EC₅₀ in an IFNγ release T-cell activation assay of a form of the isolated polypeptide or polypeptide complex that does not have P₁ or L₁. In some embodiments, the isolated polypeptide or polypeptide complex has an increased EC₅₀ in an IFNγ release T-cell activation assay that is at least 1000× higher than the EC₅₀ in an IFNγ release T-cell activation assay of a form of the isolated polypeptide or polypeptide complex that does not have P₁ or L₁.

In some embodiments, the isolated polypeptide or polypeptide complex has an increased EC₅₀ in an IFNγ release T-cell activation assay as compared to the EC₅₀ in an IFNγ release T-cell activation assay of the isolated polypeptide or polypeptide complex in which L₁ has been cleaved by the tumor specific protease. In some embodiments, the isolated polypeptide or polypeptide complex has an increased EC₅₀ in an IFNγ release T-cell activation assay that is at least 10× higher than the EC₅₀ in an IFNγ release T-cell activation assay of the isolated polypeptide or polypeptide complex in which L₁ has been cleaved by the tumor specific protease. In some embodiments, the isolated polypeptide or polypeptide complex has an increased EC₅₀ in an IFNγ release T-cell activation assay that is at least 20× higher than the EC₅₀ in an IFNγ release T-cell activation assay of the isolated polypeptide or polypeptide complex in which L₁ has been cleaved by the tumor specific protease. In some embodiments, the isolated polypeptide or polypeptide complex has an increased EC₅₀ in an IFNγ release T-cell activation assay that is at least 30× higher than the EC₅₀ in an IFNγ release T-cell activation assay of the isolated polypeptide or polypeptide complex in which L₁ has been cleaved by the tumor specific protease. In some embodiments, the isolated polypeptide or polypeptide complex has an increased EC₅₀ in an IFNγ release T-cell activation assay that is at least 40× higher than the EC₅₀ in an IFNγ release T-cell activation assay of the isolated polypeptide or polypeptide complex in which L₁ has been cleaved by the tumor specific protease. In some embodiments, the isolated polypeptide or polypeptide complex has an increased EC₅₀ in an IFNγ release T-cell activation assay that is at least 50× higher than the EC₅₀ in an IFNγ release T-cell activation assay of the isolated polypeptide or polypeptide complex in which L₁ has been cleaved by the tumor specific protease. In some embodiments, the isolated polypeptide or polypeptide complex has an increased EC₅₀ in an IFNγ release T-cell activation assay that is at least 60× higher than the EC₅₀ in an IFNγ release T-cell activation assay of the isolated polypeptide or polypeptide complex in which L₁ has been cleaved by the tumor specific protease. In some embodiments, the isolated polypeptide or polypeptide complex has an increased EC₅₀ in an IFNγ release T-cell activation assay that is at least 70× higher than the EC₅₀ in an IFNγ release T-cell activation assay of the isolated polypeptide or polypeptide complex in which L₁ has been cleaved by the tumor specific protease. In some embodiments, the isolated polypeptide or polypeptide complex has an increased EC₅₀ in an IFNγ release T-cell activation assay that is at least 80× higher than the EC₅₀ in an IFNγ release T-cell activation assay of the isolated polypeptide or polypeptide complex in which L₁ has been cleaved by the tumor specific protease. In some embodiments, the isolated polypeptide or polypeptide complex has an increased EC₅₀ in an IFNγ release T-cell activation assay that is at least 90× higher than the EC₅₀ in an IFNγ release T-cell activation assay of the isolated polypeptide or polypeptide complex in which L₁ has been cleaved by the tumor specific protease. In some embodiments, the isolated polypeptide or polypeptide complex has an increased EC₅₀ in an IFNγ release T-cell activation assay that is at least 100× higher than the EC₅₀ in an IFNγ release T-cell activation assay of the isolated polypeptide or polypeptide complex in which L₁ has been cleaved by the tumor specific protease. In some embodiments, the isolated polypeptide or polypeptide complex has an increased EC₅₀ in an IFNγ release T-cell activation assay that is at least 1000× higher than the EC₅₀ in an IFNγ release T-cell activation assay of the isolated polypeptide or polypeptide complex in which L₁ has been cleaved by the tumor specific protease.

In some embodiments, the isolated polypeptide or polypeptide complex has an increased EC₅₀ in a T-cell cytolysis assay as compared to the EC₅₀ in a T-cell cytolysis assay of an isolated polypeptide or polypeptide complex that does not have P₁ or L₁. In some embodiments, the isolated polypeptide or polypeptide complex has an increased EC₅₀ in a T-cell cytolysis assay that is at least 10× higher than the EC₅₀ in a T-cell cytolysis assay of a form of the isolated polypeptide or polypeptide complex that does not have P₁ or L₁. In some embodiments, the isolated polypeptide or polypeptide complex has an increased EC₅₀ in a T-cell cytolysis assay that is at least 20× higher than the EC₅₀ in a T-cell cytolysis assay of a form of the isolated polypeptide or polypeptide complex that does not have P₁ or L₁. In some embodiments, the isolated polypeptide or polypeptide complex has an increased EC₅₀ in a T-cell cytolysis assay that is at least 30× higher than the EC₅₀ in a T-cell cytolysis assay of a form of the isolated polypeptide or polypeptide complex that does not have P₁ or L₁. In some embodiments, the isolated polypeptide or polypeptide complex has an increased EC₅₀ in a T-cell cytolysis assay that is at least 40× higher than the EC₅₀ in a T-cell cytolysis assay of a form of the isolated polypeptide or polypeptide complex that does not have P₁ or L₁. In some embodiments, the isolated polypeptide or polypeptide complex has an increased EC₅₀ in a T-cell cytolysis assay that is at least 50× higher than the EC₅₀ in a T-cell cytolysis assay of a form of the isolated polypeptide or polypeptide complex that does not have P₁ or L₁. In some embodiments, the isolated polypeptide or polypeptide complex has an increased EC₅₀ in a T-cell cytolysis assay that is at least 60× higher than the EC₅₀ in a T-cell cytolysis assay of a form of the isolated polypeptide or polypeptide complex that does not have P₁ or L₁. In some embodiments, the isolated polypeptide or polypeptide complex has an increased EC₅₀ in a T-cell cytolysis assay that is at least 70× higher than the EC₅₀ in a T-cell cytolysis assay of a form of the isolated polypeptide or polypeptide complex that does not have P₁ or L₁. In some embodiments, the isolated polypeptide or polypeptide complex has an increased EC₅₀ in a T-cell cytolysis assay that is at least 80× higher than the EC₅₀ in a T-cell cytolysis assay of a form of the isolated polypeptide or polypeptide complex that does not have P₁ or L₁. In some embodiments, the isolated polypeptide or polypeptide complex has an increased EC₅₀ in a T-cell cytolysis assay that is at least 90× higher than the EC₅₀ in a T-cell cytolysis assay of a form of the isolated polypeptide or polypeptide complex that does not have P₁ or L₁. In some embodiments, the isolated polypeptide or polypeptide complex has an increased EC₅₀ in a T-cell cytolysis assay that is at least 100× higher than the EC₅₀ in a T-cell cytolysis assay of an isolated polypeptide or polypeptide complex that does not have P₁ or L₁. In some embodiments, the isolated polypeptide or polypeptide complex has an increased EC₅₀ in a T-cell cytolysis assay that is at least 1000× higher than the EC₅₀ in a T-cell cytolysis assay of an isolated polypeptide or polypeptide complex that does not have P₁ or L₁.

In some embodiments, the isolated polypeptide or polypeptide complex has an increased EC₅₀ in a T-cell cytolysis assay as compared to the EC₅₀ in a T-cell cytolysis assay of the isolated polypeptide or polypeptide complex in which L₁ has been cleaved by the tumor specific protease. In some embodiments, the isolated polypeptide or polypeptide complex has an increased EC₅₀ in a T-cell cytolysis assay that is at least 10× higher than the EC₅₀ in a T-cell cytolysis assay of the isolated polypeptide or polypeptide complex in which L₁ has been cleaved by the tumor specific protease. In some embodiments, the isolated polypeptide or polypeptide complex has an increased EC₅₀ in a T-cell cytolysis assay that is at least 20× higher than the EC₅₀ in a T-cell cytolysis assay of the isolated polypeptide or polypeptide complex in which L₁ has been cleaved by the tumor specific protease. In some embodiments, the isolated polypeptide or polypeptide complex has an increased EC₅₀ in a T-cell cytolysis assay that is at least 30× higher than the EC₅₀ in a T-cell cytolysis assay of the isolated polypeptide or polypeptide complex in which L₁ has been cleaved by the tumor specific protease. In some embodiments, the isolated polypeptide or polypeptide complex has an increased EC₅₀ in a T-cell cytolysis assay that is at least 40× higher than the EC₅₀ in a T-cell cytolysis assay of the isolated polypeptide or polypeptide complex in which L₁ has been cleaved by the tumor specific protease. In some embodiments, the isolated polypeptide or polypeptide complex has an increased EC₅₀ in a T-cell cytolysis assay that is at least 50× higher than the EC₅₀ in a T-cell cytolysis assay of the isolated polypeptide or polypeptide complex in which L₁ has been cleaved by the tumor specific protease. In some embodiments, the isolated polypeptide or polypeptide complex has an increased EC₅₀ in a T-cell cytolysis assay that is at least 60× higher than the EC₅₀ in a T-cell cytolysis assay of the isolated polypeptide or polypeptide complex in which L₁ has been cleaved by the tumor specific protease. In some embodiments, the isolated polypeptide or polypeptide complex has an increased EC₅₀ in a T-cell cytolysis assay that is at least 70× higher than the EC₅₀ in a T-cell cytolysis assay of the isolated polypeptide or polypeptide complex in which L₁ has been cleaved by the tumor specific protease. In some embodiments, the isolated polypeptide or polypeptide complex has an increased EC₅₀ in a T-cell cytolysis assay that is at least 80× higher than the EC₅₀ in a T-cell cytolysis assay of the isolated polypeptide or polypeptide complex in which L₁ has been cleaved by the tumor specific protease. In some embodiments, the isolated polypeptide or polypeptide complex has an increased EC₅₀ in a T-cell cytolysis assay that is at least 90× higher than the EC₅₀ in a T-cell cytolysis assay of the isolated polypeptide or polypeptide complex in which L₁ has been cleaved by the tumor specific protease. In some embodiments, the isolated polypeptide or polypeptide complex has an increased EC₅₀ in a T-cell cytolysis assay that is at least 100× higher than the EC₅₀ in a T-cell cytolysis assay of the isolated polypeptide or polypeptide complex in which L₁ has been cleaved by the tumor specific protease. In some embodiments, the isolated polypeptide or polypeptide complex has an increased EC₅₀ in a T-cell cytolysis assay that is at least 1,000× higher than the EC₅₀ in a T-cell cytolysis assay of the isolated polypeptide or polypeptide complex in which L₁ has been cleaved by the tumor specific protease.

In some embodiments, the isolated polypeptide or polypeptide complex P₂-L₂-A₂-A₁-L₁-P₁-H₁ (Formula Ia) has weaker binding affinity for the tumor cell antigen as compared to the binding affinity for the tumor cell antigen of an isolated polypeptide or polypeptide complex of Formula Ia that does not have P₁, L₁, P₂, or L₂. In some embodiments, the isolated polypeptide or polypeptide complex P₂-L₂-A₂-A₁-L₁-P₁—H₁ (Formula Ia) has weaker binding affinity for the tumor cell antigen that is at least 10× higher than the binding affinity for the tumor cell antigen of a form of the isolated polypeptide or polypeptide complex of Formula Ia that does not have P₁, L₁, P₂, or L₂. In some embodiments, the isolated polypeptide or polypeptide complex P₂-L₂-A₂-A₁-L₁-P₁-H₁ (Formula Ia) has weaker binding affinity for the tumor cell antigen that is at least 50× higher than the binding affinity for the tumor cell antigen of a form of the isolated polypeptide or polypeptide complex of Formula Ia that does not have P₁, L₁, P₂, or L₂. In some embodiments, the isolated polypeptide or polypeptide complex P₂-L₂-A₂-A₁-L₁-P₁-H₁ (Formula Ia) has weaker binding affinity for the tumor cell antigen that is at least 75× higher than the binding affinity for the tumor cell antigen of a form of the isolated polypeptide or polypeptide complex of Formula Ia that does not have P₁, L₁, P₂, or L₂. In some embodiments, the isolated polypeptide or polypeptide complex P₂-L₂-A₂-A₁-L₁-P₁-H₁(Formula Ia) has weaker binding affinity for the tumor cell antigen that is at least 100× higher than the binding affinity for the tumor cell antigen of a form of the isolated polypeptide or polypeptide complex of Formula Ia that does not have P₁, L₁, P₂, or L₂. In some embodiments, the isolated polypeptide or polypeptide complex P₂-L₂-A₂-A₁-L₁-P₁-H₁ (Formula Ia) has weaker binding affinity for the tumor cell antigen that is at least 120× higher than the binding affinity for the tumor cell antigen of a form of the isolated polypeptide or polypeptide complex of Formula Ia that does not have P₁, L₁, P₂, or L₂. In some embodiments, the isolated polypeptide or polypeptide complex P₂-L₂-A₂-A₁-L₁-P₁-H₁ (Formula Ia) has weaker binding affinity for the tumor cell antigen that is at least 200× higher than the binding affinity for the tumor cell antigen of a form of the isolated polypeptide or polypeptide complex of Formula Ia that does not have P₁, L₁, P₂, or L₂. In some embodiments, the isolated polypeptide or polypeptide complex P₂-L₂-A₂-A₁-L₁-P₁-H₁ (Formula Ia) has weaker binding affinity for the tumor cell antigen that is at least 300× higher than the binding affinity for the tumor cell antigen of a form of the isolated polypeptide or polypeptide complex of Formula Ia that does not have P₁, L₁, P₂, or L₂. In some embodiments, the isolated polypeptide or polypeptide complex P₂-L₂-A₂-A₁-L₁-P₁-H₁ (Formula Ia) has weaker binding affinity for the tumor cell antigen that is at least 400× higher than the binding affinity for the tumor cell antigen of a form of the isolated polypeptide or polypeptide complex of Formula Ia that does not have P₁, L₁, P₂, or L₂. In some embodiments, the isolated polypeptide or polypeptide complex P₂-L₂-A₂-A₁-L₁-P₁-H₁ (Formula Ia) has weaker binding affinity for the tumor cell antigen that is at least 500× higher than the binding affinity for the tumor cell antigen of a form of the isolated polypeptide or polypeptide complex of Formula Ia that does not have P₁, L₁, P₂, or L₂. In some embodiments, the isolated polypeptide or polypeptide complex P₂-L₂-A₂-A₁-L₁-P₁-H₁ (Formula Ia) has weaker binding affinity for the tumor cell antigen that is at least 600× higher than the binding affinity for the tumor cell antigen of a form of the isolated polypeptide or polypeptide complex of Formula Ia that does not have P₁, L₁, P₂, or L₂. In some embodiments, the isolated polypeptide or polypeptide complex P₂-L₂-A₂-A₁-L₁-P₁-H₁ (Formula Ia) has weaker binding affinity for the tumor cell antigen that is at least 700× higher than the binding affinity for the tumor cell antigen of a form of the isolated polypeptide or polypeptide complex of Formula Ia that does not have P₁, L₁, P₂, or L₂. In some embodiments, the isolated polypeptide or polypeptide complex P₂-L₂-A₂-A₁-L₁-P₁-H₁ (Formula Ia) has weaker binding affinity for the tumor cell antigen that is at least 800× higher than the binding affinity for the tumor cell antigen of a form of the isolated polypeptide or polypeptide complex of Formula Ia that does not have P₁, L₁, P₂, or L₂. In some embodiments, the isolated polypeptide or polypeptide complex P₂-L₂-A₂-A₁-L₁-P₁-H₁ (Formula Ia) has weaker binding affinity for the tumor cell antigen that is at least 900× higher than the binding affinity for the tumor cell antigen of a form of the isolated polypeptide or polypeptide complex of Formula Ia that does not have P₁, L₁, P₂, or L₂. In some embodiments, the isolated polypeptide or polypeptide complex P₂-L₂-A₂-A₁-L₁-P₁-H₁ (Formula Ia) has weaker binding affinity for the tumor cell antigen that is at least 1000× higher than the binding affinity for the tumor cell antigen of a form of the isolated polypeptide or polypeptide complex of Formula Ia that does not have P₁, L₁, P₂, or L₂. In some embodiments, the isolated polypeptide or polypeptide complex P₂-L₂-A₂-A₁-L₁-P₁-H₁ (Formula Ia) has weaker binding affinity for the tumor cell antigen that is at least 10,000× higher than the binding affinity for the tumor cell antigen of a form of the isolated polypeptide or polypeptide complex of Formula Ia that does not have P₁, L₁, P₂, or L₂.

In some embodiments, the isolated polypeptide or polypeptide complex P₂-L₂-A₂-A₁-L₁-P₁-H₁ (Formula Ia) has weaker binding affinity for the tumor cell antigen as compared to the binding affinity for the tumor cell antigen of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has weaker binding affinity for the tumor cell antigen that is at least 10× higher than the binding affinity for the tumor cell antigen of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁(Formula Ia) has weaker binding affinity for the tumor cell antigen that is at least 50× higher than the binding affinity for the tumor cell antigen of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has weaker binding affinity for the tumor cell antigen that is at least 75× higher than the binding affinity for the tumor cell antigen of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁(Formula Ia) has weaker binding affinity for the tumor cell antigen that is at least 100× higher than the binding affinity for the tumor cell antigen of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has weaker binding affinity for the tumor cell antigen that is at least 120× higher than the binding affinity for the tumor cell antigen of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁(Formula Ia) has weaker binding affinity for the tumor cell antigen that is at least 200× higher than the binding affinity for the tumor cell antigen of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has weaker binding affinity for the tumor cell antigen that is at least 300× higher than the binding affinity for the tumor cell antigen of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁(Formula Ia) has weaker binding affinity for the tumor cell antigen that is at least 400× higher than the binding affinity for the tumor cell antigen of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has weaker binding affinity for the tumor cell antigen that is at least 500× higher than the binding affinity for the tumor cell antigen of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁(Formula Ia) has weaker binding affinity for the tumor cell antigen that is at least 600× higher than the binding affinity for the tumor cell antigen of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has weaker binding affinity for the tumor cell antigen that is at least 700× higher than the binding affinity for the tumor cell antigen of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁(Formula Ia) has weaker binding affinity for the tumor cell antigen that is at least 800× higher than the binding affinity for the tumor cell antigen of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has weaker binding affinity for the tumor cell antigen that is at least 900× higher than the binding affinity for the tumor cell antigen of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁(Formula Ia) has weaker binding affinity for the tumor cell antigen that is at least 1000× higher than the binding affinity for the tumor cell antigen of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has weaker binding affinity for the tumor cell antigen that is at least 10,000× higher than the binding affinity for the tumor cell antigen of the isolated polypeptide or polypeptide complex of Formula Ia in which L₁ and L₂ have been cleaved by the tumor specific proteases.

In some embodiments, the isolated polypeptide or polypeptide complex P₂-L₂-A₂-A₁-L₁-P₁-H₁ (Formula Ia) has an increased EC₅₀ in an IFNγ release T-cell activation assay as compared to the EC₅₀ in an IFNγ release T-cell activation assay of an isolated polypeptide or polypeptide complex of Formula Ia that does not have P₁, L₁, P₂, or L₂. In some embodiments, the isolated polypeptide or polypeptide complex P₂-L₂-A₂-A₁-L₁-P₁-H₁ (Formula Ia) has an increased EC₅₀ in an IFNγ release T-cell activation assay that is at least 10× higher than the EC₅₀ in an IFNγ release T-cell activation assay of a form of the isolated polypeptide or polypeptide complex of Formula Ia that does not have P₁, L₁, P₂, or L₂. In some embodiments, the isolated polypeptide or polypeptide complex P₂-L₂-A₂-A₁-L₁-P₁-H₁ (Formula Ia) has an increased EC₅₀ in an IFNγ release T-cell activation assay that is at least 50× higher than the EC₅₀ in an IFNγ release T-cell activation assay of a form of the isolated polypeptide or polypeptide complex of Formula Ia that does not have P₁, L₁, P₂, or L₂. In some embodiments, the isolated polypeptide or polypeptide complex P₂-L₂-A₂-A₁-L₁-P₁-H₁ (Formula Ia) has an increased EC₅₀ in an IFNγ release T-cell activation assay that is at least 75× higher than the EC₅₀ in an IFNγ release T-cell activation assay of a form of the isolated polypeptide or polypeptide complex of Formula Ia that does not have P₁, L₁, P₂, or L₂. In some embodiments, the isolated polypeptide or polypeptide complex P₂-L₂-A₂-A₁-L₁-P₁-H₁ (Formula Ia) has an increased EC₅₀ in an IFNγ release T-cell activation assay that is at least 100× higher than the EC₅₀ in an IFNγ release T-cell activation assay of a form of the isolated polypeptide or polypeptide complex of Formula Ia that does not have P1, L₁, P₂, or L₂. In some embodiments, the isolated polypeptide or polypeptide complex P₂-L₂-A₂-A₁-L₁-P₁-H₁ (Formula Ia) has an increased EC₅₀ in an IFNγ release T-cell activation assay that is at least 200× higher than the EC₅₀ in an IFNγ release T-cell activation assay of a form of the isolated polypeptide or polypeptide complex of Formula Ia that does not have P1, L₁, P₂, or L₂. In some embodiments, the isolated polypeptide or polypeptide complex P₂-L₂-A₂-A₁-L₁-P₁-H₁ (Formula Ia) has an increased EC₅₀ in an IFNγ release T-cell activation assay that is at least 300× higher than the EC₅₀ in an IFNγ release T-cell activation assay of a form of the isolated polypeptide or polypeptide complex of Formula Ia that does not have P1, L₁, P₂, or L₂. In some embodiments, the isolated polypeptide or polypeptide complex P₂-L₂-A₂-A₁-L₁-P₁-H₁ (Formula Ia) has an increased EC₅₀ in an IFNγ release T-cell activation assay that is at least 400× higher than the EC₅₀ in an IFNγ release T-cell activation assay of a form of the isolated polypeptide or polypeptide complex of Formula Ia that does not have P1, L₁, P₂, or L₂. In some embodiments, the isolated polypeptide or polypeptide complex P₂-L₂-A₂-A₁-L₁-P₁-H₁ (Formula Ia) has an increased EC₅₀ in an IFNγ release T-cell activation assay that is at least 500× higher than the EC₅₀ in an IFNγ release T-cell activation assay of a form of the isolated polypeptide or polypeptide complex of Formula Ia that does not have P1, L₁, P₂, or L₂. In some embodiments, the isolated polypeptide or polypeptide complex P₂-L₂-A₂-A₁-L₁-P₁-H₁ (Formula Ia) has an increased EC₅₀ in an IFNγ release T-cell activation assay that is at least 600× higher than the EC₅₀ in an IFNγ release T-cell activation assay of a form of the isolated polypeptide or polypeptide complex of Formula Ia that does not have P1, L₁, P₂, or L₂. In some embodiments, the isolated polypeptide or polypeptide complex P₂-L₂-A₂-A₁-L₁-P₁-H₁ (Formula Ia) has an increased EC₅₀ in an IFNγ release T-cell activation assay that is at least 700× higher than the EC₅₀ in an IFNγ release T-cell activation assay of a form of the isolated polypeptide or polypeptide complex of Formula Ia that does not P1, L₁, P₂, or L₂. In some embodiments, the isolated polypeptide or polypeptide complex P₂-L₂-A₂-A₁-L₁-P₁-H₁ (Formula Ia) has an increased EC₅₀ in an IFNγ release T-cell activation assay that is at least 800× higher than the EC₅₀ in an IFNγ release T-cell activation assay of a form of the isolated polypeptide or polypeptide complex of Formula Ia that does not have P₁, L₁, P₂, or L₂. In some embodiments, the isolated polypeptide or polypeptide complex P₂-L₂-A₂-A₁-L₁-P₁-H₁ (Formula Ia) has an increased EC₅₀ in an IFNγ release T-cell activation assay that is at least 900× higher than the EC₅₀ in an IFNγ release T-cell activation assay of a form of the isolated polypeptide or polypeptide complex of Formula Ia that does not have P1, L₁, P₂, or L₂. In some embodiments, the isolated polypeptide or polypeptide complex P₂-L₂-A₂-A₁-L₁-P₁-H₁ (Formula Ia) has an increased EC₅₀ in an IFNγ release T-cell activation assay that is at least 1000× higher than the EC₅₀ in an IFNγ release T-cell activation assay of a form of the isolated polypeptide or polypeptide complex of Formula Ia that does not have P₁, L₁, P₂, or L₂. In some embodiments, the isolated polypeptide or polypeptide complex P₂-L₂-A₂-A₁-L₁-P₁-H₁ (Formula Ia) has an increased EC₅₀ in an IFNγ release T-cell activation assay that is at least 10,000× higher than the EC₅₀ in an IFNγ release T-cell activation assay of a form of the isolated polypeptide or polypeptide complex of Formula Ia that does not have P1, L₁, P₂, or L₂.

In some embodiments, the isolated polypeptide or polypeptide complex P₂-L₂-A₂-A₁-L₁-P₁-H₁ (Formula Ia) has an increased EC₅₀ in a T-cell cytolysis assay as compared to the EC₅₀ in a T-cell cytolysis assay of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 10× higher than the EC₅₀ in a T-cell cytolysis assay of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 50× higher than the EC₅₀ in a T-cell cytolysis assay of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 75× higher than the EC₅₀ in a T-cell cytolysis assay of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 100× higher than the EC₅₀ in a T-cell cytolysis assay of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 200× higher than the EC₅₀ in a T-cell cytolysis assay of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 300× higher than the EC₅₀ in a T-cell cytolysis assay of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 400× higher than the EC₅₀ in a T-cell cytolysis assay of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 500× higher than the EC₅₀ in a T-cell cytolysis assay of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 600× higher than the EC₅₀ in a T-cell cytolysis assay of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 700× higher than the EC₅₀ in a T-cell cytolysis assay of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 800× higher than the EC₅₀ in a T-cell cytolysis assay of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 900× higher than the EC₅₀ in a T-cell cytolysis assay of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 1000× higher than the EC₅₀ in a T-cell cytolysis assay of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 10,000× higher than the EC₅₀ in a T-cell cytolysis assay of the isolated polypeptide or polypeptide complex of Formula Ia in which L₁ and L₂ have been cleaved by the tumor specific proteases.

In some embodiments, the isolated polypeptide or polypeptide complex P₂-L₂-A₂-A₁-L₁-P₁-H₁ (Formula Ia) has an increased EC₅₀ in a T-cell cytolysis assay as compared to the EC₅₀ in a T-cell cytolysis assay of an isolated polypeptide or polypeptide complex that does not have P1, L₁, P₂, or L₂. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 10× higher than the EC₅₀ in a T-cell cytolysis assay of a form of the isolated polypeptide or polypeptide complex of Formula Ia that does not have P₁, L₁, P₂, or L₂. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 50× higher than the EC₅₀ in a T-cell cytolysis assay of a form of the isolated polypeptide or polypeptide complex of Formula Ia that does not have P1, L₁, P₂, or L₂. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 75× higher than the EC₅₀ in a T-cell cytolysis assay of a form of the isolated polypeptide or polypeptide complex of Formula Ia that does not have P1, L₁, P₂, or L₂. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 100× higher than the EC₅₀ in a T-cell cytolysis assay of an isolated polypeptide or polypeptide complex that does not have P1, L₁, P₂, or L₂. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 200× higher than the EC₅₀ in a T-cell cytolysis assay of an isolated polypeptide or polypeptide complex that does not have Pi, L₁, P₂, or L₂. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 300× higher than the EC₅₀ in a T-cell cytolysis assay of an isolated polypeptide or polypeptide complex that does not have P₁, L₁, P₂, or L₂. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 400× higher than the EC₅₀ in a T-cell cytolysis assay of an isolated polypeptide or polypeptide complex that does not have P₁, L₁, P₂, or L₂. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 500× higher than the EC₅₀ in a T-cell cytolysis assay of an isolated polypeptide or polypeptide complex that does not have P₁, L₁, P₂, or L₂. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 600× higher than the EC₅₀ in a T-cell cytolysis assay of an isolated polypeptide or polypeptide complex that does not have P₁, L₁, P₂, or L₂. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 700× higher than the EC₅₀ in a T-cell cytolysis assay of an isolated polypeptide or polypeptide complex that does not have P₁, L₁, P₂, or L₂. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 800× higher than the EC₅₀ in a T-cell cytolysis assay of an isolated polypeptide or polypeptide complex that does not have P₁, L₁, P₂, or L₂. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 900× higher than the EC₅₀ in a T-cell cytolysis assay of an isolated polypeptide or polypeptide complex that does not have P₁, L₁, P₂, or L₂. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 1000× higher than the EC₅₀ in a T-cell cytolysis assay of an isolated polypeptide or polypeptide complex that does not have P₁, L₁, P₂, or L₂. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 10,000× higher than the EC₅₀ in a T-cell cytolysis assay of an isolated polypeptide or polypeptide complex that does not have P₁, L₁, P₂, or L₂.

In some embodiments, the isolated polypeptide or polypeptide complex P₂-L₂-A₂-A₁-L₁-P₁-H₁ (Formula Ia) has an increased EC₅₀ in a T-cell cytolysis assay as compared to the EC₅₀ in a T-cell cytolysis assay of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 10× higher than the EC₅₀ in a T-cell cytolysis assay of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 50× higher than the EC₅₀ in a T-cell cytolysis assay of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 75× higher than the EC₅₀ in a T-cell cytolysis assay of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 100× higher than the EC₅₀ in a T-cell cytolysis assay of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 200× higher than the EC₅₀ in a T-cell cytolysis assay of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 300× higher than the EC₅₀ in a T-cell cytolysis assay of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 400× higher than the EC₅₀ in a T-cell cytolysis assay of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 500× higher than the EC₅₀ in a T-cell cytolysis assay of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 600× higher than the EC₅₀ in a T-cell cytolysis assay of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 700× higher than the EC₅₀ in a T-cell cytolysis assay of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 800× higher than the EC₅₀ in a T-cell cytolysis assay of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 900× higher than the EC₅₀ in a T-cell cytolysis assay of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 1000× higher than the EC₅₀ in a T-cell cytolysis assay of the isolated polypeptide or polypeptide complex of Formula Ia in which L₁ and L₂ have been cleaved by the tumor specific proteases. In some embodiments, the isolated polypeptide or polypeptide complex P₂-L₂-A₂-A₁-L₁-P₁-H₁ (Formula Ia) has an increased EC₅₀ in a T-cell cytolysis assay that is at least 10,000× higher than the EC₅₀ in a T-cell cytolysis assay of the isolated polypeptide or polypeptide complex of Formula Ia in which L₁ and L₂ have been cleaved by the tumor specific proteases.

### Second Antigen Recognizing Molecule (A₂)

In some embodiments, A₂ comprises an antibody or antibody fragment. In some embodiments, the antibody or antibody fragment thereof comprises a single chain variable fragment, a single domain antibody, a Fab, or a Fab′. In some embodiments, the antibody or antibody fragment thereof comprises a single chain variable fragment (scFv), a heavy chain variable domain (VH domain), a light chain variable domain (VL domain), a variable domain (VHH) of a camelid derived single domain antibody. In some embodiments, the antibody or antibody fragment thereof is humanized or human. In some embodiments, A₂ is the Fab or Fab′. In some embodiments, the Fab or Fab′ comprises (a) a Fab light chain polypeptide and (b) a Fab heavy chain polypeptide. In some embodiments, the antibody or antibody fragment thereof comprises a TROP2 binding domain.

In some embodiments, the antigen binding fragment (Fab) or Fab′ that binds to TROP2 comprises a Fab light chain polypeptide chain and a Fab heavy chain polypeptide. In some embodiments, the Fab light chain polypeptide comprises a Fab light chain variable domain. In some embodiments, the Fab heavy chain polypeptide comprises a Fab heavy chain variable domain. In some embodiments, the Fab heavy chain variable domain comprises at least one, two, or three complementarity determining regions (CDR)s disclosed in Table 2 or a sequence substantially identical thereto (e.g., a sequence that has at least 90%, 95%, 96%, 97%, 98%, or 99% sequence identity). In some embodiments, the Fab light chain variable domain comprises at least one, two, or three complementarity determining regions (CDR)s disclosed in Table 2 or a sequence substantially identical thereto (e.g., a sequence that has at least 90%, 95%, 96%, 97%, 98%, or 99% sequence identity).

In some embodiments, the Fab heavy chain variable domain comprises at least one, two, or three complementarity determining regions (CDR)s disclosed in Table 2 or a sequence substantially identical thereto (e.g., a sequence that has at least 90%, 95%, 96%, 97%, 98%, or 99% sequence identity); and the Fab light chain variable domain comprises at least one, two, or three complementarity determining regions (CDR)s disclosed in Table 2 or a sequence substantially identical thereto (e.g., a sequence that has at least 90%, 95%, 96%, 97%, 98%, or 99% sequence identity).

TABLE 2
anti-TROP2 amino acid sequences (CDRs as determined by IMGT numbering system)

| | Amino Acid Sequence | SEQ ID |
|---|---|---|
| Construct Description | (N to C) | NO: |
| TROP2: HC: CDR1 | GYTFTNYG | 15 |
| TROP2: HC: CDR2 | INTYTGEP | 16 |
| TROP2: HC: CDR3 | ARGGFGSSYWYFDV | 17 |
| TROP2: LC: CDR1 | QDVSIA | 18 |
| TROP2: LC: CDR2 | SAS | 19 |
| TROP2: LC: CDR3 | QQHYITPLT | 20 |
| TROP2 Fab LC | DIQLTQSPSSLSASVGDRVSITCKA | 21 |
| | SQDVSIAVAWYQQKPGKAPKLLI | |
| | YSASYRYTGVPDRFSGSGSGTDFT | |
| | LTISSLQPEDFAVYYCQQHYITPL | |
| | TFGAGTKVEIKRTVAAPSVFIFPPS | |
| | DEQLKSGTASVVCLLNNFYPREA | |
| | KVQWKVDNALQSGNSQESVTEQ | |
| | DSKDSTYSLSSTLTLSKADYEKHK | |
| | VYACEVTHQGLSSPVTKSFNRGE | |
| | C | |
| TROP2 Fab HC | QVQLQQSGSELKKPGASVKVSCK | 22 |
| | ASGYTFTNYGMNWVKQAPGQG | |
| | LKWMGWINTYTGEPTYTDDFKG | |
| | RFAFSLDTSVSTAYLQISSLKADD | |
| | TAVYFCARGGFGSSYWYFDVW | |
| | GQGSLVTVSSASTKGPSVFPLAPS | |
| | SKSTSGGTAALGCLVKDYFPEPV | |
| | TVSWNSGALTSGVHTFPAVLQSS | |
| | GLYSLSSVVTVPSSSLGTQTYICN | |
| | VNHKPSNTKVDKKVEPKSC | |

In some embodiments, the Fab comprises complementarity determining regions (CDRs): HC-CDR1, HC-CDR2, and HC-CDR3, wherein the HC-CDR1, the HC-CDR2, and the HC-CDR3 of the Fab comprise: HC-CDR11: SEQ ID NO: 15, HC-CDR2: SEQ ID NO: 16, and HC-CDR3: SEQ ID NO: 17; and the Fab comprises CDRs: LC-CDR1, LC-CDR2, and LC-CDR3, wherein the LC-CDR1, the LC-CDR2, and the LC-CDR3 of the Fab comprise LC-CDR1: SEQ ID NO: 18, LC-CDR2: SEQ ID NO: 19, and LC-CDR3: SEQ ID NO: 20. In some embodiments, the Fab comprises complementarity determining regions (CDRs): HC-CDR1, HC-CDR2, and HC-CDR3, wherein the HC-CDR1, the HC-CDR2, and the HC-CDR3 of the Fab comprise: HC-CDR1: SEQ ID NO: 15, HC-CDR2: SEQ ID NO: 16, and HC-CDR3: SEQ ID NO: 17 and wherein the CDRs comprise from 0-2 amino acid modifications in at least one of the HC-CDR1, HC-CDR2, or HC-CDR3; and the Fab comprises CDRs: LC-CDR1, LC-CDR2, and LC-CDR3, wherein the LC-CDR1, the LC-CDR2, and the LC-CDR3 of the Fab comprise LC-CDR11: SEQ ID NO: 18, LC-CDR2: SEQ ID NO: 19, and LC-CDR3: SEQ ID NO: 20 and wherein the CDRs comprise from 0-2 amino acid modifications in at least one of the LC-CDR1, LC-CDR2, or LC-CDR3.

In some embodiments, A₂ comprises complementarity determining regions (CDRs): HC-CDR1, HC-CDR2, and HC-CDR3, wherein the HC-CDR1, the HC-CDR2, and the HC-CDR3 of A₂ comprise: HC-CDR1: SEQ ID NO: 15, HC-CDR2: SEQ ID NO: 16, and HC-CDR3: SEQ ID NO: 17; and A₂ comprises CDRs: LC-CDR1, LC-CDR2, and LC-CDR3, wherein the LC-CDR1, the LC-CDR2, and the LC-CDR3 of A₂ comprise LC-CDR1: SEQ ID NO: 18, LC-CDR2: SEQ ID NO: 19, and LC-CDR3: SEQ ID NO: 20.

In some embodiments, the Fab light chain polypeptide comprises an amino acid sequence according to SEQ ID NO: 21. In some embodiments, the Fab light chain polypeptide comprises an amino acid sequence that has at least 80% sequence identity according to SEQ ID NO: 21. In some embodiments, the Fab light chain polypeptide comprises an amino acid sequence that has at least 85% sequence identity according to SEQ ID NO: 21. In some embodiments, the Fab light chain polypeptide comprises an amino acid sequence that has at least 90% sequence identity according to SEQ ID NO: 21. In some embodiments, the Fab light chain polypeptide comprises an amino acid sequence that has at least 95% sequence identity according to SEQ ID NO: 21. In some embodiments, the Fab light chain polypeptide comprises an amino acid sequence that has at least 99% sequence identity according to SEQ ID NO: 21.

In some embodiments, the Fab heavy chain polypeptide comprises an amino acid sequence according to SEQ ID NO: 22. In some embodiments, the Fab heavy chain polypeptide comprises an amino acid sequence that has at least 80% sequence identity according to SEQ ID NO: 22. In some embodiments, the Fab heavy chain polypeptide comprises an amino acid sequence that has at least 85% sequence identity according to SEQ ID NO: 22. In some embodiments, the Fab heavy chain polypeptide comprises an amino acid sequence that has at least 90% sequence identity according to SEQ ID NO: 22. In some embodiments, the Fab heavy chain polypeptide comprises an amino acid sequence that has at least 95% sequence identity according to SEQ ID NO: 22. In some embodiments, the Fab heavy chain polypeptide comprises an amino acid sequence that has at least 99% sequence identity according to SEQ ID NO: 22.

In some embodiments, the Fab light chain polypeptide of A₂ is bound to a C-terminus of the single chain variable fragment (scFv) of A₁. In some embodiments, the Fab heavy chain polypeptide of A₂ is bound to a C-terminus of the single chain variable fragment (scFv) A₁. In some embodiments, the Fab light chain polypeptide of A₂ is bound to a N-terminus of the single chain variable fragment (scFv) of A₁. In some embodiments, the Fab heavy chain polypeptide of A₂ is bound to a N-terminus of the single chain variable fragment (scFv) A₁. In some embodiments, the Fab heavy chain polypeptide of A₂ is bound to the scFv heavy chain polypeptide of A₁. In some embodiments, Fab light chain polypeptide of A₂ is bound to the scFv heavy chain polypeptide of A₁. In some embodiments, the Fab heavy chain polypeptide of A₂ is bound to the scFv light chain polypeptide of A₁. In some embodiments, the Fab light chain polypeptide of A₂ is bound to the scFv light chain polypeptide of A₁.

In some embodiments, A₂ further comprises P₂ and L₂, wherein P₂ comprises a peptide that binds to A₂; and L₂ comprises a linking moiety that connects A₂ to P₂ and is a substrate for a tumor specific protease. In some embodiments, the Fab heavy chain polypeptide of A₂ is bound to the scFv heavy chain polypeptide of A₁ and L₂ is bound to the Fab light chain polypeptide of A₂. In some embodiments, the Fab light chain polypeptide of A₂ is bound to the scFv heavy chain polypeptide of A₁ and L₂ is bound to the Fab heavy chain polypeptide of A₂. In some embodiments, the Fab heavy chain polypeptide of A₂ is bound to the scFv light chain polypeptide of A₁ and L₂ is bound to the Fab light chain polypeptide of A₂. In some embodiments, the Fab light chain polypeptide of A₂ is bound to the scFv light chain polypeptide of A₁ and L₂ is bound to the Fab heavy chain polypeptide of A₂.

In some embodiments, the Fab heavy chain polypeptide of A₂ is bound to the scFv heavy chain polypeptide of A₁ and L₂ is bound to the Fab light chain polypeptide of A₂. In some embodiments, the Fab light chain polypeptide of A₂ is bound to the scFv heavy chain polypeptide of A₁ and L₂ is bound to the Fab heavy chain polypeptide of A₂. In some embodiments, the Fab heavy chain polypeptide of A₂ is bound to the scFv light chain polypeptide of A₁ and L₂ is bound to the Fab light chain polypeptide of A₂. In some embodiments, the Fab light chain polypeptide of A₂ is bound to the scFv light chain polypeptide of A₁ and L₂ is bound to the Fab heavy chain polypeptide of A₂.

Peptide (P₁ and P₂ and P₁ₐ)

In some embodiments, P₁, P₂, or P₁ₐ comprises a sequence as disclosed in Table 3 or a sequence substantially identical thereto (e.g., a sequence that has 0, 1, or 2 amino acid modifications).

TABLE 3
P₁ and P₂ and P₁ₐ Sequences

| | | SEQ |
|---|---|---|
| | Amino Acid | ID |
| Construct Description | Sequence (N to C) | NO: |
| TROP2 Fab Peptide-1 | SVLFCVKNLYCWVT | 23 |
| TROP2 Fab Peptide-2 | VDFCKIYSWPVCHQ | 24 |
| TROP2 Fab Peptide-3 | IDFCMLYNWPICAG | 25 |
| SP34.185 scFv Peptide-A | GSQCLGPEWEVCPY | 26 |
| SP34.185 scFv Peptide-B | VYCGPEFDESVGCM | 27 |
| SP34.194 scFv Peptide-AM | GYLWGCEWNCAGITT | 122 |

In some embodiments, P₁ impairs binding of A₁ to a first target antigen. In some embodiments, P₁ impairs binding of A₁ to the effector cell antigen. In some embodiments, P₁ is bound to A₁ through ionic interactions, electrostatic interactions, hydrophobic interactions, Pi-stacking interactions, and H-bonding interactions, or a combination thereof. In some embodiments, P₁ is bound to A₁ at or near an antigen binding site. In some embodiments, P₁ becomes unbound from A₁ when L₁ is cleaved by the tumor specific protease thereby exposing A₁ to the effector cell antigen. In some embodiments, the protease comprises a matrix metalloprotease (MMP) or a serine protease. In some embodiments, the matrix metalloprotease comprises MMP2, MMP7, MMP9, MMP13, or MMP14. In some embodiments, the serine protease comprises matriptase (MTSP1), urokinase, or hepsin. In some embodiments, P₁ has less than 70% sequence identity to the effector cell antigen. In some embodiments, P₁ has less than 75% sequence identity to the effector cell antigen. In some embodiments, P₁ has less than 80% sequence identity to the effector cell antigen. In some embodiments, P₁ has less than 85% sequence identity to the effector cell antigen. In some embodiments, P₁ has less than 90% sequence identity to the effector cell antigen. In some embodiments, P₁ has less than 95% sequence identity to the effector cell antigen. In some embodiments, P₁ has less than 98% sequence identity to the effector cell antigen. In some embodiments, P₁ has less than 99% sequence identity to the effector cell antigen. In some embodiments, P₁ comprises a de novo amino acid sequence that shares less than 10% sequence identity to the effector cell antigen. In some embodiments, P₁ comprises an amino acid sequence according to SEQ ID NO: 26 or 27. In some embodiments, P₁ comprises the amino acid sequence of SEQ ID NO: 26. In some embodiments, P₁ comprises the amino acid sequence of SEQ ID NO: 27. In some embodiments, P₁ comprises the amino acid sequence of SEQ ID NO: 122.

In some embodiments, P₁ comprises an amino acid sequence according to Z₁-Z₂-C-Z₄-P-Z₆-Z₇-Z₈-Z₉-Z₁₀-Z₁₁-Z₁₂-C-Z₁₄ and Z₁ is selected from D, Y, F, I, N, V, H, L, A, T, S, and P; Z₂ is selected from D, Y, L, F, I, N, A, V, H, T, and S; Z₄ is selected from G and W; Z₆ is selected from E, D, V, and P; Z₇ is selected from W, L, F, V, G, M, I, and Y; Z₈ is selected from E, D, P, and Q; Z₉ is selected from E, D, Y, V, F, W, P, L, and Q; Z₁₀ is selected from S, D, Y, T, I, F, V, N, A, P, L, and H; Z₁₁ is selected from I, Y, F, V, L, T, N, S, D, A, and H; Z₁₂ is selected from F, D, Y, L, I, V, A, N, T, P, S, and H; and Z₁₄ is selected from D, Y, N, F, I, P, V, A, T, H, L and S. In some embodiments, Z₁ is selected from D, Y, F, I, and N; Z₂ is selected from D, Y, L, F, I, and N; Z₄ is selected from G and W; Z₆ is selected from E and D; Z₇ is selected from W, L, F, and V; Z₈ is selected from E and D; Z₉ is selected from E, D, Y, and V; Z₁₀ is selected from S, D, Y, T, and I; Z₁ is selected from I, Y, F, V, L, and T; Z₁₂ is selected from F, D, Y, L, I, V, A, and N; and Z₁₄ is selected from D, Y, N, F, I, and P. In some embodiments, Z₁ is selected from D, Y, and F; Z₂ is selected from D, Y, L, and F; Z₄ is selected from G and W; Z₆ is selected from E and D; Z₇ is selected from W, L, and F; Z₈ is selected from E and D; Z₉ is selected from E and D; Z₁₀ is selected from S, D, and Y; Z₁ is selected from I, Y, and F; Z₁₂ is selected from F, D, Y, and L; and Z₁₄ is selected from D, Y, and N.

In some embodiments, P₁ comprises an amino acid sequence according to U₁-U₂-C-U₄-P-U₆-U₇-U₈-U₉-U₁₀-U₁₁-U₁₂-C-U₁₄ and U₁ is selected from D, Y, F, I, N, V, H, L, A, T, S, and P; U₂ is selected from D, Y, L, F, I, N, A, V, H, T, and S; U₄ is selected from G and W; U₆ is selected from E, D, V, and P; U₇ is selected from W, L, F, V, G, M, I, and Y; U₈ is selected from E, D, P, and Q; U₉ is selected from E, D, Y, V, F, W, P, L, and Q; U₁₀ is selected from S, D, Y, T, I, F, V, N, A, P, L, and H; U₁₁ is selected from I, Y, F, V, L, T, N, S, D, A, and H; U₁₂ is selected from F, D, Y, L, I, V, A, N, T, P, S, G, and H; and U₁₄ is selected from D, Y, N, F, I, P, V, A, T, H, L, M, and S. In some embodiments, U₁ is selected from D, Y, F, I, V, and N; U₂ is selected from D, Y, L, F, I, and N; U₄ is selected from G and W; U₆ is selected from E and D; U₇ is selected from W, L, F, G, and V; U₈ is selected from E and D; U₉ is selected from E, D, Y, and V; U₁₀ is selected from S, D, Y, T, and I; U₁₁ is selected from I, Y, F, V, L, and T; U₁₂ is selected from F, D, Y, L, I, V, A, G, and N; and U₁₄ is selected from D, Y, N, F, I, M, and P. In some embodiments, U₁ is selected from D, Y, V, and F; U₂ is selected from D, Y, L, and F; U₄ is selected from G and W; U₆ is selected from E and D; U₇ is selected from W, L, G, and F; U₈ is selected from E and D; U₉ is selected from E and D; U₁₀ is selected from S, D, T, and Y; U₁₁ is selected from I, Y, V, L, and F; U₁₂ is selected from F, D, Y, G, A, and L; and U₁₄ is selected from D, Y, M, and N.

In some embodiments, P₁ comprises the amino acid sequences according to SEQ ID NOs: 202-228.

In some embodiments, P₁ comprises an amino acid sequences according to any of the sequences of Table 35.

In some embodiments, P₁ comprises the amino acid sequences according to SEQ ID NOs: 229-240.

In some embodiments, P₁ comprises the amino acid sequence according to SEQ ID NO: 239 or a peptide sequence that has 1, 2, or 3, amino acid substitutions, additions, or deletions relative to the amino acid sequence of SEQ ID NO: 239.

In some embodiments, P₁ comprises the amino acid sequence according to SEQ ID NO: 27 or a peptide sequence that has 1, 2, or 3, amino acid substitutions, additions, or deletions relative to the amino acid sequence of SEQ ID NO: 27.

In some embodiments, P₁ comprises the amino acid sequence according to SEQ ID NO: 26 or a peptide sequence that has 1, 2, or 3, amino acid substitutions, additions, or deletions relative to the amino acid sequence of SEQ ID NO: 26.

In some embodiments, P₁ comprises the amino acid sequence according to SEQ ID NO: 239.

In some embodiments, P₁ comprises the amino acid sequence according to SEQ ID NO: 27.

In some embodiments, P₁ comprises the amino acid sequence according to SEQ ID NO: 26.

In some embodiments, P₂ impairs binding of A₂ to a second target antigen. In some embodiments, wherein P₂ impairs binding of A₂ to TROP2. In some embodiments, P₂ is bound to A₂ through ionic interactions, electrostatic interactions, hydrophobic interactions, Pi-stacking interactions, and H-bonding interactions, or a combination thereof. In some embodiments, P₂ is bound to A₂ at or near an antigen binding site. In some embodiments, P₂ becomes unbound from A₂ when L₂ is cleaved by the tumor specific protease thereby exposing A₂ to TROP2. In some embodiments, the protease comprises a matrix metalloprotease (MMP) or a serine protease. In some embodiments, the matrix metalloprotease comprises MMP2, MMP7, MMP9, MMP13, or MMP14. In some embodiments, the serine protease comprises matriptase (MTSP1), urokinase, or hepsin. In some embodiments, P₂ has less than 70% sequence identity to the TROP2. In some embodiments, P₂ has less than 75% sequence identity to TROP2. In some embodiments, P₂ has less than 80% sequence identity to TROP2. In some embodiments, P₂ has less than 85% sequence identity to the TROP2. In some embodiments, P₂ has less than 90% sequence identity to TROP2. In some embodiments, P₂ has less than 95% sequence identity to TROP2. In some embodiments, P₂ has less than 98% sequence identity to TROP2. In some embodiments, P₂ has less than 99% sequence identity to the TROP2. In some embodiments, P₂ comprises a de novo amino acid sequence that shares less than 10% sequence identity to TROP2. In some embodiments, P₂ comprises an amino acid sequence according to any one of SEQ ID NOs: 23-25. In some embodiments, P₂ comprises the amino acid sequence of SEQ ID NO: 23. In some embodiments, P₂ comprises the amino acid sequence of SEQ ID NO: 24. In some embodiments, P₂ comprising the amino acid sequence of SEQ ID NO: 25.

In some embodiments, P₂ comprises an amino acid sequence according to X₁-X₂-X₃-X₄-C-X₆-X₇-X₈-X₉-X₁₀-C-X₁₂-X₁₃-X₁₄ and X₁ is selected from N, D, S, Y, A, F, H, T, L, and V; X₂ is selected from S, T, D, A, H, V, Y, N, F, I, and L; X₃ is selected from L, I, and V; X₄ is selected from F, L, V, M, W, I, Y, and H; X₆ is selected from V, F, L, I, and W; X₇ is selected from K, R, Q, N, H, and M; X₈ is selected from N and K; X₉ is selected from L, V, and I; X₁₀ is selected from Y, W, F, Q, and L; X₁₂ is selected from W and V; X₁₃ is selected from I, N, H, T, V, Y, and D; X₁₄ is selected from D, V, A, S, I, T, N, Y, H, and P. In some embodiments, X₁ is selected from N, D, S, Y, A, F, and T; X₂ is selected from S, T, D, A, H, V, Y, and N; X₃ is L; X₄ is selected from F, L, V, M, and W; X₆ is selected from V, F, and L; X₇ is selected from K, R, Q, and N; X₈ is selected from N and K; X₉ is selected from L, V, and I; X₁₀ is selected from Y, W, and F; X₁₂ is W; X₁₃ is selected from I, N, H, and T; and X₁₄ is selected from D, V, A, S, I, T, and N. In some embodiments, X₁ is selected from N, D, S, and Y; X₂ is selected from S, T, and D; X₃ is L; X₄ is selected from F, L, and V; X₆ is selected from V and F; X₇ is selected from K, R, and Q; X₈ is N; X₉ is selected from L and V; X₁₀ is selected from Y and W; X₁₂ is W; X₁₃ is selected from I, N, and H; X₁₄ is selected from D, V, A, and S.

In some embodiments, P₂ comprises an amino acid sequence according to an amino acid sequence according to J₁-J₂-J₃-C-J₅-J₆-J₇-J₈-W-J₁₀-J₁₁-C-J₁₃-J₁₄ and J₁ is selected V, I, L, P, E, F, and M; J₂ is selected from D and N; J₃ is selected from F and W; J₅ is selected from A, E, S, R, K, Y, L, Q, G, M, F, T, W, and D; J₆ is selected from L, M, I, V, F, T, R, and S; J₇ is selected from Y, F, and N; J₈ is selected from N, R, D, H, K, Q, S, G, A, E, and M; J₁₀ is selected from P and R; J₁₁ is selected from V and I; J₁₃ is selected from D, G, N, R, S, Q, Y, T, A, E, L, V, K, M, I, H, F, and W; and J₁₄ is selected from T, S, Q, L, D, N, A, E, K, M, V, R, I, H, P, V, and W. In some embodiments, J₁ is selected from V, I, and L; J₂ is D; J₃ is F; J₅ is selected from A, E, S, R, K, Y, and L; J₆ is selected from L, M, and I; J₇ is Y; J₈ is selected from N, R, D, H, K, Q, S, and G; J₁₀ is P; J₁ is selected from V and I; J₁₃ is selected from D, G, N, R, S, Q, Y, T, and A; and J₁₄ is selected from T, S, Q, L, D, N, A, and E. In some embodiments, J₁ is selected from V, I, and L; J₂ is D; J₃ is F; J₅ is selected from A, E, S, and R; J₆ is selected from L, M, and I; J₇ is Y; J₈ is selected from N, R, and D; J₁₀ is P; J₁₁ is selected from V and I; J₁₃ is selected from D, G, N, R, S, and Q; and J₁₄ is selected from T, S, Q, and L.

In some embodiments, P₂ comprises an amino acid sequence according to an amino acid sequence according to B₁-B₂-B₃-C-B₅-B₆-B₇-B₈-W-B₁₀-B₁₁-C-B₁₃-B₁₄ and B₁ is selected from V, I, L, P, E, F, and M; B₂ is selected from D and N; B₃ is selected from F and W; B₅ is selected from A, E, S, R, K, Y, L, Q, G, M, F, T, W, and D; B₆ is selected from L, M, I, V, F, T, R, and S; B₇ is selected from Y, F, and N; B₈ is selected from N, R, D, H, K, Q, S, G, A, E, and M; B₁₀ is selected from P and R; B₁₁ is selected from V and I; B₁₃ is selected from D, G, N, R, S, Q, Y, T, A, E, L, V, K, M, I, H, F, and W; and B₁₄ is selected from T, S, Q, L, D, N, A, E, K, M, V, R, I, H, P, V, G, and W. In some embodiments, B₁ is selected from V, I, and L; B₂ is D; B₃ is F; B₅ is selected from A, E, S, R, K, Y, M, G, and L; B₆ is selected from L, M, and I; B₇ is Y; B₈ is selected from N, R, D, H, K, Q, S, and G; B₁₀ is P; B₁₁ is selected from V and I; B₁₃ is selected from D, G, N, R, S, Q, Y, T, H, and A; and B₁₄ is selected from T, S, Q, L, D, N, A, G, and E. In some embodiments, B₁ is selected from V, I, and L; B₂ is D; B₃ is F; B₈ is selected from A, E, S, K, M, G, and R; B₆ is selected from L, M, and I; B₇ is Y; B₈ is selected from N, R, S, H, and D; B₁₀ is P; B₁₁ is selected from V and I; B₁₃ is selected from D, G, N, R, S, H, A, Y, and Q; B₁₄ is selected from T, S, Q, G, and L.

In some embodiments, P₂ comprises the amino acid sequences according to SEQ ID NOs: 133-145.

In some embodiments, P₂ comprises the amino acid sequences according to SEQ ID NOs: 146-158.

In some embodiments, P₂ comprises an amino acid sequence according to any of the sequences of Table 27.

In some embodiments, P₂ comprises the amino acid sequences according to SEQ ID NOs: 159-178.

In some embodiments, P₂ comprises an amino acid sequence according to any of the sequences of Table 29.

In some embodiments, P₂ comprises the amino acid sequences according to SEQ ID NOs: 179-201.

In some embodiments, P₂ comprises the amino acid sequence according to SEQ ID NO: 24 or a peptide sequence that has 1, 2, or 3, amino acid substitutions, additions, or deletions relative to the amino acid sequence of SEQ ID NO: 24.

In some embodiments, P₂ comprises the amino acid sequence according to SEQ ID NO: 181 or a peptide sequence that has 1, 2, or 3, amino acid substitutions, additions, or deletions relative to the amino acid sequence of SEQ ID NO: 181.

In some embodiments, P₂ comprises the amino acid sequence according to SEQ ID NO: 186 or a peptide sequence that has 1, 2, or 3, amino acid substitutions, additions, or deletions relative to the amino acid sequence of SEQ ID NO: 186.

In some embodiments, P₂ comprises the amino acid sequence according to SEQ ID NO: 24.

In some embodiments, P₂ comprises the amino acid sequence according to SEQ ID NO: 181.

In some embodiments, P₂ comprises the amino acid sequence according to SEQ ID NO: 186.

In some embodiments, P₁ₐ when L₁ₐ is uncleaved impairs binding of the first antigen recognizing molecule to the effector cell antigen. In some embodiments, the first antigen recognizing molecule comprises an antibody or antibody fragment. In some embodiments, the effector cell antigen is an anti-CD3 effector cell antigen. In some embodiments, P₁ₐ has less than 70% sequence identity to the effector cell antigen. In some embodiments, P₁ₐ has less than 75% sequence identity to the effector cell antigen. In some embodiments, P₁ₐ has less than 80% sequence identity to the effector cell antigen. In some embodiments, P₁ₐ has less than 85% sequence identity to the effector cell antigen. In some embodiments, P₁ₐ has less than 90% sequence identity to the effector cell antigen. In some embodiments, P₁ₐ has less than 95% sequence identity to the effector cell antigen. In some embodiments, P₁ₐ has less than 98% sequence identity to the effector cell antigen. In some embodiments, P₁ₐ has less than 99% sequence identity to the effector cell antigen. In some embodiments, P₁ₐ comprises a de novo amino acid sequence that shares less than 10% sequence identity to the second effector cell antigen. In some embodiments, P₁ₐ comprises an amino acid sequence according to SEQ ID NO: 26 or 27 or 122.

In some embodiments, P₁ₐ comprises an amino acid sequence according to Z₁-Z₂-C-Z₄-P-Z₆-Z₇-Z₈—Z₉-Z₁₀-Z₁₁-Z₁₂-C-Z₁₄ and Z₁ is selected from D, Y, F, I, N, V, H, L, A, T, S, and P; Z₂ is selected from D, Y, L, F, I, N, A, V, H, T, and S; Z₄ is selected from G and W; Z₆ is selected from E, D, V, and P; Z₇ is selected from W, L, F, V, G, M, I, and Y; Z₈ is selected from E, D, P, and Q; Z₉ is selected from E, D, Y, V, F, W, P, L, and Q; Z₁₀ is selected from S, D, Y, T, I, F, V, N, A, P, L, and H; Z₁₁ is selected from I, Y, F, V, L, T, N, S, D, A, and H; Z₁₂ is selected from F, D, Y, L, I, V, A, N, T, P, S, and H; and Z₁₄ is selected from D, Y, N, F, I, P, V, A, T, H, L and S. In some embodiments, Z₁ is selected from D, Y, F, I, and N; Z₂ is selected from D, Y, L, F, I, and N; Z₄ is selected from G and W; Z₆ is selected from E and D; Z₇ is selected from W, L, F, and V; Z₈ is selected from E and D; Z₉ is selected from E, D, Y, and V; Z₁₀ is selected from S, D, Y, T, and I; Z₁ is selected from I, Y, F, V, L, and T; Z₁₂ is selected from F, D, Y, L, I, V, A, and N; and Z₁₄ is selected from D, Y, N, F, I, and P. In some embodiments, Z₁ is selected from D, Y, and F; Z₂ is selected from D, Y, L, and F; Z₄ is selected from G and W; Z₆ is selected from E and D; Z₇ is selected from W, L, and F; Z₈ is selected from E and D; Z₉ is selected from E and D; Z₁₀ is selected from S, D, and Y; Z₁ is selected from I, Y, and F; Z₁₂ is selected from F, D, Y, and L; and Z₁₄ is selected from D, Y, and N.

In some embodiments, P₁ₐ comprises an amino acid sequence according to U₁-U₂-C-U₄-P-U₆-U₇—U₈-U₉-U₁₀-U₁₁-U₁₂-C-U₁₄ and U₁ is selected from D, Y, F, I, N, V, H, L, A, T, S, and P; U₂ is selected from D, Y, L, F, I, N, A, V, H, T, and S; U₄ is selected from G and W; U₆ is selected from E, D, V, and P; U₇ is selected from W, L, F, V, G, M, I, and Y; U₈ is selected from E, D, P, and Q; U₉ is selected from E, D, Y, V, F, W, P, L, and Q; U₁₀ is selected from S, D, Y, T, I, F, V, N, A, P, L, and H; U₁₁ is selected from I, Y, F, V, L, T, N, S, D, A, and H; U₁₂ is selected from F, D, Y, L, I, V, A, N, T, P, S, G, and H; and U₁₄ is selected from D, Y, N, F, I, P, V, A, T, H, L, M, and S. In some embodiments, U₁ is selected from D, Y, F, I, V, and N; U₂ is selected from D, Y, L, F, I, and N; U₄ is selected from G and W; U₆ is selected from E and D; U₇ is selected from W, L, F, G, and V; U₈ is selected from E and D; U₉ is selected from E, D, Y, and V; U₁₀ is selected from S, D, Y, T, and I; U₁₁ is selected from I, Y, F, V, L, and T; U₁₂ is selected from F, D, Y, L, I, V, A, G, and N; and U₁₄ is selected from D, Y, N, F, I, M, and P. In some embodiments, U₁ is selected from D, Y, V, and F; U₂ is selected from D, Y, L, and F; U₄ is selected from G and W; U₆ is selected from E and D; U₇ is selected from W, L, G, and F; U₈ is selected from E and D; U₉ is selected from E and D; U₁₀ is selected from S, D, T, and Y; U₁₁ is selected from I, Y, V, L, and F; U₁₂ is selected from F, D, Y, G, A, and L; and U₁₄ is selected from D, Y, M, and N.

In some embodiments, P₁ₐ comprises the amino acid sequences according to SEQ ID NOs: 202-228.

In some embodiments, P₁ₐ comprises an amino acid sequence according to any of the sequences of Table 35.

In some embodiments, P₁ₐ comprises the amino acid sequences according to SEQ ID NOs: 229-240.

In some embodiments, P₁ₐ comprises the amino acid sequence according to SEQ ID NO: 239 or a peptide sequence that has 1, 2, or 3, amino acid substitutions, additions, or deletions relative to the amino acid sequence of SEQ ID NO: 239.

In some embodiments, P₁ₐ comprises the amino acid sequence according to SEQ ID NO: 27 or a peptide sequence that has 1, 2, or 3, amino acid substitutions, additions, or deletions relative to the amino acid sequence of SEQ ID NO: 27.

In some embodiments, P₁ₐ comprises the amino acid sequence according to SEQ ID NO: 26 or a peptide sequence that has 1, 2, or 3, amino acid substitutions, additions, or deletions relative to the amino acid sequence of SEQ ID NO: 26.

In some embodiments, P₁ₐ comprises the amino acid sequence according to SEQ ID NO: 239.

In some embodiments, P₁ₐ comprises the amino acid sequence according to SEQ ID NO: 27.

In some embodiments, P₁ₐ comprises the amino acid sequence according to SEQ ID NO: 26.

In some embodiments, P₁, P₂, or P₁ₐ comprises a peptide sequence of at least 5 amino acids in length. In some embodiments, P₁, P₂, or P₁ₐ comprises a peptide sequence of at least 6 amino acids in length. In some embodiments, P₁, P₂, or P₁ₐ comprises a peptide sequence of at least 10 amino acids in length. In some embodiments, P₁, P₂, or P₁ₐ comprises a peptide sequence of at least 10 amino acids in length and no more than 20 amino acids in length. In some embodiments, P₁, P₂, or P₁ₐ comprises a peptide sequence of at least 16 amino acids in length. In some embodiments, P₁, P₂, or P₁ₐ comprises a peptide sequence of no more than 40 amino acids in length. In some embodiments, P₁, P₂, or P₁ₐ comprises at least two cysteine amino acid residues. In some embodiments, P₁, P₂, or P₁ₐ comprises a cyclic peptide or a linear peptide. In some embodiments, P₁, P₂, or P₁ₐ comprises a cyclic peptide. In some embodiments, P₁, P₂, or P₁ₐ comprises a linear peptide.

In some embodiments, P₁, P₂, or P₁ₐ or P₁, P₂, and P₁ₐ comprise a modified amino acid or non-natural amino acid, or a modified non-natural amino acid, or a combination thereof. In some embodiments, the modified amino acid or a modified non-natural amino acid comprises a post-translational modification. In some embodiments P₁, P₂, or P₁ₐ or P₁, P₂, and P₁ₐ comprise a modification including, but not limited to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to P₁, P₂, or P₁ₐ or P₁, P₂, and P₁ₐ including the peptide backbone, the amino acid side chains, and the terminus.

In some embodiments, P₁, P₂, or P₁ₐ does not comprise albumin or an albumin fragment. In some embodiments, P₁, P₂, or P₁ₐ does not comprise an albumin binding domain.

Linking Moiety (L₁, L₂, L₃, and L₁ₐ)

In some embodiments, L₁, L₂, L₃, or L₁ₐ is a peptide sequence having at least 5 to no more than 50 amino acids. In some embodiments L₁, L₂, L₃, or L₁ₐ is a peptide sequence having at least 10 to no more than 30 amino acids. In some embodiments, L₁, L₂, L₃, or L₁ₐ is a peptide sequence having at least 10 amino acids. In some embodiments, L₁, L₂, L₃, or L₁ₐ is a peptide sequence having at least 18 amino acids. In some embodiments, L₁, L₂, L₃, or L₁ₐ is a peptide sequence having at least 26 amino acids. In some embodiments, L₁, L₂, L₃, or L₁ₐ has a formula comprising (G₂S)ₙ (SEQ ID NO: 243), wherein n is an integer from 1 to 3. In some embodiments, L₁, L₂, L₃, or L₁ₐ has a formula comprising (G₂S)ₙ, wherein n is an integer of at least 1. In some embodiments, L₁, L₂, L₃, or L₁ₐ has a formula selected from the group consisting of (G₂S)ₙ, (GS)ₙ, (GSGGS)ₙ (SEQ ID NO: 62), (GGGS)ₙ (SEQ ID NO: 63), (GGGGS)ₙ (SEQ ID NO: 64), and (GSSGGS)ₙ (SEQ ID NO: 65), wherein n is an integer of at least 1. In some embodiments, the tumor specific protease is selected from the group consisting of metalloprotease, serine protease, cysteine protease, threonine protease, and aspartic protease. In some embodiments L₁, L₂, L₃, or L₁ₐ comprises a urokinase cleavable amino acid sequence, a matriptase cleavable amino acid sequence, a legumain cleavable amino acid sequence, or a matrix metalloprotease cleavable amino acid sequence. In some embodiments, the protease comprises a matrix metalloprotease (MMP) or a serine protease. In some embodiments, the matrix metalloprotease comprises MMP2, MMP7, MMP9, MMP13, or MMP14. In some embodiments, the serine protease comprises matriptase (MTSP1), urokinase, or hepsin.

In some embodiments, L₁, L₂, L₃, or L₁ₐ comprises a sequence as disclosed in Table 4 or a sequence substantially identical thereto (e.g., a sequence that has 0, 1, or 2 amino acid modifications).

In some embodiments, L₁, comprises the sequence of Linker-25 (SEQ ID NO: 54). In some embodiments, L₁, comprises the sequence of Linker-26 (SEQ ID NO: 55). In some embodiments, L₁, comprises the sequence of Linker-27 (SEQ ID NO: 56). In some embodiments, L₁, comprises the sequence of Linker-28 (SEQ ID NO: 57).

In some embodiments, L₂, comprises the sequence of Linker-25 (SEQ ID NO: 54). In some embodiments, L₂, comprises the sequence of Linker-26 (SEQ ID NO: 55). In some embodiments, L₂, comprises the sequence of Linker-27 (SEQ ID NO: 56). In some embodiments, L₂, comprises the sequence of Linker-28 (SEQ ID NO: 57).

TABLE 4
L₁, L₂, L₃, and L₁ₐ Sequences

| | | SEQ |
|---|---|---|
| Construct | Amino Acid Sequence | ID |
| Description | (N to C) | NO: |
| Linker 1 | GGGGSGGGGSGGGGS | 28 |
| Linker 2 | GGGGS | 29 |
| Linker 3 | GGGGSGGGS | 30 |
| Cleavable | GGGGSGGGLSGRSDAGSPLGL | 31 |
| linker 1 | AGSGGGS | |
| Cleavable | GGGGSGGLSGRSDAGSPLGLA | 32 |
| linker 2 | GSGGS | |
| Linker 4 | GGGGSLSGRSDNHGSSGT | 33 |
| Linker 5 | GGGGSSGGSGGSGLSGRSDNH | 34 |
| | GSSGT | |
| Linker 6 | ASGRSDNH | 35 |
| Linker 7 | LAGRSDNH | 36 |
| Linker 8 | ISSGLASGRSDNH | 37 |
| Linker 9 | ISSGLLAGRSDNH | 38 |
| Linker 10 | LSGRSDNH | 39 |
| Linker 11 | ISSGLLSGRSDNP | 40 |
| Linker 12 | ISSGLLSGRSDNH | 41 |
| Linker 13 | LSGRSDNHSPLGLAGS | 42 |
| Linker 14 | SPLGLAGSLSGRSDNH | 43 |
| Linker 15 | SPLGLSGRSDNH | 44 |
| Linker 16 | LAGRSDNHSPLGLAGS | 45 |
| Linker 17 | LSGRSDNHVPLSLKMG | 46 |
| Linker 18 | LSGRSDNHVPLSLSMG | 47 |
| Linker 19 | GSSGGSGGSGGSGISSGLLSGR | 48 |
| | SDNHGSSGT | |
| Linker 20 | GSSGGSGGSGGISSGLLSGRSD | 49 |
| | NHGGGS | |
| Linker 21 | ASGRSDNH | 50 |
| Linker 22 | LAGRSDNH | 51 |
| Linker 23 | ISSGLASGRSDNH | 52 |
| Linker 24 | LSGRSDAG | 53 |
| Linker 25 | ISSGLLSGRSDAG | 54 |
| Linker 26 | AAGLLAPPGGLSGRSDAG | 55 |
| Linker 27 | SPLGLSGRSDAG | 56 |
| Linker 28 | LSGRSDAGSPLGLAG | 57 |
| Cleavable | GGGGSSGGSAAGLLAPPGGLS | 58 |
| linker 3 | GRSDAGGGGS | |
| Cleavable | GSSGGSAAGLLAPPGGLSGRS | 59 |
| linker 4 | DAGGGGS | |
| Non-cleavable | GGGGSGGGGGSGGGGSGGAS | 60 |
| linker 1 | SGAGGSGGS | |
| Non-cleavable | GGGGSGGGSGGGGSGGASSG | 61 |
| linker 2 | AGGSGGGS | |

In some embodiments, L₁ is bound to N-terminus of A₁. In some embodiments, L₁ is bound to C-terminus of A₁. In some embodiments, L₂ is bound to N-terminus of A₂. In some embodiments, L₂ is bound to C-terminus of A₂. In some embodiments, P₁ becomes unbound from A₁ when L₁ is cleaved by the tumor specific protease thereby exposing A₁ to the effector cell antigen. In some embodiments, P₂ becomes unbound from A₂ when L₂ is cleaved by the tumor specific protease thereby exposing A₂ to TROP2.

In some embodiments, L₁, L₂, L₃, or L₁ₐ comprise a modified amino acid or non-natural amino acid, or a modified non-natural amino acid, or a combination thereof. In some embodiments, the modified amino acid or a modified non-natural amino acid comprises a post-translational modification. In some embodiments, L₁, L₂, L₃, or L₁ₐ comprise a modification including, but not limited, to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to L₁, L₂, L₃, or L₁ₐ including the peptide backbone, or the amino acid side chains.

In some embodiments, the cleavable linker is cleavable by a protease. In some embodiments, the protease is present in higher levels in a disease-state microenvironment relative to levels in healthy tissue or a microenvironment that is not the disease-state microenvironment. In some embodiments, the protease comprises a tumor specific protease. In some embodiments, the protease comprises a matrix metalloprotease (MMP) or a serine protease. In some embodiments, the matrix metalloprotease comprises MMP2, MMP7, MMP9, MMP13, or MMP14. In some embodiments, the matrix metalloprotease is selected from the group consisting of MMP2, MMP7, MMP9, MMP13, and MMP14. In some embodiments, the matrix metalloprotease comprises MMP2. In some embodiments, the matrix metalloprotease comprises MMP7. In some embodiments, the matrix metalloprotease comprises MMP9. In some embodiments, the matrix metalloprotease comprises MMP13. In some embodiments, the matrix metalloprotease comprises MMP14. In some embodiments, the serine protease comprises matriptase (MTSP1), urokinase, or hepsin. In some embodiments, the serine protease is selected from the group consisting of matriptase (MTSP1), urokinase, and hepsin. In some embodiments, the serine protease comprises matriptase (MTSP1). In some embodiments, the serine protease comprises urokinase. In some embodiments, the serine protease comprises hepsin. In some embodiments, the cleavable linker is cleaved by a variety of proteases. In some embodiments, the cleavable linker is cleaved by at least 1, 2, 3, 4, 5, 6, 7, 8, 9, 10, 15, 20, or more than 20 different proteases.

Half-Life Extending Molecule (H₁ and H₁ₐ)

In some embodiments, H₁ does not block A₁ binding to the effector cell antigen. In some embodiments, H₁ comprises a linking moiety (L₃) that connects H₁ to P₁. In some embodiments, H₁ₐ does not block the first antigen recognizing molecule binding to the effector cell antigen. In some embodiments, H₁ₐ comprises a linking moiety (L₃) that connects H₁ₐ to P₁ₐ. In some embodiments, the half-life extending molecule (H₁ or H₁ₐ) does not have binding affinity to the first antigen recognizing molecule. In some embodiments, the half-life extending molecule (H₁ or H₁ₐ) does not have binding affinity to the effector cell antigen. In some embodiments, the half-life extending molecule (H₁ or H₁ₐ) does not shield the first antigen recognizing molecule from the effector cell antigen. In some embodiments, the half-life extending molecule (H₁ or H₁ₐ) is not directly linked to the first antigen recognizing molecule.

In some embodiments, H₁ or H₁ₐ comprises a sequence as disclosed in Table 5 or a sequence substantially identical thereto (e.g., a sequence that has at least 90%, 95%, 96%, 97%, 98%, or 99% sequence identity).

TABLE 5
H₁ and H₁ₐ Sequences

| | Amino Acid Sequence | |
|---|---|---|
| Construct Description | (N to C) | SEQ ID NO: |
| Anti-Albumin: CDR-H1 | GSTFYTAV | 66 |
| Anti-Albumin: CDR-H2 | IRWTALTT | 67 |
| Anti-Albumin: CDR-H3 | AARGTLGLFTTADSYDY | 68 |
| Anti-albumin | EVQLVESGGGLVQPGGSLRLSCAASGSTF | 69 |
| | YTAVMGWVRQAPGKGLEWVAAIRWTA | |
| | LTTSYADSVKGRFTISRDGAKTTLYLQM | |
| | NSLRPEDTAVYYCAARGTLGLFTTADSY | |
| | DYWGQGTLVTVSS | |
| 10G Anti-Albumin: CDR-H1 | GFTFSKFG | 70 |
| 10G Anti-Albumin: CDR-H2 | ISGSGRDT | 71 |
| 10G Anti-Albumin: CDR-H3 | TIGGSLSV | 72 |
| 10G Anti-albumin | EVQLVESGGGLVQPGNSLRLSCAASGFT | 73 |
| | FSKFGMSWVRQAPGKGLEWVSSISGSGR | |
| | DTLYADSVKGRFTISRDNAKTTLYLQMN | |
| | SLRPEDTAVYYCTIGGSLSVSSQGTLVTV | |
| | SS | |

In some embodiments, H₁ or H₁ₐ comprise an amino acid sequence that has repetitive sequence motifs. In some embodiments, H₁ or H₁ₐ comprises an amino acid sequence that has highly ordered secondary structure. “Highly ordered secondary structure,” as used in this context, means that at least about 50%, or about 70%, or about 80%, or about 90%, of amino acid residues of H₁ or H₁ₐ contribute to secondary structure, as measured or determined by means, including, but not limited to, spectrophotometry (e.g. by circular dichroism spectroscopy in the “far-UV” spectral region (190-250 nm), and computer programs or algorithms, such as the Chou-Fasman algorithm and the Gamier-Osguthorpe-Robson (“GOR”) algorithm.

In some embodiments, H₁ or H₁ₐ comprises a polymer. In some embodiments, the polymer is polyethylene glycol (PEG). In some embodiments, H₁ or H₁ₐ comprises albumin. In some embodiments, H₁ or H₁ₐ comprises an Fc domain. In some embodiments, the albumin is serum albumin. In some embodiments, the albumin is human serum albumin. In some embodiments, H₁ or H₁ₐ comprises a polypeptide, a ligand, or a small molecule. In some embodiments, the polypeptide, the ligand or the small molecule binds serum protein or a fragment thereof, a circulating immunoglobulin or a fragment thereof, or CD35/CR1. In some embodiments, the serum protein comprises a thyroxine-binding protein, a transthyretin, a 1-acid glycoprotein, a transferrin, transferrin receptor or a transferrin-binding portion thereof, a fibrinogen, or an albumin. In some embodiments, the circulating immunoglobulin molecule comprises IgG1, IgG2, IgG3, IgG4, slgA, IgM or IgD. In some embodiments, the serum protein is albumin. In some embodiments, the polypeptide is an antibody. In some embodiments, the antibody comprises a single domain antibody, a single chain variable fragment or a Fab. In some embodiments, the single domain antibody comprises a single domain antibody that binds to albumin. In some embodiments, the single domain antibody is a human or humanized antibody. In some embodiments, the single domain antibody is selected from the group consisting of 645gH1gL1, 645dsgH5gL4, 23-13-A01-sc02, A10m3 or a fragment thereof, DOM7r-31, DOM7h-11-15, Alb-1, Alb-8, Alb-23, 10G, 10E and SA21. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 66, HC-CDR2: SEQ ID NO: 67, and HC-CDR3: SEQ ID NO: 68. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 66, HC-CDR2: SEQ ID NO: 67, and HC-CDR3: SEQ ID NO: 68; and wherein the CDRs comprise from 0-2 amino acid modifications in at least one of the HC-CDR1, HC-CDR2, or HC-CDR3. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 70, HC-CDR2: SEQ ID NO: 71, and HC-CDR3: SEQ ID NO: 72. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 70, HC-CDR2: SEQ ID NO: 71, and HC-CDR3: SEQ ID NO: 72; and wherein the CDRs comprise from 0-2 amino acid modifications in at least one of the HC-CDR1, HC-CDR2, or HC-CDR3.

In some embodiments, H₁ comprises an amino acid sequence according to SEQ ID NO: 69. In some embodiments, H₁ comprises an amino acid sequence that has at least 80% sequence identity to SEQ ID NO: 69. In some embodiments, H₁ comprises an amino acid sequence that has at least 85% sequence identity to SEQ ID NO: 69. In some embodiments, H₁ comprises an amino acid sequence that has at least 90% sequence identity to SEQ ID NO: 69. In some embodiments, H₁ comprises an amino acid sequence that has at least 95% sequence identity to SEQ ID NO: 69. In some embodiments, H₁ comprises an amino acid sequence that has at least 99% sequence identity to SEQ ID NO: 69.

In some embodiments, H₁ comprises an amino acid sequence according to SEQ ID NO: 73. In some embodiments, H₁ comprises an amino acid sequence that has at least 80% sequence identity to SEQ ID NO: 73. In some embodiments, H₁ comprises an amino acid sequence that has at least 85% sequence identity to SEQ ID NO: 73. In some embodiments, H₁ comprises an amino acid sequence that has at least 90% sequence identity to SEQ ID NO: 73. In some embodiments, H₁ comprises an amino acid sequence that has at least 95% sequence identity to SEQ ID NO: 73. In some embodiments, H₁ comprises an amino acid sequence that has at least 99% sequence identity to SEQ ID NO: 73.

In some embodiments, H₁ₐ comprises an amino acid sequence according to SEQ ID NO: 69. In some embodiments, H₁ₐ comprises an amino acid sequence that has at least 80% sequence identity to SEQ ID NO: 69. In some embodiments, H₁ₐ comprises an amino acid sequence that has at least 85% sequence identity to SEQ ID NO: 69. In some embodiments, H₁ₐ comprises an amino acid sequence that has at least 90% sequence identity to SEQ ID NO: 69. In some embodiments, H₁ₐ comprises an amino acid sequence that has at least 95% sequence identity to SEQ ID NO: 69. In some embodiments, H₁ₐ comprises an amino acid sequence that has at least 99% sequence identity to SEQ ID NO: 69.

In some embodiments, H₁ₐ comprises an amino acid sequence according to SEQ ID NO: 73. In some embodiments, H₁ₐ comprises an amino acid sequence that has at least 80% sequence identity to SEQ ID NO: 73. In some embodiments, H₁ₐ comprises an amino acid sequence that has at least 85% sequence identity to SEQ ID NO: 73. In some embodiments, H₁ₐ comprises an amino acid sequence that has at least 90% sequence identity to SEQ ID NO: 73. In some embodiments, H₁ₐ comprises an amino acid sequence that has at least 95% sequence identity to SEQ ID NO: 73. In some embodiments, H₁ₐ comprises an amino acid sequence that has at least 99% sequence identity to SEQ ID NO: 73.

In some embodiments, H₁ or H₁ₐ or H₁ and H₁ₐ comprise a modified amino acid or non-natural amino acid, or a modified non-natural amino acid, or a combination thereof. In some embodiments, the modified amino acid or a modified non-natural amino acid comprises a post-translational modification. In some embodiments H₁ or H₁ₐ or H₁ and H₁ₐ comprise a modification including, but not limited to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to H₁ or H₁ₐ or H₁ and H₁ₐ including the peptide backbone, the amino acid side chains, and the terminus.

In some embodiments, H₁ comprises a linking moiety (L₃) that connects H₁ to P₁. In some embodiments, L₃ is a peptide sequence having at least 5 to no more than 50 amino acids. In some embodiments, L₃ is a peptide sequence having at least 10 to no more than 30 amino acids. In some embodiments, L₃ is a peptide sequence having at least 10 amino acids. In some embodiments, L₃ is a peptide sequence having at least 18 amino acids. In some embodiments, L₃ is a peptide sequence having at least 26 amino acids. In some embodiments, L₃ has a formula selected from the group consisting of (G₂S)ₙ, (GS)ₙ, (GSGGS)ₙ (SEQ ID NO: 62), (GGGS)ₙ (SEQ ID NO: 63), (GGGGS)ₙ (SEQ ID NO: 64), and (GSSGGS)ₙ (SEQ ID NO: 65), wherein n is an integer of at least 1. In some embodiments, L₃ comprises an amino acid sequence according to SEQ ID NO: 30.

In some embodiments, H₁ₐ comprises a linking moiety (L₁ₐ) that connects H₁ₐ to P₁ₐ. In some embodiments, L₁ₐ is a peptide sequence having at least 5 to no more than 50 amino acids. In some embodiments, L₁ₐ is a peptide sequence having at least 10 to no more than 30 amino acids. In some embodiments, L₁ₐ is a peptide sequence having at least 10 amino acids. In some embodiments, L₁ₐ is a peptide sequence having at least 18 amino acids. In some embodiments, L₁ₐ is a peptide sequence having at least 26 amino acids. In some embodiments, L₁ₐ has a formula selected from the group consisting of (G₂S)ₙ, (GS)ₙ, (GSGGS)ₙ (SEQ ID NO: 62), (GGGS)ₙ (SEQ ID NO: 63), (GGGGS)ₙ (SEQ ID NO: 64), and (GSSGGS)ₙ (SEQ ID NO: 65), wherein n is an integer of at least 1. In some embodiments, L₁ₐ comprises an amino acid sequence according to any one of SEQ ID NOs: 28-61.

Antibodies that Bind to TROP2 and CD3

In some embodiments, the isolated polypeptide or polypeptide complex comprises an amino acid sequence disclosed in Table 6 or a sequence substantially identical thereto (e.g., a sequence that has at least 90%, 95%, 96%, 97%, 98%, or 99% sequence identity). In some embodiments, the isolated polypeptide or polypeptide complex comprises an amino acid sequence with at least 95% sequence identity to SEQ ID NOs: 74-132, and 241-242. In some embodiments, the isolated polypeptide or polypeptide complex comprises an amino acid sequence with at least 95% sequence identity to SEQ ID NO: 82. In some embodiments, the isolated polypeptide or polypeptide complex comprises an amino acid sequence with at least 95% sequence identity to SEQ ID NO: 83.

TABLE 6
Polypeptide complex sequences

| | Amino Acid Sequence | |
|---|---|---|
| Construct Description | (N to C) | SEQ ID NO: |
| PC1: LC | DIQLTQSPSSLSASVGDRVSITCKASQDVSIAVA | 74 |
| TROP2 Fab LC | WYQQKPGKAPKLLIYSASYRYTGVPDRFSGSGS | |
| | GTDFTLTISSLQPEDFAVYYCQQHYITPLTFGAG | |
| | TKVEIKRTVAAPSVFIFPPSDEQLKSGTASVVCLL | |
| | NNFYPREAKVQWKVDNALQSGNSQESVTEQDS | |
| | KDSTYSLSSTLTLSKADYEKHKVYACEVTHQGL | |
| | SSPVTKSFNRGEC | |
| PC1: HC | QTVVTQEPSLTVSPGGTVTLTCRSSTGAVTTSNY | 75 |
| SP34.194 scFv (VH- | ANWVQQKPGQAPRGLIGGTNKRAPGTPARFSGS | |
| linker 1-VL) + Linker | LLGGKAALTLSGVQPEDEAEYYCALWYSNLWV | |
| 2 + TROP2 Fab HC | FGGGTKLTVLGGGGSGGGGSGGGGSEVQLVES | |
| | GGGLVQPGGSLKLSCAASGFTFNTYAMNWVRQ | |
| | APGKGLEWVARIRSKYNNYATYYADSVKDRFT | |
| | ISRDDSKNTAYLQMNNLKTEDTAVYYCVRHGN | |
| | FGNSYVSWFAYWGQGTLVTVSSGGGGSQVQLQ | |
| | QSGSELKKPGASVKVSCKASGYTFTNYGMNWV | |
| | KQAPGQGLKWMGWINTYTGEPTYTDDFKGRF | |
| | AFSLDTSVSTAYLQISSLKADDTAVYFCARGGF | |
| | GSSYWYFDVWGQGSLVTVSSASTKGPSVFPLAP | |
| | SSKSTSGGTAALGCLVKDYFPEPVTVSWNSGAL | |
| | TSGVHTFPAVLQSSGLYSLSSVVTVPSSSLGTQT | |
| | YICNVNHKPSNTKVDKKVEPKSC | |
| PC2: LC | QTVVTQEPSLTVSPGGTVTLTCRSSTGAVTTSNY | 76 |
| SP34.194 scFv (VH- | ANWVQQKPGQAPRGLIGGTNKRAPGTPARFSGS | |
| linker 1-VL) + Linker | LLGGKAALTLSGVQPEDEAEYYCALWYSNLWV | |
| 2 + TROP2 Fab LC | FGGGTKLTVLGGGGSGGGGSGGGGSEVQLVES | |
| | GGGLVQPGGSLKLSCAASGFTFNTYAMNWVRQ | |
| | APGKGLEWVARIRSKYNNYATYYADSVKDRFT | |
| | ISRDDSKNTAYLQMNNLKTEDTAVYYCVRHGN | |
| | FGNSYVSWFAYWGQGTLVTVSSGGGGSDIQLT | |
| | QSPSSLSASVGDRVSITCKASQDVSIAVAWYQQ | |
| | KPGKAPKLLIYSASYRYTGVPDRFSGSGSGTDFT | |
| | LTISSLQPEDFAVYYCQQHYITPLTFGAGTKVEI | |
| | KRTVAAPSVFIFPPSDEQLKSGTASVVCLLNNFY | |
| | PREAKVQWKVDNALQSGNSQESVTEQDSKDST | |
| | YSLSSTLTLSKADYEKHKVYACEVTHQGLSSPV | |
| | TKSFNRGEC | |
| PC2: HC | QVQLQQSGSELKKPGASVKVSCKASGYTFTNY | 77 |
| TROP2 Fab HC | GMNWVKQAPGQGLKWMGWINTYTGEPTYTD | |
| | DFKGRFAFSLDTSVSTAYLQISSLKADDTAVYFC | |
| | ARGGFGSSYWYFDVWGQGSLVTVSSASTKGPS | |
| | VFPLAPSSKSTSGGTAALGCLVKDYFPEPVTVSW | |
| | NSGALTSGVHTFPAVLQSSGLYSLSSVVTVPSSS | |
| | LGTQTYICNVNHKPSNTKVDKKVEPKSC | |
| PC3: LC | DIQLTQSPSSLSASVGDRVSITCKASQDVSIAVA | 78 |
| TROP2 Fab LC | WYQQKPGKAPKLLIYSASYRYTGVPDRFSGSGS | |
| | GTDFTLTISSLQPEDFAVYYCQQHYITPLTFGAG | |
| | TKVEIKRTVAAPSVFIFPPSDEQLKSGTASVVCLL | |
| | NNFYPREAKVQWKVDNALQSGNSQESVTEQDS | |
| | KDSTYSLSSTLTLSKADYEKHKVYACEVTHQGL | |
| | SSPVTKSFNRGEC | |
| PC3: HC | EVQLVESGGGLVQPGGSLKLSCAASGFTFNKYA | 79 |
| SP34.185 scFv (VH- | MNWVRQAPGKGLEWVARIRSKYNNYATYYAD | |
| linker 1-VL) + Linker | SVKDRFTISRDDSKNTAYLQMNNLKTEDTAVYY | |
| 2 + TROP2 Fab HC | CVRHGNFGNSYISYWAYWGQGTLVTVSSGGG | |
| | GSGGGGSGGGGSQTVVTQEPSLTVSPGGTVTLT | |
| | CGSSTGAVTSGNYPNWVQQKPGQAPRGLIGGT | |
| | KFLAPGTPARFSGSLLGGKAALTLSGVQPEDEAE | |
| | YYCVLWYSNRWVFGGGTKLTVLGGGGSQVQL | |
| | QQSGSELKKPGASVKVSCKASGYTFTNYGMNW | |
| | VKQAPGQGLKWMGWINTYTGEPTYTDDFKGR | |
| | FAFSLDTSVSTAYLQISSLKADDTAVYFCARGGF | |
| | GSSYWYFDVWGQGSLVTVSSASTKGPSVFPLAP | |
| | SSKSTSGGTAALGCLVKDYFPEPVTVSWNSGAL | |
| | TSGVHTFPAVLQSSGLYSLSSVVTVPSSSLGTQT | |
| | YICNVNHKPSNTKVDKKVEPKSC | |
| PC4: LC | EVQLVESGGGLVQPGGSLKLSCAASGFTFNKYA | 80 |
| SP34.185 scFv (VH- | MNWVRQAPGKGLEWVARIRSKYNNYATYYAD | |
| linker 1-VL) + Linker | SVKDRFTISRDDSKNTAYLQMNNLKTEDTAVYY | |
| 2 + TROP2 Fab LC | CVRHGNFGNSYISYWAYWGQGTLVTVSSGGG | |
| | GSGGGGSGGGGSQTVVTQEPSLTVSPGGTVTLT | |
| | CGSSTGAVTSGNYPNWVQQKPGQAPRGLIGGT | |
| | KFLAPGTPARFSGSLLGGKAALTLSGVQPEDEAE | |
| | YYCVLWYSNRWVFGGGTKLTVLGGGGSDIQLT | |
| | QSPSSLSASVGDRVSITCKASQDVSIAVAWYQQ | |
| | KPGKAPKLLIYSASYRYTGVPDRFSGSGSGTDFT | |
| | LTISSLQPEDFAVYYCQQHYITPLTFGAGTKVEI | |
| | KRTVAAPSVFIFPPSDEQLKSGTASVVCLLNNFY | |
| | PREAKVQWKVDNALQSGNSQESVTEQDSKDST | |
| | YSLSSTLTLSKADYEKHKVYACEVTHQGLSSPV | |
| | TKSFNRGEC | |
| PC4: HC | QVQLQQSGSELKKPGASVKVSCKASGYTFTNY | 81 |
| TROP2 Fab HC | GMNWVKQAPGQGLKWMGWINTYTGEPTYTD | |
| | DFKGRFAFSLDTSVSTAYLQISSLKADDTAVYFC | |
| | ARGGFGSSYWYFDVWGQGSLVTVSSASTKGPS | |
| | VFPLAPSSKSTSGGTAALGCLVKDYFPEPVTVSW | |
| | NSGALTSGVHTFPAVLQSSGLYSLSSVVTVPSSS | |
| | LGTQTYICNVNHKPSNTKVDKKVEPKSC | |
| PC5: LC | GGVDFCKIYSWPVCHQGGGGSGGLSGRSDAGSP | 82 |
| TROP2 Fab mask | LGLAGSGGSDIQLTQSPSSLSASVGDRVSITCKAS | |
| (SEQ ID NO: 24) + | QDVSIAVAWYQQKPGKAPKLLIYSASYRYTGVP | |
| cleavable linker 2 + | DRFSGSGSGTDFTLTISSLQPEDFAVYYCQQHYI | |
| TROP2 Fab LC | TPLTFGAGTKVEIKRTVAAPSVFIFPPSDEQLKSG | |
| | TASVVCLLNNFYPREAKVQWKVDNALQSGNSQ | |
| | ESVTEQDSKDSTYSLSSTLTLSKADYEKHKVYA | |
| | CEVTHQGLSSPVTKSFNRGEC | |
| PC5: HC | EVQLVESGGGLVQPGGSLRLSCAASGSTFYTAV | 83 |
| Anti-albumin (SEQ ID | MGWVRQAPGKGLEWVAAIRWTALTTSYADSV | |
| NO: 69) + Linker 3 + | KGRFTISRDGAKTTLYLQMNSLRPEDTAVYYCA | |
| SP34.185 scFv | ARGTLGLFTTADSYDYWGQGTLVTVSSGGGGS | |
| mask (SEQ ID NO: 27) + | GGGSGGVYCGPEFDESVGCMGGGGSGGGLSGR | |
| Cleavable Linker 1 + | SDAGSPLGLAGSGGGSEVQLVESGGGLVQPGGS | |
| SP34.185 scFv (V- | LKLSCAASGFTFNKYAMNWVRQAPGKGLEWV | |
| linker 1-VL) + Linker | ARIRSKYNNYATYYADSVKDRFTISRDDSKNTA | |
| 2 + TROP2 Fab HC | YLQMNNLKTEDTAVYYCVRHGNFGNSYISYW | |
| | AYWGQGTLVTVSSGGGGSGGGGSGGGGSQTVV | |
| | TQEPSLTVSPGGTVTLTCGSSTGAVTSGNYPNW | |
| | VQQKPGQAPRGLIGGTKFLAPGTPARFSGSLLG | |
| | GKAALTLSGVQPEDEAEYYCVLWYSNRWVFG | |
| | GGTKLTVLGGGGSQVQLQQSGSELKKPGASVK | |
| | VSCKASGYTFTNYGMNWVKQAPGQGLKWMG | |
| | WINTYTGEPTYTDDFKGRFAFSLDTSVSTAYLQI | |
| | SSLKADDTAVYFCARGGFGSSYWYFDVWGQG | |
| | SLVTVSSASTKGPSVFPLAPSSKSTSGGTAALGCL | |
| | VKDYFPEPVTVSWNSGALTSGVHTFPAVLQSSG | |
| | LYSLSSVVTVPSSSLGTQTYICNVNHKPSNTKVD | |
| | KKVEPKSC | |
| PC6: LC | GGSVLFCVKNLYCWVTGGGGSGGLSGRSDAGS | 84 |
| TROP2 Fab mask | PLGLAGSGGSDIQLTQSPSSLSASVGDRVSITCKA | |
| (SEQ ID NO: 23) + | SQDVSIAVAWYQQKPGKAPKLLIYSASYRYTGV | |
| cleavable linker 2 + | PDRFSGSGSGTDFTLTISSLQPEDFAVYYCQQHY | |
| TROP2 Fab LC | ITPLTFGAGTKVEIKRTVAAPSVFIFPPSDEQLKS | |
| | GTASVVCLLNNFYPREAKVQWKVDNALQSGNS | |
| | QESVTEQDSKDSTYSLSSTLTLSKADYEKHKVY | |
| | ACEVTHQGLSSPVTKSFNRGEC | |
| PC6: HC | EVQLVESGGGLVQPGGSLRLSCAASGSTFYTAV | 85 |
| Anti-albumin (SEQ ID | MGWVRQAPGKGLEWVAAIRWTALTTSYADSV | |
| NO: 69) + Linker 3 + | KGRFTISRDGAKTTLYLQMNSLRPEDTAVYYCA | |
| SP34.185 scFv | ARGTLGLFTTADSYDYWGQGTLVTVSSGGGGS | |
| mask (SEQ ID NO: 27) + | GGGSGGVYCGPEFDESVGCMGGGGSGGGLSGR | |
| Cleavable Linker 1 + | SDAGSPLGLAGSGGGSEVQLVESGGGLVQPGGS | |
| SP34.185 scFv (VH- | LKLSCAASGFTFNKYAMNWVRQAPGKGLEWV | |
| linker 1-VL) + Linker | ARIRSKYNNYATYYADSVKDRFTISRDDSKNTA | |
| 2 + TROP2 Fab HC | YLQMNNLKTEDTAVYYCVRHGNFGNSYISYW | |
| | AYWGQGTLVTVSSGGGGSGGGGSGGGGSQTVV | |
| | TQEPSLTVSPGGTVTLTCGSSTGAVTSGNYPNW | |
| | VQQKPGQAPRGLIGGTKFLAPGTPARFSGSLLG | |
| | GKAALTLSGVQPEDEAEYYCVLWYSNRWVFG | |
| | GGTKLTVLGGGGSQVQLQQSGSELKKPGASVK | |
| | VSCKASGYTFTNYGMNWVKQAPGQGLKWMG | |
| | WINTYTGEPTYTDDFKGRFAFSLDTSVSTAYLQI | |
| | SSLKADDTAVYFCARGGFGSSYWYFDVWGQG | |
| | SLVTVSSASTKGPSVFPLAPSSKSTSGGTAALGCL | |
| | VKDYFPEPVTVSWNSGALTSGVHTFPAVLQSSG | |
| | LYSLSSVVTVPSSSLGTQTYICNVNHKPSNTKVD | |
| | KKVEPKSC | |
| PC7: LC | EVQLVESGGGLVQPGGSLRLSCAASGSTFYTAV | 86 |
| Anti-albumin (SEQ ID | MGWVRQAPGKGLEWVAAIRWTALTTSYADSV | |
| NO: 69) + Linker 3 + | KGRFTISRDGAKTTLYLQMNSLRPEDTAVYYCA | |
| SP34.185 scFv | ARGTLGLFTTADSYDYWGQGTLVTVSSGGGGS | |
| mask (SEQ ID NO: 27) + | GGGSGGVYCGPEFDESVGCMGGGGSGGGLSGR | |
| Cleavable linker 1 + | SDAGSPLGLAGSGGGSEVQLVESGGGLVQPGGS | |
| SP34.185 scFv (VH- | LKLSCAASGFTFNKYAMNWVRQAPGKGLEWV | |
| linker 1-VL) + Linker | ARIRSKYNNYATYYADSVKDRFTISRDDSKNTA | |
| 2 + TROP2 Fab LC | YLQMNNLKTEDTAVYYCVRHGNFGNSYISYW | |
| | AYWGQGTLVTVSSGGGGSGGGGSGGGGSQTVV | |
| | TQEPSLTVSPGGTVTLTCGSSTGAVTSGNYPNW | |
| | VQQKPGQAPRGLIGGTKFLAPGTPARFSGSLLG | |
| | GKAALTLSGVQPEDEAEYYCVLWYSNRWVFG | |
| | GGTKLTVLGGGGSDIQLTQSPSSLSASVGDRVSI | |
| | TCKASQDVSIAVAWYQQKPGKAPKLLIYSASYR | |
| | YTGVPDRFSGSGSGTDFTLTISSLQPEDFAVYYC | |
| | QQHYITPLTFGAGTKVEIKRTVAAPSVFIFPPSD | |
| | EQLKSGTASVVCLLNNFYPREAKVQWKVDNAL | |
| | QSGNSQESVTEQDSKDSTYSLSSTLTLSKADYEK | |
| | HKVYACEVTHQGLSSPVTKSFNRGEC | |
| PC7: HC | GGVDFCKIYSWPVCHQGGGGSGGLSGRSDAGSP | 87 |
| TROP2 Fab mask | LGLAGSGGSQVQLQQSGSELKKPGASVKVSCKA | |
| (SEQ ID NO: 24) + | SGYTFTNYGMNWVKQAPGQGLKWMGWINTY | |
| cleavable linker 2 + | TGEPTYTDDFKGRFAFSLDTSVSTAYLQISSLKA | |
| TROP2 Fab HC | DDTAVYFCARGGFGSSYWYFDVWGQGSLVTV | |
| | SSASTKGPSVFPLAPSSKSTSGGTAALGCLVKDY | |
| | FPEPVTVSWNSGALTSGVHTFPAVLQSSGLYSLS | |
| | SVVTVPSSSLGTQTYICNVNHKPSNTKVDKKVEP | |
| | KSC | |
| PC8: LC | EVQLVESGGGLVQPGGSLRLSCAASGSTFYTAV | 88 |
| Anti-albumin (SEQ ID | MGWVRQAPGKGLEWVAAIRWTALTTSYADSV | |
| NO: 69) + Linker 3 + | KGRFTISRDGAKTTLYLQMNSLRPEDTAVYYCA | |
| SP34.185 scFv | ARGTLGLFTTADSYDYWGQGTLVTVSSGGGGS | |
| mask (SEQ ID NO: 27) + | GGGSGGVYCGPEFDESVGCMGGGGSGGGLSGR | |
| Cleavable linker 1 + | SDAGSPLGLAGSGGGSEVQLVESGGGLVQPGGS | |
| SP34.185 scFv (VH- | LKLSCAASGFTFNKYAMNWVRQAPGKGLEWV | |
| linker 1-VL) + Linker | ARIRSKYNNYATYYADSVKDRFTISRDDSKNTA | |
| 2 + TROP2 Fab LC | YLQMNNLKTEDTAVYYCVRHGNFGNSYISYW | |
| | AYWGQGTLVTVSSGGGGSGGGGSGGGGSQTVV | |
| | TQEPSLTVSPGGTVTLTCGSSTGAVTSGNYPNW | |
| | VQQKPGQAPRGLIGGTKFLAPGTPARFSGSLLG | |
| | GKAALTLSGVQPEDEAEYYCVLWYSNRWVFG | |
| | GGTKLTVLGGGGSDIQLTQSPSSLSASVGDRVSI | |
| | TCKASQDVSIAVAWYQQKPGKAPKLLIYSASYR | |
| | YTGVPDRFSGSGSGTDFTLTISSLQPEDFAVYYC | |
| | QQHYITPLTFGAGTKVEIKRTVAAPSVFIFPPSD | |
| | EQLKSGTASVVCLLNNFYPREAKVQWKVDNAL | |
| | QSGNSQESVTEQDSKDSTYSLSSTLTLSKADYEK | |
| | HKVYACEVTHQGLSSPVTKSFNRGEC | |
| PC8: HC | GGSVLFCVKNLYCWVTGGGGSGGLSGRSDAGS | 89 |
| TROP2 Fab mask | PLGLAGSGGSQVQLQQSGSELKKPGASVKVSCK | |
| (SEQ ID NO: 23) + | ASGYTFTNYGMNWVKQAPGQGLKWMGWINT | |
| cleavable linker 2 + | YTGEPTYTDDFKGRFAFSLDTSVSTAYLQISSLK | |
| TROP2 Fab HC | ADDTAVYFCARGGFGSSYWYFDVWGQGSLVT | |
| | VSSASTKGPSVFPLAPSSKSTSGGTAALGCLVKD | |
| | YFPEPVTVSWNSGALTSGVHTFPAVLQSSGLYSL | |
| | SSVVTVPSSSLGTQTYICNVNHKPSNTKVDKKVE | |
| | PKSC | |
| PC9: LC | GGVDFCKIYSWPVCHQGGGGSGGLSGRSDAGSP | 90 |
| TROP2 Fab mask | LGLAGSGGSDIQLTQSPSSLSASVGDRVSITCKAS | |
| (SEQ ID NO: 24) + | QDVSIAVAWYQQKPGKAPKLLIYSASYRYTGVP | |
| cleavable linker 2 + | DRFSGSGSGTDFTLTISSLQPEDFAVYYCQQHYI | |
| TROP2 Fab LC | TPLTFGAGTKVEIKRTVAAPSVFIFPPSDEQLKSG | |
| | TASVVCLLNNFYPREAKVQWKVDNALQSGNSQ | |
| | ESVTEQDSKDSTYSLSSTLTLSKADYEKHKVYA | |
| | CEVTHQGLSSPVTKSFNRGEC | |
| PC9: HC | EVQLVESGGGLVQPGGSLRLSCAASGSTFYTAV | 91 |
| Anti-albumin (SEQ ID | MGWVRQAPGKGLEWVAAIRWTALTTSYADSV | |
| NO: 69) + Linker 3 + | KGRFTISRDGAKTTLYLQMNSLRPEDTAVYYCA | |
| SP34.185 scFv | ARGTLGLFTTADSYDYWGQGTLVTVSSGGGGS | |
| mask (SEQ ID NO: 26) + | GGGSGGGSQCLGPEWEVCPYGGGGSGGGLSGR | |
| Cleavable linker 1 + | SDAGSPLGLAGSGGGSEVQLVESGGGLVQPGGS | |
| SP34.185 scFv (VH- | LKLSCAASGFTFNKYAMNWVRQAPGKGLEWV | |
| linker 1-VL) + Linker | ARIRSKYNNYATYYADSVKDRFTISRDDSKNTA | |
| 2 + TROP2 Fab HC | YLQMNNLKTEDTAVYYCVRHGNFGNSYISYW | |
| | AYWGQGTLVTVSSGGGGSGGGGSGGGGSQTVV | |
| | TQEPSLTVSPGGTVTLTCGSSTGAVTSGNYPNW | |
| | VQQKPGQAPRGLIGGTKFLAPGTPARFSGSLLG | |
| | GKAALTLSGVQPEDEAEYYCVLWYSNRWVFG | |
| | GGTKLTVLGGGGSQVQLQQSGSELKKPGASVK | |
| | VSCKASGYTFTNYGMNWVKQAPGQGLKWMG | |
| | WINTYTGEPTYTDDFKGRFAFSLDTSVSTAYLQI | |
| | SSLKADDTAVYFCARGGFGSSYWYFDVWGQG | |
| | SLVTVSSASTKGPSVFPLAPSSKSTSGGTAALGCL | |
| | VKDYFPEPVTVSWNSGALTSGVHTFPAVLQSSG | |
| | LYSLSSVVTVPSSSLGTQTYICNVNHKPSNTKVD | |
| | KKVEPKSC | |
| PC10: LC | EVQLVESGGGLVQPGGSLRLSCAASGSTFYTAV | 92 |
| Anti-albumin (SEQ ID | MGWVRQAPGKGLEWVAAIRWTALTTSYADSV | |
| NO: 69) + Linker 3 + | KGRFTISRDGAKTTLYLQMNSLRPEDTAVYYCA | |
| SP34.185 scFv | ARGTLGLFTTADSYDYWGQGTLVTVSSGGGGS | |
| mask (SEQ ID NO: 26) + | GGGSGGGSQCLGPEWEVCPYGGGGSGGGLSGR | |
| Cleavable linker 1 + | SDAGSPLGLAGSGGGSEVQLVESGGGLVQPGGS | |
| SP34.185 scFv (VH- | LKLSCAASGFTFNKYAMNWVRQAPGKGLEWV | |
| linker 1-VL) + Linker | ARIRSKYNNYATYYADSVKDRFTISRDDSKNTA | |
| 2 + TROP2 Fab LC | YLQMNNLKTEDTAVYYCVRHGNFGNSYISYW | |
| | AYWGQGTLVTVSSGGGGSGGGGSGGGGSQTVV | |
| | TQEPSLTVSPGGTVTLTCGSSTGAVTSGNYPNW | |
| | VQQKPGQAPRGLIGGTKFLAPGTPARFSGSLLG | |
| | GKAALTLSGVQPEDEAEYYCVLWYSNRWVFG | |
| | GGTKLTVLGGGGSDIQLTQSPSSLSASVGDRVSI | |
| | TCKASQDVSIAVAWYQQKPGKAPKLLIYSASYR | |
| | YTGVPDRFSGSGSGTDFTLTISSLQPEDFAVYYC | |
| | QQHYITPLTFGAGTKVEIKRTVAAPSVFIFPPSD | |
| | EQLKSGTASVVCLLNNFYPREAKVQWKVDNAL | |
| | QSGNSQESVTEQDSKDSTYSLSSTLTLSKADYEK | |
| | HKVYACEVTHQGLSSPVTKSFNRGEC | |
| PC10: HC | GGVDFCKIYSWPVCHQGGGGSGGLSGRSDAGSP | 93 |
| TROP2 Fab mask | LGLAGSGGSQVQLQQSGSELKKPGASVKVSCKA | |
| (SEQ ID NO: 24) + | SGYTFTNYGMNWVKQAPGQGLKWMGWINTY | |
| cleavable linker 2 + | TGEPTYTDDFKGRFAFSLDTSVSTAYLQISSLKA | |
| TROP2 Fab HC | DDTAVYFCARGGFGSSYWYFDVWGQGSLVTV | |
| | SSASTKGPSVFPLAPSSKSTSGGTAALGCLVKDY | |
| | FPEPVTVSWNSGALTSGVHTFPAVLQSSGLYSLS | |
| | SVVTVPSSSLGTQTYICNVNHKPSNTKVDKKVEP | |
| | KSC | |
| PC11: LC | GGSVLFCVKNLYCWVTGGGGSGGLSGRSDAGS | 94 |
| TROP2 Fab mask | PLGLAGSGGSDIQLTQSPSSLSASVGDRVSITCKA | |
| (SEQ ID NO: 23) + | SQDVSIAVAWYQQKPGKAPKLLIYSASYRYTGV | |
| Cleavable linker 2 + | PDRFSGSGSGTDFTLTISSLQPEDFAVYYCQQHY | |
| TROP2 Fab LC | ITPLTFGAGTKVEIKRTVAAPSVFIFPPSDEQLKS | |
| | GTASVVCLLNNFYPREAKVQWKVDNALQSGNS | |
| | QESVTEQDSKDSTYSLSSTLTLSKADYEKHKVY | |
| | ACEVTHQGLSSPVTKSFNRGEC | |
| PC11: HC | EVQLVESGGGLVQPGGSLRLSCAASGSTFYTAV | 95 |
| Anti-albumin (SEQ ID | MGWVRQAPGKGLEWVAAIRWTALTTSYADSV | |
| NO: 69) + Linker 3 + | KGRFTISRDGAKTTLYLQMNSLRPEDTAVYYCA | |
| SP34.185 scFv | ARGTLGLFTTADSYDYWGQGTLVTVSSGGGGS | |
| mask (SEQ ID NO: 26) + | GGGSGGGSQCLGPEWEVCPYGGGGSGGGLSGR | |
| Cleavable linker 1 + | SDAGSPLGLAGSGGGSEVQLVESGGGLVQPGGS | |
| SP34.185 scFv (VH- | LKLSCAASGFTFNKYAMNWVRQAPGKGLEWV | |
| linker 1-VL) + Linker | ARIRSKYNNYATYYADSVKDRFTISRDDSKNTA | |
| 2 + TROP2 Fab HC | YLQMNNLKTEDTAVYYCVRHGNFGNSYISYW | |
| | AYWGQGTLVTVSSGGGGSGGGGSGGGGSQTVV | |
| | TQEPSLTVSPGGTVTLTCGSSTGAVTSGNYPNW | |
| | VQQKPGQAPRGLIGGTKFLAPGTPARFSGSLLG | |
| | GKAALTLSGVQPEDEAEYYCVLWYSNRWVFG | |
| | GGTKLTVLGGGGSQVQLQQSGSELKKPGASVK | |
| | VSCKASGYTFTNYGMNWVKQAPGQGLKWMG | |
| | WINTYTGEPTYTDDFKGRFAFSLDTSVSTAYLQI | |
| | SSLKADDTAVYFCARGGFGSSYWYFDVWGQG | |
| | SLVTVSSASTKGPSVFPLAPSSKSTSGGTAALGCL | |
| | VKDYFPEPVTVSWNSGALTSGVHTFPAVLQSSG | |
| | LYSLSSVVTVPSSSLGTQTYICNVNHKPSNTKVD | |
| | KKVEPKSC | |
| PC12: LC | EVQLVESGGGLVQPGGSLRLSCAASGSTFYTAV | 96 |
| Anti-albumin (SEQ ID | MGWVRQAPGKGLEWVAAIRWTALTTSYADSV | |
| NO: 69) + Linker 3 + | KGRFTISRDGAKTTLYLQMNSLRPEDTAVYYCA | |
| SP34.185 scFv | ARGTLGLFTTADSYDYWGQGTLVTVSSGGGGS | |
| mask (SEQ ID NO: 26) + | GGGSGGGSQCLGPEWEVCPYGGGGSGGGLSGR | |
| Cleavable linker 1 + | SDAGSPLGLAGSGGGSEVQLVESGGGLVQPGGS | |
| SP34.185 scFv (VH- | LKLSCAASGFTFNKYAMNWVRQAPGKGLEWV | |
| linker 1-VL) + Linker | ARIRSKYNNYATYYADSVKDRFTISRDDSKNTA | |
| 2 + TROP2 Fab LC | YLQMNNLKTEDTAVYYCVRHGNFGNSYISYW | |
| | AYWGQGTLVTVSSGGGGSGGGGSGGGGSQTVV | |
| | TQEPSLTVSPGGTVTLTCGSSTGAVTSGNYPNW | |
| | VQQKPGQAPRGLIGGTKFLAPGTPARFSGSLLG | |
| | GKAALTLSGVQPEDEAEYYCVLWYSNRWVFG | |
| | GGTKLTVLGGGGSDIQLTQSPSSLSASVGDRVSI | |
| | TCKASQDVSIAVAWYQQKPGKAPKLLIYSASYR | |
| | YTGVPDRFSGSGSGTDFTLTISSLQPEDFAVYYC | |
| | QQHYITPLTFGAGTKVEIKRTVAAPSVFIFPPSD | |
| | EQLKSGTASVVCLLNNFYPREAKVQWKVDNAL | |
| | QSGNSQESVTEQDSKDSTYSLSSTLTLSKADYEK | |
| | HKVYACEVTHQGLSSPVTKSFNRGEC | |
| PC12: HC | GGSVLFCVKNLYCWVTGGGGSGGLSGRSDAGS | 97 |
| TROP2 Fab mask | PLGLAGSGGSQVQLQQSGSELKKPGASVKVSCK | |
| (SEQ ID NO: 23) + | ASGYTFTNYGMNWVKQAPGQGLKWMGWINT | |
| cleavable linker 2 + | YTGEPTYTDDFKGRFAFSLDTSVSTAYLQISSLK | |
| TROP2 Fab HC | ADDTAVYFCARGGFGSSYWYFDVWGQGSLVT | |
| | VSSASTKGPSVFPLAPSSKSTSGGTAALGCLVKD | |
| | YFPEPVTVSWNSGALTSGVHTFPAVLQSSGLYSL | |
| | SSVVTVPSSSLGTQTYICNVNHKPSNTKVDKKVE | |
| | PKSC | |
| PC13: LC | GGVDFCKIYSWPVCHQGGGGSGGLSGRSDAGSP | 98 |
| TROP2 Fab mask | LGLAGSGGSDIQLTQSPSSLSASVGDRVSITCKAS | |
| (SEQ ID NO: 24) + | QDVSIAVAWYQQKPGKAPKLLIYSASYRYTGVP | |
| cleavable linker 2 + | DRFSGSGSGTDFTLTISSLQPEDFAVYYCQQHYI | |
| TROP2 Fab LC | TPLTFGAGTKVEIKRTVAAPSVFIFPPSDEQLKSG | |
| | TASVVCLLNNFYPREAKVQWKVDNALQSGNSQ | |
| | ESVTEQDSKDSTYSLSSTLTLSKADYEKHKVYA | |
| | CEVTHQGLSSPVTKSFNRGEC | |
| PC13: HC | EVQLVESGGGLVQPGNSLRLSCAASGFTFSKFG | 99 |
| 10G + Linker 3 + | MSWVRQAPGKGLEWVSSISGSGRDTLYADSVK | |
| SP34.185 scFv | GRFTISRDNAKTTLYLQMNSLRPEDTAVYYCTI | |
| mask (SEQ ID NO: 27) + | GGSLSVSSQGTLVTVSSGGGGSGGGSGGVYCGP | |
| Cleavable linker 1 + | EFDESVGCMGGGGSGGGLSGRSDAGSPLGLAGS | |
| SP34.185 scFv (VH- | GGGSEVQLVESGGGLVQPGGSLKLSCAASGFTF | |
| linker 1-VL) + Linker | NKYAMNWVRQAPGKGLEWVARIRSKYNNYAT | |
| 2 + TROP2 Fab HC | YYADSVKDRFTISRDDSKNTAYLQMNNLKTEDT | |
| | AVYYCVRHGNFGNSYISYWAYWGQGTLVTVS | |
| | SGGGGSGGGGSGGGGSQTVVTQEPSLTVSPGGT | |
| | VTLTCGSSTGAVTSGNYPNWVQQKPGQAPRGLI | |
| | GGTKFLAPGTPARFSGSLLGGKAALTLSGVQPE | |
| | DEAEYYCVLWYSNRWVFGGGTKLTVLGGGGS | |
| | QVQLQQSGSELKKPGASVKVSCKASGYTFTNY | |
| | GMNWVKQAPGQGLKWMGWINTYTGEPTYTD | |
| | DFKGRFAFSLDTSVSTAYLQISSLKADDTAVYFC | |
| | ARGGFGSSYWYFDVWGQGSLVTVSSASTKGPS | |
| | VFPLAPSSKSTSGGTAALGCLVKDYFPEPVTVSW | |
| | NSGALTSGVHTFPAVLQSSGLYSLSSVVTVPSSS | |
| | LGTQTYICNVNHKPSNTKVDKKVEPKSC | |
| PC14: LC | GGSVLFCVKNLYCWVTGGGGSGGLSGRSDAGS | 100 |
| TROP2 Fab mask | PLGLAGSGGSDIQLTQSPSSLSASVGDRVSITCKA | |
| (SEQ ID NO: 23) + | SQDVSIAVAWYQQKPGKAPKLLIYSASYRYTGV | |
| cleavable linker 2 + | PDRFSGSGSGTDFTLTISSLQPEDFAVYYCQQHY | |
| TROP2 Fab LC | ITPLTFGAGTKVEIKRTVAAPSVFIFPPSDEQLKS | |
| | GTASVVCLLNNFYPREAKVQWKVDNALQSGNS | |
| | QESVTEQDSKDSTYSLSSTLTLSKADYEKHKVY | |
| | ACEVTHQGLSSPVTKSFNRGEC | |
| PC14: HC | EVQLVESGGGLVQPGNSLRLSCAASGFTFSKFG | 101 |
| 10G + Linker 3 + | MSWVRQAPGKGLEWVSSISGSGRDTLYADSVK | |
| SP34.185 scFv | GRFTISRDNAKTTLYLQMNSLRPEDTAVYYCTI | |
| mask (SEQ ID NO: 27) + | GGSLSVSSQGTLVTVSSGGGGSGGGSGGVYCGP | |
| Cleavable linker 1 + | EFDESVGCMGGGGSGGGLSGRSDAGSPLGLAGS | |
| SP34.185 scFv (VH - | GGGSEVQLVESGGGLVQPGGSLKLSCAASGFTF | |
| linker 1 - VL) + Linker | NKYAMNWVRQAPGKGLEWVARIRSKYNNYAT | |
| 2 + TROP2 Fab HC | YYADSVKDRFTISRDDSKNTAYLQMNNLKTEDT | |
| | AVYYCVRHGNFGNSYISYWAYWGQGTLVTVS | |
| | SGGGGSGGGGSGGGGSQTVVTQEPSLTVSPGGT | |
| | VTLTCGSSTGAVTSGNYPNWVQQKPGQAPRGLI | |
| | GGTKFLAPGTPARFSGSLLGGKAALTLSGVQPE | |
| | DEAEYYCVLWYSNRWVFGGGTKLTVLGGGGS | |
| | QVQLQQSGSELKKPGASVKVSCKASGYTFTNY | |
| | GMNWVKQAPGQGLKWMGWINTYTGEPTYTD | |
| | DFKGRFAFSLDTSVSTAYLQISSLKADDTAVYFC | |
| | ARGGFGSSYWYFDVWGQGSLVTVSSASTKGPS | |
| | VFPLAPSSKSTSGGTAALGCLVKDYFPEPVTVSW | |
| | NSGALTSGVHTFPAVLQSSGLYSLSSVVTVPSSS | |
| | LGTQTYICNVNHKPSNTKVDKKVEPKSC | |
| PC15: LC | EVQLVESGGGLVQPGNSLRLSCAASGFTFSKFG | 102 |
| 10G + Linker 3 + | MSWVRQAPGKGLEWVSSISGSGRDTLYADSVK | |
| SP34.185 scFv | GRFTISRDNAKTTLYLQMNSLRPEDTAVYYCTI | |
| mask (SEQ ID NO: 27) + | GGSLSVSSQGTLVTVSSGGGGSGGGSGGVYCGP | |
| Cleavable linker 1 + | EFDESVGCMGGGGSGGGLSGRSDAGSPLGLAGS | |
| SP34.185 scFv (VH- | GGGSEVQLVESGGGLVQPGGSLKLSCAASGFTF | |
| linker 1-VL) + Linker | NKYAMNWVRQAPGKGLEWVARIRSKYNNYAT | |
| 2 + TROP2 Fab LC | YYADSVKDRFTISRDDSKNTAYLQMNNLKTEDT | |
| | AVYYCVRHGNFGNSYISYWAYWGQGTLVTVS | |
| | SGGGGSGGGGSGGGGSQTVVTQEPSLTVSPGGT | |
| | VTLTCGSSTGAVTSGNYPNWVQQKPGQAPRGLI | |
| | GGTKFLAPGTPARFSGSLLGGKAALTLSGVQPE | |
| | DEAEYYCVLWYSNRWVFGGGTKLTVLGGGGS | |
| | DIQLTQSPSSLSASVGDRVSITCKASQDVSIAVA | |
| | WYQQKPGKAPKLLIYSASYRYTGVPDRFSGSGS | |
| | GTDFTLTISSLQPEDFAVYYCQQHYITPLTFGAG | |
| | TKVEIKRTVAAPSVFIFPPSDEQLKSGTASVVCLL | |
| | NNFYPREAKVQWKVDNALQSGNSQESVTEQDS | |
| | KDSTYSLSSTLTLSKADYEKHKVYACEVTHQGL | |
| | SSPVTKSFNRGEC | |
| PC15: HC | GGVDFCKIYSWPVCHQGGGGSGGLSGRSDAGSP | 103 |
| TROP2 Fab mask | LGLAGSGGSQVQLQQSGSELKKPGASVKVSCKA | |
| (SEQ ID NO: 24) + | SGYTFTNYGMNWVKQAPGQGLKWMGWINTY | |
| cleavable linker 2 + | TGEPTYTDDFKGRFAFSLDTSVSTAYLQISSLKA | |
| TROP2 Fab HC | DDTAVYFCARGGFGSSYWYFDVWGQGSLVTV | |
| | SSASTKGPSVFPLAPSSKSTSGGTAALGCLVKDY | |
| | FPEPVTVSWNSGALTSGVHTFPAVLQSSGLYSLS | |
| | SVVTVPSSSLGTQTYICNVNHKPSNTKVDKKVEP | |
| | KSC | |
| PC16: LC | EVQLVESGGGLVQPGNSLRLSCAASGFTFSKFG | 104 |
| 10G + Linker 3 + | MSWVRQAPGKGLEWVSSISGSGRDTLYADSVK | |
| SP34.185 scFv | GRFTISRDNAKTTLYLQMNSLRPEDTAVYYCTI | |
| mask (SEQ ID NO: 27) + | GGSLSVSSQGTLVTVSSGGGGSGGGSGGVYCGP | |
| Cleavable linker 1 + | EFDESVGCMGGGGSGGGLSGRSDAGSPLGLAGS | |
| SP34.185 scFv (VH- | GGGSEVQLVESGGGLVQPGGSLKLSCAASGFTF | |
| linker 1-VL) + Linker | NKYAMNWVRQAPGKGLEWVARIRSKYNNYAT | |
| 2 + TROP2 Fab LC | YYADSVKDRFTISRDDSKNTAYLQMNNLKTEDT | |
| | AVYYCVRHGNFGNSYISYWAYWGQGTLVTVS | |
| | SGGGGSGGGGSGGGGSQTVVTQEPSLTVSPGGT | |
| | VTLTCGSSTGAVTSGNYPNWVQQKPGQAPRGLI | |
| | GGTKFLAPGTPARFSGSLLGGKAALTLSGVQPE | |
| | DEAEYYCVLWYSNRWVFGGGTKLTVLGGGGS | |
| | DIQLTQSPSSLSASVGDRVSITCKASQDVSIAVA | |
| | WYQQKPGKAPKLLIYSASYRYTGVPDRFSGSGS | |
| | GTDFTLTISSLQPEDFAVYYCQQHYITPLTFGAG | |
| | TKVEIKRTVAAPSVFIFPPSDEQLKSGTASVVCLL | |
| | NNFYPREAKVQWKVDNALQSGNSQESVTEQDS | |
| | KDSTYSLSSTLTLSKADYEKHKVYACEVTHQGL | |
| | SSPVTKSFNRGEC | |
| PC16: HC | GGSVLFCVKNLYCWVTGGGGSGGLSGRSDAGS | 105 |
| TROP2 Fab mask | PLGLAGSGGSQVQLQQSGSELKKPGASVKVSCK | |
| (SEQ ID NO: 23) + | ASGYTFTNYGMNWVKQAPGQGLKWMGWINT | |
| cleavable linker 2 + | YTGEPTYTDDFKGRFAFSLDTSVSTAYLQISSLK | |
| TROP2 Fab HC | ADDTAVYFCARGGFGSSYWYFDVWGQGSLVT | |
| | VSSASTKGPSVFPLAPSSKSTSGGTAALGCLVKD | |
| | YFPEPVTVSWNSGALTSGVHTFPAVLQSSGLYSL | |
| | SSVVTVPSSSLGTQTYICNVNHKPSNTKVDKKVE | |
| | PKSC | |
| PC17: LC | GGIDFCMLYNWPICAGGGGGSSGGSAAGLLAPP | 106 |
| | GGLSGRSDAGGGGSDIQLTQSPSSLSASVGDRVS | |
| | ITCKASQDVSIAVAWYQQKPGKAPKLLIYSASY | |
| | RYTGVPDRFSGSGSGTDFTLTISSLQPEDFAVYY | |
| | CQQHYITPLTFGAGTKVEIKRTVAAPSVFIFPPS | |
| | DEQLKSGTASVVCLLNNFYPREAKVQWKVDNA | |
| | LQSGNSQESVTEQDSKDSTYSLSSTLTLSKADYE | |
| | KHKVYACEVTHQGLSSPVTKSFNRGEC | |
| PC17: HC | EVQLVESGGGLVQPGNSLRLSCAASGFTFSKFG | 107 |
| | MSWVRQAPGKGLEWVSSISGSGRDTLYADSVK | |
| | GRFTISRDNAKTTLYLQMNSLRPEDTAVYYCTI | |
| | GGSLSVSSQGTLVTVSSGGGGSGGGSGGVYCGP | |
| | EFDESVGCMGSSGGSAAGLLAPPGGLSGRSDAG | |
| | GGGSEVQLVESGGGLVQPGGSLKLSCAASGFTF | |
| | NKYAMNWVRQAPGKGLEWVARIRSKYNNYAT | |
| | YYADSVKDRFTISRDDSKNTAYLQMNNLKTEDT | |
| | AVYYCVRHGNFGNSYISYWAYWGQGTLVTVS | |
| | SGGGGSGGGGSGGGGSQTVVTQEPSLTVSPGGT | |
| | VTLTCGSSTGAVTSGNYPNWVQQKPGQAPRGLI | |
| | GGTKFLAPGTPARFSGSLLGGKAALTLSGVQPE | |
| | DEAEYYCVLWYSNRWVFGGGTKLTVLGGGGS | |
| | QVQLQQSGSELKKPGASVKVSCKASGYTFTNY | |
| | GMNWVKQAPGQGLKWMGWINTYTGEPTYTD | |
| | DFKGRFAFSLDTSVSTAYLQISSLKADDTAVYFC | |
| | ARGGFGSSYWYFDVWGQGSLVTVSSASTKGPS | |
| | VFPLAPSSKSTSGGTAALGCLVKDYFPEPVTVSW | |
| | NSGALTSGVHTFPAVLQSSGLYSLSSVVTVPSSS | |
| | LGTQTYICNVNHKPSNTKVDKKVEPKSC | |
| PC18: LC | GGIDFCMLYNWPICAGGGGGSSGGSAAGLLAPP | 108 |
| | GGLSGRSDAGGGGSDIQLTQSPSSLSASVGDRVS | |
| | ITCKASQDVSIAVAWYQQKPGKAPKLLIYSASY | |
| | RYTGVPDRFSGSGSGTDFTLTISSLQPEDFAVYY | |
| | CQQHYITPLTFGAGTKVEIKRTVAAPSVFIFPPS | |
| | DEQLKSGTASVVCLLNNFYPREAKVQWKVDNA | |
| | LQSGNSQESVTEQDSKDSTYSLSSTLTLSKADYE | |
| | KHKVYACEVTHQGLSSPVTKSFNRGEC | |
| PC18: HC | EVQLVESGGGLVQPGGSLRLSCAASGSTFYTAV | 109 |
| | MGWVRQAPGKGLEWVAAIRWTALTTSYADSV | |
| | KGRFTISRDGAKTTLYLQMNSLRPEDTAVYYCA | |
| | ARGTLGLFTTADSYDYWGQGTLVTVSSGGGGS | |
| | GGGSGGVYCGPEFDESVGCMGSSGGSAAGLLAP | |
| | PGGLSGRSDAGGGGSEVQLVESGGGLVQPGGSL | |
| | KLSCAASGFTFNKYAMNWVRQAPGKGLEWVA | |
| | RIRSKYNNYATYYADSVKDRFTISRDDSKNTAY | |
| | LQMNNLKTEDTAVYYCVRHGNFGNSYISYWA | |
| | YWGQGTLVTVSSGGGGSGGGGSGGGGSQTVVT | |
| | QEPSLTVSPGGTVTLTCGSSTGAVTSGNYPNWV | |
| | QQKPGQAPRGLIGGTKFLAPGTPARFSGSLLGG | |
| | KAALTLSGVQPEDEAEYYCVLWYSNRWVFGG | |
| | GTKLTVLGGGGSQVQLQQSGSELKKPGASVKVS | |
| | CKASGYTFTNYGMNWVKQAPGQGLKWMGWI | |
| | NTYTGEPTYTDDFKGRFAFSLDTSVSTAYLQISS | |
| | LKADDTAVYFCARGGFGSSYWYFDVWGQGSL | |
| | VTVSSASTKGPSVFPLAPSSKSTSGGTAALGCLV | |
| | KDYFPEPVTVSWNSGALTSGVHTFPAVLQSSGL | |
| | YSLSSVVTVPSSSLGTQTYICNVNHKPSNTKVDK | |
| | KVEPKSC | |
| PC19: LC | EVQLVESGGGLVQPGGSLRLSCAASGSTFYTAV | 110 |
| | MGWVRQAPGKGLEWVAAIRWTALTTSYADSV | |
| | KGRFTISRDGAKTTLYLQMNSLRPEDTAVYYCA | |
| | ARGTLGLFTTADSYDYWGQGTLVTVSSGGGGS | |
| | GGGSGGVYCGPEFDESVGCMGSSGGSAAGLLAP | |
| | PGGLSGRSDAGGGGSEVQLVESGGGLVQPGGSL | |
| | KLSCAASGFTFNKYAMNWVRQAPGKGLEWVA | |
| | RIRSKYNNYATYYADSVKDRFTISRDDSKNTAY | |
| | LQMNNLKTEDTAVYYCVRHGNFGNSYISYWA | |
| | YWGQGTLVTVSSGGGGSGGGGSGGGGSQTVVT | |
| | QEPSLTVSPGGTVTLTCGSSTGAVTSGNYPNWV | |
| | QQKPGQAPRGLIGGTKFLAPGTPARFSGSLLGG | |
| | KAALTLSGVQPEDEAEYYCVLWYSNRWVFGG | |
| | GTKLTVLGGGGSDIQLTQSPSSLSASVGDRVSITC | |
| | KASQDVSIAVAWYQQKPGKAPKLLIYSASYRYT | |
| | GVPDRFSGSGSGTDFTLTISSLQPEDFAVYYCQQ | |
| | HYITPLTFGAGTKVEIKRTVAAPSVFIFPPSDEQL | |
| | KSGTASVVCLLNNFYPREAKVQWKVDNALQSG | |
| | NSQESVTEQDSKDSTYSLSSTLTLSKADYEKHKV | |
| | YACEVTHQGLSSPVTKSFNRGEC | |
| PC19: HC | GGIDFCMLYNWPICAGGGGGSSGGSAAGLLAPP | 111 |
| | GGLSGRSDAGGGGSQVQLQQSGSELKKPGASVK | |
| | VSCKASGYTFTNYGMNWVKQAPGQGLKWMG | |
| | WINTYTGEPTYTDDFKGRFAFSLDTSVSTAYLQI | |
| | SSLKADDTAVYFCARGGFGSSYWYFDVWGQG | |
| | SLVTVSSASTKGPSVFPLAPSSKSTSGGTAALGCL | |
| | VKDYFPEPVTVSWNSGALTSGVHTFPAVLQSSG | |
| | LYSLSSVVTVPSSSLGTQTYICNVNHKPSNTKVD | |
| | KKVEPKSC | |
| PC20: LC | EVQLVESGGGLVQPGNSLRLSCAASGFTFSKFG | 112 |
| | MSWVRQAPGKGLEWVSSISGSGRDTLYADSVK | |
| | GRFTISRDNAKTTLYLQMNSLRPEDTAVYYCTI | |
| | GGSLSVSSQGTLVTVSSGGGGSGGGSGGVYCGP | |
| | EFDESVGCMGSSGGSAAGLLAPPGGLSGRSDAG | |
| | GGGSEVQLVESGGGLVQPGGSLKLSCAASGFTF | |
| | NKYAMNWVRQAPGKGLEWVARIRSKYNNYAT | |
| | YYADSVKDRFTISRDDSKNTAYLQMNNLKTEDT | |
| | AVYYCVRHGNFGNSYISYWAYWGQGTLVTVS | |
| | SGGGGSGGGGSGGGGSQTVVTQEPSLTVSPGGT | |
| | VTLTCGSSTGAVTSGNYPNWVQQKPGQAPRGLI | |
| | GGTKFLAPGTPARFSGSLLGGKAALTLSGVQPE | |
| | DEAEYYCVLWYSNRWVFGGGTKLTVLGGGGS | |
| | DIQLTQSPSSLSASVGDRVSITCKASQDVSIAVA | |
| | WYQQKPGKAPKLLIYSASYRYTGVPDRFSGSGS | |
| | GTDFTLTISSLQPEDFAVYYCQQHYITPLTFGAG | |
| | TKVEIKRTVAAPSVFIFPPSDEQLKSGTASVVCLL | |
| | NNFYPREAKVQWKVDNALQSGNSQESVTEQDS | |
| | KDSTYSLSSTLTLSKADYEKHKVYACEVTHQGL | |
| | SSPVTKSFNRGEC | |
| PC20: HC | GGIDFCMLYNWPICAGGGGGSSGGSAAGLLAPP | 113 |
| | GGLSGRSDAGGGGSQVQLQQSGSELKKPGASVK | |
| | VSCKASGYTFTNYGMNWVKQAPGQGLKWMG | |
| | WINTYTGEPTYTDDFKGRFAFSLDTSVSTAYLQI | |
| | SSLKADDTAVYFCARGGFGSSYWYFDVWGQG | |
| | SLVTVSSASTKGPSVFPLAPSSKSTSGGTAALGCL | |
| | VKDYFPEPVTVSWNSGALTSGVHTFPAVLQSSG | |
| | LYSLSSVVTVPSSSLGTQTYICNVNHKPSNTKVD | |
| | KKVEPKSC | |
| PC21: LC | GGVDFCKIYSWPVCHQGGGGSSGGSAAGLLAPP | 114 |
| | GGLSGRSDAGGGGSDIQLTQSPSSLSASVGDRVS | |
| | ITCKASQDVSIAVAWYQQKPGKAPKLLIYSASY | |
| | RYTGVPDRFSGSGSGTDFTLTISSLQPEDFAVYY | |
| | CQQHYITPLTFGAGTKVEIKRTVAAPSVFIFPPS | |
| | DEQLKSGTASVVCLLNNFYPREAKVQWKVDNA | |
| | LQSGNSQESVTEQDSKDSTYSLSSTLTLSKADYE | |
| | KHKVYACEVTHQGLSSPVTKSFNRGEC | |
| PC21: HC | EVQLVESGGGLVQPGGSLRLSCAASGSTFYTAV | 115 |
| | MGWVRQAPGKGLEWVAAIRWTALTTSYADSV | |
| | KGRFTISRDGAKTTLYLQMNSLRPEDTAVYYCA | |
| | ARGTLGLFTTADSYDYWGQGTLVTVSSGGGGS | |
| | GGGSGGVYCGPEFDESVGCMGSSGGSAAGLLAP | |
| | PGGLSGRSDAGGGGSEVQLVESGGGLVQPGGSL | |
| | KLSCAASGFTFNKYAMNWVRQAPGKGLEWVA | |
| | RIRSKYNNYATYYADSVKDRFTISRDDSKNTAY | |
| | LQMNNLKTEDTAVYYCVRHGNFGNSYISYWA | |
| | YWGQGTLVTVSSGGGGSGGGGSGGGGSQTVVT | |
| | QEPSLTVSPGGTVTLTCGSSTGAVTSGNYPNWV | |
| | QQKPGQAPRGLIGGTKFLAPGTPARFSGSLLGG | |
| | KAALTLSGVQPEDEAEYYCVLWYSNRWVFGG | |
| | GTKLTVLGGGGSQVQLQQSGSELKKPGASVKVS | |
| | CKASGYTFTNYGMNWVKQAPGQGLKWMGWI | |
| | NTYTGEPTYTDDFKGRFAFSLDTSVSTAYLQISS | |
| | LKADDTAVYFCARGGFGSSYWYFDVWGQGSL | |
| | VTVSSASTKGPSVFPLAPSSKSTSGGTAALGCLV | |
| | KDYFPEPVTVSWNSGALTSGVHTFPAVLQSSGL | |
| | YSLSSVVTVPSSSLGTQTYICNVNHKPSNTKVDK | |
| | KVEPKSC | |
| PC22: LC | GGIDFCMLYNWPICAGGGGGSGGLSGRSDAGSP | 116 |
| | LGLAGSGGSDIQLTQSPSSLSASVGDRVSITCKAS | |
| | QDVSIAVAWYQQKPGKAPKLLIYSASYRYTGVP | |
| | DRFSGSGSGTDFTLTISSLQPEDFAVYYCQQHYI | |
| | TPLTFGAGTKVEIKRTVAAPSVFIFPPSDEQLKSG | |
| | TASVVCLLNNFYPREAKVQWKVDNALQSGNSQ | |
| | ESVTEQDSKDSTYSLSSTLTLSKADYEKHKVYA | |
| | CEVTHQGLSSPVTKSFNRGEC | |
| PC22: HC | EVQLVESGGGLVQPGGSLRLSCAASGSTFYTAV | 117 |
| | MGWVRQAPGKGLEWVAAIRWTALTTSYADSV | |
| | KGRFTISRDGAKTTLYLQMNSLRPEDTAVYYCA | |
| | ARGTLGLFTTADSYDYWGQGTLVTVSSGGGGS | |
| | GGGSGGVYCGPEFDESVGCMGGGGSGGGLSGR | |
| | SDAGSPLGLAGSGGGSEVQLVESGGGLVQPGGS | |
| | LKLSCAASGFTFNKYAMNWVRQAPGKGLEWV | |
| | ARIRSKYNNYATYYADSVKDRFTISRDDSKNTA | |
| | YLQMNNLKTEDTAVYYCVRHGNFGNSYISYW | |
| | AYWGQGTLVTVSSGGGGSGGGGSGGGGSQTVV | |
| | TQEPSLTVSPGGTVTLTCGSSTGAVTSGNYPNW | |
| | VQQKPGQAPRGLIGGTKFLAPGTPARFSGSLLG | |
| | GKAALTLSGVQPEDEAEYYCVLWYSNRWVFG | |
| | GGTKLTVLGGGGSQVQLQQSGSELKKPGASVK | |
| | VSCKASGYTFTNYGMNWVKQAPGQGLKWMG | |
| | WINTYTGEPTYTDDFKGRFAFSLDTSVSTAYLQI | |
| | SSLKADDTAVYFCARGGFGSSYWYFDVWGQG | |
| | SLVTVSSASTKGPSVFPLAPSSKSTSGGTAALGCL | |
| | VKDYFPEPVTVSWNSGALTSGVHTFPAVLQSSG | |
| | LYSLSSVVTVPSSSLGTQTYICNVNHKPSNTKVD | |
| | KKVEPKSC | |
| PC23: LC | GGIDFCMLYNWPICAGGGGGSGGLSGRSDAGSP | 118 |
| | LGLAGSGGSDIQLTQSPSSLSASVGDRVSITCKAS | |
| | QDVSIAVAWYQQKPGKAPKLLIYSASYRYTGVP | |
| | DRFSGSGSGTDFTLTISSLQPEDFAVYYCQQHYI | |
| | TPLTFGAGTKVEIKRTVAAPSVFIFPPSDEQLKSG | |
| | TASVVCLLNNFYPREAKVQWKVDNALQSGNSQ | |
| | ESVTEQDSKDSTYSLSSTLTLSKADYEKHKVYA | |
| | CEVTHQGLSSPVTKSFNRGEC | |
| PC23: HC | EVQLVESGGGLVQPGNSLRLSCAASGFTFSKFG | 119 |
| | MSWVRQAPGKGLEWVSSISGSGRDTLYADSVK | |
| | GRFTISRDNAKTTLYLQMNSLRPEDTAVYYCTI | |
| | GGSLSVSSQGTLVTVSSGGGGSGGGSGGVYCGP | |
| | EFDESVGCMGGGGSGGGLSGRSDAGSPLGLAGS | |
| | GGGSEVQLVESGGGLVQPGGSLKLSCAASGFTF | |
| | NKYAMNWVRQAPGKGLEWVARIRSKYNNYAT | |
| | YYADSVKDRFTISRDDSKNTAYLQMNNLKTEDT | |
| | AVYYCVRHGNFGNSYISYWAYWGQGTLVTVS | |
| | SGGGGSGGGGSGGGGSQTVVTQEPSLTVSPGGT | |
| | VTLTCGSSTGAVTSGNYPNWVQQKPGQAPRGLI | |
| | GGTKFLAPGTPARFSGSLLGGKAALTLSGVQPE | |
| | DEAEYYCVLWYSNRWVFGGGTKLTVLGGGGS | |
| | QVQLQQSGSELKKPGASVKVSCKASGYTFTNY | |
| | GMNWVKQAPGQGLKWMGWINTYTGEPTYTD | |
| | DFKGRFAFSLDTSVSTAYLQISSLKADDTAVYFC | |
| | ARGGFGSSYWYFDVWGQGSLVTVSSASTKGPS | |
| | VFPLAPSSKSTSGGTAALGCLVKDYFPEPVTVSW | |
| | NSGALTSGVHTFPAVLQSSGLYSLSSVVTVPSSS | |
| | LGTQTYICNVNHKPSNTKVDKKVEPKSC | |
| PC24: LC | GGIDFCMLYNWPICAGGGGGSGGGGGSGGGGS | 120 |
| | GGASSGAGGSGGSDIQLTQSPSSLSASVGDRVSIT | |
| | CKASQDVSIAVAWYQQKPGKAPKLLIYSASYRY | |
| | TGVPDRFSGSGSGTDFTLTISSLQPEDFAVYYCQ | |
| | QHYITPLTFGAGTKVEIKRTVAAPSVFIFPPSDEQ | |
| | LKSGTASVVCLLNNFYPREAKVQWKVDNALQS | |
| | GNSQESVTEQDSKDSTYSLSSTLTLSKADYEKHK | |
| | VYACEVTHQGLSSPVTKSFNRGEC | |
| PC24: HC | EVQLVESGGGLVQPGNSLRLSCAASGFTFSKFG | 121 |
| | MSWVRQAPGKGLEWVSSISGSGRDTLYADSVK | |
| | GRFTISRDNAKTTLYLQMNSLRPEDTAVYYCTI | |
| | GGSLSVSSQGTLVTVSSGGGGSGGGSGGVYCGP | |
| | EFDESVGCMGGGGSGGGSGGGGSGGASSGAGG | |
| | SGGGSEVQLVESGGGLVQPGGSLKLSCAASGFT | |
| | FNKYAMNWVRQAPGKGLEWVARIRSKYNNYA | |
| | TYYADSVKDRFTISRDDSKNTAYLQMNNLKTED | |
| | TAVYYCVRHGNFGNSYISYWAYWGQGTLVTV | |
| | SSGGGGSGGGGSGGGGSQTVVTQEPSLTVSPGG | |
| | TVTLTCGSSTGAVTSGNYPNWVQQKPGQAPRG | |
| | LIGGTKFLAPGTPARFSGSLLGGKAALTLSGVQP | |
| | EDEAEYYCVLWYSNRWVFGGGTKLTVLGGGG | |
| | SQVQLQQSGSELKKPGASVKVSCKASGYTFTNY | |
| | GMNWVKQAPGQGLKWMGWINTYTGEPTYTD | |
| | DFKGRFAFSLDTSVSTAYLQISSLKADDTAVYFC | |
| | ARGGFGSSYWYFDVWGQGSLVTVSSASTKGPS | |
| | VFPLAPSSKSTSGGTAALGCLVKDYFPEPVTVSW | |
| | NSGALTSGVHTFPAVLQSSGLYSLSSVVTVPSSS | |
| | LGTQTYICNVNHKPSNTKVDKKVEPKSC | |
| PC25: LC | GGVDFCGLYHWPICYQGGGGSGGLSGRSDAGSP | 123 |
| | LGLAGSGGSDIQLTQSPSSLSASVGDRVSITCKAS | |
| | QDVSIAVAWYQQKPGKAPKLLIYSASYRYTGVP | |
| | DRFSGSGSGTDFTLTISSLQPEDFAVYYCQQHYIT | |
| | PLTFGAGTKVEIKRTVAAPSVFIFPPSDEQLKSGT | |
| | ASVVCLLNNFYPREAKVQWKVDNALQSGNSQE | |
| | SVTEQDSKDSTYSLSSTLTLSKADYEKHKVYAC | |
| | EVTHQGLSSPVTKSFNRGEC | |
| PC25: HC | EVQLVESGGGLVQPGGSLRLSCAASGSTFYTAV | 124 |
| | MGWVRQAPGKGLEWVAAIRWTALTTSYADSV | |
| | KGRFTISRDGAKTTLYLQMNSLRPEDTAVYYCA | |
| | ARGTLGLFTTADSYDYWGQGTLVTVSSGGGGS | |
| | GGGSGGVYCGPEFDESVGCMGGGGSGGGLSGR | |
| | SDAGSPLGLAGSGGGSEVQLVESGGGLVQPGGS | |
| | LKLSCAASGFTFNKYAMNWVRQAPGKGLEWV | |
| | ARIRSKYNNYATYYADSVKDRFTISRDDSKNTA | |
| | YLQMNNLKTEDTAVYYCVRHGNFGNSYISYWA | |
| | YWGQGTLVTVSSGGGGSGGGGSGGGGSQTVVT | |
| | QEPSLTVSPGGTVTLTCGSSTGAVTSGNYPNWV | |
| | QQKPGQAPRGLIGGTKFLAPGTPARFSGSLLGGK | |
| | AALTLSGVQPEDEAEYYCVLWYSNRWVFGGGT | |
| | KLTVLGGGGSQVQLQQSGSELKKPGASVKVSCK | |
| | ASGYTFTNYGMNWVKQAPGQGLKWMGWINTY | |
| | TGEPTYTDDFKGRFAFSLDTSVSTAYLQISSLKA | |
| | DDTAVYFCARGGFGSSYWYFDVWGQGSLVTVS | |
| | SASTKGPSVFPLAPSSKSTSGGTAALGCLVKDYF | |
| | PEPVTVSWNSGALTSGVHTFPAVLQSSGLYSLSS | |
| | VVTVPSSSLGTQTYICNVNHKPSNTKVDKKVEP | |
| | KSC | |
| PC26: LC | EWVAAIRWTALTTSYADSVKGRFTISRDGAKTT | 125 |
| | LYLQMNSLRPEDTAVYYCAARGTLGLFTTADSY | |
| | DYWGQGTLVTVSSGGGGSGGGSGGVYCGPEFD | |
| | ESVGCMGGGGSGGGLSGRSDAGSPLGLAGSGG | |
| | GSEVQLVESGGGLVQPGGSLKLSCAASGFTFNK | |
| | YAMNWVRQAPGKGLEWVARIRSKYNNYATYY | |
| | ADSVKDRFTISRDDSKNTAYLQMNNLKTEDTAV | |
| | YYCVRHGNFGNSYISYWAYWGQGTLVTVSSGG | |
| | GGSGGGGSGGGGSQTVVTQEPSLTVSPGGTVTL | |
| | TCGSSTGAVTSGNYPNWVQQKPGQAPRGLIGGT | |
| | KFLAPGTPARFSGSLLGGKAALTLSGVQPEDEAE | |
| | YYCVLWYSNRWVFGGGTKLTVLGGGGSDIQLT | |
| | QSPSSLSASVGDRVSITCKASQDVSIAVAWYQQK | |
| | PGKAPKLLIYSASYRYTGVPDRFSGSGSGTDFTL | |
| | TISSLQPEDFAVYYCQQHYITPLTFGAGTKVEIKR | |
| | TVAAPSVFIFPPSDEQLKSGTASVVCLLNNFYPRE | |
| | AKVQWKVDNALQSGNSQESVTEQDSKDSTYSL | |
| | SSTLTLSKADYEKHKVYACEVTHQGLSSPVTKSF | |
| | NRGEC | |
| PC26: HC | GGVDFCGLYHWPICYQGGGGSGGLSGRSDAGSP | 126 |
| | LGLAGSGGSQVQLQQSGSELKKPGASVKVSCKA | |
| | SGYTFTNYGMNWVKQAPGQGLKWMGWINTYT | |
| | GEPTYTDDFKGRFAFSLDTSVSTAYLQISSLKAD | |
| | DTAVYFCARGGFGSSYWYFDVWGQGSLVTVSS | |
| | ASTKGPSVFPLAPSSKSTSGGTAALGCLVKDYFP | |
| | EPVTVSWNSGALTSGVHTFPAVLQSSGLYSLSSV | |
| | VTVPSSSLGTQTYICNVNHKPSNTKVDKKVEPKS | |
| | C | |
| PC27: LC | GGVDFCGLYHWPICYQGGGGSSGGSAAGLLAPP | 127 |
| | GGLSGRSDAGGGGSDIQLTQSPSSLSASVGDRVS | |
| | ITCKASQDVSIAVAWYQQKPGKAPKLLIYSASYR | |
| | YTGVPDRFSGSGSGTDFTLTISSLQPEDFAVYYC | |
| | QQHYITPLTFGAGTKVEIKRTVAAPSVFIFPPSDE | |
| | QLKSGTASVVCLLNNFYPREAKVQWKVDNALQ | |
| | SGNSQESVTEQDSKDSTYSLSSTLTLSKADYEKH | |
| | KVYACEVTHQGLSSPVTKSFNRGEC | |
| PC27: HC | EVQLVESGGGLVQPGGSLRLSCAASGSTFYTAV | 128 |
| | MGWVRQAPGKGLEWVAAIRWTALTTSYADSV | |
| | KGRFTISRDGAKTTLYLQMNSLRPEDTAVYYCA | |
| | ARGTLGLFTTADSYDYWGQGTLVTVSSGGGGS | |
| | GGGSGGVYCGPEFDESVGCMGSSGGSAAGLLAP | |
| | PGGLSGRSDAGGGGSEVQLVESGGGLVQPGGSL | |
| | KLSCAASGFTFNKYAMNWVRQAPGKGLEWVA | |
| | RIRSKYNNYATYYADSVKDRFTISRDDSKNTAY | |
| | LQMNNLKTEDTAVYYCVRHGNFGNSYISYWAY | |
| | WGQGTLVTVSSGGGGSGGGGSGGGGSQTVVTQ | |
| | EPSLTVSPGGTVTLTCGSSTGAVTSGNYPNWVQ | |
| | QKPGQAPRGLIGGTKFLAPGTPARFSGSLLGGKA | |
| | ALTLSGVQPEDEAEYYCVLWYSNRWVFGGGTK | |
| | LTVLGGGGSQVQLQQSGSELKKPGASVKVSCKA | |
| | SGYTFTNYGMNWVKQAPGQGLKWMGWINTYT | |
| | GEPTYTDDFKGRFAFSLDTSVSTAYLQISSLKAD | |
| | DTAVYFCARGGFGSSYWYFDVWGQGSLVTVSS | |
| | ASTKGPSVFPLAPSSKSTSGGTAALGCLVKDYFP | |
| | EPVTVSWNSGALTSGVHTFPAVLQSSGLYSLSSV | |
| | VTVPSSSLGTQTYICNVNHKPSNTKVDKKVEPKS | |
| | C | |
| PC28: LC | EVQLVESGGGLVQPGGSLRLSCAASGSTFYTAV | 129 |
| | MGWVRQAPGKGLEWVAAIRWTALTTSYADSV | |
| | KGRFTISRDGAKTTLYLQMNSLRPEDTAVYYCA | |
| | ARGTLGLFTTADSYDYWGQGTLVTVSSGGGGS | |
| | GGGSGGVYCGPEFDESVGCMGSSGGSAAGLLAP | |
| | PGGLSGRSDAGGGGSEVQLVESGGGLVQPGGSL | |
| | KLSCAASGFTFNKYAMNWVRQAPGKGLEWVA | |
| | RIRSKYNNYATYYADSVKDRFTISRDDSKNTAY | |
| | LQMNNLKTEDTAVYYCVRHGNFGNSYISYWAY | |
| | WGQGTLVTVSSGGGGSGGGGSGGGGSQTVVTQ | |
| | EPSLTVSPGGTVTLTCGSSTGAVTSGNYPNWVQ | |
| | QKPGQAPRGLIGGTKFLAPGTPARFSGSLLGGKA | |
| | ALTLSGVQPEDEAEYYCVLWYSNRWVFGGGTK | |
| | LTVLGGGGSDIQLTQSPSSLSASVGDRVSITCKAS | |
| | QDVSIAVAWYQQKPGKAPKLLIYSASYRYTGVP | |
| | DRFSGSGSGTDFTLTISSLQPEDFAVYYCQQHYIT | |
| | PLTFGAGTKVEIKRTVAAPSVFIFPPSDEQLKSGT | |
| | ASVVCLLNNFYPREAKVQWKVDNALQSGNSQE | |
| | SVTEQDSKDSTYSLSSTLTLSKADYEKHKVYAC | |
| | EVTHQGLSSPVTKSFNRGEC | |
| PC28: HC | GGVDFCGLYHWPICYQGGGGSSGGSAAGLLAPP | 130 |
| | GGLSGRSDAGGGGSQVQLQQSGSELKKPGASVK | |
| | VSCKASGYTFTNYGMNWVKQAPGQGLKWMG | |
| | WINTYTGEPTYTDDFKGRFAFSLDTSVSTAYLQI | |
| | SSLKADDTAVYFCARGGFGSSYWYFDVWGQGS | |
| | LVTVSSASTKGPSVFPLAPSSKSTSGGTAALGCL | |
| | VKDYFPEPVTVSWNSGALTSGVHTFPAVLQSSG | |
| | LYSLSSVVTVPSSSLGTQTYICNVNHKPSNTKVD | |
| | KKVEPKSC | |
| PC29: LC | GGVDFCGLYHWPICYQGGGGSGGGGGSGGGGS | 241 |
| | GGASSGAGGSDIQLTQSPSSLSASVGDRVSITCK | |
| | ASQDVSIAVAWYQQKPGKAPKLLIYSASYRYTG | |
| | VPDRFSGSGSGTDFTLTISSLQPEDFAVYYCQQH | |
| | YITPLTFGAGTKVEIKRTVAAPSVFIFPPSDEQLK | |
| | SGTASVVCLLNNFYPREAKVQWKVDNALQSGN | |
| | SQESVTEQDSKDSTYSLSSTLTLSKADYEKHKVY | |
| | ACEVTHQGLSSPVTKSFNRGEC | |
| PC29: HC | EVQLVESGGGLVQPGNSLRLSCAASGFTFSKFG | 131 |
| | MSWVRQAPGKGLEWVSSISGSGRDTLYADSVK | |
| | GRFTISRDNAKTTLYLQMNSLRPEDTAVYYCTIG | |
| | GSLSVSSQGTLVTVSSGGGGSGGGSGGVYCGPE | |
| | FDESVGCMGGGGSGGGSGGGGSGGASSGAGGS | |
| | GGGSEVQLVESGGGLVQPGGSLKLSCAASGFTF | |
| | NKYAMNWVRQAPGKGLEWVARIRSKYNNYAT | |
| | YYADSVKDRFTISRDDSKNTAYLQMNNLKTEDT | |
| | AVYYCVRHGNFGNSYISYWAYWGQGTLVTVSS | |
| | GGGGSGGGGSGGGGSQTVVTQEPSLTVSPGGTV | |
| | TLTCGSSTGAVTSGNYPNWVQQKPGQAPRGLIG | |
| | GTKFLAPGTPARFSGSLLGGKAALTLSGVQPEDE | |
| | AEYYCVLWYSNRWVFGGGTKLTVLGGGGSQV | |
| | QLQQSGSELKKPGASVKVSCKASGYTFTNYGM | |
| | NWVKQAPGQGLKWMGWINTYTGEPTYTDDFK | |
| | GRFAFSLDTSVSTAYLQISSLKADDTAVYFCARG | |
| | GFGSSYWYFDVWGQGSLVTVSSASTKGPSVFPL | |
| | APSSKSTSGGTAALGCLVKDYFPEPVTVSWNSG | |
| | ALTSGVHTFPAVLQSSGLYSLSSVVTVPSSSLGT | |
| | QTYICNVNHKPSNTKVDKKVEPKSC | |
| PC30: LC | EVQLVESGGGLVQPGNSLRLSCAASGFTFSKFG | 132 |
| | MSWVRQAPGKGLEWVSSISGSGRDTLYADSVK | |
| | GRFTISRDNAKTTLYLQMNSLRPEDTAVYYCTIG | |
| | GSLSVSSQGTLVTVSSGGGGSGGGSGGVYCGPE | |
| | FDESVGCMGGGGSGGGSGGGGSGGASSGAGGS | |
| | GGGSEVQLVESGGGLVQPGGSLKLSCAASGFTF | |
| | NKYAMNWVRQAPGKGLEWVARIRSKYNNYAT | |
| | YYADSVKDRFTISRDDSKNTAYLQMNNLKTEDT | |
| | AVYYCVRHGNFGNSYISYWAYWGQGTLVTVSS | |
| | GGGGSGGGGSGGGGSQTVVTQEPSLTVSPGGTV | |
| | TLTCGSSTGAVTSGNYPNWVQQKPGQAPRGLIG | |
| | GTKFLAPGTPARFSGSLLGGKAALTLSGVQPEDE | |
| | AEYYCVLWYSNRWVFGGGTKLTVLGGGGSDIQ | |
| | LTQSPSSLSASVGDRVSITCKASQDVSIAVAWYQ | |
| | QKPGKAPKLLIYSASYRYTGVPDRFSGSGSGTDF | |
| | TLTISSLQPEDFAVYYCQQHYITPLTFGAGTKVEI | |
| | KRTVAAPSVFIFPPSDEQLKSGTASVVCLLNNFY | |
| | PREAKVQWKVDNALQSGNSQESVTEQDSKDST | |
| | YSLSSTLTLSKADYEKHKVYACEVTHQGLSSPV | |
| | TKSFNRGEC | |
| PC30: HC | GGVDFCGLYHWPICYQGGGGSGGGGGSGGGGS | 242 |
| | GGASSGAGGSQVQLQQSGSELKKPGASVKVSCK | |
| | ASGYTFTNYGMNWVKQAPGQGLKWMGWINTY | |
| | TGEPTYTDDFKGRFAFSLDTSVSTAYLQISSLKA | |
| | DDTAVYFCARGGFGSSYWYFDVWGQGSLVTVS | |
| | SASTKGPSVFPLAPSSKSTSGGTAALGCLVKDYF | |
| | PEPVTVSWNSGALTSGVHTFPAVLQSSGLYSLSS | |
| | VVTVPSSSLGTQTYICNVNHKPSNTKVDKKVEP | |
| | KSC | |

In some embodiments, the isolated polypeptide or polypeptide complex comprises amino acid sequences according to SEQ ID NO: 76 and SEQ ID NO: 77. In some embodiments, the isolated polypeptide or polypeptide complex comprises amino acid sequences with at least 90% sequence identity to SEQ ID NO: 76 and SEQ ID NO: 77. In some embodiments, the isolated polypeptide or polypeptide complex comprises amino acid sequences with at least 95% sequence identity to SEQ ID NO: 76 and SEQ ID NO: 77. In some embodiments, the isolated polypeptide or polypeptide complex comprises amino acid sequences with at least 99% sequence identity to SEQ ID NO: 76 and SEQ ID NO: 77.

In some embodiments, the isolated polypeptide or polypeptide complex comprises amino acid sequences according to SEQ ID NO: 78 and SEQ ID NO: 79. In some embodiments, the isolated polypeptide or polypeptide complex comprises amino acid sequences with at least 90% sequence identity to SEQ ID NO: 78 and SEQ ID NO: 79. In some embodiments, the isolated polypeptide or polypeptide complex comprises amino acid sequences with at least 95% sequence identity to SEQ ID NO: 78 and SEQ ID NO: 79. In some embodiments, the isolated polypeptide or polypeptide complex comprises amino acid sequences with at least 99% sequence identity to SEQ ID NO: 78 and SEQ ID NO: 79.

In some embodiments, the isolated polypeptide or polypeptide complex comprises amino acid sequences according to SEQ ID NO: 80 and SEQ ID NO: 81. In some embodiments, the isolated polypeptide or polypeptide complex comprises amino acid sequences with at least 90% sequence identity to SEQ ID NO: 80 and SEQ ID NO: 81. In some embodiments, the isolated polypeptide or polypeptide complex comprises amino acid sequences with at least 95% sequence identity to SEQ ID NO: 80 and SEQ ID NO: 81. In some embodiments, the isolated polypeptide or polypeptide complex comprises amino acid sequences with at least 99% sequence identity to SEQ ID NO: 80 and SEQ ID NO: 81.

In some embodiments, the isolated polypeptide or polypeptide complex comprises amino acid sequences according to SEQ ID NO: 82 and SEQ ID NO: 83. In some embodiments, the isolated polypeptide or polypeptide complex comprises amino acid sequences with at least 90% sequence identity to SEQ ID NO: 82 and SEQ ID NO: 83. In some embodiments, the isolated polypeptide or polypeptide complex comprises amino acid sequences with at least 95% sequence identity to SEQ ID NO: 82 and SEQ ID NO: 83. In some embodiments, the isolated polypeptide or polypeptide complex comprises amino acid sequences with at least 99% sequence identity to SEQ ID NO: 82 and SEQ ID NO: 83.

In some embodiments, the isolated polypeptide or polypeptide complex comprises amino acid sequences according to SEQ ID NO: 84 and SEQ ID NO: 85. In some embodiments, the isolated polypeptide or polypeptide complex comprises amino acid sequences with at least 90% sequence identity to SEQ ID NO: 84 and SEQ ID NO: 85. In some embodiments, the isolated polypeptide or polypeptide complex comprises amino acid sequences with at least 95% sequence identity to SEQ ID NO: 84 and SEQ ID NO: 85. In some embodiments, the isolated polypeptide or polypeptide complex comprises amino acid sequences with at least 99% sequence identity to SEQ ID NO: 84 and SEQ ID NO: 85.

In some embodiments, the isolated polypeptide or polypeptide complex comprises amino acid sequences according to SEQ ID NO: 86 and SEQ ID NO: 87. In some embodiments, the isolated polypeptide or polypeptide complex comprises amino acid sequences with at least 90% sequence identity to SEQ ID NO: 86 and SEQ ID NO: 87. In some embodiments, the isolated polypeptide or polypeptide complex comprises amino acid sequences with at least 95% sequence identity to SEQ ID NO: 86 and SEQ ID NO: 87. In some embodiments, the isolated polypeptide or polypeptide complex comprises amino acid sequences with at least 99% sequence identity to SEQ ID NO: 86 and SEQ ID NO: 87.

In some embodiments, the isolated polypeptide or polypeptide complex comprises amino acid sequences according to SEQ ID NO: 88 and SEQ ID NO: 89. In some embodiments, the isolated polypeptide or polypeptide complex comprises amino acid sequences with at least 90% sequence identity to SEQ ID NO: 88 and SEQ ID NO: 89. In some embodiments, the isolated polypeptide or polypeptide complex comprises amino acid sequences with at least 95% sequence identity to SEQ ID NO: 88 and SEQ ID NO: 89. In some embodiments, the isolated polypeptide or polypeptide complex comprises amino acid sequences with at least 99% sequence identity to SEQ ID NO: 88 and SEQ ID NO: 89.

In some embodiments, the isolated polypeptide or polypeptide complex comprises amino acid sequences according to SEQ ID NO: 90 and SEQ ID NO: 91. In some embodiments, the isolated polypeptide or polypeptide complex comprises amino acid sequences with at least 90% sequence identity to SEQ ID NO: 90 and SEQ ID NO: 91. In some embodiments, the isolated polypeptide or polypeptide complex comprises amino acid sequences with at least 95% sequence identity to SEQ ID NO: 90 and SEQ ID NO: 91. In some embodiments, the isolated polypeptide or polypeptide complex comprises amino acid sequences with at least 99% sequence identity to SEQ ID NO: 90 and SEQ ID NO: 91.

In some embodiments, the isolated polypeptide or polypeptide complex comprises amino acid sequences according to SEQ ID NO: 92 and SEQ ID NO: 93. In some embodiments, the isolated polypeptide or polypeptide complex comprises amino acid sequences with at least 90% sequence identity to SEQ ID NO: 92 and SEQ ID NO: 93. In some embodiments, the isolated polypeptide or polypeptide complex comprises amino acid sequences with at least 95% sequence identity to SEQ ID NO: 92 and SEQ ID NO: 93. In some embodiments, the isolated polypeptide or polypeptide complex comprises amino acid sequences with at least 99% sequence identity to SEQ ID NO: 92 and SEQ ID NO: 93.

In some embodiments, the isolated polypeptide or polypeptide complex comprises amino acid sequences according to SEQ ID NO: 94 and SEQ ID NO: 95. In some embodiments, the isolated polypeptide or polypeptide complex comprises amino acid sequences with at least 90% sequence identity to SEQ ID NO: 94 and SEQ ID NO: 95. In some embodiments, the isolated polypeptide or polypeptide complex comprises amino acid sequences with at least 95% sequence identity to SEQ ID NO: 94 and SEQ ID NO: 95. In some embodiments, the isolated polypeptide or polypeptide complex comprises amino acid sequences with at least 99% sequence identity to SEQ ID NO: 94 and SEQ ID NO: 95.

In some embodiments, the isolated polypeptide or polypeptide complex comprises amino acid sequences according to SEQ ID NO: 96 and SEQ ID NO: 97. In some embodiments, the isolated polypeptide or polypeptide complex comprises amino acid sequences with at least 90% sequence identity to SEQ ID NO: 96 and SEQ ID NO: 97. In some embodiments, the isolated polypeptide or polypeptide complex comprises amino acid sequences with at least 95% sequence identity to SEQ ID NO: 96 and SEQ ID NO: 97. In some embodiments, the isolated polypeptide or polypeptide complex comprises amino acid sequences with at least 99% sequence identity to SEQ ID NO: 96 and SEQ ID NO: 97.

In some embodiments, the isolated polypeptide or polypeptide complex comprises amino acid sequences according to SEQ ID NO: 98 and SEQ ID NO: 99. In some embodiments, the isolated polypeptide or polypeptide complex comprises amino acid sequences with at least 90% sequence identity to SEQ ID NO: 98 and SEQ ID NO: 99. In some embodiments, the isolated polypeptide or polypeptide complex comprises amino acid sequences with at least 95% sequence identity to SEQ ID NO: 98 and SEQ ID NO: 99. In some embodiments, the isolated polypeptide or polypeptide complex comprises amino acid sequences with at least 99% sequence identity to SEQ ID NO: 98 and SEQ ID NO: 99.

In some embodiments, the isolated polypeptide or polypeptide complex comprises amino acid sequences according to SEQ ID NO: 100 and SEQ ID NO: 101. In some embodiments, the isolated polypeptide or polypeptide complex comprises amino acid sequences with at least 90% sequence identity to SEQ ID NO: 100 and SEQ ID NO: 101. In some embodiments, the isolated polypeptide or polypeptide complex comprises amino acid sequences with at least 95% sequence identity to SEQ ID NO: 100 and SEQ ID NO: 101. In some embodiments, the isolated polypeptide or polypeptide complex comprises amino acid sequences with at least 99% sequence identity to SEQ ID NO: 100 and SEQ ID NO: 101.

In some embodiments, the isolated polypeptide or polypeptide complex comprises amino acid sequences according to SEQ ID NO: 102 and SEQ ID NO: 103. In some embodiments, the isolated polypeptide or polypeptide complex comprises amino acid sequences with at least 90% sequence identity to SEQ ID NO: 102 and SEQ ID NO: 103. In some embodiments, the isolated polypeptide or polypeptide complex comprises amino acid sequences with at least 95% sequence identity to SEQ ID NO: 102 and SEQ ID NO: 103. In some embodiments, the isolated polypeptide or polypeptide complex comprises amino acid sequences with at least 99% sequence identity to SEQ ID NO: 102 and SEQ ID NO: 103.

In some embodiments, the isolated polypeptide or polypeptide complex comprises amino acid sequences according to SEQ ID NO: 104 and SEQ ID NO: 105. In some embodiments, the isolated polypeptide or polypeptide complex comprises amino acid sequences with at least 90% sequence identity to SEQ ID NO: 104 and SEQ ID NO: 105. In some embodiments, the isolated polypeptide or polypeptide complex comprises amino acid sequences with at least 95% sequence identity to SEQ ID NO: 104 and SEQ ID NO: 105. In some embodiments, the isolated polypeptide or polypeptide complex comprises amino acid sequences with at least 99% sequence identity to SEQ ID NO: 104 and SEQ ID NO: 105.

In some embodiments, the isolated polypeptide or polypeptide complex comprises amino acid sequences according to SEQ ID NO: 106 and SEQ ID NO: 107. In some embodiments, the isolated polypeptide or polypeptide complex comprises amino acid sequences with at least 90% sequence identity to SEQ ID NO: 106 and SEQ ID NO: 107. In some embodiments, the isolated polypeptide or polypeptide complex comprises amino acid sequences with at least 95% sequence identity to SEQ ID NO: 106 and SEQ ID NO: 107. In some embodiments, the isolated polypeptide or polypeptide complex comprises amino acid sequences with at least 99% sequence identity to SEQ ID NO: 106 and SEQ ID NO: 107.

In some embodiments, the isolated polypeptide or polypeptide complex comprises amino acid sequences according to SEQ ID NO: 108 and SEQ ID NO: 109. In some embodiments, the isolated polypeptide or polypeptide complex comprises amino acid sequences with at least 90% sequence identity to SEQ ID NO: 108 and SEQ ID NO: 109. In some embodiments, the isolated polypeptide or polypeptide complex comprises amino acid sequences with at least 95% sequence identity to SEQ ID NO: 108 and SEQ ID NO: 109. In some embodiments, the isolated polypeptide or polypeptide complex comprises amino acid sequences with at least 99% sequence identity to SEQ ID NO: 108 and SEQ ID NO: 109.

In some embodiments, the isolated polypeptide or polypeptide complex comprises amino acid sequences according to SEQ ID NO: 110 and SEQ ID NO: 111. In some embodiments, the isolated polypeptide or polypeptide complex comprises amino acid sequences with at least 90% sequence identity to SEQ ID NO: 110 and SEQ ID NO: 111. In some embodiments, the isolated polypeptide or polypeptide complex comprises amino acid sequences with at least 95% sequence identity to SEQ ID NO: 110 and SEQ ID NO: 111. In some embodiments, the isolated polypeptide or polypeptide complex comprises amino acid sequences with at least 99% sequence identity to SEQ ID NO: 110 and SEQ ID NO: 111.

In some embodiments, the isolated polypeptide or polypeptide complex comprises amino acid sequences according to SEQ ID NO: 112 and SEQ ID NO: 113. In some embodiments, the isolated polypeptide or polypeptide complex comprises amino acid sequences with at least 90% sequence identity to SEQ ID NO: 112 and SEQ ID NO: 113. In some embodiments, the isolated polypeptide or polypeptide complex comprises amino acid sequences with at least 95% sequence identity to SEQ ID NO: 112 and SEQ ID NO: 113. In some embodiments, the isolated polypeptide or polypeptide complex comprises amino acid sequences with at least 99% sequence identity to SEQ ID NO: 112 and SEQ ID NO: 113.

In some embodiments, the isolated polypeptide or polypeptide complex comprises amino acid sequences according to SEQ ID NO: 114 and SEQ ID NO: 115. In some embodiments, the isolated polypeptide or polypeptide complex comprises amino acid sequences with at least 90% sequence identity to SEQ ID NO: 114 and SEQ ID NO: 115. In some embodiments, the isolated polypeptide or polypeptide complex comprises amino acid sequences with at least 95% sequence identity to SEQ ID NO: 114 and SEQ ID NO: 115. In some embodiments, the isolated polypeptide or polypeptide complex comprises amino acid sequences with at least 99% sequence identity to SEQ ID NO: 114 and SEQ ID NO: 115.

In some embodiments, the isolated polypeptide or polypeptide complex comprises amino acid sequences according to SEQ ID NO: 116 and SEQ ID NO: 117. In some embodiments, the isolated polypeptide or polypeptide complex comprises amino acid sequences with at least 90% sequence identity to SEQ ID NO: 116 and SEQ ID NO: 117. In some embodiments, the isolated polypeptide or polypeptide complex comprises amino acid sequences with at least 95% sequence identity to SEQ ID NO: 116 and SEQ ID NO: 117. In some embodiments, the isolated polypeptide or polypeptide complex comprises amino acid sequences with at least 99% sequence identity to SEQ ID NO: 116 and SEQ ID NO: 117.

In some embodiments, the isolated polypeptide or polypeptide complex comprises amino acid sequences according to SEQ ID NO: 118 and SEQ ID NO: 119. In some embodiments, the isolated polypeptide or polypeptide complex comprises amino acid sequences with at least 90% sequence identity to SEQ ID NO: 118 and SEQ ID NO: 119. In some embodiments, the isolated polypeptide or polypeptide complex comprises amino acid sequences with at least 95% sequence identity to SEQ ID NO: 118 and SEQ ID NO: 119. In some embodiments, the isolated polypeptide or polypeptide complex comprises amino acid sequences with at least 99% sequence identity to SEQ ID NO: 118 and SEQ ID NO: 119.

In some embodiments, the isolated polypeptide or polypeptide complex comprises amino acid sequences according to SEQ ID NO: 120 and SEQ ID NO: 121. In some embodiments, the isolated polypeptide or polypeptide complex comprises amino acid sequences with at least 90% sequence identity to SEQ ID NO: 120 and SEQ ID NO: 121. In some embodiments, the isolated polypeptide or polypeptide complex comprises amino acid sequences with at least 95% sequence identity to SEQ ID NO: 120 and SEQ ID NO: 121. In some embodiments, the isolated polypeptide or polypeptide complex comprises amino acid sequences with at least 99% sequence identity to SEQ ID NO: 120 and SEQ ID NO: 121.

In some embodiments, the isolated polypeptide or polypeptide complex comprises amino acid sequences according to SEQ ID NO: 123 and SEQ ID NO: 124. In some embodiments, the isolated polypeptide or polypeptide complex comprises amino acid sequences with at least 90% sequence identity to SEQ ID NO: 123 and SEQ ID NO: 124. In some embodiments, the isolated polypeptide or polypeptide complex comprises amino acid sequences with at least 95% sequence identity to SEQ ID NO: 123 and SEQ ID NO: 124. In some embodiments, the isolated polypeptide or polypeptide complex comprises amino acid sequences with at least 99% sequence identity to SEQ ID NO: 123 and SEQ ID NO: 124.

In some embodiments, the isolated polypeptide or polypeptide complex comprises amino acid sequences according to SEQ ID NO: 125 and SEQ ID NO: 126. In some embodiments, the isolated polypeptide or polypeptide complex comprises amino acid sequences with at least 90% sequence identity to SEQ ID NO: 125 and SEQ ID NO: 126. In some embodiments, the isolated polypeptide or polypeptide complex comprises amino acid sequences with at least 95% sequence identity to SEQ ID NO: 125 and SEQ ID NO: 126. In some embodiments, the isolated polypeptide or polypeptide complex comprises amino acid sequences with at least 99% sequence identity to SEQ ID NO: 125 and SEQ ID NO: 126.

In some embodiments, the isolated polypeptide or polypeptide complex comprises amino acid sequences according to SEQ ID NO: 127 and SEQ ID NO: 128. In some embodiments, the isolated polypeptide or polypeptide complex comprises amino acid sequences with at least 90% sequence identity to SEQ ID NO: 127 and SEQ ID NO: 128. In some embodiments, the isolated polypeptide or polypeptide complex comprises amino acid sequences with at least 95% sequence identity to SEQ ID NO: 127 and SEQ ID NO: 128. In some embodiments, the isolated polypeptide or polypeptide complex comprises amino acid sequences with at least 99% sequence identity to SEQ ID NO: 127 and SEQ ID NO: 128.

In some embodiments, the isolated polypeptide or polypeptide complex comprises amino acid sequences according to SEQ ID NO: 129 and SEQ ID NO: 130. In some embodiments, the isolated polypeptide or polypeptide complex comprises amino acid sequences with at least 90% sequence identity to SEQ ID NO: 129 and SEQ ID NO: 130. In some embodiments, the isolated polypeptide or polypeptide complex comprises amino acid sequences with at least 95% sequence identity to SEQ ID NO: 129 and SEQ ID NO: 130. In some embodiments, the isolated polypeptide or polypeptide complex comprises amino acid sequences with at least 99% sequence identity to SEQ ID NO: 129 and SEQ ID NO: 130.

In some embodiments, the isolated polypeptide or polypeptide complex comprises amino acid sequences according to SEQ ID NO: 241 and SEQ ID NO: 131. In some embodiments, the isolated polypeptide or polypeptide complex comprises amino acid sequences with at least 90% sequence identity to SEQ ID NO: 241 and SEQ ID NO: 131. In some embodiments, the isolated polypeptide or polypeptide complex comprises amino acid sequences with at least 95% sequence identity to SEQ ID NO: 241 and SEQ ID NO: 131. In some embodiments, the isolated polypeptide or polypeptide complex comprises amino acid sequences with at least 99% sequence identity to SEQ ID NO: 241 and SEQ ID NO: 131.

In some embodiments, the isolated polypeptide or polypeptide complex comprises amino acid sequences according to SEQ ID NO: 132 and SEQ ID NO: 242. In some embodiments, the isolated polypeptide or polypeptide complex comprises amino acid sequences with at least 90% sequence identity to SEQ ID NO: 132 and SEQ ID NO: 242. In some embodiments, the isolated polypeptide or polypeptide complex comprises amino acid sequences with at least 95% sequence identity to SEQ ID NO: 132 and SEQ ID NO: 242. In some embodiments, the isolated polypeptide or polypeptide complex comprises amino acid sequences with at least 99% sequence identity to SEQ ID NO: 132 and SEQ ID NO: 242.

Polypeptides or polypeptide complexes, in some embodiments, comprise a sequence set forth in Table 6. In some embodiments, the sequence comprises at least or about 70%, 80%, 85%, 90%, 91%, 92%, 93%, 94%, 95%, 96%, 97%, 98%, 99%, or 100% sequence identity to any one of SEQ ID NOs: 74-132, 241-242. In some instances, the sequence comprises at least or about 95% homology to SEQ ID NOs: 74-132, 241-242. In some instances, the sequence comprises at least or about 97% homology to SEQ ID NOs: 74-132, 241-242. In some instances, the sequence comprises at least or about 99% homology to SEQ ID NOs: 74-132, 241-242. In some instances, the sequence comprises at least or about 100% homology to SEQ ID NOs: 74-132, 241-242. In some instances, the sequence comprises at least a portion having at least or about 10, 20, 30, 40, 50, 60, 70, 80, 90, 100, 110, 120, 130, 140, 150, 160, 170, 180, 190, 200, 210, or more than 210 amino acids of any one of SEQ ID NOs: 74, 77, 78, 81, 82, 84, 87, 89, 90, 93, 94, 97, 98, 100, 103, 105, 106, 108, 111, 113, 114, 116, 118, 120, 123, 126, 127, 130, 241, or 242. In some instances, the sequence comprises at least a portion having at least or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, or more than 450 amino acids of any one of SEQ ID NOs: 75, 76, 79, or 80. In some instances, the sequence comprises at least a portion having at least or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, or more than 640 amino acids of any one of SEQ ID NOs: 83, 85, 86, 88, 91, 92, 95, 96, 99, 101, 102, 104, 107, 109, 112, 115, 117, 119, 121, 124, 125, 128, 129, 131, or 132.

As used herein, the term “percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

Disclosed herein, in some embodiments, are isolated polypeptides or polypeptide complexes comprising a structural arrangement according to Configuration 1:

wherein the polypeptide complex comprises a single chain variable fragment (scFv) comprising a light chain variable domain and a heavy chain variable domain, wherein the scFv further comprises a peptide that impairs binding of the scFv to an effector cell antigen and the peptide is linked to a N-terminus of the heavy chain variable domain of the scFv with a linking moiety that is a substrate for a tumor specific protease, and the peptide further comprises a half-life extending molecule; and a Fab that binds to TROP2, wherein the Fab comprises a Fab light chain polypeptide and a Fab heavy chain polypeptide, wherein the Fab heavy chain polypeptide is linked to a C terminus of the light chain variable domain of the scFv, and wherein the Fab further comprises P₂ and L₂, wherein P₂ comprises a peptide that impairs binding to TROP2; and L₂ comprises a linking moiety that connects the Fab light chain polypeptide to P₂ and is a substrate for a tumor specific protease.

Disclosed herein, in some embodiments, are isolated polypeptides or polypeptide complexes comprising a structural arrangement according to Configuration 2:

wherein the polypeptide complex comprises a single chain variable fragment (scFv) comprising a light chain variable domain and a heavy chain variable domain, wherein the scFv further comprises a peptide that impairs binding of the scFv to an effector cell antigen and the peptide is linked to a N-terminus of the heavy chain variable domain of the scFv with a linking moiety that is a substrate for a tumor specific protease, and the peptide further comprises a half-life extending molecule; and a Fab that binds to TROP2, wherein the Fab comprises a Fab light chain polypeptide and a Fab heavy chain polypeptide, wherein the Fab light chain polypeptide is linked to a C terminus of the light chain variable domain of the scFv, and wherein the Fab further comprises P₂ and L₂, wherein P₂ comprises a peptide that impairs binding to TROP2; and L₂ comprises a linking moiety that connects the Fab heavy chain polypeptide to P₂ and is a substrate for a tumor specific protease.

Disclosed herein, in some embodiments, are isolated polypeptides or polypeptide complexes comprising a structural arrangement according to Configuration 3:

wherein the isolated polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to the light chain variable domain of the scFv with a linking moiety that is a substrate for a tumor specific protease, and the peptide is further linked to a half-life extending molecule; and a Fab that binds to TROP2, wherein the Fab comprises a Fab light chain polypeptide chain and a Fab heavy chain polypeptide chain, and wherein the Fab heavy chain polypeptide chain is linked to a C terminus of the heavy chain variable domain of the scFv.

Disclosed herein, in some embodiments, are isolated polypeptides or polypeptide complexes comprising a structural arrangement according to Configuration 4:

wherein the isolated polypeptide or polypeptide complex comprises a single chain variable fragment (scFv) comprising a light chain variable domain and a heavy chain variable domain, wherein the scFv is linked to a peptide (P₁) that impairs binding of the scFv to an effector cell antigen and P₁ is linked to a N-terminus of the light chain variable domain of the scFv with a linking moiety that is a substrate for a tumor specific protease, and P₁ is further linked to a half-life extending molecule; and a Fab that binds to TROP2, wherein the Fab comprises a Fab light chain polypeptide and a Fab heavy chain polypeptide, wherein the Fab light chain polypeptide is linked to a C terminus of the heavy chain variable domain of the scFv, and wherein the Fab is linked to P₂ and L₂, wherein P₂ comprises a peptide that impairs binding to TROP2; and L₂ comprises a linking moiety that connects the Fab heavy chain polypeptide to P₂ and is a substrate for a tumor specific protease.

Disclosed herein, in some embodiments, are isolated polypeptides or polypeptide complexes comprising a structural arrangement according to Configuration 5:

wherein the isolated polypeptide or polypeptide complex comprises a single chain variable fragment (scFv) comprising a light chain variable domain and a heavy chain variable domain, wherein the scFv is further linked to a peptide that impairs binding of the scFv to an effector cell antigen and the peptide is linked to a N-terminus of the light chain variable domain of the scFv with a linking moiety that is a substrate for a tumor specific protease, and the peptide is further linked to a half-life extending molecule; and a Fab that binds to TROP2, wherein the Fab comprises a Fab light chain polypeptide and a Fab heavy chain polypeptide, wherein the Fab light chain polypeptide is linked to a C terminus of the heavy chain variable domain of the scFv.

Disclosed herein, in some embodiments, are isolated polypeptides or polypeptide complexes comprising a structural arrangement according to Configuration 6:

wherein the isolated polypeptide or polypeptide complex comprises a single chain variable fragment (scFv) comprising a light chain variable domain and a heavy chain variable domain, wherein the scFv is linked to a peptide (P₁) that impairs binding of the scFv to an effector cell antigen and P₁ is linked to a N-terminus of the heavy chain variable domain of the scFv with a linking moiety (L₁) that is a substrate for a tumor specific protease, and P₁ is further linked to a half-life extending molecule; and a Fab that binds to TROP2, wherein the Fab comprises a Fab light chain polypeptide and a Fab heavy chain polypeptide, wherein the Fab heavy chain polypeptide is linked to a C terminus of the light chain variable domain of the scFv, and wherein the Fab is linked to P₂ and L₂, wherein P₂ comprises a peptide that impairs binding to TROP2; and L₂ comprises a linking moiety that connects the Fab light chain polypeptide to P₂ and is a substrate for a tumor specific protease.

Disclosed herein, in some embodiments, are isolated polypeptides or polypeptide complexes comprising a structural arrangement according to Configuration 7:

wherein the isolated polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to the heavy chain variable domain of the scFv with a linking moiety that is a substrate for a tumor specific protease, and the peptide is further linked to a half-life extending molecule; and a Fab that binds to TROP2, wherein the Fab comprises a Fab light chain polypeptide chain and a Fab heavy chain polypeptide chain, and wherein the Fab heavy chain polypeptide chain is linked to a C terminus of the light chain variable domain of the scFv.

Disclosed herein, in some embodiments, are isolated polypeptides or polypeptide complexes comprising a structural arrangement according to Configuration 8:

wherein the isolated polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the heavy chain variable domain of the scFv with a linking moiety that is a substrate for a tumor specific protease, and the peptide is further linked to a half-life extending molecule; and a Fab that binds to TROP2, wherein the Fab comprises a Fab light chain polypeptide and a Fab heavy chain polypeptide, wherein the Fab light chain polypeptide is linked to a C terminus of the light chain variable domain of the scFv.

Disclosed herein, in some embodiments, are isolated polypeptides or polypeptide complexes comprising a structural arrangement according to Configuration 9:

wherein the isolated polypeptide or polypeptide complex comprises a Fab that binds to TROP2, the Fab comprising a Fab light chain polypeptide and a Fab heavy chain polypeptide, wherein the Fab is linked to a peptide (P₁) that impairs binding of the Fab to TROP2 and P₁ is linked to a N terminus of the Fab light chain polypeptide with a linking moiety (L₁) that is a substrate for a tumor specific protease, and the P₁ is further linked to a half-life extending molecule; and a single chain variable fragment (scFv) that binds to an effector cell antigen, the scFv comprising a light chain variable domain and a heavy chain variable domain, wherein the heavy chain variable domain of the scFv is linked to an N terminus of the Fab heavy chain polypeptide, wherein the scFv is linked to P₂ and L₂, wherein P₂ comprises a peptide that impairs binding of the scFv to the effector cell antigen, and L₂ comprises a linking moiety that connects the light chain variable domain of the scFv to P₂ and is a substrate for a tumor specific protease.

Disclosed herein, in some embodiments, are isolated polypeptides or polypeptide complexes comprising a structural arrangement according to Configuration 10:

wherein the isolated polypeptide or polypeptide complex comprises a Fab that binds to TROP2, the Fab comprising a Fab light chain polypeptide and a Fab heavy chain polypeptide, wherein the Fab is linked to a peptide that impairs binding of the Fab to TROP2 and the peptide is linked to a N terminus of the Fab light chain polypeptide with a linking moiety that is a substrate for a tumor specific protease, and the peptide is further linked to half-life extending molecule; and a single chain variable fragment (scFv) that binds to an effector cell antigen, the scFv comprising a light chain variable domain and a heavy chain variable domain, wherein the heavy chain variable domain of the scFv is linked to an N terminus of the Fab heavy chain polypeptide.

Disclosed herein, in some embodiments, are isolated polypeptides or polypeptide complexes comprising a structural arrangement according to Configuration 11:

wherein the isolated polypeptide or polypeptide complex comprises a Fab that binds to TROP2, the Fab comprising a Fab light chain polypeptide and a Fab heavy chain polypeptide, wherein the Fab is linked to a peptide (P₁) that impairs binding of the Fab to TROP2 and P₁ is linked to a N terminus of the Fab heavy chain polypeptide with a linking moiety (L₁) that is a substrate for a tumor specific protease, and P₁ is further linked to a half-life extending molecule; and a single chain variable fragment (scFv) that binds to an effector cell antigen, the scFv comprising a light chain variable domain and a heavy chain variable domain, wherein the heavy chain variable domain of the scFv is linked to an N terminus of the Fab light chain polypeptide, wherein the scFv further is linked to P₂ and L₂, wherein P₂ comprises a peptide that impairs binding of the scFv to the effector cell antigen, and L₂ comprises a linking moiety that connects the light chain variable domain of the scFv to P₂ and is a substrate for a tumor specific protease.

Disclosed herein, in some embodiments, are isolated polypeptides or polypeptide complexes comprising a structural arrangement according to Configuration 12:

wherein the isolated polypeptide or polypeptide complex comprises a Fab that binds to TROP2, the Fab comprising a Fab light chain polypeptide and a Fab heavy chain polypeptide, wherein the Fab is linked to a peptide that impairs binding of the Fab to TROP2 and the peptide is linked to a N terminus of the Fab heavy chain polypeptide with a linking moiety that is a substrate for a tumor specific protease, and the peptide is further linked to a half-life extending molecule; and a single chain variable fragment (scFv) that binds to an effector cell antigen, the scFv comprising a light chain variable domain and a heavy chain variable domain, wherein the heavy chain variable domain of the scFv is linked to an N terminus of the Fab light chain polypeptide.

Disclosed herein, in some embodiments, are isolated polypeptides or polypeptide complexes comprising a structural arrangement according to Configuration 13:

wherein the isolated polypeptide or polypeptide complex comprises a Fab that binds to TROP2, the Fab comprising a Fab light chain polypeptide and a Fab heavy chain polypeptide, wherein the Fab is linked to a peptide (P₁) that impairs binding of the Fab to TROP2 and P₁ is linked to a N terminus of the Fab light chain polypeptide with a linking moiety (L₁) that is a substrate for a tumor specific protease, and P₁ is further linked to a half-life extending molecule; and a single chain variable fragment (scFv) that binds to an effector cell antigen, the scFv comprising a light chain variable domain and a heavy chain variable domain, wherein the light chain variable domain of the scFv is linked to an N terminus of the Fab heavy chain polypeptide, wherein the scFv is linked to P₂ and L₂, wherein P₂ comprises a peptide that impairs binding of the scFv to the effector cell antigen, and L₂ comprises a linking moiety that connects the heavy chain variable domain of the scFv to P₂ and is a substrate for a tumor specific protease.

Disclosed herein, in some embodiments, are isolated polypeptides or polypeptide complexes comprising a structural arrangement according to Configuration 14:

wherein the isolated polypeptide or polypeptide complex comprises a Fab that binds to TROP2, the Fab comprising a Fab light chain polypeptide and a Fab heavy chain polypeptide, wherein the Fab is linked to a peptide that impairs binding of the Fab to TROP2 and the peptide is linked to a N terminus of the Fab light chain polypeptide with a linking moiety that is a substrate for a tumor specific protease, and the peptide is further linked to a half-life extending molecule; and a single chain variable fragment (scFv) that binds to an effector cell antigen, the scFv comprising a light chain variable domain and a heavy chain variable domain, wherein the light chain variable domain of the scFv is linked to an N terminus of the Fab heavy chain polypeptide.

Disclosed herein, in some embodiments, are isolated polypeptides or polypeptide complexes comprising a structural arrangement according to Configuration 15:

wherein the isolated polypeptide or polypeptide complex comprises a Fab that binds to TROP2, the Fab comprising a Fab light chain polypeptide and a Fab heavy chain polypeptide, wherein the Fab is linked to a (P₁) that impairs binding of the Fab to TROP2 and P₁ is linked to a N terminus of the Fab heavy chain polypeptide with a linking moiety (L₁) that is a substrate for a tumor specific protease, and P₁ is further linked to a half-life extending molecule; and a single chain variable fragment (scFv) that binds to an effector cell antigen, the scFv comprising a light chain variable domain and a heavy chain variable domain, wherein the light chain variable domain of the scFv is linked to an N terminus of the Fab light chain polypeptide, wherein the scFv is linked to P₂ and L₂, wherein P₂ comprises a peptide that impairs binding of the scFv to the effector cell antigen, and L₂ comprises a linking moiety that connects the heavy chain variable domain of the scFv to P₂ and is a substrate for a tumor specific protease.

Disclosed herein, in some embodiments, are isolated polypeptides or polypeptide complexes comprising a structural arrangement according to Configuration 16:

wherein the isolated polypeptide or polypeptide complex comprises a Fab that binds to TROP2, the Fab comprising a Fab light chain polypeptide and a Fab heavy chain polypeptide, wherein the Fab is linked to a peptide that impairs binding of the Fab to TROP2 and the peptide is linked to a N terminus of the Fab heavy chain polypeptide with a linking moiety that is a substrate for a tumor specific protease, and the peptide is further linked to a half-life extending molecule; and a single chain variable fragment (scFv) that binds to an effector cell antigen, the scFv comprising a light chain variable domain and a heavy chain variable domain, wherein the light chain variable domain of the scFv is linked to an N terminus of the Fab light chain polypeptide.

### Polynucleotides Encoding Polypeptides or Polypeptide Complexes

Disclosed herein, in some embodiments, are isolated recombinant nucleic acid molecules encoding polypeptides or polypeptide complexes as disclosed herein. In some embodiments, the polypeptides or polypeptide complexes comprise an antibody or an antibody fragment. In some embodiments, the polypeptides or polypeptide complexes comprise a Fab and a single chain variable fragment (scFv).

Disclosed herein, in some embodiments, are isolated recombinant nucleic acid molecules encoding polypeptides or polypeptide complexes according to Formula I:

A₂-A₁-L₁-P₁-H₁ (Formula I)

wherein: A₁ comprises a first antigen recognizing molecule that binds to an effector cell antigen; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to TROP2.

Disclosed herein, in some embodiments, are isolated recombinant nucleic acid molecules encoding polypeptides or polypeptide complexes according to Formula I:

A₂-A₁-L₁-P₁-H₁ (Formula I)

wherein: A₁ is a first antigen recognizing molecule that binds to an effector cell antigen; P₁ is a peptide that binds to A₁; L₁ is a linking moiety that connects A₁ to P₁ and is a substrate for a tumor specific protease; H₁ is a half-life extending molecule; and A₂ is a second antigen recognizing molecule that binds to TROP2.

Disclosed herein, in some embodiments, are isolated recombinant nucleic acid molecules encoding polypeptides or polypeptide complexes comprising Formula I:

A₂-A₁-L₁-P₁-H₁ (Formula I)

wherein: A₁ comprises a first antigen recognizing molecule that binds to an effector cell antigen; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to TROP2.

Disclosed herein, in some embodiments, are isolated recombinant nucleic acid molecules encoding polypeptides or polypeptide complexes comprising Formula I:

A₂-A₁-L₁-P₁-H₁ (Formula I)

wherein: A₁ is a first antigen recognizing molecule that binds to an effector cell antigen; P₁ is a peptide that binds to A₁; L₁ is a linking moiety that connects A₁ to P₁ and is a substrate for a tumor specific protease; H₁ is a half-life extending molecule; and A₂ is a second antigen recognizing molecule that binds to TROP2.

Disclosed herein, in some embodiments, are isolated recombinant nucleic acid molecules encoding polypeptides or polypeptide complexes according to Formula Ia:

P₂-L₂-A₂-A₁-L₁-P₁-H₁ (Formula Ia).

Disclosed herein, in some embodiments, are isolated recombinant nucleic acid molecules encoding polypeptides or polypeptide complexes according to Formula II:

L₁ₐ-P₁ₐ-H₁ₐ (Formula II)

wherein: L₁ₐ comprises a tumor specific protease-cleaved linking moiety that when uncleaved connects P₁ₐ to a first antigen recognizing molecule that binds to an effector cell antigen and the first antigen recognizing molecule is connected to a second antigen recognizing molecule that binds to TROP2; P₁ₐ comprises a peptide that binds to the first antigen recognizing molecule when L₁ₐ is uncleaved; and H₁ₐ comprises a half-life extending molecule.

Disclosed herein, in some embodiments, are isolated recombinant nucleic acid molecules encoding polypeptides or polypeptide complexes comprising Formula II:

L₁ₐ-P₁ₐ-H₁ₐ (Formula II)

wherein: L₁ₐ comprises a tumor specific protease-cleaved linking moiety that when uncleaved connects P₁ₐ to a first antigen recognizing molecule that binds to an effector cell antigen and the first antigen recognizing molecule is connected to a second antigen recognizing molecule that binds to TROP2; P₁ₐ comprises a peptide that binds to the first antigen recognizing molecule when L₁ₐ is uncleaved; and H₁ₐ comprises a half-life extending molecule.

Disclosed herein, in some embodiments, are isolated recombinant nucleic acid molecules encoding polypeptides or polypeptide complexes according to Formula II:

L₁ₐ-P₁ₐ-H₁ₐ (Formula II)

wherein: L₁ₐ is a tumor specific protease-cleaved linking moiety that when uncleaved connects P₁ₐ to a first antigen recognizing molecule that binds to an effector cell antigen and the first antigen recognizing molecule is connected to a second antigen recognizing molecule that binds to TROP2; P₁ₐ is a peptide that binds to the first antigen recognizing molecule when L₁ₐ is uncleaved; and H₁ₐ is a half-life extending molecule.

Disclosed herein, in some embodiments, are isolated recombinant nucleic acid molecules encoding polypeptides or polypeptide complexes comprising Formula II:

L₁ₐ-P₁ₐ-H₁ₐ (Formula II)

wherein: L₁ₐ is a tumor specific protease-cleaved linking moiety that when uncleaved connects P₁ₐ to a first antigen recognizing molecule that binds to an effector cell antigen and the first antigen recognizing molecule is connected to a second antigen recognizing molecule that binds to TROP2; P₁ₐ is a peptide that binds to the first antigen recognizing molecule when L₁ₐ is uncleaved; and H₁ₐ is a half-life extending molecule.

Disclosed herein, in some embodiments, are isolated nucleic acid molecules encoding polypeptides or polypeptide complexes comprising a structural arrangement according to Configuration 1:

wherein the polypeptide complex comprises a single chain variable fragment (scFv) comprising a light chain variable domain and a heavy chain variable domain, wherein the scFv further comprises a peptide that impairs binding of the scFv to an effector cell antigen and the peptide is linked to a N-terminus of the heavy chain variable domain of the scFv with a linking moiety that is a substrate for a tumor specific protease, and the peptide further comprises a half-life extending molecule; and a Fab that binds to TROP2, wherein the Fab comprises a Fab light chain polypeptide and a Fab heavy chain polypeptide, wherein the Fab heavy chain polypeptide is linked to a C terminus of the light chain variable domain of the scFv, and wherein the Fab further comprises P₂ and L₂, wherein P₂ comprises a peptide that impairs binding to TROP2; and L₂ comprises a linking moiety that connects the Fab light chain polypeptide to P₂ and is a substrate for a tumor specific protease.

Disclosed herein, in some embodiments, are isolated nucleic acid molecules encoding polypeptides or polypeptide complexes comprising a structural arrangement according to Configuration 2:

wherein the polypeptide complex comprises a single chain variable fragment (scFv) comprising a light chain variable domain and a heavy chain variable domain, wherein the scFv further comprises a peptide that impairs binding of the scFv to an effector cell antigen and the peptide is linked to a N-terminus of the heavy chain variable domain of the scFv with a linking moiety that is a substrate for a tumor specific protease, and the peptide further comprises a half-life extending molecule; and a Fab that binds to TROP2, wherein the Fab comprises a Fab light chain polypeptide and a Fab heavy chain polypeptide, wherein the Fab light chain polypeptide is linked to a C terminus of the light chain variable domain of the scFv, and wherein the Fab further comprises P₂ and L₂, wherein P₂ comprises a peptide that impairs binding to TROP2; and L₂ comprises a linking moiety that connects the Fab heavy chain polypeptide to P₂ and is a substrate for a tumor specific protease.

### Pharmaceutical Compositions

Disclosed herein, in some embodiments, are pharmaceutical compositions comprising: (a) the isolated polypeptides or polypeptide complexes as disclosed herein; and (b) a pharmaceutically acceptable excipient.

In some embodiments, the pharmaceutical composition comprises (a) isolated polypeptides or polypeptide complexes according to Formula I:

A₂-A₁-L₁-P₁-H₁ (Formula I)

wherein: A₁ comprises a first antigen recognizing molecule that binds to an effector cell antigen; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to TROP2; and (b) a pharmaceutically acceptable excipient.

In some embodiments, the pharmaceutical composition comprises (a) isolated polypeptides or polypeptide complexes according to Formula I:

A₂-A₁-L₁-P₁-H₁ (Formula I)

wherein: A₁ is a first antigen recognizing molecule that binds to an effector cell antigen; P₁ is a peptide that binds to A₁; L₁ is a linking moiety that connects A₁ to P₁ and is a substrate for a tumor specific protease; H₁ is a half-life extending molecule; and A₂ is a second antigen recognizing molecule that binds to TROP2; and (b) a pharmaceutically acceptable excipient.

In some embodiments, the pharmaceutical composition comprises (a) isolated polypeptides or polypeptide complexes comprising Formula I:

A₂-A₁-L₁-P₁-H₁ (Formula I)

wherein: A₁ comprises a first antigen recognizing molecule that binds to an effector cell antigen; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to TROP2; and (b) a pharmaceutically acceptable excipient.

In some embodiments, the pharmaceutical composition comprises (a) isolated polypeptides or polypeptide complexes comprising Formula I:

A₂-A₁-L₁-P₁-H₁ (Formula I)

wherein: A₁ is a first antigen recognizing molecule that binds to an effector cell antigen; P₁ is a peptide that binds to A₁; L₁ is a linking moiety that connects A₁ to P₁ and is a substrate for a tumor specific protease; H₁ is a half-life extending molecule; and A₂ is a second antigen recognizing molecule that binds to TROP2; and (b) a pharmaceutically acceptable excipient.

In some embodiments, the pharmaceutical composition comprises (a) isolated polypeptides or polypeptide complexes according to Formula Ia:

P₂-L₂-A₂-A₁-L₁-P₁-H₁ (Formula Ia);

and (b) a pharmaceutically acceptable excipient.

In some embodiments, the pharmaceutical composition comprises (a) isolated polypeptides or polypeptide complexes according to Formula II:

L₁ₐ-P₁ₐ-H₁ₐ (Formula II)

wherein: L₁ₐ comprises a tumor specific protease-cleaved linking moiety that when uncleaved connects P₁ₐ to a first antigen recognizing molecule that binds to an effector cell antigen and the first antigen recognizing molecule is connected to a second antigen recognizing molecule that binds to TROP2; P₁ₐ comprises a peptide that binds to the first antigen recognizing molecule when L₁ₐ is uncleaved; and H₁ₐ comprises a half-life extending molecule; and (b) a pharmaceutically acceptable excipient.

In some embodiments, the pharmaceutical composition comprises (a) isolated polypeptides or polypeptide complexes comprising Formula II:

L₁ₐ-P₁ₐ-H₁ₐ (Formula II)

wherein: L₁ₐ comprises a tumor specific protease-cleaved linking moiety that when uncleaved connects P₁ₐ to a first antigen recognizing molecule that binds to an effector cell antigen and the first antigen recognizing molecule is connected to a second antigen recognizing molecule that binds to TROP2; P₁ₐ comprises a peptide that binds to the first antigen recognizing molecule when L₁ₐ is uncleaved; and H₁ₐ comprises a half-life extending molecule; and (b) a pharmaceutically acceptable excipient.

In some embodiments, the pharmaceutical composition comprises (a) isolated polypeptides or polypeptide complexes according to Formula II:

L₁ₐ-P₁ₐ-H₁ₐ (Formula II)

wherein: L₁ₐ is a tumor specific protease-cleaved linking moiety that when uncleaved connects P₁ₐ to a first antigen recognizing molecule that binds to an effector cell antigen and the first antigen recognizing molecule is connected to a second antigen recognizing molecule that binds to TROP2; P₁ₐ is a peptide that binds to the first antigen recognizing molecule when L₁ₐ is uncleaved; and H₁ₐ is a half-life extending molecule; and (b) a pharmaceutically acceptable excipient.

In some embodiments, the pharmaceutical composition comprises (a) isolated polypeptides or polypeptide complexes comprising Formula II:

L₁ₐ-P₁ₐ-H₁ₐ (Formula II)

wherein: L₁ₐ is a tumor specific protease-cleaved linking moiety that when uncleaved connects P₁ₐ to a first antigen recognizing molecule that binds to an effector cell antigen and the first antigen recognizing molecule is connected to a second antigen recognizing molecule that binds to TROP2; P₁ₐ is a peptide that binds to the first antigen recognizing molecule when L₁ₐ is uncleaved; and H₁ₐ is a half-life extending molecule; and (b) a pharmaceutically acceptable excipient.

Disclosed herein, in some embodiments, the pharmaceutical composition comprises (a) isolated polypeptides or polypeptide complexes comprising a structural arrangement according to Configuration 1:

wherein the polypeptide complex comprises a single chain variable fragment (scFv) comprising a light chain variable domain and a heavy chain variable domain, wherein the scFv further comprises a peptide that impairs binding of the scFv to an effector cell antigen and the peptide is linked to a N-terminus of the heavy chain variable domain of the scFv with a linking moiety that is a substrate for a tumor specific protease, and the peptide further comprises a half-life extending molecule; and a Fab that binds to TROP2, wherein the Fab comprises a Fab light chain polypeptide and a Fab heavy chain polypeptide, wherein the Fab heavy chain polypeptide is linked to a C terminus of the light chain variable domain of the scFv, and wherein the Fab further comprises P₂ and L₂, wherein P₂ comprises a peptide that impairs binding to TROP2; and L₂ comprises a linking moiety that connects the Fab light chain polypeptide to P₂ and is a substrate for a tumor specific protease; and (b) a pharmaceutically acceptable excipient.

Disclosed herein, in some embodiments, the pharmaceutical composition comprises (a) isolated polypeptides or polypeptide complexes comprising a structural arrangement according to Configuration 2:

wherein the polypeptide complex comprises a single chain variable fragment (scFv) comprising a light chain variable domain and a heavy chain variable domain, wherein the scFv further comprises a peptide that impairs binding of the scFv to an effector cell antigen and the peptide is linked to a N-terminus of the heavy chain variable domain of the scFv with a linking moiety that is a substrate for a tumor specific protease, and the peptide further comprises a half-life extending molecule; and a Fab that binds to TROP2, wherein the Fab comprises a Fab light chain polypeptide and a Fab heavy chain polypeptide, wherein the Fab light chain polypeptide is linked to a C terminus of the light chain variable domain of the scFv, and wherein the Fab further comprises P₂ and L₂, wherein P₂ comprises a peptide that impairs binding to TROP2; and L₂ comprises a linking moiety that connects the Fab heavy chain polypeptide to P₂ and is a substrate for a tumor specific protease; and (b) a pharmaceutically acceptable excipient.

In some embodiments, the isolated polypeptide or polypeptide complex further comprises a detectable label, a therapeutic agent, or a pharmacokinetic modifying moiety. In some embodiments, the detectable label comprises a fluorescent label, a radiolabel, an enzyme, a nucleic acid probe, or a contrast agent.

For administration to a subject, the isolated polypeptide or polypeptide complex as disclosed herein, may be provided in a pharmaceutical composition together with one or more pharmaceutically acceptable carriers or excipients. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

The pharmaceutical composition may be in any suitable form, (depending upon the desired method of administration). It may be provided in unit dosage form, may be provided in a sealed container and may be provided as part of a kit. Such a kit may include instructions for use. It may include a plurality of said unit dosage forms.

The pharmaceutical composition may be adapted for administration by any appropriate route, including a parenteral (e.g., subcutaneous, intramuscular, or intravenous) route. Such compositions may be prepared by any method known in the art of pharmacy, for example by mixing the active ingredient with the carrier(s) or excipient(s) under sterile conditions.

Dosages of the substances of the present disclosure can vary between wide limits, depending upon the disease or disorder to be treated, the age and condition of the individual to be treated, etc. and a physician will ultimately determine appropriate dosages to be used.

Peptides that Impair Binding of Anti-TROP2 Binding Domains to TROP2

Disclosed herein are isolated polypeptide or polypeptide complexes comprising an anti-TROP2 binding domain that is linked to a peptide that impairs binding of the anti-TROP2 binding to TROP2 wherein the peptide comprises an amino acid sequence according to X₁-X₂-X₃-X₄-C-X₆-X₇-X₈-X₉-X₁₀-C-X₁₂-X₁₃-X₁₄ and X₁ is selected from N, D, S, Y, A, F, H, T, L, and V; X₂ is selected from S, T, D, A, H, V, Y, N, F, I, and L; X₃ is selected from L, I, and V; X₄ is selected from F, L, V, M, W, I, Y, and H; X₆ is selected from V, F, L, I, and W; X₇ is selected from K, R, Q, N, H, and M; X₈ is selected from N and K; X₉ is selected from L, V, and I; X₁₀ is selected from Y, W, F, Q, and L; X₁₂ is selected from W and V; X₁₃ is selected from I, N, H, T, V, Y, and D; X₁₄ is selected from D, V, A, S, I, T, N, Y, H, and P; or the peptide comprises an amino acid according to J₁-J₂-J₃-C-J₈-J₆-J₇-J₈-W-J₁₀-J₁₁-C-J₁₃-J₁₄ and J₁ is selected from V, I, L, P, E, F, and M; J₂ is selected from D and N; J₃ is selected from F and W; J₈ is selected from A, E, S, R, K, Y, L, Q, G, M, F, T, W, and D; J₆ is selected from L, M, I, V, F, T, R, and S; J₇ is selected from Y, F, and N; J₈ is selected from N, R, D, H, K, Q, S, G, A, E, and M; J₁₀ is selected from P and R; J₁₁ is selected from V and I; J₁₃ is selected from D, G, N, R, S, Q, Y, T, A, E, L, V, K, M, I, H, F, and W; J₁₄ is selected from T, S, Q, L, D, N, A, E, K, M, V, R, I, H, P, V, and W; or the peptide comprises an amino acid sequence according to B₁-B₂-B₃-C-B₅-B₆-B₇-B₈-W-B₁₀-B₁₁-C-B₁₃-B₁₄ and B₁ is selected from V, I, L, P, E, F, and M; B₂ is selected from D and N; B₃ is selected from F and W; B₈ is selected from A, E, S, R, K, Y, L, Q, G, M, F, T, W, and D; B₆ is selected from L, M, I, V, F, T, R, and S; B₇ is selected from Y, F, and N; B₈ is selected from N, R, D, H, K, Q, S, G, A, E, and M; B₁₀ is selected from P and R; B₁₁ is selected from V and I; B₁₃ is selected from D, G, N, R, S, Q, Y, T, A, E, L, V, K, M, I, H, F, and W; and B₁₄ is selected from T, S, Q, L, D, N, A, E, K, M, V, R, I, H, P, V, G, and W. In some embodiments, X₁ is selected from N, D, S, Y, A, F, and T; X₂ is selected from S, T, D, A, H, V, Y, and N; X₃ is L; X₄ is selected from F, L, V, M, and W; X₆ is selected from V, F, and L; X₇ is selected from K, R, Q, and N; X₈ is selected from N and K; X₉ is selected from L, V, and I; X₁₀ is selected from Y, W, and F; X₁₂ is W; X₁₃ is selected from I, N, H, and T; and X₁₄ is selected from D, V, A, S, I, T, and N. In some embodiments, X₁ is selected from N, D, S, and Y; X₂ is selected from S, T, and D; X₃ is L; X₄ is selected from F, L, and V; X₆ is selected from V and F; X₇ is selected from K, R, and Q; X₈ is N; X₉ is selected from L and V; X₁₀ is selected from Y and W; X₁₂ is W; X₁₃ is selected from I, N, and H; and X₁₄ is selected from D, V, A, and S. In some embodiments, J₁ is selected from V, I, and L; J₂ is D; J₃ is F; J₈ is selected from A, E, S, R, K, Y, and L; J₆ is selected from L, M, and I; J₇ is Y; J₈ is selected from N, R, D, H, K, Q, S, and G; J₁₀ is P; J₁₁ is selected from V and I; J₁₃ is selected from D, G, N, R, S, Q, Y, T, and A; and J₁₄ is selected from T, S, Q, L, D, N, A, and E. In some embodiments, J₁ is selected from V, I, and L; J₂ is D; J₃ is F; J₈ is selected from A, E, S, and R; J₆ is selected from L, M, and I; J₇ is Y; J₈ is selected from N, R, and D; J₁₀ is P; J₁₁ is selected from V and I; J₁₃ is selected from D, G, N, R, S, and Q; J₁₄ is selected from T, S, Q, and L. In some embodiments, B₁ is selected from V, I, and L; B₂ is D; B₃ is F; B₈ is selected from A, E, S, R, K, Y, M, G, and L; B₆ is selected from L, M, and I; B₇ is Y; B₈ is selected from N, R, D, H, K, Q, S, and G; B₁₀ is P; B₁₁ is selected from V and I; B₁₃ is selected from D, G, N, R, S, Q, Y, T, H, and A; B₁₄ is selected from T, S, Q, L, D, N, A, G, and E. In some embodiments, B₁ is selected from V, I, and L; B₂ is D; B₃ is F; B₈ is selected from A, E, S, K, M, G, and R; B₆ is selected from L, M, and I; B₇ is Y; B₈ is selected from N, R, S, H, and D; B₁₀ is P; B₁₁ is selected from V and I; B₁₃ is selected from D, G, N, R, S, H, A, Y, and Q; and B₁₄ is selected from T, S, Q, G, and L.

In some embodiments, the peptide comprises the amino acid sequences according to SEQ ID NOs: 133-145.

In some embodiments, the peptide comprises the amino acid sequences according to SEQ ID NOs: 146-158.

In some embodiments, the peptide comprises an amino acid sequence according to any of the sequences of Table 27.

In some embodiments, the peptide comprises the amino acid sequences according to SEQ ID NOs: 159-178.

In some embodiments, the peptide comprises an amino acid sequences according to any of the sequences of Table 29.

In some embodiments, the peptide comprises the amino acid sequences according to SEQ ID NOs: 179-201.

In some embodiments, the peptide comprises the amino acid sequence according to SEQ ID NO: 24 or a peptide sequence that has 1, 2, or 3, amino acid substitutions, additions, or deletions relative to the amino acid sequence of SEQ ID NO: 24.

In some embodiments, the peptide comprises the amino acid sequence according to SEQ ID NO: 181 or a peptide sequence that has 1, 2, or 3, amino acid substitutions, additions, or deletions relative to the amino acid sequence of SEQ ID NO: 181.

In some embodiments, the peptide comprises the amino acid sequence according to SEQ ID NO: 186 or a peptide sequence that has 1, 2, or 3, amino acid substitutions, additions, or deletions relative to the amino acid sequence of SEQ ID NO: 186.

In some embodiments, the peptide comprises the amino acid sequence according to SEQ ID NO: 24.

In some embodiments, the peptide comprises the amino acid sequence according to SEQ ID NO: 181.

In some embodiments, the peptide comprises the amino acid sequence according to SEQ ID NO: 186.

In some embodiments, the anti-TROP2 binding domain comprises an antibody or an antibody fragment.

In some embodiments, the antibody or antibody fragment comprises a single chain variable fragment, a single domain antibody, Fab, or Fab′.

In some embodiments, the anti-TROP2 binding domain comprises heavy chain complementarity determining regions HC-CDR1, HC-CDR2, and HC-CDR3, wherein the HC-CDR1, the HC-CDR2, and the HC-CDR3 comprise: HC-CDR1: SEQ ID NO: 15, HC-CDR2: SEQ ID NO: 16, and HC-CDR3: SEQ ID NO: 17; and the anti-TROP2 binding domain comprises light chain complementarity determining regions CDRs: LC-CDR1, LC-CDR2, and LC-CDR3, wherein the LC-CDR1, the LC-CDR2, and the LC-CDR3 of the Fab comprise LC-CDR1: SEQ ID NO: 18, LC-CDR2: SEQ ID NO:19, and LC-CDR3: SEQ ID NO: 20.

In some embodiments, the antibody or antibody fragment comprises the Fab. In some embodiments, the anti-TROP2 binding domain comprises amino acid sequences according to SEQ ID NOs: 21-22. In some embodiments, the isolated polypeptide or polypeptide complex further comprises a half-life extending molecule (H₁). In some embodiments, the half-life extending molecule is linked to the peptide. In some embodiments, H₁ comprises a polymer. In some embodiments, the polymer is polyethylene glycol (PEG). In some embodiments, H₁ comprises albumin. In some embodiments, H₁ comprises an Fc domain. In some embodiments, the albumin is serum albumin. In some embodiments, the albumin is human serum albumin. In some embodiments, H₁ comprises a polypeptide, a ligand, or a small molecule. In some embodiments, the polypeptide, the ligand or the small molecule binds serum protein or a fragment thereof, a circulating immunoglobulin or a fragment thereof, or CD35/CR1. In some embodiments, the serum protein comprises a thyroxine-binding protein, a transthyretin, a 1-acid glycoprotein, a transferrin, transferrin receptor or a transferrin-binding portion thereof, a fibrinogen, or an albumin. In some embodiments, the circulating immunoglobulin molecule comprises IgG1, IgG2, IgG3, IgG4, slgA, IgM or IgD. In some embodiments, the serum protein is albumin. In some embodiments, the polypeptide is an antibody. In some embodiments, the antibody comprises a single domain antibody, a single chain variable fragment, or a Fab. In some embodiments, the single domain antibody comprises a single domain antibody that binds to albumin. In some embodiments, the single domain antibody is a human or humanized antibody. In some embodiments, the single domain antibody is 645gH1gL1. In some embodiments, the single domain antibody is 645dsgH5gL4. In some embodiments, the single domain antibody is 23-13-A01-sc02. In some embodiments, the single domain antibody is A10m3 or a fragment thereof. In some embodiments, the single domain antibody is DOM7r-31. In some embodiments, the single domain antibody is DOM7h-11-15. In some embodiments, the single domain antibody is Alb-1, Alb-8, or Alb-23. In some embodiments, the single domain antibody is 10E. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 66, HC-CDR2: SEQ ID NO: 67, and HC-CDR3: SEQ ID NO: 68. In some embodiments, the single domain antibody comprises an amino acid sequence according to SEQ ID NO: 69. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 70, HC-CDR2: SEQ ID NO: 71, and HC-CDR3: SEQ ID NO: 72. In some embodiments, the single domain antibody comprises an amino acid sequence according to SEQ ID NO: 73. In some embodiments, the single domain antibody is SA21. In some embodiments, the isolated polypeptide or polypeptide complex comprises a modified amino acid, a non-natural amino acid, a modified non-natural amino acid, or a combination thereof. In some embodiments, the modified amino acid or modified non-natural amino acid comprises a post-translational modification. In some embodiments, H₁ comprises a linking moiety (L₃) that connects H₁ to the peptide. In some embodiments, L₃ is a peptide sequence having at least 5 to no more than 50 amino acids. In some embodiments, L₃ is a peptide sequence having at least 10 to no more than 30 amino acids. In some embodiments, L₃ is a peptide sequence having at least 10 amino acids. In some embodiments, L₃ is a peptide sequence having at least 18 amino acids. In some embodiments, L₃ is a peptide sequence having at least 26 amino acids. In some embodiments, L₃ has a formula selected from the group consisting of (G₂S)ₙ, (GS)ₙ, (GSGGS)ₙ (SEQ ID NO: 62), (GGGS)ₙ (SEQ ID NO: 63), (GGGGS)ₙ (SEQ ID NO: 64), and (GSSGGS)ₙ (SEQ ID NO: 65), wherein n is an integer of at least 1. In some embodiments, L₃ comprises an amino acid sequence according to SEQ ID NO: 30.

### Methods of Treatment

In some embodiments, are methods of treating cancer in a subject need in need thereof comprising administering to the subject an isolated polypeptide or polypeptide complex as described herein. In some embodiments, the cancer has cells that express TROP2. In some instances, the cancer is a solid tumor cancer. In some embodiments, the cancer is lung, breast (e.g. HER2+; ER/PR+; TNBC), cervical, ovarian, colorectal, pancreatic or gastric.

In some embodiments, are methods of treating triple-negative breast cancer (TNBC), urothelial cancer (UC), non-small cell lung cancer (NSCLC), small cell lung cancer (SCLC), gastric cancer, esophageal cancer, head and neck cancer, prostate cancer, or endometrial cancer in a subject need in need thereof comprising administering to the subject an isolated polypeptide or polypeptide complex as described herein. In some embodiments, are methods of treating breast cancer, lung cancer, urothelial cancer, endometrial cancer, ovarian cancer, prostate cancer, pancreatic cancer, gastric cancer, colon cancer, head and neck cancer, and glioma in a subject need in need thereof comprising administering to the subject an isolated polypeptide or polypeptide complex as described herein.

Described herein, in some embodiments, are polypeptides or polypeptide complexes, wherein the polypeptides or polypeptide complexes comprise a long half-life. In some instances, the half-life of the polypeptides or polypeptide complexes is at least or about 12 hours, 24 hours 36 hours, 48 hours, 60 hours, 72 hours, 84 hours, 96 hours, 100 hours, 108 hours, 119 hours, 120 hours, 140 hours, 160 hours, 180 hours, 200 hours, or more than 200 hours. In some instances, the half-life of the polypeptides or polypeptide complexes is in a range of about 12 hours to about 300 hours, about 20 hours to about 280 hours, about 40 hours to about 240 hours, about 60 hours to about 200 hours, or about 80 hours to about 140 hours.

Described herein, in some embodiments, are isolated polypeptide or polypeptide complexes administered as once weekly. In some embodiments, the isolated polypeptide or polypeptide complexes are administered once weekly by intravenous, intramuscular, intralesional, topical, subcutaneous, infusion, or oral. In some embodiments, the isolated polypeptide or polypeptide complexes are administered once weekly by bolus injection. In some embodiments, the isolated polypeptide or polypeptide complexes are administered once weekly by continuous infusion. In some embodiments, the isolated polypeptide or polypeptide complex is administered to the subject once a week as a continuous infusion over a period of no more than 60 minutes. In some embodiments, the isolated polypeptide or polypeptide complex is administered to the subject once a week as a continuous intravenous infusion over a period of no more than 30 minutes. In some embodiments, the isolated polypeptide or polypeptide complex is administered to the subject once a week as a continuous intravenous infusion over a period of at least 10 minutes.

In some embodiments, the isolated polypeptide or polypeptide complex is administered to the subject once a week and the isolated polypeptide or polypeptide complex has a half-life of at least 30 hours. In some embodiments, the isolated polypeptide or polypeptide complex is administered to the subject once a week and the isolated polypeptide or polypeptide complex has a half-life of at least 50 hours. In some embodiments, the isolated polypeptide or polypeptide complex is administered to the subject once a week and the isolated polypeptide or polypeptide complex has a half-life of at least 60 hours. In some embodiments, the isolated polypeptide or polypeptide complex is administered to the subject once a week and the isolated polypeptide or polypeptide complex has a half-life of at least 70 hours. In some embodiments, the isolated polypeptide or polypeptide complex is administered to the subject once a week and the isolated polypeptide or polypeptide complex has a half-life of at least 80 hours. In some embodiments, the isolated polypeptide or polypeptide complex is administered to the subject once a week and the isolated polypeptide or polypeptide complex has a half-life of at least 90 hours. In some embodiments, the isolated polypeptide or polypeptide complex is administered to the subject once a week and the isolated polypeptide or polypeptide complex has a half-life of at least 100 hours. In some embodiments, the isolated polypeptide or polypeptide complex is administered to the subject once a week and the isolated polypeptide or polypeptide complex has a half-life of at least 110 hours. In some embodiments, the isolated polypeptide or polypeptide complex is administered to the subject once a week and the isolated polypeptide or polypeptide complex has a half-life of at least 115 hours.

Production of Antibodies that Bind to TROP2 and CD3

In some embodiments, polypeptides described herein (e.g., antibodies and its binding fragments) are produced using any method known in the art to be useful for the synthesis of polypeptides (e.g., antibodies), in particular, by chemical synthesis or by recombinant expression, and are preferably produced by recombinant expression techniques.

In some instances, an antibody or its binding fragment thereof is expressed recombinantly, and the nucleic acid encoding the antibody or its binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or its binding fragment thereof is optionally generated by immunizing an animal, such as a mouse, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific embodiments, the expression of the antibody is regulated by a constitutive, an inducible or a tissue, specific promoter.

In some embodiments, a variety of host-expression vector systems is utilized to express an antibody, or its binding fragment described herein. Such host-expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long-term, high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to the herpes simplex virus thymidine kinase (Wigler et al., 1977, Cell 11:223), hypoxanthine-guanine phosphoribosyltransferase (Szybalska & Szybalski, 192, Proc. Natl. Acad. Sci. USA 48:202), and adenine phosphoribosyltransferase (Lowy et al., 1980, Cell 22:817) genes are employed in tk−, hgprt− or aprt− cells, respectively. Also, antimetabolite resistance are used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May 1993, TIB TECH 11(5):155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1).

In some instances, the expression levels of an antibody are increased by vector amplification (for a review, see Bebbington and Hentschel, the use of vectors based on gene amplification for the expression of cloned genes in mammalian cells in DNA cloning, Vol. 3. (Academic Press, New York, 1987)). When a marker in the vector system expressing an antibody is amplifiable, an increase in the level of inhibitor present in culture of host cell will increase the number of copies of the marker gene. Since the amplified region is associated with the nucleotide sequence of the antibody, production of the antibody will also increase (Crouse et al., 1983, Mol. Cell Biol. 3:257).

In some instances, any method known in the art for purification of an antibody is used, for example, by chromatography (e.g., ion exchange, affinity, particularly by affinity for the specific antigen after Protein A, and sizing column chromatography), centrifugation, differential solubility, or by any other standard technique for the purification of proteins.

### Expression Vectors

In some embodiments, vectors include any suitable vectors derived from either a eukaryotic or prokaryotic sources. In some cases, vectors are obtained from bacteria (e.g. E. coli), insects, yeast (e.g. Pichia pastoris), algae, or mammalian sources. Exemplary bacterial vectors include pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Exemplary insect vectors include pFastBacl, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

In some cases, yeast vectors include Gateway® pDEST™ 14 vector, Gateway@ pDEST™ 15 vector, Gateway@ pDEST™ 17 vector, Gateway® pDEST™ 24 vector, Gateway® pYES-DEST52 vector, pBAD-DEST49 Gateway® destination vector, pAO815 Pichia vector, pFLD1 Pichi pastoris vector, pGAPZA, B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Exemplary algae vectors include pChlamy-4 vector or MCS vector.

Examples of mammalian vectors include transient expression vectors or stable expression vectors. Mammalian transient expression vectors may include pRK5, p3×FLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3×FLAG-CMV 7.1, pFLAG-CMV 20, p3×FLAG-Myc-CMV 24, pCMV-FLAG-MAT1, pCMV-FLAG-MAT2, pBICEP-CMV 3, or pBICEP-CMV 4. Mammalian stable expression vector may include pFLAG-CMV 3, p3×FLAG-CMV 9, p3×FLAG-CMV 13, pFLAG-Myc-CMV 21, p3×FLAG-Myc-CMV 25, pFLAG-CMV 4, p3×FLAG-CMV 10, p3×FLAG-CMV 14, pFLAG-Myc-CMV 22, p3×FLAG-Myc-CMV 26, pBICEP-CMV 1, or pBICEP-CMV 2.

In some instances, a cell-free system is a mixture of cytoplasmic and/or nuclear components from a cell and is used for in vitro nucleic acid synthesis. In some cases, a cell-free system utilizes either prokaryotic cell components or eukaryotic cell components. Sometimes, a nucleic acid synthesis is obtained in a cell-free system based on for example Drosophila cell, Xenopus egg, or HeLa cells. Exemplary cell-free systems include, but are not limited to, E. coli S30 Extract system, E. coli T7 S30 system, or PURExpress®.

### Host Cells

In some embodiments, a host cell includes any suitable cell such as a naturally derived cell or a genetically modified cell. In some instances, a host cell is a production host cell. In some instances, a host cell is a eukaryotic cell. In other instances, a host cell is a prokaryotic cell. In some cases, a eukaryotic cell includes fungi (e.g., yeast cells), animal cell or plant cell. In some cases, a prokaryotic cell is a bacterial cell. Examples of bacterial cell include gram-positive bacteria or gram-negative bacteria. Sometimes the gram-negative bacteria is anaerobic, rod-shaped, or both.

In some instances, gram-positive bacteria include Actinobacteria, Firmicutes or Tenericutes. In some cases, gram-negative bacteria include Aquificae, Deinococcus-Thermus, Fibrobacteres-Chlorobi/Bacteroidetes (FCB group), Fusobacteria, Gemmatimonadetes, Nitrospirae, Planctomycetes-Verrucomicrobia/Chlamydiae (PVC group), Proteobacteria, Spirochaetes or Synergistetes. Other bacteria can be Acidobacteria, Chloroflexi, Chrysiogenetes, Cyanobacteria, Deferribacteres, Dictyoglomi, Thermodesulfobacteria or Thermotogae. A bacterial cell can be Escherichia coli, Clostridium botulinum, or Coli bacilli.

Exemplary prokaryotic host cells include, but are not limited to, BL21, Mach1™, DH10B™ TOP10, DH5α, DH10Bac™, OmniMax™, MegaX™, DH12S™, INV110, TOP10F′, INVαF, TOP10/P₃, ccdB Survival, PIR1, PIR2, Stbl2™, Stbl3™, or Stbl4™.

In some instances, animal cells include a cell from a vertebrate or from an invertebrate. In some cases, an animal cell includes a cell from a marine invertebrate, fish, insects, amphibian, reptile, or mammal. In some cases, a fungus cell includes a yeast cell, such as brewer's yeast, baker's yeast, or wine yeast.

Fungi include ascomycetes such as yeast, mold, filamentous fungi, basidiomycetes, or zygomycetes. In some instances, yeast includes Ascomycota or Basidiomycota. In some cases, Ascomycota includes Saccharomycotina (true yeasts, e.g. Saccharomyces cerevisiae (baker's yeast)) or Taphrinomycotina (e.g. Schizosaccharomycetes (fission yeasts)). In some cases, Basidiomycota includes Agaricomycotina (e.g. Tremellomycetes) or Pucciniomycotina (e.g. Microbotryomycetes).

Exemplary yeast or filamentous fungi include, for example, the genus: Saccharomyces, Schizosaccharomyces, Candida, Pichia, Hansenula, Kluyveromyces, Zygosaccharomyces, Yarrowia, Trichosporon, Rhodosporidi, Aspergillus, Fusarium, or Trichoderma. Exemplary yeast or filamentous fungi include, for example, the species: Saccharomyces cerevisiae, Schizosaccharomyces pombe, Candida utilis, Candida boidini, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Pichia metanolica, Pichia angusta, Pichia pastoris, Pichia anomala, Hansenula polymorpha, Kluyveromyces lactis, Zygosaccharomyces rouxii, Yarrowia lipolytica, Trichosporon pullulans, Rhodosporidium toru-Aspergillus niger, Aspergillus nidulans, Aspergillus awamori, Aspergillus oryzae, Trichoderma reesei, Yarrowia lipolytica, Brettanomyces bruxellensis, Candida stellata, Schizosaccharomyces pombe, Torulaspora delbrueckii, Zygosaccharomyces baili, Cryptococcus neoformans, Cryptococcus gattii, or Saccharomyces boulardii.

Exemplary yeast host cells include, but are not limited to, Pichia pastoris yeast strains such as GS115, KM71H, SMD1168, SMD1168H, and X-33; and Saccharomyces cerevisiae yeast strain such as INVScl.

In some instances, additional animal cells include cells obtained from a mollusk, arthropod, annelid or sponge. In some cases, an additional animal cell is a mammalian cell, e.g., from a primate, ape, equine, bovine, porcine, canine, feline or rodent. In some cases, a rodent includes mouse, rat, hamster, gerbil, hamster, chinchilla, fancy rat, or guinea pig.

Exemplary mammalian host cells include, but are not limited to, 293A cell line, 293FT cell line, 293F cells, 293 H cells, CHO DG44 cells, CHO-S cells, CHO-K1 cells, FUT8 KO CHOK1, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line.

In some instances, a mammalian host cell is a stable cell line, or a cell line that has incorporated a genetic material of interest into its own genome and has the capability to express the product of the genetic material after many generations of cell division. In some cases, a mammalian host cell is a transient cell line, or a cell line that has not incorporated a genetic material of interest into its own genome and does not have the capability to express the product of the genetic material after many generations of cell division.

Exemplary insect host cells include, but are not limited to, Drosophila S2 cells, Sf9 cells, Sf21 cells, High Five™ cells, and expresSF+® cells.

In some instances, plant cells include a cell from algae. Exemplary insect cell lines include, but are not limited to, strains from Chlamydomonas reinhardtii 137c, or Synechococcus elongatus PPC7942.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least one active agent in the composition is a bispecific antibody comprising a first antigen-binding site that specifically binds to CD3 and a second antigen-binding site that specifically binds to TROP2 as defined herein before.

The label or package insert indicates that the composition is used for treating the condition of choice. Moreover, the article of manufacture may comprise (a) a first container with a composition contained therein, wherein the composition comprises the bispecific antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Certain Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “antibody” is used in the broadest sense and covers fully assembled antibodies, antibody fragments that can bind antigen, for example, Fab, F(ab′)2, Fv, single chain antibodies (scFv), diabodies, antibody chimeras, hybrid antibodies, bispecific antibodies, and the like.

The term “complementarity determining region” or “CDR” is a segment of the variable region of an antibody that is complementary in structure to the epitope to which the antibody binds and is more variable than the rest of the variable region. Accordingly, a CDR is sometimes referred to as hypervariable region. A variable region comprises three CDRs. CDR peptides can be obtained by constructing genes encoding the CDR of an antibody of interest. Such genes are prepared, for example, by using the polymerase chain reaction to synthesize the variable region from RNA of antibody-producing cells. See, for example, Larrick et al., Methods: A Companion to Methods in Enzymology 2: 106 (1991); Courtenay-Luck, “Genetic Manipulation of Monoclonal Antibodies,” in Monoclonal Antibodies: Production, Engineering and Clinical Application, Ritter et al. (eds.), pages 166-179 (Cambridge University Press 1995); and Ward et al., “Genetic Manipulation and Expression of Antibodies,” in Monoclonal Antibodies: Principles and Applications, Birch et al., (eds.), pages 137-185 (Wiley-Liss, Inc. 1995).

The term “Fab” refers to a protein that contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

A “single-chain variable fragment (scFv)” is a fusion protein of the variable regions of the heavy (VH) and light chains (VL) of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### EMBODIMENTS

Embodiment 1 comprises an isolated polypeptide or polypeptide complex according to Formula I: A₂-A₁-L₁-P₁-H₁ wherein: A₁ comprises a first antigen recognizing molecule that binds to an effector cell antigen; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to TROP2.

Embodiment 2 comprises an isolated polypeptide or polypeptide complex of embodiment 1, wherein the first antigen recognizing molecule comprises an antibody or antibody fragment.

Embodiment 3 comprises an isolated polypeptide or polypeptide complex of any one of embodiments 1-2, wherein the first antigen recognizing molecule comprises an antibody or antibody fragment that is human or humanized.

Embodiment 4 comprises an isolated polypeptide or polypeptide complex of any one of embodiments 1-3, wherein L₁ is bound to N-terminus of the first antigen recognizing molecule.

Embodiment 5 comprises an isolated polypeptide or polypeptide complex of any one of embodiments 1-4, wherein A₂ is bound to C-terminus of the first antigen recognizing molecule.

Embodiment 6 comprises an isolated polypeptide or polypeptide complex of any one of embodiments 1-3, wherein L₁ is bound to C-terminus of the first antigen recognizing molecule.

Embodiment 7 comprises an isolated polypeptide or polypeptide complex of any one of embodiments 1-3 and 6, wherein A₂ is bound to N-terminus of the first antigen recognizing molecule.

Embodiment 8 comprises an isolated polypeptide or polypeptide complex of embodiment 2, wherein the antibody or antibody fragment comprises a single chain variable fragment, a single domain antibody, or a Fab fragment.

Embodiment 9 comprises an isolated polypeptide or polypeptide complex of embodiment 8, wherein A₁ is the single chain variable fragment (scFv).

Embodiment 10 comprises an isolated polypeptide or polypeptide complex of embodiment 9, wherein the scFv comprises a scFv heavy chain polypeptide and a scFv light chain polypeptide.

Embodiment 11 comprises an isolated polypeptide or polypeptide complex of embodiment 8, wherein A₁ is the single domain antibody.

Embodiment 12 comprises an isolated polypeptide or polypeptide complex of embodiment 2, wherein the antibody or antibody fragment comprises a single chain variable fragment (scFv), a heavy chain variable domain (VH domain), a light chain variable domain (VL domain), or a variable domain (VHH) of a camelid derived single domain antibody.

Embodiment 13 comprises an isolated polypeptide or polypeptide complex of any one of embodiments 1-10 and 12, wherein A₁ comprises an anti-CD3e single chain variable fragment.

Embodiment 14 comprises an isolated polypeptide or polypeptide complex of any one of embodiments 1-10 and 12-13, wherein A₁ comprises an anti-CD3e single chain variable fragment that has a K_{D} binding of 1 μM or less to CD3 on CD3 expressing cells.

Embodiment 15 comprises an isolated polypeptide or polypeptide complex of any one of embodiments 1-14, wherein the effector cell antigen comprises CD3.

Embodiment 16 comprises an isolated polypeptide or polypeptide complex of any one of embodiments 1-15, wherein A₁ comprises a variable light chain and variable heavy chain each of which is capable of specifically binding to human CD3.

Embodiment 17 comprises an isolated polypeptide or polypeptide complex of any one of embodiments 1-16, wherein A₁ comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, 15865, 15865v12, 15865v16, and 15865v19.

Embodiment 18 comprises an isolated polypeptide or polypeptide complex of any one of embodiments 1-17, wherein the isolated polypeptide or polypeptide complex of Formula I binds to an effector cell when L₁ is cleaved by the tumor specific protease.

Embodiment 19 comprises an isolated polypeptide or polypeptide complex of any one of embodiments 1-17, wherein the isolated polypeptide or polypeptide complex of Formula I binds to an effector cell when L₁ is cleaved by the tumor specific protease and A₁ binds to the effector cell.

Embodiment 20 comprises an isolated polypeptide or polypeptide complex of any one of embodiments 18-19, wherein the effector cell is a T cell.

Embodiment 21 comprises an isolated polypeptide or polypeptide complex of any one of embodiments 18-20, wherein A₁ binds to a polypeptide that is part of a TCR-CD3 complex on the effector cell.

Embodiment 22 comprises an isolated polypeptide or polypeptide complex of embodiment 21, wherein the polypeptide that is part of the TCR-CD3 complex is human CD3ε.

Embodiment 23 comprises an isolated polypeptide or polypeptide complex of any one of embodiments 9-10 and 12-22, wherein the effector cell antigen comprises CD3, wherein the scFv comprises complementarity determining regions (CDRs): HC-CDR1, HC-CDR2, and HC-CDR3, wherein the HC-CDR1, the HC-CDR2, and the HC-CDR3 of the scFv comprise: HC-CDR1: SEQ ID NO: 1, HC-CDR2: SEQ ID NO: 2, and HC-CDR3: SEQ ID NO: 3; and the scFv comprises CDRs: LC-CDR1, LC-CDR2, and LC-CDR3, wherein the LC-CDR1, the LC-CDR2, and the LC-CDR3 of the scFv comprise LC-CDR1: SEQ ID NO: 4, LC-CDR2: SEQ ID NO:5, and LC-CDR3: SEQ ID NO: 6.

Embodiment 24 comprises an isolated polypeptide or polypeptide complex of any one of embodiments 1-23, wherein the effector cell antigen comprises CD3, wherein A₁ comprises complementarity determining regions (CDRs): HC-CDR1, HC-CDR2, and HC-CDR3, wherein the HC-CDR1, the HC-CDR2, and the HC-CDR3 of A₁ comprise: HC-CDR1: SEQ ID NO: 1, HC-CDR2: SEQ ID NO: 2, and HC-CDR3: SEQ ID NO: 3; and A₁ comprises CDRs: LC-CDR1, LC-CDR2, and LC-CDR3, wherein the LC-CDR1, the LC-CDR2, and the LC-CDR3 of A₁ comprise LC-CDR1: SEQ ID NO: 4, LC-CDR2: SEQ ID NO:5, and LC-CDR3: SEQ ID NO: 6.

Embodiment 25 comprises an isolated polypeptide or polypeptide complex of any one of embodiments 9-10 and 12-22, wherein the effector cell antigen comprises CD3, and the scFv comprises an amino acid sequence according to SEQ ID NO: 13.

Embodiment 26 comprises an isolated polypeptide or polypeptide complex of any one of embodiments 9-10 and 12-22, wherein the effector cell antigen comprises CD3, wherein the scFv comprises complementarity determining regions (CDRs): HC-CDR1, HC-CDR2, and HC-CDR3, wherein the HC-CDR1, the HC-CDR2, and the HC-CDR3 of the scFv comprise: HC-CDR1: SEQ ID NO: 7, HC-CDR2: SEQ ID NO: 8, and HC-CDR3: SEQ ID NO: 9; and the scFv comprises CDRs: LC-CDR1, LC-CDR2, and LC-CDR3, wherein the LC-CDR1, the LC-CDR2, and the LC-CDR3 of the scFv comprise LC-CDR1: SEQ ID NO: 10, LC-CDR2: SEQ ID NO: 11, and LC-CDR3: SEQ ID NO: 12.

Embodiment 27 comprises an isolated polypeptide or polypeptide complex of any one of embodiments 1-22, wherein the effector cell antigen comprises CD3, wherein A₁ comprises complementarity determining regions (CDRs): HC-CDR1, HC-CDR2, and HC-CDR3, wherein the HC-CDR1, the HC-CDR2, and the HC-CDR3 of A₁ comprise: HC-CDR1: SEQ ID NO: 7, HC-CDR2: SEQ ID NO: 8, and HC-CDR3: SEQ ID NO: 9; and A₁ comprises CDRs: LC-CDR1, LC-CDR2, and LC-CDR3, wherein the LC-CDR1, the LC-CDR2, and the LC-CDR3 of A₁ comprise LC-CDR1: SEQ ID NO: 10, LC-CDR2: SEQ ID NO: 11, and LC-CDR3: SEQ ID NO: 12.

Embodiment 28 comprises an isolated polypeptide or polypeptide complex of any one of embodiments 9-10 and 12-22, wherein the effector cell antigen comprises CD3, and the scFv comprises an amino acid sequence according to SEQ ID NO: 14.

Embodiment 29 comprises an isolated polypeptide or polypeptide complex of any one of embodiments 1-28, wherein second antigen recognizing molecule comprises an antibody or antibody fragment.

Embodiment 30 comprises an isolated polypeptide or polypeptide complex of embodiment 29, wherein the antibody or antibody fragment thereof comprises a single chain variable fragment, a single domain antibody, or a Fab.

Embodiment 31 comprises an isolated polypeptide or polypeptide complex of embodiment 30, wherein the antibody or antibody fragment thereof comprises a single chain variable fragment (scFv), a heavy chain variable domain (VH domain), a light chain variable domain (VL domain), a variable domain (VHH) of a camelid derived single domain antibody.

Embodiment 32 comprises an isolated polypeptide or polypeptide complex of any one of embodiments 29-31, wherein the antibody or antibody fragment thereof is humanized or human.

Embodiment 33 comprises an isolated polypeptide or polypeptide complex of embodiment 30, wherein A₂ is the Fab.

Embodiment 34 comprises an isolated polypeptide or polypeptide complex of embodiment 33, wherein the Fab comprises (a) a Fab light chain polypeptide and (b) a Fab heavy chain polypeptide.

Embodiment 35 comprises an isolated polypeptide or polypeptide complex of any one of embodiments 33-34, wherein the Fab comprises complementarity determining regions (CDRs): HC-CDR1, HC-CDR2, and HC-CDR3, wherein the HC-CDR1, the HC-CDR2, and the HC-CDR3 of the Fab comprise: HC-CDR1: SEQ ID NO: 15, HC-CDR2: SEQ ID NO: 16, and HC-CDR3: SEQ ID NO: 17; and the Fab comprises CDRs: LC-CDR1, LC-CDR2, and LC-CDR3, wherein the LC-CDR1, the LC-CDR2, and the LC-CDR3 of the Fab comprise LC-CDR1: SEQ ID NO: 18, LC-CDR2: SEQ ID NO: 19, and LC-CDR3: SEQ ID NO: 20.

Embodiment 36 comprises an isolated polypeptide or polypeptide complex of any one of embodiments 1-35, wherein A₂ comprises complementarity determining regions (CDRs): HC-CDR1, HC-CDR2, and HC-CDR3, wherein the HC-CDR1, the HC-CDR2, and the HC-CDR3 of A₂ comprise: HC-CDR1: SEQ ID NO: 15, HC-CDR2: SEQ ID NO: 16, and HC-CDR3: SEQ ID NO: 17; and A₂ comprises CDRs: LC-CDR1, LC-CDR2, and LC-CDR3, wherein the LC-CDR1, the LC-CDR2, and the LC-CDR3 of A₂ comprise LC-CDR1: SEQ ID NO: 18, LC-CDR2: SEQ ID NO: 19, and LC-CDR3: SEQ ID NO: 20.

Embodiment 37 comprises an isolated polypeptide or polypeptide complex of embodiment 34, wherein the Fab light chain polypeptide comprises an amino acid sequence according to SEQ ID NO: 21.

Embodiment 38 comprises an isolated polypeptide or polypeptide complex of embodiment 34 and 37, wherein Fab heavy chain polypeptide comprises an amino acid sequence according to SEQ ID NO: 22.

Embodiment 39 comprises an isolated polypeptide or polypeptide complex of any one of embodiments 34 and 37-38, wherein the Fab light chain polypeptide of A₂ is bound to a C-terminus of the single chain variable fragment (scFv) of A₁.

Embodiment 40 comprises an isolated polypeptide or polypeptide complex of any one of embodiments 34 and 37-38, wherein the Fab heavy chain polypeptide of A₂ is bound to a C-terminus of the single chain variable fragment (scFv) A₁.

Embodiment 41 comprises an isolated polypeptide or polypeptide complex of any one of embodiments 34, 37-38 and 40, wherein the Fab light chain polypeptide of A₂ is bound to a N-terminus of the single chain variable fragment (scFv) of A₁.

Embodiment 42 comprises an isolated polypeptide or polypeptide complex of any one of embodiments 34 and 37-39, wherein the Fab heavy chain polypeptide of A₂ is bound to a N-terminus of the single chain variable fragment (scFv) A₁.

Embodiment 43 comprises an isolated polypeptide or polypeptide complex of any one of embodiments 34-38, wherein the Fab heavy chain polypeptide of A₂ is bound to the scFv heavy chain polypeptide of A₁.

Embodiment 44 comprises an isolated polypeptide or polypeptide complex of any one of embodiments 34-38, wherein the Fab light chain polypeptide of A₂ is bound to the scFv heavy chain polypeptide of A₁.

Embodiment 45 comprises an isolated polypeptide or polypeptide complex of any one of embodiments 34-48, wherein the Fab heavy chain polypeptide of A₂ is bound to the scFv light chain polypeptide of A₁.

Embodiment 46 comprises an isolated polypeptide or polypeptide complex of any one of embodiments 34-38, wherein the Fab light chain polypeptide of A₂ is bound to the scFv light chain polypeptide of A₁.

Embodiment 47 comprises an isolated polypeptide or polypeptide complex of any one of embodiments 1-46, wherein A₂ further comprises P₂ and L₂, wherein P₂ comprises a peptide that binds to A₂; and L₂ comprises a linking moiety that connects A₂ to P₂ and is a substrate for a tumor specific protease.

Embodiment 48 comprises an isolated polypeptide or polypeptide complex of embodiment 47, wherein the isolated polypeptide or polypeptide complex is according to Formula Ia: P₂-L₂-A₂-A₁-L₁-P₁-H₁.

Embodiment 49 comprises an isolated polypeptide or polypeptide complex of any one of embodiments 47-48, wherein the Fab heavy chain polypeptide of A₂ is bound to the scFv heavy chain polypeptide of A₁ and L₂ is bound to the Fab light chain polypeptide of A₂.

Embodiment 50 comprises an isolated polypeptide or polypeptide complex of any one of embodiments 47-48, wherein the Fab light chain polypeptide of A₂ is bound to the scFv heavy chain polypeptide of A₁ and L₂ is bound to the Fab heavy chain polypeptide of A₂.

Embodiment 51 comprises an isolated polypeptide or polypeptide complex of any one of embodiments 47-48, wherein the Fab heavy chain polypeptide of A₂ is bound to the scFv light chain polypeptide of A₁ and L₂ is bound to the Fab light chain polypeptide of A₂.

Embodiment 52 comprises an isolated polypeptide or polypeptide complex of any one of embodiments 47-48, wherein the Fab light chain polypeptide of A₂ is bound to the scFv light chain polypeptide of A₁ and L₂ is bound to the Fab heavy chain polypeptide of A₂.

Embodiment 53 comprises an isolated polypeptide or polypeptide complex of any one of embodiments 1-52, wherein P₁ impairs binding of A₁ to the effector cell antigen.

Embodiment 54 comprises an isolated polypeptide or polypeptide complex of any one of embodiments 1-53, wherein P₁ is bound to A₁ through ionic interactions, electrostatic interactions, hydrophobic interactions, Pi-stacking interactions, and H-bonding interactions, or a combination thereof.

Embodiment 55 comprises an isolated polypeptide or polypeptide complex of any one of embodiments 1-54, wherein P₁ has less than 70% sequence homology to the effector cell antigen.

Embodiment 56 comprises an isolated polypeptide or polypeptide complex of any one of embodiments 47-55, wherein P₂ impairs binding of A₂ to TROP2.

Embodiment 57 comprises an isolated polypeptide or polypeptide complex of any one of embodiments 47-56, wherein P₂ is bound to A₂ through ionic interactions, electrostatic interactions, hydrophobic interactions, Pi-stacking interactions, and H-bonding interactions, or a combination thereof.

Embodiment 58 comprises an isolated polypeptide or polypeptide complex of any one of embodiments 47-57, wherein P₂ is bound to A₂ at or near an antigen binding site.

Embodiment 59 comprises an isolated polypeptide or polypeptide complex of any one of embodiments 47-58, wherein P₂ has less than 70% sequence homology to TROP2.

Embodiment 60 comprises an isolated polypeptide or polypeptide complex of any one of embodiments 47-59, wherein P₁ or P₂ comprises a peptide sequence of at least 10 amino acids in length.

Embodiment 61 comprises an isolated polypeptide or polypeptide complex of any one of embodiments 47-60, wherein P₁ or P₂ comprises a peptide sequence of at least 10 amino acids in length and no more than 20 amino acids in length.

Embodiment 62 comprises an isolated polypeptide or polypeptide complex of any one of embodiments 47-59, wherein P₁ or P₂ comprises a peptide sequence of at least 16 amino acids in length.

Embodiment 63 comprises an isolated polypeptide or polypeptide complex of any one of embodiments 47-60 and 62, wherein P₁ or P₂ comprises a peptide sequence of no more than 40 amino acids in length.

Embodiment 64 comprises an isolated polypeptide or polypeptide complex of any one of embodiments 47-63, wherein P₁ or P₂ comprises at least two cysteine amino acid residues.

Embodiment 65 comprises an isolated polypeptide or polypeptide complex of any one of embodiments 47-64, wherein P₁ or P₂ comprises a cyclic peptide or a linear peptide.

Embodiment 66 comprises an isolated polypeptide or polypeptide complex of any one of embodiments 47-65, wherein P₁ or P₂ comprises a cyclic peptide.

Embodiment 67 comprises an isolated polypeptide or polypeptide complex of any one of embodiments 47-65, wherein P₁ or P₂ comprises a linear peptide.

Embodiment 68 comprises an isolated polypeptide or polypeptide complex of any one of embodiments 1-67, wherein P₁ comprises at least two cysteine amino acid residues.

Embodiment 69 comprises an isolated polypeptide or polypeptide complex of any one of embodiments 1-68, wherein P₁ comprises an amino acid sequence according to SEQ ID NO: 26, 27, or 122.

Embodiment 70 comprises an isolated polypeptide or polypeptide complex of any one of embodiments 47-68, wherein P₂ comprises an amino acid sequence according to any one of SEQ ID NOs: 23-25.

Embodiment 71 comprises an isolated polypeptide or polypeptide complex of any one of embodiments 47-68, wherein P₂ comprises an amino acid sequence according to X1-X₂-X₃-X₄-C-X₆-X₇-X₈-X₉-X₁₀-C-X₁₂-X₁₃-X₁₄ and X₁ is selected from N, D, S, Y, A, F, H, T, L, and V; X₂ is selected from S, T, D, A, H, V, Y, N, F, I, and L; X₃ is selected from L, I, and V; X₄ is selected from F, L, V, M, W, I, Y, and H; X₆ is selected from V, F, L, I, and W; X₇ is selected from K, R, Q, N, H, and M; X₈ is selected from N and K; X₉ is selected from L, V, and I; X₁₀ is selected from Y, W, F, Q, and L; X₁₂ is selected from W and V; X₁₃ is selected from I, N, H, T, V, Y, and D; X₁₄ is selected from D, V, A, S, I, T, N, Y, H, and P.

Embodiment 72 comprises an isolated polypeptide or polypeptide complex of embodiment 71, wherein X₁ is selected from N, D, S, Y, A, F, and T; X₂ is selected from S, T, D, A, H, V, Y, and N; X₃ is L; X₄ is selected from F, L, V, M, and W; X₆ is selected from V, F, and L; X₇ is selected from K, R, Q, and N; X₈ is selected from N and K; X₉ is selected from L, V, and I; X₁₀ is selected from Y, W, and F; X₁₂ is W; X₁₃ is selected from I, N, H, and T; X₁₄ is selected from D, V, A, S, I, T, and N.

Embodiment 73 comprises an isolated polypeptide or polypeptide complex of any one of embodiments 71-72, wherein X₁ is selected from N, D, S, and Y; X₂ is selected from S, T, and D; X₃ is L; X₄ is selected from F, L, and V; X₆ is selected from V and F; X₇ is selected from K, R, and Q; X₈ is N; X₉ is selected from L and V; X₁₀ is selected from Y and W; X₁₂ is W; X₁₃ is selected from I, N, and H; X₁₄ is selected from D, V, A, and S.

Embodiment 74 comprises an isolated polypeptide or polypeptide complex of any one of embodiments 47-68, wherein P₂ comprises an amino acid sequence according to an amino acid sequence according to J₁-J₂-J₃-C-J₅-J₆-J₇-J₈-W-J₁₀-J₁₁-C-J₁₃-J₁₄ and J₁ is selected from V, I, L, P, E, F, and M; J₂ is selected from D and N; J₃ is selected from F and W; J₈ is selected from A, E, S, R, K, Y, L, Q, G, M, F, T, W, and D; J₆ is selected from L, M, I, V, F, T, R, and S; J₇ is selected from Y, F, and N; J₈ is selected from N, R, D, H, K, Q, S, G, A, E, and M; J₁₀ is selected from P and R; J₁₁ is selected from V and I; J₁₃ is selected from D, G, N, R, S, Q, Y, T, A, E, L, V, K, M, I, H, F, and W; J₁₄ is selected from T, S, Q, L, D, N, A, E, K, M, V, R, I, H, P, V, and W; or P₂ comprises an amino acid sequence according to B₁-B₂-B₃-C-B₈-B₆-B₇-B₈-W-B₁₀-B₁₁-C-B₁₃-B₁₄ and B₁ is selected from V, I, L, P, E, F, and M; B₂ is selected from D and N; B₃ is selected from F and W; B₈ is selected from A, E, S, R, K, Y, L, Q, G, M, F, T, W, and D; B₆ is selected from L, M, I, V, F, T, R, and S; B₇ is selected from Y, F, and N; B₈ is selected from N, R, D, H, K, Q, S, G, A, E, and M; B₁₀ is selected from P and R; B₁₁ is selected from V and I; B₁₃ is selected from D, G, N, R, S, Q, Y, T, A, E, L, V, K, M, I, H, F, and W; B₁₄ is selected from T, S, Q, L, D, N, A, E, K, M, V, R, I, H, P, V, G, and W.

Embodiment 75 comprises an isolated polypeptide or polypeptide complex of embodiment 74, wherein J₁ is selected from V, I, and L; J₂ is D; J₃ is F; J₈ is selected from A, E, S, R, K, Y, and L; J₆ is selected from L, M, and I; J₇ is Y; J₈ is selected from N, R, D, H, K, Q, S, and G; J₁₀ is P; J₁, is selected from V and I; J₁₃ is selected from D, G, N, R, S, Q, Y, T, and A; J₁₄ is selected from T, S, Q, L, D, N, A, and E; and B₁ is selected from V, I, and L; B₂ is D; B₃ is F; B₈ is selected from A, E, S, R, K, Y, M, G, and L; B₆ is selected from L, M, and I; B₇ is Y; B₈ is selected from N, R, D, H, K, Q, S, and G; B₁₀ is P; B₁₁ is selected from V and I; B₁₃ is selected from D, G, N, R, S, Q, Y, T, H, and A; B₁₄ is selected from T, S, Q, L, D, N, A, G, and E.

Embodiment 76 comprises an isolated polypeptide or polypeptide complex of any one of embodiments 74-75, wherein J₁ is selected from V, I, and L; J₂ is D; J₃ is F; J₈ is selected from A, E, S, and R; J₆ is selected from L, M, and I; J₇ is Y; J₈ is selected from N, R, and D; J₁₀ is P; J₁₁ is selected from V and I; J₁₃ is selected from D, G, N, R, S, and Q; J₁₄ is selected from T, S, Q, and L; and B₁ is selected from V, I, and L; B₂ is D; B₃ is F; B₈ is selected from A, E, S, K, M, G, and R; B₆ is selected from L, M, and I; B₇ is Y; B₈ is selected from N, R, S, H, and D; B₁₀ is P; B₁₁ is selected from V and I; B₁₃ is selected from D, G, N, R, S, H, A, Y, and Q; B₁₄ is selected from T, S, Q, G, and L.

Embodiment 77 comprises an isolated polypeptide or polypeptide complex of any one of embodiments 47-68, wherein P₂ comprises the amino acid sequences according to SEQ ID NOs: 133-145.

Embodiment 78 comprises an isolated polypeptide or polypeptide complex of any one of embodiments 47-68, wherein P₂ comprises the amino acid sequences according to SEQ ID NOs: 146-158.

Embodiment 79 comprises an isolated polypeptide or polypeptide complex of any one of embodiments 47-68, wherein P₂ comprises an amino acid sequence according to any of the sequences of Table 27.

Embodiment 80 comprises an isolated polypeptide or polypeptide complex of any one of embodiments 71-73, or 79, wherein P₂ comprises the amino acid sequences according to SEQ ID NOs: 159-178.

Embodiment 81 comprises an isolated polypeptide or polypeptide complex of any one of embodiments 47-68, wherein P₂ comprises an amino acid sequence according to any of the sequences of Table 29.

Embodiment 82 comprises an isolated polypeptide or polypeptide complex of any one of embodiments 74-76, or 81, wherein P₂ comprises the amino acid sequences according to SEQ ID NOs: 179-201.

Embodiment 83 comprises an isolated polypeptide or polypeptide complex of any one of embodiments 74-76, or 81, wherein P₂ comprises the amino acid sequence according to SEQ ID NO: 24 or a peptide sequence that has 1, 2, or 3, amino acid substitutions, additions, or deletions relative to the amino acid sequence of SEQ ID NO: 24.

Embodiment 84 comprises an isolated polypeptide or polypeptide complex of any one of embodiments 74-76, or 81, wherein P₂ comprises the amino acid sequence according to SEQ ID NO: 181 or a peptide sequence that has 1, 2, or 3, amino acid substitutions, additions, or deletions relative to the amino acid sequence of SEQ ID NO: 181.

Embodiment 85 comprises an isolated polypeptide or polypeptide complex of any one of embodiments 74-76, or 81, wherein P₂ comprises the amino acid sequence according to SEQ ID NO: 186 or a peptide sequence that has 1, 2, or 3, amino acid substitutions, additions, or deletions relative to the amino acid sequence of SEQ ID NO: 186.

Embodiment 86 comprises an isolated polypeptide or polypeptide complex of any one of embodiments 74-76, or 81, wherein P₂ comprises the amino acid sequence according to SEQ ID NO: 24.

Embodiment 87 comprises an isolated polypeptide or polypeptide complex of any one of embodiments 74-76, or 81, wherein P₂ comprises the amino acid sequence according to SEQ ID NO: 181.

Embodiment 88 comprises an isolated polypeptide or polypeptide complex of any one of embodiments 74-76, or 81, wherein P₂ comprises the amino acid sequence according to SEQ ID NO: 186.

Embodiment 89 comprises an isolated polypeptide or polypeptide complex of any one of embodiments 1-68 and 70-88, wherein P₁ comprises an amino acid sequence according to Z₁-Z₂-C-Z₄-P-Z₆—Z₇-Z₈-Z₉-Z₁₀-Z₁₁-Z₁₂-C-Z₁₄ and Z₁ is selected from D, Y, F, I, N, V, H, L, A, T, S, and P; Z₂ is selected from D, Y, L, F, I, N, A, V, H, T, and S; Z₄ is selected from G and W; Z₆ is selected from E, D, V, and P; Z₇ is selected from W, L, F, V, G, M, I, and Y; Z₈ is selected from E, D, P, and Q; Z₉ is selected from E, D, Y, V, F, W, P, L, and Q; Z₁₀ is selected from S, D, Y, T, I, F, V, N, A, P, L, and H; Z₁₁ is selected from I, Y, F, V, L, T, N, S, D, A, and H; Z₁₂ is selected from F, D, Y, L, I, V, A, N, T, P, S, and H; Z₁₄ is selected from D, Y, N, F, I, P, V, A, T, H, L and S; or P₁ comprises an amino acid sequence according to U₁-U₂-C-U₄-P-U₆—U₇-U₈-U₉-U₁₀-U₁₁-U₁₂-C-U₁₄ and U₁ is selected from D, Y, F, I, N, V, H, L, A, T, S, and P; U₂ is selected from D, Y, L, F, I, N, A, V, H, T, and S; U₄ is selected from G and W; U₆ is selected from E, D, V, and P; U₇ is selected from W, L, F, V, G, M, I, and Y; U₈ is selected from E, D, P, and Q; U₉ is selected from E, D, Y, V, F, W, P, L, and Q; U₁₀ is selected from S, D, Y, T, I, F, V, N, A, P, L, and H; U₁₁ is selected from I, Y, F, V, L, T, N, S, D, A, and H; U₁₂ is selected from F, D, Y, L, I, V, A, N, T, P, S, G, and H; U₁₄ is selected from D, Y, N, F, I, P, V, A, T, H, L, M, and S.

Embodiment 90 comprises an isolated polypeptide or polypeptide complex of embodiment 89, wherein Z₁ is selected from D, Y, F, I, and N; Z₂ is selected from D, Y, L, F, I, and N; Z₄ is selected from G and W; Z₆ is selected from E and D; Z₇ is selected from W, L, F, and V; Z₈ is selected from E and D; Z₉ is selected from E, D, Y, and V; Z₁₀ is selected from S, D, Y, T, and I; Z₁ is selected from I, Y, F, V, L, and T; Z₁₂ is selected from F, D, Y, L, I, V, A, and N; Z₁₄ is selected from D, Y, N, F, I, and P; and U₁ is selected from D, Y, F, I, V, and N; U₂ is selected from D, Y, L, F, I, and N; U₄ is selected from G and W; U₆ is selected from E and D; U₇ is selected from W, L, F, G, and V; U₈ is selected from E and D; U₉ is selected from E, D, Y, and V; U₁₀ is selected from S, D, Y, T, and I; U₁₁ is selected from I, Y, F, V, L, and T; U₁₂ is selected from F, D, Y, L, I, V, A, G, and N; U₁₄ is selected from D, Y, N, F, I, M, and P.

Embodiment 91 comprises an isolated polypeptide or polypeptide complex of any one of embodiments 89-90, wherein Z₁ is selected D, Y, and F; Z₂ is selected from D, Y, L, and F; Z₄ is selected from G and W; Z₆ is selected from E and D; Z₇ is selected from W, L, and F; Z₈ is selected from E and D; Z₉ is selected from E and D; Z₁₀ is selected from S, D, and Y; Z₁ is selected from I, Y, and F; Z₁₂ is selected from F, D, Y, and L; Z₁₄ is selected from D, Y, and N; and U₁ is selected from D, Y, V, and F; U₂ is selected from D, Y, L, and F; U₄ is selected from G and W; U₆ is selected from E and D; U₇ is selected from W, L, G, and F; U₈ is selected from E and D; U₉ is selected from E and D; U₁₀ is selected from S, D, T, and Y; U₁₁ is selected from I, Y, V, L, and F; U₁₂ is selected from F, D, Y, G, A, and L; U₁₄ is selected from D, Y, M, and N.

Embodiment 92 comprises an isolated polypeptide or polypeptide complex of any one of embodiments 1-68 and 70-88, wherein P₁ comprises the amino acid sequences according to SEQ ID NOs: 202-228.

Embodiment 93 comprises an isolated polypeptide or polypeptide complex of any one of embodiments 1-68 and 70-88, wherein P₁ comprises an amino acid sequences according to any of the sequences of Table 35.

Embodiment 94 comprises an isolated polypeptide or polypeptide complex of any one of embodiments 89-91 or 93, wherein P₁ comprises the amino acid sequences according to SEQ ID NOs: 229-240.

Embodiment 95 comprises an isolated polypeptide or polypeptide complex of any one of embodiments 89-91 or 93, wherein P₁ comprises the amino acid sequence according to SEQ ID NO: 239 or a peptide sequence that has 1, 2, or 3, amino acid substitutions, additions, or deletions relative to the amino acid sequence of SEQ ID NO: 239.

Embodiment 96 comprises an isolated polypeptide or polypeptide complex of any one of embodiments 89-91 or 93, wherein P₁ comprises the amino acid sequence according to SEQ ID NO: 27 or a peptide sequence that has 1, 2, or 3, amino acid substitutions, additions, or deletions relative to the amino acid sequence of SEQ ID NO: 27.

Embodiment 97 comprises an isolated polypeptide or polypeptide complex of any one of embodiments 1-68 and 70-88, wherein P₁ comprises the amino acid sequence according to SEQ ID NO: 26 or a peptide sequence that has 1, 2, or 3, amino acid substitutions, additions, or deletions relative to the amino acid sequence of SEQ ID NO: 26.

Embodiment 98 comprises an isolated polypeptide or polypeptide complex of any one of embodiments 89-91 or 93, wherein P₁ comprises the amino acid sequence according to SEQ ID NO: 239.

Embodiment 99 comprises an isolated polypeptide or polypeptide complex of any one of embodiments 89-91 or 93, wherein P₁ comprises the amino acid sequence according to SEQ ID NO: 27.

Embodiment 100 comprises an isolated polypeptide or polypeptide complex of any one of embodiments 1-68 and 70-88, wherein P₁ comprises the amino acid sequence according to SEQ ID NO: 26.

Embodiment 101 comprises an isolated polypeptide or polypeptide complex of any one of embodiments 1-5, 8-40, and 43-100, wherein L₁ is bound to N-terminus of A₁.

Embodiment 102 comprises an isolated polypeptide or polypeptide complex of any one of embodiments 1-3, 6-38, and 41-100, wherein L₁ is bound to C-terminus of A₁.

Embodiment 103 comprises an isolated polypeptide or polypeptide complex of any one of embodiments 1-38, and 41-100, wherein L₂ is bound to N-terminus of A₂.

Embodiment 104 comprises an isolated polypeptide or polypeptide complex of any one of embodiments 1-40, and 43-100, wherein L₂ is bound to C-terminus of A₂.

Embodiment 105 comprises an isolated polypeptide or polypeptide complex of any one of embodiments 1-104, wherein L₁ or L₂ is a peptide sequence having at least 5 to no more than 50 amino acids.

Embodiment 106 comprises an isolated polypeptide or polypeptide complex of any one of embodiments 1-105, wherein L₁ or L₂ is a peptide sequence having at least 10 to no more than 30 amino acids.

Embodiment 107 comprises an isolated polypeptide or polypeptide complex of any one of embodiments 1-106, wherein L₁ or L₂ is a peptide sequence having at least 10 amino acids.

Embodiment 108 comprises an isolated polypeptide or polypeptide complex of any one of embodiments 1-107, wherein L₁ or L₂ is a peptide sequence having at least 18 amino acids.

Embodiment 109 comprises an isolated polypeptide or polypeptide complex of any one of embodiments 1-108, wherein L₁ or L₂ is a peptide sequence having at least 26 amino acids.

Embodiment 110 comprises an isolated polypeptide or polypeptide complex of any one of embodiments 1-109, wherein L₁ or L₂ has a formula comprising (G₂S)ₙ (SEQ ID NO: 243), wherein n is an integer from 1 to 3.

Embodiment 111 comprises an isolated polypeptide or polypeptide complex of any one of embodiments 1-109, wherein L₁ has a formula selected from the group consisting of (G₂S)ₙ, (GS)ₙ, (GSGGS)ₙ (SEQ ID NO: 62), (GGGS)ₙ (SEQ ID NO: 63), (GGGGS)ₙ (SEQ ID NO: 64), and (GSSGGS)ₙ (SEQ ID NO: 65), wherein n is an integer of at least 1.

Embodiment 112 comprises an isolated polypeptide or polypeptide complex of any one of embodiments 1-111, wherein P₁ becomes unbound from A₁ when L₁ is cleaved by the tumor specific protease thereby exposing A₁ to the effector cell antigen.

Embodiment 113 comprises an isolated polypeptide or polypeptide complex of any one of embodiments 1-112, wherein P₂ becomes unbound from A₂ when L₂ is cleaved by the tumor specific protease thereby exposing A₂ to TROP2.

Embodiment 114 comprises an isolated polypeptide or polypeptide complex of any one of embodiments 1-113, wherein the tumor specific protease is selected from the group consisting of a matrix metalloprotease (MMP), serine protease, cysteine protease, threonine protease, and aspartic protease.

Embodiment 115 comprises an isolated polypeptide or polypeptide complex of embodiment 114, wherein the matrix metalloprotease comprises MMP2, MMP7, MMP9, MMP13, or MMP14.

Embodiment 116 comprises an isolated polypeptide or polypeptide complex of embodiment 114, wherein the serine protease comprises matriptase (MTSP1), urokinase, or hepsin.

Embodiment 117 comprises an isolated polypeptide or polypeptide complex of any one of embodiments 1-116, wherein L₁ or L₂ comprises a urokinase cleavable amino acid sequence, a matriptase cleavable amino acid sequence, matrix metalloprotease cleavable amino acid sequence, or a legumain cleavable amino acid sequence.

Embodiment 118 comprises an isolated polypeptide or polypeptide complex of any one of embodiments 1-117, wherein L₁ or L₂ comprises an amino acid sequence according to SEQ ID NO: 31 or 32.

Embodiment 119 comprises an isolated polypeptide or polypeptide complex of any one of embodiments 1-117, wherein L₁ or L₂ comprises an amino acid sequence according to SEQ ID NO: 58 or 59.

Embodiment 120 comprises an isolated polypeptide or polypeptide complex of any one of embodiments 1-117, wherein L₁ or L₂ comprises an amino acid sequence according to any one of SEQ ID NOs: 28-61.

Embodiment 121 comprises an isolated polypeptide or polypeptide complex of any one of embodiments 1-117, wherein L₁ or L₂ comprises an amino acid sequence of Linker 25 (ISSGLLSGRSDAG) (SEQ ID NO: 54), Linker 26 (AAGLLAPPGGLSGRSDAG) (SEQ ID NO: 55), Linker 27 (SPLGLSGRSDAG) (SEQ ID NO: 56), or Linker 28 (LSGRSDAGSPLGLAG) (SEQ ID NO: 57), or an amino acid sequence that has 1, 2, or 3 amino acid substitutions, additions, or deletions relative to the amino acid sequence of Linker 25, Linker 26, Linker 27, or Linker 28.

Embodiment 122 comprises an isolated polypeptide or polypeptide complex of any one of embodiments 1-121, wherein H₁ comprises a polymer.

Embodiment 123 comprises an isolated polypeptide or polypeptide complex of embodiment 122, wherein the polymer is polyethylene glycol (PEG).

Embodiment 124 comprises an isolated polypeptide or polypeptide complex of any one of embodiments 1-121, wherein H₁ comprises albumin.

Embodiment 125 comprises an isolated polypeptide or polypeptide complex of any one of embodiments 1-121, wherein H₁ comprises an Fc domain.

Embodiment 126 comprises an isolated polypeptide or polypeptide complex of embodiment 124, wherein the albumin is serum albumin.

Embodiment 127 comprises an isolated polypeptide or polypeptide complex of embodiment 124, wherein the albumin is human serum albumin.

Embodiment 128 comprises an isolated polypeptide or polypeptide complex of any one of embodiments 1-121, wherein H₁ comprises a polypeptide, a ligand, or a small molecule.

Embodiment 129 comprises an isolated polypeptide or polypeptide complex of embodiment 128, wherein the polypeptide, the ligand or the small molecule binds serum protein or a fragment thereof, a circulating immunoglobulin or a fragment thereof, or CD35/CR1.

Embodiment 130 comprises an isolated polypeptide or polypeptide complex of embodiment 129, wherein the serum protein comprises a thyroxine-binding protein, a transthyretin, a 1-acid glycoprotein, a transferrin, transferrin receptor or a transferrin-binding portion thereof, a fibrinogen, or an albumin.

Embodiment 131 comprises an isolated polypeptide or polypeptide complex of any one of embodiments 129, wherein the circulating immunoglobulin molecule comprises IgG1, IgG2, IgG3, IgG4, slgA, IgM or IgD.

Embodiment 132 comprises an isolated polypeptide or polypeptide complex of embodiment 129, wherein the serum protein is albumin.

Embodiment 133 comprises an isolated polypeptide or polypeptide complex of any one of embodiments 128-132, wherein the polypeptide is an antibody.

Embodiment 134 comprises an isolated polypeptide or polypeptide complex of embodiment 133, wherein the antibody comprises a single domain antibody, a single chain variable fragment, or a Fab.

Embodiment 135 comprises an isolated polypeptide or polypeptide complex of embodiment 134, wherein the single domain antibody comprises a single domain antibody that binds to albumin.

Embodiment 136 comprises an isolated polypeptide or polypeptide complex of any one of embodiments 134-135, wherein the single domain antibody is a human or humanized antibody.

Embodiment 137 comprises an isolated polypeptide or polypeptide complex of embodiment 134, wherein the single domain antibody is 645gH1gL1.

Embodiment 138 comprises an isolated polypeptide or polypeptide complex of embodiment 134, wherein the single domain antibody is 645dsgH5gL4.

Embodiment 139 comprises an isolated polypeptide or polypeptide complex of embodiment 134, wherein the single domain antibody is 23-13-A01-sc02.

Embodiment 140 comprises an isolated polypeptide or polypeptide complex of embodiment 134, wherein the single domain antibody is A10m3 or a fragment thereof.

Embodiment 141 comprises an isolated polypeptide or polypeptide complex of embodiment 134, wherein the single domain antibody is DOM7r-31.

Embodiment 142 comprises an isolated polypeptide or polypeptide complex of embodiment 134, wherein the single domain antibody is DOM7h-11-15.

Embodiment 143 comprises an isolated polypeptide or polypeptide complex of embodiment 134, wherein the single domain antibody is Alb-1, Alb-8, or Alb-23.

Embodiment 144 comprises an isolated polypeptide or polypeptide complex of embodiment 134, wherein the single domain antibody is 10E.

Embodiment 145 comprises an isolated polypeptide or polypeptide complex of embodiment 134, wherein the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 66, HC-CDR2: SEQ ID NO: 67, and HC-CDR3: SEQ ID NO: 68.

Embodiment 146 comprises an isolated polypeptide or polypeptide complex of embodiment 134, wherein the single domain antibody comprises an amino acid sequence according to SEQ ID NO: 69.

Embodiment 147 comprises an isolated polypeptide or polypeptide complex of embodiment 134, wherein the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 70, HC-CDR2: SEQ ID NO: 71, and HC-CDR3: SEQ ID NO: 72.

Embodiment 148 comprises an isolated polypeptide or polypeptide complex of embodiment 134, wherein the single domain antibody comprises an amino acid sequence according to SEQ ID NO: 73.

Embodiment 149 comprises an isolated polypeptide or polypeptide complex of embodiment 134, wherein the single domain antibody is SA21.

Embodiment 150 comprises an isolated polypeptide or polypeptide complex of any one of embodiments 1-149, wherein the isolated polypeptide or polypeptide complex comprises a modified amino acid, a non-natural amino acid, a modified non-natural amino acid, or a combination thereof.

Embodiment 151 comprises an isolated polypeptide or polypeptide complex of embodiment 150, wherein the modified amino acid or modified non-natural amino acid comprises a post-translational modification.

Embodiment 152 comprises an isolated polypeptide or polypeptide complex of any one of embodiments 1-151, wherein H₁ comprises a linking moiety (L₃) that connects H₁ to P₁.

Embodiment 153 comprises an isolated polypeptide or polypeptide complex of embodiment 152, wherein L₃ is a peptide sequence having at least 5 to no more than 50 amino acids.

Embodiment 154 comprises an isolated polypeptide or polypeptide complex of any one of embodiments 152-153, wherein L₃ is a peptide sequence having at least 10 to no more than 30 amino acids.

Embodiment 155 comprises an isolated polypeptide or polypeptide complex of any one of embodiments 152-154, wherein L₃ is a peptide sequence having at least 10 amino acids.

Embodiment 156 comprises an isolated polypeptide or polypeptide complex of any one of embodiments 152-155, wherein L₃ is a peptide sequence having at least 18 amino acids.

Embodiment 157 comprises an isolated polypeptide or polypeptide complex of any one of embodiments 152-156, wherein L₃ is a peptide sequence having at least 26 amino acids.

Embodiment 158 comprises an isolated polypeptide or polypeptide complex of any one of embodiments 152-157, wherein L₃ has a formula selected from the group consisting of (G₂S)ₙ, (GS)ₙ, (GSGGS)ₙ (SEQ ID NO: 62), (GGGS)ₙ (SEQ ID NO: 63), (GGGGS)ₙ (SEQ ID NO: 64), and (GSSGGS)ₙ (SEQ ID NO: 65), wherein n is an integer of at least 1.

Embodiment 159 comprises an isolated polypeptide or polypeptide complex of embodiment 152, wherein L₃ comprises an amino acid sequence according to SEQ ID NO: 30.

Embodiment 160 comprises an isolated polypeptide or polypeptide complex of embodiment 1, wherein the isolated polypeptide or polypeptide complex comprises an amino acid sequence with at least 95% sequence identity to SEQ ID NOs: 74-132, 241-242.

Embodiment 161 comprises an isolated polypeptide or polypeptide complex of embodiment 1, wherein the isolated polypeptide or polypeptide complex comprises an amino acid sequence with at least 95% sequence identity to SEQ ID NO: 82.

Embodiment 162 comprises an isolated polypeptide or polypeptide complex of embodiment 1, wherein the isolated polypeptide or polypeptide complex comprises an amino acid sequence with at least 95% sequence identity to SEQ ID NO: 83.

Embodiment 163 comprises an isolated polypeptide or polypeptide complex of embodiment 1, wherein the isolated polypeptide or polypeptide complex comprises an amino acid sequence with at least 95% sequence identity to SEQ ID NO: 74 and SEQ ID NO: 75.

Embodiment 164 comprises an isolated polypeptide or polypeptide complex of embodiment 1, wherein the isolated polypeptide or polypeptide complex comprises an amino acid sequence with at least 95% sequence identity to SEQ ID NO: 76 and SEQ ID NO: 77.

Embodiment 165 comprises an isolated polypeptide or polypeptide complex of embodiment 1, wherein the isolated polypeptide or polypeptide complex comprises an amino acid sequence with at least 95% sequence identity to SEQ ID NO: 78 and SEQ ID NO: 79.

Embodiment 166 comprises an isolated polypeptide or polypeptide complex of embodiment 1, wherein the isolated polypeptide or polypeptide complex comprises an amino acid sequence with at least 95% sequence identity to SEQ ID NO: 80 and SEQ ID NO: 81.

Embodiment 167 comprises an isolated polypeptide or polypeptide complex of embodiment 1, wherein the isolated polypeptide or polypeptide complex comprises an amino acid sequence with at least 95% sequence identity to SEQ ID NO: 82 and SEQ ID NO: 83.

Embodiment 168 comprises an isolated polypeptide or polypeptide complex of embodiment 1, wherein the isolated polypeptide or polypeptide complex comprises an amino acid sequence with at least 95% sequence identity to SEQ ID NO: 84 and SEQ ID NO: 85.

Embodiment 169 comprises an isolated polypeptide or polypeptide complex of embodiment 1, wherein the isolated polypeptide or polypeptide complex comprises an amino acid sequence with at least 95% sequence identity to SEQ ID NO: 86 and SEQ ID NO: 87.

Embodiment 170 comprises an isolated polypeptide or polypeptide complex of embodiment 1, wherein the isolated polypeptide or polypeptide complex comprises an amino acid sequence with at least 95% sequence identity to SEQ ID NO: 88 and SEQ ID NO: 89.

Embodiment 171 comprises an isolated polypeptide or polypeptide complex of embodiment 1, wherein the isolated polypeptide or polypeptide complex comprises an amino acid sequence with at least 95% sequence identity to SEQ ID NO: 90 and SEQ ID NO: 91.

Embodiment 172 comprises an isolated polypeptide or polypeptide complex of embodiment 1, wherein the isolated polypeptide or polypeptide complex comprises an amino acid sequence with at least 95% sequence identity to SEQ ID NO: 92 and SEQ ID NO: 93.

Embodiment 173 comprises an isolated polypeptide or polypeptide complex of embodiment 1, wherein the isolated polypeptide or polypeptide complex comprises an amino acid sequence with at least 95% sequence identity to SEQ ID NO: 94 and SEQ ID NO: 95.

Embodiment 174 comprises an isolated polypeptide or polypeptide complex of embodiment 1, wherein the isolated polypeptide or polypeptide complex comprises an amino acid sequence with at least 95% sequence identity to SEQ ID NO: 96 and SEQ ID NO: 97.

Embodiment 175 comprises an isolated polypeptide or polypeptide complex of embodiment 1, wherein the isolated polypeptide or polypeptide complex comprises an amino acid sequence with at least 95% sequence identity to SEQ ID NO: 98 and SEQ ID NO: 99.

Embodiment 176 comprises an isolated polypeptide or polypeptide complex of embodiment 1, wherein the isolated polypeptide or polypeptide complex comprises an amino acid sequence with at least 95% sequence identity to SEQ ID NO: 100 and SEQ ID NO: 101.

Embodiment 177 comprises an isolated polypeptide or polypeptide complex of embodiment 1, wherein the isolated polypeptide or polypeptide complex comprises an amino acid sequence with at least 95% sequence identity to SEQ ID NO: 102 and SEQ ID NO: 103.

Embodiment 178 comprises an isolated polypeptide or polypeptide complex of embodiment 1, wherein the isolated polypeptide or polypeptide complex comprises an amino acid sequence with at least 95% sequence identity to SEQ ID NO: 104 and SEQ ID NO: 105.

Embodiment 179 comprises an isolated polypeptide or polypeptide complex of embodiment 1, wherein the isolated polypeptide or polypeptide complex comprises an amino acid sequence with at least 95% sequence identity to SEQ ID NO: 106 and SEQ ID NO: 107.

Embodiment 180 comprises an isolated polypeptide or polypeptide complex of embodiment 1, wherein the isolated polypeptide or polypeptide complex comprises an amino acid sequence with at least 95% sequence identity to SEQ ID NO: 108 and SEQ ID NO: 109.

Embodiment 181 comprises an isolated polypeptide or polypeptide complex of embodiment 1, wherein the isolated polypeptide or polypeptide complex comprises an amino acid sequence with at least 95% sequence identity to SEQ ID NO: 110 and SEQ ID NO: 111.

Embodiment 182 comprises an isolated polypeptide or polypeptide complex of embodiment 1, wherein the isolated polypeptide or polypeptide complex comprises an amino acid sequence with at least 95% sequence identity to SEQ ID NO: 112 and SEQ ID NO: 113.

Embodiment 183 comprises an isolated polypeptide or polypeptide complex of embodiment 1, wherein the isolated polypeptide or polypeptide complex comprises an amino acid sequence with at least 95% sequence identity to SEQ ID NO: 114 and SEQ ID NO: 115.

Embodiment 184 comprises an isolated polypeptide or polypeptide complex of embodiment 1, wherein the isolated polypeptide or polypeptide complex comprises an amino acid sequence with at least 95% sequence identity to SEQ ID NO: 116 and SEQ ID NO: 117.

Embodiment 185 comprises an isolated polypeptide or polypeptide complex of embodiment 1, wherein the isolated polypeptide or polypeptide complex comprises an amino acid sequence with at least 95% sequence identity to SEQ ID NO: 118 and SEQ ID NO: 119.

Embodiment 186 comprises an isolated polypeptide or polypeptide complex of embodiment 1, wherein the isolated polypeptide or polypeptide complex comprises an amino acid sequence with at least 95% sequence identity to SEQ ID NO: 120 and SEQ ID NO: 121.

Embodiment 187 comprises an isolated polypeptide or polypeptide complex of embodiment 1, wherein the isolated polypeptide or polypeptide complex comprises an amino acid sequence with at least 95% sequence identity to SEQ ID NO: 123 and SEQ ID NO: 124.

Embodiment 188 comprises an isolated polypeptide or polypeptide complex of embodiment 1, wherein the isolated polypeptide or polypeptide complex comprises an amino acid sequence with at least 95% sequence identity to SEQ ID NO: 125 and SEQ ID NO: 126.

Embodiment 189 comprises an isolated polypeptide or polypeptide complex of embodiment 1, wherein the isolated polypeptide or polypeptide complex comprises an amino acid sequence with at least 95% sequence identity to SEQ ID NO: 127 and SEQ ID NO: 128.

Embodiment 190 comprises an isolated polypeptide or polypeptide complex of embodiment 1, wherein the isolated polypeptide or polypeptide complex comprises an amino acid sequence with at least 95% sequence identity to SEQ ID NO: 129 and SEQ ID NO: 130.

Embodiment 191 comprises an isolated polypeptide or polypeptide complex of embodiment 1, wherein the isolated polypeptide or polypeptide complex comprises an amino acid sequence with at least 95% sequence identity to SEQ ID NO: 241 and SEQ ID NO: 131.

Embodiment 192 comprises an isolated polypeptide or polypeptide complex of embodiment 1, wherein the isolated polypeptide or polypeptide complex comprises an amino acid sequence with at least 95% sequence identity to SEQ ID NO: 132 and SEQ ID NO: 242.

Embodiment 193 comprises a pharmaceutical composition comprising: (a) the isolated polypeptide or polypeptide complex of any one of embodiments 1-193; and (b) a pharmaceutically acceptable excipient.

Embodiment 194 comprises an isolated recombinant nucleic acid molecule encoding the isolated polypeptide or polypeptide complex of any one of embodiments 1-Error! Reference source not found.

Embodiment 195 comprises an isolated polypeptide or polypeptide complex according to Formula II: L₁ₐ-P₁ₐ-H₁a wherein: L₁ₐ comprises a tumor specific protease-cleaved linking moiety that when uncleaved connects P₁ₐ to a first antigen recognizing molecule that binds to an effector cell antigen and the first antigen recognizing molecule is connected to a second antigen recognizing molecule that binds to TROP2; P₁ₐ comprises a peptide that binds to the first antigen recognizing molecule when L₁ₐ is uncleaved; and H₁ₐ comprises a half-life extending molecule.

Embodiment 196 comprises an isolated polypeptide or polypeptide complex of embodiment 195, wherein P₁ₐ when L₁ₐ is uncleaved impairs binding of the first antigen recognizing molecule to the effector cell antigen.

Embodiment 197 comprises an isolated polypeptide or polypeptide complex of any one of embodiments 195-196, wherein the first antigen recognizing molecule comprises an antibody or antibody fragment.

Embodiment 198 comprises an isolated polypeptide or polypeptide complex of any one of embodiments 195-197, wherein the effector cell antigen is an anti-CD3 effector cell antigen.

Embodiment 199 comprises an isolated polypeptide or polypeptide complex of any one of embodiments 195-198, wherein P₁ₐ has less than 70% sequence homology to the effector cell antigen.

Embodiment 200 comprises an isolated polypeptide or polypeptide complex of any one of embodiments 195-199, wherein P₁ₐ comprises a peptide sequence of at least 10 amino acids in length.

Embodiment 201 comprises an isolated polypeptide or polypeptide complex of any one of embodiments 195-200, wherein P₁ₐ comprises a peptide sequence of at least 10 amino acids in length and no more than 20 amino acids in length.

Embodiment 202 comprises an isolated polypeptide or polypeptide complex of any one of embodiments 195-201, wherein P₁ₐ comprises a peptide sequence of at least 16 amino acids in length.

Embodiment 203 comprises an isolated polypeptide or polypeptide complex of any one of embodiments 195-200 and 202, wherein P₁ₐ comprises a peptide sequence of no more than 40 amino acids in length.

Embodiment 204 comprises an isolated polypeptide or polypeptide complex of any one of embodiments 195-203, wherein P₁ₐ comprises at least two cysteine amino acid residues.

Embodiment 205 comprises an isolated polypeptide or polypeptide complex of any one of embodiments 195-204, wherein P₁ₐ comprises a cyclic peptide or a linear peptide.

Embodiment 206 comprises an isolated polypeptide or polypeptide complex of any one of embodiments 195-205, wherein P₁ₐ comprises a cyclic peptide.

Embodiment 207 comprises an isolated polypeptide or polypeptide complex of any one of embodiments 195-205, wherein P₁ₐ comprises a linear peptide.

Embodiment 208 comprises an isolated polypeptide or polypeptide complex of any one of embodiments 195-201 and 203-207, wherein P₁ₐ comprises an amino acid sequence selected from the group consisting of SEQ ID NO: 26, 27, or 122.

Embodiment 209 comprises an isolated polypeptide or polypeptide complex of any one of embodiments 195-201 and 203-207, wherein P₁ₐ comprises an amino acid sequence according to Z₁-Z₂-C-Z₄-P-Z₆-Z₇-Z₈-Z₉-Z₁₀-Z₁₁-Z₁₂-C-Z₁₄ and Z₁ is selected from D, Y, F, I, N, V, H, L, A, T, S, and P; Z₂ is selected from D, Y, L, F, I, N, A, V, H, T, and S; Z₄ is selected from G and W; Z₆ is selected from E, D, V, and P; Z₇ is selected from W, L, F, V, G, M, I, and Y; Z₈ is selected from E, D, P, and Q; Z₉ is selected from E, D, Y, V, F, W, P, L, and Q; Z₁₀ is selected from S, D, Y, T, I, F, V, N, A, P, L, and H; Z₁₁ is selected from I, Y, F, V, L, T, N, S, D, A, and H; Z₁₂ is selected from F, D, Y, L, I, V, A, N, T, P, S, and H; Z₁₄ is selected from D, Y, N, F, I, P, V, A, T, H, L and S; or P₁ₐ comprises an amino acid sequence according to U₁-U₂-C-U₄-P-U₆-U₇-U₈-U₉-U₁₀-U₁₁-U₁₂-C-U₁₄ and U₁ is selected from D, Y, F, I, N, V, H, L, A, T, S, and P; U₂ is selected from D, Y, L, F, I, N, A, V, H, T, and S; U₄ is selected from G and W; U₆ is selected from E, D, V, and P; U₇ is selected from W, L, F, V, G, M, I, and Y; U₈ is selected from E, D, P, and Q; U₉ is selected from E, D, Y, V, F, W, P, L, and Q; U₁₀ is selected from S, D, Y, T, I, F, V, N, A, P, L, and H; U₁₁ is selected from I, Y, F, V, L, T, N, S, D, A, and H; U₁₂ is selected from F, D, Y, L, I, V, A, N, T, P, S, G, and H; U₁₄ is selected from D, Y, N, F, I, P, V, A, T, H, L, M, and S.

Embodiment 210 comprises an isolated polypeptide or polypeptide complex of embodiment 209, wherein Z₁ is selected from D, Y, F, I, and N; Z₂ is selected from D, Y, L, F, I, and N; Z₄ is selected from G and W; Z₆ is selected from E and D; Z₇ is selected from W, L, F, and V; Z₈ is selected from E and D; Z₉ is selected from E, D, Y, and V; Z₁₀ is selected from S, D, Y, T, and I; Z₁₁ is selected from I, Y, F, V, L, and T; Z₁₂ is selected from F, D, Y, L, I, V, A, and N; Z₁₄ is selected from D, Y, N, F, I, and P; and U₁ is selected from D, Y, F, I, V, and N; U₂ is selected from D, Y, L, F, I, and N; U₄ is selected from G and W; U₆ is selected from E and D; U₇ is selected from W, L, F, G, and V; U₈ is selected from E and D; U₉ is selected from E, D, Y, and V; U₁₀ is selected from S, D, Y, T, and I; U₁₁ is selected from I, Y, F, V, L, and T; U₁₂ is selected from F, D, Y, L, I, V, A, G, and N; U₁₄ is selected from D, Y, N, F, I, M, and P.

Embodiment 211 comprises an isolated polypeptide or polypeptide complex of any one of embodiments 209-210, wherein Z₁ is selected from D, Y, and F; Z₂ is selected from D, Y, L, and F; Z₄ is selected from G and W; Z₆ is selected from E and D; Z₇ is selected from W, L, and F; Z₈ is selected from E and D; Z₉ is selected from E and D; Z₁₀ is selected from S, D, and Y; Z₁ is selected from I, Y, and F; Z₁₂ is selected from F, D, Y, and L; Z₁₄ is selected from D, Y, and N; and U₁ is selected D, Y, V, and F; U₂ is selected from D, Y, L, and F; U₄ is selected from G and W; U₆ is selected from E and D; U₇ is selected from W, L, G, and F; U₈ is selected from E and D; U₉ is selected from E and D; U₁₀ is selected from S, D, T, and Y; U₁₁ is selected from I, Y, V, L, and F; U₁₂ is selected from F, D, Y, G, A, and L; U₁₄ is selected from D, Y, M, and N.

Embodiment 212 comprises an isolated polypeptide or polypeptide complex of any one of embodiments 195-201 and 203-207, wherein P₁ₐ comprises the amino acid sequences according to SEQ ID NOs: 202-228.

Embodiment 213 comprises an isolated polypeptide or polypeptide complex of any one of embodiments 195-201 and 203-207, wherein P₁ₐ comprises an amino acid sequence according to any of the sequences of Table 35.

Embodiment 214 comprises an isolated polypeptide or polypeptide complex of any one of embodiments 209-211 or 213, wherein P₁ₐ comprises the amino acid sequences according to SEQ ID NOs: 229-240.

Embodiment 215 comprises an isolated polypeptide or polypeptide complex of any one of embodiments 209-211 or 213, wherein P₁ₐ comprises the amino acid sequence according to SEQ ID NO: 239 or a peptide sequence that has 1, 2, or 3, amino acid substitutions, additions, or deletions relative to the amino acid sequence of SEQ ID NO: 239.

Embodiment 216 comprises an isolated polypeptide or polypeptide complex of any one of embodiments 209-211 or 213, wherein P₁ₐ comprises the amino acid sequence according to SEQ ID NO: 27 or a peptide sequence that has 1, 2, or 3, amino acid substitutions, additions, or deletions relative to the amino acid sequence of SEQ ID NO: 27.

Embodiment 216 comprises an isolated polypeptide or polypeptide complex of any one of embodiments 195-201 and 203-207 wherein P₁ₐ comprises the amino acid sequence according to SEQ ID NO: 26 or a peptide sequence that has 1, 2, or 3, amino acid substitutions, additions, or deletions relative to the amino acid sequence of SEQ ID NO: 26.

Embodiment 218 comprises an isolated polypeptide or polypeptide complex of any one of embodiments 209-211 or 213, wherein P₁ₐ comprises the amino acid sequence according to SEQ ID NO: 239.

Embodiment 219 comprises an isolated polypeptide or polypeptide complex of any one of embodiments 209-211 or 213, wherein P₁ₐ comprises the amino acid sequence according to SEQ ID NO: 27.

Embodiment 220 comprises an isolated polypeptide or polypeptide complex of any one of embodiments 195-201 and 203-207, wherein P₁ₐ comprises the amino acid sequence according to SEQ ID NO: 26.

Embodiment 221 comprises an isolated polypeptide or polypeptide complex of any one of embodiments 195-220, wherein H₁ₐ comprises a polymer.

Embodiment 222 comprises an isolated polypeptide or polypeptide complex of embodiment 221, wherein the polymer is polyethylene glycol (PEG).

Embodiment 223 comprises an isolated polypeptide or polypeptide complex of any one of embodiments 195-221, wherein H₁ₐ comprises albumin.

Embodiment 224 comprises an isolated polypeptide or polypeptide complex of any one of embodiments 195-221, wherein H₁ₐ comprises an Fc domain.

Embodiment 225 comprises an isolated polypeptide or polypeptide complex of embodiment 223, wherein the albumin is serum albumin.

Embodiment 226 comprises an isolated polypeptide or polypeptide complex of embodiment 223, wherein the albumin is human serum albumin.

Embodiment 227 comprises an isolated polypeptide or polypeptide complex of any one of embodiments 195-220, wherein H₁ₐ comprises a polypeptide, a ligand, or a small molecule.

Embodiment 228 comprises an isolated polypeptide or polypeptide complex of embodiment 227, wherein the polypeptide, the ligand or the small molecule binds a serum protein or a fragment thereof, a circulating immunoglobulin or a fragment thereof, or CD35/CR1.

Embodiment 229 comprises an isolated polypeptide or polypeptide complex of embodiment 228, wherein the serum protein comprises a thyroxine-binding protein, a transthyretin, a 1-acid glycoprotein, a transferrin, transferrin receptor or a transferrin-binding portion thereof, a fibrinogen, or an albumin.

Embodiment 230 comprises an isolated polypeptide or polypeptide complex of embodiment 228, wherein the circulating immunoglobulin molecule comprises IgG1, IgG2, IgG3, IgG4, slgA, IgM or IgD.

Embodiment 231 comprises an isolated polypeptide or polypeptide complex of any one of embodiments 228-229, wherein the serum protein is albumin.

Embodiment 232 comprises an isolated polypeptide or polypeptide complex of any one of embodiments 227-231, wherein the polypeptide is an antibody.

Embodiment 233 comprises an isolated polypeptide or polypeptide complex of embodiment 232, wherein the antibody comprises a single domain antibody, a single chain variable fragment or a Fab.

Embodiment 234 comprises an isolated polypeptide or polypeptide complex of any one of embodiments 232-233, wherein the antibody comprises a single domain antibody that binds to albumin.

Embodiment 235 comprises an isolated polypeptide or polypeptide complex of any one of embodiments 232-234, wherein the antibody is a human or humanized antibody.

Embodiment 236 comprises an isolated polypeptide or polypeptide complex of embodiment 233, wherein the single domain antibody is 645gH1gL1.

Embodiment 237 comprises an isolated polypeptide or polypeptide complex of embodiment 233, wherein the single domain antibody is 645dsgH5gL4.

Embodiment 238 comprises an isolated polypeptide or polypeptide complex of embodiment 233, wherein the single domain antibody is 23-13-A01-sc02.

Embodiment 239 comprises an isolated polypeptide or polypeptide complex of embodiment 233, wherein the single domain antibody is A10m3 or a fragment thereof.

Embodiment 240 comprises an isolated polypeptide or polypeptide complex of embodiment 233, wherein the single domain antibody is DOM7r-31.

Embodiment 241 comprises an isolated polypeptide or polypeptide complex of embodiment 233, wherein the single domain antibody is DOM7h-11-15.

Embodiment 242 comprises an isolated polypeptide or polypeptide complex of embodiment 233, wherein the single domain antibody is Alb-1, Alb-8, or Alb-23.

Embodiment 243 comprises an isolated polypeptide or polypeptide complex of embodiment 233, wherein the single domain antibody is 10E.

Embodiment 244 comprises an isolated polypeptide or polypeptide complex of embodiment 233, wherein the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 66, HC-CDR2: SEQ ID NO: 67, and HC-CDR3: SEQ ID NO: 68.

Embodiment 245 comprises an isolated polypeptide or polypeptide complex of embodiment 233, wherein the single domain antibody comprises an amino acid sequence according to SEQ ID NO: 69.

Embodiment 246 comprises an isolated polypeptide or polypeptide complex of embodiment 233, wherein the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 70, HC-CDR2: SEQ ID NO: 71, and HC-CDR3: SEQ ID NO: 72.

Embodiment 247 comprises an isolated polypeptide or polypeptide complex of embodiment 233, wherein the single domain antibody comprises an amino acid sequence according to SEQ ID NO: 73.

Embodiment 248 comprises an isolated polypeptide or polypeptide complex of embodiment 233, wherein the single domain antibody is SA21.

Embodiment 249 comprises an isolated polypeptide or polypeptide complex of any one of embodiments 195-248, wherein H₁ₐ comprises a linking moiety (L₁ₐ) that connects H₁ₐ to Pia.

Embodiment 250 comprises an isolated polypeptide or polypeptide complex of embodiment 249, wherein L₁ₐ is a peptide sequence having at least 5 to no more than 50 amino acids.

Embodiment 251 comprises an isolated polypeptide or polypeptide complex of any one of embodiments 249-250, wherein L₁ₐ is a peptide sequence having at least 10 to no more than 30 amino acids.

Embodiment 252 comprises an isolated polypeptide or polypeptide complex of any one of embodiments 249-251, wherein L₁ₐ is a peptide sequence having at least 10 amino acids.

Embodiment 253 comprises an isolated polypeptide or polypeptide complex of any one of embodiments 249-252, wherein L₁ₐ is a peptide sequence having at least 18 amino acids.

Embodiment 254 comprises an isolated polypeptide or polypeptide complex of any one of embodiments 249-253, wherein L₁ₐ is a peptide sequence having at least 26 amino acids.

Embodiment 255 comprises an isolated polypeptide or polypeptide complex of any one of embodiments 249-254, wherein L₁ₐ has a formula selected from the group consisting of (G₂S)ₙ, (GS)ₙ, (GSGGS)ₙ (SEQ ID NO: 62), (GGGS)ₙ (SEQ ID NO: 63), (GGGGS)ₙ (SEQ ID NO: 64), and (GSSGGS)ₙ (SEQ ID NO: 65), wherein n is an integer of at least 1.

Embodiment 256 comprises an isolated polypeptide or polypeptide complex of embodiment 249, wherein L₁ₐ comprises an amino acid sequence according to any one of SEQ ID NOs: 28-61.

Embodiment 257 comprises a polypeptide complex comprising a structural arrangement according to Configuration 1:

wherein the polypeptide complex comprises a single chain variable fragment (scFv) comprising a light chain variable domain and a heavy chain variable domain, wherein the scFv further comprises a peptide that impairs binding of the scFv to an effector cell antigen and the peptide is linked to a N-terminus of the heavy chain variable domain of the scFv with a linking moiety that is a substrate for a tumor specific protease, and the peptide further comprises a half-life extending molecule; and a Fab that binds to TROP2, wherein the Fab comprises a Fab light chain polypeptide and a Fab heavy chain polypeptide, wherein the Fab heavy chain polypeptide is linked to a C terminus of the light chain variable domain of the scFv, and wherein the Fab further comprises P₂ and L₂, wherein P₂ comprises a peptide that impairs binding to TROP2; and L₂ comprises a linking moiety that connects the Fab light chain polypeptide to P₂ and is a substrate for a tumor specific protease.

Embodiment 258 comprises a polypeptide complex comprising a structural arrangement according to Configuration 2:

wherein the polypeptide complex comprises a single chain variable fragment (scFv) comprising a light chain variable domain and a heavy chain variable domain, wherein the scFv further comprises a peptide that impairs binding of the scFv to an effector cell antigen and the peptide is linked to a N-terminus of the heavy chain variable domain of the scFv with a linking moiety that is a substrate for a tumor specific protease, and the peptide further comprises a half-life extending molecule; and a Fab that binds to TROP2, wherein the Fab comprises a Fab light chain polypeptide and a Fab heavy chain polypeptide, wherein the Fab light chain polypeptide is linked to a C terminus of the light chain variable domain of the scFv, and wherein the Fab further comprises P₂ and L₂, wherein P₂ comprises a peptide that impairs binding to TROP2; and L₂ comprises a linking moiety that connects the Fab heavy chain polypeptide to P₂ and is a substrate for a tumor specific protease.

Embodiment 259 comprises an isolated polypeptide or polypeptide complex comprising an anti-TROP2 binding domain that is linked to a peptide that impairs binding of the anti-TROP2 binding to TROP2 wherein the peptide comprises an amino acid sequence according to X₁-X₂-X₃-X₄-C-X₆-X₇-X₈-X₉-X₁₀-C-X₁₂-X₁₃-X₁₄ and X₁ is selected from N, D, S, Y, A, F, H, T, L, and V; X₂ is selected from S, T, D, A, H, V, Y, N, F, I, and L; X₃ is selected from L, I, and V; X₄ is selected from F, L, V, M, W, I, Y, and H; X₆ is selected from V, F, L, I, and W; X₇ is selected from K, R, Q, N, H, and M; X₈ is selected from N and K; X₉ is selected from L, V, and I; X₁₀ is selected from Y, W, F, Q, and L; X₁₂ is selected from W and V; X₁₃ is selected from I, N, H, T, V, Y, and D; X₁₄ is selected from D, V, A, S, I, T, N, Y, H, and P; or the peptide comprises an amino acid according to J₁-J₂-J₃-C-J₅-J₆-J₇-J₈-W-J₁₀-J₁₁-C-J₁₃-J₁₄ and J₁ is selected from V, I, L, P, E, F, and M; J₂ is selected from D and N; J₃ is selected from F and W; J₅ is selected from A, E, S, R, K, Y, L, Q, G, M, F, T, W, and D; J₆ is selected from L, M, I, V, F, T, R, and S; J₇ is selected from Y, F, and N; J₈ is selected from N, R, D, H, K, Q, S, G, A, E, and M; J₁₀ is selected from P and R; J₁₁ is selected from V and I; J₁₃ is selected from D, G, N, R, S, Q, Y, T, A, E, L, V, K, M, I, H, F, and W; J₁₄ is selected from T, S, Q, L, D, N, A, E, K, M, V, R, I, H, P, V, and W; or the peptide comprises an amino acid sequence according to B₁-B₂-B₃-C-B₅-B₆-B₇-Bg-W-B₁₀-B₁₁-C-B₁₃-B₁₄ and B₁ is selected from V, I, L, P, E, F, and M; B₂ is selected from D and N; B₃ is selected from F and W; B₅ is selected from A, E, S, R, K, Y, L, Q, G, M, F, T, W, and D; B₆ is selected from L, M, I, V, F, T, R, and S; B₇ is selected from Y, F, and N; B₈ is selected from N, R, D, H, K, Q, S, G, A, E, and M; B₁₀ is selected from P and R; B₁₁ is selected from V and I; B₁₃ is selected from D, G, N, R, S, Q, Y, T, A, E, L, V, K, M, I, H, F, and W; B₁₄ is selected from T, S, Q, L, D, N, A, E, K, M, V, R, I, H, P, V, G, and W.

Embodiment 260 comprises an isolated polypeptide or polypeptide complex of embodiment 259, wherein X₁ is selected from N, D, S, Y, A, F, and T; X₂ is selected from S, T, D, A, H, V, Y, and N; X₃ is L; X₄ is selected from F, L, V, M, and W; X₆ is selected from V, F, and L; X₇ is selected from K, R, Q, and N; X₈ is selected from N and K; X₉ is selected from L, V, and I; X₁₀ is selected from Y, W, and F; X₁₂ is W; X₁₃ is selected from I, N, H, and T; X₁₄ is selected from D, V, A, S, I, T, and N.

Embodiment 261 comprises an isolated polypeptide or polypeptide complex of any one of embodiments 259-260, wherein X₁ is selected from N, D, S, and Y; X₂ is selected from S, T, and D; X₃ is L; X₄ is selected from F, L, and V; X₆ is selected from V and F; X₇ is selected from K, R, and Q; X₈ is N; X₉ is selected from L and V; X₁₀ is selected from Y and W; X₁₂ is W; X₁₃ is selected from I, N, and H; X₁₄ is selected from D, V, A, and S.

Embodiment 262 comprises an isolated polypeptide or polypeptide complex of any one of embodiments 259-261, wherein J₁ is selected V, I, and L; J₂ is D; J₃ is F; J₈ is selected from A, E, S, R, K, Y, and L; J₆ is selected from L, M, and I; J₇ is Y; J₈ is selected from N, R, D, H, K, Q, S, and G; J₁₀ is P; J₁₁ is selected from V and I; J₁₃ is selected from D, G, N, R, S, Q, Y, T, and A; J₁₄ is selected from T, S, Q, L, D, N, A, and E.

Embodiment 263 comprises an isolated polypeptide or polypeptide complex of any one of embodiments 259-262, wherein J₁ is selected from V, I, and L; J₂ is D; J₃ is F; J₈ is selected from A, E, S, and R; J₆ is selected from L, M, and I; J₇ is Y; J₈ is selected from N, R, and D; J₁₀ is P; J₁₁ is selected from V and I; J₁₃ is selected from D, G, N, R, S, and Q; J₁₄ is selected from T, S, Q, and L.

Embodiment 264 comprises an isolated polypeptide or polypeptide complex of embodiment 259, wherein B₁ is selected from V, I, and L; B₂ is D; B₃ is F; B₅ is selected from A, E, S, R, K, Y, M, G, and L; B₆ is selected from L, M, and I; B₇ is Y; B₈ is selected from N, R, D, H, K, Q, S, and G; B₁₀ is P; B₁₁ is selected from V and I; B₁₃ is selected from D, G, N, R, S, Q, Y, T, H, and A; B₁₄ is selected from T, S, Q, L, D, N, A, G, and E.

Embodiment 265 comprises an isolated polypeptide or polypeptide complex of embodiment 264, wherein B₁ is selected from V, I, and L; B₂ is D; B₃ is F; B₅ is selected from A, E, S, K, M, G, and R; B₆ is selected from L, M, and I; B₇ is Y; B₈ is selected from N, R, S, H, and D; B₁₀ is P; B₁₁ is selected from V and I; B₁₃ is selected from D, G, N, R, S, H, A, Y, and Q; B₁₄ is selected from T, S, Q, G, and L.

Embodiment 266 comprises an isolated polypeptide or polypeptide complex of embodiment 259, wherein the peptide comprises an amino acid sequence according to any of the sequences of Table 27.

Embodiment 267 comprises an isolated polypeptide or polypeptide complex of any one of embodiments 259-261, or 266, wherein the peptide comprises the amino acid sequences according to SEQ ID NOs: 159-178.

Embodiment 268 comprises an isolated polypeptide or polypeptide complex of embodiment 259, wherein the peptide comprises an amino acid sequences according to any of the sequences of Table 29.

Embodiment 269 comprises an isolated polypeptide or polypeptide complex of any one of embodiments 259, 262-265, wherein the peptide comprises the amino acid sequences according to SEQ ID NOs: 179-201.

Embodiment 270 comprises an isolated polypeptide or polypeptide complex of any one of embodiments 259, 264-265, wherein the peptide comprises the amino acid sequence according to SEQ ID NO: 24 or a peptide sequence that has 1, 2, or 3, amino acid substitutions, additions, or deletions relative to the amino acid sequence of SEQ ID NO: 24.

Embodiment 271 comprises an isolated polypeptide or polypeptide complex of any one of embodiments 259, 264-265, wherein the peptide comprises the amino acid sequence according to SEQ ID NO: 181 or a peptide sequence that has 1, 2, or 3, amino acid substitutions, additions, or deletions relative to the amino acid sequence of SEQ ID NO: 181.

Embodiment 272 comprises an isolated polypeptide or polypeptide complex of any one of embodiments 259, 264-265, wherein the peptide comprises the amino acid sequence according to SEQ ID NO: 186 or a peptide sequence that has 1, 2, or 3, amino acid substitutions, additions, or deletions relative to the amino acid sequence of SEQ ID NO: 186.

Embodiment 273 comprises an isolated polypeptide or polypeptide complex of any one of embodiments 259, 264-265, wherein the peptide comprises the amino acid sequence according to SEQ ID NO: 24.

Embodiment 274 comprises an isolated polypeptide or polypeptide complex of any one of embodiments 259, 264-265, wherein the peptide comprises the amino acid sequence according to SEQ ID NO: 181.

Embodiment 275 comprises an isolated polypeptide or polypeptide complex of any one of embodiments 259, 264-265, wherein the peptide comprises the amino acid sequence according to SEQ ID NO: 186.

Embodiment 276 comprises an isolated polypeptide or polypeptide complex of any one of embodiments 259-275, wherein the anti-TROP2 binding domain comprises an antibody or an antibody fragment.

Embodiment 277 comprises an isolated polypeptide or polypeptide complex of embodiment 276, wherein the antibody or antibody fragment comprises a single chain variable fragment, a single domain antibody, Fab, or Fab′.

Embodiment 278 comprises an isolated polypeptide or polypeptide complex of any one of embodiments 276-277, wherein the anti-TROP2 binding domain comprises heavy chain complementarity determining regions HC-CDR1, HC-CDR2, and HC-CDR3, wherein the HC-CDR1, the HC-CDR2, and the HC-CDR3 comprise: HC-CDR1: SEQ ID NO: 15, HC-CDR2: SEQ ID NO: 16, and HC-CDR3: SEQ ID NO: 17; and the anti-TROP2 binding domain comprises light chain complementarity determining regions CDRs: LC-CDR1, LC-CDR2, and LC-CDR3, wherein the LC-CDR1, the LC-CDR2, and the LC-CDR3 of the Fab comprise LC-CDR1: SEQ ID NO: 18, LC-CDR2: SEQ ID NO:19, and LC-CDR3: SEQ ID NO: 20.

Embodiment 279 comprises an isolated polypeptide or polypeptide complex of embodiment 277, wherein the antibody or antibody fragment comprises the Fab.

Embodiment 280 comprises an isolated polypeptide or polypeptide complex of any one of embodiments 259-279, wherein the anti-TROP2 binding domain comprises amino acid sequences according to SEQ ID NOs: 21-22.

Embodiment 281 comprises an isolated polypeptide or polypeptide complex of embodiment 280, wherein the antibody or antibody fragment comprises the Fab or Fab′.

Embodiment 282 comprises an isolated polypeptide or polypeptide complex of any one of embodiments 259-280, wherein the isolated polypeptide or polypeptide complex further comprises a half-life extending molecule (H₁).

Embodiment 283 comprises an isolated polypeptide or polypeptide complex of embodiment 282, wherein the half-life extending molecule is linked to the peptide.

Embodiment 284 comprises an isolated polypeptide or polypeptide complex of any one of embodiments 282-283, wherein H₁ comprises a polymer.

Embodiment 285 comprises an isolated polypeptide or polypeptide complex of embodiment 284, wherein the polymer is polyethylene glycol (PEG).

Embodiment 286 comprises an isolated polypeptide or polypeptide complex of any one of embodiments 282-284, wherein H₁ comprises albumin.

Embodiment 287 comprises an isolated polypeptide or polypeptide complex of any one of embodiments 282-284, wherein H₁ comprises an Fc domain.

Embodiment 288 comprises an isolated polypeptide or polypeptide complex of embodiment 286, wherein the albumin is serum albumin.

Embodiment 289 comprises an isolated polypeptide or polypeptide complex of embodiment 286, wherein the albumin is human serum albumin.

Embodiment 290 comprises an isolated polypeptide or polypeptide complex of any one of embodiments 282-283, wherein H₁ comprises a polypeptide, a ligand, or a small molecule.

Embodiment 291 comprises an isolated polypeptide or polypeptide complex of embodiment 290, wherein the polypeptide, the ligand or the small molecule binds serum protein or a fragment thereof, a circulating immunoglobulin or a fragment thereof, or CD35/CR1.

Embodiment 292 comprises an isolated polypeptide or polypeptide complex of embodiment 291, wherein the serum protein comprises a thyroxine-binding protein, a transthyretin, a 1-acid glycoprotein, a transferrin, transferrin receptor or a transferrin-binding portion thereof, a fibrinogen, or an albumin.

Embodiment 293 comprises an isolated polypeptide or polypeptide complex of embodiment 291, wherein the circulating immunoglobulin molecule comprises IgG1, IgG2, IgG3, IgG4, slgA, IgM or IgD.

Embodiment 294 comprises an isolated polypeptide or polypeptide complex of any one of embodiments 291-292, wherein the serum protein is albumin.

Embodiment 295 comprises an isolated polypeptide or polypeptide complex of any one of embodiments 290-294, wherein the polypeptide is an antibody.

Embodiment 296 comprises an isolated polypeptide or polypeptide complex of embodiment 295, wherein the antibody comprises a single domain antibody, a single chain variable fragment, or a Fab.

Embodiment 297 comprises an isolated polypeptide or polypeptide complex of embodiment 296, wherein the single domain antibody comprises a single domain antibody that binds to albumin.

Embodiment 298 comprises an isolated polypeptide or polypeptide complex of any one of embodiments 296-297, wherein the single domain antibody is a human or humanized antibody.

Embodiment 299 comprises an isolated polypeptide or polypeptide complex of embodiment 296, wherein the single domain antibody is 645gH1gL1.

Embodiment 300 comprises an isolated polypeptide or polypeptide complex of embodiment 296, wherein the single domain antibody is 645dsgH5gL4.

Embodiment 301 comprises an isolated polypeptide or polypeptide complex of embodiment 296, wherein the single domain antibody is 23-13-A01-sc02.

Embodiment 302 comprises an isolated polypeptide or polypeptide complex of embodiment 296, wherein the single domain antibody is A10m3 or a fragment thereof.

Embodiment 303 comprises an isolated polypeptide or polypeptide complex of embodiment 296, wherein the single domain antibody is DOM7r-31.

Embodiment 304 comprises an isolated polypeptide or polypeptide complex of embodiment 296, wherein the single domain antibody is DOM7h-11-15.

Embodiment 305 comprises an isolated polypeptide or polypeptide complex of embodiment 296, wherein the single domain antibody is Alb-1, Alb-8, or Alb-23.

Embodiment 306 comprises an isolated polypeptide or polypeptide complex of embodiment 296, wherein the single domain antibody is 10E.

Embodiment 307 comprises an isolated polypeptide or polypeptide complex of embodiment 296, wherein the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 66, HC-CDR2: SEQ ID NO: 67, and HC-CDR3: SEQ ID NO: 68.

Embodiment 308 comprises an isolated polypeptide or polypeptide complex of embodiment 296, wherein the single domain antibody comprises an amino acid sequence according to SEQ ID NO: 69.

Embodiment 309 comprises an isolated polypeptide or polypeptide complex of embodiment 296, wherein the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 70, HC-CDR2: SEQ ID NO: 71, and HC-CDR3: SEQ ID NO: 72.

Embodiment 310 comprises an isolated polypeptide or polypeptide complex of embodiment 296, wherein the single domain antibody comprises an amino acid sequence according to SEQ ID NO: 73.

Embodiment 311 comprises an isolated polypeptide or polypeptide complex of embodiment 296, wherein the single domain antibody is SA21.

Embodiment 312 comprises an isolated polypeptide or polypeptide complex of any one of embodiments 259-311, wherein the isolated polypeptide or polypeptide complex comprises a modified amino acid, a non-natural amino acid, a modified non-natural amino acid, or a combination thereof.

Embodiment 313 comprises an isolated polypeptide or polypeptide complex of embodiment 312, wherein the modified amino acid or modified non-natural amino acid comprises a post-translational modification.

Embodiment 314 comprises an isolated polypeptide or polypeptide complex of any one of embodiments 282-313, wherein H₁ comprises a linking moiety (L₃) that connects H₁ to the peptide.

Embodiment 315 comprises an isolated polypeptide or polypeptide complex of embodiment 314, wherein L₃ is a peptide sequence having at least 5 to no more than 50 amino acids.

Embodiment 316 comprises an isolated polypeptide or polypeptide complex of any one of embodiments 314-315, wherein L₃ is a peptide sequence having at least 10 to no more than 30 amino acids.

Embodiment 317 comprises an isolated polypeptide or polypeptide complex of any one of embodiments 314-315, wherein L₃ is a peptide sequence having at least 10 amino acids.

Embodiment 318 comprises an isolated polypeptide or polypeptide complex of any one of embodiments 314-317, wherein L₃ is a peptide sequence having at least 18 amino acids.

Embodiment 319 comprises an isolated polypeptide or polypeptide complex of any one of embodiments 314-318, wherein L₃ is a peptide sequence having at least 26 amino acids.

Embodiment 320 comprises an isolated polypeptide or polypeptide complex of any one of embodiments 314-319, wherein L₃ has a formula selected from the group consisting of (G₂S)ₙ, (GS)ₙ, (GSGGS)ₙ (SEQ ID NO: 62), (GGGS)ₙ (SEQ ID NO: 63), (GGGGS)ₙ (SEQ ID NO: 64), and (GSSGGS) (SEQ ID NO: 65), wherein n is an integer of at least 1.

Embodiment 321 comprises an isolated polypeptide or polypeptide complex of any one of embodiments 314-315, wherein L₃ comprises an amino acid sequence according to SEQ ID NO: 30.

Embodiment 322 comprises a method of treating triple-negative breast cancer comprising administering to a subject in need thereof an isolated polypeptide or polypeptide complex according to Embodiments 1-192, or 257-321.

Embodiment 323 comprises a method of treating urothelial cancer comprising administering to a subject in need thereof an isolated polypeptide or polypeptide complex according to Embodiments 1-192, or 257-321.

Embodiment 324 comprises a method of treating non-small cell lung cancer (NSCLC) comprising administering to a subject in need thereof an isolated polypeptide or polypeptide complex according to Embodiments 1-192, or 257-321.

Embodiment 325 comprises a method of treating small cell lung cancer (SCLC) comprising administering to a subject in need thereof an isolated polypeptide or polypeptide complex according to Embodiments 1-192, or 257-321.

Embodiment 326 comprises a method of treating gastric cancer comprising administering to a subject in need thereof an isolated polypeptide or polypeptide complex according to Embodiments 1-192, or 257-321.

Embodiment 327 comprises a method of treating esophageal cancer comprising administering to a subject in need thereof an isolated polypeptide or polypeptide complex according to Embodiments 1-192, or 257-321.

Embodiment 328 comprises a method of treating head and neck cancer comprising administering to a subject in need thereof an isolated polypeptide or polypeptide complex according to Embodiments 1-192, or 257-321.

Embodiment 329 comprises a method of treating prostate cancer comprising administering to a subject in need thereof an isolated polypeptide or polypeptide complex according to Embodiments 1-192, or 257-321.

Embodiment 330 comprises a method of treating endometrial cancer comprising administering to a subject in need thereof an isolated polypeptide or polypeptide complex according to Embodiments 1-192, or 257-321.

Embodiment 331 comprises a method of treating breast cancer comprising administering to a subject in need thereof an isolated polypeptide or polypeptide complex according to Embodiments 1-192, or 257-321.

Embodiment 332 comprises a method of treating colon cancer comprising administering to a subject in need thereof an isolated polypeptide or polypeptide complex according to Embodiments 1-192, or 257-321.

Embodiment 333 comprises a method of treating glioma comprising administering to a subject in need thereof an isolated polypeptide or polypeptide complex according to Embodiments 1-192, or 257-321.

### EXAMPLES

### Example 1: TROP2 Polypeptide Complex Binding

The TROP2-CD3 polypeptide complexes of Table 7 were evaluated for TROP2 and CD3ε binding.

TABLE 7
Polypeptide complexes

| Polypeptide | | | | CD3 | | |
|---|---|---|---|---|---|---|
| complex | Form | Fab Mask | CD3 | Mask | Cleavable linker | sdA |
| PC1 | Vh | Non masked, SEQ ID NO. 13 (normal orientation) | | | | |
| PC5 | Vh | SEQ ID | SEQ ID NO. | SEQ ID | LSGRSDAGSPLGLAG | SEQ ID |
| | | NO. 24 | 13 | NO. 27 | (SEQ ID NO: 57) | NO: 69 |
| PC6 | Vh | SEQ ID | SEQ ID NO. | SEQ ID | LSGRSDAGSPLGLAG | SEQ ID |
| | | NO. 23 | 13 | NO. 27 | (SEQ ID NO: 57) | NO: 69 |
| PC2 | Vl | Non masked, SEQ ID NO. 13 (flipped orientation) | | | | |
| PC7 | Vl | SEQ ID | SEQ ID NO. | SEQ ID | LSGRSDAGSPLGLAG | SEQ ID |
| | | NO. 24 | 13 | NO. 27 | (SEQ ID NO: 57) | NO: 69 |
| PC8 | Vl | SEQ ID | SEQ ID NO. | SEQ ID | LSGRSDAGSPLGLAG | SEQ ID |
| | | NO. 23 | 13 | NO. 27 | (SEQ ID NO: 57) | NO: 69 |
| PC13 | Vh | SEQ ID | SEQ ID NO. | SEQ ID | LSGRSDAGSPLGLAG | SEQ ID |
| | | NO. 24 | 13 | NO. 27 | (SEQ ID NO: 57) | NO: 73 |
| PC14 | Vh | SEQ ID | SEQ ID NO. | SEQ ID | LSGRSDAGSPLGLAG | SEQ ID |
| | | NO. 23 | 13 | NO. 27 | (SEQ ID NO: 57) | NO: 73 |
| PC15 | Vl | SEQ ID | SEQ ID NO. | SEQ ID | LSGRSDAGSPLGLAG | SEQ ID |
| | | NO. 24 | 13 | NO. 27 | (SEQ ID NO: 57) | NO: 73 |
| PC16 | Vl | SEQ ID | SEQ ID NO. | SEQ ID | LSGRSDAGSPLGLAG | SEQ ID |
| | | NO. 23 | 13 | NO. 27 | (SEQ ID NO: 57) | NO: 73 |

The polypeptide complex molecules of Table 7 were evaluated for their ability to bind TROP2 as well as CD3 in a standard enzyme linked immunosorbent assay (ELISA) format. Polypeptide complex binding kinetics of TROP2 or CD3 were measured before and after protease treatment. Briefly, biotinylated antigen was captured on neutravidin coated plates. Polypeptide complex molecules were treated with active matriptase (MTSP1) where indicated. Polypeptide complex molecules diluted in buffer were then added to the antigen coated plates. Bound polypeptide complex was detected using a standard horse radish peroxidase conjugate secondary antibody. The concentration of polypeptide complex required to achieve 50% maximal signal (EC50) was calculated.

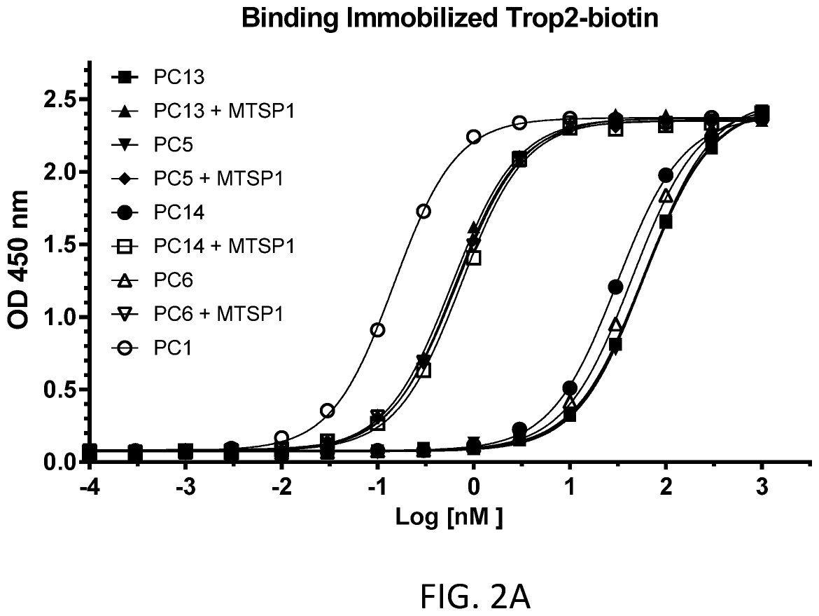

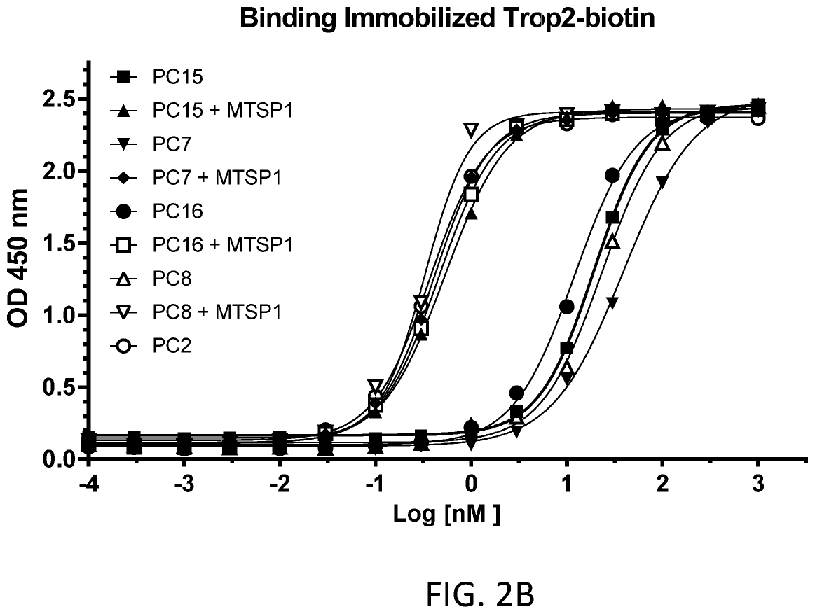

FIGS. 2A and 2B show representative ELISAs of TROP2 binding. This data is summarized in Tables 8 and 9. The masked polypeptide complexes of PC13 and PC5 have EC50s about 400 fold higher than the unmasked polypeptide complex of PC1, protease treatment rescues the EC50s of PC13 and PC5 to only about 4 fold higher than the unmasked polypeptide complex. Similarly, the masked polypeptide complex of PC6 has an EC50 300 fold higher than PC1, and protease treatment rescues this to about 4.5 fold higher than PC1. The masked polypeptide complex of PC14 has an EC50 about 200 fold higher than PC1, and protease treatment rescues this to about 5 fold higher than PC1.

The polypeptide complexes with the flipped orientation show a similar pattern. PC7 and PC8 have EC50s greater than 100 fold and 60 fold higher than PC2 respectively. PC15 and PC16 have EC50s greater than 50 fold and 30 fold higher than PC2 respectively. Protease treatment rescued binding of all four complexes such that the EC50s were close to that of PC2.

TABLE 8
TROP2 binding

| | | | PC13 + | | PC5 + | | PC14 + | | PC6 + |
|---|---|---|---|---|---|---|---|---|---|
| TROP2 | PC1 | PC13 | MTSP1 | PC5 | MTSP1 | PC14 | MTSP1 | PC6 | MTSP1 |
| EC50 nM | 0.1477 | 58.42 | 0.5907 | 60.25 | 0.6337 | 30.88 | 0.7302 | 44.37 | 0.6686 |
| Fold | 1× | 395.5× | 4× | 407.9× | 4.3× | 209.1× | 4.9× | 300.4× | 4.5× |
| shift | | | | | | | | | |

TABLE 9
TROP2 binding

| | | | PC15 + | | PC7 + | | PC16 + | | PC 8 + |
|---|---|---|---|---|---|---|---|---|---|
| TROP2 | PC2 | PC15 | MTSP1 | PC7 | MTSP1 | PC16 | MTSP1 | PC8 | MTSP1 |
| EC50 nM | 0.3697 | 19.39 | 0.5306 | 39.38 | 0.4087 | 11.98 | 0.4572 | 23.25 | 0.3289 |
| Fold | 1× | 52.4× | 1.4× | 106.5× | 1.1× | 32.4× | 1.2× | 62.9× | 0.9× |
| shift | | | | | | | | | |

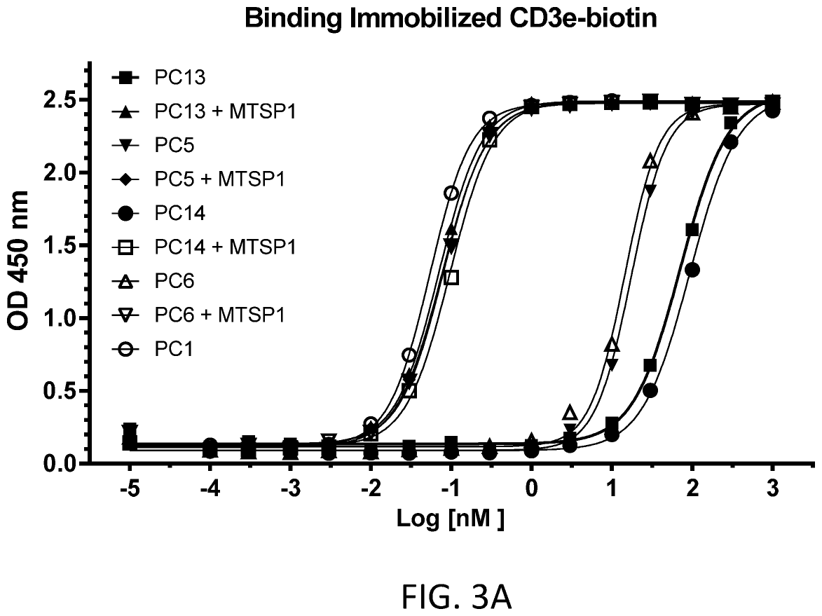

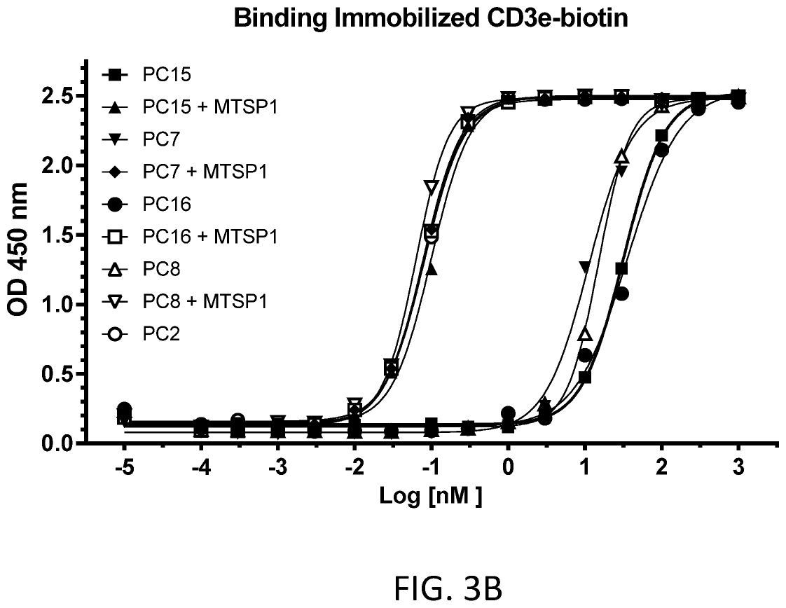

FIGS. 3A and 3B show representative ELISAs of CD3 binding. This data is summarized in Tables 10 and 11. The masked polypeptide complexes of PC13 and PC14 respectively had an EC50s about 1313 and 1723 fold higher than the unmasked polypeptide complex of PC1, protease treatment rescued the EC50s to under 2 fold higher than the unmasked polypeptide complex. Similarly, the masked polypeptide complexes of PC5 and PC6 respectively had EC50s about 321 and 267 fold higher than PC1, and protease treatment rescued the EC50s of both to about 1.5 fold higher than PC1. The polypeptide complexes with the flipped orientation show a similar pattern. PC15 and PC16 had EC50s 390 fold and 442 fold higher than PC2 respectively. PC7 and PC8 have EC50s 138 fold and 165 fold higher than PC2 respectively. Protease treatment rescued the EC50s of all four complexes such that the EC50s are close to that of PC2.

TABLE 10
CD3 binding

| | | | PC13 + | | PC5 + | | PC14 + | | PC6 + |
|---|---|---|---|---|---|---|---|---|---|
| CD3e | PC1 | PC13 | MTSP1 | PC5 | MTSP1 | PC14 | MTSP1 | PC6 | MTSP1 |
| EC50 nM | 0.05402 | 70.96 | 0.07024 | 17.34 | 0.07634 | 93.10 | 0.09582 | 14.46 | 0.07916 |
| Fold | 1× | 1313.6× | 1.3× | 321× | 1.4× | 1723.4× | 1.8× | 267.7× | 1.5× |
| shift | | | | | | | | | |

TABLE 11
CD3 binding

| | | | PC15 + | | PC7 + | | PC16 + | | PC8 + |
|---|---|---|---|---|---|---|---|---|---|
| CD3e | PC2 | PC15 | MTSP1 | PC7 | MTSP1 | PC16 | MTSP1 | PC8 | MTSP1 |
| EC50 nM | 0.08100 | 31.62 | 0.09884 | 11.21 | 0.07748 | 35.79 | 0.07767 | 15.02 | 0.06267 |
| Fold | 1× | 390.4× | 1.2× | 138.4× | 1× | 441.9× | 1× | 185.4× | 0.8× |
| shift | | | | | | | | | |

### Example 2: Polypeptide Complex Mediated Tumor Cytotoxicity and T Cell Activation

Polypeptide complexes were evaluated in a functional in vitro tumor cell killing assay using the TROP2 positive tumor cell lines HCT116 and MDAMB231. Tumor cell killing was measured using a real time cell analyzer from Acea Biosciences that relies on sensor impedance measurements (cell index) that increased as tumor cells adhere, spread, and expand on the surface of the sensor. Likewise, as the tumor cells were killed the impedance decreased. 25,000 tumor cells were added per well and allowed to adhere overnight. The following day polypeptide complexes titrated in human serum supplemented medium along with 75,000 CD8+ T cells were added to the wells. Cell index measurements were taken every 10 minutes for an additional 96 hours. The cell index times number of hours (tumor cell growth kinetics) was then plotted versus concentration of polypeptide complex where the concentration required to reduce the tumor growth 50% (IC50) was calculated using Graphpad Prism.

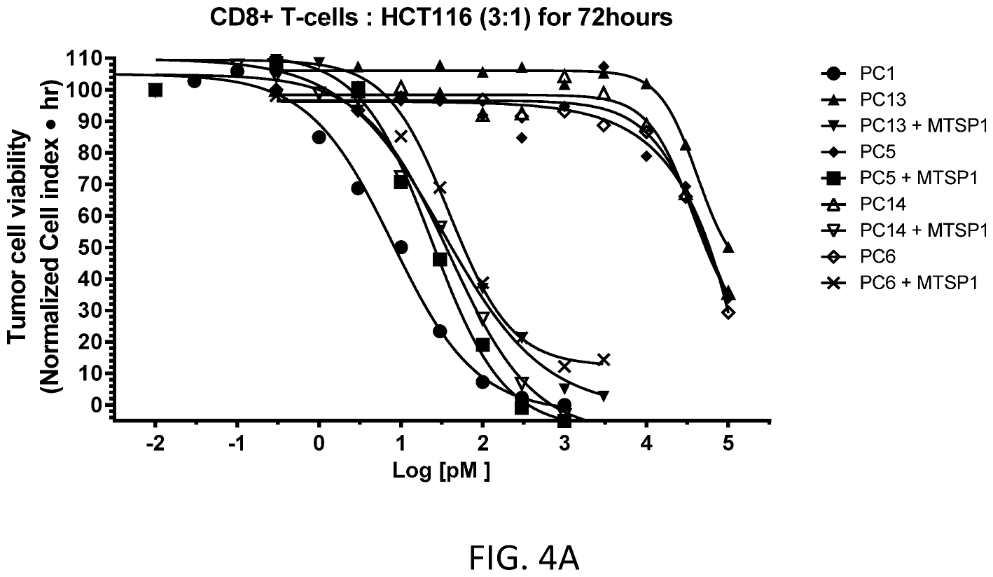

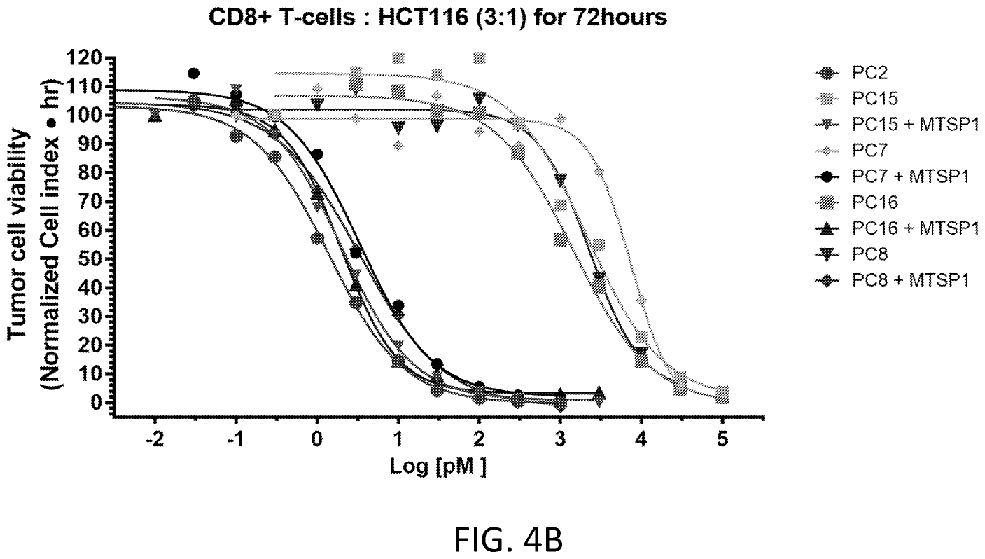

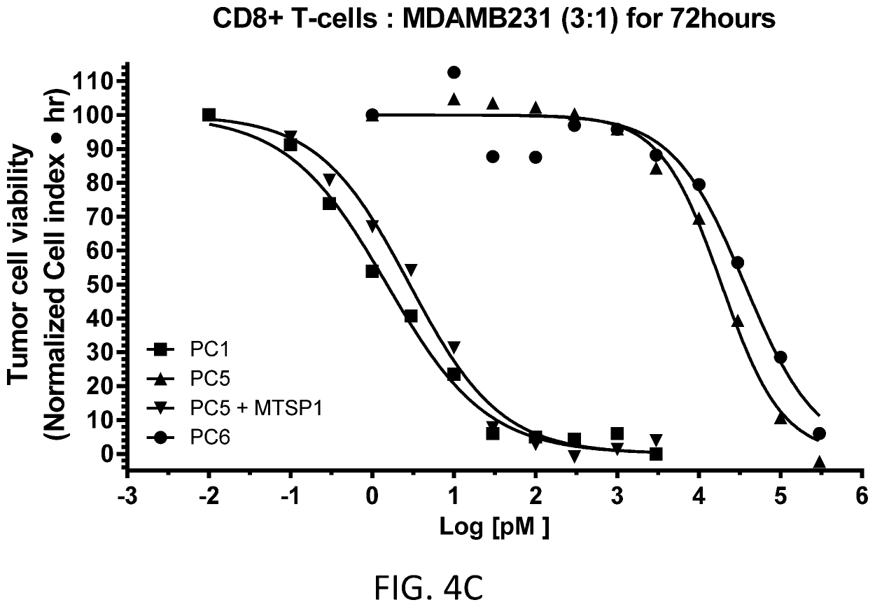

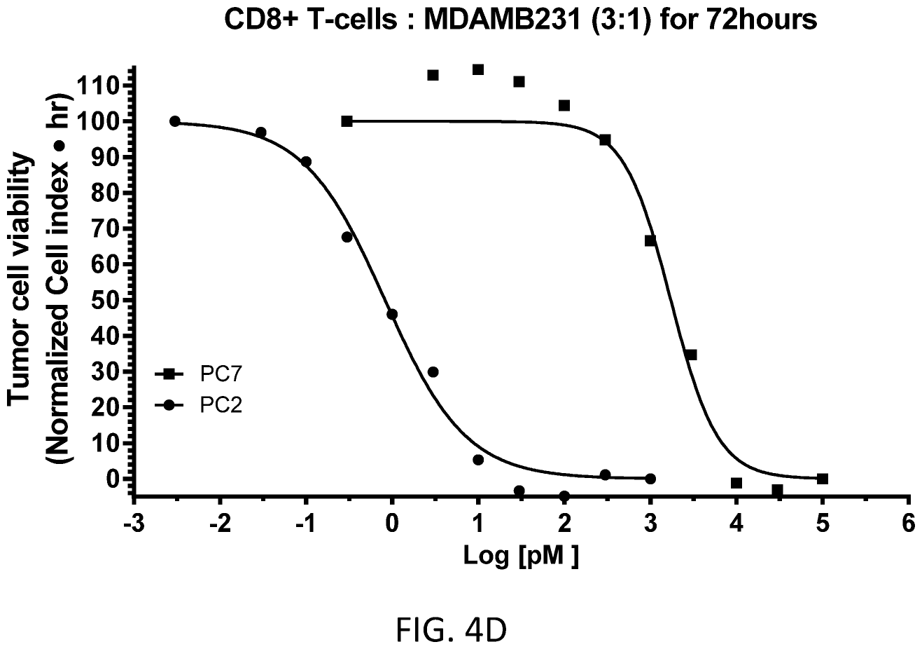

The HCT116 tumor cell line has a TROP2 density of <10,000 copies per cell. FIGS. 4A and 4B show representative viability data for HCT116. This data is summarized in Tables 12 and 13. The masked polypeptide complex of PC5 has an IC50 greater than 6000 fold higher than the unmasked polypeptide complex of PC1, protease treatment rescues the IC50 to only about 3 fold higher than the unmasked polypeptide complex. Similarly, the masked polypeptide complex of PC6 has an IC50 about 5750 fold higher than PC1, and protease treatment rescues this to about 8.6 fold higher than PC1. The polypeptide complexes with the flipped orientation show a similar pattern. PC7 and PC8 have IC50s 4567 fold and 1707 fold higher than PC2 respectively. Protease treatment rescues both of these complexes such that the IC50s are less than 3 fold that of PC2.

TABLE 12
HCT116 cell assay

| HCT116 | | | PC13 + | | PC5 + | | PC14 + | | PC6 + |
|---|---|---|---|---|---|---|---|---|---|
| 72 hr | PC1 | PC13 | MTSP1 | PC5 | MTSP1 | PC14 | MTSP1 | PC6 | MTSP1 |
| IC50 pM | 8.604 | 98.305 | 45.06 | 56.038 | 25.69 | 58.112 | 34.62 | 49.533 | 74.25 |
| Fold | 1× | 11,425.5× | 5.2× | 6.513× | 3× | 6.754.1× | 4× | 5.757× | 8.6× |
| shift | | | | | | | | | |

TABLE 13
HCT116 cell assay

| HCT116 | | | PC15 + | | PC7 + | | PC16 + | | PC8 + |
|---|---|---|---|---|---|---|---|---|---|
| 72 hr | PC2 | PC15 | MTSP1 | PC7 | MTSP1 | PC16 | MTSP1 | PC8 | MTSP1 |
| IC50 pM | 1.523 | 3.253 | 2.46 | 6.955 | 4.488 | 1.715 | 2.34 | 2.601 | 3.676 |
| Fold | 1× | 2,135.9× | 1.6× | 4,566.6× | 2.9× | 1,126.1× | 1.5× | 1,707.8× | 2.4× |
| shift | | | | | | | | | |

The MDAMB231 tumor cell line has a TROP2 density of about 32,000 copies per cell. FIGS. 4C and 4D show representative viability data for MDAMB231. This data is summarized in Table 14. The masked polypeptide complex of PC5 has an IC50 greater than 12000 fold higher than the unmasked polypeptide complex of PC1, protease treatment rescues the IC50 to less than 2 fold higher than the unmasked polypeptide complex. The masked polypeptide complex of PC6 has an IC50 about 24,000 fold higher than PC1. The polypeptide complexes with the flipped orientation show a similar pattern. PC7 has an IC50 6864 fold higher than PC2.

TABLE 14
MDAMB231 cell assay

| MDAMB231 | | | PC5 + | | | |
|---|---|---|---|---|---|---|
| 72 hr | PC1 | PC5 | MTSP1 | PC6 | PC7 | PC2 |
| IC50 pM | 1.514 | 18.951 | 2.868 | 36.549 | 1.817 | 0.2647 |
| Fold shift | 1× | 12.517× | 1.9× | 24.141× | 6.864× | 1× |

Some further polypeptide complexes with different Cd3 masking sequences are shown in Table 14. The TROP2-CD3 polypeptide complexes of Table 15 were evaluated for TROP2 and CD3ε binding as described above.

TABLE 15
Polypeptide complexes

| Polypeptide | | | | | |
|---|---|---|---|---|---|
| complex | Fab Mask | CD3 | CD3 Mask | Cleavable linker | sdA |
| PC1 | Non masked TCE, SEQ ID NO. 13 (normal orientation) | | | | |
| PC9 | SEQ ID NO. | SEQ ID NO. | SEQ ID NO. | LSGRSDAGSPLGLAG | SEQ ID |
| | 24 | 13 | 26 | (SEQ ID NO: 57) | NO: 69 |
| PC11 | SEQ ID NO. | SEQ ID NO. | SEQ ID NO. | LSGRSDAGSPLGLAG | SEQ ID |
| | 23 | 13 | 26 | (SEQ ID NO: 57) | NO: 69 |
| PC2 | Non masked TCE, SEQ ID NO. 13 (flipped orientation) | | | | |
| PC10 | SEQ ID NO. | SEQ ID NO. | SEQ ID NO. | LSGRSDAGSPLGLAG | SEQ ID |
| | 24 | 13 | 26 | (SEQ ID NO: 57) | NO: 69 |
| PC12 | SEQ ID | SEQ ID | SEQ ID | LSGRSDAGSPLGLAG | SEQ ID |
| | NO. 23 | NO. 13 | NO. 26 | (SEQ ID NO: 57) | NO: 69 |

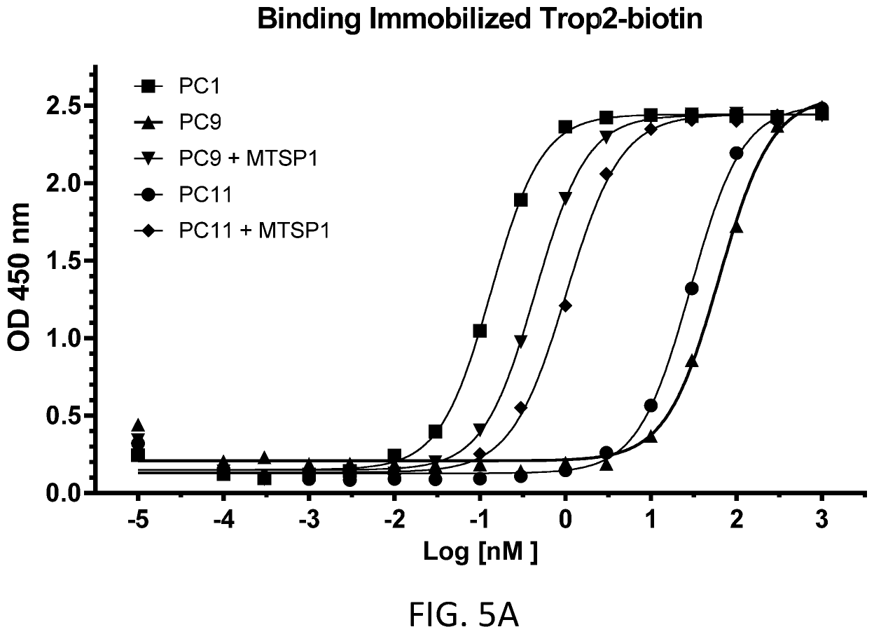

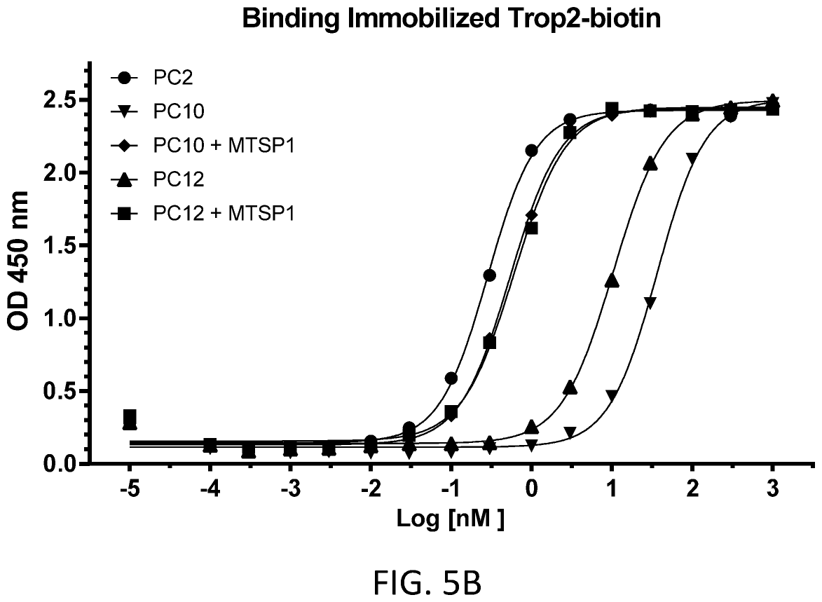

FIGS. 5A and 5B show representative ELISAs of TROP2 binding. This data is summarized in Tables 16 and 17. The masked polypeptide complex of PC9 has an EC50 greater than 450 fold higher than the unmasked polypeptide complex of PC1, protease treatment rescues the EC50 to only about 3.3 fold higher than the unmasked polypeptide complex. Similarly, the masked polypeptide complex of PC11 has an EC50 greater than 200 fold higher than PC1, and protease treatment rescues this to about 8 fold higher than PC1. The polypeptide complexes with the flipped orientation show a similar pattern. PC10 and PC12 have EC50s about 128 fold and 37 fold higher than PC2 respectively. Protease treatment rescues both of these complexes such that the EC50s are about twice that of PC2.

TABLE 16
TROP2 binding

| | | | PC9 + | | PC11 + |
|---|---|---|---|---|---|
| TROP2 | PC1 | PC9 | MTSP1 | PC11 | MTSP1 |
| EC50 | 0.1344 | 62.57 | 0.4432 | 28.90 | 1.023 |
| nM | | | | | |
| Fold | 1x | 465.6x | 3.3x | 215x | 7.6x |
| shift | | | | | |

TABLE 17
TROP2 binding

| | | | PC10 + | | PC12 + |
|---|---|---|---|---|---|
| TROP2 | PC2 | PC10 | MTSP1 | PC12 | MTSP1 |
| EC50 | 0.2833 | 36.46 | 0.5404 | 10.37 | 0.6020 |
| nM | | | | | |
| Fold | 1x | 128.7x | 1.9x | 36.6x | 2.1x |
| shift | | | | | |

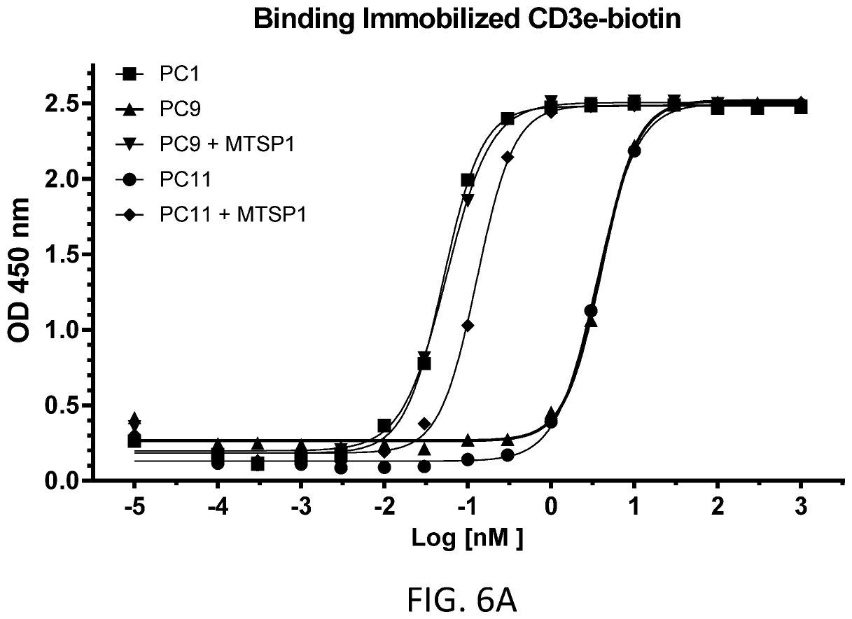

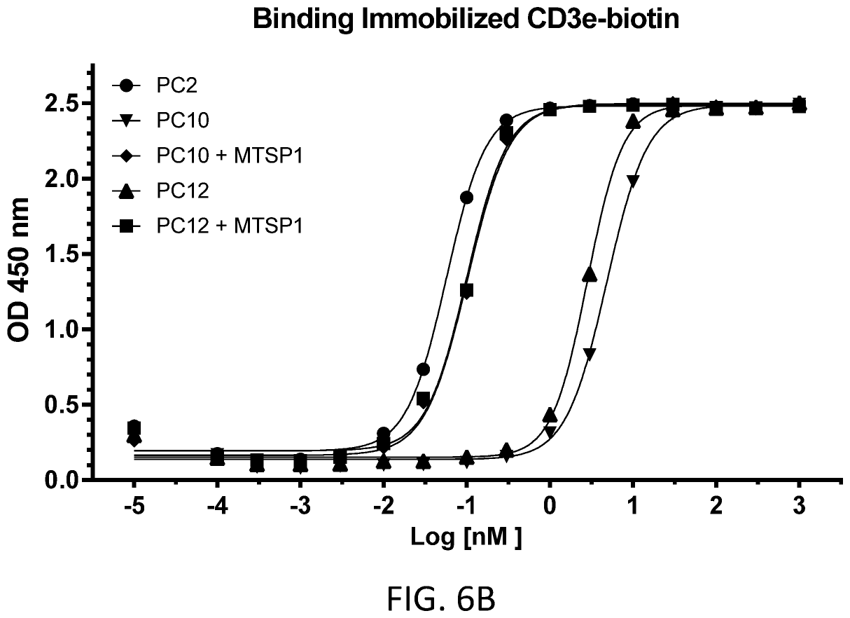

FIGS. 6A and 6B show representative ELISAs of CD3e binding. This data is summarized in Tables 18 and 19. The masked polypeptide complex of PC9 has an EC50 greater than 78 fold higher than the unmasked polypeptide complex of PC1, protease treatment rescues the EC50 to only about the same as the unmasked polypeptide complex. Similarly, the masked polypeptide complex of PC11 has an EC50 about 70 fold higher than PC1, and protease treatment rescues this to about 2.5 fold higher than PC1. The polypeptide complexes with the flipped orientation show a similar pattern. PC10 and PC12 have EC50s about 85 fold and 50 fold higher than PC2 respectively. Protease treatment rescues both of these complexes such that the EC50s are about twice that of PC2.

TABLE 18
CD3 binding

| | | | PC9 + | | PC11 + |
|---|---|---|---|---|---|
| CD3e | PC1 | PC9 | MTSP1 | PC11 | MTSP1 |
| EC50 nM | 0.05085 | 3.976 | 0.05538 | 3.526 | 0.1286 |
| Fold shift | 1x | 78.2x | 1.1x | 69.3x | 2.5x |

TABLE 19
CD3 binding

| | | | PC10 + | | PC12 + |
|---|---|---|---|---|---|
| CD3e | PC2 | PC10 | MTSP1 | PC12 | MTSP1 |
| EC50 nM | 0.05690 | 4.829 | 0.1019 | 2.819 | 0.09992 |
| Fold shift | 1x | 84.9x | 1.8x | 49.5x | 1.8x |

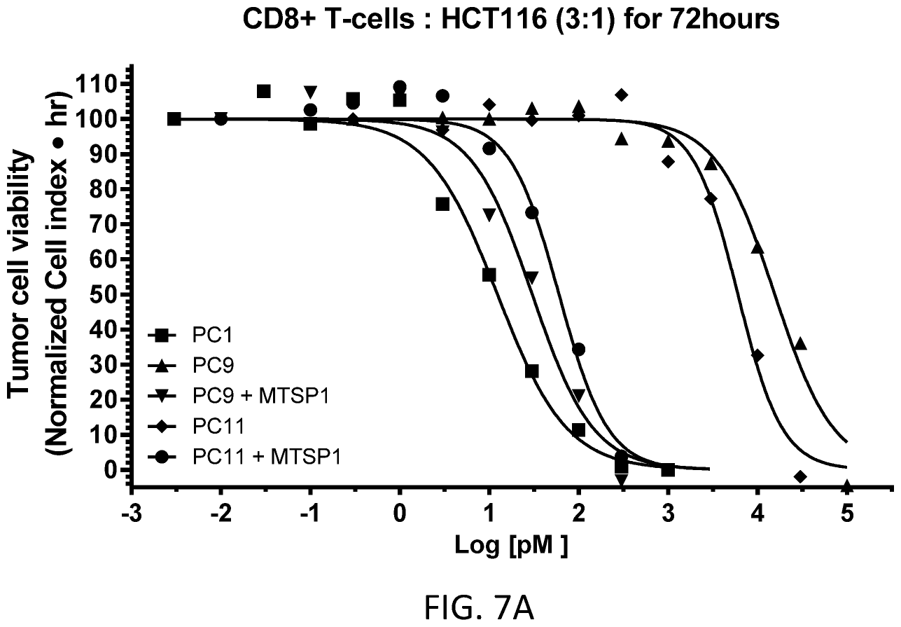

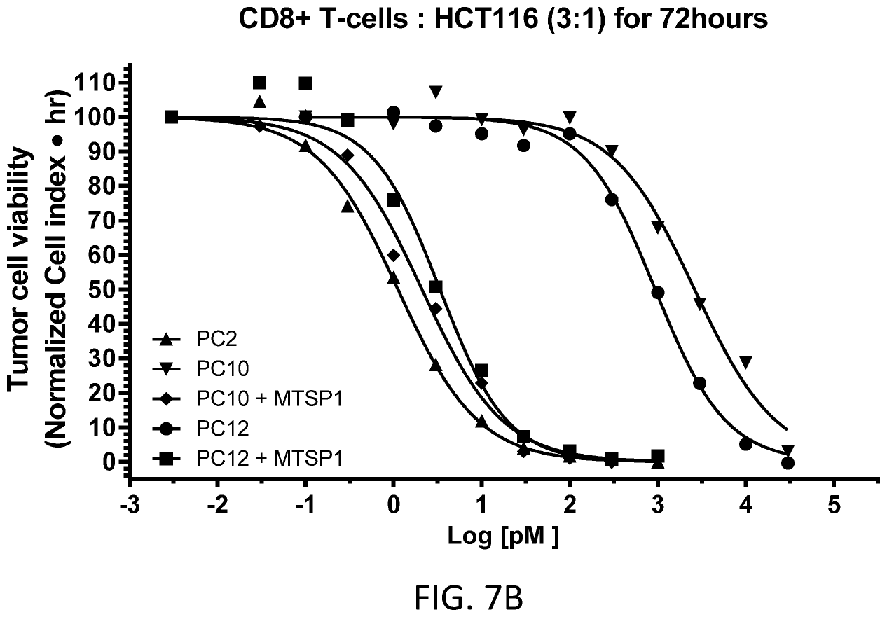

The polypeptide complexes of Table 14 were also evaluated in a functional in vitro tumor cell killing assay using HCT116 as described above. The HCT116 tumor cell line has a TROP2 density of <10,000 copies per cell. FIGS. 7A and 7B show representative viability data for HCT116. This data is summarized in Table 20. The masked polypeptide complexes of PC9 and PC11 had IC50s respectively about 1259 and 488 fold higher than the unmasked polypeptide complex of PC1, protease treatment rescues the IC50s to about 2.5 and 5 fold higher than the unmasked polypeptide complex. The masked polypeptide complexes of PC10 and PC12 had IC50s respectively about 2337 and 935 fold higher than the unmasked polypeptide complex of PC2, protease treatment rescues the IC50s to about 2 and 3 fold higher than the unmasked polypeptide complex.

TABLE 20
HCT116 cell assay

| HCT116 | | | PC9 + | | PC11 + | | | PC10 + | | PC12 + |
|---|---|---|---|---|---|---|---|---|---|---|
| 72 hr | PC1 | PC9 | MTSP1 | PC11 | MTSP1 | PC2 | PC10 | MTSP1 | PC12 | MTSP1 |
| IC50 pM | 12.34 | 15.533 | 30.21 | 6.022 | 60.18 | 1.119 | 2.615 | 2.101 | 935.2 | 3.36 |
| Fold shift | 1× | 1.259× | 2.45× | 488.0× | 4.88× | 1 | 2.337× | 1.88× | 835.7× | 3.0× |

### Example 3: Polypeptide Complex Mediated Tumor Cell Killing

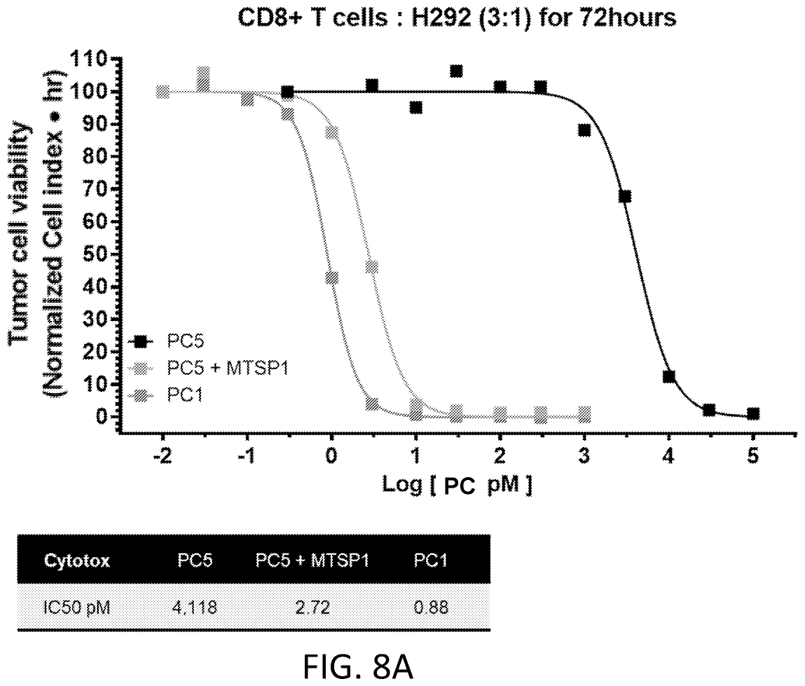

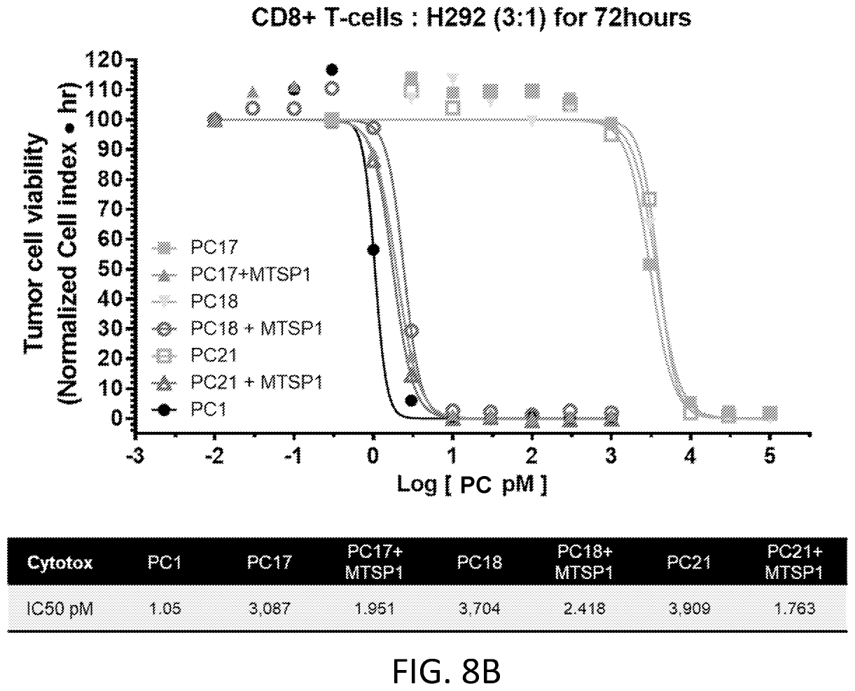

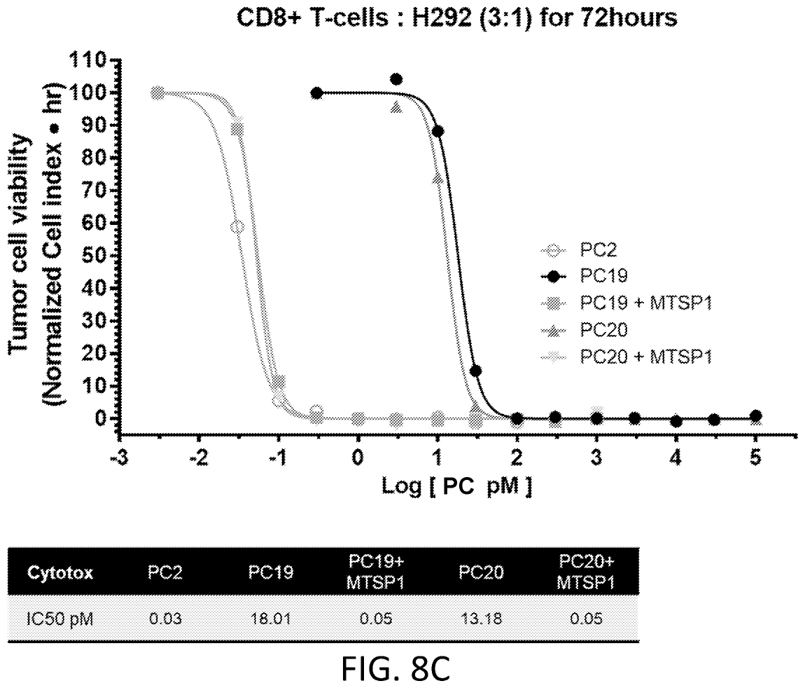

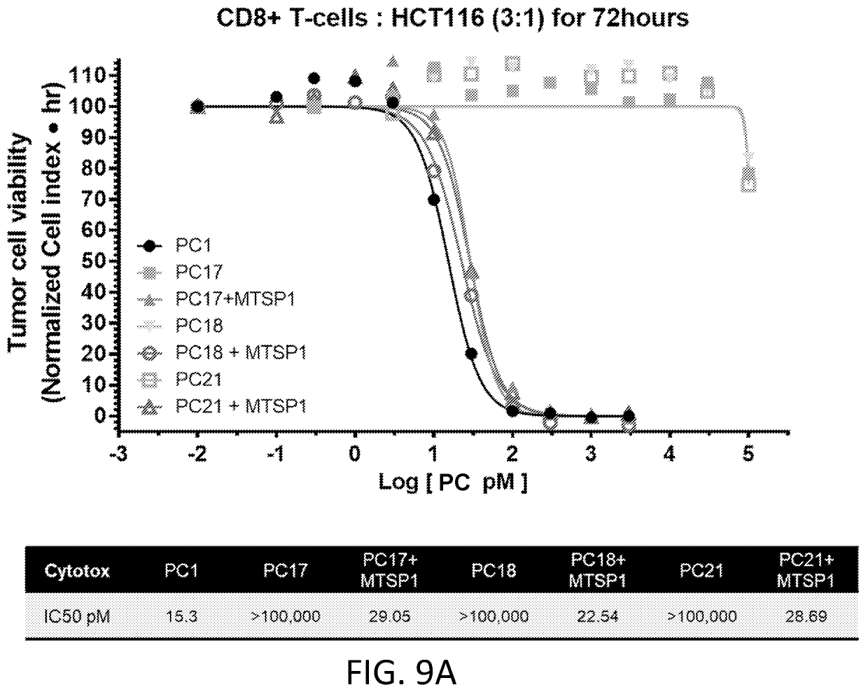

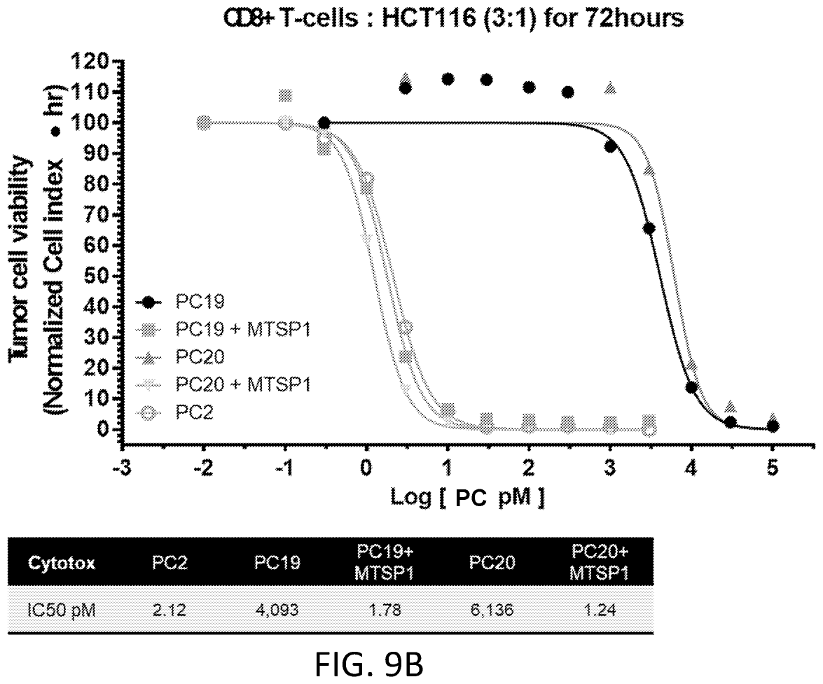

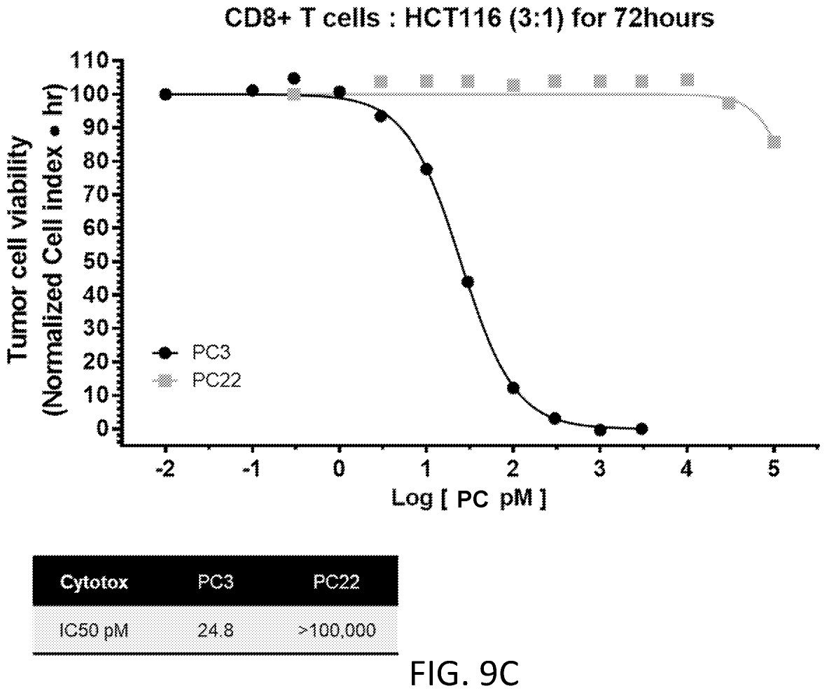

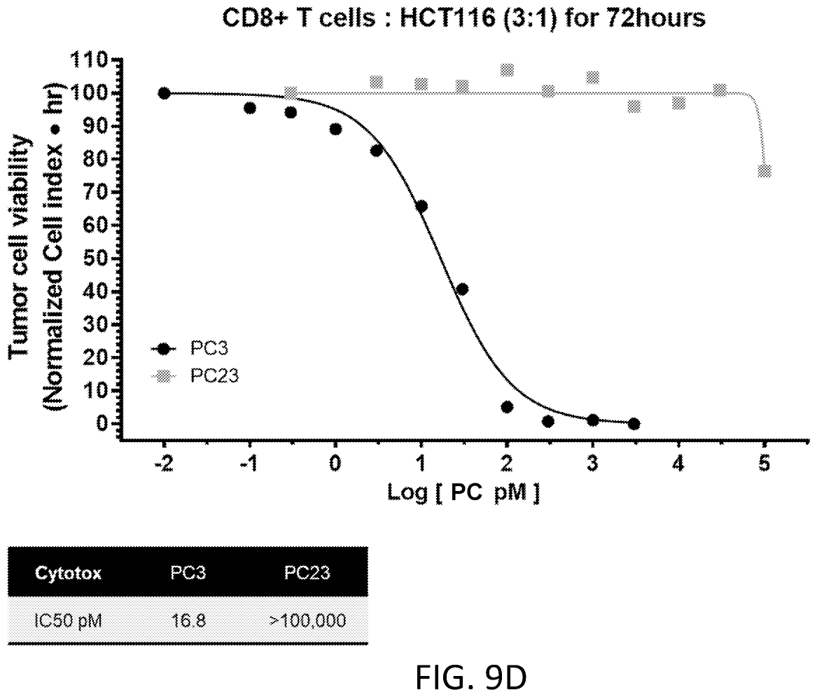

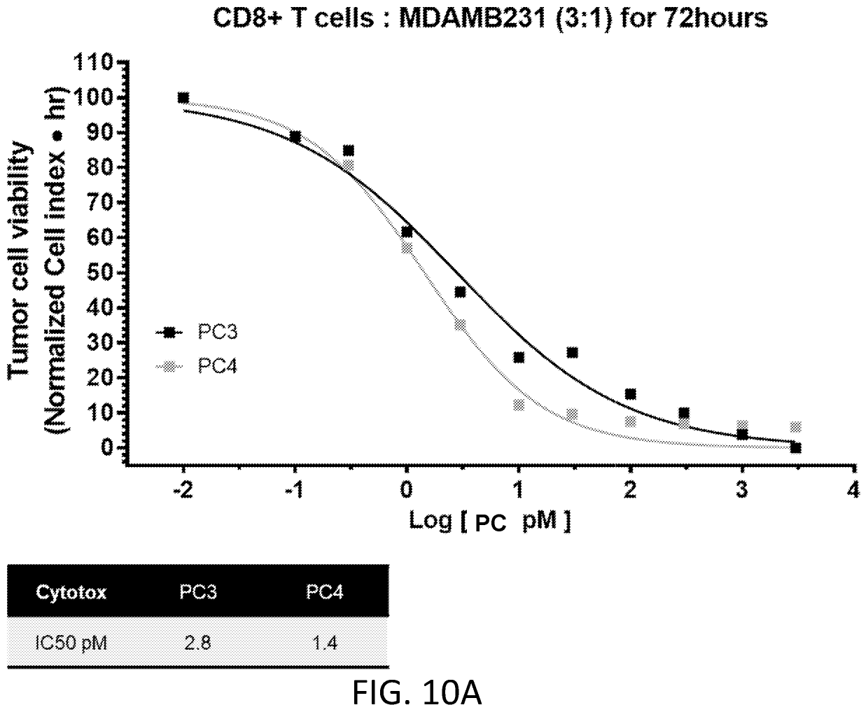

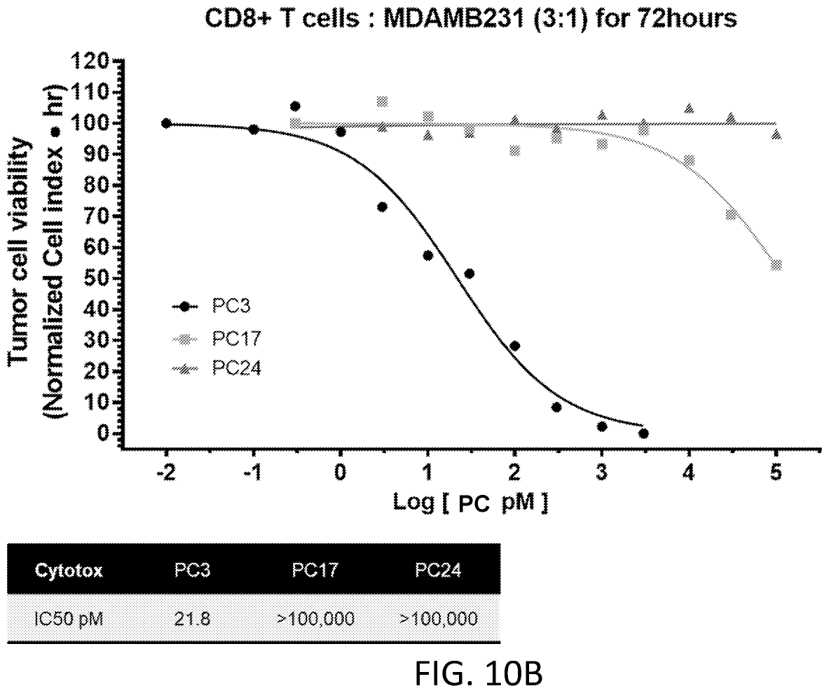

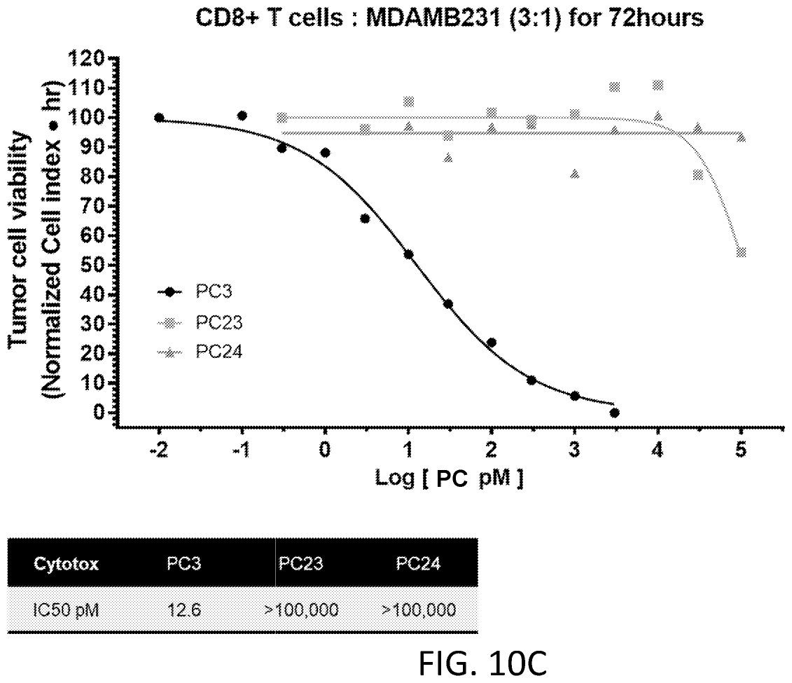

Polypeptide complexes were evaluated in a functional in vitro tumor cell killing assay using the TROP2 positive tumor cell lines HCT116, NCI-H292, and MDAMB231. Tumor cell killing was measured using an xCelligence real time cell analyzer from Agilent that relies on sensor impedance measurements (cell index) that increased as tumor cells adhere, spread, and expand on the surface of the sensor. Likewise, as the tumor cells were killed the impedance decreased. 10,000 tumor cells were added per well and allowed to adhere overnight on a 96 well E-Plate. The following day polypeptide complexes titrated in human serum supplemented medium along with 30,000 CD8+ T cells were added to the wells. Cell index measurements were taken every 10 minutes for an additional 72 hours. The cell index times number of hours (tumor cell growth kinetics) was then plotted versus concentration of polypeptide complex where the concentration required to reduce the tumor growth 50% (IC50) was calculated using Graphpad Prism software. Data for NCI-H292 (H292) is seen in FIGS. 8A-8C. Data for HCT116 is seen in FIGS. 9A-9D. Data for MDMAB231 is seen in FIGS. 10A-10C.

### Example 4: Polypeptide Complex Pharmacokinetics in Cynomolgus Monkey

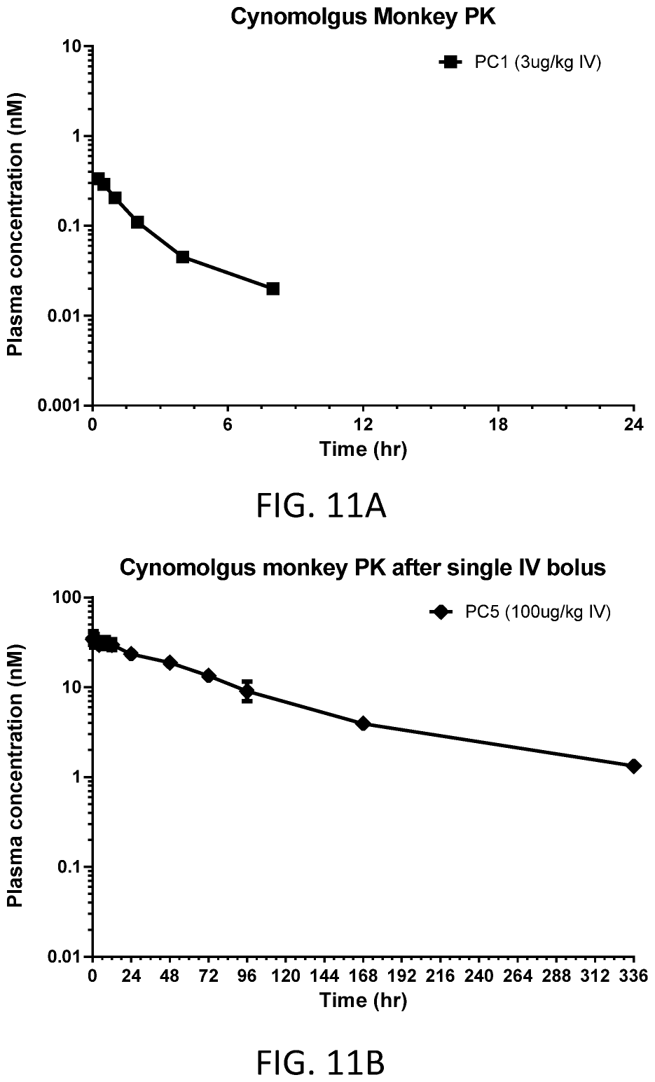

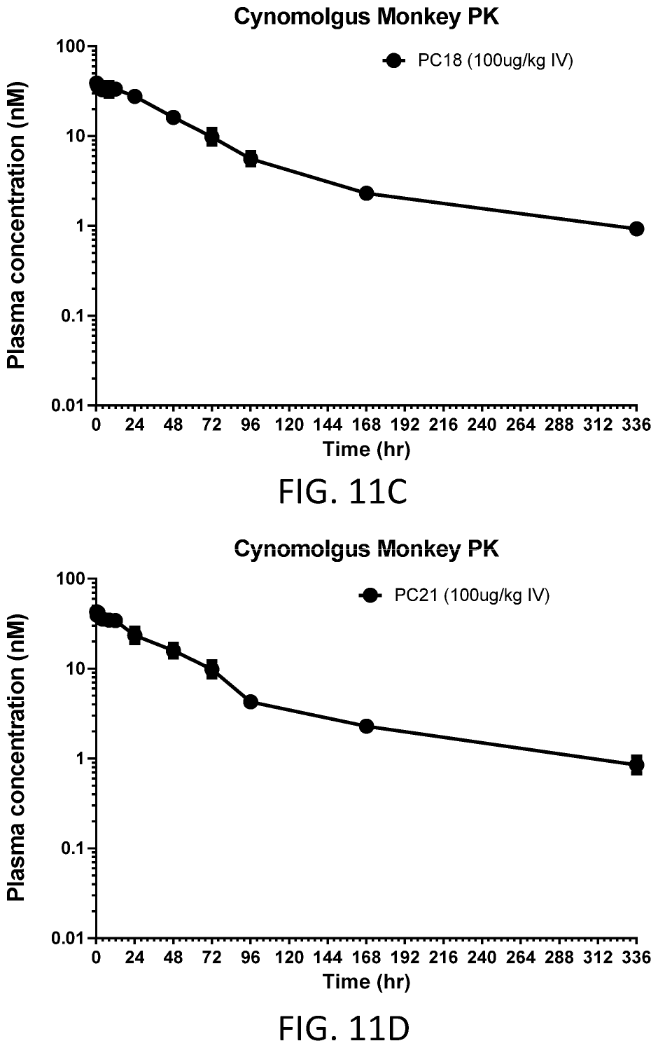

Pharmacokinetics and exploratory safety of polypeptide molecules were evaluated in cynomolgus monkeys. Briefly, cynomolgus monkeys of approximately 3 kg bodyweight were administered polypeptides as an IV bolus and observed daily for signs of adverse events. No in-life adverse events were observed. After dosing, blood was collected in K2 EDTA tubes at specific timepoints and processed to plasma. Plasma was stored frozen until analysis. Concentration of polypeptide molecules in plasma was measured via standard ELISA techniques relative to a reference standard diluted in control cyno plasma. Plasma concentration curves were fit to a standard two phase exponential equation representing distribution and elimination phases. Fitting of pharmacokinetics enabled the calculation of Cmax, half-life, volume of distribution, clearance, and 7 day area under the curve (AUC) shown in Table 21 for TROP2 TCE polypeptide complexes and Tables 22-24 for TROP2 TRACTr polypeptide complexes and FIGS. 11A-11D.

TABLE 21

| | PC1 3 ug/kg | Units |
|---|---|---|
| C_{MAX} | 0.40 | nM |
| t_{1/2} | 1.02 | hr |
| Vd | 0.30 | L |
| VSS | 0.30 | L |
| CL | 67.31 | mL/hr/kg |
| BW | 3.00 | kg |
| 7 day | 49 | nM · min |
| AUC | | |

TABLE 22

| | PC5 100 ug/kg | Units |
|---|---|---|
| C_{MAX} | 34.32 | nM |
| t_{1/2} | 90.16 | hr |
| Vd | 0.09 | L |
| VSS | 0.19 | L |
| CL | 0.23 | mL/hr/kg |
| BW | 3.00 | kg |
| 7 day | 142,182 | nM · min |
| AUC | | |

TABLE 23

| | PC18 100 ug/kg | Units |
|---|---|---|
| C_{MAX} | 37.94 | nM |
| t_{1/2} | 97.31 | hr |
| Vd | 0.08 | L |
| VSS | 0.33 | L |
| CL | 0.19 | mL/hr/kg |
| BW | 3.00 | kg |
| 7 day | 127,094 | nM · min |
| AUC | | |

TABLE 24

| | PC21 100 ug/kg | Units |
|---|---|---|
| C_{MAX} | 42.12 | nM |
| t_{1/2} | 100.73 | hr |
| Vd | 0.07 | L |
| VSS | 0.39 | L |
| CL | 0.17 | mL/hr/kg |
| BW | 3.00 | kg |
| 7 day | 137,581 | nM · min |
| AUC | | |

### Example 5: Polypeptide Complexes in Cynomolgus Cytokine Release

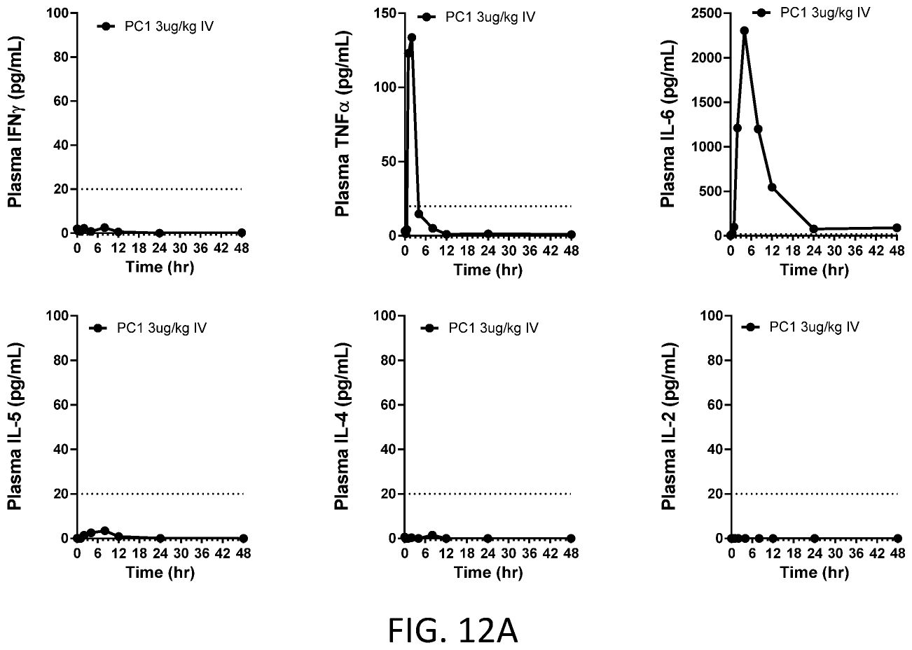

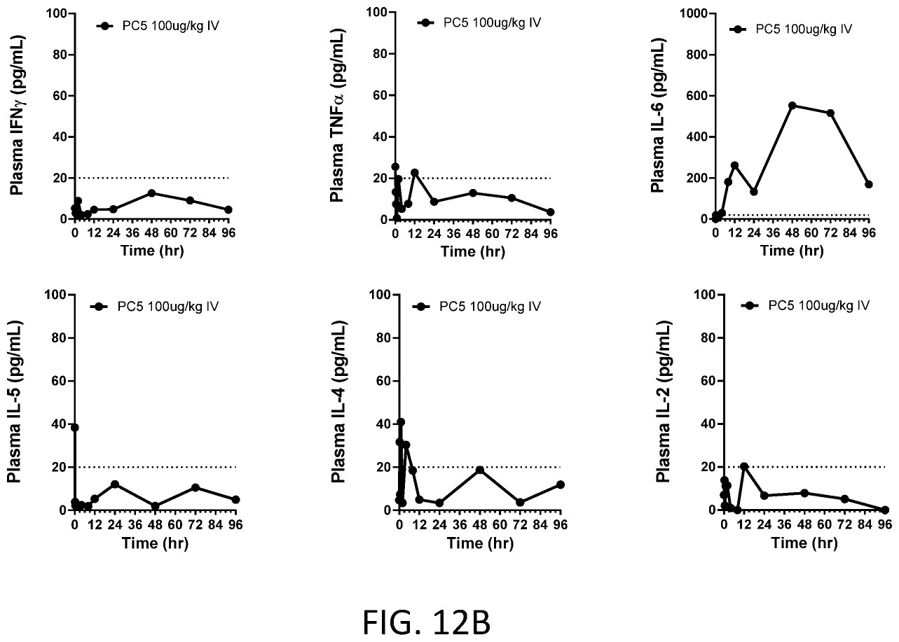

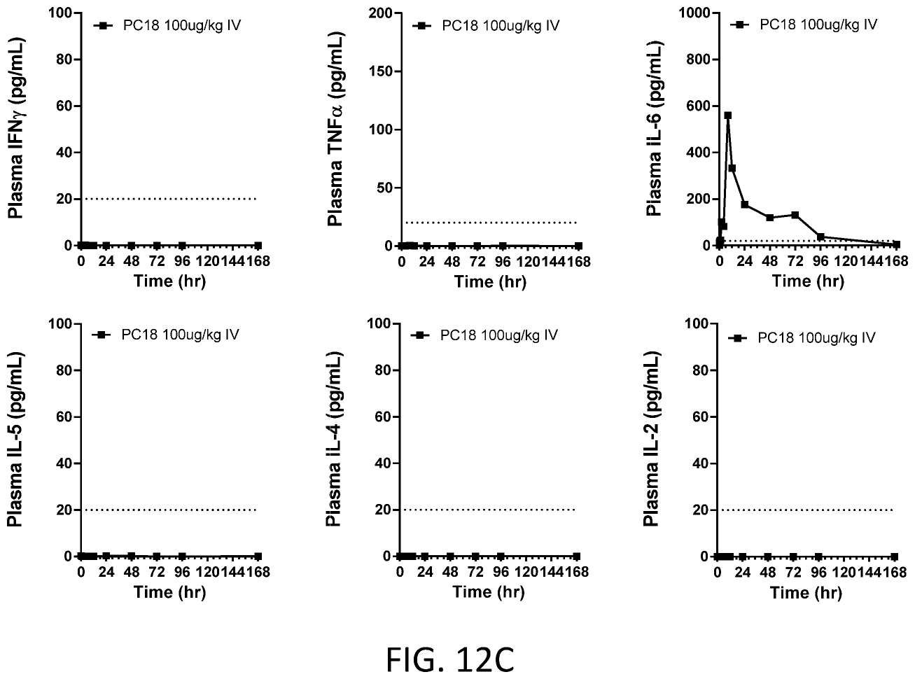

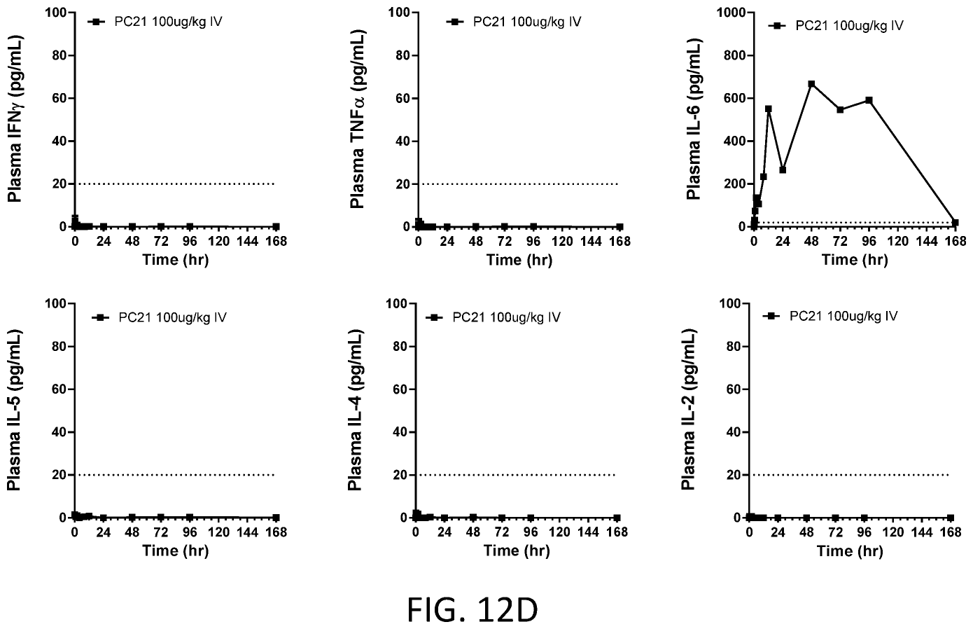

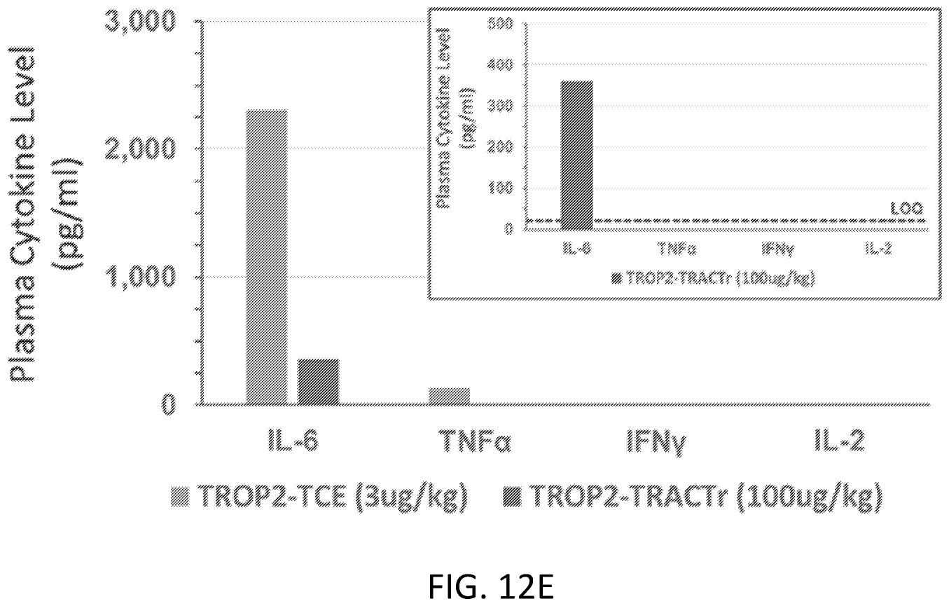

Cytokine release after polypeptide molecule administration by IV bolus was evaluated in cynomolgus monkeys. Briefly, cynomolgus monkeys of approximately 3 kg bodyweight were administered polypeptides as an IV bolus and observed daily for signs of adverse events. No in-life adverse events were observed. After dosing, blood was collected in K2 EDTA tubes at specific timepoints and processed to plasma. Plasma was stored frozen until analysis. Plasma samples were analyzed for cytokines using a non-human primate cytometric Th1/Th2 bead array kit from BD biosciences following the manufacturer's instructions. Interferon gamma, tumor necrosis factor alpha, interleukin 6, interleukin 5, interleukin 4, and interleukin 2 levels in plasma were calculated relative to reference standards provided with the bead array kit. Data is seen in FIGS. 12A-12D.

### Example 6: Polypeptide Complexes in Cynomolgus Toxicity

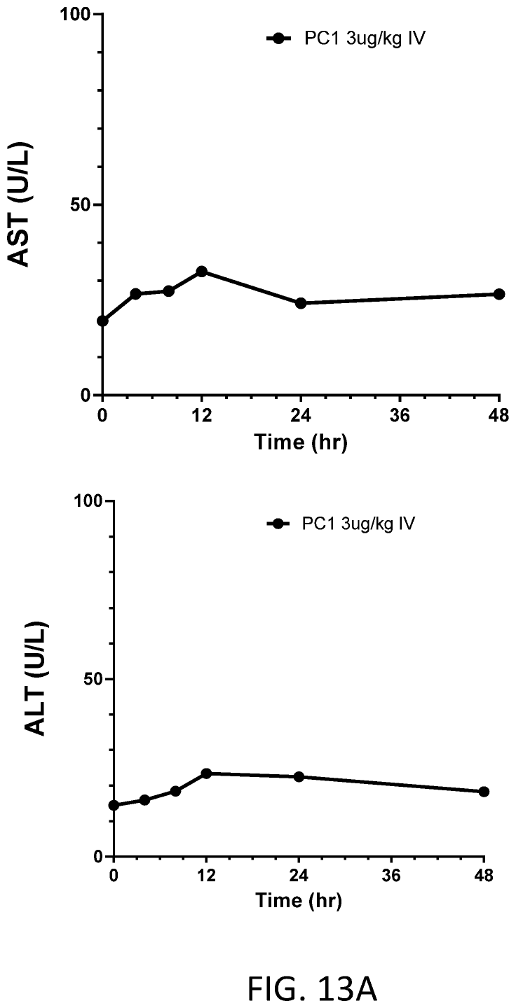

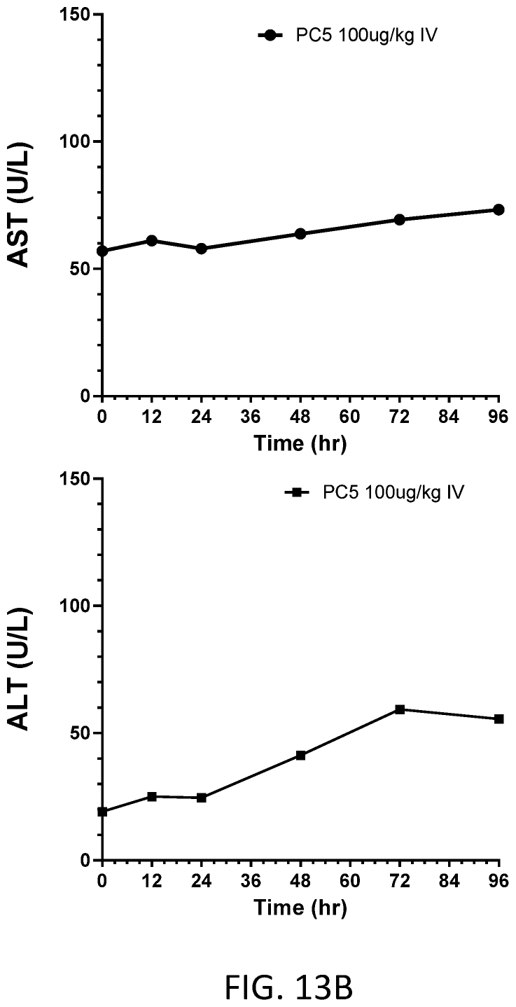

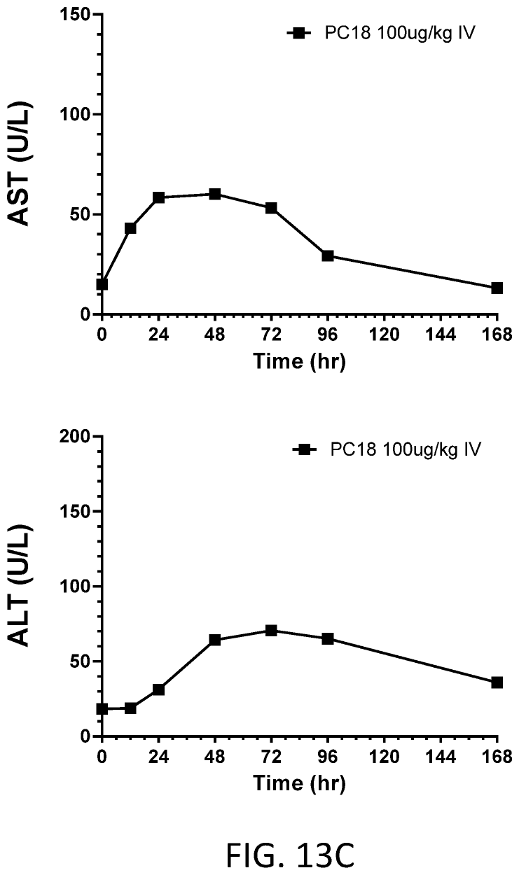

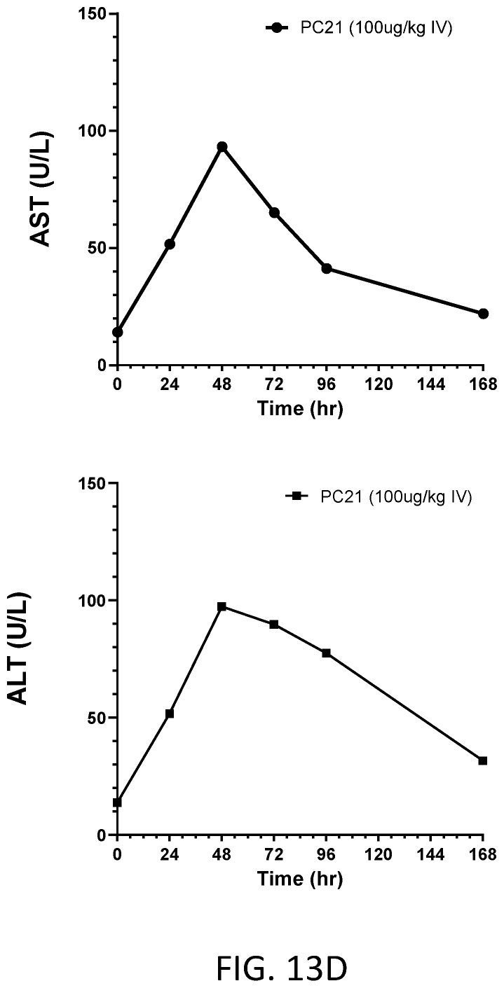

Systemic liver enzymes after polypeptide molecule administration by IV bolus was evaluated in cynomolgus monkeys. Briefly, cynomolgus monkeys of approximately 3 kg bodyweight were administered polypeptides as an IV bolus and observed daily for signs of adverse events. No in-life adverse events were observed. After dosing, blood was collected in K2 EDTA tubes at specific timepoints and processed to plasma. Plasma was stored frozen until analysis. Plasma samples were analyzed for the presence of liver enzymes aspartate transaminase (AST) and alanine aminotransferase (ALT) as signs of potential liver toxicity. AST and ALT levels were remained within the normal ranges for all timepoints tested after dosing suggesting a lack of liver toxicity. AST and ALT were quantified following the instructions provided in a commercially available kit from Millipore. AST and ALT levels were calculated according to manufacturer's instructions relative to a positive control reference standard. Data is seen in FIGS. 13A-13D.

### Example 7: Optimized Phage Library Construction—TROP2 Fab Peptides

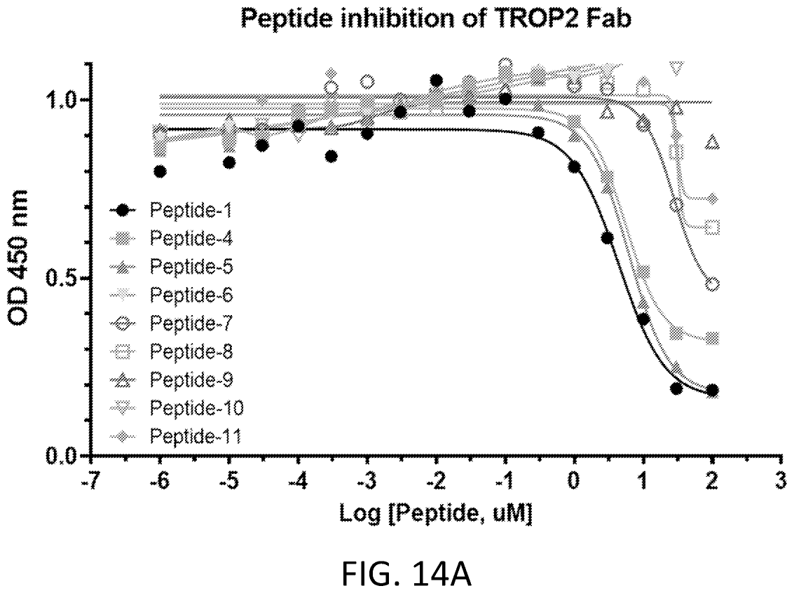

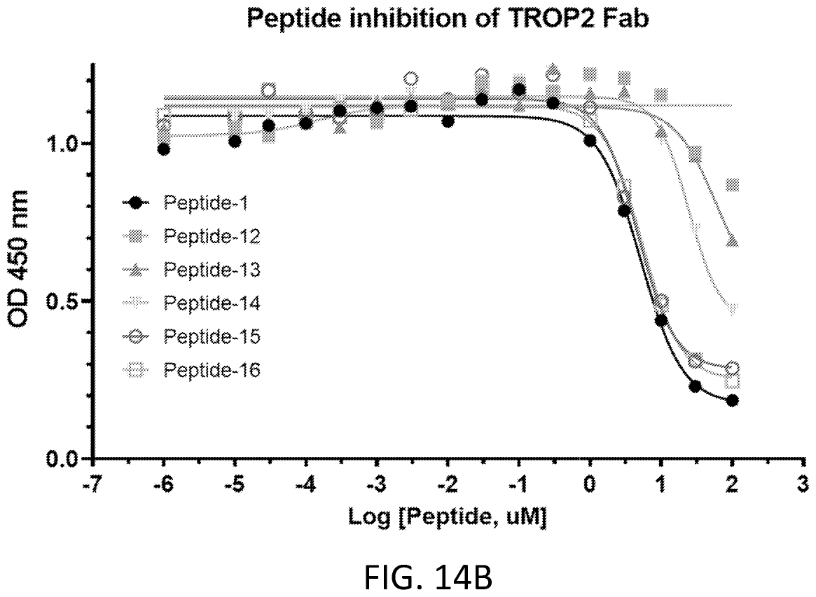

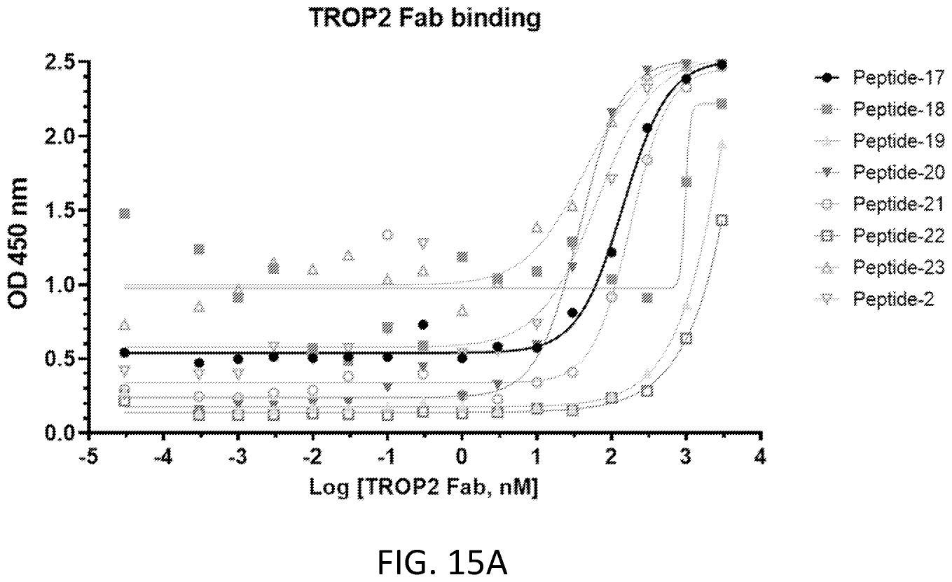

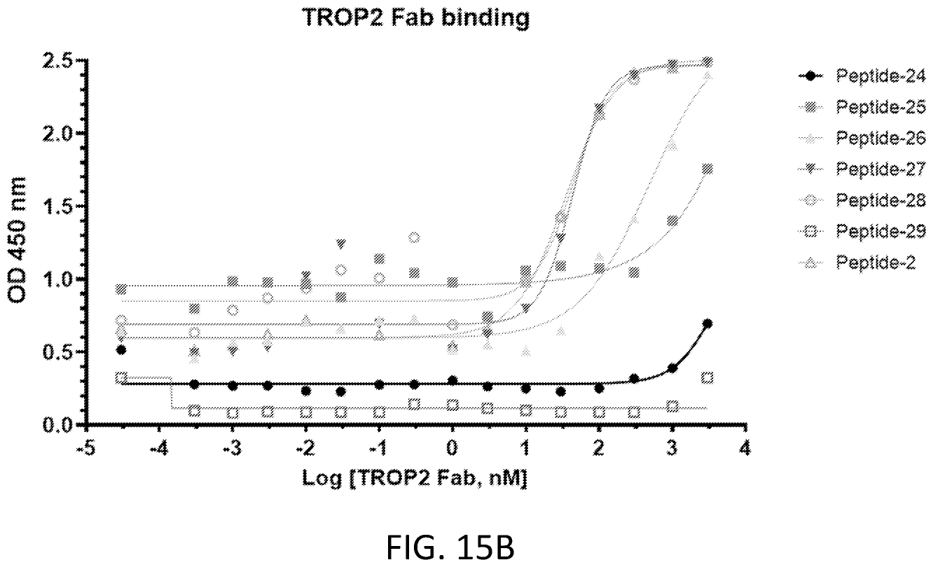

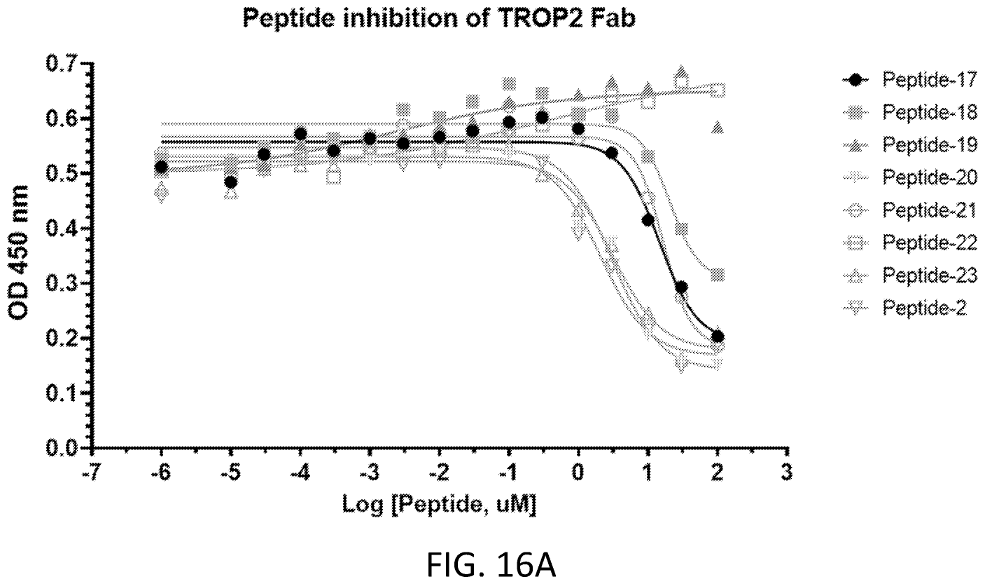

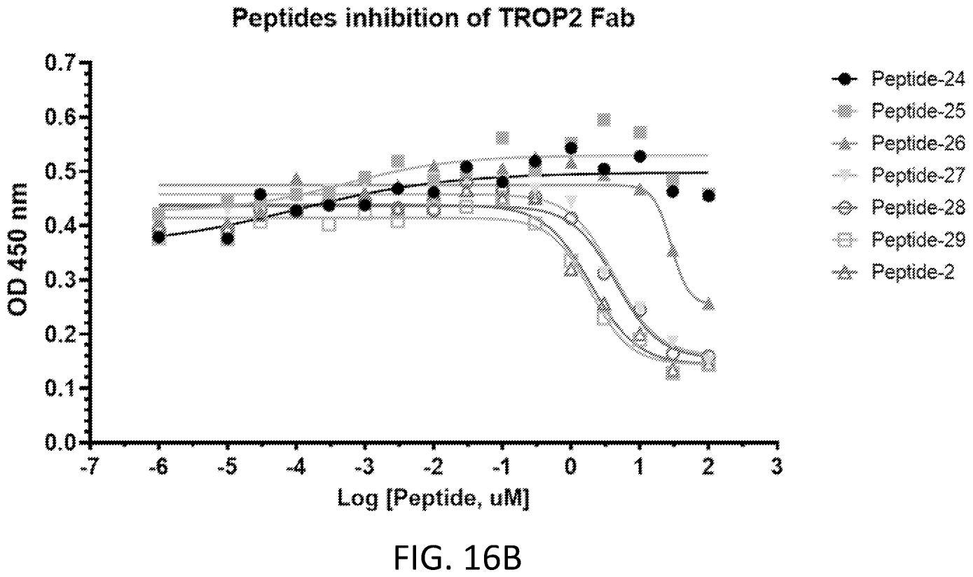

Sequence activity relationships were established for TROP2 Fab Peptide-1 and TROP2 Fab Peptide-2 by mutating each individual residue within the peptide to alanine and measuring binding and inhibition against TROP2 Fab. Peptide residues whose alanine mutations significantly weakened binding and inhibition were considered key residues where mutations were not tolerated. Peptide residues whose alanine mutations performed similarly to the non-mutated sequence were considered non-critical sites where mutations were indeed tolerated. Using the peptide sequence activity relationships (SAR), DNA oligo libraries were constructed where codons encoding critical residues within each peptide sequence were minimally mutated and codons encoding non-critical residues were heavily mutated. The resulting oligos were cloned into bacteriophage vectors used to display the SAR guided peptides via fusion to the pIII filament of the bacteriophage. The relevant vectors were then used to produce the phage optimization libraries via amplification in bacteria using standard techniques in the field. FIG. 14A and FIG. 14B demonstrate TROP2 Fab inhibition of alanine scanning peptides of TROP2 Fab Peptide-1, the sequences of which are shown in Table 25. FIG. 15A and FIG. 15B demonstrate TROP2 Fab binding of alanine scanning peptides of TROP2 Fab Peptide-2. FIG. 16A and FIG. 16B demonstrate TROP2 Fab inhibition of alanine scanning peptides of TROP2 Fab Peptide-2, the sequences of which are shown in Table 26.

TABLE 25
TROP2 Fab Peptide-1 Ala Scan Peptide Sequences

| TROP2 Fab | | |
|---|---|---|
| Peptide-1 Ala | | |
| Scan Peptide | Amino acid sequence | SEQ ID NO: |
| Peptide-4 | AVLFCVKNLYCWVT | 133 |
| Peptide-5 | SALFCVKNLYCWVT | 134 |
| Peptide-6 | SVAFCVKNLYCWVT | 135 |
| Peptide-7 | SVLACVKNLYCWVT | 136 |
| Peptide-8 | SVLFCAKNLYCWVT | 137 |
| Peptide-9 | SVLFCVANLYCWVT | 138 |
| Peptide-10 | SVLFCVKALYCWVT | 139 |
| Peptide-11 | SVLFCVKNAYCWVT | 140 |
| Peptide-12 | SVLFCVKNLACWVT | 141 |
| Peptide-13 | SVLFCVKNLYCAVT | 142 |
| Peptide-14 | SVLFCVKNLYCWAT | 143 |
| Peptide-15 | SVLFCVKNLYCWVA | 144 |
| Peptide-16 | SVLFCVKNLYCWVT | 145 |

TABLE 26
TROP2 Fab Peptide-2 Ala Scan Peptide Sequences

| TROP2 Fab | | |
|---|---|---|
| Peptide-2 Ala | | |
| Scan Peptide | Amino Acid Sequence | SEQ ID NO: |
| Peptide-17 | ADFCKIYSWPVCHQ | 146 |
| Peptide-18 | VAFCKIYSWPVCHQ | 147 |
| Peptide-19 | VDACKIYSWPVCHQ | 148 |
| Peptide-20 | VDFCAIYSWPVCHQ | 149 |
| Peptide-21 | VDFCKAYSWPVCHQ | 150 |
| Peptide-22 | VDFCKIASWPVCHQ | 151 |
| Peptide-23 | VDFCKIYAWPVCHQ | 152 |
| Peptide-24 | VDFCKIYSAPVCHQ | 153 |
| Peptide-25 | VDFCKIYSWAVCHQ | 154 |
| Peptide-26 | VDFCKIYSWPACHQ | 155 |
| Peptide-27 | VDFCKIYSWPVCAQ | 156 |
| Peptide-28 | VDFCKIYSWPVCHA | 157 |
| Peptide-29 | VDFCKIYSWPVCHQ | 158 |

### Example 8: Panning of the Optimized Phage Library—TROP2 Fab Peptides

Once the phage optimization libraries were completed, phage libraries were bio-panned using TROP2 Fab loaded beads. Multiple rounds of panning were performed where bacteriophage was allowed to bind to TROP2 Fab loaded beads, washed, eluted, and amplified. Additional selective pressure was included during each round of panning using a fixed concentration of TROP2 soluble protein, TROP2 Fab Peptide-1, or TROP2 Fab Peptide-2. After panning, phage infected bacteria were plated out and colonies picked into 96 well blocks. Clonal phage was then amplified and separated from bacterial cells via centrifugation. Phage containing supernatants were tested in binding ELISAs against TROP2 Fab coated plates in the presence or absence of saturating concentration of TROP2 soluble protein. Phage able to bind TROP2 Fab were selected for sequence analysis if the binding signal was reduced in the presence of TROP2 soluble protein.

### Example 9: Panning ELISAs—TROP2 Fab Peptides

Clonal phage were harvested as crude supernatants and screened via standard enzyme linked immunosorbent assays (ELISAs). Briefly, biotinylated TROP2 Fab was captured on neutravidin coated plates. Prior to the addition of clonal phage, wells were incubated with blocking buffer and TROP2 soluble protein or blocking buffer alone. Without washing or aspirating, clonal phage supernatants were then added to the wells and incubated for a short time. Wells were then washed followed by detection of bound phage using a horse radish peroxidase conjugated anti-M13 antibody. Clonal phage of interest were then sent for sequence analysis.

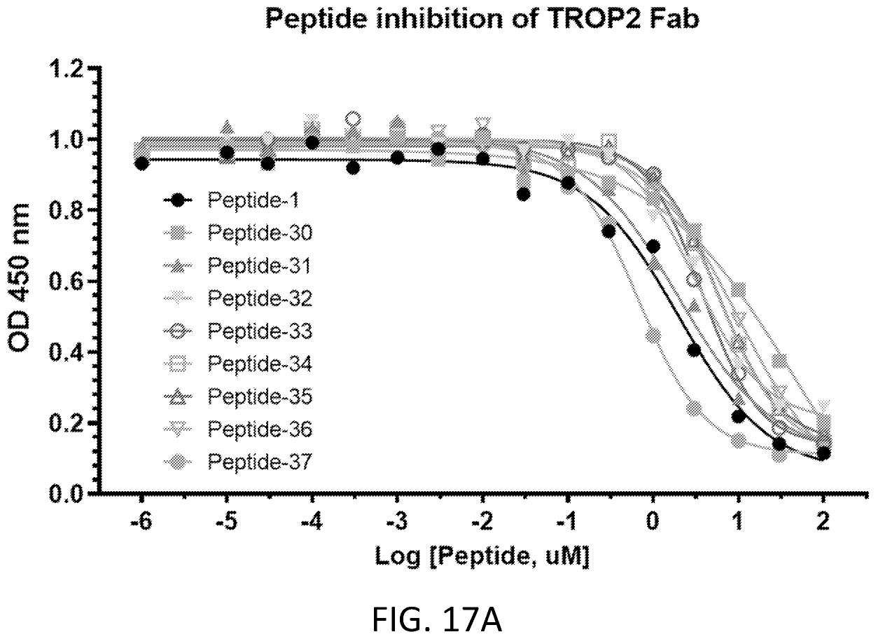

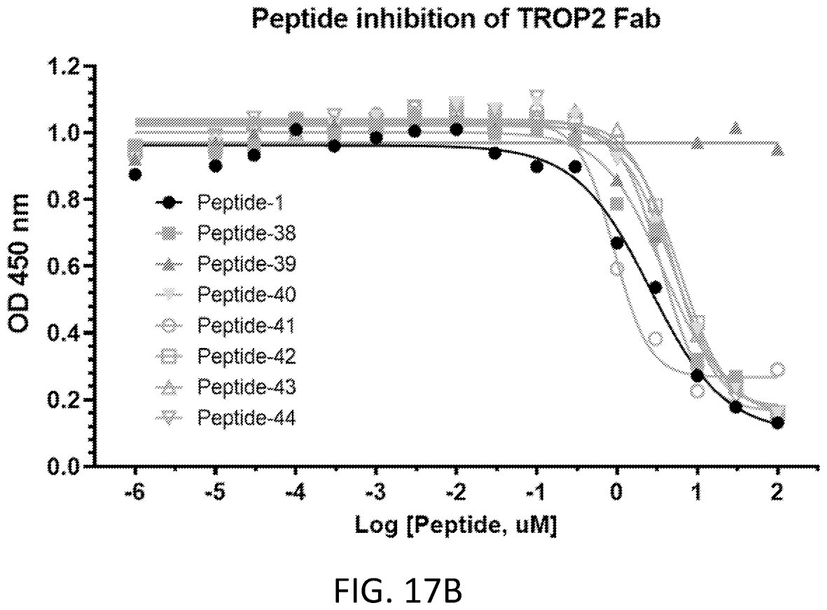

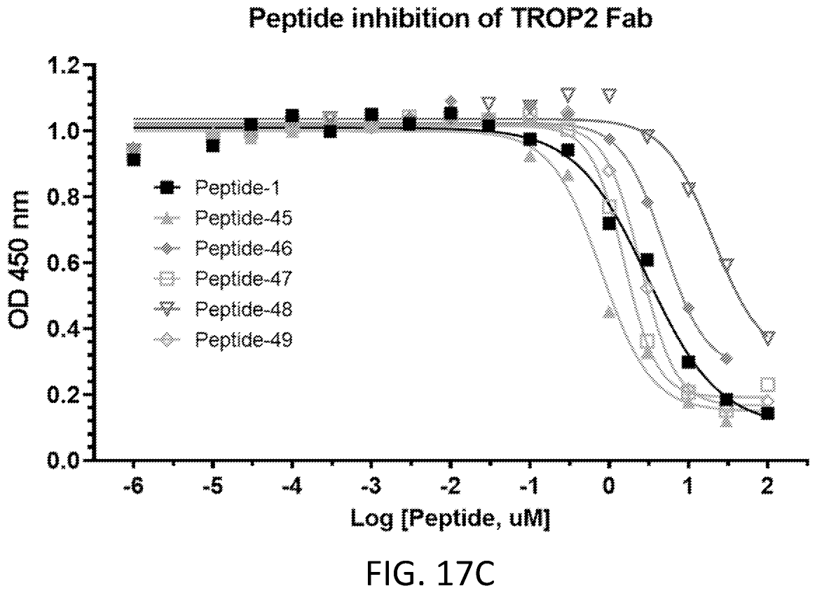

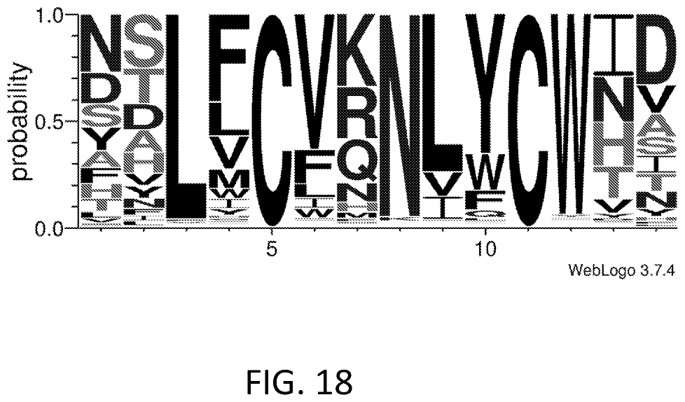

Phage panning results of TROP2 Fab Peptide-1 library sequences are shown in Table 27. 979 clonal phage sequences were identified. 20 of 979 clonal phage were selected for peptide synthesis and evaluated for peptide inhibition of TROP2 Fab. The sequences of those peptides selected for synthesis are shown in Table 28, and further evaluated for inhibition of TROP2 Fab as shown in FIGS. 17A through 17C. The consensus sequence calculated from all the sequences of Table 27 is shown in FIG. 18 and was generated using WebLogo 3.7.4.

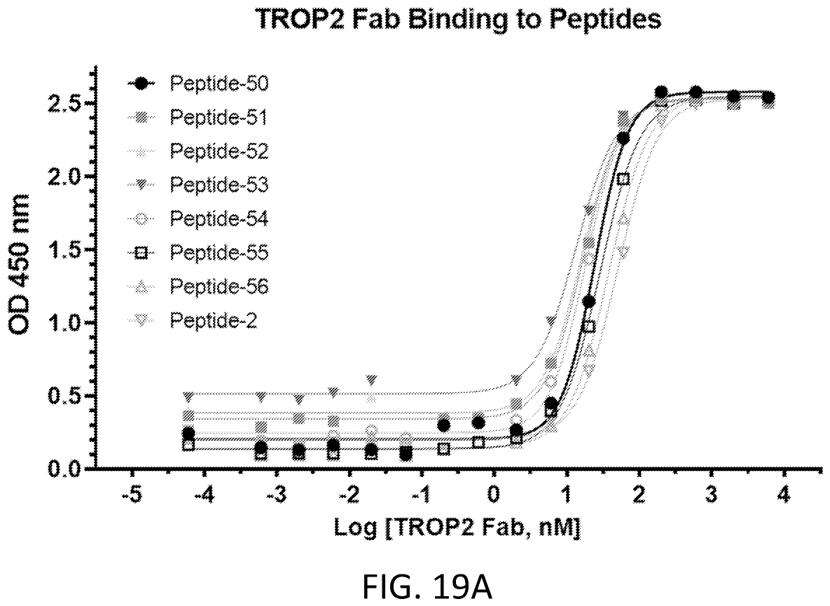

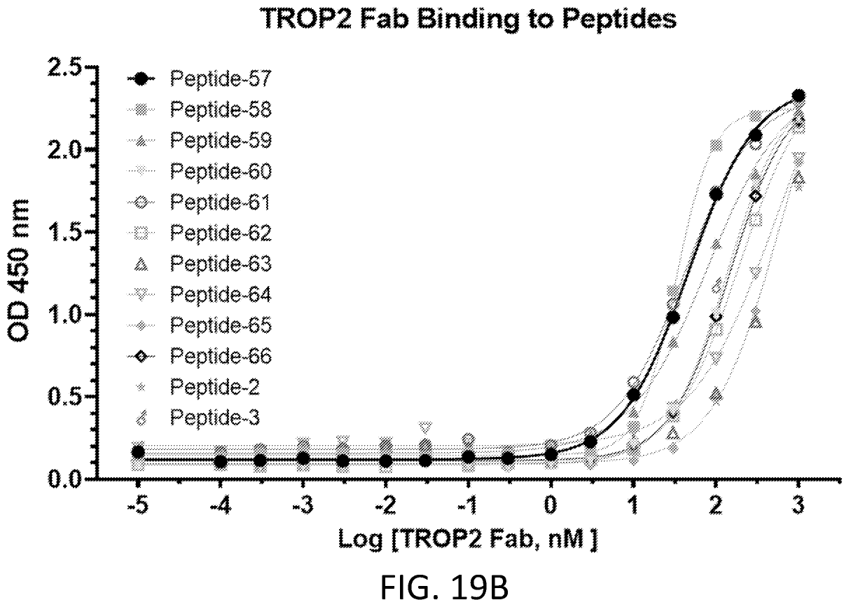

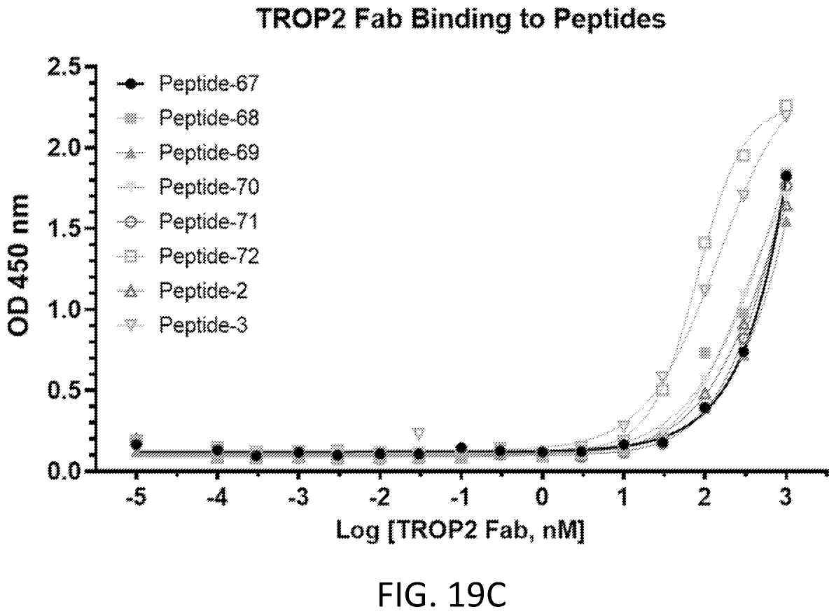

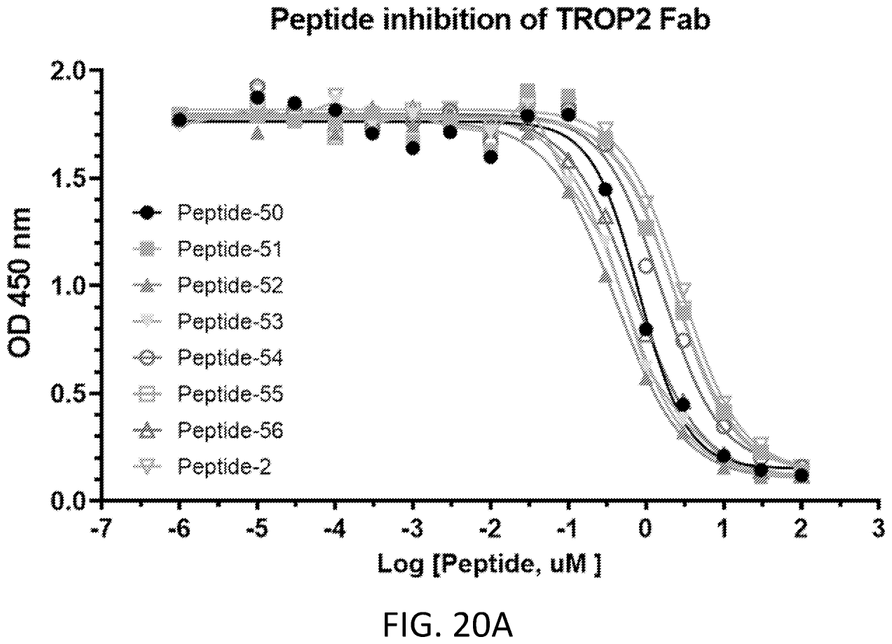

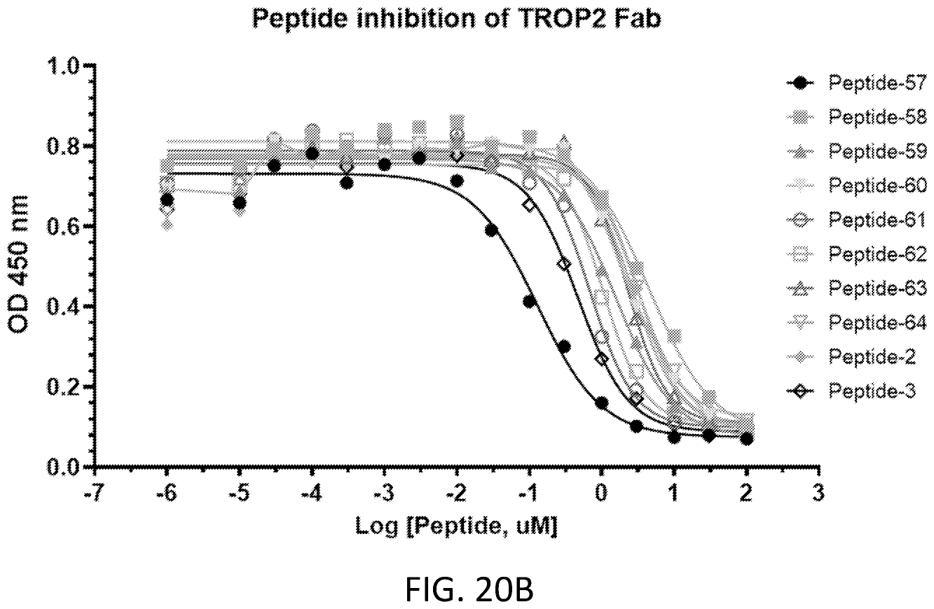

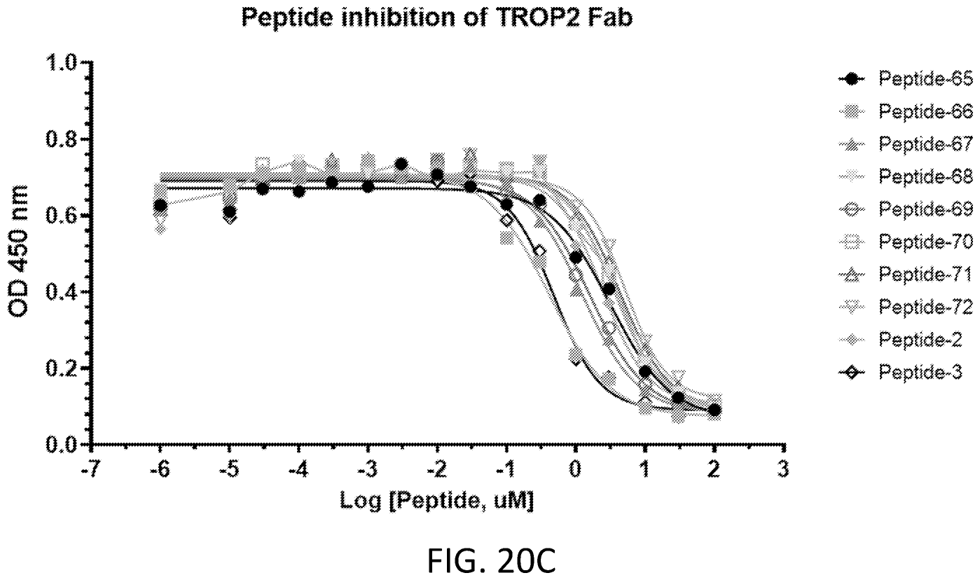

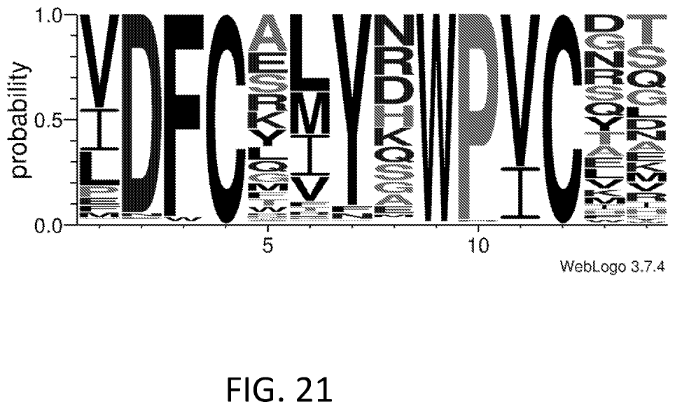

Phage panning results of TROP2 Fab Peptide-2 library sequences are shown in Table 29. 596 clonal phage sequences were identified. 23 of 596 were selected for peptide synthesis and evaluated for peptide binding to TROP2 Fab (FIGS. 19A-19C), and peptide inhibition of TROP2 Fab (FIGS. 20A-20C). The sequences of those peptides selected for synthesis are shown in Table 30. The consensus sequence calculated from all the sequences of Table 29 is shown in FIG. 21 and was generated using WebLogo 3.7.4.

TABLE 27
Phage panning results of TROP2 Fab Peptide-1 library sequences. (—) indicates same amino
acid as in TROP2 Fab Peptide-1 corresponding position (e.g. Phage-1 position).

| | Amino acid position sequence | | | | | | | | | | | | | |
|---|---|---|---|---|---|---|---|---|---|---|---|---|---|---|
| 2 | 1 | 2 | 3 | 4 | 5 | 6 | 7 | 8 | 9 | 10 | 11 | 12 | 13 | 14 |
| Phage-1 | S | V | L | F | C | V | K | N | L | Y | C | W | V | T |
| Phage-2 | D | D | — | I | — | — | — | — | — | F | — | — | T | S |
| Phage-3 | D | N | — | I | — | — | — | — | — | W | — | — | I | A |
| Phage-4 | D | T | — | — | — | — | — | — | — | — | — | — | I | V |
| Phage-5 | D | T | — | S | — | F | R | — | — | — | — | — | I | — |
| Phage-6 | D | T | — | W | — | — | — | — | — | — | — | — | — | A |
| Phage-7 | D | — | — | — | — | — | R | — | — | — | — | — | T | S |
| Phage-8 | F | D | — | L | — | — | R | — | — | — | — | — | N | V |
| Phage-9 | F | S | — | V | — | — | R | — | — | — | — | — | N | V |
| Phage- | F | T | — | — | — | — | Q | — | — | — | — | — | N | V |
| 10 | | | | | | | | | | | | | | |
| Phage- | H | D | — | Q | — | F | Q | — | — | F | — | — | I | V |
| 11 | | | | | | | | | | | | | | |
| Phage- | L | S | — | — | — | — | — | — | — | — | — | — | N | V |
| 12 | | | | | | | | | | | | | | |
| Phage- | N | Y | — | L | — | — | — | — | — | — | — | — | I | V |
| 13 | | | | | | | | | | | | | | |
| Phage- | — | T | — | E | — | — | R | — | — | — | — | — | I | S |
| 14 | | | | | | | | | | | | | | |
| Phage- | — | T | — | — | — | — | — | — | — | — | — | — | — | A |
| 15 | | | | | | | | | | | | | | |
| Phage- | — | T | — | — | — | — | R | — | — | — | — | — | T | V |
| 16 | | | | | | | | | | | | | | |
| Phage- | — | Y | — | V | — | — | — | — | V | — | — | — | T | A |
| 17 | | | | | | | | | | | | | | |
| Phage- | T | A | — | — | — | F | — | — | V | — | — | — | N | V |
| 18 | | | | | | | | | | | | | | |
| Phage- | T | S | — | I | — | F | R | — | V | — | — | — | N | V |
| 19 | | | | | | | | | | | | | | |
| Phage- | Y | D | — | V | — | L | R | — | — | F | — | — | T | A |
| 20 | | | | | | | | | | | | | | |
| Phage- | Y | S | — | V | — | — | — | — | — | — | — | — | N | L |
| 21 | | | | | | | | | | | | | | |
| Phage- | A | T | — | — | — | — | R | — | — | — | — | — | T | A |
| 22 | | | | | | | | | | | | | | |
| Phage- | F | S | — | L | — | — | R | — | — | — | — | — | N | V |
| 23 | | | | | | | | | | | | | | |
| Phage- | N | A | — | V | — | — | R | — | — | — | — | — | I | Y |
| 24 | | | | | | | | | | | | | | |
| Phage- | T | S | — | W | — | — | R | — | — | — | — | Y | N | V |
| 25 | | | | | | | | | | | | | | |
| Phage- | Y | T | — | Y | — | — | Q | — | V | — | — | — | T | L |
| 26 | | | | | | | | | | | | | | |
| Phage- | D | T | — | — | — | — | — | — | — | — | — | — | I | N |
| 27 | | | | | | | | | | | | | | |
| Phage- | Y | S | — | V | — | — | N | — | — | — | — | — | N | — |
| 28 | | | | | | | | | | | | | | |
| Phage- | T | D | — | V | — | — | M | — | R | W | — | — | H | Y |
| 29 | | | | | | | | | | | | | | |
| Phage- | N | T | — | — | — | — | Q | — | — | — | — | — | I | H |
| 30 | | | | | | | | | | | | | | |
| Phage- | N | T | — | — | — | — | R | — | — | — | — | — | I | N |
| 31 | | | | | | | | | | | | | | |
| Phage- | T | D | — | L | — | F | Q | — | I | — | — | — | H | — |
| 32 | | | | | | | | | | | | | | |
| Phage- | N | S | — | — | — | L | H | — | — | V | — | — | I | D |
| 33 | | | | | | | | | | | | | | |
| Phage- | N | T | — | V | — | — | Q | — | V | — | — | — | I | N |
| 34 | | | | | | | | | | | | | | |
| Phage- | N | S | — | — | — | F | Q | — | — | L | — | — | T | D |
| 35 | | | | | | | | | | | | | | |
| Phage- | N | H | — | I | — | — | — | — | — | W | — | — | I | N |
| 36 | | | | | | | | | | | | | | |
| Phage- | — | D | — | — | — | L | — | — | I | — | — | — | H | V |
| 37 | | | | | | | | | | | | | | |
| Phage- | N | I | — | V | — | — | N | — | I | — | — | — | I | D |
| 38 | | | | | | | | | | | | | | |
| Phage- | N | T | — | W | — | — | — | — | — | — | — | — | I | D |
| 39 | | | | | | | | | | | | | | |
| Phage- | D | T | — | — | — | — | — | — | — | F | — | — | T | H |
| 40 | | | | | | | | | | | | | | |
| Phage- | A | T | — | — | — | F | Q | — | — | — | — | — | N | I |
| 41 | | | | | | | | | | | | | | |
| Phage- | Y | A | — | — | — | — | N | — | I | — | — | — | I | S |
| 42 | | | | | | | | | | | | | | |
| Phage- | — | D | — | — | — | — | R | — | — | — | — | — | N | S |
| 43 | | | | | | | | | | | | | | |
| Phage- | D | — | — | I | — | — | — | — | — | — | — | — | I | H |
| 44 | | | | | | | | | | | | | | |
| Phage- | N | T | — | L | — | F | R | — | — | — | — | — | — | D |
| 45 | | | | | | | | | | | | | | |
| Phage- | — | H | — | M | — | — | R | — | — | — | — | — | I | S |
| 46 | | | | | | | | | | | | | | |
| Phage- | D | H | — | V | — | — | — | — | — | — | — | — | I | D |
| 47 | | | | | | | | | | | | | | |
| Phage- | N | S | — | — | — | — | — | — | — | — | — | — | — | D |
| 48 | | | | | | | | | | | | | | |
| Phage- | A | S | — | — | — | — | Q | — | — | W | — | — | T | S |
| 49 | | | | | | | | | | | | | | |
| Phage- | A | S | — | — | — | F | Q | — | — | — | — | — | N | Y |
| 50 | | | | | | | | | | | | | | |
| Phage- | L | S | — | — | — | — | H | — | I | — | — | — | T | D |
| 51 | | | | | | | | | | | | | | |
| Phage- | T | T | — | Y | — | F | Q | — | — | — | — | — | S | V |
| 52 | | | | | | | | | | | | | | |
| Phage- | P | A | — | V | — | — | R | — | — | — | — | — | I | V |
| 53 | | | | | | | | | | | | | | |
| Phage- | N | S | — | — | — | — | Q | — | — | — | — | — | I | I |
| 54 | | | | | | | | | | | | | | |
| Phage- | A | S | — | L | — | — | — | — | — | — | — | — | I | D |
| 55 | | | | | | | | | | | | | | |
| Phage- | N | S | — | — | — | L | — | — | — | — | — | — | I | D |
| 56 | | | | | | | | | | | | | | |
| Phage- | N | S | — | L | — | — | — | — | — | W | — | — | I | D |
| 57 | | | | | | | | | | | | | | |
| Phage- | — | D | — | — | — | W | R | — | — | F | — | — | H | I |
| 58 | | | | | | | | | | | | | | |
| Phage- | N | N | — | V | — | | — | — | — | W | — | — | — | D |
| 59 | | | | | | | | | | | | | | |
| Phage- | Y | H | — | M | — | — | R | — | — | — | — | — | — | V |
| 60 | | | | | | | | | | | | | | |
| Phage- | — | S | — | V | — | — | R | — | — | — | — | — | I | D |
| 61 | | | | | | | | | | | | | | |
| Phage- | T | T | — | L | — | — | — | — | — | — | — | — | H | S |
| 62 | | | | | | | | | | | | | | |
| Phage- | T | S | — | — | — | F | Q | — | V | — | — | — | I | D |
| 63 | | | | | | | | | | | | | | |
| Phage- | T | S | — | I | — | — | R | — | I | L | — | — | H | S |
| 64 | | | | | | | | | | | | | | |
| Phage- | D | — | — | L | — | F | R | — | — | W | — | — | H | — |
| 65 | | | | | | | | | | | | | | |
| Phage- | T | D | — | I | — | — | M | — | — | — | — | — | H | — |
| 66 | | | | | | | | | | | | | | |
| Phage- | L | D | — | I | — | — | R | — | — | — | — | — | I | N |
| 67 | | | | | | | | | | | | | | |
| Phage- | N | Y | — | V | — | E | — | — | — | — | — | R | I | D |
| 68 | | | | | | | | | | | | | | |
| Phage- | N | T | — | I | — | — | R | — | — | — | — | — | — | I |
| 69 | | | | | | | | | | | | | | |
| Phage- | N | A | — | L | — | W | Q | — | I | — | — | — | I | D |
| 70 | | | | | | | | | | | | | | |
| Phage- | N | T | — | L | — | — | N | — | — | W | — | — | I | D |
| 71 | | | | | | | | | | | | | | |
| Phage- | L | S | — | — | — | — | — | — | — | — | — | — | N | A |
| 72 | | | | | | | | | | | | | | |
| Phage- | N | N | — | Y | — | F | Q | — | — | — | — | — | I | D |
| 73 | | | | | | | | | | | | | | |
| Phage- | L | A | — | — | — | — | — | — | I | — | — | — | T | S |
| 74 | | | | | | | | | | | | | | |
| Phage- | T | H | — | V | — | — | Y | — | V | — | — | — | H | A |
| 75 | | | | | | | | | | | | | | |
| Phage- | N | T | — | I | — | L | N | — | — | W | — | — | I | D |
| 76 | | | | | | | | | | | | | | |
| Phage- | N | N | — | L | — | — | R | — | — | — | — | — | I | D |
| 77 | | | | | | | | | | | | | | |
| Phage- | N | T | — | V | — | — | N | — | — | F | — | — | I | N |
| 78 | | | | | | | | | | | | | | |
| Phage- | T | S | — | M | — | — | — | — | — | F | — | — | H | — |
| 79 | | | | | | | | | | | | | | |
| Phage- | — | S | F | S | — | W | H | — | — | — | — | — | D | V |
| 80 | | | | | | | | | | | | | | |
| Phage- | D | A | — | — | — | — | R | — | — | — | — | — | I | D |
| 81 | | | | | | | | | | | | | | |
| Phage- | V | D | — | V | — | I | R | — | — | — | — | — | N | D |
| 82 | | | | | | | | | | | | | | |
| Phage- | — | T | — | — | — | — | — | — | — | — | — | — | T | N |
| 83 | | | | | | | | | | | | | | |
| Phage- | A | N | — | V | — | — | Q | — | — | — | — | — | I | — |
| 84 | | | | | | | | | | | | | | |
| Phage- | A | A | — | — | — | — | — | — | — | — | — | — | I | D |
| 85 | | | | | | | | | | | | | | |
| Phage- | — | S | — | — | — | — | — | — | — | — | — | — | — | D |
| 86 | | | | | | | | | | | | | | |
| Phage- | H | D | — | V | — | F | — | — | — | W | — | — | N | D |
| 87 | | | | | | | | | | | | | | |
| Phage- | N | H | — | L | — | L | N | — | — | — | — | — | I | D |
| 88 | | | | | | | | | | | | | | |
| Phage- | Y | D | — | V | — | — | R | — | — | — | — | — | H | D |
| 89 | | | | | | | | | | | | | | |
| Phage- | — | S | — | V | — | — | N | — | — | — | — | — | N | — |
| 90 | | | | | | | | | | | | | | |
| Phage- | — | D | — | — | — | — | M | — | — | — | — | — | H | Y |
| 91 | | | | | | | | | | | | | | |
| Phage- | D | S | — | L | — | F | Q | — | — | W | — | — | I | V |
| 92 | | | | | | | | | | | | | | |
| Phage- | N | H | — | L | — | — | R | — | — | — | — | — | — | — |
| 93 | | | | | | | | | | | | | | |
| Phage- | A | D | — | — | — | — | — | — | — | W | — | — | H | S |
| 94 | | | | | | | | | | | | | | |
| Phage- | N | A | — | — | — | F | N | — | — | — | — | — | I | D |
| 95 | | | | | | | | | | | | | | |
| Phage- | N | S | — | — | — | — | Q | — | V | — | — | — | N | — |
| 96 | | | | | | | | | | | | | | |
| Phage- | T | N | — | M | — | — | R | — | — | — | — | — | N | D |
| 97 | | | | | | | | | | | | | | |
| Phage- | I | H | — | I | — | — | E | — | I | — | — | — | H | V |
| 98 | | | | | | | | | | | | | | |
| Phage- | A | D | — | L | — | — | R | — | — | — | — | — | H | D |
| 99 | | | | | | | | | | | | | | |
| Phage- | H | S | — | — | — | — | — | — | — | — | — | — | H | — |
| 100 | | | | | | | | | | | | | | |
| Phage- | — | — | — | — | — | — | R | — | — | — | — | — | T | D |
| 101 | | | | | | | | | | | | | | |
| Phage- | T | N | — | — | — | — | R | — | — | — | — | — | T | D |
| 102 | | | | | | | | | | | | | | |
| Phage- | — | T | — | Y | — | — | — | — | — | — | — | — | T | D |
| 103 | | | | | | | | | | | | | | |
| Phage- | N | H | — | L | — | — | D | — | — | W | — | — | I | D |
| 104 | | | | | | | | | | | | | | |
| Phage- | D | A | — | L | — | W | R | — | — | — | — | — | T | N |
| 105 | | | | | | | | | | | | | | |
| Phage- | D | H | — | V | — | — | Q | — | — | F | — | — | I | D |
| 106 | | | | | | | | | | | | | | |
| Phage- | A | T | — | S | — | F | Q | — | — | — | — | — | H | I |
| 107 | | | | | | | | | | | | | | |
| Phage- | — | N | — | — | — | — | — | — | — | F | — | — | T | D |
| 108 | | | | | | | | | | | | | | |
| Phage- | D | T | — | — | — | — | — | — | — | F | — | — | I | — |
| 109 | | | | | | | | | | | | | | |
| Phage- | — | — | — | W | — | — | R | — | — | — | — | — | N | D |
| 110 | | | | | | | | | | | | | | |
| Phage- | D | H | — | M | — | — | — | — | — | W | — | — | — | D |
| 111 | | | | | | | | | | | | | | |
| Phage- | N | T | — | — | — | M | D | — | I | W | — | — | H | D |
| 112 | | | | | | | | | | | | | | |
| Phage- | V | S | — | L | — | — | — | — | — | W | — | — | H | I |
| 113 | | | | | | | | | | | | | | |
| Phage- | H | S | — | — | — | F | — | — | — | — | — | — | N | V |
| 114 | | | | | | | | | | | | | | |
| Phage- | D | H | — | — | — | — | Q | — | — | W | — | — | I | S |
| 115 | | | | | | | | | | | | | | |
| Phage- | — | N | — | V | — | — | N | — | — | W | — | — | I | D |
| 116 | | | | | | | | | | | | | | |
| Phage- | N | H | — | V | — | — | N | — | — | W | — | — | H | D |
| 117 | | | | | | | | | | | | | | |
| Phage- | T | N | — | V | — | — | N | — | — | W | — | — | L | D |
| 118 | | | | | | | | | | | | | | |
| Phage- | D | T | — | — | — | — | R | — | — | — | — | — | I | D |
| 119 | | | | | | | | | | | | | | |
| Phage- | N | A | — | L | — | L | — | — | — | — | — | — | I | D |
| 120 | | | | | | | | | | | | | | |
| Phage- | T | S | — | E | — | W | — | — | — | W | — | — | H | S |
| 121 | | | | | | | | | | | | | | |
| Phage- | — | H | — | — | — | — | — | — | V | F | — | — | D | D |
| 122 | | | | | | | | | | | | | | |
| Phage- | N | A | — | W | — | I | Q | — | — | — | — | Y | T | D |
| 123 | | | | | | | | | | | | | | |
| Phage- | N | S | — | L | — | F | Q | — | — | F | — | — | H | S |
| 124 | | | | | | | | | | | | | | |
| Phage- | — | D | — | V | — | — | — | — | — | — | — | — | N | I |
| 125 | | | | | | | | | | | | | | |
| Phage- | H | S | — | W | — | I | N | — | — | — | — | — | H | D |
| 126 | | | | | | | | | | | | | | |
| Phage- | Y | A | — | — | — | — | — | — | — | W | — | — | N | A |
| 127 | | | | | | | | | | | | | | |
| Phage- | A | H | — | W | — | W | — | — | — | W | — | — | — | D |
| 128 | | | | | | | | | | | | | | |
| Phage- | D | Y | — | M | — | F | R | — | — | W | — | — | H | S |
| 129 | | | | | | | | | | | | | | |
| Phage- | — | — | W | L | — | M | L | K | — | Q | — | F | Y | N |
| 130 | | | | | | | | | | | | | | |
| Phage- | Y | H | F | M | — | — | N | D | F | D | — | — | F | I |
| 131 | | | | | | | | | | | | | | |
| Phage- | Y | H | F | L | — | L | N | E | — | — | — | — | A | I |
| 132 | | | | | | | | | | | | | | |
| Phage- | N | — | — | — | — | Y | H | — | V | — | — | — | — | N |
| 133 | | | | | | | | | | | | | | |
| Phage- | N | F | E | L | — | L | — | — | — | Q | — | S | F | A |
| 134 | | | | | | | | | | | | | | |
| Phage- | Y | H | F | L | — | M | N | D | — | E | — | — | Y | I |
| 135 | | | | | | | | | | | | | | |
| Phage- | N | Y | V | L | — | L | T | K | — | P | — | — | — | I |
| 136 | | | | | | | | | | | | | | |
| Phage- | A | Y | — | V | — | — | — | — | — | F | — | — | T | H |
| 137 | | | | | | | | | | | | | | |
| Phage- | Y | D | — | L | — | — | — | Q | — | Q | — | R | Y | H |
| 138 | | | | | | | | | | | | | | |
| Phage- | D | L | — | M | — | — | R | — | — | — | — | — | I | N |
| 139 | | | | | | | | | | | | | | |
| Phage- | F | D | F | L | — | F | D | E | W | Q | — | L | Y | L |
| 140 | | | | | | | | | | | | | | |
| Phage- | D | F | — | V | — | — | R | — | — | — | | — | I | D |
| 141 | | | | | | | | | | | | | | |
| Phage- | L | H | R | L | — | F | E | — | F | Q | — | R | N | Y |
| 142 | | | | | | | | | | | | | | |
| Phage- | F | N | — | M | — | A | Q | D | R | D | — | A | S | D |
| 143 | | | | | | | | | | | | | | |
| Phage- | — | A | — | — | — | F | Q | — | V | — | — | — | H | S |
| 144 | | | | | | | | | | | | | | |
| Phage- | A | D | — | — | — | — | — | — | — | W | — | — | T | S |
| 145 | | | | | | | | | | | | | | |
| Phage- | H | S | — | — | — | — | — | — | — | F | — | — | N | D |
| 146 | | | | | | | | | | | | | | |
| Phage- | N | F | — | V | — | — | Q | — | — | — | — | — | I | D |
| 147 | | | | | | | | | | | | | | |
| Phage- | F | S | — | M | — | — | Q | — | — | — | — | — | H | V |
| 148 | | | | | | | | | | | | | | |
| Phage- | N | A | — | L | — | L | N | H | — | Q | — | — | H | — |
| 149 | | | | | | | | | | | | | | |
| Phage- | A | H | — | L | — | — | Q | R | P | Q | — | R | — | D |
| 150 | | | | | | | | | | | | | | |
| Phage- | — | D | — | — | — | L | Q | — | V | — | — | — | H | — |
| 151 | | | | | | | | | | | | | | |
| Phage- | — | T | V | Q | — | I | E | T | P | Q | — | L | I | H |
| 152 | | | | | | | | | | | | | | |
| Phage- | V | — | — | L | — | — | N | — | V | M | — | — | P | V |
| 153 | | | | | | | | | | | | | | |
| Phage- | D | S | — | V | — | — | — | — | — | — | — | — | I | N |
| 154 | | | | | | | | | | | | | | |
| Phage- | V | F | — | — | — | — | E | — | H | Q | — | S | — | Y |
| 155 | | | | | | | | | | | | | | |
| Phage- | D | S | — | L | — | — | — | R | R | Q | — | — | H | L |
| 156 | | | | | | | | | | | | | | |
| Phage- | F | — | — | — | — | G | N | D | Q | Q | — | — | A | S |
| 157 | | | | | | | | | | | | | | |
| Phage- | I | P | R | L | — | L | H | K | — | W | — | G | N | S |
| 158 | | | | | | | | | | | | | | |
| Phage- | V | S | — | M | — | — | — | — | V | F | — | — | Y | A |
| 159 | | | | | | | | | | | | | | |
| Phage- | H | T | — | L | — | F | H | — | — | W | — | — | H | D |
| 160 | | | | | | | | | | | | | | |
| Phage- | N | A | — | W | — | — | N | — | — | — | — | — | — | S |
| 161 | | | | | | | | | | | | | | |
| Phage- | I | T | — | Q | — | — | Q | Q | — | S | — | S | L | V |
| 162 | | | | | | | | | | | | | | |
| Phage- | N | I | — | V | — | — | N | — | — | W | — | — | I | D |
| 163 | | | | | | | | | | | | | | |
| Phage- | P | S | — | — | — | F | Q | — | — | — | — | — | I | D |
| 164 | | | | | | | | | | | | | | |
| Phage- | D | Y | V | L | — | — | G | S | — | P | — | — | P | I |
| 165 | | | | | | | | | | | | | | |
| Phage- | Y | Y | F | L | — | F | S | S | — | N | — | V | Y | V |
| 166 | | | | | | | | | | | | | | |
| Phage- | T | N | F | L | — | G | — | — | — | Q | — | L | I | A |
| 167 | | | | | | | | | | | | | | |
| Phage- | H | Y | P | L | — | — | S | — | — | Q | — | L | — | Y |
| 168 | | | | | | | | | | | | | | |
| Phage- | N | H | — | L | — | — | Q | — | — | — | — | — | Y | D |
| 169 | | | | | | | | | | | | | | |
| Phage- | D | S | V | — | — | — | — | K | — | Q | — | — | D | N |
| 170 | | | | | | | | | | | | | | |
| Phage- | — | — | V | L | — | — | L | — | G | Q | — | F | S | A |
| 171 | | | | | | | | | | | | | | |
| Phage- | V | D | — | S | — | L | — | K | — | Q | — | L | N | A |
| 172 | | | | | | | | | | | | | | |
| Phage- | I | H | R | L | — | T | — | I | R | — | — | F | Y | Y |
| 173 | | | | | | | | | | | | | | |
| Phage- | D | Y | — | — | — | — | — | — | — | — | — | — | H | N |
| 174 | | | | | | | | | | | | | | |
| Phage- | F | T | Q | — | — | A | R | K | M | Q | — | Q | D | N |
| 175 | | | | | | | | | | | | | | |
| Phage- | I | — | — | — | — | G | R | — | — | — | — | — | Y | A |
| 176 | | | | | | | | | | | | | | |
| Phage- | L | Y | — | G | — | R | R | Q | — | L | — | D | D | V |
| 177 | | | | | | | | | | | | | | |
| Phage- | Y | T | — | L | — | A | — | K | Q | H | — | S | A | N |
| 178 | | | | | | | | | | | | | | |
| Phage- | H | — | V | L | — | T | S | E | — | Q | — | Q | Y | S |
| 179 | | | | | | | | | | | | | | |
| Phage- | D | T | — | M | — | I | — | — | — | — | — | — | I | D |
| 180 | | | | | | | | | | | | | | |
| Phage- | — | D | — | L | — | — | R | — | — | — | — | — | I | Y |
| 181 | | | | | | | | | | | | | | |
| Phage- | Y | S | — | M | — | I | Q | — | — | — | — | — | N | D |
| 182 | | | | | | | | | | | | | | |
| Phage- | N | T | — | — | — | — | A | — | — | — | — | — | I | N |
| 183 | | | | | | | | | | | | | | |
| Phage- | — | F | — | — | — | — | Q | — | I | — | — | — | H | N |
| 184 | | | | | | | | | | | | | | |
| Phage- | Y | S | — | Y | — | — | R | — | — | — | — | — | S | D |
| 185 | | | | | | | | | | | | | | |
| Phage- | D | T | — | — | — | — | R | — | — | — | — | — | I | S |
| 186 | | | | | | | | | | | | | | |
| Phage- | Y | D | — | V | — | — | H | — | I | — | — | — | N | F |
| 187 | | | | | | | | | | | | | | |
| Phage- | D | — | — | K | — | — | — | — | — | W | — | — | I | D |
| 188 | | | | | | | | | | | | | | |
| Phage- | N | D | — | — | — | F | Q | — | — | — | — | — | T | D |
| 189 | | | | | | | | | | | | | | |
| Phage- | N | A | — | — | — | — | R | — | — | — | — | — | — | N |
| 190 | | | | | | | | | | | | | | |
| Phage- | — | N | — | L | — | I | — | — | — | — | — | — | H | D |
| 191 | | | | | | | | | | | | | | |
| Phage- | T | T | — | Y | — | — | R | — | — | — | — | — | H | — |
| 192 | | | | | | | | | | | | | | |
| Phage- | A | — | — | V | — | F | Q | — | — | — | — | — | I | D |
| 193 | | | | | | | | | | | | | | |
| Phage- | F | D | — | — | — | — | Q | — | — | — | — | — | N | — |
| 194 | | | | | | | | | | | | | | |
| Phage- | P | T | — | L | — | — | Q | — | — | F | — | — | I | A |
| 195 | | | | | | | | | | | | | | |
| Phage- | H | T | — | — | — | — | Q | — | — | — | — | — | I | D |
| 196 | | | | | | | | | | | | | | |
| Phage- | A | T | — | — | — | — | R | — | — | — | — | — | I | D |
| 197 | | | | | | | | | | | | | | |
| Phage- | N | S | — | — | — | L | — | — | — | — | — | — | — | D |
| 198 | | | | | | | | | | | | | | |
| Phage- | T | N | — | V | — | — | — | — | — | W | — | — | H | D |
| 199 | | | | | | | | | | | | | | |
| Phage- | D | — | — | Q | — | I | — | — | — | — | — | — | I | D |
| 200 | | | | | | | | | | | | | | |
| Phage- | N | — | — | — | — | L | — | — | — | E | — | — | I | D |
| 201 | | | | | | | | | | | | | | |
| Phage- | D | T | — | Y | — | — | — | — | — | W | — | — | I | D |
| 202 | | | | | | | | | | | | | | |
| Phage- | N | T | — | — | — | — | — | — | — | — | — | — | T | D |
| 203 | | | | | | | | | | | | | | |
| Phage- | T | T | — | W | — | — | Q | — | — | — | — | — | H | L |
| 204 | | | | | | | | | | | | | | |
| Phage- | A | L | — | M | — | — | N | — | — | W | — | — | H | D |
| 205 | | | | | | | | | | | | | | |
| Phage- | N | T | — | — | — | — | Q | — | — | F | — | — | — | D |
| 206 | | | | | | | | | | | | | | |
| Phage- | N | S | — | V | — | — | — | — | — | — | — | — | — | D |
| 207 | | | | | | | | | | | | | | |
| Phage- | N | S | — | — | — | — | Q | — | — | — | — | — | I | D |
| 208 | | | | | | | | | | | | | | |
| Phage- | N | A | — | L | — | — | — | — | V | — | — | — | I | A |
| 209 | | | | | | | | | | | | | | |
| Phage- | N | D | — | V | — | — | M | — | — | W | — | — | N | D |
| 210 | | | | | | | | | | | | | | |
| Phage- | Y | D | — | — | — | — | N | — | — | W | — | — | H | D |
| 211 | | | | | | | | | | | | | | |
| Phage- | D | T | — | L | — | — | — | — | — | F | — | Y | I | I |
| 212 | | | | | | | | | | | | | | |
| Phage- | N | S | — | — | — | I | H | — | — | — | — | — | I | D |
| 213 | | | | | | | | | | | | | | |
| Phage- | A | S | — | M | — | — | N | — | — | W | — | — | H | — |
| 214 | | | | | | | | | | | | | | |
| Phage- | Y | A | — | V | — | — | Q | — | I | — | — | — | N | Y |
| 215 | | | | | | | | | | | | | | |
| Phage- | D | S | — | I | — | — | R | — | — | — | — | — | H | Y |
| 216 | | | | | | | | | | | | | | |
| Phage- | N | — | — | I | — | — | N | — | V | — | — | — | I | D |
| 217 | | | | | | | | | | | | | | |
| Phage- | N | T | — | — | — | — | — | — | — | — | — | — | T | Y |
| 218 | | | | | | | | | | | | | | |
| Phage- | N | — | — | L | — | I | D | — | — | — | — | — | I | D |
| 219 | | | | | | | | | | | | | | |
| Phage- | T | D | — | — | — | — | — | — | — | W | — | — | H | N |
| 220 | | | | | | | | | | | | | | |
| Phage- | A | S | — | M | — | — | R | — | — | — | — | — | N | D |
| 221 | | | | | | | | | | | | | | |
| Phage- | — | D | — | — | — | — | Q | — | V | — | — | — | H | H |
| 222 | | | | | | | | | | | | | | |
| Phage- | — | F | — | Y | — | — | Q | — | I | — | — | — | T | V |
| 223 | | | | | | | | | | | | | | |
| Phage- | A | S | — | — | — | — | R | — | — | — | — | — | N | D |
| 224 | | | | | | | | | | | | | | |
| Phage- | N | — | — | L | — | — | — | — | — | — | — | — | N | D |
| 225 | | | | | | | | | | | | | | |
| Phage- | — | I | — | L | — | — | — | — | — | — | — | — | H | V |
| 226 | | | | | | | | | | | | | | |
| Phage- | A | H | — | V | — | — | — | — | — | — | — | — | I | S |
| 227 | | | | | | | | | | | | | | |
| Phage- | H | A | — | — | — | — | — | — | — | — | — | — | N | D |
| 228 | | | | | | | | | | | | | | |
| Phage- | A | T | — | L | — | — | Q | — | V | F | — | — | H | S |
| 229 | | | | | | | | | | | | | | |
| Phage- | F | S | — | V | — | — | N | — | — | W | — | — | I | D |
| 230 | | | | | | | | | | | | | | |
| Phage- | H | A | — | — | — | — | Q | — | — | — | — | — | T | D |
| 231 | | | | | | | | | | | | | | |
| Phage- | Y | D | — | V | — | — | R | — | — | — | — | — | N | I |
| 232 | | | | | | | | | | | | | | |
| Phage- | N | S | — | V | — | — | M | — | V | — | — | — | H | I |
| 233 | | | | | | | | | | | | | | |
| Phage- | N | L | — | V | — | — | — | — | — | — | — | — | I | D |
| 234 | | | | | | | | | | | | | | |
| Phage- | — | S | — | — | — | L | R | — | — | — | — | — | N | A |
| 235 | | | | | | | | | | | | | | |
| Phage- | A | T | — | M | — | — | N | — | I | H | — | — | I | A |
| 236 | | | | | | | | | | | | | | |
| Phage- | T | — | — | R | — | F | R | K | D | Q | — | I | F | S |
| 237 | | | | | | | | | | | | | | |
| Phage- | N | S | — | H | — | I | Q | — | — | — | — | — | I | I |
| 238 | | | | | | | | | | | | | | |
| Phage- | D | I | — | W | — | — | R | — | — | — | — | — | H | I |
| 239 | | | | | | | | | | | | | | |
| Phage- | T | S | — | — | — | L | Q | — | — | — | — | — | H | D |
| 240 | | | | | | | | | | | | | | |
| Phage- | F | T | — | — | — | — | Q | — | — | — | — | — | N | — |
| 241 | | | | | | | | | | | | | | |
| Phage- | — | S | — | Y | — | F | — | — | — | — | — | — | T | I |
| 242 | | | | | | | | | | | | | | |
| Phage- | N | T | — | I | — | F | — | — | — | — | — | — | I | A |
| 243 | | | | | | | | | | | | | | |
| Phage- | N | S | — | L | — | — | Q | — | I | — | — | — | I | D |
| 244 | | | | | | | | | | | | | | |
| Phage- | D | H | — | V | — | — | N | — | — | L | — | — | I | A |
| 245 | | | | | | | | | | | | | | |
| Phage- | — | S | — | — | — | — | Q | — | — | — | — | — | T | D |
| 246 | | | | | | | | | | | | | | |
| Phage- | V | S | — | — | — | L | E | — | I | — | — | — | H | N |
| 247 | | | | | | | | | | | | | | |
| Phage- | A | T | — | — | — | L | Q | — | — | — | — | — | I | S |
| 248 | | | | | | | | | | | | | | |
| Phage- | H | D | — | M | — | — | Q | — | I | — | — | — | N | I |
| 249 | | | | | | | | | | | | | | |
| Phage- | L | L | C | V | Q | N | L | Y | C | W | N | T | G | G |
| 250 | | | | | | | | | | | | | | |
| Phage- | N | S | — | V | — | — | N | — | V | — | — | — | — | V |
| 251 | | | | | | | | | | | | | | |
| Phage- | N | F | — | L | — | — | — | — | V | — | — | — | I | D |
| 252 | | | | | | | | | | | | | | |
| Phage- | — | A | — | — | — | — | Q | — | — | — | — | — | T | D |
| 253 | | | | | | | | | | | | | | |
| Phage- | L | D | — | H | — | — | Q | — | V | — | — | — | I | S |
| 254 | | | | | | | | | | | | | | |
| Phage- | D | T | — | — | — | — | R | — | — | — | — | — | I | — |
| 255 | | | | | | | | | | | | | | |
| Phage- | F | S | — | — | — | — | N | — | — | — | — | — | T | D |
| 256 | | | | | | | | | | | | | | |
| Phage- | D | — | — | V | — | — | — | — | — | — | — | — | I | H |
| 257 | | | | | | | | | | | | | | |
| Phage- | — | D | — | — | — | F | Q | — | — | — | — | — | H | D |
| 258 | | | | | | | | | | | | | | |
| Phage- | — | Y | — | V | — | — | Q | — | V | — | — | — | — | A |
| 259 | | | | | | | | | | | | | | |
| Phage- | T | N | — | M | — | — | R | — | — | — | — | — | H | V |
| 260 | | | | | | | | | | | | | | |
| Phage- | N | L | — | V | — | F | R | — | V | — | — | — | H | N |
| 261 | | | | | | | | | | | | | | |
| Phage- | N | T | — | L | — | — | Q | — | — | — | — | — | I | D |
| 262 | | | | | | | | | | | | | | |
| Phage- | N | L | — | — | — | — | N | — | — | W | — | — | N | D |
| 263 | | | | | | | | | | | | | | |
| Phage- | D | A | — | W | — | Y | Q | — | I | — | — | — | I | D |
| 264 | | | | | | | | | | | | | | |
| Phage- | H | Y | — | L | — | — | — | — | V | — | — | — | T | V |
| 265 | | | | | | | | | | | | | | |
| Phage- | D | S | — | — | — | F | — | — | V | — | — | — | L | A |
| 266 | | | | | | | | | | | | | | |
| Phage- | V | S | — | V | — | — | — | — | I | — | — | — | H | D |
| 267 | | | | | | | | | | | | | | |
| Phage- | L | S | — | — | — | — | — | — | — | — | — | — | T | D |
| 268 | | | | | | | | | | | | | | |
| Phage- | N | S | — | — | — | — | R | — | I | — | — | — | H | D |
| 269 | | | | | | | | | | | | | | |
| Phage- | — | H | — | — | — | — | Q | — | I | — | — | — | — | I |
| 270 | | | | | | | | | | | | | | |
| Phage- | N | T | — | M | — | F | E | — | — | — | — | — | I | D |
| 271 | | | | | | | | | | | | | | |
| Phage- | F | S | — | — | — | — | E | — | V | — | — | — | N | D |
| 272 | | | | | | | | | | | | | | |
| Phage- | N | A | — | — | — | W | Q | — | — | — | — | — | N | D |
| 273 | | | | | | | | | | | | | | |
| Phage- | N | — | — | R | — | — | Q | — | — | — | — | — | I | D |
| 274 | | | | | | | | | | | | | | |
| Phage- | Y | S | — | L | — | F | Q | — | M | — | — | — | N | V |
| 275 | | | | | | | | | | | | | | |
| Phage- | T | H | — | V | — | — | R | — | — | — | — | — | — | I |
| 276 | | | | | | | | | | | | | | |
| Phage- | F | S | — | Y | — | F | Q | — | — | — | — | — | H | D |
| 277 | | | | | | | | | | | | | | |
| Phage- | N | S | — | — | — | — | — | — | V | — | — | — | N | D |
| 278 | | | | | | | | | | | | | | |
| Phage- | D | H | — | L | — | — | N | — | — | W | — | — | I | S |
| 279 | | | | | | | | | | | | | | |
| Phage- | N | S | — | — | — | — | — | — | — | — | — | — | I | I |
| 280 | | | | | | | | | | | | | | |
| Phage- | T | D | — | V | — | — | — | — | — | — | — | — | H | S |
| 281 | | | | | | | | | | | | | | |
| Phage- | F | A | — | Y | — | I | E | — | V | H | — | — | H | I |
| 282 | | | | | | | | | | | | | | |
| Phage- | A | A | — | I | — | I | — | — | — | W | — | — | H | A |
| 283 | | | | | | | | | | | | | | |
| Phage- | H | T | — | V | — | — | — | — | — | — | — | — | I | N |
| 284 | | | | | | | | | | | | | | |
| Phage- | Y | H | — | — | — | — | — | — | — | — | — | — | N | V |
| 285 | | | | | | | | | | | | | | |
| Phage- | D | L | — | — | — | F | R | — | — | W | — | — | N | N |
| 286 | | | | | | | | | | | | | | |
| Phage- | Y | D | — | — | — | — | R | — | — | — | — | — | H | D |
| 287 | | | | | | | | | | | | | | |
| Phage- | N | A | — | L | — | F | Q | — | I | — | — | — | — | D |
| 288 | | | | | | | | | | | | | | |
| Phage- | D | — | — | V | — | L | R | — | — | F | — | — | — | D |
| 289 | | | | | | | | | | | | | | |
| Phage- | Y | H | — | W | — | — | — | — | V | — | — | — | N | D |
| 290 | | | | | | | | | | | | | | |
| Phage- | N | T | — | — | — | — | H | — | — | — | — | — | H | V |
| 291 | | | | | | | | | | | | | | |
| Phage- | N | I | — | L | — | — | Q | — | — | — | — | — | I | D |
| 292 | | | | | | | | | | | | | | |
| Phage- | N | A | — | — | — | — | R | — | — | — | — | — | N | A |
| 293 | | | | | | | | | | | | | | |
| Phage- | N | A | — | V | — | — | H | — | — | — | — | — | I | S |
| 294 | | | | | | | | | | | | | | |
| Phage- | Y | D | — | I | — | — | R | — | — | — | — | — | T | A |
| 295 | | | | | | | | | | | | | | |
| Phage- | N | T | — | L | — | — | — | — | — | W | — | — | H | D |
| 296 | | | | | | | | | | | | | | |
| Phage- | A | H | — | V | — | — | Q | — | — | — | — | — | I | S |
| 297 | | | | | | | | | | | | | | |
| Phage- | N | H | — | E | — | — | N | — | — | — | — | — | I | D |
| 298 | | | | | | | | | | | | | | |
| Phage- | N | T | — | — | — | — | — | — | — | — | — | — | I | D |
| 299 | | | | | | | | | | | | | | |
| Phage- | Y | D | — | V | — | — | — | — | — | W | — | — | H | — |
| 300 | | | | | | | | | | | | | | |
| Phage- | D | S | — | M | — | F | Q | — | — | — | — | — | H | V |
| 301 | | | | | | | | | | | | | | |
| Phage- | — | S | — | M | — | L | N | — | — | — | — | — | I | I |
| 302 | | | | | | | | | | | | | | |
| Phage- | Y | D | — | — | — | F | R | — | — | — | — | — | N | D |
| 303 | | | | | | | | | | | | | | |
| Phage- | Y | S | — | Q | — | I | N | — | — | W | — | — | H | D |
| 304 | | | | | | | | | | | | | | |
| Phage- | N | T | — | L | — | F | Q | — | — | W | — | — | T | N |
| 305 | | | | | | | | | | | | | | |
| Phage- | Y | P | — | M | — | F | Q | — | — | — | — | — | N | D |
| 306 | | | | | | | | | | | | | | |
| Phage- | V | S | — | — | — | F | Q | — | — | — | — | — | T | D |
| 307 | | | | | | | | | | | | | | |
| Phage- | D | — | — | — | — | F | Q | — | — | — | — | — | H | S |
| 308 | | | | | | | | | | | | | | |
| Phage- | Y | N | — | — | — | — | — | — | — | — | — | — | T | D |
| 309 | | | | | | | | | | | | | | |
| Phage- | L | D | — | L | — | — | R | — | I | — | — | — | I | S |
| 310 | | | | | | | | | | | | | | |
| Phage- | N | S | — | — | — | I | N | — | V | — | — | — | I | — |
| 311 | | | | | | | | | | | | | | |
| Phage- | — | D | — | V | — | — | H | — | — | W | — | — | H | D |
| 312 | | | | | | | | | | | | | | |
| Phage- | D | S | — | — | — | — | — | — | — | F | — | — | T | D |
| 313 | | | | | | | | | | | | | | |
| Phage- | D | S | — | V | — | — | — | — | — | W | — | — | I | — |
| 314 | | | | | | | | | | | | | | |
| Phage- | H | N | — | — | — | — | Q | — | — | W | — | — | N | D |
| 315 | | | | | | | | | | | | | | |
| Phage- | F | S | — | — | — | — | R | — | — | — | — | — | N | D |
| 316 | | | | | | | | | | | | | | |
| Phage- | F | S | — | — | — | — | N | — | — | — | — | — | N | D |
| 317 | | | | | | | | | | | | | | |
| Phage | D | H | — | L | — | — | Q | — | — | — | — | — | I | A |
| 318 | | | | | | | | | | | | | | |
| Phage- | H | T | — | — | — | — | — | — | V | — | — | — | L | D |
| 319 | | | | | | | | | | | | | | |
| Phage- | A | D | — | W | — | W | — | — | — | — | — | — | H | D |
| 320 | | | | | | | | | | | | | | |
| Phage- | N | S | — | V | — | W | H | — | — | — | — | — | N | N |
| 321 | | | | | | | | | | | | | | |
| Phage- | — | Y | — | W | — | — | — | — | — | — | — | — | N | D |
| 322 | | | | | | | | | | | | | | |
| Phage- | T | H | — | L | — | F | Q | — | I | — | — | — | Y | D |
| 323 | | | | | | | | | | | | | | |
| Phage- | A | L | — | — | — | I | — | — | — | — | — | — | N | S |
| 324 | | | | | | | | | | | | | | |
| Phage- | — | A | — | — | — | F | Q | — | — | — | — | — | N | A |
| 325 | | | | | | | | | | | | | | |
| Phage- | D | A | — | — | — | — | — | — | R | F | — | — | — | V |
| 326 | | | | | | | | | | | | | | |
| Phage- | H | S | — | — | — | — | — | — | — | — | — | — | N | N |
| 327 | | | | | | | | | | | | | | |
| Phage- | N | H | — | — | — | I | Q | — | — | — | — | — | T | S |
| 328 | | | | | | | | | | | | | | |
| Phage- | T | — | — | — | | L | Q | — | V | — | — | — | N | I |
| 329 | | | | | | | | | | | | | | |
| Phage- | D | S | — | I | — | — | — | — | — | F | — | — | — | D |
| 330 | | | | | | | | | | | | | | |
| Phage- | — | D | — | L | — | — | R | — | — | — | — | — | H | D |
| 331 | | | | | | | | | | | | | | |
| Phage- | — | Y | — | V | — | F | Q | — | — | — | — | — | I | D |
| 332 | | | | | | | | | | | | | | |
| Phage- | Y | H | — | V | — | — | — | — | — | — | — | — | N | V |
| 333 | | | | | | | | | | | | | | |
| Phage- | N | H | — | V | — | — | — | — | V | W | — | Y | I | D |
| 334 | | | | | | | | | | | | | | |
| Phage- | T | D | — | L | — | W | R | — | — | — | — | — | Y | D |
| 335 | | | | | | | | | | | | | | |
| Phage- | H | Y | — | E | — | L | Q | — | V | S | — | — | I | — |
| 336 | | | | | | | | | | | | | | |
| Phage- | F | S | — | M | — | E | — | — | — | W | — | — | L | V |
| 337 | | | | | | | | | | | | | | |
| Phage- | T | S | — | M | — | — | — | — | I | F | — | — | N | — |
| 338 | | | | | | | | | | | | | | |
| Phage- | N | — | — | Y | — | — | Q | — | V | — | — | — | I | — |
| 339 | | | | | | | | | | | | | | |
| Phage- | A | N | — | V | — | — | H | — | — | W | — | — | I | D |
| 340 | | | | | | | | | | | | | | |
| Phage- | Y | D | — | — | — | F | — | — | — | — | — | — | H | V |
| 341 | | | | | | | | | | | | | | |
| Phage- | A | H | — | V | — | — | R | — | — | — | — | — | I | N |
| 342 | | | | | | | | | | | | | | |
| Phage- | N | T | — | — | — | | N | — | — | W | — | — | I | D |
| 343 | | | | | | | | | | | | | | |
| Phage- | Y | T | — | — | — | — | Q | — | — | — | — | — | T | V |
| 344 | | | | | | | | | | | | | | |
| Phage- | N | H | — | V | — | F | Q | — | — | — | — | — | H | D |
| 345 | | | | | | | | | | | | | | |
| Phage- | N | A | — | — | — | — | N | — | — | W | — | — | T | V |
| 346 | | | | | | | | | | | | | | |
| Phage- | N | S | — | L | — | F | H | — | — | — | — | — | — | N |
| 347 | | | | | | | | | | | | | | |
| Phage- | — | S | — | — | — | — | — | — | — | — | — | — | T | N |
| 348 | | | | | | | | | | | | | | |
| Phage- | N | T | — | — | — | F | H | — | V | — | — | — | N | D |
| 349 | | | | | | | | | | | | | | |
| Phage- | — | D | — | — | — | I | Q | — | V | — | — | — | H | D |
| 350 | | | | | | | | | | | | | | |
| Phage- | N | L | — | — | — | — | — | — | — | — | — | — | I | D |
| 351 | | | | | | | | | | | | | | |
| Phage- | H | D | — | — | — | F | Q | — | V | — | — | — | T | V |
| 352 | | | | | | | | | | | | | | |
| Phage- | V | N | — | M | — | — | R | — | — | — | — | — | I | D |
| 353 | | | | | | | | | | | | | | |
| Phage- | Y | S | — | Y | — | — | — | — | — | — | — | — | T | H |
| 354 | | | | | | | | | | | | | | |
| Phage- | V | S | — | L | — | — | — | — | — | — | — | — | H | S |
| 355 | | | | | | | | | | | | | | |
| Phage- | V | D | — | M | — | I | — | — | — | W | — | — | H | N |
| 356 | | | | | | | | | | | | | | |
| Phage- | D | T | — | V | — | — | — | — | — | W | — | — | I | D |
| 357 | | | | | | | | | | | | | | |
| Phage- | N | T | — | V | — | — | — | — | — | W | — | — | I | N |
| 358 | | | | | | | | | | | | | | |
| Phage- | V | S | — | — | — | — | — | — | — | — | — | — | H | S |
| 359 | | | | | | | | | | | | | | |
| Phage- | P | S | — | — | — | — | N | — | — | F | — | — | I | D |
| 360 | | | | | | | | | | | | | | |
| Phage- | N | S | — | S | — | — | R | — | — | — | — | — | I | V |
| 361 | | | | | | | | | | | | | | |
| Phage- | T | S | — | — | — | — | Q | — | — | F | — | — | H | A |
| 362 | | | | | | | | | | | | | | |
| Phage- | — | D | — | — | — | — | — | — | — | — | — | — | N | — |
| 363 | | | | | | | | | | | | | | |
| Phage- | H | S | — | — | — | — | — | — | — | — | — | — | T | N |
| 364 | | | | | | | | | | | | | | |
| Phage- | A | T | — | M | — | — | N | — | — | W | — | — | I | N |
| 365 | | | | | | | | | | | | | | |
| Phage- | A | S | — | V | — | F | Q | — | — | — | — | — | H | S |
| 366 | | | | | | | | | | | | | | |
| Phage- | N | H | — | V | — | — | N | — | — | — | — | — | I | N |
| 367 | | | | | | | | | | | | | | |
| Phage- | N | N | — | — | — | H | — | — | — | F | — | — | — | N |
| 368 | | | | | | | | | | | | | | |
| Phage- | A | A | — | — | — | — | — | — | — | — | — | — | — | D |
| 369 | | | | | | | | | | | | | | |
| Phage- | — | D | — | I | — | — | R | — | — | — | — | — | I | D |
| 370 | | | | | | | | | | | | | | |
| Phage- | A | T | — | — | — | — | Q | — | — | — | — | — | H | S |
| 371 | | | | | | | | | | | | | | |
| Phage- | — | T | — | V | — | — | R | — | — | — | — | — | N | A |
| 372 | | | | | | | | | | | | | | |
| Phage- | T | D | — | M | — | — | R | — | — | — | — | — | N | N |
| 373 | | | | | | | | | | | | | | |
| Phage- | Y | T | — | — | — | — | N | — | — | — | — | — | T | H |
| 374 | | | | | | | | | | | | | | |
| Phage- | Y | S | — | I | — | — | R | — | — | — | — | — | A | D |
| 375 | | | | | | | | | | | | | | |
| Phage- | N | T | — | V | — | — | N | — | — | — | — | — | I | Y |
| 376 | | | | | | | | | | | | | | |
| Phage- | T | T | — | — | — | F | Q | — | — | — | — | — | H | V |
| 377 | | | | | | | | | | | | | | |
| Phage- | N | I | — | M | — | — | R | — | — | — | — | — | I | H |
| 378 | | | | | | | | | | | | | | |
| Phage- | F | S | — | V | — | — | — | — | V | — | — | — | H | I |
| 379 | | | | | | | | | | | | | | |
| Phage- | N | — | — | I | — | — | Q | — | — | — | — | — | I | D |
| 380 | | | | | | | | | | | | | | |
| Phage- | D | T | — | S | — | — | R | — | V | F | — | — | I | V |
| 381 | | | | | | | | | | | | | | |
| Phage- | D | L | — | — | — | I | R | — | — | — | — | — | I | N |
| 382 | | | | | | | | | | | | | | |
| Phage- | A | H | — | V | — | — | N | — | — | — | — | — | T | D |
| 383 | | | | | | | | | | | | | | |
| Phage- | D | N | — | I | — | — | — | — | — | Q | — | — | T | I |
| 384 | | | | | | | | | | | | | | |
| Phage- | L | T | — | — | — | — | — | — | — | — | — | — | H | A |
| 385 | | | | | | | | | | | | | | |
| Phage- | N | T | — | L | — | — | D | — | — | W | — | — | I | D |
| 386 | | | | | | | | | | | | | | |
| Phage- | P | A | — | — | — | — | — | — | I | — | — | — | I | D |
| 387 | | | | | | | | | | | | | | |
| Phage- | N | T | — | — | — | F | R | — | — | W | — | — | N | D |
| 388 | | | | | | | | | | | | | | |
| Phage- | H | S | — | — | — | — | M | — | — | — | — | — | I | — |
| 389 | | | | | | | | | | | | | | |
| Phage- | P | T | — | M | — | — | R | — | — | — | — | — | — | — |
| 390 | | | | | | | | | | | | | | |
| Phage- | N | Y | — | — | — | L | — | — | — | — | — | — | I | D |
| 391 | | | | | | | | | | | | | | |
| Phage- | Y | S | — | M | — | — | — | — | — | — | — | — | H | N |
| 392 | | | | | | | | | | | | | | |
| Phage- | N | I | — | L | — | L | — | — | I | — | — | — | H | V |
| 393 | | | | | | | | | | | | | | |
| Phage- | Y | T | — | L | — | — | N | — | — | — | — | — | T | V |
| 394 | | | | | | | | | | | | | | |
| Phage- | T | H | — | L | — | F | Q | — | — | — | — | — | N | V |
| 395 | | | | | | | | | | | | | | |
| Phage- | — | T | — | L | — | F | Q | — | — | W | — | — | H | D |
| 396 | | | | | | | | | | | | | | |
| Phage- | T | H | — | — | — | — | Q | — | I | — | — | — | N | I |
| 397 | | | | | | | | | | | | | | |
| Phage- | N | S | — | M | — | — | R | — | V | L | — | — | I | — |
| 398 | | | | | | | | | | | | | | |
| Phage- | D | T | — | — | — | — | R | — | — | — | — | — | I | Y |
| 399 | | | | | | | | | | | | | | |
| Phage- | — | D | — | W | — | — | M | — | — | — | — | — | H | V |
| 400 | | | | | | | | | | | | | | |
| Phage- | N | S | — | V | — | F | — | — | — | — | — | — | I | N |
| 401 | | | | | | | | | | | | | | |
| Phage- | N | S | — | M | — | — | — | — | F | — | — | — | I | D |
| 402 | | | | | | | | | | | | | | |
| Phage- | N | S | — | V | — | — | — | — | — | — | — | — | I | N |
| 403 | | | | | | | | | | | | | | |
| Phage- | N | A | — | — | — | — | Q | — | — | — | — | — | — | D |
| 404 | | | | | | | | | | | | | | |
| Phage- | H | T | — | — | — | M | E | — | — | W | — | — | N | D |
| 405 | | | | | | | | | | | | | | |
| Phage- | N | Y | — | — | — | — | — | — | — | — | — | — | I | — |
| 406 | | | | | | | | | | | | | | |
| Phage- | N | S | — | W | — | — | — | — | — | — | — | — | T | D |
| 407 | | | | | | | | | | | | | | |
| Phage- | N | — | — | M | — | F | R | — | I | — | — | — | N | Y |
| 408 | | | | | | | | | | | | | | |
| Phage- | N | D | — | — | — | F | R | — | I | — | — | — | N | D |
| 409 | | | | | | | | | | | | | | |
| Phage- | Y | S | — | M | — | — | R | — | — | — | — | — | N | L |
| 410 | | | | | | | | | | | | | | |
| Phage- | N | T | — | — | — | — | R | — | — | — | — | — | — | N |
| 411 | | | | | | | | | | | | | | |
| Phage- | D | H | — | L | — | — | — | — | — | F | — | — | T | I |
| 412 | | | | | | | | | | | | | | |
| Phage- | Y | D | — | L | — | — | Q | — | I | — | — | — | H | V |
| 413 | | | | | | | | | | | | | | |
| Phage- | D | S | — | — | — | F | Q | — | — | W | — | — | T | D |
| 414 | | | | | | | | | | | | | | |
| Phage- | F | T | — | — | — | — | N | — | — | — | — | — | N | S |
| 415 | | | | | | | | | | | | | | |
| Phage- | A | A | — | — | — | — | — | — | — | — | — | — | T | D |
| 416 | | | | | | | | | | | | | | |
| Phage- | Y | S | — | — | — | — | — | — | I | — | — | — | — | V |
| 417 | | | | | | | | | | | | | | |
| Phage- | N | H | — | L | — | — | — | — | — | — | — | — | I | N |
| 418 | | | | | | | | | | | | | | |
| Phage- | F | S | — | M | — | — | Q | — | — | — | — | — | H | D |
| 419 | | | | | | | | | | | | | | |
| Phage- | A | S | — | Y | — | — | M | — | I | — | — | — | T | I |
| 420 | | | | | | | | | | | | | | |
| Phage- | Q | D | — | W | — | — | D | — | — | W | — | — | I | D |
| 421 | | | | | | | | | | | | | | |
| Phage- | A | D | — | I | — | — | R | — | — | — | — | — | I | D |
| 422 | | | | | | | | | | | | | | |
| Phage- | — | D | — | I | — | — | R | — | — | W | — | — | N | — |
| 423 | | | | | | | | | | | | | | |
| Phage- | N | S | — | — | — | F | R | — | — | — | — | — | T | — |
| 424 | | | | | | | | | | | | | | |
| Phage- | — | S | — | — | — | F | Q | — | — | W | — | — | N | D |
| 425 | | | | | | | | | | | | | | |
| Phage- | F | T | — | — | — | — | Q | — | V | — | — | — | H | S |
| 426 | | | | | | | | | | | | | | |
| Phage- | F | S | — | Y | — | — | R | — | — | — | — | — | N | D |
| 427 | | | | | | | | | | | | | | |
| Phage- | H | D | — | — | — | — | R | — | — | — | — | — | N | D |
| 428 | | | | | | | | | | | | | | |
| Phage- | N | Y | — | V | — | L | N | — | I | — | — | — | I | A |
| 429 | | | | | | | | | | | | | | |
| Phage- | D | T | — | — | — | L | — | — | — | W | — | — | I | D |
| 430 | | | | | | | | | | | | | | |
| Phage- | T | S | — | — | — | — | H | — | I | — | — | — | H | S |
| 431 | | | | | | | | | | | | | | |
| Phage- | Y | S | — | — | — | — | R | — | — | — | — | — | N | V |
| 432 | | | | | | | | | | | | | | |
| Phage- | T | D | — | L | — | — | R | — | — | W | — | — | H | I |
| 433 | | | | | | | | | | | | | | |
| Phage- | L | A | — | — | — | — | — | — | — | — | — | — | N | D |
| 434 | | | | | | | | | | | | | | |
| Phage- | N | A | — | — | — | — | — | — | V | — | — | — | T | Y |
| 435 | | | | | | | | | | | | | | |
| Phage- | L | S | — | M | — | — | H | — | — | — | — | — | H | I |
| 436 | | | | | | | | | | | | | | |
| Phage- | F | T | M | — | — | — | Q | — | V | F | — | — | H | A |
| 437 | | | | | | | | | | | | | | |
| Phage- | N | S | — | — | — | — | Q | — | I | W | — | — | T | D |
| 438 | | | | | | | | | | | | | | |
| Phage- | H | A | — | I | — | — | N | — | — | W | — | — | N | — |
| 439 | | | | | | | | | | | | | | |
| Phage- | N | H | — | M | — | L | N | — | — | W | — | — | I | D |
| 440 | | | | | | | | | | | | | | |
| Phage- | N | Y | — | W | — | — | H | — | — | — | — | — | T | D |
| 441 | | | | | | | | | | | | | | |
| Phage- | N | A | — | M | — | — | N | — | I | W | — | — | I | D |
| 442 | | | | | | | | | | | | | | |
| Phage- | N | T | — | — | — | W | Q | — | — | — | — | — | I | V |
| 443 | | | | | | | | | | | | | | |
| Phage- | N | H | — | Y | — | W | — | — | — | — | — | — | I | D |
| 444 | | | | | | | | | | | | | | |
| Phage- | N | L | — | — | — | — | R | — | — | — | — | — | I | D |
| 445 | | | | | | | | | | | | | | |
| Phage- | Y | S | — | M | — | — | N | — | — | W | — | — | N | — |
| 446 | | | | | | | | | | | | | | |
| Phage- | G | S | — | L | — | — | — | — | — | — | — | — | I | D |
| 447 | | | | | | | | | | | | | | |
| Phage- | L | D | — | V | — | — | R | — | — | — | — | — | I | N |
| 448 | | | | | | | | | | | | | | |
| Phage- | N | A | — | L | — | — | Q | — | V | — | — | — | I | D |
| 449 | | | | | | | | | | | | | | |
| Phage- | Y | T | — | — | — | L | Q | — | V | — | — | Y | H | I |
| 450 | | | | | | | | | | | | | | |
| Phage- | N | Y | — | V | — | — | — | — | — | E | — | — | I | V |
| 451 | | | | | | | | | | | | | | |
| Phage- | — | F | — | T | — | I | Q | — | I | — | — | — | — | S |
| 452 | | | | | | | | | | | | | | |
| Phage- | L | A | — | — | — | Y | Q | — | — | F | — | — | I | N |
| 453 | | | | | | | | | | | | | | |
| Phage- | A | H | — | M | — | — | — | — | — | — | — | — | I | V |
| 454 | | | | | | | | | | | | | | |
| Phage- | — | D | — | I | — | — | — | — | — | — | — | — | Y | D |
| 455 | | | | | | | | | | | | | | |
| Phage- | H | A | — | — | — | — | N | — | — | W | — | — | N | — |
| 456 | | | | | | | | | | | | | | |
| Phage- | Y | T | — | — | — | I | Q | — | V | H | — | — | T | V |
| 457 | | | | | | | | | | | | | | |
| Phage- | D | N | — | R | — | — | R | — | — | — | — | — | I | D |
| 458 | | | | | | | | | | | | | | |
| Phage- | D | F | — | R | — | T | — | — | — | F | — | — | I | D |
| 459 | | | | | | | | | | | | | | |
| Phage- | D | H | — | L | — | F | R | — | V | — | — | — | H | I |
| 460 | | | | | | | | | | | | | | |
| Phage- | Y | — | — | L | — | — | — | — | V | — | — | — | N | E |
| 461 | | | | | | | | | | | | | | |
| Phage- | F | S | — | M | — | I | — | — | — | — | — | — | T | — |
| 462 | | | | | | | | | | | | | | |
| Phage- | T | H | — | L | — | — | — | — | I | — | — | — | H | V |
| 463 | | | | | | | | | | | | | | |
| Phage- | A | S | — | — | — | L | Q | — | — | — | — | — | N | Y |
| 464 | | | | | | | | | | | | | | |
| Phage- | H | D | — | V | — | — | N | — | — | W | — | — | N | D |
| 465 | | | | | | | | | | | | | | |
| Phage- | A | A | — | E | — | F | Q | — | — | W | — | — | — | I |
| 466 | | | | | | | | | | | | | | |
| Phage- | L | D | — | I | — | F | Q | — | V | — | — | — | Y | A |
| 467 | | | | | | | | | | | | | | |
| Phage- | N | D | — | H | — | — | M | — | — | — | — | — | — | Y |
| 468 | | | | | | | | | | | | | | |
| Phage- | N | T | — | L | — | I | E | — | — | — | — | — | I | D |
| 469 | | | | | | | | | | | | | | |
| Phage- | A | S | — | — | — | I | Q | — | — | — | — | — | T | D |
| 470 | | | | | | | | | | | | | | |
| Phage- | N | Y | — | V | — | — | N | — | — | W | — | — | I | S |
| 471 | | | | | | | | | | | | | | |
| Phage- | N | — | — | — | — | — | — | — | Y | — | — | — | I | I |
| 472 | | | | | | | | | | | | | | |
| Phage- | N | Y | — | — | L | Q | — | V | — | — | — | — | — | D |
| 473 | | | | | | | | | | | | | | |
| Phage- | F | S | — | H | — | F | — | — | V | — | — | — | — | V |
| 474 | | | | | | | | | | | | | | |
| Phage- | A | S | — | — | — | I | — | — | — | W | — | — | N | N |
| 475 | | | | | | | | | | | | | | |
| Phage- | Y | S | — | — | — | — | — | — | — | — | — | — | N | A |
| 476 | | | | | | | | | | | | | | |
| Phage- | T | Y | — | Y | — | F | Q | — | — | — | — | — | H | V |
| 477 | | | | | | | | | | | | | | |
| Phage- | N | — | — | — | — | I | N | — | — | W | — | — | T | — |
| 478 | | | | | | | | | | | | | | |
| Phage- | N | D | — | — | — | I | M | — | V | — | — | — | N | — |
| 479 | | | | | | | | | | | | | | |
| Phage- | N | T | — | — | — | — | R | — | — | — | — | — | N | S |
| 480 | | | | | | | | | | | | | | |
| Phage- | F | D | — | — | — | — | R | — | — | — | — | — | T | N |
| 481 | | | | | | | | | | | | | | |
| Phage- | N | F | — | W | — | — | N | — | V | — | — | — | I | D |
| 482 | | | | | | | | | | | | | | |
| Phage- | T | Y | — | — | — | F | Q | — | — | — | — | — | N | L |
| 483 | | | | | | | | | | | | | | |
| Phage- | N | S | — | M | — | — | M | — | — | — | — | — | I | — |
| 484 | | | | | | | | | | | | | | |
| Phage- | A | S | — | — | — | — | M | — | — | — | — | — | N | V |
| 485 | | | | | | | | | | | | | | |
| Phage- | N | T | — | — | — | — | — | — | — | — | — | — | I | A |
| 486 | | | | | | | | | | | | | | |
| Phage- | H | S | — | — | — | I | E | — | — | — | — | — | N | D |
| 487 | | | | | | | | | | | | | | |
| Phage- | A | D | — | I | — | — | M | — | V | — | — | — | — | S |
| 488 | | | | | | | | | | | | | | |
| Phage- | H | A | — | M | — | — | R | — | — | — | — | — | N | A |
| 489 | | | | | | | | | | | | | | |
| Phage- | Y | P | — | V | — | F | — | — | — | — | — | — | N | H |
| 490 | | | | | | | | | | | | | | |
| Phage- | — | H | — | — | — | Y | — | — | — | — | — | — | N | Y |
| 491 | | | | | | | | | | | | | | |
| Phage- | — | D | — | W | — | — | — | — | — | — | — | — | H | V |
| 492 | | | | | | | | | | | | | | |
| Phage- | F | S | — | — | — | — | — | — | — | — | — | — | H | A |
| 493 | | | | | | | | | | | | | | |
| Phage- | H | T | — | M | — | F | Q | — | — | — | — | — | H | N |
| 494 | | | | | | | | | | | | | | |
| Phage- | Y | S | — | — | — | — | N | — | — | W | — | — | — | D |
| 495 | | | | | | | | | | | | | | |
| Phage- | Y | P | — | — | — | — | R | — | — | — | — | — | N | D |
| 496 | | | | | | | | | | | | | | |
| Phage- | N | T | — | L | — | F | Q | — | — | — | — | — | I | D |
| 497 | | | | | | | | | | | | | | |
| Phage- | N | S | — | L | — | I | H | — | — | W | — | — | I | D |
| 498 | | | | | | | | | | | | | | |
| Phage- | T | N | — | W | — | — | R | — | V | L | — | — | H | S |
| 499 | | | | | | | | | | | | | | |
| Phage- | H | A | — | — | — | — | — | — | — | — | — | — | N | S |
| 500 | | | | | | | | | | | | | | |
| Phage- | H | T | — | V | — | — | R | — | — | — | — | — | H | N |
| 501 | | | | | | | | | | | | | | |
| Phage- | N | H | — | V | — | F | — | — | — | — | — | — | — | S |
| 502 | | | | | | | | | | | | | | |
| Phage- | A | T | — | L | — | — | — | — | — | W | — | — | H | — |
| 503 | | | | | | | | | | | | | | |
| Phage- | D | H | — | L | — | — | — | — | — | F | — | — | I | A |
| 504 | | | | | | | | | | | | | | |
| Phage- | L | D | — | — | — | W | R | — | — | — | — | — | N | A |
| 505 | | | | | | | | | | | | | | |
| Phage- | F | D | — | — | — | — | R | — | — | — | — | R | N | D |
| 506 | | | | | | | | | | | | | | |
| Phage- | Y | S | — | — | — | F | H | — | — | — | — | — | I | D |
| 507 | | | | | | | | | | | | | | |
| Phage- | N | T | — | M | — | — | Q | — | V | — | — | — | I | I |
| 508 | | | | | | | | | | | | | | |
| Phage- | N | A | — | — | — | — | R | — | — | — | — | — | N | F |
| 509 | | | | | | | | | | | | | | |
| Phage- | — | S | — | M | — | — | R | — | — | — | — | — | I | I |
| 510 | | | | | | | | | | | | | | |
| Phage- | H | — | — | — | — | — | Q | — | I | — | — | — | T | V |
| 511 | | | | | | | | | | | | | | |
| Phage- | — | S | — | M | — | — | N | — | — | — | — | — | T | V |
| 512 | | | | | | | | | | | | | | |
| Phage- | H | S | — | W | — | F | Q | — | — | — | — | — | N | A |
| 513 | | | | | | | | | | | | | | |
| Phage- | N | A | — | Y | — | — | — | — | — | W | — | — | I | D |
| 514 | | | | | | | | | | | | | | |
| Phage- | T | S | M | L | — | F | Q | — | — | — | — | — | H | V |
| 515 | | | | | | | | | | | | | | |
| Phage- | Y | D | — | I | — | — | — | — | — | W | — | — | T | — |
| 516 | | | | | | | | | | | | | | |
| Phage- | A | D | — | — | — | — | — | — | — | — | — | — | H | V |
| 517 | | | | | | | | | | | | | | |
| Phage- | A | S | — | — | — | — | — | — | — | — | — | — | N | A |
| 518 | | | | | | | | | | | | | | |
| Phage- | H | S | — | — | — | — | R | — | — | — | — | — | N | D |
| 519 | | | | | | | | | | | | | | |
| Phage- | N | Y | — | V | — | — | — | — | — | W | — | Y | I | N |
| 520 | | | | | | | | | | | | | | |
| Phage- | N | D | — | L | — | F | Q | — | I | — | — | — | N | V |
| 521 | | | | | | | | | | | | | | |
| Phage- | T | D | — | — | — | Y | R | — | — | W | — | — | H | D |
| 522 | | | | | | | | | | | | | | |
| Phage- | I | A | — | L | — | F | Q | — | V | — | — | — | H | I |
| 523 | | | | | | | | | | | | | | |
| Phage- | I | D | — | V | — | F | R | — | I | W | — | — | N | — |
| 524 | | | | | | | | | | | | | | |
| Phage- | N | A | — | — | — | F | H | — | — | — | — | — | I | D |
| 525 | | | | | | | | | | | | | | |
| Phage- | N | N | — | — | — | — | R | — | — | — | — | — | N | I |
| 526 | | | | | | | | | | | | | | |
| Phage- | D | T | — | — | — | — | Q | — | I | W | — | — | T | A |
| 527 | | | | | | | | | | | | | | |
| Phage- | Y | H | — | — | — | — | R | — | — | — | — | — | — | S |
| 528 | | | | | | | | | | | | | | |
| Phage- | F | S | — | L | — | — | R | — | — | — | — | — | T | V |
| 529 | | | | | | | | | | | | | | |
| Phage- | D | T | — | V | — | — | Q | — | — | — | — | — | I | S |
| 530 | | | | | | | | | | | | | | |
| Phage- | D | H | — | V | — | — | — | — | — | L | — | — | I | N |
| 531 | | | | | | | | | | | | | | |
| Phage- | N | Y | — | — | — | — | — | — | — | — | — | — | I | D |
| 532 | | | | | | | | | | | | | | |
| Phage- | H | S | — | — | — | — | N | — | — | — | — | — | T | S |
| 533 | | | | | | | | | | | | | | |
| Phage- | N | F | — | M | — | — | — | — | — | S | — | — | I | D |
| 534 | | | | | | | | | | | | | | |
| Phage- | F | A | — | — | — | — | N | — | — | — | — | — | N | D |
| 535 | | | | | | | | | | | | | | |
| Phage- | — | Y | — | Y | — | L | R | — | I | — | — | — | N | — |
| 536 | | | | | | | | | | | | | | |
| Phage- | T | S | — | L | — | — | — | — | — | W | — | — | N | V |
| 537 | | | | | | | | | | | | | | |
| Phage- | F | S | — | M | — | — | R | — | — | — | — | — | H | — |
| 538 | | | | | | | | | | | | | | |
| Phage- | N | Y | — | Y | — | L | — | — | — | — | — | — | N | I |
| 539 | | | | | | | | | | | | | | |
| Phage- | N | H | — | L | — | — | R | — | — | — | — | — | I | — |
| 540 | | | | | | | | | | | | | | |
| Phage- | N | Y | — | — | — | — | Q | — | V | — | — | — | I | A |
| 541 | | | | | | | | | | | | | | |
| Phage- | N | T | — | M | — | — | — | — | — | F | — | — | I | D |
| 542 | | | | | | | | | | | | | | |
| Phage- | D | Y | — | L | — | F | Q | — | V | — | — | — | H | I |
| 543 | | | | | | | | | | | | | | |
| Phage- | H | A | — | — | — | — | R | — | — | — | — | — | — | D |
| 544 | | | | | | | | | | | | | | |
| Phage- | — | D | — | — | — | — | M | — | V | — | — | — | H | I |
| 545 | | | | | | | | | | | | | | |
| Phage- | N | — | — | — | — | — | — | — | — | — | — | — | I | D |
| 546 | | | | | | | | | | | | | | |
| Phage- | — | S | — | — | — | F | Q | — | — | — | — | — | — | D |
| 547 | | | | | | | | | | | | | | |
| Phage- | N | T | — | — | — | — | — | — | — | — | — | — | T | S |
| 548 | | | | | | | | | | | | | | |
| Phage- | P | H | — | M | — | — | Q | — | I | — | — | — | I | I |
| 549 | | | | | | | | | | | | | | |
| Phage- | D | L | — | V | — | L | R | — | W | F | — | — | N | D |
| 550 | | | | | | | | | | | | | | |
| Phage- | H | D | — | — | — | — | R | — | — | W | — | — | N | A |
| 551 | | | | | | | | | | | | | | |
| Phage- | N | L | — | M | — | — | Q | — | — | — | — | — | I | A |
| 552 | | | | | | | | | | | | | | |
| Phage- | D | T | — | W | — | — | R | — | — | — | — | — | I | N |
| 553 | | | | | | | | | | | | | | |
| Phage- | N | — | — | V | — | — | N | — | — | W | — | — | I | D |
| 554 | | | | | | | | | | | | | | |
| Phage- | — | Y | — | — | — | F | — | — | — | — | — | — | N | D |
| 555 | | | | | | | | | | | | | | |
| Phage- | N | N | — | W | — | F | Q | — | — | — | — | — | H | V |
| 556 | | | | | | | | | | | | | | |
| Phage- | L | Y | — | M | — | — | — | — | — | — | — | — | I | N |
| 557 | | | | | | | | | | | | | | |
| Phage- | N | D | — | I | — | — | R | — | — | — | — | — | H | Y |
| 558 | | | | | | | | | | | | | | |
| Phage- | N | H | — | — | — | — | R | — | — | — | — | — | T | — |
| 559 | | | | | | | | | | | | | | |
| Phage- | N | A | — | L | — | — | — | — | — | — | — | — | I | D |
| 560 | | | | | | | | | | | | | | |
| Phage- | L | A | — | — | — | — | — | — | — | — | — | — | H | D |
| 561 | | | | | | | | | | | | | | |
| Phage- | N | A | — | V | — | — | Y | — | I | — | — | — | H | V |
| 562 | | | | | | | | | | | | | | |
| Phage- | N | N | — | Y | — | — | — | — | — | W | — | — | — | A |
| 563 | | | | | | | | | | | | | | |
| Phage- | Y | S | — | M | — | — | — | — | — | — | — | — | H | A |
| 564 | | | | | | | | | | | | | | |
| Phage- | N | A | — | L | — | — | N | — | — | W | — | — | I | D |
| 565 | | | | | | | | | | | | | | |
| Phage- | D | T | — | L | — | — | H | — | — | F | — | — | I | D |
| 566 | | | | | | | | | | | | | | |
| Phage- | N | N | — | Y | — | F | Q | — | V | F | — | — | I | A |
| 567 | | | | | | | | | | | | | | |
| Phage- | A | S | — | M | — | — | — | — | — | — | — | — | H | A |
| 568 | | | | | | | | | | | | | | |
| Phage- | T | A | — | — | — | F | Q | — | V | — | — | — | H | D |
| 569 | | | | | | | | | | | | | | |
| Phage- | N | — | — | V | — | — | Q | — | I | — | — | — | — | S |
| 570 | | | | | | | | | | | | | | |
| Phage- | N | N | — | M | — | F | — | — | — | — | — | — | H | I |
| 571 | | | | | | | | | | | | | | |
| Phage- | — | S | — | L | — | — | N | — | — | W | — | — | T | — |
| 572 | | | | | | | | | | | | | | |
| Phage- | N | S | — | — | — | — | M | — | I | — | — | — | H | D |
| 573 | | | | | | | | | | | | | | |
| Phage- | N | H | — | — | — | I | — | — | — | — | — | — | H | A |
| 574 | | | | | | | | | | | | | | |
| Phage- | F | S | — | — | — | — | — | — | — | — | — | — | T | N |
| 575 | | | | | | | | | | | | | | |
| Phage- | N | T | — | — | — | — | Q | — | V | F | — | — | T | Y |
| 576 | | | | | | | | | | | | | | |
| Phage- | N | A | — | — | — | F | — | — | — | — | — | — | T | Y |
| 577 | | | | | | | | | | | | | | |
| Phage- | N | T | — | L | — | — | — | — | — | — | — | — | I | D |
| 578 | | | | | | | | | | | | | | |
| Phage- | — | H | — | — | — | F | Q | — | — | — | — | — | N | A |
| 579 | | | | | | | | | | | | | | |
| Phage- | Y | T | — | — | — | F | Q | — | — | — | — | — | N | V |
| 580 | | | | | | | | | | | | | | |
| Phage- | H | S | — | — | — | — | — | — | — | — | — | — | T | I |
| 581 | | | | | | | | | | | | | | |
| Phage- | D | Y | — | L | — | F | R | — | — | — | — | — | Y | D |
| 582 | | | | | | | | | | | | | | |
| Phage- | Y | N | — | — | — | — | R | — | — | — | — | — | — | A |
| 583 | | | | | | | | | | | | | | |
| Phage- | Y | S | — | V | — | — | — | — | — | W | — | — | N | I |
| 584 | | | | | | | | | | | | | | |
| Phage- | F | N | — | — | — | — | — | — | — | — | — | — | N | D |
| 585 | | | | | | | | | | | | | | |
| Phage- | V | A | — | — | — | — | — | — | I | — | — | — | N | F |
| 586 | | | | | | | | | | | | | | |
| Phage- | D | Y | — | L | — | — | H | — | — | F | — | — | I | V |
| 587 | | | | | | | | | | | | | | |
| Phage- | D | T | — | L | — | — | — | — | — | F | — | — | I | D |
| 588 | | | | | | | | | | | | | | |
| Phage- | D | — | — | V | — | — | N | — | — | F | — | — | I | D |
| 589 | | | | | | | | | | | | | | |
| Phage- | N | Y | — | Y | — | — | Q | — | V | — | — | — | I | I |
| 590 | | | | | | | | | | | | | | |
| Phage- | N | S | — | Q | — | — | R | — | I | — | — | — | I | N |
| 591 | | | | | | | | | | | | | | |
| Phage- | Y | S | — | — | — | — | Q | — | — | — | — | — | N | A |
| 592 | | | | | | | | | | | | | | |
| Phage- | F | S | — | — | — | — | — | — | — | — | — | — | N | — |
| 593 | | | | | | | | | | | | | | |
| Phage- | N | N | — | V | — | — | R | — | — | — | — | — | I | A |
| 594 | | | | | | | | | | | | | | |
| Phage- | N | S | — | L | — | — | — | — | I | — | — | — | I | D |
| 595 | | | | | | | | | | | | | | |
| Phage- | N | F | — | V | — | — | R | — | — | — | — | — | I | D |
| 596 | | | | | | | | | | | | | | |
| Phage- | H | — | — | — | — | — | R | — | — | — | — | — | T | N |
| 597 | | | | | | | | | | | | | | |
| Phage- | — | I | — | L | — | Y | Q | — | — | L | — | — | I | D |
| 598 | | | | | | | | | | | | | | |
| Phage- | N | F | — | — | — | — | R | — | — | — | — | — | I | D |
| 599 | | | | | | | | | | | | | | |
| Phage- | D | T | — | V | — | — | — | — | — | L | — | — | T | F |
| 600 | | | | | | | | | | | | | | |
| Phage- | Y | S | — | L | — | — | — | — | V | — | — | — | H | I |
| 601 | | | | | | | | | | | | | | |
| Phage- | D | T | — | I | — | — | R | — | — | — | — | — | I | N |
| 602 | | | | | | | | | | | | | | |
| Phage- | A | H | — | L | — | — | — | — | — | W | — | — | S | V |
| 603 | | | | | | | | | | | | | | |
| Phage- | F | S | — | — | — | — | — | — | V | — | — | — | — | S |
| 604 | | | | | | | | | | | | | | |
| Phage- | D | T | — | — | — | — | R | — | — | W | — | — | N | A |
| 605 | | | | | | | | | | | | | | |
| Phage- | D | T | — | V | — | — | R | — | — | F | — | — | P | I |
| 606 | | | | | | | | | | | | | | |
| Phage- | — | D | — | — | — | — | — | — | — | W | — | — | H | D |
| 607 | | | | | | | | | | | | | | |
| Phage- | F | H | — | L | — | — | Q | — | — | — | — | — | H | D |
| 608 | | | | | | | | | | | | | | |
| Phage- | N | S | — | — | — | — | — | — | V | F | — | — | I | V |
| 609 | | | | | | | | | | | | | | |
| Phage- | D | S | — | V | — | — | — | — | — | F | — | — | — | V |
| 610 | | | | | | | | | | | | | | |
| Phage- | Y | — | — | — | — | — | — | — | I | — | — | — | H | V |
| 611 | | | | | | | | | | | | | | |
| Phage- | — | S | — | W | — | — | — | — | — | W | — | Y | H | V |
| 612 | | | | | | | | | | | | | | |
| Phage- | N | — | — | — | — | — | Q | — | I | — | — | — | N | D |
| 613 | | | | | | | | | | | | | | |
| Phage- | Y | S | — | — | — | — | Q | — | — | W | — | — | H | D |
| 614 | | | | | | | | | | | | | | |
| Phage- | F | D | — | — | — | — | R | — | — | — | — | — | N | Y |
| 615 | | | | | | | | | | | | | | |
| Phage- | P | A | — | K | — | — | — | — | — | F | — | — | I | D |
| 616 | | | | | | | | | | | | | | |
| Phage- | F | S | — | — | — | — | — | — | — | — | — | — | N | D |
| 617 | | | | | | | | | | | | | | |
| Phage- | A | S | — | L | — | — | — | — | — | W | — | — | H | V |
| 618 | | | | | | | | | | | | | | |
| Phage- | N | S | — | W | — | — | N | — | — | F | — | — | T | D |
| 619 | | | | | | | | | | | | | | |
| Phage- | T | Y | — | L | — | F | Q | — | — | — | — | — | N | V |
| 620 | | | | | | | | | | | | | | |
| Phage- | F | S | — | — | — | — | Q | — | — | — | — | — | T | — |
| 621 | | | | | | | | | | | | | | |
| Phage- | Y | A | — | — | — | — | Q | — | — | — | — | — | N | — |
| 622 | | | | | | | | | | | | | | |
| Phage- | N | Y | — | V | — | — | N | — | I | H | — | — | I | A |
| 623 | | | | | | | | | | | | | | |
| Phage- | L | S | — | — | — | — | N | — | — | — | — | — | T | A |
| 624 | | | | | | | | | | | | | | |
| Phage- | N | Y | — | I | — | — | — | — | — | — | — | — | I | D |
| 625 | | | | | | | | | | | | | | |
| Phage- | T | T | — | — | — | — | — | — | V | F | — | — | H | V |
| 626 | | | | | | | | | | | | | | |
| Phage- | F | D | — | — | — | — | R | — | — | — | — | — | N | A |
| 627 | | | | | | | | | | | | | | |
| Phage- | D | — | — | — | — | W | R | — | — | W | — | — | N | N |
| 628 | | | | | | | | | | | | | | |
| Phage- | — | T | — | V | — | I | R | — | — | — | — | — | N | D |
| 629 | | | | | | | | | | | | | | |
| Phage- | — | D | — | L | — | Y | — | — | — | — | — | — | N | Y |
| 630 | | | | | | | | | | | | | | |
| Phage- | L | S | — | — | — | — | Q | — | — | — | — | — | H | A |
| 631 | | | | | | | | | | | | | | |
| Phage- | H | Y | — | M | — | W | Q | — | — | W | — | — | T | V |
| 632 | | | | | | | | | | | | | | |
| Phage- | F | H | — | V | — | W | R | — | — | H | — | — | I | D |
| 633 | | | | | | | | | | | | | | |
| Phage- | V | D | — | — | — | W | R | — | — | — | — | — | N | D |
| 634 | | | | | | | | | | | | | | |
| Phage- | T | A | — | V | — | — | R | — | — | — | — | — | H | V |
| 635 | | | | | | | | | | | | | | |
| Phage- | L | S | — | — | — | — | — | — | — | — | — | — | N | D |
| 636 | | | | | | | | | | | | | | |
| Phage- | — | S | — | — | — | — | — | — | — | — | — | — | H | A |
| 637 | | | | | | | | | | | | | | |
| Phage- | D | — | — | I | — | — | H | — | — | F | — | — | T | V |
| 638 | | | | | | | | | | | | | | |
| Phage- | Y | A | — | — | — | — | R | — | — | — | — | — | N | — |
| 639 | | | | | | | | | | | | | | |
| Phage- | A | S | — | — | — | — | — | — | I | — | — | — | T | V |
| 640 | | | | | | | | | | | | | | |
| Phage- | A | D | — | V | — | — | — | — | — | W | — | — | Y | D |
| 641 | | | | | | | | | | | | | | |
| Phage- | N | T | — | — | — | F | — | — | V | F | — | — | N | V |
| 642 | | | | | | | | | | | | | | |
| Phage- | Y | N | — | — | — | L | Q | — | V | — | — | — | T | I |
| 643 | | | | | | | | | | | | | | |
| Phage- | A | T | — | M | — | — | — | — | — | — | — | — | I | N |
| 644 | | | | | | | | | | | | | | |
| Phage- | H | A | — | L | — | — | E | — | I | — | — | — | Y | A |
| 645 | | | | | | | | | | | | | | |
| Phage- | Y | A | — | — | — | — | N | — | I | — | — | — | T | V |
| 646 | | | | | | | | | | | | | | |
| Phage- | N | T | — | — | — | — | — | — | — | — | — | — | I | V |
| 647 | | | | | | | | | | | | | | |
| Phage- | D | T | — | — | — | — | — | — | — | F | — | — | — | V |
| 648 | | | | | | | | | | | | | | |
| Phage- | N | H | — | V | — | — | R | — | — | W | — | — | N | V |
| 649 | | | | | | | | | | | | | | |
| Phage- | A | S | — | — | — | L | Q | — | — | — | — | — | T | A |
| 650 | | | | | | | | | | | | | | |
| Phage- | F | S | — | — | — | F | — | — | — | — | — | — | N | D |
| 651 | | | | | | | | | | | | | | |
| Phage- | A | S | — | M | — | — | R | — | — | — | — | — | N | A |
| 652 | | | | | | | | | | | | | | |
| Phage- | — | H | — | L | — | F | Q | — | — | — | — | — | T | I |
| 653 | | | | | | | | | | | | | | |
| Phage- | F | S | — | — | — | — | Q | — | — | W | — | — | N | D |
| 654 | | | | | | | | | | | | | | |
| Phage- | — | Y | — | — | — | — | R | — | — | — | — | — | N | S |
| 655 | | | | | | | | | | | | | | |
| Phage- | F | S | — | — | — | — | Q | — | V | F | — | — | T | D |
| 656 | | | | | | | | | | | | | | |
| Phage- | N | H | — | L | — | — | — | — | — | W | — | — | — | V |
| 657 | | | | | | | | | | | | | | |
| Phage- | Y | S | — | — | — | — | E | — | — | — | — | — | N | A |
| 658 | | | | | | | | | | | | | | |
| Phage- | — | H | — | V | — | W | H | — | — | — | — | — | N | D |
| 659 | | | | | | | | | | | | | | |
| Phage- | H | S | — | — | — | — | — | — | — | — | — | — | T | S |
| 660 | | | | | | | | | | | | | | |
| Phage- | D | L | — | W | — | F | Q | — | — | — | — | — | H | V |
| 661 | | | | | | | | | | | | | | |
| Phage- | H | D | — | K | — | F | Q | — | — | — | — | — | Y | D |
| 662 | | | | | | | | | | | | | | |
| Phage- | N | T | — | L | — | W | H | — | — | — | — | — | — | D |
| 663 | | | | | | | | | | | | | | |
| Phage- | F | D | — | V | — | — | R | — | — | — | — | — | T | V |
| 664 | | | | | | | | | | | | | | |
| Phage- | A | T | — | — | — | — | — | — | — | — | — | — | I | D |
| 665 | | | | | | | | | | | | | | |
| Phage- | F | A | — | L | — | F | Q | — | V | — | — | — | H | I |
| 666 | | | | | | | | | | | | | | |
| Phage- | — | S | — | — | — | L | Q | — | — | — | — | — | T | D |
| 667 | | | | | | | | | | | | | | |
| Phage- | N | A | — | W | — | — | — | — | — | — | — | — | I | D |
| 668 | | | | | | | | | | | | | | |
| Phage- | F | A | — | — | — | — | — | — | V | — | — | — | T | N |
| 669 | | | | | | | | | | | | | | |
| Phage- | — | D | — | — | — | — | — | — | I | — | — | — | H | F |
| 670 | | | | | | | | | | | | | | |
| Phage- | N | H | — | L | — | F | Q | — | — | W | — | — | T | V |
| 671 | | | | | | | | | | | | | | |
| Phage- | D | S | — | M | — | W | — | — | — | W | — | — | I | D |
| 672 | | | | | | | | | | | | | | |
| Phage- | V | — | — | — | — | W | Q | — | V | — | — | — | H | S |
| 673 | | | | | | | | | | | | | | |
| Phage- | Y | T | — | Q | — | F | H | — | — | — | — | — | H | I |
| 674 | | | | | | | | | | | | | | |
| Phage- | Y | H | — | L | — | — | — | — | — | — | — | — | I | N |
| 675 | | | | | | | | | | | | | | |
| Phage- | Y | F | — | H | — | F | R | — | — | — | — | — | D | — |
| 676 | | | | | | | | | | | | | | |
| Phage- | D | T | — | L | — | — | — | — | — | F | — | — | I | V |
| 677 | | | | | | | | | | | | | | |
| Phage- | T | L | — | H | — | Y | — | — | — | — | — | — | H | D |
| 678 | | | | | | | | | | | | | | |
| Phage- | Y | L | F | C | I | K | N | L | Y | C | W | N | D | G |
| 679 | | | | | | | | | | | | | | |
| Phage- | L | S | — | — | — | F | — | — | — | — | — | — | N | S |
| 680 | | | | | | | | | | | | | | |
| Phage- | Y | S | — | — | — | — | R | — | — | — | — | — | N | D |
| 681 | | | | | | | | | | | | | | |
| Phage- | N | T | — | — | — | — | — | — | I | — | — | — | — | D |
| 682 | | | | | | | | | | | | | | |
| Phage- | D | S | — | — | — | — | M | — | — | — | — | — | T | D |
| 683 | | | | | | | | | | | | | | |
| Phage- | — | S | — | — | — | I | H | — | — | — | — | — | N | D |
| 684 | | | | | | | | | | | | | | |
| Phage- | — | N | — | V | — | — | R | — | — | — | — | — | I | N |
| 685 | | | | | | | | | | | | | | |
| Phage- | N | S | — | — | — | — | Q | — | — | — | — | — | — | D |
| 686 | | | | | | | | | | | | | | |
| Phage- | A | S | — | — | — | — | H | — | — | — | — | — | T | A |
| 687 | | | | | | | | | | | | | | |
| Phage- | F | S | — | V | — | — | — | — | — | W | — | — | T | V |
| 688 | | | | | | | | | | | | | | |
| Phage- | T | D | — | M | — | F | R | — | — | W | — | — | N | — |
| 689 | | | | | | | | | | | | | | |
| Phage- | — | I | — | L | — | F | Q | — | — | — | — | — | T | — |
| 690 | | | | | | | | | | | | | | |
| Phage- | Y | D | — | — | — | — | R | — | — | W | — | — | N | D |
| 691 | | | | | | | | | | | | | | |
| Phage- | — | N | — | M | — | F | R | — | — | — | — | — | L | D |
| 692 | | | | | | | | | | | | | | |
| Phage- | Y | S | — | H | — | — | R | — | — | — | — | — | H | D |
| 693 | | | | | | | | | | | | | | |
| Phage- | H | T | — | — | — | — | R | — | — | — | — | — | H | D |
| 694 | | | | | | | | | | | | | | |
| Phage- | N | I | — | — | — | — | D | — | — | — | — | — | I | D |
| 695 | | | | | | | | | | | | | | |
| Phage- | A | T | — | M | — | — | M | — | V | — | — | — | Y | A |
| 696 | | | | | | | | | | | | | | |
| Phage- | N | A | — | M | — | — | — | — | V | — | — | — | I | N |
| 697 | | | | | | | | | | | | | | |
| Phage- | D | T | — | V | — | L | R | — | — | F | — | — | I | N |
| 698 | | | | | | | | | | | | | | |
| Phage- | Y | A | — | L | — | L | — | — | I | — | — | — | N | I |
| 699 | | | | | | | | | | | | | | |
| Phage- | N | S | — | V | — | — | M | — | I | — | — | — | H | — |
| 700 | | | | | | | | | | | | | | |
| Phage- | N | — | — | — | — | M | D | — | — | W | — | — | I | D |
| 701 | | | | | | | | | | | | | | |
| Phage- | H | N | — | V | — | L | Q | — | — | — | — | — | N | D |
| 702 | | | | | | | | | | | | | | |
| Phage- | N | A | — | M | — | F | — | — | V | — | — | — | I | D |
| 703 | | | | | | | | | | | | | | |
| Phage- | V | F | — | M | — | — | R | — | — | — | — | — | H | D |
| 704 | | | | | | | | | | | | | | |
| Phage- | D | Y | — | V | — | — | R | — | — | — | — | — | I | N |
| 705 | | | | | | | | | | | | | | |
| Phage- | F | S | — | M | — | E | — | — | — | W | — | — | H | V |
| 706 | | | | | | | | | | | | | | |
| Phage- | Y | T | — | — | — | — | N | — | I | — | — | — | T | D |
| 707 | | | | | | | | | | | | | | |
| Phage- | N | F | — | V | — | — | — | — | — | — | — | — | I | D |
| 708 | | | | | | | | | | | | | | |
| Phage- | F | D | — | — | — | — | R | — | — | — | — | — | N | D |
| 709 | | | | | | | | | | | | | | |
| Phage- | H | T | — | — | — | — | R | — | — | — | — | — | T | I |
| 710 | | | | | | | | | | | | | | |
| Phage- | D | — | — | L | — | — | — | — | — | F | — | — | T | V |
| 711 | | | | | | | | | | | | | | |
| Phage- | N | T | — | V | — | F | Q | — | F | F | — | — | I | D |
| 712 | | | | | | | | | | | | | | |
| Phage- | N | S | — | — | — | — | — | — | — | W | — | — | I | A |
| 713 | | | | | | | | | | | | | | |
| Phage- | F | A | — | — | — | — | — | — | — | — | — | — | H | I |
| 714 | | | | | | | | | | | | | | |
| Phage- | D | N | — | — | — | I | R | — | — | — | — | — | I | D |
| 715 | | | | | | | | | | | | | | |
| Phage- | H | S | — | — | — | F | Q | — | — | W | — | — | H | D |
| 716 | | | | | | | | | | | | | | |
| Phage- | — | I | — | W | — | F | Q | — | — | — | — | — | H | V |
| 717 | | | | | | | | | | | | | | |
| Phage- | — | D | — | V | — | — | M | — | I | — | — | F | H | — |
| 718 | | | | | | | | | | | | | | |
| Phage- | D | H | — | V | — | W | R | — | V | — | — | — | H | — |
| 719 | | | | | | | | | | | | | | |
| Phage- | F | S | — | — | — | — | — | — | — | — | — | — | T | D |
| 720 | | | | | | | | | | | | | | |
| Phage- | D | H | — | V | — | — | — | — | — | L | — | — | I | V |
| 721 | | | | | | | | | | | | | | |
| Phage- | N | I | — | L | — | — | — | — | — | — | — | — | N | D |
| 722 | | | | | | | | | | | | | | |
| Phage- | D | Y | — | M | — | — | — | — | — | F | — | — | I | I |
| 723 | | | | | | | | | | | | | | |
| Phage- | N | F | — | V | — | — | — | — | — | — | — | — | I | S |
| 724 | | | | | | | | | | | | | | |
| Phage- | T | H | — | L | — | — | M | — | I | — | — | Y | H | I |
| 725 | | | | | | | | | | | | | | |
| Phage- | Y | T | F | — | — | — | — | — | — | — | — | — | N | D |
| 726 | | | | | | | | | | | | | | |
| Phage- | D | — | — | — | — | — | Q | — | V | — | — | — | H | I |
| 727 | | | | | | | | | | | | | | |
| Phage- | D | L | — | V | — | — | — | — | — | F | — | — | T | V |
| 728 | | | | | | | | | | | | | | |
| Phage- | D | I | — | — | — | F | Q | — | I | — | — | — | H | — |
| 729 | | | | | | | | | | | | | | |
| Phage- | Y | N | — | L | — | — | — | — | I | — | — | — | H | D |
| 730 | | | | | | | | | | | | | | |
| Phage- | N | F | — | V | — | W | — | — | T | L | — | — | I | A |
| 731 | | | | | | | | | | | | | | |
| Phage- | D | S | — | — | — | L | R | — | — | — | — | — | T | A |
| 732 | | | | | | | | | | | | | | |
| Phage- | Y | S | — | M | — | — | — | — | — | — | — | — | N | D |
| 733 | | | | | | | | | | | | | | |
| Phage- | D | S | — | — | — | — | R | — | — | F | — | — | T | — |
| 734 | | | | | | | | | | | | | | |
| Phage- | D | A | — | — | — | I | — | — | — | W | — | — | N | S |
| 735 | | | | | | | | | | | | | | |
| Phage- | N | Y | — | I | — | — | — | — | — | — | — | — | I | N |
| 736 | | | | | | | | | | | | | | |
| Phage- | F | D | — | V | — | — | R | — | — | W | — | — | H | — |
| 737 | | | | | | | | | | | | | | |
| Phage- | Y | D | — | L | — | F | — | — | — | W | — | — | N | — |
| 738 | | | | | | | | | | | | | | |
| Phage- | D | F | — | R | — | — | — | — | — | — | — | — | I | D |
| 739 | | | | | | | | | | | | | | |
| Phage- | N | A | — | V | — | I | — | — | — | W | — | — | H | V |
| 740 | | | | | | | | | | | | | | |
| Phage- | A | A | — | V | — | — | — | — | I | — | — | — | I | H |
| 741 | | | | | | | | | | | | | | |
| Phage- | N | S | — | L | — | — | R | — | — | — | — | — | I | A |
| 742 | | | | | | | | | | | | | | |
| Phage- | N | L | — | — | — | F | Q | — | — | — | — | — | I | D |
| 743 | | | | | | | | | | | | | | |
| Phage- | T | T | — | — | — | — | — | — | — | — | — | — | H | A |
| 744 | | | | | | | | | | | | | | |
| Phage- | D | F | M | V | — | — | — | — | — | F | — | — | I | D |
| 745 | | | | | | | | | | | | | | |
| Phage- | T | S | — | V | — | W | R | — | V | — | — | — | H | D |
| 746 | | | | | | | | | | | | | | |
| Phage- | Y | S | — | — | — | — | — | — | — | — | — | — | N | D |
| 747 | | | | | | | | | | | | | | |
| Phage- | — | Y | — | — | — | F | Q | — | — | — | — | — | N | I |
| 748 | | | | | | | | | | | | | | |
| Phage- | D | T | — | V | — | I | R | — | — | — | — | — | I | D |
| 749 | | | | | | | | | | | | | | |
| Phage- | N | T | — | — | — | — | — | — | — | — | — | — | T | A |
| 750 | | | | | | | | | | | | | | |
| Phage- | N | L | — | — | — | I | N | — | — | F | — | — | N | D |
| 751 | | | | | | | | | | | | | | |
| Phage- | F | S | — | N | — | — | — | — | I | — | — | — | H | — |
| 752 | | | | | | | | | | | | | | |
| Phage- | H | A | — | — | — | — | — | — | — | W | — | — | H | D |
| 753 | | | | | | | | | | | | | | |
| Phage- | N | H | — | V | — | F | Q | — | V | K | — | — | I | A |
| 754 | | | | | | | | | | | | | | |
| Phage- | N | D | — | — | — | — | Q | — | I | W | — | — | H | D |
| 755 | | | | | | | | | | | | | | |
| Phage- | N | D | — | I | — | — | — | — | — | W | — | — | H | — |
| 756 | | | | | | | | | | | | | | |
| Phage- | D | T | — | V | — | — | R | — | — | F | — | — | I | Y |
| 757 | | | | | | | | | | | | | | |
| Phage- | Y | I | — | H | — | F | R | — | — | — | — | — | N | — |
| 758 | | | | | | | | | | | | | | |
| Phage- | H | — | — | L | — | W | R | — | V | — | — | — | H | S |
| 759 | | | | | | | | | | | | | | |
| Phage- | F | A | — | — | — | — | R | — | I | — | — | — | H | A |
| 760 | | | | | | | | | | | | | | |
| Phage- | N | T | — | M | — | — | — | — | I | — | — | — | I | I |
| 761 | | | | | | | | | | | | | | |
| Phage- | Y | S | — | — | — | — | H | — | — | — | — | — | H | V |
| 762 | | | | | | | | | | | | | | |
| Phage- | N | T | — | V | — | — | R | — | — | — | — | — | I | H |
| 763 | | | | | | | | | | | | | | |
| Phage- | N | N | — | — | — | — | — | — | — | — | — | — | T | A |
| 764 | | | | | | | | | | | | | | |
| Phage- | — | D | — | — | — | — | R | — | — | — | — | — | H | Y |
| 765 | | | | | | | | | | | | | | |
| Phage- | H | A | — | — | — | — | — | — | — | — | — | — | T | A |
| 766 | | | | | | | | | | | | | | |
| Phage- | D | T | — | L | — | — | R | — | — | W | — | — | I | D |
| 767 | | | | | | | | | | | | | | |
| Phage- | D | S | — | L | — | — | — | — | — | F | — | — | I | V |
| 768 | | | | | | | | | | | | | | |
| Phage- | D | H | — | V | — | — | R | — | — | F | — | — | I | — |
| 769 | | | | | | | | | | | | | | |
| Phage- | D | T | — | L | — | — | — | — | — | F | — | — | T | Y |
| 770 | | | | | | | | | | | | | | |
| Phage- | D | S | — | — | — | — | — | — | — | F | — | — | P | V |
| 771 | | | | | | | | | | | | | | |
| Phage- | Y | D | — | — | — | W | — | — | — | — | — | — | N | — |
| 772 | | | | | | | | | | | | | | |
| Phage- | N | D | — | — | — | — | R | — | — | — | — | — | N | A |
| 773 | | | | | | | | | | | | | | |
| Phage- | N | I | — | — | — | F | Q | — | — | — | — | — | H | A |
| 774 | | | | | | | | | | | | | | |
| Phage- | — | H | — | L | — | — | — | — | I | F | — | — | T | A |
| 775 | | | | | | | | | | | | | | |
| Phage- | Y | A | — | — | — | L | — | — | — | — | — | — | N | V |
| 776 | | | | | | | | | | | | | | |
| Phage- | N | A | — | M | — | — | — | — | — | — | — | — | I | H |
| 777 | | | | | | | | | | | | | | |
| Phage- | A | N | — | L | — | F | H | — | — | W | — | — | H | D |
| 778 | | | | | | | | | | | | | | |
| Phage- | F | S | — | — | — | — | Q | — | V | — | — | — | T | S |
| 779 | | | | | | | | | | | | | | |
| Phage- | L | H | — | — | — | — | — | — | — | — | — | — | H | A |
| 780 | | | | | | | | | | | | | | |
| Phage- | H | N | — | I | — | — | — | — | — | W | — | — | H | N |
| 781 | | | | | | | | | | | | | | |
| Phage- | N | T | — | V | — | F | N | — | — | — | — | Y | I | D |
| 782 | | | | | | | | | | | | | | |
| Phage- | F | S | — | — | — | — | N | — | — | W | — | — | N | D |
| 783 | | | | | | | | | | | | | | |
| Phage- | — | Y | — | — | — | — | — | — | I | — | — | — | N | F |
| 784 | | | | | | | | | | | | | | |
| Phage- | H | S | — | — | — | — | — | — | — | — | — | — | T | A |
| 785 | | | | | | | | | | | | | | |
| Phage- | L | S | — | — | — | — | R | — | — | — | — | — | T | A |
| 786 | | | | | | | | | | | | | | |
| Phage- | A | A | — | I | — | — | — | — | — | — | — | — | N | — |
| 787 | | | | | | | | | | | | | | |
| Phage- | — | S | — | W | — | F | Q | — | V | — | — | — | — | V |
| 788 | | | | | | | | | | | | | | |
| Phage- | A | — | — | W | — | F | Q | — | V | — | — | — | N | I |
| 789 | | | | | | | | | | | | | | |
| Phage- | N | S | — | — | — | — | R | — | — | — | — | — | I | A |
| 790 | | | | | | | | | | | | | | |
| Phage- | Y | Y | — | N | — | I | — | — | — | — | — | — | T | D |
| 791 | | | | | | | | | | | | | | |
| Phage- | Y | S | — | — | — | L | R | — | — | — | — | — | T | D |
| 792 | | | | | | | | | | | | | | |
| Phage- | F | A | — | — | — | — | N | — | — | — | — | — | T | S |
| 793 | | | | | | | | | | | | | | |
| Phage- | N | F | — | K | — | F | Q | — | V | — | — | — | H | V |
| 794 | | | | | | | | | | | | | | |
| Phage- | D | S | — | M | — | L | R | — | — | F | — | — | T | Y |
| 795 | | | | | | | | | | | | | | |
| Phage- | N | Y | — | V | — | — | — | — | I | — | — | — | I | H |
| 796 | | | | | | | | | | | | | | |
| Phage- | Y | S | — | V | — | — | — | — | — | — | — | — | N | V |
| 797 | | | | | | | | | | | | | | |
| Phage- | — | F | — | W | — | F | Q | — | — | — | — | — | N | D |
| 798 | | | | | | | | | | | | | | |
| Phage- | N | T | — | — | — | — | R | — | — | — | — | — | I | D |
| 799 | | | | | | | | | | | | | | |
| Phage- | D | S | — | R | — | — | N | — | — | — | — | — | I | D |
| 800 | | | | | | | | | | | | | | |
| Phage- | A | D | — | V | — | I | M | — | I | — | — | Y | N | — |
| 801 | | | | | | | | | | | | | | |
| Phage- | N | N | — | V | — | — | — | — | I | — | — | — | I | F |
| 802 | | | | | | | | | | | | | | |
| Phage- | — | D | — | M | — | L | N | — | — | W | — | — | H | — |
| 803 | | | | | | | | | | | | | | |
| Phage- | N | F | — | W | — | — | — | — | — | W | — | — | T | S |
| 804 | | | | | | | | | | | | | | |
| Phage- | Y | L | V | — | — | — | D | K | R | K | — | M | — | L |
| 805 | | | | | | | | | | | | | | |
| Phage- | A | A | — | — | — | W | — | — | — | — | — | — | H | I |
| 806 | | | | | | | | | | | | | | |
| Phage- | D | L | — | L | — | F | R | — | — | W | — | — | H | V |
| 807 | | | | | | | | | | | | | | |
| Phage- | D | — | — | V | — | L | R | — | — | F | — | — | I | D |
| 808 | | | | | | | | | | | | | | |
| Phage- | F | S | — | H | — | — | M | — | — | — | — | — | H | V |
| 809 | | | | | | | | | | | | | | |
| Phage- | N | A | — | I | — | — | — | — | — | W | — | — | N | — |
| 810 | | | | | | | | | | | | | | |
| Phage- | — | D | — | — | — | F | R | — | — | — | — | — | H | D |
| 811 | | | | | | | | | | | | | | |
| Phage- | D | A | — | V | — | — | — | — | — | F | — | — | I | D |
| 812 | | | | | | | | | | | | | | |
| Phage- | N | F | — | M | — | F | H | — | — | — | — | — | I | N |
| 813 | | | | | | | | | | | | | | |
| Phage- | A | S | — | Y | — | — | Q | — | — | F | — | — | H | S |
| 814 | | | | | | | | | | | | | | |
| Phage- | N | S | — | — | — | — | R | — | — | — | — | — | T | D |
| 815 | | | | | | | | | | | | | | |
| Phage- | N | H | — | M | — | I | — | — | — | — | — | — | I | D |
| 816 | | | | | | | | | | | | | | |
| Phage- | N | H | — | — | — | I | N | — | — | — | — | — | H | S |
| 817 | | | | | | | | | | | | | | |
| Phage- | F | S | — | — | — | I | — | — | — | — | — | — | H | — |
| 818 | | | | | | | | | | | | | | |
| Phage- | N | A | — | — | — | M | E | — | — | — | — | — | I | D |
| 819 | | | | | | | | | | | | | | |
| Phage- | — | A | — | — | — | F | Q | — | — | — | — | — | H | F |
| 820 | | | | | | | | | | | | | | |
| Phage- | D | S | — | V | — | — | R | — | — | L | — | — | I | V |
| 821 | | | | | | | | | | | | | | |
| Phage- | L | A | — | — | — | L | Q | — | I | — | — | — | H | S |
| 822 | | | | | | | | | | | | | | |
| Phage- | Y | T | — | M | — | — | — | — | — | — | — | — | H | V |
| 823 | | | | | | | | | | | | | | |
| Phage- | D | T | — | W | — | — | H | — | V | — | — | — | I | I |
| 824 | | | | | | | | | | | | | | |
| Phage- | N | S | — | — | — | — | — | — | I | — | — | — | N | I |
| 825 | | | | | | | | | | | | | | |
| Phage- | N | D | — | L | — | F | R | — | — | W | — | — | Y | D |
| 826 | | | | | | | | | | | | | | |
| Phage- | T | D | — | L | — | F | R | — | — | W | — | — | H | S |
| 827 | | | | | | | | | | | | | | |
| Phage- | N | T | — | L | — | L | N | — | — | F | — | — | I | D |
| 828 | | | | | | | | | | | | | | |
| Phage- | — | H | — | — | — | L | R | — | — | — | — | — | H | V |
| 829 | | | | | | | | | | | | | | |
| Phage- | N | — | — | — | — | F | Q | — | I | — | — | — | N | F |
| 830 | | | | | | | | | | | | | | |
| Phage- | V | H | — | M | — | — | — | — | — | W | — | — | H | A |
| 831 | | | | | | | | | | | | | | |
| Phage- | D | L | — | V | — | — | R | — | — | F | — | — | I | D |
| 832 | | | | | | | | | | | | | | |
| Phage- | N | T | — | L | — | — | — | — | — | — | — | — | I | V |
| 833 | | | | | | | | | | | | | | |
| Phage- | D | T | — | I | — | — | R | — | — | — | — | — | I | D |
| 834 | | | | | | | | | | | | | | |
| Phage- | Y | A | — | — | — | — | R | — | — | — | — | Y | N | A |
| 835 | | | | | | | | | | | | | | |
| Phage- | N | A | — | V | — | F | Q | — | I | — | — | — | I | D |
| 836 | | | | | | | | | | | | | | |
| Phage- | N | A | — | — | — | — | — | — | I | F | — | — | N | S |
| 837 | | | | | | | | | | | | | | |
| Phage- | — | T | — | — | — | W | R | — | — | V | — | — | T | A |
| 838 | | | | | | | | | | | | | | |
| Phage- | N | D | — | L | — | F | Q | — | — | W | — | — | H | A |
| 839 | | | | | | | | | | | | | | |
| Phage- | N | F | — | V | — | — | — | — | F | — | — | — | I | D |
| 840 | | | | | | | | | | | | | | |
| Phage- | — | I | — | M | — | — | — | — | — | W | — | — | I | D |
| 841 | | | | | | | | | | | | | | |
| Phage- | D | H | — | — | — | F | R | — | — | W | — | — | N | I |
| 842 | | | | | | | | | | | | | | |
| Phage- | Y | D | — | I | — | F | R | — | I | W | — | — | N | D |
| 843 | | | | | | | | | | | | | | |
| Phage- | D | A | — | — | — | — | — | — | — | F | — | — | T | Y |
| 844 | | | | | | | | | | | | | | |
| Phage- | N | D | — | I | — | — | — | — | I | — | — | — | H | F |
| 845 | | | | | | | | | | | | | | |
| Phage- | F | S | — | — | — | — | — | — | — | — | — | — | N | A |
| 846 | | | | | | | | | | | | | | |
| Phage- | I | F | A | L | — | W | L | K | — | W | — | L | I | N |
| 847 | | | | | | | | | | | | | | |
| Phage- | D | T | — | — | — | L | R | — | V | L | — | — | T | I |
| 848 | | | | | | | | | | | | | | |
| Phage- | A | T | — | Q | — | F | — | — | I | — | — | — | I | I |
| 849 | | | | | | | | | | | | | | |
| Phage- | H | S | — | — | — | I | — | — | — | W | — | — | N | H |
| 850 | | | | | | | | | | | | | | |
| Phage- | — | A | — | Y | — | F | R | — | V | F | — | — | H | V |
| 851 | | | | | | | | | | | | | | |
| Phage- | D | S | — | V | — | — | — | — | — | L | — | — | I | Y |
| 852 | | | | | | | | | | | | | | |
| Phage- | A | — | — | Y | — | F | Q | — | I | — | — | — | H | I |
| 853 | | | | | | | | | | | | | | |
| Phage- | — | D | — | — | — | W | R | — | — | W | — | — | N | D |
| 854 | | | | | | | | | | | | | | |
| Phage- | A | S | — | — | — | — | — | — | — | — | — | — | T | S |
| 855 | | | | | | | | | | | | | | |
| Phage- | D | A | — | M | — | — | — | — | — | L | — | — | I | V |
| 856 | | | | | | | | | | | | | | |
| Phage- | D | T | — | R | — | — | M | — | I | — | — | — | I | D |
| 857 | | | | | | | | | | | | | | |
| Phage- | D | T | — | — | — | — | — | — | — | F | — | — | T | Y |
| 858 | | | | | | | | | | | | | | |
| Phage- | F | S | — | — | — | — | N | — | V | — | — | — | T | D |
| 859 | | | | | | | | | | | | | | |
| Phage- | N | S | — | M | — | — | M | — | — | — | — | — | T | I |
| 860 | | | | | | | | | | | | | | |
| Phage- | H | I | — | — | — | Q | H | K | R | A | — | Q | I | S |
| 861 | | | | | | | | | | | | | | |
| Phage- | D | H | — | M | — | — | N | — | W | F | — | — | I | N |
| 862 | | | | | | | | | | | | | | |
| Phage- | D | S | — | — | — | F | R | — | — | W | — | — | — | I |
| 863 | | | | | | | | | | | | | | |
| Phage- | — | S | — | M | — | — | R | — | V | — | — | — | I | N |
| 864 | | | | | | | | | | | | | | |
| Phage- | Y | H | — | — | — | — | — | — | I | — | — | — | H | V |
| 865 | | | | | | | | | | | | | | |
| Phage- | D | S | — | — | — | I | R | — | — | F | — | — | T | A |
| 866 | | | | | | | | | | | | | | |
| Phage- | N | T | — | L | — | W | H | — | — | — | — | — | I | N |
| 867 | | | | | | | | | | | | | | |
| Phage- | T | A | — | I | — | — | — | — | — | — | — | — | I | D |
| 868 | | | | | | | | | | | | | | |
| Phage- | F | S | — | L | — | F | Q | — | I | — | — | — | T | V |
| 869 | | | | | | | | | | | | | | |
| Phage- | D | T | — | H | — | — | — | — | I | F | — | — | T | D |
| 870 | | | | | | | | | | | | | | |
| Phage- | N | T | — | V | — | F | — | — | V | H | — | — | I | D |
| 871 | | | | | | | | | | | | | | |
| Phage- | — | N | — | L | — | — | M | — | I | — | — | — | H | V |
| 872 | | | | | | | | | | | | | | |
| Phage- | A | S | — | I | — | — | N | — | — | — | — | — | I | D |
| 873 | | | | | | | | | | | | | | |
| Phage- | N | D | — | V | — | — | — | — | — | W | — | — | N | S |
| 874 | | | | | | | | | | | | | | |
| Phage- | H | F | — | M | — | — | H | — | V | F | — | — | I | S |
| 875 | | | | | | | | | | | | | | |
| Phage- | A | T | P | S | — | — | Q | — | V | Q | — | Y | I | V |
| 876 | | | | | | | | | | | | | | |
| Phage- | T | H | — | — | — | — | R | — | — | — | — | — | H | A |
| 877 | | | | | | | | | | | | | | |
| Phage- | Y | D | — | — | — | I | R | — | I | — | — | — | T | Y |
| 878 | | | | | | | | | | | | | | |
| Phage- | F | D | — | L | — | — | — | — | I | — | — | — | H | D |
| 879 | | | | | | | | | | | | | | |
| Phage- | D | S | — | Y | — | — | — | — | — | F | — | — | T | F |
| 880 | | | | | | | | | | | | | | |
| Phage- | N | H | — | V | — | — | — | — | — | W | — | — | I | Y |
| 881 | | | | | | | | | | | | | | |
| Phage- | N | H | — | V | — | — | R | — | — | — | — | — | I | — |
| 882 | | | | | | | | | | | | | | |
| Phage- | — | F | — | V | — | — | — | — | — | — | — | — | T | D |
| 883 | | | | | | | | | | | | | | |
| Phage- | H | S | — | — | — | — | R | — | — | — | — | — | N | A |
| 884 | | | | | | | | | | | | | | |
| Phage- | N | — | — | Y | — | — | — | — | I | — | — | — | N | S |
| 885 | | | | | | | | | | | | | | |
| Phage- | D | Y | — | — | — | F | Q | — | V | — | — | — | I | A |
| 886 | | | | | | | | | | | | | | |
| Phage- | N | Y | — | — | — | L | R | — | — | — | — | — | T | A |
| 887 | | | | | | | | | | | | | | |
| Phage- | L | S | — | — | — | W | H | — | — | — | — | — | N | I |
| 888 | | | | | | | | | | | | | | |
| Phage- | N | T | — | — | — | F | Q | — | — | — | — | — | I | N |
| 889 | | | | | | | | | | | | | | |
| Phage- | F | S | — | — | — | — | N | — | — | W | — | — | T | D |
| 890 | | | | | | | | | | | | | | |
| Phage- | F | S | — | — | — | L | N | — | — | — | — | — | N | A |
| 891 | | | | | | | | | | | | | | |
| Phage- | Y | S | — | M | — | — | — | — | — | W | — | — | N | D |
| 892 | | | | | | | | | | | | | | |
| Phage- | N | A | — | W | — | — | — | — | V | — | — | — | T | S |
| 893 | | | | | | | | | | | | | | |
| Phage- | N | P | — | Q | — | S | W | K | Y | Q | — | Q | — | F |
| 894 | | | | | | | | | | | | | | |
| Phage- | N | H | — | — | — | — | M | — | I | — | — | — | N | — |
| 895 | | | | | | | | | | | | | | |
| Phage- | N | H | — | H | — | — | M | — | V | — | — | — | I | V |
| 896 | | | | | | | | | | | | | | |
| Phage- | F | D | — | I | — | W | — | — | — | W | — | — | N | D |
| 897 | | | | | | | | | | | | | | |
| Phage- | — | F | — | Q | — | L | M | — | I | — | — | — | H | D |
| 898 | | | | | | | | | | | | | | |
| Phage- | Y | S | — | — | — | L | — | — | — | — | — | — | N | V |
| 899 | | | | | | | | | | | | | | |
| Phage- | N | A | — | — | — | — | R | — | — | — | — | — | T | — |
| 900 | | | | | | | | | | | | | | |
| Phage- | — | D | — | — | — | F | R | — | — | W | — | — | N | S |
| 901 | | | | | | | | | | | | | | |
| Phage- | V | — | — | V | — | L | — | — | — | Q | — | F | D | D |
| 902 | | | | | | | | | | | | | | |
| Phage- | A | D | — | L | — | W | R | — | — | W | — | — | S | A |
| 903 | | | | | | | | | | | | | | |
| Phage- | F | S | — | — | — | — | — | — | I | — | — | — | — | A |
| 904 | | | | | | | | | | | | | | |
| Phage- | D | S | — | — | — | I | — | — | V | F | — | — | T | Y |
| 905 | | | | | | | | | | | | | | |
| Phage- | — | T | — | Y | — | I | — | — | I | W | — | — | N | V |
| 906 | | | | | | | | | | | | | | |
| Phage- | Y | S | — | — | — | — | — | — | V | — | — | — | T | S |
| 907 | | | | | | | | | | | | | | |
| Phage- | N | T | — | I | — | — | R | — | — | — | — | — | — | Y |
| 908 | | | | | | | | | | | | | | |
| Phage- | D | N | — | V | — | — | — | — | — | F | — | — | I | V |
| 909 | | | | | | | | | | | | | | |
| Phage- | D | S | — | W | — | — | R | — | — | — | — | — | I | V |
| 910 | | | | | | | | | | | | | | |
| Phage- | D | Y | — | H | — | — | R | — | — | F | — | — | I | — |
| 911 | | | | | | | | | | | | | | |
| Phage- | H | I | — | S | — | W | H | — | I | — | — | — | Y | D |
| 912 | | | | | | | | | | | | | | |
| Phage- | Y | S | — | — | — | L | R | — | — | — | — | — | N | Y |
| 913 | | | | | | | | | | | | | | |
| Phage- | — | P | Y | L | — | K | Q | K | Q | L | — | G | S | I |
| 914 | | | | | | | | | | | | | | |
| Phage- | F | S | — | — | — | — | — | — | V | — | — | — | T | A |
| 915 | | | | | | | | | | | | | | |
| Phage- | N | A | — | — | — | — | R | — | — | — | — | — | I | S |
| 916 | | | | | | | | | | | | | | |
| Phage- | Y | S | — | — | — | — | — | — | — | W | — | — | N | A |
| 917 | | | | | | | | | | | | | | |
| Phage- | Y | S | — | — | — | — | — | — | — | W | — | — | T | D |
| 918 | | | | | | | | | | | | | | |
| Phage- | N | H | P | Q | — | — | N | K | — | Q | — | — | D | H |
| 919 | | | | | | | | | | | | | | |
| Phage- | H | D | — | — | — | F | R | — | — | W | — | — | T | V |
| 920 | | | | | | | | | | | | | | |
| Phage- | A | Y | — | L | — | — | M | — | — | W | — | T | N | V |
| 921 | | | | | | | | | | | | | | |
| Phage- | Y | A | — | Y | — | F | Q | — | — | W | — | — | H | N |
| 922 | | | | | | | | | | | | | | |
| Phage- | D | H | — | W | — | F | R | — | — | W | — | — | T | I |
| 923 | | | | | | | | | | | | | | |
| Phage- | D | T | — | W | — | — | R | — | — | F | — | — | T | — |
| 924 | | | | | | | | | | | | | | |
| Phage- | H | T | — | W | — | F | R | — | — | — | — | — | N | I |
| 925 | | | | | | | | | | | | | | |
| Phage- | H | S | — | — | — | — | H | — | — | W | — | — | T | I |
| 926 | | | | | | | | | | | | | | |
| Phage- | N | Y | — | L | — | F | H | — | — | — | — | — | I | S |
| 927 | | | | | | | | | | | | | | |
| Phage- | V | — | — | V | — | — | D | H | R | Q | — | — | H | D |
| 928 | | | | | | | | | | | | | | |
| Phage- | N | S | — | — | — | — | N | — | I | — | — | — | N | A |
| 929 | | | | | | | | | | | | | | |
| Phage- | — | H | — | W | — | F | R | — | I | W | — | — | — | — |
| 930 | | | | | | | | | | | | | | |
| Phage- | F | S | — | — | — | — | — | — | — | — | — | — | N | S |
| 931 | | | | | | | | | | | | | | |
| Phage- | L | L | P | V | L | C | Q | E | Y | V | V | C | G | R |
| 932 | | | | | | | | | | | | | | |
| Phage- | — | Y | — | — | — | F | Q | — | V | — | — | — | N | V |
| 933 | | | | | | | | | | | | | | |
| Phage- | H | N | — | L | — | G | Q | K | — | — | — | Y | D | L |
| 934 | | | | | | | | | | | | | | |
| Phage- | D | H | R | Y | — | — | N | D | Q | Q | — | — | — | P |
| 935 | | | | | | | | | | | | | | |
| Phage- | H | A | — | — | — | L | — | — | — | W | — | — | N | I |
| 936 | | | | | | | | | | | | | | |
| Phage- | Y | H | — | — | — | F | R | — | V | — | — | — | H | V |
| 937 | | | | | | | | | | | | | | |
| Phage- | T | F | — | W | — | F | Q | — | V | — | — | — | N | A |
| 938 | | | | | | | | | | | | | | |
| Phage- | F | S | — | — | — | F | Q | — | — | W | — | — | N | S |
| 939 | | | | | | | | | | | | | | |
| Phage- | L | I | — | — | — | — | T | — | — | Q | — | S | I | H |
| 940 | | | | | | | | | | | | | | |
| Phage- | D | Y | — | M | — | L | R | — | V | F | — | — | T | V |
| 941 | | | | | | | | | | | | | | |
| Phage- | D | A | F | Q | — | G | E | R | — | Q | — | — | N | D |
| 942 | | | | | | | | | | | | | | |
| Phage- | H | S | — | — | — | — | R | — | — | W | — | — | T | V |
| 943 | | | | | | | | | | | | | | |
| Phage- | L | D | — | I | — | — | — | — | — | — | — | — | I | N |
| 944 | | | | | | | | | | | | | | |
| Phage- | F | D | — | — | — | R | R | K | — | W | — | P | — | N |
| 945 | | | | | | | | | | | | | | |
| Phage- | Y | T | — | — | — | W | R | — | V | H | — | — | H | I |
| 946 | | | | | | | | | | | | | | |
| Phage- | — | H | — | W | — | F | R | — | — | — | — | — | D | V |
| 947 | | | | | | | | | | | | | | |
| Phage- | D | T | — | — | — | — | R | — | I | — | — | — | H | I |
| 948 | | | | | | | | | | | | | | |
| Phage- | H | N | — | L | — | W | R | — | I | — | — | — | I | D |
| 949 | | | | | | | | | | | | | | |
| Phage- | D | I | — | — | — | L | R | — | V | F | — | — | T | A |
| 950 | | | | | | | | | | | | | | |
| Phage- | P | — | V | L | — | L | H | K | H | Q | — | L | S | Y |
| 951 | | | | | | | | | | | | | | |
| Phage- | N | I | — | L | — | — | N | K | M | Q | — | F | Y | H |
| 952 | | | | | | | | | | | | | | |
| Phage- | A | D | — | W | — | — | R | — | — | — | — | — | I | I |
| 953 | | | | | | | | | | | | | | |
| Phage- | H | — | — | — | — | W | H | — | V | — | — | — | H | — |
| 954 | | | | | | | | | | | | | | |
| Phage- | A | F | — | M | — | W | H | — | — | — | — | — | H | — |
| 955 | | | | | | | | | | | | | | |
| Phage- | V | I | — | L | — | S | D | Q | — | — | — | K | D | — |
| 956 | | | | | | | | | | | | | | |
| Phage- | V | Y | — | — | — | — | Q | E | — | K | — | — | F | S |
| 957 | | | | | | | | | | | | | | |
| Phage- | I | H | — | W | — | F | R | — | V | — | — | — | H | V |
| 958 | | | | | | | | | | | | | | |
| Phage- | D | I | — | — | — | W | R | — | — | — | — | — | T | S |
| 959 | | | | | | | | | | | | | | |
| Phage- | F | D | — | — | — | — | R | — | — | W | — | — | N | A |
| 960 | | | | | | | | | | | | | | |
| Phage- | Y | S | — | — | — | — | — | — | — | W | — | — | N | V |
| 961 | | | | | | | | | | | | | | |
| Phage- | N | Y | — | W | — | W | — | — | — | — | — | — | T | D |
| 962 | | | | | | | | | | | | | | |
| Phage- | Y | — | — | H | — | W | H | — | — | — | — | — | N | — |
| 963 | | | | | | | | | | | | | | |
| Phage- | D | — | S | — | — | Q | Q | K | — | Q | — | S | Y | D |
| 964 | | | | | | | | | | | | | | |
| Phage- | N | D | — | V | — | W | R | — | I | — | — | — | I | Y |
| 965 | | | | | | | | | | | | | | |
| Phage- | N | T | V | P | — | A | M | — | — | W | — | R | D | F |
| 966 | | | | | | | | | | | | | | |
| Phage- | — | H | F | — | — | W | R | — | — | — | — | — | N | D |
| 967 | | | | | | | | | | | | | | |
| Phage- | D | F | — | — | — | L | R | — | V | F | — | — | T | A |
| 968 | | | | | | | | | | | | | | |
| Phage- | N | D | — | — | — | — | Q | — | — | — | — | — | H | — |
| 969 | | | | | | | | | | | | | | |
| Phage- | D | A | — | — | — | F | R | — | — | W | — | — | N | L |
| 970 | | | | | | | | | | | | | | |
| Phage- | Y | N | — | V | — | F | Q | — | I | F | — | — | H | D |
| 971 | | | | | | | | | | | | | | |
| Phage- | V | P | — | — | — | — | L | K | I | F | — | G | H | N |
| 972 | | | | | | | | | | | | | | |
| Phage- | Y | S | — | — | — | — | H | — | — | — | — | — | N | A |
| 973 | | | | | | | | | | | | | | |
| Phage- | T | N | — | — | — | G | — | S | — | Q | — | R | N | I |
| 974 | | | | | | | | | | | | | | |
| Phage- | H | I | V | — | — | — | N | Y | — | Q | — | G | H | F |
| 975 | | | | | | | | | | | | | | |
| Phage- | N | H | — | L | — | A | S | Q | F | Q | — | — | N | A |
| 976 | | | | | | | | | | | | | | |
| Phage- | F | I | — | — | — | G | Q | H | — | Q | — | L | D | A |
| 977 | | | | | | | | | | | | | | |
| Phage- | A | D | — | L | — | F | R | — | — | W | — | — | H | N |
| 978 | | | | | | | | | | | | | | |
| Phage- | T | T | — | W | — | Y | R | — | V | — | — | — | N | I |
| 979 | | | | | | | | | | | | | | |
| Phage- | D | S | — | W | — | L | — | — | — | V | — | Q | S | N |
| 980 | | | | | | | | | | | | | | |

| | Phage binding ELISA | | | |
|---|---|---|---|---|
| | | | TROP2 Fab | |
| | | TROP2 | signal in | |
| | Backgroud | Fab | presence of | SEQ |
| | signal | signal | TROP2 | ID NO. |
| Phage-1 | 0.091 | 1.227 | 0.085 | 23 |
| Phage-2 | 0.072 | 2.622 | 0.125 | 159 |
| Phage-3 | 0.110 | 2.763 | 0.142 | 160 |
| Phage-4 | 0.073 | 2.739 | 0.215 | 161 |
| Phage-5 | 0.083 | 2.746 | 0.343 | 162 |
| Phage-6 | 0.067 | 2.512 | 0.167 | 163 |
| Phage-7 | 0.077 | 2.742 | 0.451 | 164 |
| Phage-8 | 0.078 | 2.729 | 0.339 | 165 |
| Phage-9 | 0.072 | 2.761 | 0.289 | 166 |
| Phage- | 0.114 | 2.798 | 0.086 | 167 |
| 10 | | | | |
| Phage- | 0.098 | 2.717 | 0.173 | 168 |
| 11 | | | | |
| Phage- | 0.097 | 2.737 | 0.221 | 169 |
| 12 | | | | |
| Phage- | 0.135 | 2.713 | 0.168 | 170 |
| 13 | | | | |
| Phage- | 0.102 | 2.737 | 0.327 | 171 |
| 14 | | | | |
| Phage- | 0.122 | 2.694 | 0.161 | 172 |
| 15 | | | | |
| Phage- | 0.089 | 2.674 | 0.186 | 173 |
| 16 | | | | |
| Phage- | 0.072 | 2.704 | 0.283 | 174 |
| 17 | | | | |
| Phage- | 0.184 | 2.820 | 0.379 | 175 |
| 18 | | | | |
| Phage- | 0.071 | 2.710 | 0.237 | 176 |
| 19 | | | | |
| Phage- | 0.082 | 2.784 | 0.344 | 177 |
| 20 | | | | |
| Phage- | 0.122 | 2.711 | 0.134 | 178 |
| 21 | | | | |
| Phage- | 0.221 | 2.717 | 0.247 | 244 |
| 22 | | | | |
| Phage- | 0.112 | 2.714 | 0.296 | 245 |
| 23 | | | | |
| Phage- | 0.081 | 2.721 | 0.258 | 246 |
| 24 | | | | |
| Phage- | 0.178 | 2.728 | 0.211 | 247 |
| 25 | | | | |
| Phage- | 0.067 | 2.814 | 0.156 | 248 |
| 26 | | | | |
| Phage- | 0.089 | 2.824 | 0.083 | 249 |
| 27 | | | | |
| Phage- | 0.142 | 2.777 | 0.134 | 250 |
| 28 | | | | |
| Phage- | 0.541 | 2.760 | 0.419 | 251 |
| 29 | | | | |
| Phage- | 0.293 | 2.748 | 0.225 | 252 |
| 30 | | | | |
| Phage- | 0.269 | 2.747 | 0.265 | 253 |
| 31 | | | | |
| Phage- | 0.219 | 2.744 | 0.143 | 254 |
| 32 | | | | |
| Phage- | 0.104 | 2.742 | 0.087 | 255 |
| 33 | | | | |
| Phage- | 0.191 | 2.740 | 0.138 | 256 |
| 34 | | | | |
| Phage- | 0.078 | 2.739 | 0.078 | 257 |
| 35 | | | | |
| Phage- | 0.239 | 2.739 | 0.194 | 258 |
| 36 | | | | |
| Phage- | 0.106 | 2.738 | 0.104 | 259 |
| 37 | | | | |
| Phage- | 0.257 | 2.738 | 0.228 | 260 |
| 38 | | | | |
| Phage- | 0.204 | 2.736 | 0.137 | 261 |
| 39 | | | | |
| Phage- | 0.317 | 2.736 | 0.166 | 262 |
| 40 | | | | |
| Phage- | 0.171 | 2.735 | 0.148 | 263 |
| 41 | | | | |
| Phage- | 0.383 | 2.735 | 0.324 | 264 |
| 42 | | | | |
| Phage- | 0.207 | 2.731 | 0.200 | 265 |
| 43 | | | | |
| Phage- | 0.183 | 2.729 | 0.144 | 266 |
| 44 | | | | |
| Phage- | 0.236 | 2.728 | 0.216 | 267 |
| 45 | | | | |
| Phage- | 0.349 | 2.728 | 0.306 | 268 |
| 46 | | | | |
| Phage- | 0.151 | 2.726 | 0.137 | 269 |
| 47 | | | | |
| Phage- | 0.269 | 2.724 | 0.111 | 270 |
| 48 | | | | |
| Phage- | 0.375 | 2.724 | 0.357 | 271 |
| 49 | | | | |
| Phage- | 0.231 | 2.723 | 0.218 | 272 |
| 50 | | | | |
| Phage- | 0.611 | 2.722 | 0.297 | 273 |
| 51 | | | | |
| Phage- | 0.159 | 2.722 | 0.136 | 274 |
| 52 | | | | |
| Phage- | 0.169 | 2.722 | 0.147 | 275 |
| 53 | | | | |
| Phage- | 0.329 | 2.721 | 0.128 | 276 |
| 54 | | | | |
| Phage- | 0.181 | 2.721 | 0.108 | 277 |
| 55 | | | | |
| Phage- | 0.297 | 2.719 | 0.169 | 278 |
| 56 | | | | |
| Phage- | 0.379 | 2.719 | 0.167 | 279 |
| 57 | | | | |
| Phage- | 2.696 | 2.719 | 2.689 | 280 |
| 58 | | | | |
| Phage- | 0.225 | 2.718 | 0.216 | 281 |
| 59 | | | | |
| Phage- | 0.321 | 2.716 | 0.319 | 282 |
| 60 | | | | |
| Phage- | 0.215 | 2.714 | 0.195 | 283 |
| 61 | | | | |
| Phage- | 0.338 | 2.713 | 0.208 | 284 |
| 62 | | | | |
| Phage- | 0.392 | 2.712 | 0.270 | 285 |
| 63 | | | | |
| Phage- | 0.223 | 2.711 | 0.168 | 286 |
| 64 | | | | |
| Phage- | 0.566 | 2.711 | 0.556 | 287 |
| 65 | | | | |
| Phage- | 0.470 | 2.711 | 0.208 | 288 |
| 66 | | | | |
| Phage- | 0.342 | 2.710 | 0.310 | 289 |
| 67 | | | | |
| Phage- | 0.267 | 2.710 | 0.133 | 290 |
| 68 | | | | |
| Phage- | 0.313 | 2.709 | 0.226 | 291 |
| 69 | | | | |
| Phage- | 0.301 | 2.706 | 0.159 | 292 |
| 70 | | | | |
| Phage- | 0.264 | 2.705 | 0.093 | 293 |
| 71 | | | | |
| Phage- | 0.444 | 2.704 | 0.418 | 294 |
| 72 | | | | |
| Phage- | 0.245 | 2.702 | 0.122 | 295 |
| 73 | | | | |
| Phage- | 0.456 | 2.702 | 0.313 | 296 |
| 74 | | | | |
| Phage- | 0.329 | 2.701 | 0.287 | 297 |
| 75 | | | | |
| Phage- | 0.337 | 2.701 | 0.214 | 298 |
| 76 | | | | |
| Phage- | 0.358 | 2.701 | 0.222 | 299 |
| 77 | | | | |
| Phage- | 0.133 | 2.699 | 0.123 | 300 |
| 78 | | | | |
| Phage- | 0.246 | 2.697 | 0.138 | 301 |
| 79 | | | | |
| Phage- | 0.220 | 2.694 | 0.196 | 302 |
| 80 | | | | |
| Phage- | 0.349 | 2.693 | 0.130 | 303 |
| 81 | | | | |
| Phage- | 0.234 | 2.691 | 0.135 | 304 |
| 82 | | | | |
| Phage- | 0.316 | 2.691 | 0.167 | 305 |
| 83 | | | | |
| Phage- | 0.324 | 2.687 | 0.202 | 306 |
| 84 | | | | |
| Phage- | 0.145 | 2.681 | 0.136 | 307 |
| 85 | | | | |
| Phage- | 0.138 | 2.674 | 0.138 | 308 |
| 86 | | | | |
| Phage- | 0.343 | 2.672 | 0.204 | 309 |
| 87 | | | | |
| Phage- | 0.185 | 2.672 | 0.082 | 310 |
| 88 | | | | |
| Phage- | 0.180 | 2.666 | 0.155 | 311 |
| 89 | | | | |
| Phage- | 0.132 | 2.664 | 0.119 | 312 |
| 90 | | | | |
| Phage- | 0.200 | 2.657 | 0.195 | 313 |
| 91 | | | | |
| Phage- | 0.247 | 2.655 | 0.149 | 314 |
| 92 | | | | |
| Phage- | 0.204 | 2.652 | 0.136 | 315 |
| 93 | | | | |
| Phage- | 0.235 | 2.631 | 0.195 | 316 |
| 94 | | | | |
| Phage- | 0.160 | 2.626 | 0.153 | 317 |
| 95 | | | | |
| Phage- | 0.178 | 2.623 | 0.160 | 318 |
| 96 | | | | |
| Phage- | 0.177 | 2.621 | 0.111 | 319 |
| 97 | | | | |
| Phage- | 0.145 | 2.594 | 0.131 | 320 |
| 98 | | | | |
| Phage- | 0.341 | 2.593 | 0.148 | 321 |
| 99 | | | | |
| Phage- | 0.347 | 2.577 | 0.171 | 322 |
| 100 | | | | |
| Phage- | 0.259 | 2.540 | 0.226 | 323 |
| 101 | | | | |
| Phage- | 0.162 | 2.538 | 0.124 | 324 |
| 102 | | | | |
| Phage- | 0.338 | 2.487 | 0.173 | 325 |
| 103 | | | | |
| Phage- | 0.087 | 2.479 | 0.074 | 326 |
| 104 | | | | |
| Phage- | 0.296 | 2.437 | 0.263 | 327 |
| 105 | | | | |
| Phage- | 0.266 | 2.413 | 0.163 | 328 |
| 106 | | | | |
| Phage- | 0.193 | 2.313 | 0.139 | 329 |
| 107 | | | | |
| Phage- | 0.140 | 2.290 | 0.128 | 330 |
| 108 | | | | |
| Phage- | 0.091 | 2.217 | 0.078 | 331 |
| 109 | | | | |
| Phage- | 0.167 | 2.036 | 0.162 | 332 |
| 110 | | | | |
| Phage- | 0.089 | 2.029 | 0.083 | 333 |
| 111 | | | | |
| Phage- | 0.372 | 2.011 | 0.154 | 334 |
| 112 | | | | |
| Phage- | 0.290 | 2.001 | 0.266 | 335 |
| 113 | | | | |
| Phage- | 0.191 | 1.935 | 0.143 | 336 |
| 114 | | | | |
| Phage- | 0.215 | 1.805 | 0.172 | 337 |
| 115 | | | | |
| Phage- | 0.234 | 1.730 | 0.120 | 338 |
| 116 | | | | |
| Phage- | 0.174 | 1.648 | 0.110 | 339 |
| 117 | | | | |
| Phage- | 0.124 | 1.475 | 0.118 | 340 |
| 118 | | | | |
| Phage- | 0.203 | 1.442 | 0.070 | 341 |
| 119 | | | | |
| Phage- | 0.183 | 1.441 | 0.134 | 342 |
| 120 | | | | |
| Phage- | 0.150 | 1.332 | 0.132 | 343 |
| 121 | | | | |
| Phage- | 0.150 | 0.814 | 0.148 | 344 |
| 122 | | | | |
| Phage- | 0.107 | 0.698 | 0.099 | 345 |
| 123 | | | | |
| Phage- | 0.084 | 0.636 | 0.078 | 346 |
| 124 | | | | |
| Phage- | 0.182 | 0.508 | 0.064 | 347 |
| 125 | | | | |
| Phage- | 0.185 | 0.488 | 0.131 | 348 |
| 126 | | | | |
| Phage- | 0.110 | 0.480 | 0.108 | 349 |
| 127 | | | | |
| Phage- | 0.196 | 0.302 | 0.153 | 350 |
| 128 | | | | |
| Phage- | 0.126 | 0.194 | 0.095 | 351 |
| 129 | | | | |
| Phage- | 0.146 | 0.143 | 0.166 | 352 |
| 130 | | | | |
| Phage- | 0.166 | 0.105 | 2.655 | 353 |
| 131 | | | | |
| Phage- | 0.268 | 0.099 | 2.674 | 354 |
| 132 | | | | |
| Phage- | 0.117 | 0.098 | 0.125 | 355 |
| 133 | | | | |
| Phage- | 0.113 | 0.095 | 0.134 | 356 |
| 134 | | | | |
| Phage- | 0.234 | 0.090 | 2.682 | 357 |
| 135 | | | | |
| Phage- | 0.113 | 0.089 | 2.695 | 358 |
| 136 | | | | |
| Phage- | 0.097 | 0.088 | 0.099 | 359 |
| 137 | | | | |
| Phage- | 0.082 | 0.088 | 0.073 | 360 |
| 138 | | | | |
| Phage- | 0.087 | 0.085 | 0.095 | 361 |
| 139 | | | | |
| Phage- | 0.087 | 0.084 | 2.843 | 362 |
| 140 | | | | |
| Phage- | 0.094 | 0.083 | 0.100 | 363 |
| 141 | | | | |
| Phage- | 0.121 | 0.082 | 0.133 | 364 |
| 142 | | | | |
| Phage- | 0.087 | 0.078 | 0.138 | 365 |
| 143 | | | | |
| Phage- | 0.092 | 0.077 | 0.098 | 366 |
| 144 | | | | |
| Phage- | 0.083 | 0.077 | 0.092 | 367 |
| 145 | | | | |
| Phage- | 0.090 | 0.076 | 0.111 | 368 |
| 146 | | | | |
| Phage- | 0.098 | 0.076 | 0.101 | 369 |
| 147 | | | | |
| Phage- | 0.090 | 0.076 | 0.109 | 370 |
| 148 | | | | |
| Phage- | 0.070 | 0.075 | 0.069 | 371 |
| 149 | | | | |
| Phage- | 0.079 | 0.075 | 0.088 | 372 |
| 150 | | | | |
| Phage- | 0.081 | 0.075 | 0.112 | 373 |
| 151 | | | | |
| Phage- | 0.083 | 0.075 | 0.092 | 374 |
| 152 | | | | |
| Phage- | 0.138 | 0.073 | 0.252 | 375 |
| 153 | | | | |
| Phage- | 0.096 | 0.073 | 0.098 | 376 |
| 154 | | | | |
| Phage- | 0.119 | 0.072 | 0.280 | 377 |
| 155 | | | | |
| Phage- | 0.074 | 0.072 | 0.149 | 378 |
| 156 | | | | |
| Phage- | 0.078 | 0.072 | 0.113 | 379 |
| 157 | | | | |
| Phage- | 0.081 | 0.071 | 0.136 | 380 |
| 158 | | | | |
| Phage- | 0.077 | 0.070 | 0.087 | 381 |
| 159 | | | | |
| Phage- | 0.089 | 0.070 | 0.110 | 382 |
| 160 | | | | |
| Phage- | 0.085 | 0.070 | 0.107 | 383 |
| 161 | | | | |
| Phage- | 0.083 | 0.069 | 0.191 | 384 |
| 162 | | | | |
| Phage- | 0.085 | 0.069 | 0.089 | 385 |
| 163 | | | | |
| Phage- | 0.094 | 0.068 | 0.124 | 386 |
| 164 | | | | |
| Phage- | 0.084 | 0.068 | 2.688 | 387 |
| 165 | | | | |
| Phage- | 0.229 | 0.067 | 2.668 | 388 |
| 166 | | | | |
| Phage- | 0.070 | 0.067 | 0.389 | 389 |
| 167 | | | | |
| Phage- | 0.073 | 0.066 | 0.089 | 390 |
| 168 | | | | |
| Phage- | 0.084 | 0.065 | 0.130 | 391 |
| 169 | | | | |
| Phage- | 0.082 | 0.064 | 0.150 | 392 |
| 170 | | | | |
| Phage- | 0.065 | 0.064 | 0.212 | 393 |
| 171 | | | | |
| Phage- | 0.141 | 0.063 | 0.371 | 394 |
| 172 | | | | |
| Phage- | 0.096 | 0.062 | 0.110 | 395 |
| 173 | | | | |
| Phage- | 0.072 | 0.062 | 0.086 | 396 |
| 174 | | | | |
| Phage- | 0.185 | 0.062 | 0.251 | 397 |
| 175 | | | | |
| Phage- | 0.167 | 0.061 | 0.308 | 398 |
| 176 | | | | |
| Phage- | 0.066 | 0.061 | 0.181 | 399 |
| 177 | | | | |
| Phage- | 0.186 | 0.061 | 0.403 | 400 |
| 178 | | | | |
| Phage- | 0.067 | 0.061 | 0.149 | 401 |
| 179 | | | | |
| Phage- | 0.074 | 0.057 | 0.082 | 402 |
| 180 | | | | |
| Phage- | 0.169 | 2.685 | 0.169 | 403 |
| 181 | | | | |
| Phage- | 0.096 | 2.715 | 0.097 | 404 |
| 182 | | | | |
| Phage- | 0.110 | 2.671 | 0.111 | 405 |
| 183 | | | | |
| Phage- | 0.150 | 2.408 | 0.151 | 406 |
| 184 | | | | |
| Phage- | 0.150 | 2.515 | 0.151 | 407 |
| 185 | | | | |
| Phage- | 0.234 | 2.711 | 0.236 | 408 |
| 186 | | | | |
| Phage- | 0.379 | 2.712 | 0.381 | 409 |
| 187 | | | | |
| Phage- | 0.131 | 2.710 | 0.134 | 410 |
| 188 | | | | |
| Phage- | 0.093 | 2.697 | 0.097 | 411 |
| 189 | | | | |
| Phage- | 0.146 | 2.601 | 0.150 | 412 |
| 190 | | | | |
| Phage- | 0.144 | 2.565 | 0.149 | 413 |
| 191 | | | | |
| Phage- | 0.170 | 2.513 | 0.175 | 414 |
| 192 | | | | |
| Phage- | 0.212 | 2.706 | 0.218 | 415 |
| 193 | | | | |
| Phage- | 0.118 | 2.054 | 0.124 | 416 |
| 194 | | | | |
| Phage- | 0.202 | 2.626 | 0.209 | 417 |
| 195 | | | | |
| Phage- | 0.104 | 2.786 | 0.113 | 418 |
| 196 | | | | |
| Phage- | 0.148 | 2.745 | 0.157 | 419 |
| 197 | | | | |
| Phage- | 0.182 | 2.701 | 0.191 | 420 |
| 198 | | | | |
| Phage- | 0.076 | 1.157 | 0.079 | 421 |
| 199 | | | | |
| Phage- | 0.080 | 2.690 | 0.089 | 422 |
| 200 | | | | |
| Phage- | 0.142 | 1.423 | 0.147 | 423 |
| 201 | | | | |
| Phage- | 0.086 | 2.755 | 0.098 | 424 |
| 202 | | | | |
| Phage- | 0.062 | 2.685 | 0.075 | 425 |
| 203 | | | | |
| Phage- | 0.070 | 2.639 | 0.083 | 426 |
| 204 | | | | |
| Phage- | 0.163 | 2.636 | 0.175 | 427 |
| 205 | | | | |
| Phage- | 0.088 | 2.686 | 0.102 | 428 |
| 206 | | | | |
| Phage- | 0.076 | 2.729 | 0.090 | 429 |
| 207 | | | | |
| Phage- | 0.093 | 2.754 | 0.109 | 430 |
| 208 | | | | |
| Phage- | 0.099 | 2.013 | 0.110 | 431 |
| 209 | | | | |
| Phage- | 0.144 | 2.664 | 0.159 | 432 |
| 210 | | | | |
| Phage- | 0.167 | 2.511 | 0.182 | 433 |
| 211 | | | | |
| Phage- | 0.104 | 2.774 | 0.121 | 434 |
| 212 | | | | |
| Phage- | 0.132 | 2.746 | 0.148 | 435 |
| 213 | | | | |
| Phage- | 0.095 | 2.597 | 0.112 | 436 |
| 214 | | | | |
| Phage- | 0.252 | 2.703 | 0.269 | 437 |
| 215 | | | | |
| Phage- | 0.091 | 2.822 | 0.110 | 438 |
| 216 | | | | |
| Phage- | 0.204 | 2.710 | 0.222 | 439 |
| 217 | | | | |
| Phage- | 0.210 | 2.711 | 0.228 | 440 |
| 218 | | | | |
| Phage- | 0.108 | 2.666 | 0.128 | 441 |
| 219 | | | | |
| Phage- | 0.150 | 2.461 | 0.168 | 442 |
| 220 | | | | |
| Phage- | 0.130 | 2.710 | 0.152 | 443 |
| 221 | | | | |
| Phage- | 0.077 | 1.498 | 0.089 | 444 |
| 222 | | | | |
| Phage- | 0.104 | 2.740 | 0.128 | 445 |
| 223 | | | | |
| Phage- | 0.087 | 2.805 | 0.112 | 446 |
| 224 | | | | |
| Phage- | 0.073 | 0.666 | 0.078 | 447 |
| 225 | | | | |
| Phage- | 0.096 | 2.436 | 0.118 | 448 |
| 226 | | | | |
| Phage- | 0.117 | 2.752 | 0.141 | 449 |
| 227 | | | | |
| Phage- | 0.067 | 2.324 | 0.088 | 450 |
| 228 | | | | |
| Phage- | 0.104 | 2.050 | 0.123 | 451 |
| 229 | | | | |
| Phage- | 0.154 | 2.810 | 0.180 | 452 |
| 230 | | | | |
| Phage- | 0.078 | 2.368 | 0.101 | 453 |
| 231 | | | | |
| Phage- | 0.075 | 1.658 | 0.091 | 454 |
| 232 | | | | |
| Phage- | 0.176 | 2.738 | 0.203 | 455 |
| 233 | | | | |
| Phage- | 0.072 | 2.693 | 0.099 | 456 |
| 234 | | | | |
| Phage- | 0.125 | 2.662 | 0.151 | 457 |
| 235 | | | | |
| Phage- | 0.073 | 2.358 | 0.097 | 458 |
| 236 | | | | |
| Phage- | 0.082 | 2.397 | 0.107 | 459 |
| 237 | | | | |
| Phage- | 0.070 | 2.653 | 0.099 | 460 |
| 238 | | | | |
| Phage- | 0.308 | 2.740 | 0.335 | 461 |
| 239 | | | | |
| Phage- | 0.138 | 1.860 | 0.157 | 462 |
| 240 | | | | |
| Phage- | 0.123 | 2.555 | 0.150 | 463 |
| 241 | | | | |
| Phage- | 0.076 | 2.684 | 0.106 | 464 |
| 242 | | | | |
| Phage- | 0.108 | 2.754 | 0.138 | 465 |
| 243 | | | | |
| Phage- | 0.193 | 2.682 | 0.221 | 466 |
| 244 | | | | |
| Phage- | 0.069 | 2.800 | 0.100 | 467 |
| 245 | | | | |
| Phage- | 0.113 | 2.714 | 0.143 | 468 |
| 246 | | | | |
| Phage- | 0.074 | 2.545 | 0.104 | 469 |
| 247 | | | | |
| Phage- | 0.093 | 2.604 | 0.124 | 470 |
| 248 | | | | |
| Phage- | 0.067 | 1.762 | 0.088 | 471 |
| 249 | | | | |
| Phage- | 0.065 | 2.686 | 0.098 | 472 |
| 250 | | | | |
| Phage- | 0.139 | 2.622 | 0.172 | 473 |
| 251 | | | | |
| Phage- | 0.109 | 2.726 | 0.144 | 474 |
| 252 | | | | |
| Phage- | 0.064 | 2.613 | 0.098 | 475 |
| 253 | | | | |
| Phage- | 0.099 | 2.741 | 0.136 | 476 |
| 254 | | | | |
| Phage- | 0.112 | 2.752 | 0.148 | 477 |
| 255 | | | | |
| Phage- | 0.162 | 2.736 | 0.198 | 478 |
| 256 | | | | |
| Phage- | 0.079 | 2.728 | 0.116 | 479 |
| 257 | | | | |
| Phage- | 0.084 | 2.748 | 0.121 | 480 |
| 258 | | | | |
| Phage- | 0.096 | 2.728 | 0.133 | 481 |
| 259 | | | | |
| Phage- | 0.155 | 2.705 | 0.191 | 482 |
| 260 | | | | |
| Phage- | 0.092 | 1.982 | 0.120 | 483 |
| 261 | | | | |
| Phage- | 0.090 | 2.775 | 0.131 | 484 |
| 262 | | | | |
| Phage- | 0.230 | 2.723 | 0.268 | 485 |
| 263 | | | | |
| Phage- | 0.088 | 2.711 | 0.128 | 486 |
| 264 | | | | |
| Phage- | 0.075 | 2.310 | 0.109 | 487 |
| 265 | | | | |
| Phage- | 0.137 | 2.694 | 0.177 | 488 |
| 266 | | | | |
| Phage- | 0.084 | 2.838 | 0.126 | 489 |
| 267 | | | | |
| Phage- | 0.090 | 2.491 | 0.127 | 490 |
| 268 | | | | |
| Phage- | 0.069 | 2.623 | 0.110 | 491 |
| 269 | | | | |
| Phage- | 0.087 | 2.876 | 0.131 | 492 |
| 270 | | | | |
| Phage- | 0.091 | 2.720 | 0.134 | 493 |
| 271 | | | | |
| Phage- | 0.091 | 2.761 | 0.135 | 494 |
| 272 | | | | |
| Phage- | 0.076 | 2.753 | 0.120 | 495 |
| 273 | | | | |
| Phage- | 0.085 | 2.789 | 0.129 | 496 |
| 274 | | | | |
| Phage- | 0.233 | 2.697 | 0.274 | 497 |
| 275 | | | | |
| Phage- | 0.091 | 2.829 | 0.137 | 498 |
| 276 | | | | |
| Phage- | 0.155 | 2.612 | 0.197 | 499 |
| 277 | | | | |
| Phage- | 0.165 | 2.656 | 0.208 | 500 |
| 278 | | | | |
| Phage- | 0.091 | 2.666 | 0.135 | 501 |
| 279 | | | | |
| Phage- | 0.091 | 2.728 | 0.137 | 502 |
| 280 | | | | |
| Phage- | 0.121 | 2.687 | 0.165 | 503 |
| 281 | | | | |
| Phage- | 0.170 | 2.409 | 0.209 | 504 |
| 282 | | | | |
| Phage- | 0.329 | 2.719 | 0.371 | 505 |
| 283 | | | | |
| Phage- | 0.099 | 2.722 | 0.145 | 506 |
| 284 | | | | |
| Phage- | 0.393 | 2.723 | 0.434 | 507 |
| 285 | | | | |
| Phage- | 0.103 | 2.637 | 0.148 | 508 |
| 286 | | | | |
| Phage- | 0.084 | 2.710 | 0.131 | 509 |
| 287 | | | | |
| Phage- | 0.068 | 2.707 | 0.116 | 510 |
| 288 | | | | |
| Phage- | 0.087 | 2.710 | 0.135 | 511 |
| 289 | | | | |
| Phage- | 0.266 | 1.816 | 0.294 | 512 |
| 290 | | | | |
| Phage- | 0.138 | 2.736 | 0.186 | 513 |
| 291 | | | | |
| Phage- | 0.085 | 2.778 | 0.135 | 514 |
| 292 | | | | |
| Phage- | 0.137 | 2.698 | 0.184 | 515 |
| 293 | | | | |
| Phage- | 0.153 | 2.748 | 0.202 | 516 |
| 294 | | | | |
| Phage- | 0.111 | 1.025 | 0.129 | 517 |
| 295 | | | | |
| Phage- | 0.067 | 1.614 | 0.097 | 518 |
| 296 | | | | |
| Phage- | 0.130 | 2.740 | 0.180 | 519 |
| 297 | | | | |
| Phage- | 0.151 | 0.742 | 0.162 | 520 |
| 298 | | | | |
| Phage- | 0.090 | 2.747 | 0.142 | 521 |
| 299 | | | | |
| Phage- | 0.120 | 2.716 | 0.171 | 522 |
| 300 | | | | |
| Phage- | 0.084 | 2.756 | 0.136 | 523 |
| 301 | | | | |
| Phage- | 0.078 | 2.596 | 0.128 | 524 |
| 302 | | | | |
| Phage- | 0.220 | 2.754 | 0.270 | 525 |
| 303 | | | | |
| Phage- | 0.091 | 2.810 | 0.145 | 526 |
| 304 | | | | |
| Phage- | 0.119 | 2.341 | 0.163 | 527 |
| 305 | | | | |
| Phage- | 0.075 | 2.759 | 0.129 | 528 |
| 306 | | | | |
| Phage- | 0.064 | 2.052 | 0.105 | 529 |
| 307 | | | | |
| Phage- | 0.123 | 2.715 | 0.176 | 530 |
| 308 | | | | |
| Phage- | 0.138 | 2.750 | 0.192 | 531 |
| 309 | | | | |
| Phage- | 0.096 | 2.315 | 0.141 | 532 |
| 310 | | | | |
| Phage- | 0.273 | 2.656 | 0.323 | 533 |
| 311 | | | | |
| Phage- | 0.077 | 2.730 | 0.134 | 534 |
| 312 | | | | |
| Phage- | 0.064 | 0.214 | 0.068 | 535 |
| 313 | | | | |
| Phage- | 0.095 | 2.798 | 0.154 | 536 |
| 314 | | | | |
| Phage- | 0.068 | 0.455 | 0.077 | 537 |
| 315 | | | | |
| Phage- | 0.190 | 2.722 | 0.246 | 538 |
| 316 | | | | |
| Phage- | 0.172 | 2.722 | 0.229 | 539 |
| 317 | | | | |
| Phage- | 0.082 | 2.707 | 0.141 | 540 |
| 318 | | | | |
| Phage- | 0.066 | 2.001 | 0.110 | 541 |
| 319 | | | | |
| Phage- | 0.094 | 2.815 | 0.156 | 542 |
| 320 | | | | |
| Phage- | 0.099 | 2.694 | 0.159 | 543 |
| 321 | | | | |
| Phage- | 0.105 | 2.431 | 0.159 | 544 |
| 322 | | | | |
| Phage- | 0.090 | 2.753 | 0.153 | 545 |
| 323 | | | | |
| Phage- | 0.246 | 2.579 | 0.300 | 546 |
| 324 | | | | |
| Phage- | 0.072 | 2.701 | 0.134 | 547 |
| 325 | | | | |
| Phage- | 0.097 | 2.817 | 0.161 | 548 |
| 326 | | | | |
| Phage- | 0.087 | 2.167 | 0.136 | 549 |
| 327 | | | | |
| Phage- | 0.086 | 2.748 | 0.149 | 550 |
| 328 | | | | |
| Phage- | 0.195 | 2.709 | 0.254 | 551 |
| 329 | | | | |
| Phage- | 0.094 | 2.826 | 0.159 | 552 |
| 330 | | | | |
| Phage- | 0.076 | 2.453 | 0.134 | 553 |
| 331 | | | | |
| Phage- | 0.102 | 2.802 | 0.168 | 554 |
| 332 | | | | |
| Phage- | 0.092 | 2.854 | 0.160 | 555 |
| 333 | | | | |
| Phage- | 0.064 | 2.777 | 0.131 | 556 |
| 334 | | | | |
| Phage- | 0.110 | 2.725 | 0.175 | 557 |
| 335 | | | | |
| Phage- | 0.098 | 0.770 | 0.115 | 558 |
| 336 | | | | |
| Phage- | 0.324 | 2.701 | 0.384 | 559 |
| 337 | | | | |
| Phage- | 0.122 | 2.748 | 0.189 | 560 |
| 338 | | | | |
| Phage- | 0.110 | 2.734 | 0.177 | 561 |
| 339 | | | | |
| Phage- | 0.081 | 2.831 | 0.152 | 562 |
| 340 | | | | |
| Phage- | 0.076 | 2.320 | 0.133 | 563 |
| 341 | | | | |
| Phage- | 0.072 | 2.689 | 0.141 | 564 |
| 342 | | | | |
| Phage- | 0.089 | 2.810 | 0.162 | 565 |
| 343 | | | | |
| Phage- | 0.078 | 2.745 | 0.149 | 566 |
| 344 | | | | |
| Phage- | 0.116 | 2.710 | 0.186 | 567 |
| 345 | | | | |
| Phage- | 0.073 | 1.637 | 0.115 | 568 |
| 346 | | | | |
| Phage- | 0.104 | 2.723 | 0.175 | 569 |
| 347 | | | | |
| Phage- | 0.125 | 2.705 | 0.196 | 570 |
| 348 | | | | |
| Phage- | 0.185 | 2.664 | 0.254 | 571 |
| 349 | | | | |
| Phage- | 0.071 | 2.089 | 0.127 | 572 |
| 350 | | | | |
| Phage- | 0.138 | 2.739 | 0.211 | 573 |
| 351 | | | | |
| Phage- | 0.090 | 2.580 | 0.160 | 574 |
| 352 | | | | |
| Phage- | 0.075 | 2.718 | 0.150 | 575 |
| 353 | | | | |
| Phage- | 0.091 | 2.672 | 0.165 | 576 |
| 354 | | | | |
| Phage- | 0.080 | 1.244 | 0.113 | 577 |
| 355 | | | | |
| Phage- | 0.109 | 1.678 | 0.154 | 578 |
| 356 | | | | |
| Phage- | 0.071 | 2.746 | 0.148 | 579 |
| 357 | | | | |
| Phage- | 0.086 | 2.742 | 0.163 | 580 |
| 358 | | | | |
| Phage- | 0.173 | 2.720 | 0.247 | 581 |
| 359 | | | | |
| Phage- | 0.088 | 1.633 | 0.133 | 582 |
| 360 | | | | |
| Phage- | 0.101 | 2.723 | 0.178 | 583 |
| 361 | | | | |
| Phage- | 0.164 | 2.657 | 0.238 | 584 |
| 362 | | | | |
| Phage- | 0.203 | 2.362 | 0.268 | 585 |
| 363 | | | | |
| Phage- | 0.079 | 2.745 | 0.159 | 586 |
| 364 | | | | |
| Phage- | 0.083 | 2.459 | 0.155 | 587 |
| 365 | | | | |
| Phage- | 0.073 | 2.727 | 0.153 | 588 |
| 366 | | | | |
| Phage- | 0.067 | 2.814 | 0.151 | 589 |
| 367 | | | | |
| Phage- | 0.083 | 0.963 | 0.110 | 590 |
| 368 | | | | |
| Phage- | 0.081 | 2.380 | 0.153 | 591 |
| 369 | | | | |
| Phage- | 0.102 | 2.736 | 0.185 | 592 |
| 370 | | | | |
| Phage- | 0.080 | 2.832 | 0.167 | 593 |
| 371 | | | | |
| Phage- | 0.065 | 2.748 | 0.150 | 594 |
| 372 | | | | |
| Phage- | 0.075 | 2.625 | 0.157 | 595 |
| 373 | | | | |
| Phage- | 0.103 | 2.682 | 0.186 | 596 |
| 374 | | | | |
| Phage- | 0.293 | 2.501 | 0.364 | 597 |
| 375 | | | | |
| Phage- | 0.098 | 2.737 | 0.184 | 598 |
| 376 | | | | |
| Phage- | 0.101 | 2.645 | 0.185 | 599 |
| 377 | | | | |
| Phage- | 0.145 | 2.669 | 0.229 | 600 |
| 378 | | | | |
| Phage- | 0.270 | 2.714 | 0.352 | 601 |
| 379 | | | | |
| Phage- | 0.085 | 2.836 | 0.178 | 602 |
| 380 | | | | |
| Phage- | 0.171 | 2.722 | 0.257 | 603 |
| 381 | | | | |
| Phage- | 0.102 | 2.734 | 0.191 | 604 |
| 382 | | | | |
| Phage- | 0.066 | 2.054 | 0.135 | 605 |
| 383 | | | | |
| Phage- | 0.083 | 2.420 | 0.164 | 606 |
| 384 | | | | |
| Phage- | 0.184 | 2.744 | 0.273 | 607 |
| 385 | | | | |
| Phage- | 0.067 | 2.569 | 0.153 | 608 |
| 386 | | | | |
| Phage- | 0.065 | 2.744 | 0.158 | 609 |
| 387 | | | | |
| Phage- | 0.114 | 2.472 | 0.196 | 610 |
| 388 | | | | |
| Phage- | 0.107 | 2.269 | 0.182 | 611 |
| 389 | | | | |
| Phage- | 0.112 | 2.686 | 0.202 | 612 |
| 390 | | | | |
| Phage- | 0.138 | 2.736 | 0.229 | 613 |
| 391 | | | | |
| Phage- | 0.093 | 2.368 | 0.173 | 614 |
| 392 | | | | |
| Phage- | 0.483 | 2.699 | 0.562 | 615 |
| 393 | | | | |
| Phage- | 0.084 | 2.532 | 0.172 | 616 |
| 394 | | | | |
| Phage- | 0.117 | 2.755 | 0.212 | 617 |
| 395 | | | | |
| Phage- | 0.097 | 2.816 | 0.195 | 618 |
| 396 | | | | |
| Phage- | 0.151 | 2.741 | 0.244 | 619 |
| 397 | | | | |
| Phage- | 0.104 | 2.325 | 0.185 | 620 |
| 398 | | | | |
| Phage- | 0.293 | 2.705 | 0.381 | 621 |
| 399 | | | | |
| Phage- | 0.193 | 2.756 | 0.286 | 622 |
| 400 | | | | |
| Phage- | 0.072 | 2.725 | 0.169 | 623 |
| 401 | | | | |
| Phage- | 0.186 | 2.669 | 0.278 | 624 |
| 402 | | | | |
| Phage- | 0.070 | 2.764 | 0.170 | 625 |
| 403 | | | | |
| Phage- | 0.088 | 2.759 | 0.188 | 626 |
| 404 | | | | |
| Phage- | 0.077 | 2.692 | 0.176 | 627 |
| 405 | | | | |
| Phage- | 0.094 | 2.732 | 0.193 | 628 |
| 406 | | | | |
| Phage- | 0.126 | 2.695 | 0.223 | 629 |
| 407 | | | | |
| Phage- | 0.173 | 2.708 | 0.269 | 630 |
| 408 | | | | |
| Phage- | 0.158 | 2.706 | 0.255 | 631 |
| 409 | | | | |
| Phage- | 0.118 | 2.686 | 0.216 | 632 |
| 410 | | | | |
| Phage- | 0.126 | 2.757 | 0.227 | 633 |
| 411 | | | | |
| Phage- | 0.074 | 2.841 | 0.181 | 634 |
| 412 | | | | |
| Phage- | 0.075 | 1.993 | 0.150 | 635 |
| 413 | | | | |
| Phage- | 0.099 | 2.766 | 0.204 | 636 |
| 414 | | | | |
| Phage- | 0.145 | 2.713 | 0.246 | 637 |
| 415 | | | | |
| Phage- | 0.066 | 2.681 | 0.169 | 638 |
| 416 | | | | |
| Phage- | 0.296 | 2.734 | 0.393 | 639 |
| 417 | | | | |
| Phage- | 0.073 | 2.755 | 0.179 | 640 |
| 418 | | | | |
| Phage- | 0.088 | 1.788 | 0.156 | 641 |
| 419 | | | | |
| Phage- | 0.146 | 2.754 | 0.251 | 642 |
| 420 | | | | |
| Phage- | 0.075 | 2.740 | 0.183 | 643 |
| 421 | | | | |
| Phage- | 0.209 | 2.722 | 0.311 | 644 |
| 422 | | | | |
| Phage- | 0.072 | 2.741 | 0.180 | 645 |
| 423 | | | | |
| Phage- | 0.096 | 2.724 | 0.203 | 646 |
| 424 | | | | |
| Phage- | 0.099 | 2.822 | 0.212 | 647 |
| 425 | | | | |
| Phage- | 0.110 | 2.746 | 0.219 | 648 |
| 426 | | | | |
| Phage- | 0.112 | 2.738 | 0.221 | 649 |
| 427 | | | | |
| Phage- | 0.155 | 2.722 | 0.262 | 650 |
| 428 | | | | |
| Phage- | 0.096 | 2.723 | 0.206 | 651 |
| 429 | | | | |
| Phage- | 0.102 | 1.575 | 0.164 | 652 |
| 430 | | | | |
| Phage- | 0.079 | 2.715 | 0.190 | 653 |
| 431 | | | | |
| Phage- | 0.123 | 2.737 | 0.235 | 654 |
| 432 | | | | |
| Phage- | 0.125 | 2.754 | 0.238 | 655 |
| 433 | | | | |
| Phage- | 0.082 | 2.832 | 0.201 | 656 |
| 434 | | | | |
| Phage- | 0.138 | 2.728 | 0.251 | 657 |
| 435 | | | | |
| Phage- | 0.156 | 2.761 | 0.269 | 658 |
| 436 | | | | |
| Phage- | 0.104 | 1.959 | 0.185 | 659 |
| 437 | | | | |
| Phage- | 0.062 | 2.736 | 0.178 | 660 |
| 438 | | | | |
| Phage- | 0.090 | 2.455 | 0.194 | 661 |
| 439 | | | | |
| Phage- | 0.081 | 2.815 | 0.203 | 662 |
| 440 | | | | |
| Phage- | 0.077 | 2.787 | 0.198 | 663 |
| 441 | | | | |
| Phage- | 0.080 | 2.819 | 0.203 | 664 |
| 442 | | | | |
| Phage- | 0.107 | 2.733 | 0.225 | 665 |
| 443 | | | | |
| Phage- | 0.151 | 2.713 | 0.268 | 666 |
| 444 | | | | |
| Phage- | 0.068 | 2.463 | 0.177 | 667 |
| 445 | | | | |
| Phage- | 0.106 | 2.810 | 0.231 | 668 |
| 446 | | | | |
| Phage- | 0.071 | 2.749 | 0.195 | 669 |
| 447 | | | | |
| Phage- | 0.105 | 2.758 | 0.228 | 670 |
| 448 | | | | |
| Phage- | 0.073 | 1.323 | 0.133 | 671 |
| 449 | | | | |
| Phage- | 0.084 | 2.714 | 0.210 | 672 |
| 450 | | | | |
| Phage- | 0.184 | 2.670 | 0.303 | 673 |
| 451 | | | | |
| Phage- | 0.092 | 2.510 | 0.208 | 674 |
| 452 | | | | |
| Phage- | 0.073 | 1.776 | 0.155 | 675 |
| 453 | | | | |
| Phage- | 0.080 | 2.731 | 0.208 | 676 |
| 454 | | | | |
| Phage- | 0.082 | 2.716 | 0.210 | 677 |
| 455 | | | | |
| Phage- | 0.102 | 2.679 | 0.227 | 678 |
| 456 | | | | |
| Phage- | 0.090 | 2.836 | 0.224 | 679 |
| 457 | | | | |
| Phage- | 0.072 | 2.839 | 0.207 | 680 |
| 458 | | | | |
| Phage- | 0.097 | 2.688 | 0.224 | 681 |
| 459 | | | | |
| Phage- | 0.195 | 2.718 | 0.319 | 682 |
| 460 | | | | |
| Phage- | 0.077 | 1.592 | 0.151 | 683 |
| 461 | | | | |
| Phage- | 0.170 | 2.726 | 0.297 | 684 |
| 462 | | | | |
| Phage- | 0.214 | 2.745 | 0.340 | 685 |
| 463 | | | | |
| Phage- | 0.157 | 2.675 | 0.283 | 686 |
| 464 | | | | |
| Phage- | 0.069 | 2.061 | 0.169 | 687 |
| 465 | | | | |
| Phage- | 0.076 | 1.515 | 0.148 | 688 |
| 466 | | | | |
| Phage- | 0.081 | 2.743 | 0.215 | 689 |
| 467 | | | | |
| Phage- | 0.084 | 2.716 | 0.217 | 690 |
| 468 | | | | |
| Phage- | 0.147 | 2.552 | 0.268 | 691 |
| 469 | | | | |
| Phage- | 0.082 | 2.846 | 0.222 | 692 |
| 470 | | | | |
| Phage- | 0.086 | 2.805 | 0.226 | 693 |
| 471 | | | | |
| Phage- | 0.175 | 0.609 | 0.197 | 694 |
| 472 | | | | |
| Phage- | 0.075 | 2.620 | 0.208 | 695 |
| 473 | | | | |
| Phage- | 0.126 | 2.828 | 0.268 | 696 |
| 474 | | | | |
| Phage- | 0.312 | 2.705 | 0.438 | 697 |
| 475 | | | | |
| Phage- | 0.400 | 2.713 | 0.523 | 698 |
| 476 | | | | |
| Phage- | 0.086 | 2.736 | 0.228 | 699 |
| 477 | | | | |
| Phage- | 0.529 | 2.727 | 0.647 | 700 |
| 478 | | | | |
| Phage- | 0.138 | 2.721 | 0.277 | 701 |
| 479 | | | | |
| Phage- | 0.069 | 2.734 | 0.214 | 702 |
| 480 | | | | |
| Phage- | 0.151 | 2.738 | 0.292 | 703 |
| 481 | | | | |
| Phage- | 0.091 | 2.537 | 0.225 | 704 |
| 482 | | | | |
| Phage- | 0.144 | 1.356 | 0.211 | 705 |
| 483 | | | | |
| Phage- | 0.090 | 2.729 | 0.237 | 706 |
| 484 | | | | |
| Phage- | 0.222 | 2.736 | 0.362 | 707 |
| 485 | | | | |
| Phage- | 0.104 | 2.744 | 0.252 | 708 |
| 486 | | | | |
| Phage- | 0.126 | 2.229 | 0.244 | 709 |
| 487 | | | | |
| Phage- | 0.111 | 2.829 | 0.264 | 710 |
| 488 | | | | |
| Phage- | 0.161 | 2.729 | 0.306 | 711 |
| 489 | | | | |
| Phage- | 0.083 | 2.307 | 0.209 | 712 |
| 490 | | | | |
| Phage- | 0.243 | 2.802 | 0.388 | 713 |
| 491 | | | | |
| Phage- | 0.075 | 2.690 | 0.224 | 714 |
| 492 | | | | |
| Phage- | 0.118 | 2.571 | 0.260 | 715 |
| 493 | | | | |
| Phage- | 0.087 | 1.584 | 0.174 | 716 |
| 494 | | | | |
| Phage- | 0.188 | 2.717 | 0.335 | 717 |
| 495 | | | | |
| Phage- | 0.080 | 2.715 | 0.233 | 718 |
| 496 | | | | |
| Phage- | 0.078 | 2.845 | 0.238 | 719 |
| 497 | | | | |
| Phage- | 0.079 | 2.822 | 0.240 | 720 |
| 498 | | | | |
| Phage- | 0.170 | 1.697 | 0.260 | 721 |
| 499 | | | | |
| Phage- | 0.169 | 2.658 | 0.317 | 722 |
| 500 | | | | |
| Phage- | 0.069 | 2.028 | 0.186 | 723 |
| 501 | | | | |
| Phage- | 0.125 | 2.719 | 0.281 | 724 |
| 502 | | | | |
| Phage- | 0.220 | 2.657 | 0.367 | 725 |
| 503 | | | | |
| Phage- | 0.099 | 2.756 | 0.259 | 726 |
| 504 | | | | |
| Phage- | 0.100 | 0.083 | 0.099 | 727 |
| 505 | | | | |
| Phage- | 0.089 | 2.832 | 0.256 | 728 |
| 506 | | | | |
| Phage- | 0.242 | 2.757 | 0.396 | 729 |
| 507 | | | | |
| Phage- | 0.079 | 2.711 | 0.240 | 730 |
| 508 | | | | |
| Phage- | 0.162 | 2.698 | 0.318 | 731 |
| 509 | | | | |
| Phage- | 0.106 | 2.734 | 0.268 | 732 |
| 510 | | | | |
| Phage- | 0.082 | 2.772 | 0.249 | 733 |
| 511 | | | | |
| Phage- | 0.081 | 2.668 | 0.241 | 734 |
| 512 | | | | |
| Phage- | 0.118 | 2.745 | 0.282 | 735 |
| 513 | | | | |
| Phage- | 0.129 | 2.725 | 0.292 | 736 |
| 514 | | | | |
| Phage- | 0.093 | 2.834 | 0.265 | 737 |
| 515 | | | | |
| Phage- | 0.101 | 2.714 | 0.267 | 738 |
| 516 | | | | |
| Phage- | 0.080 | 2.362 | 0.225 | 739 |
| 517 | | | | |
| Phage- | 0.085 | 2.736 | 0.255 | 740 |
| 518 | | | | |
| Phage- | 0.063 | 2.763 | 0.237 | 741 |
| 519 | | | | |
| Phage- | 0.086 | 2.847 | 0.264 | 742 |
| 520 | | | | |
| Phage- | 0.062 | 2.784 | 0.238 | 743 |
| 521 | | | | |
| Phage- | 0.103 | 2.839 | 0.280 | 744 |
| 522 | | | | |
| Phage- | 0.155 | 2.824 | 0.327 | 745 |
| 523 | | | | |
| Phage- | 0.164 | 2.827 | 0.337 | 746 |
| 524 | | | | |
| Phage- | 0.132 | 2.741 | 0.301 | 747 |
| 525 | | | | |
| Phage- | 0.105 | 1.856 | 0.219 | 748 |
| 526 | | | | |
| Phage- | 0.091 | 2.718 | 0.263 | 749 |
| 527 | | | | |
| Phage- | 0.172 | 2.700 | 0.338 | 750 |
| 528 | | | | |
| Phage- | 0.111 | 2.687 | 0.280 | 751 |
| 529 | | | | |
| Phage- | 0.069 | 2.749 | 0.245 | 752 |
| 530 | | | | |
| Phage- | 0.084 | 2.755 | 0.261 | 753 |
| 531 | | | | |
| Phage- | 0.087 | 2.727 | 0.263 | 754 |
| 532 | | | | |
| Phage- | 0.141 | 2.694 | 0.312 | 755 |
| 533 | | | | |
| Phage- | 0.077 | 2.713 | 0.253 | 756 |
| 534 | | | | |
| Phage- | 0.153 | 2.730 | 0.325 | 757 |
| 535 | | | | |
| Phage- | 0.210 | 2.400 | 0.357 | 758 |
| 536 | | | | |
| Phage- | 0.217 | 2.731 | 0.386 | 759 |
| 537 | | | | |
| Phage- | 0.150 | 2.703 | 0.322 | 760 |
| 538 | | | | |
| Phage- | 0.384 | 2.714 | 0.542 | 761 |
| 539 | | | | |
| Phage- | 0.092 | 2.745 | 0.274 | 762 |
| 540 | | | | |
| Phage- | 0.186 | 2.749 | 0.362 | 763 |
| 541 | | | | |
| Phage- | 0.072 | 2.743 | 0.256 | 764 |
| 542 | | | | |
| Phage- | 0.154 | 2.718 | 0.330 | 765 |
| 543 | | | | |
| Phage- | 0.094 | 2.736 | 0.277 | 766 |
| 544 | | | | |
| Phage- | 0.102 | 2.755 | 0.285 | 767 |
| 545 | | | | |
| Phage- | 0.074 | 2.836 | 0.265 | 768 |
| 546 | | | | |
| Phage- | 0.096 | 2.750 | 0.282 | 769 |
| 547 | | | | |
| Phage- | 0.090 | 2.725 | 0.274 | 770 |
| 548 | | | | |
| Phage- | 0.103 | 2.838 | 0.296 | 771 |
| 549 | | | | |
| Phage- | 0.110 | 2.362 | 0.269 | 772 |
| 550 | | | | |
| Phage- | 0.124 | 2.723 | 0.308 | 773 |
| 551 | | | | |
| Phage- | 0.065 | 1.332 | 0.155 | 774 |
| 552 | | | | |
| Phage- | 0.073 | 2.702 | 0.259 | 775 |
| 553 | | | | |
| Phage- | 0.260 | 2.703 | 0.434 | 776 |
| 554 | | | | |
| Phage- | 0.151 | 2.837 | 0.344 | 777 |
| 555 | | | | |
| Phage- | 0.134 | 2.802 | 0.326 | 778 |
| 556 | | | | |
| Phage- | 0.198 | 2.676 | 0.377 | 779 |
| 557 | | | | |
| Phage- | 0.072 | 2.820 | 0.272 | 780 |
| 558 | | | | |
| Phage- | 0.142 | 2.696 | 0.329 | 781 |
| 559 | | | | |
| Phage- | 0.088 | 2.654 | 0.277 | 782 |
| 560 | | | | |
| Phage- | 0.092 | 2.746 | 0.287 | 783 |
| 561 | | | | |
| Phage- | 0.187 | 2.740 | 0.375 | 784 |
| 562 | | | | |
| Phage- | 0.069 | 2.321 | 0.236 | 785 |
| 563 | | | | |
| Phage- | 0.142 | 2.737 | 0.336 | 786 |
| 564 | | | | |
| Phage- | 0.079 | 2.679 | 0.273 | 787 |
| 565 | | | | |
| Phage- | 0.074 | 2.733 | 0.273 | 788 |
| 566 | | | | |
| Phage- | 0.163 | 2.695 | 0.353 | 789 |
| 567 | | | | |
| Phage- | 0.079 | 2.697 | 0.276 | 790 |
| 568 | | | | |
| Phage- | 0.090 | 2.738 | 0.288 | 791 |
| 569 | | | | |
| Phage- | 0.070 | 2.004 | 0.216 | 792 |
| 570 | | | | |
| Phage- | 0.431 | 2.750 | 0.606 | 793 |
| 571 | | | | |
| Phage- | 0.084 | 1.931 | 0.223 | 794 |
| 572 | | | | |
| Phage- | 0.176 | 2.753 | 0.370 | 795 |
| 573 | | | | |
| Phage- | 0.230 | 2.742 | 0.421 | 796 |
| 574 | | | | |
| Phage- | 0.139 | 2.755 | 0.340 | 797 |
| 575 | | | | |
| Phage- | 0.082 | 2.676 | 0.282 | 798 |
| 576 | | | | |
| Phage- | 0.105 | 2.697 | 0.305 | 799 |
| 577 | | | | |
| Phage- | 0.082 | 2.748 | 0.288 | 800 |
| 578 | | | | |
| Phage- | 0.124 | 2.833 | 0.335 | 801 |
| 579 | | | | |
| Phage- | 0.101 | 2.731 | 0.305 | 802 |
| 580 | | | | |
| Phage- | 0.082 | 2.732 | 0.288 | 803 |
| 581 | | | | |
| Phage- | 0.237 | 2.715 | 0.430 | 804 |
| 582 | | | | |
| Phage- | 0.327 | 2.697 | 0.512 | 805 |
| 583 | | | | |
| Phage- | 0.411 | 2.750 | 0.594 | 806 |
| 584 | | | | |
| Phage- | 0.132 | 2.588 | 0.326 | 807 |
| 585 | | | | |
| Phage- | 0.188 | 2.806 | 0.395 | 808 |
| 586 | | | | |
| Phage- | 0.102 | 2.192 | 0.272 | 809 |
| 587 | | | | |
| Phage- | 0.084 | 2.822 | 0.307 | 810 |
| 588 | | | | |
| Phage- | 0.072 | 2.743 | 0.289 | 811 |
| 589 | | | | |
| Phage- | 0.077 | 2.724 | 0.293 | 812 |
| 590 | | | | |
| Phage- | 0.076 | 2.132 | 0.244 | 813 |
| 591 | | | | |
| Phage- | 0.116 | 2.755 | 0.334 | 814 |
| 592 | | | | |
| Phage- | 0.182 | 2.829 | 0.401 | 815 |
| 593 | | | | |
| Phage- | 0.193 | 2.716 | 0.402 | 816 |
| 594 | | | | |
| Phage- | 0.064 | 1.493 | 0.182 | 817 |
| 595 | | | | |
| Phage- | 0.595 | 2.722 | 0.771 | 818 |
| 596 | | | | |
| Phage- | 0.151 | 2.827 | 0.375 | 819 |
| 597 | | | | |
| Phage- | 0.074 | 2.775 | 0.300 | 820 |
| 598 | | | | |
| Phage- | 0.108 | 2.656 | 0.323 | 821 |
| 599 | | | | |
| Phage- | 0.089 | 2.735 | 0.313 | 822 |
| 600 | | | | |
| Phage- | 0.173 | 2.760 | 0.392 | 823 |
| 601 | | | | |
| Phage- | 0.282 | 2.739 | 0.490 | 824 |
| 602 | | | | |
| Phage- | 0.250 | 2.708 | 0.459 | 825 |
| 603 | | | | |
| Phage- | 0.220 | 2.835 | 0.443 | 826 |
| 604 | | | | |
| Phage- | 0.117 | 2.698 | 0.337 | 827 |
| 605 | | | | |
| Phage- | 0.076 | 2.713 | 0.302 | 828 |
| 606 | | | | |
| Phage- | 0.070 | 2.740 | 0.299 | 829 |
| 607 | | | | |
| Phage- | 0.239 | 2.161 | 0.405 | 830 |
| 608 | | | | |
| Phage- | 0.220 | 2.763 | 0.444 | 831 |
| 609 | | | | |
| Phage- | 0.148 | 2.693 | 0.373 | 832 |
| 610 | | | | |
| Phage- | 0.156 | 2.742 | 0.386 | 833 |
| 611 | | | | |
| Phage- | 0.173 | 2.730 | 0.401 | 834 |
| 612 | | | | |
| Phage- | 0.074 | 2.752 | 0.313 | 835 |
| 613 | | | | |
| Phage- | 0.115 | 2.673 | 0.347 | 836 |
| 614 | | | | |
| Phage- | 0.135 | 2.832 | 0.381 | 837 |
| 615 | | | | |
| Phage- | 0.086 | 2.745 | 0.328 | 838 |
| 616 | | | | |
| Phage- | 0.121 | 2.747 | 0.361 | 839 |
| 617 | | | | |
| Phage- | 0.169 | 2.743 | 0.405 | 840 |
| 618 | | | | |
| Phage- | 0.103 | 2.042 | 0.281 | 841 |
| 619 | | | | |
| Phage- | 0.079 | 2.723 | 0.322 | 842 |
| 620 | | | | |
| Phage- | 0.094 | 2.843 | 0.346 | 843 |
| 621 | | | | |
| Phage- | 0.173 | 2.737 | 0.411 | 844 |
| 622 | | | | |
| Phage- | 0.130 | 2.734 | 0.372 | 845 |
| 623 | | | | |
| Phage- | 0.103 | 2.735 | 0.348 | 846 |
| 624 | | | | |
| Phage- | 0.113 | 2.740 | 0.360 | 847 |
| 625 | | | | |
| Phage- | 0.098 | 2.784 | 0.351 | 848 |
| 626 | | | | |
| Phage- | 0.076 | 2.844 | 0.337 | 849 |
| 627 | | | | |
| Phage- | 0.405 | 2.705 | 0.622 | 850 |
| 628 | | | | |
| Phage- | 0.204 | 2.764 | 0.445 | 851 |
| 629 | | | | |
| Phage- | 0.092 | 2.027 | 0.275 | 852 |
| 630 | | | | |
| Phage- | 0.132 | 2.714 | 0.376 | 853 |
| 631 | | | | |
| Phage- | 0.760 | 2.768 | 0.955 | 854 |
| 632 | | | | |
| Phage- | 0.258 | 2.738 | 0.499 | 855 |
| 633 | | | | |
| Phage- | 0.224 | 2.708 | 0.469 | 856 |
| 634 | | | | |
| Phage- | 0.213 | 2.697 | 0.461 | 857 |
| 635 | | | | |
| Phage- | 0.142 | 2.700 | 0.401 | 858 |
| 636 | | | | |
| Phage- | 0.146 | 2.718 | 0.409 | 859 |
| 637 | | | | |
| Phage- | 0.133 | 2.700 | 0.395 | 860 |
| 638 | | | | |
| Phage- | 0.225 | 2.846 | 0.494 | 861 |
| 639 | | | | |
| Phage- | 0.082 | 2.751 | 0.356 | 862 |
| 640 | | | | |
| Phage- | 0.068 | 2.180 | 0.287 | 863 |
| 641 | | | | |
| Phage- | 0.145 | 2.775 | 0.419 | 864 |
| 642 | | | | |
| Phage- | 0.134 | 2.536 | 0.386 | 865 |
| 643 | | | | |
| Phage- | 0.107 | 2.721 | 0.382 | 866 |
| 644 | | | | |
| Phage- | 0.069 | 1.074 | 0.176 | 867 |
| 645 | | | | |
| Phage- | 0.176 | 2.696 | 0.445 | 868 |
| 646 | | | | |
| Phage- | 0.074 | 2.821 | 0.368 | 869 |
| 647 | | | | |
| Phage- | 0.110 | 2.735 | 0.392 | 870 |
| 648 | | | | |
| Phage- | 0.203 | 2.737 | 0.475 | 871 |
| 649 | | | | |
| Phage- | 0.096 | 2.853 | 0.394 | 872 |
| 650 | | | | |
| Phage- | 0.193 | 2.683 | 0.463 | 873 |
| 651 | | | | |
| Phage- | 0.078 | 2.849 | 0.379 | 874 |
| 652 | | | | |
| Phage- | 0.103 | 2.743 | 0.392 | 875 |
| 653 | | | | |
| Phage- | 0.147 | 2.741 | 0.433 | 876 |
| 654 | | | | |
| Phage- | 0.121 | 2.750 | 0.411 | 877 |
| 655 | | | | |
| Phage- | 0.188 | 2.725 | 0.469 | 878 |
| 656 | | | | |
| Phage- | 0.120 | 2.647 | 0.400 | 879 |
| 657 | | | | |
| Phage- | 0.073 | 2.735 | 0.370 | 880 |
| 658 | | | | |
| Phage- | 0.170 | 2.712 | 0.455 | 881 |
| 659 | | | | |
| Phage- | 0.106 | 2.705 | 0.398 | 882 |
| 660 | | | | |
| Phage- | 0.166 | 2.713 | 0.453 | 883 |
| 661 | | | | |
| Phage- | 0.096 | 2.672 | 0.386 | 884 |
| 662 | | | | |
| Phage- | 0.182 | 2.674 | 0.463 | 885 |
| 663 | | | | |
| Phage- | 0.087 | 2.813 | 0.396 | 886 |
| 664 | | | | |
| Phage- | 0.092 | 2.730 | 0.391 | 887 |
| 665 | | | | |
| Phage- | 0.325 | 2.741 | 0.599 | 888 |
| 666 | | | | |
| Phage- | 0.110 | 2.721 | 0.407 | 889 |
| 667 | | | | |
| Phage- | 0.163 | 2.680 | 0.451 | 890 |
| 668 | | | | |
| Phage- | 0.282 | 2.609 | 0.550 | 891 |
| 669 | | | | |
| Phage- | 0.088 | 2.739 | 0.394 | 892 |
| 670 | | | | |
| Phage- | 0.147 | 2.853 | 0.461 | 893 |
| 671 | | | | |
| Phage- | 0.124 | 2.724 | 0.426 | 894 |
| 672 | | | | |
| Phage- | 0.198 | 2.824 | 0.503 | 895 |
| 673 | | | | |
| Phage- | 0.182 | 2.799 | 0.486 | 896 |
| 674 | | | | |
| Phage- | 0.097 | 0.831 | 0.183 | 897 |
| 675 | | | | |
| Phage- | 0.419 | 2.697 | 0.689 | 898 |
| 676 | | | | |
| Phage- | 0.130 | 2.764 | 0.443 | 899 |
| 677 | | | | |
| Phage- | 0.150 | 2.816 | 0.471 | 900 |
| 678 | | | | |
| Phage- | 0.115 | 2.720 | 0.430 | 901 |
| 679 | | | | |
| Phage- | 0.218 | 2.740 | 0.522 | 902 |
| 680 | | | | |
| Phage- | 0.145 | 2.714 | 0.456 | 903 |
| 681 | | | | |
| Phage- | 0.153 | 2.712 | 0.464 | 904 |
| 682 | | | | |
| Phage- | 0.083 | 2.696 | 0.403 | 905 |
| 683 | | | | |
| Phage- | 0.068 | 2.706 | 0.391 | 906 |
| 684 | | | | |
| Phage- | 0.087 | 2.709 | 0.409 | 907 |
| 685 | | | | |
| Phage- | 0.073 | 1.763 | 0.280 | 908 |
| 686 | | | | |
| Phage- | 0.113 | 2.824 | 0.446 | 909 |
| 687 | | | | |
| Phage- | 0.109 | 2.741 | 0.435 | 910 |
| 688 | | | | |
| Phage- | 0.088 | 2.754 | 0.419 | 911 |
| 689 | | | | |
| Phage- | 0.310 | 2.739 | 0.621 | 912 |
| 690 | | | | |
| Phage- | 0.150 | 2.735 | 0.482 | 913 |
| 691 | | | | |
| Phage- | 0.127 | 2.713 | 0.460 | 914 |
| 692 | | | | |
| Phage- | 0.141 | 2.732 | 0.475 | 915 |
| 693 | | | | |
| Phage- | 0.080 | 2.700 | 0.421 | 916 |
| 694 | | | | |
| Phage- | 0.316 | 2.691 | 0.625 | 917 |
| 695 | | | | |
| Phage- | 0.151 | 2.758 | 0.493 | 918 |
| 696 | | | | |
| Phage- | 0.082 | 2.625 | 0.419 | 919 |
| 697 | | | | |
| Phage- | 0.089 | 2.739 | 0.441 | 920 |
| 698 | | | | |
| Phage- | 0.342 | 2.683 | 0.654 | 921 |
| 699 | | | | |
| Phage- | 0.157 | 2.697 | 0.497 | 922 |
| 700 | | | | |
| Phage- | 0.136 | 2.723 | 0.487 | 923 |
| 701 | | | | |
| Phage- | 0.108 | 0.383 | 0.146 | 924 |
| 702 | | | | |
| Phage- | 0.105 | 2.755 | 0.470 | 925 |
| 703 | | | | |
| Phage- | 0.109 | 1.973 | 0.366 | 926 |
| 704 | | | | |
| Phage- | 0.147 | 2.683 | 0.497 | 927 |
| 705 | | | | |
| Phage- | 0.094 | 2.729 | 0.458 | 928 |
| 706 | | | | |
| Phage- | 0.156 | 2.704 | 0.508 | 929 |
| 707 | | | | |
| Phage- | 0.098 | 2.847 | 0.478 | 930 |
| 708 | | | | |
| Phage- | 0.094 | 2.711 | 0.456 | 931 |
| 709 | | | | |
| Phage- | 0.154 | 2.753 | 0.514 | 932 |
| 710 | | | | |
| Phage- | 0.075 | 2.731 | 0.444 | 933 |
| 711 | | | | |
| Phage- | 0.109 | 2.710 | 0.471 | 934 |
| 712 | | | | |
| Phage- | 0.153 | 2.845 | 0.529 | 935 |
| 713 | | | | |
| Phage- | 0.313 | 2.662 | 0.641 | 936 |
| 714 | | | | |
| Phage- | 0.352 | 2.710 | 0.684 | 937 |
| 715 | | | | |
| Phage- | 0.195 | 2.807 | 0.563 | 938 |
| 716 | | | | |
| Phage- | 0.180 | 2.727 | 0.541 | 939 |
| 717 | | | | |
| Phage- | 0.083 | 2.713 | 0.457 | 940 |
| 718 | | | | |
| Phage- | 0.125 | 2.746 | 0.498 | 941 |
| 719 | | | | |
| Phage- | 0.166 | 2.719 | 0.530 | 942 |
| 720 | | | | |
| Phage- | 0.274 | 2.707 | 0.626 | 943 |
| 721 | | | | |
| Phage- | 0.070 | 0.666 | 0.156 | 944 |
| 722 | | | | |
| Phage- | 0.131 | 2.696 | 0.505 | 945 |
| 723 | | | | |
| Phage- | 0.077 | 2.710 | 0.461 | 946 |
| 724 | | | | |
| Phage- | 0.300 | 2.749 | 0.658 | 947 |
| 725 | | | | |
| Phage- | 0.123 | 2.719 | 0.507 | 948 |
| 726 | | | | |
| Phage- | 0.073 | 2.656 | 0.455 | 949 |
| 727 | | | | |
| Phage- | 0.167 | 2.739 | 0.552 | 950 |
| 728 | | | | |
| Phage- | 0.126 | 2.709 | 0.512 | 951 |
| 729 | | | | |
| Phage- | 0.083 | 2.594 | 0.459 | 952 |
| 730 | | | | |
| Phage- | 0.104 | 2.728 | 0.498 | 953 |
| 731 | | | | |
| Phage- | 0.139 | 2.717 | 0.526 | 954 |
| 732 | | | | |
| Phage- | 0.086 | 2.745 | 0.486 | 955 |
| 733 | | | | |
| Phage- | 0.226 | 2.714 | 0.602 | 956 |
| 734 | | | | |
| Phage- | 0.131 | 2.723 | 0.524 | 957 |
| 735 | | | | |
| Phage- | 0.070 | 2.723 | 0.474 | 958 |
| 736 | | | | |
| Phage- | 0.159 | 2.753 | 0.555 | 959 |
| 737 | | | | |
| Phage- | 0.218 | 2.696 | 0.598 | 960 |
| 738 | | | | |
| Phage- | 0.078 | 2.741 | 0.488 | 961 |
| 739 | | | | |
| Phage- | 0.167 | 2.753 | 0.567 | 962 |
| 740 | | | | |
| Phage- | 0.349 | 2.716 | 0.717 | 963 |
| 741 | | | | |
| Phage- | 0.099 | 2.849 | 0.528 | 964 |
| 742 | | | | |
| Phage- | 0.202 | 2.721 | 0.598 | 965 |
| 743 | | | | |
| Phage- | 0.089 | 2.746 | 0.508 | 966 |
| 744 | | | | |
| Phage- | 0.086 | 0.545 | 0.159 | 967 |
| 745 | | | | |
| Phage- | 0.105 | 2.839 | 0.539 | 968 |
| 746 | | | | |
| Phage- | 0.106 | 2.851 | 0.543 | 969 |
| 747 | | | | |
| Phage- | 0.097 | 2.832 | 0.541 | 970 |
| 748 | | | | |
| Phage- | 0.194 | 2.721 | 0.605 | 971 |
| 749 | | | | |
| Phage- | 0.098 | 2.739 | 0.530 | 972 |
| 750 | | | | |
| Phage- | 0.077 | 2.547 | 0.483 | 973 |
| 751 | | | | |
| Phage- | 0.183 | 2.681 | 0.595 | 974 |
| 752 | | | | |
| Phage- | 0.310 | 2.838 | 0.734 | 975 |
| 753 | | | | |
| Phage- | 0.071 | 2.736 | 0.526 | 976 |
| 754 | | | | |
| Phage- | 0.063 | 0.767 | 0.183 | 977 |
| 755 | | | | |
| Phage- | 0.075 | 2.853 | 0.550 | 978 |
| 756 | | | | |
| Phage- | 0.150 | 2.711 | 0.589 | 979 |
| 757 | | | | |
| Phage- | 0.291 | 2.755 | 0.715 | 980 |
| 758 | | | | |
| Phage- | 0.441 | 2.714 | 0.839 | 981 |
| 759 | | | | |
| Phage- | 0.195 | 2.673 | 0.629 | 982 |
| 760 | | | | |
| Phage- | 0.123 | 2.737 | 0.583 | 983 |
| 761 | | | | |
| Phage- | 0.311 | 2.725 | 0.738 | 984 |
| 762 | | | | |
| Phage- | 0.095 | 2.737 | 0.563 | 985 |
| 763 | | | | |
| Phage- | 0.090 | 2.706 | 0.554 | 986 |
| 764 | | | | |
| Phage- | 0.102 | 2.753 | 0.573 | 987 |
| 765 | | | | |
| Phage- | 0.228 | 2.753 | 0.678 | 988 |
| 766 | | | | |
| Phage- | 0.171 | 2.719 | 0.627 | 989 |
| 767 | | | | |
| Phage- | 0.182 | 2.700 | 0.637 | 990 |
| 768 | | | | |
| Phage- | 0.112 | 2.753 | 0.590 | 991 |
| 769 | | | | |
| Phage- | 0.085 | 2.737 | 0.566 | 992 |
| 770 | | | | |
| Phage- | 0.085 | 2.744 | 0.568 | 993 |
| 771 | | | | |
| Phage- | 0.239 | 2.826 | 0.710 | 994 |
| 772 | | | | |
| Phage- | 0.067 | 2.724 | 0.557 | 995 |
| 773 | | | | |
| Phage- | 0.142 | 2.833 | 0.640 | 996 |
| 774 | | | | |
| Phage- | 0.192 | 2.780 | 0.671 | 997 |
| 775 | | | | |
| Phage- | 0.462 | 2.755 | 0.887 | 998 |
| 776 | | | | |
| Phage- | 0.095 | 2.724 | 0.583 | 999 |
| 777 | | | | |
| Phage- | 0.187 | 2.858 | 0.687 | 1000 |
| 778 | | | | |
| Phage- | 0.176 | 2.759 | 0.659 | 1001 |
| 779 | | | | |
| Phage- | 0.250 | 2.831 | 0.735 | 1002 |
| 780 | | | | |
| Phage- | 0.109 | 0.631 | 0.207 | 1003 |
| 781 | | | | |
| Phage- | 0.093 | 2.780 | 0.599 | 1004 |
| 782 | | | | |
| Phage- | 0.433 | 2.737 | 0.869 | 1005 |
| 783 | | | | |
| Phage- | 0.406 | 2.818 | 0.862 | 1006 |
| 784 | | | | |
| Phage- | 0.121 | 2.837 | 0.639 | 1007 |
| 785 | | | | |
| Phage- | 0.111 | 2.754 | 0.619 | 1008 |
| 786 | | | | |
| Phage- | 0.092 | 2.743 | 0.603 | 1009 |
| 787 | | | | |
| Phage- | 0.097 | 2.849 | 0.630 | 1010 |
| 788 | | | | |
| Phage- | 0.139 | 2.839 | 0.663 | 1011 |
| 789 | | | | |
| Phage- | 0.080 | 2.788 | 0.612 | 1012 |
| 790 | | | | |
| Phage- | 0.171 | 2.724 | 0.672 | 1013 |
| 791 | | | | |
| Phage- | 0.094 | 2.708 | 0.612 | 1014 |
| 792 | | | | |
| Phage- | 0.386 | 2.858 | 0.877 | 1015 |
| 793 | | | | |
| Phage- | 0.343 | 2.832 | 0.846 | 1016 |
| 794 | | | | |
| Phage- | 0.103 | 2.869 | 0.663 | 1017 |
| 795 | | | | |
| Phage- | 0.105 | 2.832 | 0.660 | 1018 |
| 796 | | | | |
| Phage- | 0.099 | 2.729 | 0.635 | 1019 |
| 797 | | | | |
| Phage- | 0.291 | 2.732 | 0.791 | 1020 |
| 798 | | | | |
| Phage- | 0.098 | 2.823 | 0.658 | 1021 |
| 799 | | | | |
| Phage- | 0.145 | 2.691 | 0.682 | 1022 |
| 800 | | | | |
| Phage- | 0.073 | 2.735 | 0.639 | 1023 |
| 801 | | | | |
| Phage- | 0.136 | 2.735 | 0.697 | 1024 |
| 802 | | | | |
| Phage- | 0.074 | 2.721 | 0.646 | 1025 |
| 803 | | | | |
| Phage- | 0.709 | 2.687 | 1.137 | 1026 |
| 804 | | | | |
| Phage- | 0.115 | 0.066 | 0.105 | 1027 |
| 805 | | | | |
| Phage- | 0.824 | 2.717 | 1.236 | 1028 |
| 806 | | | | |
| Phage- | 0.330 | 2.727 | 0.851 | 1029 |
| 807 | | | | |
| Phage- | 0.093 | 2.824 | 0.689 | 1030 |
| 808 | | | | |
| Phage- | 0.506 | 2.727 | 0.995 | 1031 |
| 809 | | | | |
| Phage- | 0.142 | 2.721 | 0.719 | 1032 |
| 810 | | | | |
| Phage- | 0.084 | 2.740 | 0.678 | 1033 |
| 811 | | | | |
| Phage- | 0.079 | 2.760 | 0.681 | 1034 |
| 812 | | | | |
| Phage- | 1.432 | 2.725 | 1.725 | 1035 |
| 813 | | | | |
| Phage- | 0.090 | 2.413 | 0.617 | 1036 |
| 814 | | | | |
| Phage- | 0.075 | 2.843 | 0.711 | 1037 |
| 815 | | | | |
| Phage- | 0.070 | 2.719 | 0.680 | 1038 |
| 816 | | | | |
| Phage- | 0.090 | 2.718 | 0.698 | 1039 |
| 817 | | | | |
| Phage- | 0.761 | 2.730 | 1.218 | 1040 |
| 818 | | | | |
| Phage- | 0.079 | 0.186 | 0.103 | 1041 |
| 819 | | | | |
| Phage- | 0.621 | 2.743 | 1.115 | 1042 |
| 820 | | | | |
| Phage- | 0.131 | 2.708 | 0.732 | 1043 |
| 821 | | | | |
| Phage- | 0.121 | 2.687 | 0.729 | 1044 |
| 822 | | | | |
| Phage- | 0.099 | 0.088 | 0.096 | 1045 |
| 823 | | | | |
| Phage- | 0.181 | 2.751 | 0.799 | 1046 |
| 824 | | | | |
| Phage- | 0.288 | 2.742 | 0.881 | 1047 |
| 825 | | | | |
| Phage- | 0.106 | 2.737 | 0.742 | 1048 |
| 826 | | | | |
| Phage- | 0.154 | 2.760 | 0.790 | 1049 |
| 827 | | | | |
| Phage- | 0.078 | 2.641 | 0.704 | 1050 |
| 828 | | | | |
| Phage- | 0.870 | 2.714 | 1.321 | 1051 |
| 829 | | | | |
| Phage- | 0.578 | 2.745 | 1.115 | 1052 |
| 830 | | | | |
| Phage- | 0.369 | 2.819 | 0.978 | 1053 |
| 831 | | | | |
| Phage- | 0.116 | 2.845 | 0.796 | 1054 |
| 832 | | | | |
| Phage- | 0.108 | 2.748 | 0.766 | 1055 |
| 833 | | | | |
| Phage- | 0.104 | 2.741 | 0.765 | 1056 |
| 834 | | | | |
| Phage- | 0.083 | 0.755 | 0.255 | 1057 |
| 835 | | | | |
| Phage- | 0.077 | 2.849 | 0.795 | 1058 |
| 836 | | | | |
| Phage- | 0.084 | 2.733 | 0.772 | 1059 |
| 837 | | | | |
| Phage- | 0.259 | 2.752 | 0.908 | 1060 |
| 838 | | | | |
| Phage- | 0.154 | 2.746 | 0.830 | 1061 |
| 839 | | | | |
| Phage- | 0.085 | 2.745 | 0.781 | 1062 |
| 840 | | | | |
| Phage- | 0.294 | 2.098 | 0.768 | 1063 |
| 841 | | | | |
| Phage- | 2.648 | 2.751 | 2.675 | 1064 |
| 842 | | | | |
| Phage- | 0.183 | 2.746 | 0.860 | 1065 |
| 843 | | | | |
| Phage- | 0.281 | 2.762 | 0.947 | 1066 |
| 844 | | | | |
| Phage- | 0.207 | 2.717 | 0.881 | 1067 |
| 845 | | | | |
| Phage- | 0.625 | 2.711 | 1.186 | 1068 |
| 846 | | | | |
| Phage- | 0.727 | 0.580 | 0.686 | 1069 |
| 847 | | | | |
| Phage- | 0.141 | 2.764 | 0.863 | 1070 |
| 848 | | | | |
| Phage- | 0.193 | 2.736 | 0.901 | 1071 |
| 849 | | | | |
| Phage- | 0.409 | 2.621 | 1.042 | 1072 |
| 850 | | | | |
| Phage- | 0.234 | 2.827 | 0.979 | 1073 |
| 851 | | | | |
| Phage- | 0.086 | 0.066 | 0.080 | 1074 |
| 852 | | | | |
| Phage- | 0.116 | 2.838 | 0.927 | 1075 |
| 853 | | | | |
| Phage- | 0.147 | 2.815 | 0.951 | 1076 |
| 854 | | | | |
| Phage- | 0.078 | 2.754 | 0.888 | 1077 |
| 855 | | | | |
| Phage- | 0.086 | 2.717 | 0.887 | 1078 |
| 856 | | | | |
| Phage- | 0.099 | 2.729 | 0.902 | 1079 |
| 857 | | | | |
| Phage- | 0.103 | 2.870 | 0.948 | 1080 |
| 858 | | | | |
| Phage- | 0.082 | 2.774 | 0.923 | 1081 |
| 859 | | | | |
| Phage- | 0.117 | 2.736 | 0.940 | 1082 |
| 860 | | | | |
| Phage- | 0.184 | 0.066 | 0.147 | 1083 |
| 861 | | | | |
| Phage- | 0.116 | 2.729 | 0.938 | 1084 |
| 862 | | | | |
| Phage- | 0.506 | 2.748 | 1.220 | 1085 |
| 863 | | | | |
| Phage- | 0.066 | 2.329 | 0.787 | 1086 |
| 864 | | | | |
| Phage- | 0.162 | 2.753 | 0.999 | 1087 |
| 865 | | | | |
| Phage- | 0.098 | 2.750 | 0.957 | 1088 |
| 866 | | | | |
| Phage- | 0.488 | 2.730 | 1.221 | 1089 |
| 867 | | | | |
| Phage- | 0.089 | 0.076 | 0.084 | 1090 |
| 868 | | | | |
| Phage- | 0.187 | 2.811 | 1.059 | 1091 |
| 869 | | | | |
| Phage- | 0.089 | 2.731 | 0.974 | 1092 |
| 870 | | | | |
| Phage- | 0.065 | 2.730 | 0.960 | 1093 |
| 871 | | | | |
| Phage- | 0.713 | 2.748 | 1.397 | 1094 |
| 872 | | | | |
| Phage- | 0.068 | 2.722 | 0.962 | 1095 |
| 873 | | | | |
| Phage- | 0.103 | 2.723 | 0.992 | 1096 |
| 874 | | | | |
| Phage- | 0.365 | 2.707 | 1.161 | 1097 |
| 875 | | | | |
| Phage- | 0.186 | 0.093 | 0.154 | 1098 |
| 876 | | | | |
| Phage- | 0.116 | 2.725 | 1.020 | 1099 |
| 877 | | | | |
| Phage- | 0.320 | 2.752 | 1.168 | 1100 |
| 878 | | | | |
| Phage- | 0.100 | 2.727 | 1.017 | 1101 |
| 879 | | | | |
| Phage- | 0.092 | 0.078 | 0.087 | 1102 |
| 880 | | | | |
| Phage- | 0.288 | 2.839 | 1.183 | 1103 |
| 881 | | | | |
| Phage- | 0.081 | 2.837 | 1.056 | 1104 |
| 882 | | | | |
| Phage- | 0.076 | 2.709 | 1.015 | 1105 |
| 883 | | | | |
| Phage- | 0.145 | 2.727 | 1.069 | 1106 |
| 884 | | | | |
| Phage- | 0.133 | 2.721 | 1.063 | 1107 |
| 885 | | | | |
| Phage- | 0.093 | 2.754 | 1.055 | 1108 |
| 886 | | | | |
| Phage- | 0.272 | 2.681 | 1.151 | 1109 |
| 887 | | | | |
| Phage- | 2.625 | 2.705 | 2.654 | 1110 |
| 888 | | | | |
| Phage- | 0.160 | 2.709 | 1.097 | 1111 |
| 889 | | | | |
| Phage- | 0.362 | 2.716 | 1.249 | 1112 |
| 890 | | | | |
| Phage- | 0.107 | 0.079 | 0.096 | 1113 |
| 891 | | | | |
| Phage- | 0.098 | 2.719 | 1.136 | 1114 |
| 892 | | | | |
| Phage- | 0.103 | 2.706 | 1.139 | 1115 |
| 893 | | | | |
| Phage- | 0.133 | 0.088 | 0.114 | 1116 |
| 894 | | | | |
| Phage- | 0.414 | 2.732 | 1.375 | 1117 |
| 895 | | | | |
| Phage- | 0.138 | 2.753 | 1.227 | 1118 |
| 896 | | | | |
| Phage- | 0.444 | 2.746 | 1.404 | 1119 |
| 897 | | | | |
| Phage- | 2.674 | 2.737 | 2.701 | 1120 |
| 898 | | | | |
| Phage- | 0.233 | 2.708 | 1.286 | 1121 |
| 899 | | | | |
| Phage- | 0.098 | 2.807 | 1.254 | 1122 |
| 900 | | | | |
| Phage- | 0.141 | 2.834 | 1.298 | 1123 |
| 901 | | | | |
| Phage- | 0.291 | 0.077 | 0.198 | 1124 |
| 902 | | | | |
| Phage- | 0.567 | 2.835 | 1.582 | 1125 |
| 903 | | | | |
| Phage- | 0.448 | 2.791 | 1.507 | 1126 |
| 904 | | | | |
| Phage- | 0.202 | 2.747 | 1.388 | 1127 |
| 905 | | | | |
| Phage- | 0.593 | 2.743 | 1.595 | 1128 |
| 906 | | | | |
| Phage- | 0.184 | 2.716 | 1.369 | 1129 |
| 907 | | | | |
| Phage- | 0.091 | 0.078 | 0.085 | 1130 |
| 908 | | | | |
| Phage- | 0.079 | 2.712 | 1.373 | 1131 |
| 909 | | | | |
| Phage- | 0.107 | 0.078 | 0.092 | 1132 |
| 910 | | | | |
| Phage- | 0.205 | 2.852 | 1.509 | 1133 |
| 911 | | | | |
| Phage- | 2.238 | 2.758 | 2.500 | 1134 |
| 912 | | | | |
| Phage- | 0.064 | 0.106 | 0.085 | 1135 |
| 913 | | | | |
| Phage- | 0.363 | 0.074 | 0.214 | 1136 |
| 914 | | | | |
| Phage- | 0.131 | 2.847 | 1.543 | 1137 |
| 915 | | | | |
| Phage- | 0.096 | 0.079 | 0.087 | 1138 |
| 916 | | | | |
| Phage- | 0.792 | 2.762 | 1.822 | 1139 |
| 917 | | | | |
| Phage- | 0.356 | 2.688 | 1.580 | 1140 |
| 918 | | | | |
| Phage- | 0.193 | 0.076 | 0.131 | 1141 |
| 919 | | | | |
| Phage- | 2.542 | 2.739 | 2.649 | 1142 |
| 920 | | | | |
| Phage- | 2.282 | 2.794 | 2.572 | 1143 |
| 921 | | | | |
| Phage- | 0.636 | 2.717 | 1.844 | 1144 |
| 922 | | | | |
| Phage- | 2.598 | 2.730 | 2.679 | 1145 |
| 923 | | | | |
| Phage- | 0.184 | 2.742 | 1.803 | 1146 |
| 924 | | | | |
| Phage- | 0.816 | 2.745 | 2.039 | 1147 |
| 925 | | | | |
| Phage- | 0.735 | 2.756 | 2.019 | 1148 |
| 926 | | | | |
| Phage- | 0.479 | 2.699 | 1.911 | 1149 |
| 927 | | | | |
| Phage- | 0.375 | 0.074 | 0.172 | 1150 |
| 928 | | | | |
| Phage- | 0.169 | 0.067 | 0.100 | 1151 |
| 929 | | | | |
| Phage- | 2.316 | 2.823 | 2.660 | 1152 |
| 930 | | | | |
| Phage- | 0.765 | 2.845 | 2.181 | 1153 |
| 931 | | | | |
| Phage- | 0.163 | 0.080 | 0.106 | 1154 |
| 932 | | | | |
| Phage- | 0.295 | 2.745 | 1.989 | 1155 |
| 933 | | | | |
| Phage- | 0.192 | 0.082 | 0.114 | 1156 |
| 934 | | | | |
| Phage- | 0.075 | 0.066 | 0.069 | 1157 |
| 935 | | | | |
| Phage- | 1.474 | 2.816 | 2.447 | 1158 |
| 936 | | | | |
| Phage- | 0.769 | 2.717 | 2.218 | 1159 |
| 937 | | | | |
| Phage- | 0.609 | 2.745 | 2.200 | 1160 |
| 938 | | | | |
| Phage- | 1.479 | 2.733 | 2.413 | 1161 |
| 939 | | | | |
| Phage- | 0.220 | 0.079 | 0.113 | 1162 |
| 940 | | | | |
| Phage- | 0.106 | 2.863 | 2.262 | 1163 |
| 941 | | | | |
| Phage- | 0.161 | 0.064 | 0.085 | 1164 |
| 942 | | | | |
| Phage- | 1.234 | 2.743 | 2.430 | 1165 |
| 943 | | | | |
| Phage- | 0.073 | 0.115 | 0.106 | 1166 |
| 944 | | | | |
| Phage- | 0.117 | 0.064 | 0.075 | 1167 |
| 945 | | | | |
| Phage- | 0.967 | 2.861 | 2.503 | 1168 |
| 946 | | | | |
| Phage- | 0.731 | 2.735 | 2.357 | 1169 |
| 947 | | | | |
| Phage- | 0.246 | 0.082 | 0.113 | 1170 |
| 948 | | | | |
| Phage- | 0.479 | 2.690 | 2.290 | 1171 |
| 949 | | | | |
| Phage- | 0.274 | 2.747 | 2.304 | 1172 |
| 950 | | | | |
| Phage- | 0.117 | 0.065 | 0.074 | 1173 |
| 951 | | | | |
| Phage- | 0.200 | 0.066 | 0.085 | 1174 |
| 952 | | | | |
| Phage- | 0.064 | 0.378 | 0.335 | 1175 |
| 953 | | | | |
| Phage- | 2.463 | 2.777 | 2.734 | 1176 |
| 954 | | | | |
| Phage- | 1.900 | 2.747 | 2.634 | 1177 |
| 955 | | | | |
| Phage- | 0.070 | 0.065 | 0.065 | 1178 |
| 956 | | | | |
| Phage- | 0.449 | 0.072 | 0.120 | 1179 |
| 957 | | | | |
| Phage- | 1.318 | 2.708 | 2.533 | 1180 |
| 958 | | | | |
| Phage- | 2.109 | 2.706 | 2.632 | 1181 |
| 959 | | | | |
| Phage- | 1.469 | 2.850 | 2.688 | 1182 |
| 960 | | | | |
| Phage- | 2.104 | 2.733 | 2.665 | 1183 |
| 961 | | | | |
| Phage- | 0.823 | 2.672 | 2.471 | 1184 |
| 962 | | | | |
| Phage- | 0.765 | 2.831 | 2.645 | 1185 |
| 963 | | | | |
| Phage- | 0.202 | 0.067 | 0.079 | 1186 |
| 964 | | | | |
| Phage- | 1.392 | 2.711 | 2.605 | 1187 |
| 965 | | | | |
| Phage- | 0.467 | 0.081 | 0.098 | 1188 |
| 966 | | | | |
| Phage- | 0.215 | 2.726 | 2.636 | 1189 |
| 967 | | | | |
| Phage- | 0.748 | 2.719 | 2.701 | 1190 |
| 968 | | | | |
| Phage- | 0.092 | 0.692 | 0.781 | 1191 |
| 969 | | | | |
| Phage- | 0.074 | 0.381 | 1.203 | 1192 |
| 970 | | | | |
| Phage- | 0.317 | 0.294 | 0.100 | 1193 |
| 971 | | | | |
| Phage- | 0.105 | 0.112 | 0.133 | 1194 |
| 972 | | | | |
| Phage- | 0.138 | 0.107 | 0.099 | 1195 |
| 973 | | | | |
| Phage- | 0.063 | 0.086 | 0.096 | 1196 |
| 974 | | | | |
| Phage- | 0.079 | 0.081 | 0.113 | 1197 |
| 975 | | | | |
| Phage- | 0.071 | 0.074 | 0.081 | 1198 |
| 976 | | | | |
| Phage- | 0.068 | 0.072 | 0.109 | 1199 |
| 977 | | | | |
| Phage- | 0.234 | 0.068 | 0.066 | 1200 |
| 978 | | | | |
| Phage- | 0.064 | 0.065 | 0.076 | 1201 |
| 979 | | | | |
| Phage- | 0.104 | 0.065 | 0.065 | 1202 |
| 980 | | | | |

TABLE 28
Sequences of those peptides selected for
synthesis (TROP2 Fab Peptide-1 Optimization)

| Construct | Amino | |
|---|---|---|
| Description | Acid Sequence | SEQ ID NO: |
| Peptide-30 | DALICVKNLFCWTS | 159 |
| Peptide-31 | DNLICVKNLWCWIA | 160 |
| Peptide-32 | DTLFCVKNLYCWIV | 161 |
| Peptide-33 | DTLSCFRNLYCWIT | 162 |
| Peptide-34 | DTLWCVKNLYCWVA | 163 |
| Peptide-35 | DVLFCVRNLYCWTS | 164 |
| Peptide-36 | FDLLCVRNLYCWNV | 165 |
| Peptide-37 | FSLVCVRNLYCWNV | 166 |
| Peptide-38 | FTLFCVQNLYCWNV | 167 |
| Peptide-39 | HDLQCFQNLFCWIV | 168 |
| Peptide-40 | LSLFCVKNLYCWNV | 169 |
| Peptide-41 | NYLLCVKNLYCWIV | 170 |
| Peptide-42 | STLECVRNLYCWIS | 171 |
| Peptide-43 | STLFCVKNLYCWVA | 172 |
| Peptide-44 | STLFCVRNLYCWTV | 173 |
| Peptide-45 | SYLVCVKNVYCWTA | 174 |
| Peptide-46 | TALFCFKNVYCWNV | 175 |
| Peptide-47 | TSLICFRNVYCWNV | 176 |
| Peptide-48 | YDLVCLRNLFCWTA | 177 |
| Peptide-49 | YSLVCVKNLYCWNL | 178 |

TABLE 29
Phage panning results of TROP2 Fab Peptide-2 library sequences.

| | | | | | | | | | | | | | | | Phage binding ELISA | | | |
|---|---|---|---|---|---|---|---|---|---|---|---|---|---|---|---|---|---|---|
| | | | | | | | | | | | | | | | | | TROP2 | |
| | | | | | | | | | | | | | | | | | Fab | |
| | | | | | | | | | | | | | | | | | signal in | |
| | | | | | | | | | | | | | | | | TROP2 | presence | SEQ |
| | Amino acid position sequence | | | | | | | | | | | | | | Backgroud | Fab | of | ID |
| Phage ID | 1 | 2 | 3 | 4 | 5 | 6 | 7 | 8 | 9 | 10 | 11 | 12 | 13 | 14 | signal | signal | TROP2 | NO: |
| Phage-981 | V | D | F | C | K | I | Y | S | W | P | V | C | H | Q | 0.055 | 2.832 | 0.067 | 24 |
| Phage-982 | I | — | — | — | A | M | — | Q | — | — | I | — | D | T | 0.058 | 2.945 | 0.064 | 179 |
| Phage-983 | I | — | — | — | A | V | — | K | — | — | — | — | Q | V | 0.059 | 2.885 | 0.064 | 180 |
| Phage-984 | I | — | — | — | M | L | — | N | — | — | I | — | A | G | 0.061 | 2.933 | 0.064 | 25 |
| Phage-985 | — | — | — | — | — | — | — | A | — | — | I | — | G | S | 0.066 | 2.868 | 0.068 | 182 |
| Phage-986 | — | — | — | — | — | L | — | N | — | — | — | — | Q | T | 0.061 | 2.874 | 0.067 | 183 |
| Phage-987 | I | — | — | — | L | — | — | N | — | — | — | — | D | T | 0.060 | 2.849 | 0.068 | 184 |
| Phage-988 | E | — | — | — | — | L | — | N | — | — | I | — | Y | — | 0.054 | 2.864 | 0.099 | 185 |
| Phage-989 | — | — | — | — | G | L | — | H | — | — | I | — | Y | — | 0.079 | 2.878 | 0.097 | 186 |
| Phage-990 | — | — | — | — | Y | L | — | N | — | — | — | — | S | K | 0.057 | 2.892 | 0.063 | 187 |
| Phage-991 | I | — | — | — | A | — | — | Q | — | — | — | — | R | S | 0.066 | 2.799 | 0.067 | 188 |
| Phage-992 | — | — | — | — | A | L | — | N | — | — | — | — | E | T | 0.056 | 2.752 | 0.058 | 189 |
| Phage-993 | P | — | — | — | A | V | — | R | — | — | I | — | Y | — | 0.062 | 2.746 | 0.073 | 190 |
| Phage-994 | — | — | — | — | E | L | — | R | — | — | I | — | N | S | 0.066 | 2.756 | 0.057 | 191 |
| Phage-995 | L | — | — | — | — | — | — | D | — | — | I | — | — | L | 0.082 | 2.760 | 0.083 | 192 |
| Phage-996 | L | — | — | — | — | L | — | Q | — | — | — | — | F | T | 0.080 | 2.763 | 0.079 | 193 |
| Phage-997 | I | — | — | — | L | L | — | D | — | — | — | — | A | S | 0.058 | 2.873 | 0.065 | 194 |
| Phage-998 | I | — | — | — | L | L | — | D | — | — | I | — | G | R | 0.170 | 2.774 | 0.205 | 195 |
| Phage-999 | M | — | — | — | Q | — | — | D | — | — | I | — | R | L | 0.057 | 2.589 | 0.062 | 196 |
| Phage-1000 | P | — | — | — | Q | L | — | N | — | — | — | — | A | G | 0.067 | 2.523 | 0.062 | 197 |
| Phage-1001 | — | — | — | — | S | F | — | R | — | — | I | — | E | T | 0.063 | 2.697 | 0.070 | 198 |
| Phage-1002 | I | — | — | — | S | L | — | Q | — | — | — | — | G | T | 0.063 | 2.828 | 0.091 | 199 |
| Phage-1003 | — | — | — | — | T | — | — | K | — | — | — | — | E | G | 0.055 | 2.760 | 0.059 | 200 |
| Phage-1004 | — | — | — | — | Y | Q | — | G | — | — | I | — | S | R | 0.060 | 2.696 | 0.060 | 201 |
| Phage-1005 | — | — | — | — | A | — | F | N | — | — | — | — | S | S | 0.063 | 2.742 | 0.068 | 1203 |
| Phage-1006 | L | — | — | — | A | — | — | K | — | — | — | — | F | E | 0.057 | 2.901 | 0.063 | 1204 |
| Phage-1007 | — | — | — | — | A | — | — | — | — | — | I | — | K | I | 0.066 | 2.834 | 0.064 | 1205 |
| Phage-1008 | I | — | — | — | A | — | — | P | — | — | I | — | D | T | 0.059 | 2.852 | 0.058 | 1206 |
| Phage-1009 | I | — | — | — | A | — | — | H | — | — | — | — | T | S | 0.057 | 2.707 | 0.063 | 1207 |
| Phage-1010 | I | — | — | — | A | — | — | Q | — | — | — | — | K | S | 0.108 | 2.810 | 0.096 | 1208 |
| Phage-1011 | M | — | — | — | A | — | — | H | — | — | — | — | N | Y | 0.061 | 2.760 | 0.062 | 1209 |
| Phage-1012 | E | — | — | — | A | — | — | R | — | — | — | — | L | T | 0.055 | 2.742 | 0.056 | 1210 |
| Phage-1013 | — | — | — | — | A | — | — | G | — | — | — | — | D | I | 0.068 | 1.940 | 0.067 | 1211 |
| Phage-1014 | F | — | — | — | A | — | — | R | — | — | — | — | Y | D | 0.072 | 1.407 | 0.080 | 1212 |
| Phage-1015 | — | — | — | — | A | K | — | E | — | — | I | — | S | T | 0.054 | 2.391 | 0.056 | 1213 |
| Phage-1016 | — | — | — | — | A | L | — | A | — | — | — | — | N | V | 0.060 | 2.810 | 0.057 | 1214 |
| Phage-1017 | I | — | — | — | A | L | — | — | — | — | — | — | N | S | 0.057 | 2.855 | 0.071 | 1215 |
| Phage-1018 | L | — | — | — | A | L | — | D | — | — | — | — | R | N | 0.062 | 2.717 | 0.069 | 1216 |
| Phage-1019 | — | — | — | — | A | L | — | H | — | — | — | — | G | L | 0.065 | 2.770 | 0.065 | 1217 |
| Phage-1020 | L | — | — | — | A | L | — | A | — | — | — | — | N | M | 0.059 | 2.649 | 0.071 | 1218 |
| Phage-1021 | L | — | — | — | A | L | N | A | — | — | — | — | N | M | 0.062 | 2.241 | 0.064 | 1219 |
| Phage-1022 | — | — | — | — | A | L | — | G | — | — | — | — | Y | L | 0.065 | 2.710 | 0.062 | 1220 |
| Phage-1023 | L | — | — | — | A | L | — | G | — | — | I | — | S | A | 0.055 | 2.208 | 0.059 | 1221 |
| Phage-1024 | — | — | — | — | A | L | — | N | — | — | — | — | Q | T | 0.061 | 2.848 | 0.059 | 1222 |
| Phage-1025 | I | — | — | — | A | L | — | R | — | — | — | — | Y | M | 0.136 | 2.760 | 0.148 | 1223 |
| Phage-1026 | — | — | — | — | A | L | — | E | — | — | I | — | M | M | 0.068 | 2.709 | 0.078 | 1224 |
| Phage-1027 | L | — | — | — | A | L | — | N | — | — | — | — | N | I | 0.063 | 2.625 | 0.073 | 1225 |
| Phage-1028 | — | — | — | — | A | L | — | N | — | — | — | — | D | T | 0.056 | 2.625 | 0.058 | 1226 |
| Phage-1029 | — | — | — | — | A | L | — | A | — | — | — | — | M | Y | 0.066 | 2.553 | 0.066 | 1227 |
| Phage-1030 | L | — | — | — | A | L | — | A | — | — | — | — | L | G | 0.059 | 2.401 | 0.064 | 1228 |
| Phage-1031 | — | — | — | — | A | L | F | E | — | — | I | — | G | A | 0.057 | 2.354 | 0.065 | 1229 |
| Phage-1032 | — | — | — | — | A | L | — | M | — | — | — | — | R | D | 0.066 | 2.270 | 0.073 | 1230 |
| Phage-1033 | — | — | W | — | A | L | — | K | — | — | — | — | S | T | 0.079 | 2.239 | 0.097 | 1231 |
| Phage-1034 | — | — | W | — | A | L | — | — | — | — | — | — | S | H | 0.069 | 2.091 | 0.070 | 1232 |
| Phage-1035 | L | G | — | — | A | L | N | G | — | — | I | — | S | A | 0.082 | 1.816 | 0.077 | 1233 |
| Phage-1036 | L | — | — | — | A | L | N | D | — | — | — | — | K | E | 0.055 | 1.662 | 0.062 | 1234 |
| Phage-1037 | I | — | — | — | A | L | — | G | — | — | — | — | D | T | 0.073 | 1.576 | 0.068 | 1235 |
| Phage-1038 | — | — | — | — | A | L | — | L | — | — | I | — | N | — | 0.054 | 1.567 | 0.062 | 1236 |
| Phage-1039 | — | — | — | — | A | L | — | M | — | — | — | — | Q | R | 0.055 | 1.464 | 0.059 | 1237 |
| Phage-1040 | L | — | — | — | A | L | N | G | — | — | I | — | S | A | 0.058 | 1.072 | 0.056 | 1238 |
| Phage-1041 | E | — | — | — | A | L | — | K | — | — | — | — | R | T | 0.059 | 0.658 | 0.059 | 1239 |
| Phage-1042 | I | — | — | — | A | M | — | H | — | — | — | — | D | T | 0.067 | 2.733 | 0.062 | 1240 |
| Phage-1043 | I | — | — | — | A | M | F | — | — | — | — | — | F | T | 0.085 | 2.836 | 0.082 | 1241 |
| Phage-1044 | — | — | — | — | A | M | — | H | — | — | I | — | Y | — | 0.062 | 2.771 | 0.067 | 1242 |
| Phage-1045 | 1 | — | — | — | A | M | — | Q | — | — | I | — | Y | T | 0.056 | 2.674 | 0.062 | 1243 |
| Phage-1046 | I | — | — | — | A | M | — | Q | — | — | I | — | V | A | 0.072 | 2.525 | 0.070 | 1244 |
| Phage-1047 | E | — | — | — | A | M | — | Y | — | — | I | — | G | L | 0.054 | 2.112 | 0.061 | 1245 |
| Phage-1048 | — | — | — | — | A | M | — | K | — | — | — | — | I | G | 0.081 | 2.053 | 0.066 | 1246 |
| Phage-1049 | I | — | — | — | A | M | — | Q | — | — | I | — | Q | T | 0.054 | 1.944 | 0.056 | 1247 |
| Phage-1050 | E | — | — | — | A | M | — | Q | — | — | — | — | I | T | 0.054 | 1.549 | 0.055 | 1248 |
| Phage-1051 | — | — | — | — | A | M | — | — | — | — | — | — | A | E | 0.056 | 1.123 | 0.058 | 1249 |
| Phage-1052 | L | — | — | — | A | M | — | D | — | — | — | — | G | P | 0.067 | 0.939 | 0.059 | 1250 |
| Phage-1053 | — | — | — | — | A | Q | — | G | — | — | I | — | V | T | 0.058 | 1.548 | 0.059 | 1251 |
| Phage-1054 | I | — | — | — | A | T | — | N | — | — | — | — | G | N | 0.055 | 2.674 | 0.056 | 1252 |
| Phage-1055 | — | — | — | — | A | T | — | G | — | — | — | — | F | T | 0.068 | 2.290 | 0.061 | 1253 |
| Phage-1056 | I | — | — | — | A | T | — | — | — | — | — | — | A | S | 0.057 | 2.083 | 0.056 | 1254 |
| Phage-1057 | W | T | — | — | A | T | S | M | — | R | Y | — | V | D | 0.150 | 1.312 | 0.226 | 1255 |
| Phage-1058 | W | K | — | — | A | T | S | M | — | R | Y | — | V | D | 0.130 | 2.690 | 0.194 | 1256 |
| Phage-1059 | — | — | — | — | A | V | — | E | — | — | I | — | R | H | 0.054 | 2.645 | 0.068 | 1257 |
| Phage-1060 | P | — | — | — | A | V | — | N | — | — | — | — | T | N | 0.055 | 2.628 | 0.058 | 1258 |
| Phage-1061 | — | — | — | — | A | V | — | K | — | — | — | — | L | E | 0.060 | 2.520 | 0.056 | 1259 |
| Phage-1062 | L | — | — | — | A | V | — | R | — | — | — | — | N | M | 0.059 | 2.380 | 0.062 | 1260 |
| Phage-1063 | — | — | — | — | A | V | — | K | — | — | — | — | S | I | 0.056 | 2.338 | 0.056 | 1261 |
| Phage-1064 | I | — | W | — | A | V | — | K | — | — | I | — | L | D | 0.067 | 2.277 | 0.075 | 1262 |
| Phage-1065 | — | — | — | — | A | V | — | G | — | — | — | — | Q | T | 0.065 | 2.139 | 0.075 | 1263 |
| Phage-1066 | I | — | — | — | A | V | — | K | — | — | — | — | A | R | 0.091 | 1.866 | 0.084 | 1264 |
| Phage-1067 | — | — | — | — | A | V | — | G | — | — | I | — | D | A | 0.056 | 1.853 | 0.056 | 1265 |
| Phage-1068 | L | V | — | — | A | V | — | D | — | — | — | — | S | N | 0.061 | 0.975 | 0.061 | 1266 |
| Phage-1069 | — | — | — | — | A | V | W | R | — | — | — | — | D | S | 0.058 | 0.154 | 0.060 | 1267 |
| Phage-1070 | I | — | — | — | A | V | — | N | — | — | — | — | G | S | 0.059 | 2.735 | 0.082 | 1268 |
| Phage-1071 | E | — | — | — | D | — | — | H | — | — | — | — | R | M | 0.099 | 1.583 | 0.064 | 1269 |
| Phage-1072 | — | — | — | — | D | L | — | R | — | — | I | — | L | L | 0.056 | 2.822 | 0.061 | 1270 |
| Phage-1073 | — | — | — | — | D | L | F | — | — | — | I | — | N | R | 0.067 | 1.993 | 0.077 | 1271 |
| Phage-1074 | — | — | — | — | D | L | — | D | — | — | — | — | S | T | 0.056 | 1.157 | 0.058 | 1272 |
| Phage-1075 | P | Y | — | — | D | M | — | N | — | — | — | — | V | T | 0.090 | 2.564 | 0.073 | 1273 |
| Phage-1076 | — | — | — | — | E | E | — | H | — | — | — | — | D | S | 0.056 | 1.112 | 0.057 | 1274 |
| Phage-1077 | I | — | — | — | E | — | — | R | — | — | — | — | S | R | 0.070 | 2.689 | 0.074 | 1275 |
| Phage-1078 | — | — | — | — | E | — | — | G | — | — | I | — | F | D | 0.055 | 2.696 | 0.055 | 1276 |
| Phage-1079 | I | — | — | — | E | — | — | — | — | — | — | — | I | — | 0.053 | 2.687 | 0.055 | 1277 |
| Phage-1080 | — | — | — | — | E | — | — | E | — | — | I | — | N | D | 0.054 | 2.634 | 0.060 | 1278 |
| Phage-1081 | P | N | — | — | E | — | — | R | — | — | — | — | N | V | 0.055 | 1.858 | 0.058 | 1279 |
| Phage-1082 | L | N | — | — | E | — | N | R | — | — | — | — | L | G | 0.060 | 0.701 | 0.064 | 1280 |
| Phage-1083 | I | — | — | — | E | K | — | Q | — | — | — | — | L | T | 0.056 | 1.625 | 0.056 | 1281 |
| Phage-1084 | F | — | — | — | E | K | — | K | — | — | I | — | I | P | 0.060 | 2.651 | 0.062 | 1282 |
| Phage-1085 | F | — | — | — | E | K | — | K | — | — | I | — | N | T | 0.057 | 2.236 | 0.057 | 1283 |
| Phage-1086 | I | — | — | — | E | L | — | Q | — | — | — | — | G | H | 0.055 | 2.700 | 0.057 | 1284 |
| Phage-1087 | — | — | — | — | E | L | — | K | — | — | — | — | F | K | 0.059 | 2.460 | 0.060 | 1285 |
| Phage-1088 | I | — | — | — | E | L | — | H | — | — | — | — | S | S | 0.057 | 2.767 | 0.062 | 1286 |
| Phage-1089 | — | — | — | — | E | L | — | K | — | — | — | — | M | G | 0.060 | 2.766 | 0.063 | 1287 |
| Phage-1090 | I | — | — | — | E | L | — | — | — | — | — | — | G | D | 0.068 | 2.530 | 0.092 | 1288 |
| Phage-1091 | — | — | — | — | E | L | — | H | — | — | — | — | D | — | 0.056 | 2.745 | 0.057 | 1289 |
| Phage-1092 | L | — | — | — | E | L | — | H | — | — | — | — | T | L | 0.076 | 2.684 | 0.065 | 1290 |
| Phage-1093 | — | — | — | — | E | L | — | — | — | — | — | — | Y | L | 0.060 | 2.753 | 0.058 | 1291 |
| Phage-1094 | I | — | — | — | E | L | — | H | — | — | — | — | K | W | 0.079 | 2.725 | 0.104 | 1292 |
| Phage-1095 | P | — | — | — | E | L | — | R | — | — | I | — | R | L | 0.071 | 2.364 | 0.073 | 1293 |
| Phage-1096 | — | — | — | — | E | L | — | R | — | — | — | — | M | I | 0.075 | 2.156 | 0.073 | 1294 |
| Phage-1097 | F | — | — | — | E | L | — | N | — | — | — | — | Q | S | 0.080 | 2.130 | 0.071 | 1295 |
| Phage-1098 | L | — | — | — | E | L | — | — | — | — | I | — | D | I | 0.056 | 2.093 | 0.064 | 1296 |
| Phage-1099 | — | — | — | — | E | L | — | E | — | — | I | — | F | K | 0.078 | 2.052 | 0.075 | 1297 |
| Phage-1100 | — | — | — | — | E | L | — | R | — | — | — | — | Y | K | 0.054 | 1.841 | 0.058 | 1298 |
| Phage-1101 | L | — | — | — | E | L | — | R | — | — | — | — | G | I | 0.062 | 1.329 | 0.056 | 1299 |
| Phage-1102 | — | — | — | — | E | L | — | R | — | — | — | — | N | G | 0.067 | 1.215 | 0.056 | 1300 |
| Phage-1103 | P | — | — | — | E | L | — | Q | — | — | — | — | D | R | 0.054 | 1.120 | 0.065 | 1301 |
| Phage-1104 | — | — | — | — | E | M | — | N | — | — | — | — | R | M | 0.057 | 2.726 | 0.055 | 1302 |
| Phage-1105 | L | — | — | — | E | M | — | G | — | — | — | — | K | V | 0.062 | 2.712 | 0.068 | 1303 |
| Phage-1106 | L | — | — | — | E | M | — | D | — | — | — | — | R | L | 0.056 | 1.989 | 0.059 | 1304 |
| Phage-1107 | L | — | — | — | E | M | — | N | — | — | — | — | K | D | 0.059 | 1.416 | 0.063 | 1305 |
| Phage-1108 | E | — | — | — | E | M | — | N | — | — | — | — | W | K | 0.055 | 1.334 | 0.057 | 1306 |
| Phage-1109 | — | — | — | — | E | M | — | R | — | — | — | — | A | S | 0.053 | 1.289 | 0.055 | 1307 |
| Phage-1110 | P | — | — | — | E | M | — | K | — | — | — | — | D | G | 0.054 | 1.244 | 0.055 | 1308 |
| Phage-1111 | — | — | — | — | E | V | — | H | — | — | — | — | Q | R | 0.058 | 2.621 | 0.057 | 1309 |
| Phage-1112 | — | — | — | — | E | V | — | H | — | — | — | — | D | R | 0.054 | 2.016 | 0.056 | 1310 |
| Phage-1113 | L | — | — | — | E | V | — | R | — | — | I | — | G | D | 0.062 | 0.064 | 0.052 | 1311 |
| Phage-1114 | — | — | — | — | F | — | — | — | — | — | — | — | T | E | 0.055 | 2.306 | 0.058 | 1312 |
| Phage-1115 | I | — | — | — | F | — | — | N | — | — | — | — | N | T | 0.259 | 2.747 | 0.192 | 1313 |
| Phage-1116 | L | — | W | — | F | — | — | N | — | — | — | — | Q | T | 0.158 | 2.720 | 0.155 | 1314 |
| Phage-1117 | — | — | — | — | F | L | — | — | — | — | — | — | S | T | 0.058 | 2.364 | 0.062 | 1315 |
| Phage-1118 | I | — | — | — | F | L | — | — | — | — | — | — | R | E | 0.061 | 2.181 | 0.059 | 1316 |
| Phage-1119 | I | — | — | — | F | L | — | R | — | — | — | — | S | S | 0.084 | 2.678 | 0.106 | 1317 |
| Phage-1120 | P | — | — | — | F | L | — | D | — | — | — | — | Y | E | 0.054 | 1.113 | 0.056 | 1318 |
| Phage-1121 | L | — | — | — | F | M | — | D | — | — | — | — | R | G | 0.056 | 2.678 | 0.060 | 1319 |
| Phage-1122 | — | — | — | — | F | M | F | H | — | — | — | — | A | E | 0.071 | 2.481 | 0.072 | 1320 |
| Phage-1123 | — | — | — | — | F | M | — | R | — | — | — | — | E | T | 0.065 | 1.765 | 0.058 | 1321 |
| Phage-1124 | E | — | — | — | F | M | — | T | — | — | — | — | — | N | 0.057 | 0.227 | 0.057 | 1322 |
| Phage-1125 | — | — | — | — | F | R | — | N | — | — | I | — | M | R | 0.065 | 2.265 | 0.070 | 1323 |
| Phage-1126 | — | — | — | — | F | R | — | G | — | — | — | — | G | T | 0.072 | 0.827 | 0.081 | 1324 |
| Phage-1127 | — | — | — | — | G | — | — | R | — | — | — | — | N | M | 0.059 | 2.526 | 0.062 | 1325 |
| Phage-1128 | — | — | — | — | G | — | — | R | — | — | — | — | M | T | 0.055 | 2.087 | 0.061 | 1326 |
| Phage-1129 | I | — | — | — | G | — | — | Q | — | — | I | — | — | N | 0.153 | 2.773 | 0.295 | 1327 |
| Phage-1130 | L | — | — | — | G | — | — | R | — | — | — | — | E | T | 0.060 | 2.310 | 0.059 | 1328 |
| Phage-1131 | — | — | — | — | G | L | — | N | — | — | — | — | A | S | 0.059 | 2.788 | 0.056 | 1329 |
| Phage-1132 | — | — | — | — | G | L | — | N | — | — | — | — | Q | E | 0.066 | 2.373 | 0.074 | 1330 |
| Phage-1133 | — | — | — | — | G | L | — | H | — | — | I | — | D | K | 0.083 | 2.723 | 0.081 | 1331 |
| Phage-1134 | — | — | — | — | G | L | — | N | — | — | I | — | A | T | 0.056 | 1.438 | 0.057 | 1332 |
| Phage-1135 | — | — | — | — | G | L | — | R | — | — | — | — | N | — | 0.055 | 0.872 | 0.056 | 1333 |
| Phage-1136 | E | — | — | — | G | M | — | H | — | — | — | — | D | I | 0.075 | 1.887 | 0.058 | 1334 |
| Phage-1137 | I | — | — | — | G | M | — | E | — | — | I | — | E | M | 0.054 | 1.827 | 0.062 | 1335 |
| Phage-1138 | E | — | — | — | G | M | — | H | — | — | I | — | S | G | 0.057 | 1.626 | 0.060 | 1336 |
| Phage-1139 | — | — | — | — | G | M | — | H | — | — | I | — | Y | K | 0.083 | 2.731 | 0.097 | 1337 |
| Phage-1140 | F | — | — | — | G | T | — | H | — | — | — | — | E | S | 0.067 | 2.212 | 0.061 | 1338 |
| Phage-1141 | I | — | — | — | G | V | — | K | — | — | — | — | T | L | 0.066 | 1.604 | 0.061 | 1339 |
| Phage-1142 | — | E | — | — | H | L | — | N | — | — | I | — | S | K | 0.059 | 2.849 | 0.061 | 1340 |
| Phage-1143 | — | — | — | — | H | M | — | R | — | — | — | — | D | M | 0.062 | 2.419 | 0.060 | 1341 |
| Phage-1144 | — | — | — | — | I | — | — | N | — | — | — | — | R | D | 0.075 | 2.649 | 0.074 | 1342 |
| Phage-1145 | I | — | — | — | I | L | — | D | — | — | — | — | E | M | 0.057 | 1.498 | 0.056 | 1343 |
| Phage-1146 | — | — | — | — | — | E | — | N | — | — | I | — | S | D | 0.071 | 2.476 | 0.070 | 1344 |
| Phage-1147 | I | — | — | — | — | E | — | — | — | — | I | — | L | N | 0.078 | 1.470 | 0.079 | 1345 |
| Phage-1148 | L | — | — | — | — | — | — | D | — | — | I | — | G | H | 0.076 | 2.670 | 0.084 | 1346 |
| Phage-1149 | — | — | — | — | — | — | — | — | — | — | — | — | S | N | 0.064 | 2.623 | 0.063 | 1347 |
| Phage-1150 | L | — | — | — | — | — | N | D | — | — | I | — | — | L | 0.063 | 2.436 | 0.078 | 1348 |
| Phage-1151 | — | — | — | — | — | — | — | E | — | — | I | — | R | M | 0.055 | 2.418 | 0.066 | 1349 |
| Phage-1152 | L | — | — | — | — | — | — | G | — | — | — | — | Y | T | 0.064 | 1.291 | 0.060 | 1350 |
| Phage-1153 | I | — | — | — | — | L | — | E | — | — | — | — | F | I | 0.079 | 2.696 | 0.090 | 1351 |
| Phage-1154 | P | — | — | — | — | L | N | K | — | — | — | — | I | — | 0.064 | 2.291 | 0.066 | 1352 |
| Phage-1155 | — | — | — | — | — | L | — | H | — | — | — | — | T | G | 0.062 | 2.216 | 0.074 | 1353 |
| Phage-1156 | — | — | — | — | — | L | — | K | — | — | — | — | E | G | 0.060 | 2.201 | 0.062 | 1354 |
| Phage-1157 | P | — | — | — | — | L | — | Q | — | — | I | — | V | R | 0.058 | 2.201 | 0.059 | 1355 |
| Phage-1158 | L | — | — | — | — | L | — | H | — | — | — | — | R | D | 0.065 | 2.118 | 0.068 | 1356 |
| Phage-1159 | — | E | — | — | — | L | — | Q | — | — | I | — | Q | S | 0.056 | 1.273 | 0.064 | 1357 |
| Phage-1160 | L | — | — | — | — | L | — | H | — | — | — | — | S | D | 0.071 | 1.062 | 0.075 | 1358 |
| Phage-1161 | — | — | — | — | — | M | — | A | — | — | — | — | K | L | 0.056 | 2.162 | 0.063 | 1359 |
| Phage-1162 | — | — | — | — | — | M | — | A | — | — | — | — | E | M | 0.081 | 2.518 | 0.071 | 1360 |
| Phage-1163 | — | — | — | — | — | M | F | A | — | — | — | — | N | T | 0.084 | 2.476 | 0.093 | 1361 |
| Phage-1164 | M | — | — | — | — | M | — | R | — | — | I | — | G | M | 0.067 | 2.340 | 0.072 | 1362 |
| Phage-1165 | — | — | — | — | — | M | — | D | — | — | — | — | T | N | 0.067 | 1.732 | 0.074 | 1363 |
| Phage-1166 | F | — | — | — | — | M | — | D | — | — | — | — | R | L | 0.089 | 1.728 | 0.092 | 1364 |
| Phage-1167 | I | N | — | — | — | M | — | E | — | — | I | — | S | T | 0.067 | 1.694 | 0.064 | 1365 |
| Phage-1168 | — | — | — | — | — | V | — | N | — | — | — | — | W | L | 0.119 | 2.605 | 0.143 | 1366 |
| Phage-1169 | I | — | — | — | — | V | F | A | — | — | — | — | T | A | 0.096 | 2.212 | 0.097 | 1367 |
| Phage-1170 | I | — | — | — | L | — | — | Q | — | — | I | — | V | N | 0.060 | 2.883 | 0.062 | 1368 |
| Phage-1171 | — | — | — | — | L | — | — | Q | — | — | — | — | N | S | 0.054 | 2.722 | 0.057 | 1369 |
| Phage-1172 | I | — | — | — | L | — | — | N | — | — | — | — | Y | T | 0.060 | 2.581 | 0.065 | 1370 |
| Phage-1173 | — | — | — | — | L | — | — | K | — | — | — | — | G | L | 0.055 | 2.117 | 0.062 | 1371 |
| Phage-1174 | I | — | — | — | L | — | — | E | — | — | — | — | T | A | 0.066 | 2.039 | 0.076 | 1372 |
| Phage-1175 | — | — | — | — | L | K | — | N | — | — | — | — | D | G | 0.078 | 0.436 | 0.073 | 1373 |
| Phage-1176 | L | — | — | — | L | L | — | D | — | — | — | — | Q | T | 0.055 | 2.588 | 0.057 | 1374 |
| Phage-1177 | I | — | — | — | L | L | — | K | — | — | — | — | E | L | 0.056 | 2.770 | 0.060 | 1375 |
| Phage-1178 | L | N | — | — | L | L | — | D | — | — | — | — | A | S | 0.072 | 1.096 | 0.079 | 1376 |
| Phage-1179 | — | — | — | — | L | L | — | E | — | — | — | — | N | L | 0.054 | 0.629 | 0.066 | 1377 |
| Phage-1180 | I | — | — | — | L | M | — | N | — | — | — | — | T | T | 0.061 | 2.759 | 0.061 | 1378 |
| Phage-1181 | — | — | — | — | L | M | — | Q | — | — | I | — | — | M | 0.057 | 2.774 | 0.062 | 1379 |
| Phage-1182 | I | — | — | — | L | M | — | H | — | — | — | — | Y | E | 0.058 | 2.766 | 0.068 | 1380 |
| Phage-1183 | I | — | — | — | L | M | — | N | — | — | — | — | M | S | 0.055 | 2.384 | 0.058 | 1381 |
| Phage-1184 | L | — | — | — | L | M | — | K | — | — | — | — | D | F | 0.082 | 2.134 | 0.073 | 1382 |
| Phage-1185 | I | — | — | — | L | M | — | — | — | — | — | — | T | A | 0.082 | 1.634 | 0.086 | 1383 |
| Phage-1186 | I | N | — | — | L | V | — | D | — | — | — | — | K | — | 0.061 | 2.403 | 0.067 | 1384 |
| Phage-1187 | F | — | — | — | L | V | — | — | — | — | — | — | N | S | 0.059 | 1.424 | 0.061 | 1385 |
| Phage-1188 | I | — | — | — | L | V | — | N | — | — | — | — | R | S | 0.053 | 0.659 | 0.063 | 1386 |
| Phage-1189 | L | — | — | — | L | W | — | K | — | — | — | — | E | — | 0.168 | 2.176 | 0.226 | 1387 |
| Phage-1190 | I | — | — | — | M | F | — | E | — | — | I | — | G | T | 0.062 | 2.678 | 0.058 | 1388 |
| Phage-1191 | L | V | — | — | M | F | N | N | — | — | — | — | Q | H | 0.054 | 0.062 | 0.054 | 1389 |
| Phage-1192 | M | — | — | — | M | — | — | N | — | — | — | — | A | — | 0.057 | 2.791 | 0.056 | 1390 |
| Phage-1193 | L | — | — | — | M | — | — | A | — | — | I | — | A | — | 0.057 | 2.522 | 0.070 | 1391 |
| Phage-1194 | I | — | — | — | M | — | — | R | — | — | — | — | Q | V | 0.091 | 2.729 | 0.089 | 1392 |
| Phage-1195 | L | — | — | — | M | — | — | K | — | — | I | — | K | G | 0.072 | 2.621 | 0.086 | 1393 |
| Phage-1196 | L | — | — | — | M | — | N | D | — | — | — | — | S | K | 0.055 | 1.578 | 0.057 | 1394 |
| Phage-1197 | — | — | — | — | M | L | — | K | — | — | I | — | K | T | 0.068 | 2.714 | 0.078 | 1395 |
| Phage-1198 | — | — | — | — | M | L | — | N | — | — | — | — | M | E | 0.105 | 2.751 | 0.056 | 1396 |
| Phage-1199 | I | — | — | — | M | L | — | Q | — | — | — | — | — | K | 0.081 | 2.496 | 0.090 | 1397 |
| Phage-1200 | I | — | — | — | M | L | — | A | — | — | — | — | N | S | 0.058 | 1.542 | 0.065 | 1398 |
| Phage-1201 | — | — | — | — | M | L | — | — | — | — | — | — | L | T | 0.059 | 0.073 | 0.061 | 1399 |
| Phage-1202 | I | — | — | — | M | L | — | N | — | — | I | — | A | E | 0.094 | 2.803 | 0.145 | 1400 |
| Phage-1203 | P | — | — | — | M | M | — | D | — | — | — | — | R | I | 0.117 | 2.260 | 0.059 | 1401 |
| Phage-1204 | F | V | — | — | N | — | L | A | M | R | N | — | V | S | 0.055 | 2.712 | 1.924 | 1402 |
| Phage-1205 | — | — | — | — | N | — | — | N | — | — | — | — | L | D | 0.054 | 2.166 | 0.060 | 1403 |
| Phage-1206 | — | — | — | — | N | L | — | N | — | — | — | — | Q | N | 0.083 | 2.488 | 0.063 | 1404 |
| Phage-1207 | Y | W | L | — | N | Q | G | K | — | L | A | — | E | — | 0.067 | 0.068 | 0.086 | 1405 |
| Phage-1208 | F | V | L | — | N | T | L | T | M | R | E | — | V | S | 0.065 | 1.426 | 2.552 | 1406 |
| Phage-1209 | I | — | — | — | N | V | — | D | — | — | — | — | R | T | 0.055 | 2.663 | 0.061 | 1407 |
| Phage-1210 | — | T | — | — | P | T | S | Q | — | R | Y | — | I | I | 0.063 | 0.102 | 0.090 | 1408 |
| Phage-1211 | I | — | — | — | Q | E | — | N | — | — | — | — | V | N | 0.056 | 2.141 | 0.058 | 1409 |
| Phage-1212 | — | — | — | — | Q | — | — | — | — | — | — | — | N | W | 0.060 | 2.336 | 0.062 | 1410 |
| Phage-1213 | — | — | — | — | Q | — | — | K | — | — | — | — | E | — | 0.060 | 2.583 | 0.060 | 1411 |
| Phage-1214 | — | — | — | — | Q | — | — | N | — | — | — | — | T | D | 0.059 | 2.573 | 0.057 | 1412 |
| Phage-1215 | F | — | — | — | Q | — | — | N | — | — | I | — | G | R | 0.081 | 1.795 | 0.093 | 1413 |
| Phage-1216 | — | — | — | — | Q | — | — | A | — | — | — | — | G | N | 0.058 | 1.796 | 0.065 | 1414 |
| Phage-1217 | N | E | Q | — | Q | — | — | I | M | A | — | — | R | A | 0.074 | 0.065 | 0.074 | 1415 |
| Phage-1218 | — | — | — | — | Q | L | — | H | — | — | — | — | N | A | 0.056 | 2.830 | 0.062 | 1416 |
| Phage-1219 | P | — | — | — | Q | L | N | N | — | — | — | — | A | G | 0.062 | 2.428 | 0.057 | 1417 |
| Phage-1220 | — | — | — | — | Q | L | — | A | — | — | — | — | G | T | 0.056 | 2.428 | 0.057 | 1418 |
| Phage-1221 | I | — | — | — | Q | L | — | — | — | — | — | — | V | N | 0.062 | 2.046 | 0.058 | 1419 |
| Phage-1222 | L | — | — | — | Q | L | — | R | — | — | — | — | E | V | 0.060 | 1.887 | 0.059 | 1420 |
| Phage-1223 | — | — | — | — | Q | L | — | R | — | — | — | — | G | N | 0.067 | 1.583 | 0.062 | 1421 |
| Phage-1224 | — | — | W | — | Q | L | — | N | — | — | — | — | R | E | 0.060 | 0.211 | 0.074 | 1422 |
| Phage-1225 | — | — | — | — | Q | M | — | D | — | — | I | — | M | A | 0.063 | 2.464 | 0.063 | 1423 |
| Phage-1226 | I | — | — | — | Q | M | — | — | — | — | — | — | G | — | 0.069 | 2.089 | 0.069 | 1424 |
| Phage-1227 | — | — | — | — | Q | M | — | M | — | — | — | — | L | S | 0.243 | 2.619 | 0.257 | 1425 |
| Phage-1228 | I | — | — | — | Q | V | — | — | — | — | — | — | E | — | 0.054 | 2.449 | 0.055 | 1426 |
| Phage-1229 | M | — | — | — | Q | V | — | A | — | — | — | — | N | M | 0.054 | 2.130 | 0.057 | 1427 |
| Phage-1230 | — | — | — | — | Q | V | — | N | — | — | — | — | L | S | 0.057 | 1.133 | 0.057 | 1428 |
| Phage-1231 | L | — | — | — | Q | V | — | N | — | — | I | — | A | W | 0.065 | 0.720 | 0.074 | 1429 |
| Phage-1232 | L | E | S | — | Q | V | — | E | — | H | G | — | G | A | 0.066 | 0.059 | 0.059 | 1430 |
| Phage-1233 | I | — | — | — | R | E | — | N | — | — | — | — | S | W | 0.072 | 1.660 | 0.076 | 1431 |
| Phage-1234 | — | — | — | — | R | F | — | D | — | — | — | — | D | T | 0.055 | 1.646 | 0.063 | 1432 |
| Phage-1235 | L | — | — | — | R | — | — | R | — | — | — | — | E | T | 0.070 | 1.819 | 0.063 | 1433 |
| Phage-1236 | — | — | — | — | R | — | — | H | — | — | — | — | E | D | 0.065 | 2.723 | 0.062 | 1434 |
| Phage-1237 | — | — | — | — | R | — | — | Q | — | — | — | — | G | S | 0.053 | 0.345 | 0.054 | 1435 |
| Phage-1238 | — | — | — | — | R | — | — | N | — | — | — | — | I | N | 0.078 | 2.287 | 0.079 | 1436 |
| Phage-1239 | — | — | — | — | R | — | — | Q | — | — | — | — | T | Y | 0.059 | 1.675 | 0.061 | 1437 |
| Phage-1240 | — | — | — | — | R | — | — | — | — | — | — | — | T | L | 0.059 | 1.442 | 0.061 | 1438 |
| Phage-1241 | L | — | — | — | R | — | — | N | — | — | — | — | A | R | 0.090 | 1.189 | 0.092 | 1439 |
| Phage-1242 | L | — | — | — | R | — | N | D | — | — | — | — | Q | S | 0.061 | 0.899 | 0.056 | 1440 |
| Phage-1243 | — | — | — | — | R | L | — | H | — | — | — | — | G | D | 0.076 | 2.276 | 0.076 | 1441 |
| Phage-1244 | I | — | — | — | R | L | — | D | — | — | — | — | G | L | 0.067 | 2.435 | 0.062 | 1442 |
| Phage-1245 | — | — | — | — | R | L | — | D | — | — | — | — | Q | D | 0.054 | 1.729 | 0.056 | 1443 |
| Phage-1246 | — | — | W | — | R | L | — | Q | — | — | — | — | Y | — | 0.255 | 2.541 | 0.334 | 1444 |
| Phage-1247 | I | — | — | — | R | L | — | N | — | — | I | — | P | G | 0.072 | 2.698 | 0.089 | 1445 |
| Phage-1248 | L | — | — | — | R | L | — | K | — | — | I | — | S | V | 0.226 | 2.666 | 0.267 | 1446 |
| Phage-1249 | L | — | — | — | R | L | — | N | — | — | — | — | S | M | 0.107 | 2.576 | 0.124 | 1447 |
| Phage-1250 | L | — | — | — | R | L | — | N | — | — | — | — | G | V | 0.081 | 2.527 | 0.073 | 1448 |
| Phage-1251 | — | — | — | — | R | L | — | N | — | — | — | — | S | V | 0.056 | 2.276 | 0.059 | 1449 |
| Phage-1252 | — | — | — | — | R | L | — | — | — | — | — | — | Q | E | 0.059 | 2.032 | 0.059 | 1450 |
| Phage-1253 | — | — | — | — | R | L | — | T | — | — | I | — | Q | S | 0.074 | 1.979 | 0.102 | 1451 |
| Phage-1254 | P | — | — | — | R | L | — | D | — | — | — | — | A | K | 0.065 | 1.823 | 0.057 | 1452 |
| Phage-1255 | L | — | — | — | R | L | N | D | — | — | — | — | Q | T | 0.056 | 1.551 | 0.070 | 1453 |
| Phage-1256 | — | — | — | — | R | L | — | D | — | — | — | — | I | E | 0.078 | 0.978 | 0.074 | 1454 |
| Phage-1257 | I | — | W | — | R | M | — | E | — | — | — | — | K | H | 0.080 | 1.130 | 0.098 | 1455 |
| Phage-1258 | — | — | — | — | R | V | — | N | — | — | — | — | Q | V | 0.067 | 2.726 | 0.069 | 1456 |
| Phage-1259 | I | — | — | — | R | V | — | E | — | — | I | — | Y | G | 0.056 | 2.433 | 0.058 | 1457 |
| Phage-1260 | I | — | — | — | R | V | — | D | — | — | — | — | N | R | 0.063 | 2.155 | 0.056 | 1458 |
| Phage-1261 | I | — | — | — | R | V | — | D | — | — | — | — | N | W | 0.084 | 2.489 | 0.079 | 1459 |
| Phage-1262 | D | W | — | — | R | V | — | — | — | — | I | — | Q | D | 0.316 | 2.348 | 0.404 | 1460 |
| Phage-1263 | I | — | — | — | R | V | — | — | — | — | — | — | G | T | 0.059 | 1.879 | 0.055 | 1461 |
| Phage-1264 | — | — | W | — | R | Y | — | D | — | — | — | — | S | T | 0.075 | 0.652 | 0.095 | 1462 |
| Phage-1265 | — | — | — | — | S | — | — | H | — | — | — | — | Y | V | 0.067 | 2.862 | 0.078 | 1463 |
| Phage-1266 | — | — | — | — | S | — | — | R | — | — | — | — | T | G | 0.065 | 1.384 | 0.068 | 1464 |
| Phage-1267 | I | — | — | — | S | — | — | — | — | — | — | — | R | H | 0.072 | 2.321 | 0.105 | 1465 |
| Phage-1268 | L | — | — | — | S | — | N | D | — | — | — | — | N | V | 0.061 | 1.436 | 0.059 | 1466 |
| Phage-1269 | I | — | W | — | S | L | — | A | — | — | — | — | E | L | 0.066 | 2.508 | 0.069 | 1467 |
| Phage-1270 | E | — | — | — | S | L | — | K | — | — | I | — | N | S | 0.073 | 2.525 | 0.089 | 1468 |
| Phage-1271 | — | — | — | — | S | L | — | D | — | — | — | — | N | — | 0.055 | 2.115 | 0.053 | 1469 |
| Phage-1272 | F | — | — | — | S | L | — | K | — | — | — | — | E | T | 0.059 | 1.805 | 0.068 | 1470 |
| Phage-1273 | — | — | — | — | S | L | — | D | — | — | — | — | S | G | 0.053 | 2.153 | 0.057 | 1471 |
| Phage-1274 | — | — | — | — | S | L | — | N | — | — | — | — | Q | N | 0.059 | 2.725 | 0.063 | 1472 |
| Phage-1275 | — | — | — | — | S | L | — | N | — | — | — | — | K | M | 0.059 | 2.714 | 0.064 | 1473 |
| Phage-1276 | I | — | — | — | S | L | — | — | — | — | — | — | M | S | 0.056 | 2.694 | 0.056 | 1474 |
| Phage-1277 | — | — | — | — | S | L | — | N | — | — | — | — | M | R | 0.082 | 2.694 | 0.090 | 1475 |
| Phage-1278 | — | — | — | — | S | L | — | H | — | — | — | — | M | — | 0.058 | 2.653 | 0.062 | 1476 |
| Phage-1279 | — | — | — | — | S | L | — | Q | — | — | — | — | D | V | 0.080 | 2.335 | 0.077 | 1477 |
| Phage-1280 | I | — | — | — | S | L | — | K | — | — | I | — | G | A | 0.056 | 2.197 | 0.060 | 1478 |
| Phage-1281 | M | — | — | — | S | L | — | N | — | — | — | — | R | L | 0.071 | 1.469 | 0.067 | 1479 |
| Phage-1282 | L | — | — | — | S | L | — | A | — | — | I | — | A | K | 0.063 | 1.323 | 0.067 | 1480 |
| Phage-1283 | — | — | — | — | S | L | — | D | — | — | — | — | L | S | 0.062 | 1.281 | 0.060 | 1481 |
| Phage-1284 | I | — | — | — | S | L | — | K | — | — | I | — | G | G | 0.065 | 2.760 | 0.086 | 1482 |
| Phage-1285 | — | — | — | — | S | M | — | N | — | — | — | — | A | R | 0.054 | 2.428 | 0.067 | 1483 |
| Phage-1286 | — | — | — | — | S | M | — | N | — | — | — | — | V | — | 0.059 | 2.611 | 0.061 | 1484 |
| Phage-1287 | L | — | — | — | S | M | — | H | — | — | — | — | G | D | 0.057 | 2.570 | 0.056 | 1485 |
| Phage-1288 | L | — | — | — | S | M | — | N | — | — | — | — | R | E | 0.057 | 2.200 | 0.062 | 1486 |
| Phage-1289 | — | — | — | — | S | M | — | Q | — | — | — | — | D | G | 0.074 | 1.991 | 0.073 | 1487 |
| Phage-1290 | — | — | — | — | S | M | — | M | — | — | — | — | Y | — | 0.057 | 1.808 | 0.072 | 1488 |
| Phage-1291 | L | — | — | — | S | M | — | D | — | — | — | — | A | R | 0.058 | 0.939 | 0.059 | 1489 |
| Phage-1292 | P | — | — | — | S | M | — | Q | — | — | — | — | Q | E | 0.081 | 0.409 | 0.064 | 1490 |
| Phage-1293 | — | — | — | — | S | M | — | A | — | — | — | — | K | T | 0.053 | 0.327 | 0.065 | 1491 |
| Phage-1294 | I | — | — | — | S | V | — | N | — | — | — | — | D | P | 0.081 | 2.329 | 0.069 | 1492 |
| Phage-1295 | M | — | — | — | S | V | — | Q | — | — | I | — | G | S | 0.070 | 2.495 | 0.089 | 1493 |
| Phage-1296 | — | — | — | — | S | V | — | Q | — | — | I | — | Y | K | 0.060 | 1.925 | 0.065 | 1494 |
| Phage-1297 | — | — | W | — | S | V | — | D | — | — | — | — | R | — | 0.057 | 0.453 | 0.059 | 1495 |
| Phage-1298 | L | — | — | — | S | Y | — | D | — | — | — | — | R | T | 0.058 | 2.921 | 0.065 | 1496 |
| Phage-1299 | L | — | — | — | T | F | — | D | — | — | — | — | I | G | 0.057 | 0.658 | 0.058 | 1497 |
| Phage-1300 | — | — | — | — | T | — | — | H | — | — | — | — | F | T | 0.062 | 2.800 | 0.064 | 1498 |
| Phage-1301 | L | — | — | — | T | — | — | N | — | — | I | — | N | R | 0.060 | 0.955 | 0.058 | 1499 |
| Phage-1302 | L | — | — | — | T | L | — | K | — | — | — | — | K | I | 0.064 | 1.606 | 0.066 | 1500 |
| Phage-1303 | — | — | — | — | T | L | — | H | — | — | — | — | T | V | 0.069 | 1.880 | 0.088 | 1501 |
| Phage-1304 | I | — | — | — | T | M | — | H | — | — | I | — | D | T | 0.057 | 2.853 | 0.066 | 1502 |
| Phage-1305 | L | — | — | — | T | M | — | D | — | — | — | — | G | H | 0.055 | 2.583 | 0.065 | 1503 |
| Phage-1306 | I | — | — | — | T | M | — | Q | — | — | I | — | D | T | 0.076 | 2.761 | 0.071 | 1504 |
| Phage-1307 | — | — | — | — | T | V | — | N | — | — | — | — | Q | T | 0.061 | 2.843 | 0.059 | 1505 |
| Phage-1308 | I | — | — | — | T | V | — | K | — | — | — | — | Q | I | 0.059 | 2.833 | 0.074 | 1506 |
| Phage-1309 | — | — | — | — | T | V | — | R | — | — | I | — | S | N | 0.059 | 2.799 | 0.081 | 1507 |
| Phage-1310 | F | — | — | — | T | V | — | — | — | — | — | — | R | — | 0.058 | 0.385 | 0.062 | 1508 |
| Phage-1311 | L | — | L | — | T | V | Q | — | G | — | — | — | R | S | 0.055 | 0.056 | 0.056 | 1509 |
| Phage-1312 | I | — | — | — | V | — | — | H | — | — | — | — | R | R | 0.058 | 2.750 | 0.064 | 1510 |
| Phage-1313 | I | — | — | — | V | L | — | D | — | — | — | — | Q | G | 0.054 | 2.606 | 0.060 | 1511 |
| Phage-1314 | L | — | — | — | V | L | — | R | — | — | I | — | S | T | 0.072 | 2.459 | 0.072 | 1512 |
| Phage-1315 | — | — | — | — | V | V | — | Q | — | — | I | — | G | — | 0.056 | 2.367 | 0.074 | 1513 |
| Phage-1316 | — | — | — | — | W | — | — | R | — | — | — | — | L | A | 0.467 | 2.853 | 0.514 | 1514 |
| Phage-1317 | — | — | — | — | W | — | — | E | — | — | I | — | L | N | 0.059 | 2.146 | 0.070 | 1515 |
| Phage-1318 | T | — | — | — | W | — | — | D | — | — | — | — | — | — | 0.055 | 0.474 | 0.067 | 1516 |
| Phage-1319 | — | — | — | — | W | L | — | K | — | — | — | — | F | — | 0.288 | 2.659 | 0.327 | 1517 |
| Phage-1320 | — | — | — | — | W | L | — | K | — | — | — | — | E | N | 0.065 | 1.993 | 0.062 | 1518 |
| Phage-1321 | E | — | — | — | W | L | — | K | — | — | — | — | T | L | 0.060 | 1.750 | 0.057 | 1519 |
| Phage-1322 | — | — | — | — | W | L | — | E | — | — | — | — | Q | N | 0.055 | 1.633 | 0.060 | 1520 |
| Phage-1323 | — | — | — | — | W | M | — | D | — | — | — | — | R | E | 0.055 | 2.656 | 0.064 | 1521 |
| Phage-1324 | E | — | — | — | W | M | — | A | — | — | — | — | G | I | 0.059 | 1.958 | 0.062 | 1522 |
| Phage-1325 | — | — | — | — | Y | E | — | D | — | — | — | — | — | T | 0.057 | 0.797 | 0.059 | 1523 |
| Phage-1326 | P | — | — | — | Y | — | N | N | — | — | — | — | V | T | 0.057 | 2.321 | 0.057 | 1524 |
| Phage-1327 | P | — | — | — | Y | — | — | K | — | — | I | — | T | E | 0.065 | 2.157 | 0.064 | 1525 |
| Phage-1328 | I | — | — | — | Y | — | — | D | — | — | — | — | Y | T | 0.056 | 2.157 | 0.068 | 1526 |
| Phage-1329 | L | — | — | — | Y | — | N | K | — | — | — | — | W | E | 0.057 | 1.901 | 0.076 | 1527 |
| Phage-1330 | — | — | — | — | Y | L | — | — | — | — | I | — | T | K | 0.063 | 2.858 | 0.062 | 1528 |
| Phage-1331 | — | — | — | — | Y | L | F | N | — | — | — | — | W | E | 0.150 | 2.833 | 0.134 | 1529 |
| Phage-1332 | — | — | — | — | Y | L | — | D | — | — | — | — | N | S | 0.053 | 2.601 | 0.064 | 1530 |
| Phage-1333 | — | — | — | — | Y | L | — | — | — | — | — | — | V | — | 0.088 | 2.495 | 0.073 | 1531 |
| Phage-1334 | — | — | — | — | Y | L | F | D | — | — | — | — | P | N | 0.073 | 2.620 | 0.078 | 1532 |
| Phage-1335 | F | — | — | — | Y | L | — | D | — | — | — | — | W | G | 0.066 | 2.600 | 0.075 | 1533 |
| Phage-1336 | M | — | — | — | Y | L | — | R | — | — | I | — | G | M | 0.071 | 1.341 | 0.075 | 1534 |
| Phage-1337 | P | — | — | — | Y | M | — | N | — | — | — | — | V | T | 0.070 | 2.778 | 0.072 | 1535 |
| Phage-1338 | P | — | — | — | Y | M | — | G | — | — | I | — | D | S | 0.055 | 2.835 | 0.056 | 1536 |
| Phage-1339 | — | — | — | — | Y | M | — | R | — | — | — | — | L | N | 0.063 | 2.740 | 0.076 | 1537 |
| Phage-1340 | P | — | — | — | Y | M | — | R | — | — | — | — | M | S | 0.059 | 2.646 | 0.063 | 1538 |
| Phage-1341 | P | — | — | — | Y | M | — | G | — | — | I | — | D | T | 0.053 | 2.742 | 0.075 | 1539 |
| Phage-1342 | — | — | — | — | Y | M | — | G | — | — | — | — | R | M | 0.055 | 2.664 | 0.057 | 1540 |
| Phage-1343 | — | — | — | — | Y | M | — | N | — | — | — | — | V | P | 0.055 | 2.663 | 0.067 | 1541 |
| Phage-1344 | — | — | — | — | Y | M | — | G | — | — | — | — | A | A | 0.066 | 2.624 | 0.057 | 1542 |
| Phage-1345 | L | — | — | — | Y | M | — | G | — | — | I | — | A | S | 0.056 | 2.598 | 0.060 | 1543 |
| Phage-1346 | — | — | — | — | Y | M | — | D | — | — | — | — | V | R | 0.080 | 2.573 | 0.061 | 1544 |
| Phage-1347 | — | — | — | — | Y | M | — | A | — | — | — | — | M | A | 0.056 | 2.563 | 0.058 | 1545 |
| Phage-1348 | E | — | — | — | Y | M | — | R | — | — | — | — | Y | R | 0.058 | 2.441 | 0.058 | 1546 |
| Phage-1349 | — | — | — | — | Y | M | — | G | — | — | — | — | V | — | 0.071 | 2.041 | 0.058 | 1547 |
| Phage-1350 | A | — | — | — | Y | M | — | D | — | — | — | — | S | K | 0.054 | 1.289 | 0.068 | 1548 |
| Phage-1351 | E | — | — | — | Y | R | — | N | — | — | — | — | K | L | 0.054 | 2.581 | 0.056 | 1549 |
| Phage-1352 | F | — | — | — | Y | R | — | N | — | — | I | — | T | K | 0.274 | 2.176 | 0.336 | 1550 |
| Phage-1353 | — | — | — | — | Y | R | — | N | — | — | L | — | R | V | 0.095 | 1.623 | 0.127 | 1551 |
| Phage-1354 | I | — | — | — | Y | R | — | G | — | — | — | — | G | T | 0.060 | 1.592 | 0.066 | 1552 |
| Phage-1355 | I | — | — | — | Y | R | — | G | — | — | — | — | Q | T | 0.058 | 1.535 | 0.059 | 1553 |
| Phage-1356 | I | — | — | — | Y | R | — | G | — | — | — | — | Y | T | 0.082 | 1.336 | 0.074 | 1554 |
| Phage-1357 | — | — | — | — | Y | V | — | D | — | — | — | — | S | S | 0.055 | 2.162 | 0.058 | 1555 |
| Phage-1358 | I | — | — | — | Q | L | — | H | — | — | — | — | R | A | 0.110 | 2.717 | 0.699 | 1556 |
| Phage-1359 | — | — | — | — | A | L | — | N | — | — | — | — | A | I | 0.070 | 2.762 | 0.426 | 1557 |
| Phage-1360 | F | — | — | — | F | M | — | H | — | — | — | — | K | D | 0.225 | 2.870 | 0.398 | 1558 |
| Phage-1361 | I | — | — | — | E | — | — | R | — | — | — | — | Y | D | 0.068 | 2.837 | 0.134 | 1559 |
| Phage-1362 | — | — | — | — | T | M | — | E | — | — | I | — | E | K | 0.116 | 2.415 | 0.144 | 1560 |
| Phage-1363 | — | — | W | — | Y | M | — | A | — | — | — | — | S | — | 0.079 | 2.820 | 0.161 | 1561 |
| Phage-1364 | I | — | — | — | F | — | — | R | — | — | — | — | S | Y | 0.432 | 2.722 | 0.527 | 1562 |
| Phage-1365 | I | — | — | — | T | L | — | N | — | — | I | — | D | G | 0.093 | 2.747 | 0.121 | 1563 |
| Phage-1366 | L | — | — | — | A | — | — | D | — | — | — | — | E | — | 0.095 | 2.706 | 0.088 | 1564 |
| Phage-1367 | L | — | — | — | A | L | — | D | — | — | — | — | K | E | 0.178 | 1.820 | 0.118 | 1565 |
| Phage-1368 | L | — | — | — | E | V | — | R | — | — | I | — | G | V | 0.064 | 2.833 | 0.357 | 1566 |
| Phage-1369 | L | — | — | — | — | — | — | R | — | — | — | — | T | P | 0.146 | 2.734 | 0.184 | 1567 |
| Phage-1370 | L | — | — | — | — | L | — | K | — | — | I | — | T | T | 0.121 | 2.772 | 0.193 | 1568 |
| Phage-1371 | L | — | — | — | R | V | — | K | — | — | — | — | D | M | 0.071 | 2.638 | 0.088 | 1569 |
| Phage-1372 | L | — | — | — | S | L | — | D | — | — | I | — | R | S | 0.069 | 2.725 | 0.070 | 1570 |
| Phage-1373 | L | — | — | — | S | M | — | N | — | — | — | — | S | V | 0.098 | 2.683 | 0.083 | 1571 |
| Phage-1374 | P | — | — | — | A | M | — | — | — | — | — | — | V | K | 0.071 | 2.718 | 0.079 | 1572 |
| Phage-1375 | P | — | — | — | — | L | — | K | — | — | — | — | I | — | 0.072 | 2.825 | 0.199 | 1573 |
| Phage-1376 | T | — | — | — | L | L | — | N | — | — | — | — | Q | H | 0.098 | 2.706 | 0.142 | 1574 |
| Phage-1377 | — | — | — | — | A | F | — | D | — | — | — | — | D | M | 0.080 | 2.740 | 0.087 | 1575 |
| Phage-1378 | — | — | — | — | Y | H | — | N | — | — | — | — | N | F | 0.077 | 2.710 | 0.099 | 1576 |
| Phage-1379 | — | — | — | — | Y | L | F | N | — | — | — | — | G | K | 0.130 | 2.757 | 0.191 | 1577 |
| Phage-1380 | A | — | — | — | A | M | — | H | — | — | M | — | D | — | 0.085 | 2.757 | 0.097 | 1578 |
| Phage-1381 | A | — | — | — | Y | L | — | H | — | — | — | — | Y | V | 0.084 | 2.322 | 0.154 | 1579 |
| Phage-1382 | D | E | — | — | I | Q | — | R | — | — | I | — | E | S | 0.060 | 2.135 | 0.094 | 1580 |
| Phage-1383 | E | — | — | — | A | L | — | R | — | — | — | — | L | S | 0.074 | 1.190 | 0.096 | 1581 |
| Phage-1384 | E | — | — | — | E | W | — | H | — | — | — | — | A | N | 0.061 | 2.728 | 0.991 | 1582 |
| Phage-1385 | E | — | — | — | F | F | — | N | — | — | — | — | A | A | 0.064 | 2.737 | 0.241 | 1583 |
| Phage-1386 | E | — | — | — | L | — | — | D | — | — | — | — | V | — | 0.088 | 2.104 | 0.078 | 1584 |
| Phage-1387 | E | — | — | — | M | L | — | R | — | — | — | — | V | S | 0.073 | 2.730 | 0.816 | 1585 |
| Phage-1388 | E | N | — | — | A | M | — | N | — | — | — | — | N | L | 0.077 | 2.714 | 0.253 | 1586 |
| Phage-1389 | F | — | — | — | A | M | — | M | — | — | — | — | L | M | 0.344 | 2.703 | 0.548 | 1587 |
| Phage-1390 | F | — | — | — | D | V | — | Y | — | — | — | — | — | A | 0.089 | 2.741 | 0.089 | 1588 |
| Phage-1391 | F | — | — | — | E | V | — | K | — | — | — | — | Y | L | 0.113 | 0.642 | 0.126 | 1589 |
| Phage-1392 | F | — | — | — | S | K | — | K | — | — | I | — | D | G | 0.079 | 1.162 | 0.208 | 1590 |
| Phage-1393 | G | N | — | — | A | T | — | A | — | — | I | — | V | N | 0.121 | 2.710 | 0.594 | 1591 |
| Phage-1394 | I | — | — | — | A | — | — | Q | — | — | — | — | W | N | 0.080 | 2.691 | 0.080 | 1592 |
| Phage-1395 | I | — | — | — | A | M | — | H | — | — | I | — | D | K | 0.075 | 2.806 | 0.156 | 1593 |
| Phage-1396 | I | — | — | — | A | M | — | Q | — | — | — | — | K | S | 0.103 | 2.733 | 0.086 | 1594 |
| Phage-1397 | I | — | — | — | A | M | — | R | — | — | I | — | N | I | 0.076 | 2.735 | 0.919 | 1595 |
| Phage-1398 | I | — | — | — | A | V | — | K | — | — | M | — | Q | I | 0.065 | 1.530 | 0.105 | 1596 |
| Phage-1399 | I | — | — | — | A | V | — | — | — | — | — | — | Y | L | 0.075 | 2.704 | 0.120 | 1597 |
| Phage-1400 | I | — | — | — | D | — | — | R | — | — | — | — | S | — | 0.066 | 2.751 | 0.068 | 1598 |
| Phage-1401 | I | — | — | — | D | M | — | Q | — | — | I | — | D | T | 0.153 | 2.728 | 0.292 | 1599 |
| Phage-1402 | I | — | — | — | E | — | F | — | — | — | — | — | R | P | 0.084 | 2.823 | 0.267 | 1600 |
| Phage-1403 | I | — | — | — | F | L | — | Q | — | — | I | — | D | T | 0.073 | 2.718 | 0.073 | 1601 |
| Phage-1404 | I | — | — | — | I | — | — | R | — | — | — | — | L | V | 0.118 | 0.627 | 0.127 | 1602 |
| Phage-1405 | I | — | — | — | — | — | — | V | — | — | I | — | N | S | 0.093 | 2.837 | 0.317 | 1603 |
| Phage-1406 | I | — | — | — | — | V | — | K | — | — | — | — | M | N | 0.086 | 2.478 | 0.126 | 1604 |
| Phage-1407 | I | — | — | — | M | L | — | H | — | — | I | — | D | G | 0.121 | 2.738 | 0.144 | 1605 |
| Phage-1408 | I | — | — | — | M | M | F | D | — | — | I | — | T | K | 0.116 | 2.695 | 0.117 | 1606 |
| Phage-1409 | I | — | — | — | P | M | — | Q | — | T | I | — | I | T | 0.080 | 2.705 | 0.081 | 1607 |
| Phage-1410 | I | — | — | — | Q | M | — | R | — | — | — | — | Y | G | 0.132 | 2.671 | 0.167 | 1608 |
| Phage-1411 | I | — | — | — | R | M | — | K | — | — | — | — | Y | G | 0.284 | 2.830 | 0.431 | 1609 |
| Phage-1412 | I | — | — | — | R | V | — | D | — | — | — | — | S | L | 0.072 | 2.704 | 0.128 | 1610 |
| Phage-1413 | I | — | — | — | S | — | — | W | — | — | — | — | Q | E | 0.160 | 2.731 | 0.177 | 1611 |
| Phage-1414 | I | — | — | — | S | L | — | E | — | — | — | — | W | S | 1.574 | 2.721 | 1.471 | 1612 |
| Phage-1415 | I | — | — | — | S | L | — | T | — | — | — | — | Q | T | 0.094 | 2.739 | 0.074 | 1613 |
| Phage-1416 | I | — | — | — | S | V | — | Y | — | — | — | — | N | S | 0.087 | 0.808 | 0.141 | 1614 |
| Phage-1417 | I | — | — | — | V | L | — | E | — | — | — | — | G | T | 0.061 | 2.687 | 0.122 | 1615 |
| Phage-1418 | I | — | — | — | W | M | — | Q | — | — | — | — | D | Y | 0.106 | 2.690 | 0.212 | 1616 |
| Phage-1419 | I | — | — | — | W | Q | — | G | — | — | — | — | Y | G | 0.126 | 2.666 | 0.118 | 1617 |
| Phage-1420 | I | — | — | — | Y | L | — | Q | — | — | I | — | T | K | 0.064 | 2.748 | 0.067 | 1618 |
| Phage-1421 | I | N | — | — | R | — | — | D | — | — | — | — | — | D | 0.098 | 0.552 | 0.122 | 1619 |
| Phage-1422 | K | — | — | — | Y | W | — | Q | — | — | I | — | G | D | 0.071 | 2.855 | 0.219 | 1620 |
| Phage-1423 | L | — | — | — | A | L | N | R | — | — | — | — | Q | A | 0.127 | 2.426 | 0.169 | 1621 |
| Phage-1424 | L | — | — | — | A | L | — | R | — | — | — | — | Q | A | 0.082 | 2.484 | 0.175 | 1622 |
| Phage-1425 | L | — | — | — | A | L | — | R | — | — | — | — | S | V | 0.109 | 1.974 | 0.135 | 1623 |
| Phage-1426 | L | — | — | — | A | M | — | K | — | — | — | — | R | S | 0.113 | 2.763 | 0.159 | 1624 |
| Phage-1427 | L | — | — | — | E | L | — | R | — | — | — | — | W | M | 0.250 | 1.784 | 0.314 | 1625 |
| Phage-1428 | L | — | — | — | E | M | — | Q | — | — | — | — | R | H | 0.069 | 2.685 | 0.089 | 1626 |
| Phage-1429 | L | — | — | — | E | R | — | A | — | — | I | — | Y | G | 0.080 | 0.553 | 0.091 | 1627 |
| Phage-1430 | L | — | — | — | — | — | N | D | — | — | I | — | G | H | 0.068 | 2.712 | 0.068 | 1628 |
| Phage-1431 | L | — | — | — | — | — | N | G | — | — | — | — | Y | T | 0.087 | 2.817 | 0.134 | 1629 |
| Phage-1432 | L | — | — | — | — | — | — | D | — | — | I | — | D | H | 0.088 | 2.692 | 0.090 | 1630 |
| Phage-1433 | L | — | — | — | — | L | — | R | — | — | — | — | I | — | 0.205 | 2.503 | 0.287 | 1631 |
| Phage-1434 | L | — | — | — | — | V | — | — | — | — | I | — | R | V | 0.218 | 1.602 | 0.240 | 1632 |
| Phage-1435 | L | — | — | — | L | M | — | N | — | — | — | — | S | H | 0.143 | 2.484 | 0.151 | 1633 |
| Phage-1436 | L | — | — | — | M | F | — | N | — | — | — | — | Q | H | 0.255 | 2.711 | 0.275 | 1634 |
| Phage-1437 | L | — | — | — | Q | — | — | K | — | — | — | — | T | — | 0.060 | 0.874 | 0.081 | 1635 |
| Phage-1438 | L | — | — | — | R | F | — | D | — | — | — | — | Q | I | 0.075 | 2.682 | 0.130 | 1636 |
| Phage-1439 | L | — | — | — | R | — | — | R | — | — | — | — | V | S | 0.263 | 2.708 | 0.329 | 1637 |
| Phage-1440 | L | — | — | — | S | — | — | H | — | — | — | — | G | D | 0.078 | 1.102 | 0.090 | 1638 |
| Phage-1441 | L | — | — | — | S | M | — | D | — | — | — | — | N | V | 0.109 | 1.130 | 0.082 | 1639 |
| Phage-1442 | L | — | — | — | S | M | — | R | — | — | — | — | V | G | 0.108 | 0.851 | 0.114 | 1640 |
| Phage-1443 | L | — | — | — | Y | L | — | G | — | — | — | — | Y | R | 0.240 | 2.705 | 0.508 | 1641 |
| Phage-1444 | L | N | — | — | D | T | — | H | — | — | I | — | G | G | 0.072 | 0.620 | 0.076 | 1642 |
| Phage-1445 | L | N | — | — | E | R | — | H | — | — | I | — | G | D | 0.106 | 2.806 | 0.138 | 1643 |
| Phage-1446 | M | — | — | — | F | — | — | G | — | — | I | — | R | Y | 0.205 | 2.723 | 0.359 | 1644 |
| Phage-1447 | M | — | — | — | F | L | — | N | — | — | — | — | D | A | 0.066 | 1.048 | 0.114 | 1645 |
| Phage-1448 | M | — | — | — | G | Y | — | H | — | — | — | — | D | E | 0.081 | 2.709 | 0.265 | 1646 |
| Phage-1449 | M | — | — | — | — | M | — | M | — | — | I | — | A | T | 0.123 | 2.753 | 0.515 | 1647 |
| Phage-1450 | M | — | — | — | L | L | — | D | — | — | — | — | R | M | 0.104 | 2.680 | 0.206 | 1648 |
| Phage-1451 | M | — | — | — | Q | V | — | A | — | — | I | — | N | M | 0.083 | 2.748 | 0.121 | 1649 |
| Phage-1452 | M | — | — | — | R | — | — | N | — | — | — | — | N | E | 0.070 | 2.704 | 0.084 | 1650 |
| Phage-1453 | M | — | — | — | S | F | — | H | — | — | I | — | K | S | 0.172 | 2.728 | 0.283 | 1651 |
| Phage-1454 | P | — | — | — | A | M | — | R | — | — | — | — | V | G | 0.086 | 2.000 | 0.074 | 1652 |
| Phage-1455 | P | — | — | — | H | L | — | R | — | — | — | — | E | W | 0.087 | 2.597 | 0.145 | 1653 |
| Phage-1456 | P | — | — | — | L | L | — | G | — | — | — | — | M | T | 0.073 | 0.621 | 0.080 | 1654 |
| Phage-1457 | P | — | — | — | R | L | — | H | — | — | — | — | L | A | 0.094 | 2.748 | 0.099 | 1655 |
| Phage-1458 | P | — | — | — | Y | — | — | A | — | — | I | — | D | S | 0.081 | 2.741 | 0.099 | 1656 |
| Phage-1459 | P | — | — | — | Y | M | — | M | — | — | I | — | A | — | 0.065 | 2.850 | 0.091 | 1657 |
| Phage-1460 | T | — | — | — | G | — | — | R | — | — | — | — | F | H | 0.096 | 2.628 | 0.086 | 1658 |
| Phage-1461 | — | — | — | — | A | — | F | A | — | — | — | — | M | M | 0.186 | 2.446 | 0.321 | 1659 |
| Phage-1462 | — | — | — | — | A | — | — | H | — | — | — | — | M | G | 0.112 | 2.841 | 0.363 | 1660 |
| Phage-1463 | — | — | — | — | A | — | — | — | — | — | — | — | R | T | 0.075 | 2.721 | 0.078 | 1661 |
| Phage-1464 | — | — | — | — | A | L | — | Q | — | — | — | — | W | — | 0.077 | 2.725 | 0.115 | 1662 |
| Phage-1465 | — | — | — | — | A | M | — | R | — | — | — | — | T | S | 0.144 | 2.702 | 0.232 | 1663 |
| Phage-1466 | — | — | — | — | A | V | — | A | — | — | — | — | A | T | 0.063 | 2.772 | 0.148 | 1664 |
| Phage-1467 | — | — | — | — | A | V | — | E | — | — | — | — | D | T | 0.084 | 2.707 | 0.108 | 1665 |
| Phage-1468 | — | — | — | — | A | V | — | N | — | — | — | — | G | L | 0.098 | 2.675 | 0.108 | 1666 |
| Phage-1469 | — | — | — | — | A | V | — | R | — | — | — | — | — | A | 0.070 | 2.766 | 0.096 | 1667 |
| Phage-1470 | — | — | — | — | E | F | — | H | — | — | I | — | Y | — | 0.153 | 2.663 | 0.160 | 1668 |
| Phage-1471 | — | — | — | — | E | L | — | R | — | — | — | — | Q | V | 0.129 | 2.679 | 0.121 | 1669 |
| Phage-1472 | — | — | — | — | E | L | — | — | — | — | — | — | Y | — | 0.060 | 2.728 | 0.093 | 1670 |
| Phage-1473 | — | — | — | — | E | L | — | W | — | — | — | — | D | R | 0.105 | 1.800 | 0.191 | 1671 |
| Phage-1474 | — | — | — | — | E | M | F | H | — | — | — | — | L | R | 0.067 | 0.996 | 0.157 | 1672 |
| Phage-1475 | — | — | — | — | E | M | — | H | — | — | I | — | Y | — | 0.096 | 2.833 | 0.114 | 1673 |
| Phage-1476 | — | — | — | — | E | M | — | R | — | — | — | — | T | G | 0.086 | 2.624 | 0.081 | 1674 |
| Phage-1477 | — | — | — | — | E | V | F | H | — | — | — | — | D | — | 0.122 | 2.782 | 0.095 | 1675 |
| Phage-1478 | — | — | — | — | E | V | — | H | — | — | — | — | L | G | 0.086 | 2.724 | 0.098 | 1676 |
| Phage-1479 | — | — | — | — | E | V | — | R | — | — | — | — | L | G | 0.074 | 2.705 | 0.080 | 1677 |
| Phage-1480 | — | — | — | — | F | L | — | P | — | — | I | — | Y | — | 0.084 | 2.754 | 0.116 | 1678 |
| Phage-1481 | — | — | — | — | G | F | — | H | — | — | I | — | Y | S | 0.097 | 2.706 | 0.377 | 1679 |
| Phage-1482 | — | — | — | — | G | — | — | Q | — | — | — | — | I | — | 0.071 | 2.689 | 0.124 | 1680 |
| Phage-1483 | — | — | — | — | G | — | — | R | — | — | I | — | E | N | 0.071 | 2.749 | 0.156 | 1681 |
| Phage-1484 | — | — | — | — | G | L | F | R | — | — | I | — | T | D | 0.104 | 2.722 | 0.180 | 1682 |
| Phage-1485 | — | — | — | — | G | L | — | H | — | — | — | — | W | T | 0.102 | 2.703 | 0.179 | 1683 |
| Phage-1486 | — | — | — | — | — | — | F | Q | — | — | — | — | V | D | 0.083 | 0.508 | 0.105 | 1684 |
| Phage-1487 | — | — | — | — | — | — | — | A | — | — | I | — | E | S | 0.076 | 2.824 | 0.123 | 1685 |
| Phage-1488 | — | — | — | — | — | — | — | — | — | — | — | — | S | Y | 0.072 | 2.684 | 0.102 | 1686 |
| Phage-1489 | — | — | — | — | — | L | F | Q | — | — | — | — | A | G | 0.131 | 2.703 | 0.192 | 1687 |
| Phage-1490 | — | — | — | — | — | L | — | K | — | — | — | — | A | V | 0.074 | 2.707 | 0.156 | 1688 |
| Phage-1491 | — | — | — | — | — | L | — | K | — | — | — | — | T | A | 0.067 | 2.685 | 0.154 | 1689 |
| Phage-1492 | — | — | — | — | — | L | — | N | — | — | — | — | P | G | 0.082 | 2.828 | 0.218 | 1690 |
| Phage-1493 | — | — | — | — | — | L | — | R | — | — | — | — | G | L | 0.086 | 2.728 | 0.093 | 1691 |
| Phage-1494 | — | — | — | — | — | V | — | R | — | — | — | — | L | A | 0.114 | 0.865 | 0.096 | 1692 |
| Phage-1495 | — | — | — | — | L | — | — | H | — | — | — | — | Y | V | 0.087 | 2.707 | 0.180 | 1693 |
| Phage-1496 | — | — | — | — | L | — | — | N | — | — | — | — | L | — | 0.094 | 1.112 | 0.115 | 1694 |
| Phage-1497 | — | — | — | — | L | L | — | D | — | — | — | — | N | G | 0.065 | 2.819 | 0.208 | 1695 |
| Phage-1498 | — | — | — | — | L | L | — | D | — | — | — | — | S | K | 0.076 | 2.844 | 0.194 | 1696 |
| Phage-1499 | — | — | — | — | L | L | — | R | — | — | — | — | V | — | 0.071 | 1.580 | 0.102 | 1697 |
| Phage-1500 | — | — | — | — | L | M | — | H | — | — | — | — | S | — | 0.075 | 2.740 | 0.131 | 1698 |
| Phage-1501 | — | — | — | — | L | V | — | R | — | — | I | — | T | — | 0.093 | 2.840 | 0.148 | 1699 |
| Phage-1502 | — | — | — | — | M | L | — | A | — | — | — | — | — | — | 0.062 | 2.682 | 0.114 | 1700 |
| Phage-1503 | — | — | — | — | M | L | — | H | — | — | I | — | Y | — | 0.120 | 2.745 | 0.104 | 1701 |
| Phage-1504 | — | — | — | — | M | V | — | R | — | — | — | — | D | N | 0.075 | 2.700 | 0.074 | 1702 |
| Phage-1505 | — | — | — | — | Q | L | — | D | — | — | — | — | N | S | 0.070 | 2.672 | 0.075 | 1703 |
| Phage-1506 | — | — | — | — | Q | L | — | N | — | — | — | — | L | K | 0.079 | 0.938 | 0.090 | 1704 |
| Phage-1507 | — | — | — | — | Q | M | — | R | — | — | — | — | Y | G | 0.129 | 2.725 | 0.117 | 1705 |
| Phage-1508 | — | — | — | — | Q | M | — | — | — | — | — | — | A | T | 0.061 | 2.711 | 0.148 | 1706 |
| Phage-1509 | — | — | — | — | R | — | — | H | — | — | — | — | K | H | 0.188 | 2.298 | 0.291 | 1707 |
| Phage-1510 | — | — | — | — | R | L | — | D | — | — | — | — | M | T | 0.080 | 2.827 | 0.147 | 1708 |
| Phage-1511 | — | — | — | — | R | L | — | D | — | — | — | — | S | G | 0.078 | 2.730 | 0.103 | 1709 |
| Phage-1512 | — | — | — | — | R | L | — | M | — | — | I | — | L | E | 0.083 | 2.734 | 0.123 | 1710 |
| Phage-1513 | — | — | — | — | R | L | — | N | — | — | — | — | I | T | 0.094 | 2.703 | 0.128 | 1711 |
| Phage-1514 | — | — | — | — | R | L | — | R | — | — | I | — | Q | H | 0.165 | 2.714 | 0.284 | 1712 |
| Phage-1515 | — | — | — | — | R | L | — | — | — | — | — | — | D | P | 0.064 | 1.575 | 0.084 | 1713 |
| Phage-1516 | — | — | — | — | R | M | — | A | — | — | I | — | D | P | 0.105 | 2.738 | 0.161 | 1714 |
| Phage-1517 | — | — | — | — | R | M | — | A | — | — | — | — | S | H | 0.084 | 2.713 | 0.119 | 1715 |
| Phage-1518 | — | — | — | — | R | M | — | D | — | — | — | — | T | S | 0.073 | 1.039 | 0.080 | 1716 |
| Phage-1519 | — | — | — | — | R | M | — | — | — | — | — | — | T | T | 0.111 | 1.917 | 0.133 | 1717 |
| Phage-1520 | — | — | — | — | S | — | — | R | — | — | — | — | N | G | 0.076 | 2.686 | 0.069 | 1718 |
| Phage-1521 | — | — | — | — | S | L | — | H | — | — | — | — | M | R | 0.090 | 2.592 | 0.146 | 1719 |
| Phage-1522 | — | — | — | — | S | L | — | — | — | — | — | — | E | A | 0.066 | 2.711 | 0.210 | 1720 |
| Phage-1523 | — | — | — | — | S | M | — | A | — | — | — | — | N | Y | 0.080 | 2.708 | 0.163 | 1721 |
| Phage-1524 | — | — | — | — | S | M | — | D | — | — | — | — | T | A | 0.067 | 2.649 | 0.079 | 1722 |
| Phage-1525 | — | — | — | — | S | M | — | R | — | — | — | — | Y | E | 0.080 | 2.733 | 0.079 | 1723 |
| Phage-1526 | — | — | — | — | S | V | — | G | — | — | — | — | N | L | 0.080 | 0.754 | 0.094 | 1724 |
| Phage-1527 | — | — | — | — | S | V | — | Y | — | — | I | — | Q | N | 0.068 | 2.730 | 0.116 | 1725 |
| Phage-1528 | — | — | — | — | T | — | F | H | — | — | — | — | Q | S | 0.150 | 2.730 | 0.195 | 1726 |
| Phage-1529 | — | — | — | — | T | — | — | Q | — | — | I | — | R | S | 0.098 | 2.830 | 0.175 | 1727 |
| Phage-1530 | — | — | — | — | T | L | — | Q | — | — | — | — | L | K | 0.083 | 2.619 | 0.093 | 1728 |
| Phage-1531 | — | — | — | — | W | L | — | Q | — | — | I | — | N | G | 0.154 | 2.823 | 0.196 | 1729 |
| Phage-1532 | — | — | — | — | W | L | — | Q | — | — | — | — | N | S | 0.151 | 2.725 | 0.139 | 1730 |
| Phage-1533 | — | — | — | — | W | L | — | R | — | — | — | — | T | — | 0.624 | 2.695 | 0.795 | 1731 |
| Phage-1534 | — | — | — | — | W | M | — | D | — | — | — | — | R | V | 0.166 | 2.067 | 0.210 | 1732 |
| Phage-1535 | — | — | — | — | Y | L | — | G | — | — | — | — | M | L | 0.139 | 2.476 | 0.131 | 1733 |
| Phage-1536 | — | — | — | — | Y | L | — | Q | — | — | — | — | G | K | 0.061 | 2.559 | 0.243 | 1734 |
| Phage-1537 | — | — | — | — | Y | L | — | V | — | — | — | — | L | R | 0.107 | 2.667 | 0.157 | 1735 |
| Phage-1538 | — | — | — | — | Y | M | — | G | — | — | — | — | N | P | 0.078 | 2.780 | 0.162 | 1736 |
| Phage-1539 | — | — | — | — | Y | M | — | G | — | — | — | — | R | L | 0.075 | 2.735 | 0.070 | 1737 |
| Phage-1540 | — | — | W | — | A | L | — | D | — | — | — | — | E | — | 0.094 | 2.714 | 0.116 | 1738 |
| Phage-1541 | — | — | W | — | H | — | — | R | — | — | — | — | D | M | 0.131 | 2.001 | 0.222 | 1739 |
| Phage-1542 | — | — | W | — | T | L | — | A | — | — | — | — | Q | — | 0.146 | 2.466 | 0.195 | 1740 |
| Phage-1543 | — | N | — | — | Q | — | — | E | — | — | I | — | T | S | 0.187 | 1.474 | 0.138 | 1741 |
| Phage-1544 | W | K | — | — | A | T | S | M | — | — | N | — | V | D | 0.133 | 2.852 | 0.223 | 1742 |
| Phage-1545 | I | — | — | — | I | L | — | D | — | — | — | — | E | G | 0.146 | 2.661 | 0.121 | 1743 |
| Phage-1546 | A | — | L | — | Y | L | Q | P | Q | — | — | — | E | T | 0.065 | 0.058 | 0.084 | 1744 |
| Phage-1547 | E | — | — | — | M | R | — | N | — | — | — | — | R | E | 0.074 | 0.098 | 0.088 | 1745 |
| Phage-1548 | E | — | — | — | Q | T | — | R | — | — | I | — | I | Y | 0.072 | 0.094 | 0.100 | 1746 |
| Phage-1549 | G | T | — | — | P | T | S | Q | — | R | Y | — | I | I | 0.108 | 0.106 | 0.421 | 1747 |
| Phage-1550 | I | — | — | — | E | R | — | — | — | — | — | — | V | T | 0.077 | 0.184 | 0.093 | 1748 |
| Phage-1551 | L | — | — | — | W | L | — | R | — | — | — | — | E | N | 0.464 | 1.967 | 0.712 | 1749 |
| Phage-1552 | L | E | — | — | A | W | Q | L | — | S | — | — | D | D | 0.081 | 0.075 | 0.136 | 1750 |
| Phage-1553 | L | N | — | — | A | F | — | D | — | — | I | — | — | T | 0.872 | 2.693 | 1.387 | 1751 |
| Phage-1554 | P | — | — | — | E | M | — | N | — | — | — | — | D | R | 0.071 | 0.493 | 0.072 | 1752 |
| Phage-1555 | P | — | — | — | L | L | — | K | — | — | I | — | P | S | 0.074 | 0.381 | 0.082 | 1753 |
| Phage-1556 | — | — | — | — | A | L | — | H | — | — | I | — | D | T | 1.367 | 2.725 | 1.630 | 1754 |
| Phage-1557 | — | — | — | — | G | L | — | K | — | — | — | — | S | M | 0.102 | 0.480 | 0.108 | 1755 |
| Phage-1558 | — | — | — | — | G | M | — | K | — | — | I | — | N | K | 0.075 | 0.249 | 0.082 | 1756 |
| Phage-1559 | — | — | — | — | S | V | — | D | — | — | — | — | W | G | 0.074 | 0.332 | 0.077 | 1757 |
| Phage-1560 | — | — | — | — | W | — | — | G | — | — | — | — | Q | S | 0.124 | 0.298 | 0.191 | 1758 |
| Phage-1561 | — | — | — | — | W | L | — | R | — | — | — | — | F | D | 1.299 | 2.660 | 1.660 | 1759 |
| Phage-1562 | — | — | W | — | G | L | — | N | — | — | — | — | T | W | 0.468 | 2.082 | 0.674 | 1760 |
| Phage-1563 | — | E | Q | — | G | L | Q | — | L | R | G | — | K | V | 0.070 | 0.071 | 0.097 | 1761 |
| Phage-1564 | W | — | — | — | F | L | — | — | — | — | — | — | D | S | 0.329 | 0.858 | 0.465 | 1762 |
| Phage-1565 | I | — | — | — | Q | — | — | G | — | — | — | — | L | G | 0.080 | 2.478 | 0.078 | 1763 |
| Phage-1566 | I | — | — | — | L | L | — | K | — | — | — | — | D | L | 0.070 | 2.731 | 0.332 | 1764 |
| Phage-1567 | — | — | — | — | E | L | — | H | — | — | I | — | Y | — | 0.114 | 2.730 | 0.332 | 1765 |
| Phage-1568 | — | — | — | — | T | L | — | D | — | — | — | — | R | K | 0.080 | 2.642 | 0.088 | 1766 |
| Phage-1569 | L | — | — | — | E | V | N | R | — | — | I | — | G | V | 0.080 | 2.698 | 0.075 | 1767 |
| Phage-1570 | I | — | — | — | M | M | F | D | — | — | — | — | N | K | 0.122 | 2.062 | 0.152 | 1768 |
| Phage-1571 | L | — | — | — | F | F | — | D | — | — | — | — | R | N | 0.072 | 2.841 | 0.221 | 1769 |
| Phage-1572 | P | — | — | — | V | L | — | H | — | — | I | — | A | A | 0.075 | 2.715 | 0.070 | 1770 |
| Phage-1573 | — | — | — | — | A | — | — | — | — | — | I | — | K | T | 0.134 | 2.694 | 0.169 | 1771 |
| Phage-1574 | — | — | — | — | L | F | — | R | — | — | I | — | T | E | 0.125 | 2.717 | 0.154 | 1772 |
| Phage-1575 | — | — | — | — | L | — | — | G | — | — | I | — | S | S | 0.075 | 2.718 | 0.124 | 1773 |
| Phage-1576 | W | I | — | V | G | L | M | L | A | L | — | G | T | I | 0.067 | 2.717 | 0.092 | 1774 |

(—) indicates same amino acid as in TROP2 Fab Peptide-2 corresponding position (e.g. Phage-981 position).

TABLE 30
Sequences of those peptides selected for synthesis
(TROP2 Fab Peptide-2 Optimization)

| Construct | | |
|---|---|---|
| Description | Amino Acid Sequence | SEQ ID NO: |
| Peptide-50 | IDFCAMYQWPICDT | 179 |
| Peptide-51 | IDFCAVYKWPVCQV | 180 |
| Peptide-52 | IDFCMLYNWPICAG | 181 |
| Peptide-53 | VDFCKIYAWPICGS | 182 |
| Peptide-54 | VDFCKLYNWPVCQT | 183 |
| Peptide-55 | IDFCLIYNWPVCDT | 184 |
| Peptide-56 | EDFCKLYNWPICYQ | 185 |
| Peptide-57 | VDFCGLYHWPICYQ | 186 |
| Peptide-58 | VDFCYLYNWPVCSK | 187 |
| Peptide-59 | IDFCAIYQWPVCRS | 188 |
| Peptide-60 | VDFCALYNWPVCET | 189 |
| Peptide-61 | PDFCAVYRWPICYQ | 190 |
| Peptide-62 | VDFCELYRWPICNS | 191 |
| Peptide-63 | LDFCKIYDWPICHL | 192 |
| Peptide-64 | LDFCKLYQWPVCFT | 193 |
| Peptide-65 | IDFCLLYDWPVCAS | 194 |
| Peptide-66 | IDFCLLYDWPICGR | 195 |
| Peptide-67 | MDFCQIYDWPICRL | 196 |
| Peptide-68 | PDFCQLYNWPVCAG | 197 |
| Peptide-69 | VDFCSFYRWPICET | 198 |
| Peptide-70 | IDFCSLYQWPVCGT | 199 |
| Peptide-71 | VDFCTIYKWPVCEG | 200 |
| Peptide-72 | VDFCYQYGWPICSR | 201 |

### Example 10: TROP2 Polypeptide Complex Binding (PC25. PC26, PC27, PC28, PC29, and PC30)

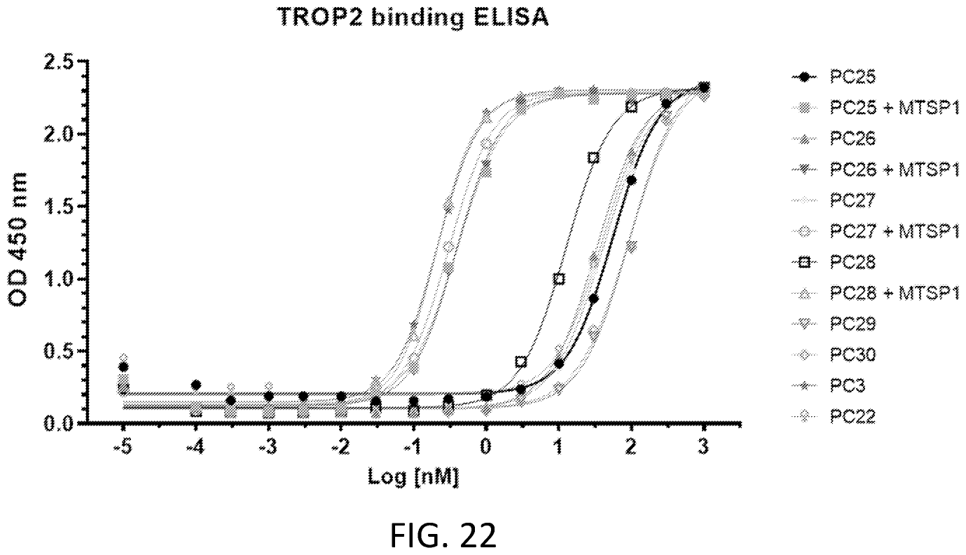

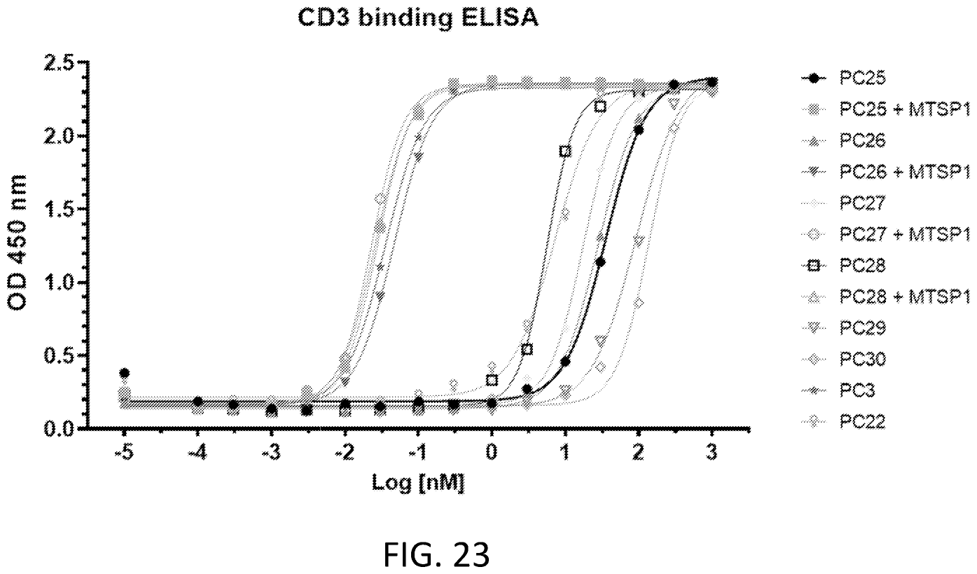

PC25, PC26, PC27, PC28, PC29, and PC30, the sequences of which are provided in Table 6 were evaluated for TROP2 and CD3ε binding according to the methods of Example 1. FIGS. 22 and 23 show representative ELISAs of TROP2 and CD3ε binding, respectively.

### Example 11: Polypeptide Complex Mediated Tumor Cytotoxicity

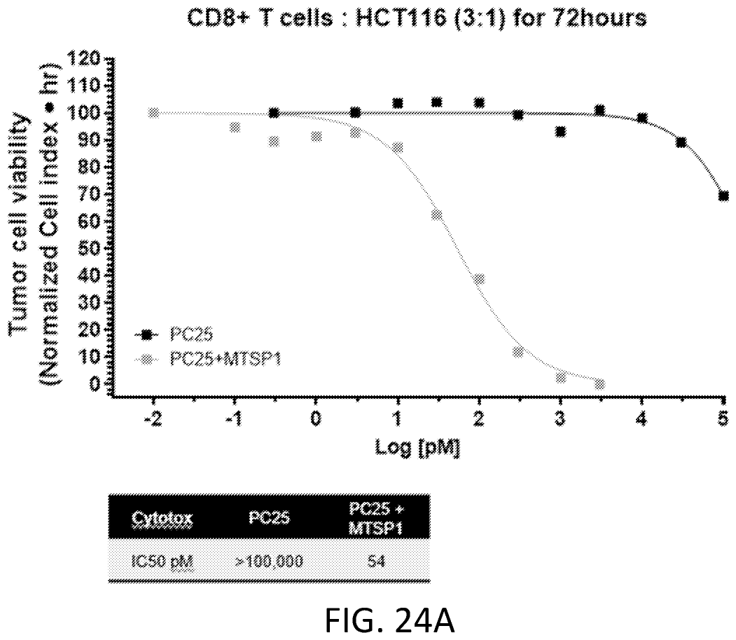

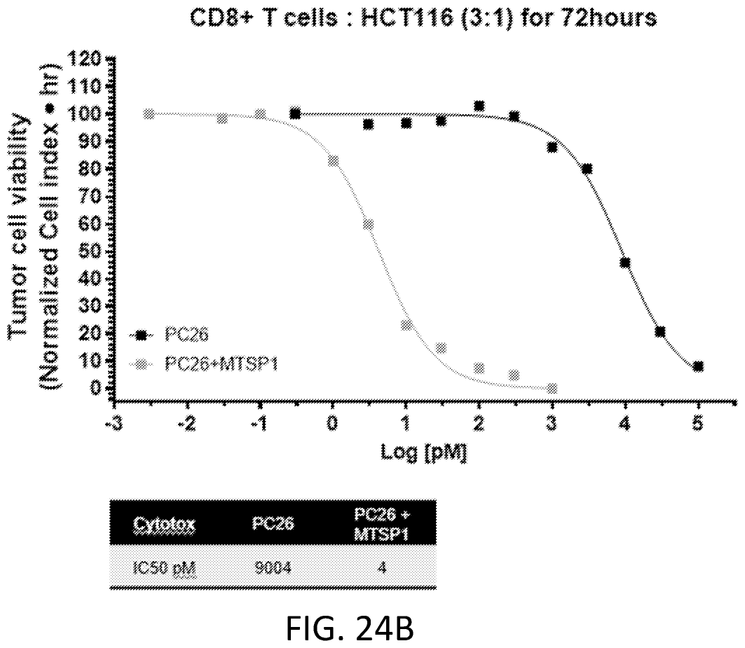

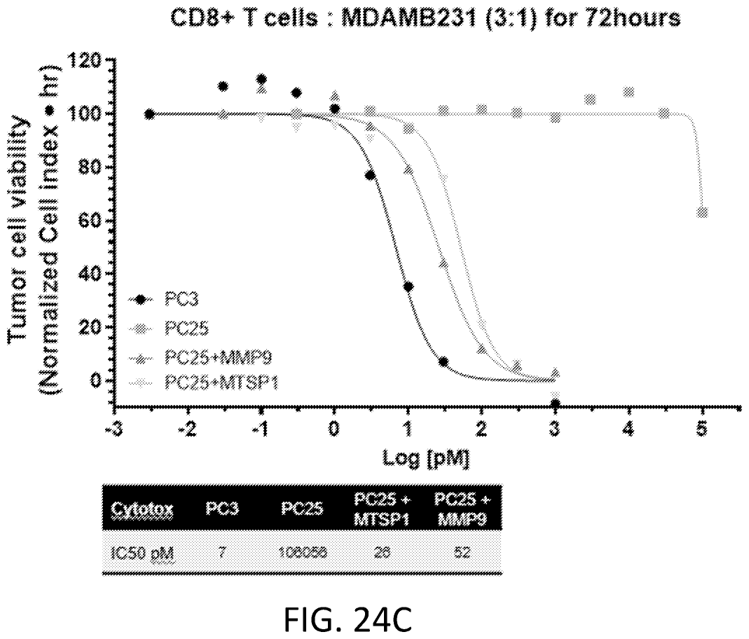

Polypeptide complexes were evaluated in a functional in vitro tumor cell killing assay using the TROP2 positive tumor cell lines HCT116, NCI-1H292, and MDAMB231. Tumor cell killing was measured using an xCelligence real time cell analyzer from Agilent that relies on sensor impedance measurements (cell index) that increased as tumor cells adhere, spread, and expand on the surface of the sensor. Likewise, as the tumor cells were killed the impedance decreased. 10,000 tumor cells were added per well and allowed to adhere overnight on a 96 well E-Plate. The following day polypeptide complexes titrated in human serum supplemented medium along with 30,000 CD8+ T cells were added to the wells. Cell index measurements were taken every 10 minutes for an additional 72 hours. The cell index times number of hours (tumor cell growth kinetics) was then plotted versus concentration of polypeptide complex where the concentration required to reduce the tumor growth 50% (IC50) was calculated using Graphpad Prism software. FIG. 24A illustrates PC25 mediated HCT116 tumor cell killing in the presence of CD8+ T cells. FIG. 24B illustrates PC26 mediated HCT116 tumor cell killing in the presence of CD8+ T cells. FIG. 24C illustrates PC25 mediated MDAMB231 tumor cell killing in the presence of CD8+ T cells.

### Example 12: Polypeptide Complex-22 (PC22) Pharmacokinetics in Cynomolgus Monkey

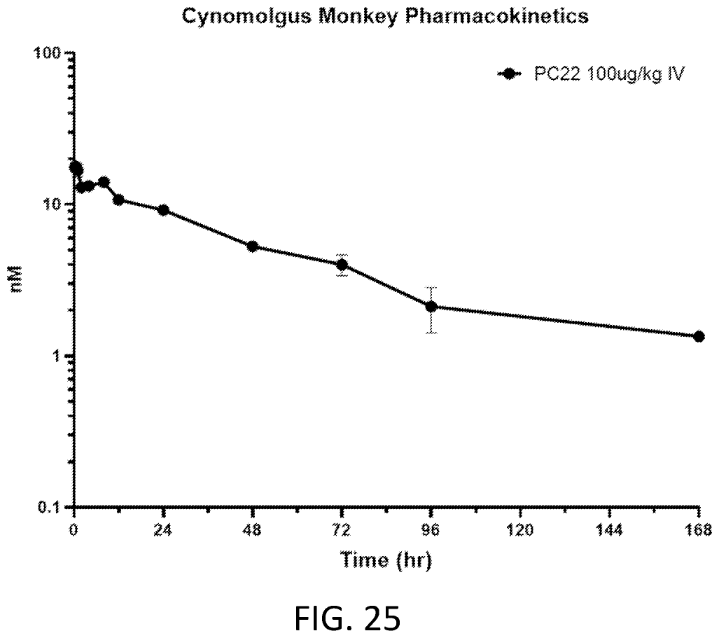

Pharmacokinetics and exploratory safety of polypeptide molecules were evaluated in cynomolgus monkeys. Briefly, cynomolgus monkeys of approximately 3 kg bodyweight were administered polypeptides as an IV bolus and observed daily for signs of adverse events. No in-life adverse events were observed. After dosing, blood was collected in K2 EDTA tubes at specific timepoints and processed to plasma. Plasma was stored frozen until analysis. Concentration of polypeptide molecules in plasma was measured via standard ELISA techniques relative to a reference standard diluted in control cyno plasma. Plasma concentration curves were fit to a standard two phase exponential equation representing distribution and elimination phases. Fitting of pharmacokinetics enabled the calculation of Cmax, half-life, volume of distribution, clearance, and 7 day area under the curve (AUC) shown in Table 31 and in FIG. 25. Measured pharmacokinetics in cyno support once weekly dosing in humans.

TABLE 31
PC22 PK calculations

| | PC22 100 ug/kg | Units |
|---|---|---|
| C_{MAX} | 17.38 | nM |
| t_{1/2} | 68.97 | hr |
| Vd | 0.18 | L |
| VSS | 0.44 | L |
| CL | 0.61 | mL/hr/kg |
| BW | 3.00 | kg |
| 7 day | 45640 | nM · min |
| AUC | | |

### Example 13: PC22 in Cynomolgus Cytokine Release

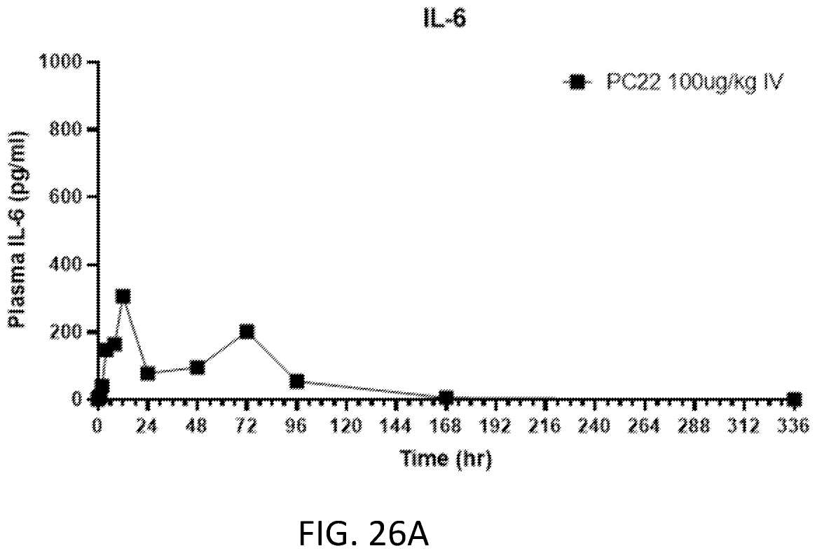

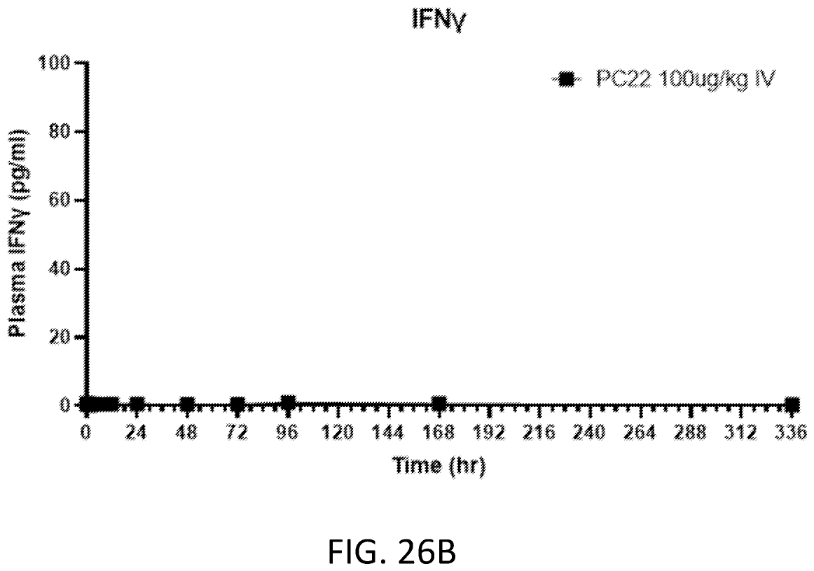

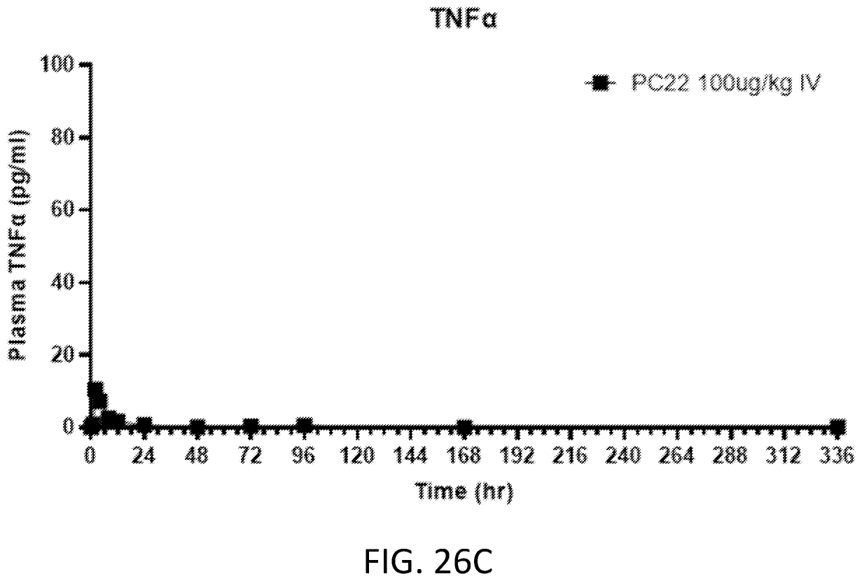

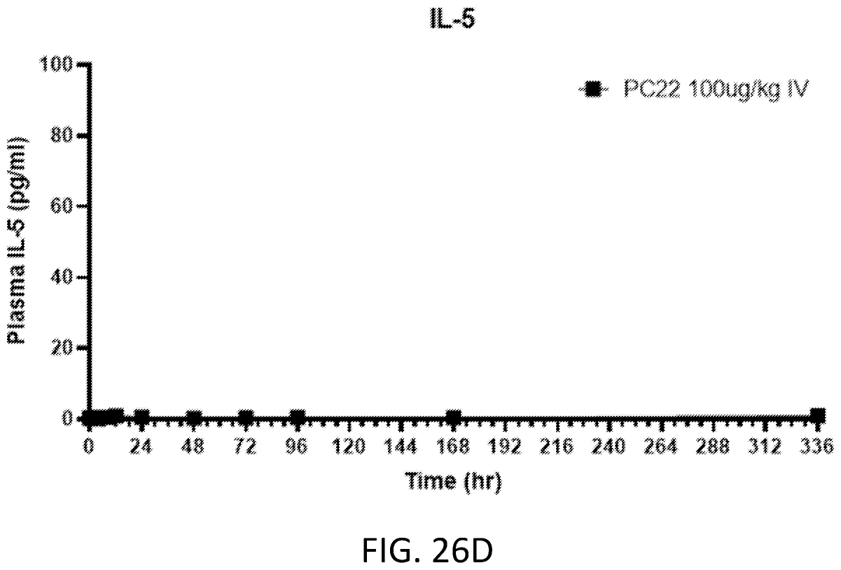

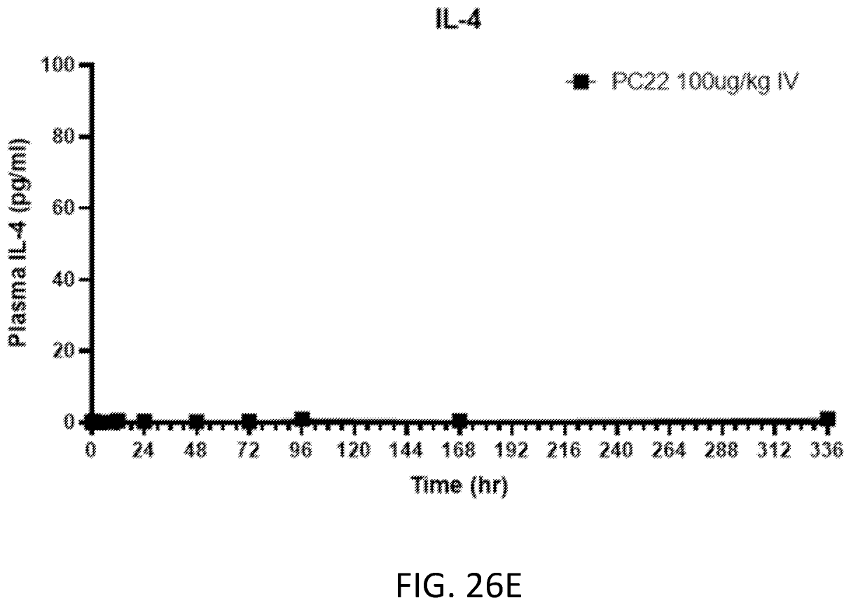

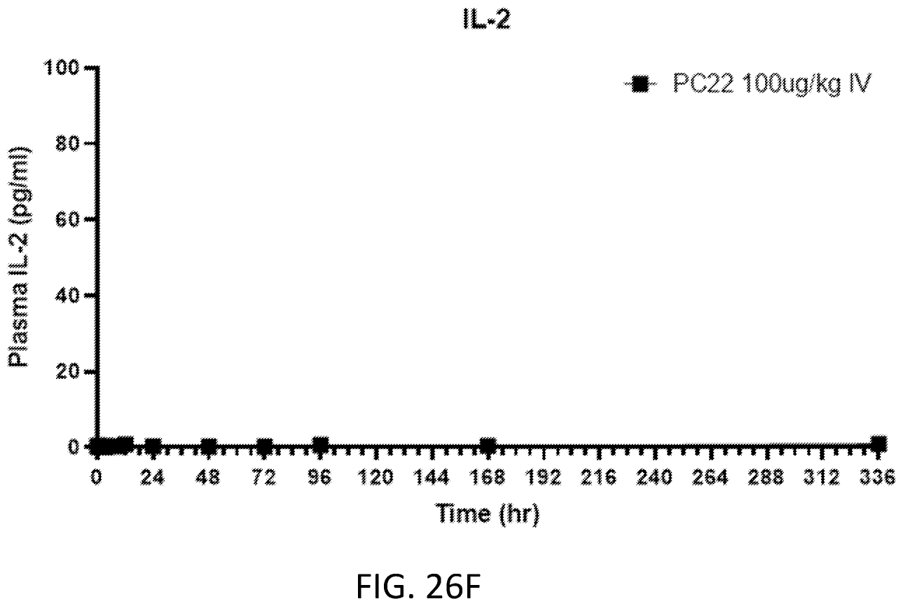

Cytokine release after polypeptide molecule administration by IV bolus was evaluated in cynomolgus monkeys. Briefly, cynomolgus monkeys of approximately 3 kg bodyweight were administered polypeptides as an IV bolus and observed daily for signs of adverse events. No in-life adverse events were observed. After dosing, blood was collected in K2 EDTA tubes at specific timepoints and processed to plasma. Plasma was stored frozen until analysis. Plasma samples were analyzed for cytokines using a non-human primate cytometric Th1/Th2 bead array kit from BD biosciences following the manufacturer's instructions. Interferon gamma, tumor necrosis factor alpha, interleukin 6, interleukin 5, interleukin 4, and interleukin 2 levels in plasma were calculated relative to reference standards provided with the bead array kit. FIGS. 26A-26F illustrate cytokine release in cynomolgus monkeys after single IV bolus of PC22.

### Example 14: PC22 in Cynomolgus Toxicity

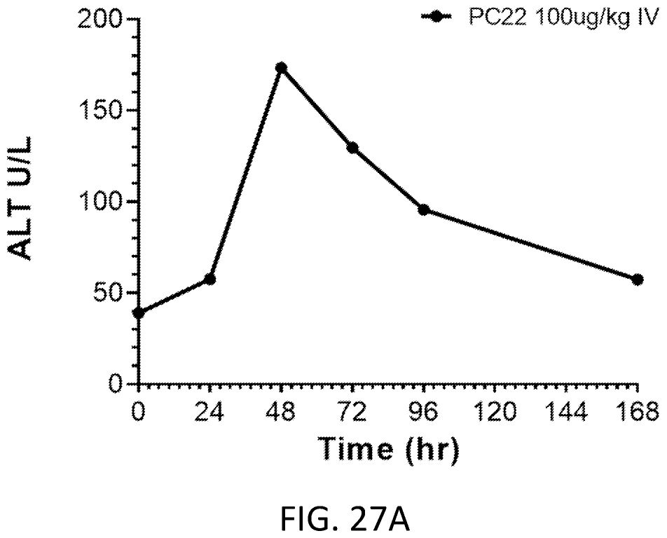

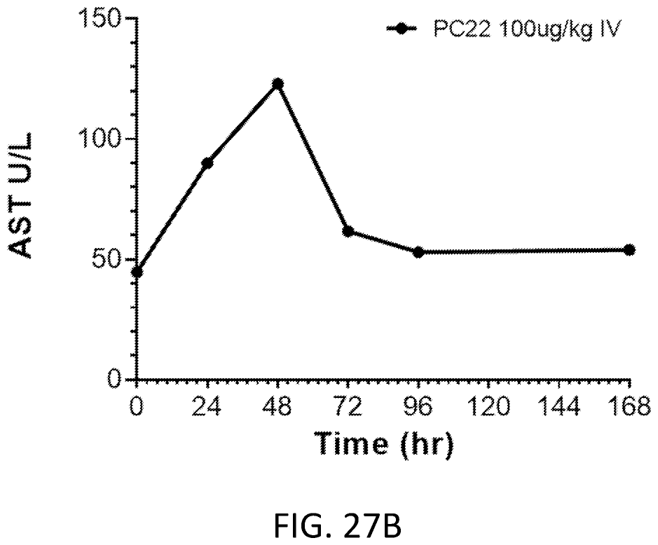

Systemic liver enzymes after PC22 administration by IV bolus was evaluated in cynomolgus monkeys. Briefly, cynomolgus monkeys of approximately 3 kg bodyweight were administered polypeptides as an IV bolus and observed daily for signs of adverse events. No in-life adverse events were observed. After dosing, blood was collected in K2 EDTA tubes at specific timepoints and processed to plasma. Plasma was stored frozen until analysis. Plasma samples were analyzed for the presence of liver enzymes aspartate transaminase (AST) and alanine aminotransferase (ALT) as signs of potential liver toxicity. AST and ALT levels were remained within the normal ranges for all timepoints tested after dosing suggesting a lack of liver toxicity. AST and ALT were quantified following the instructions provided in a commercially available kit from Millipore. AST and ALT levels were calculated according to manufacturer's instructions relative to a positive control reference standard. FIGS. 27A-27B illustrate serum liver enzyme levels in cynomolgus monkeys after single IV bolus of PC22.

### Example 15: Anti-Tumor Efficacy in a Mouse Model of Human Triple Negative Breast Cancer

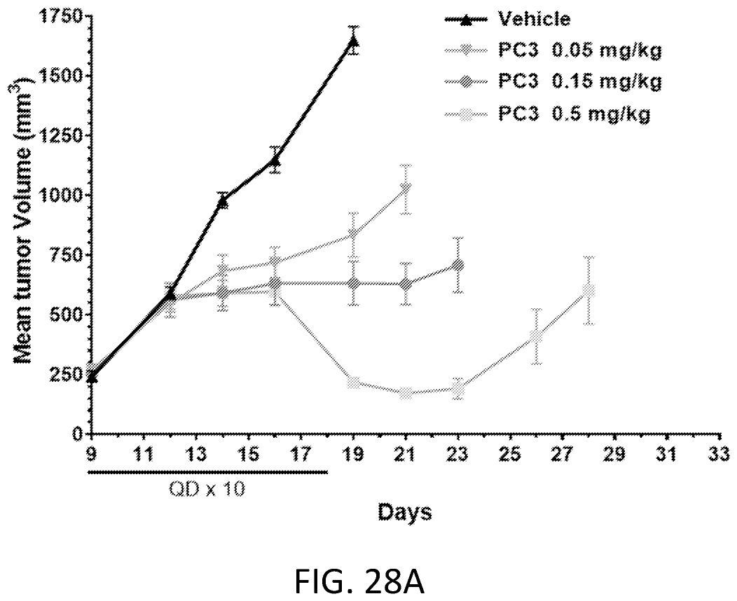

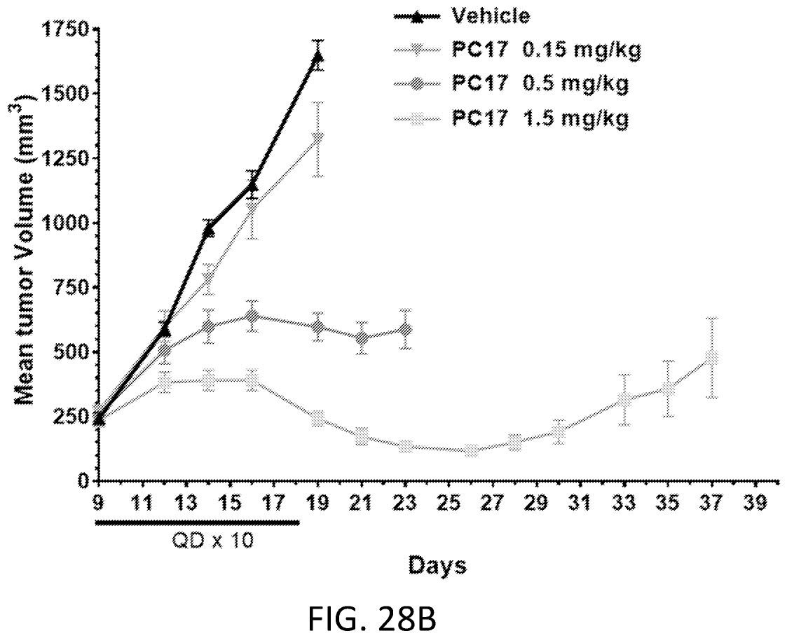

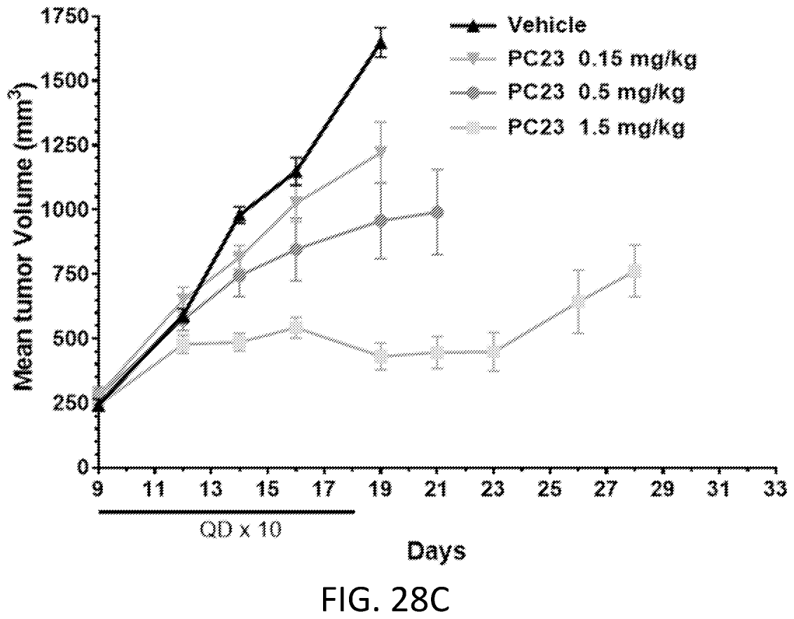

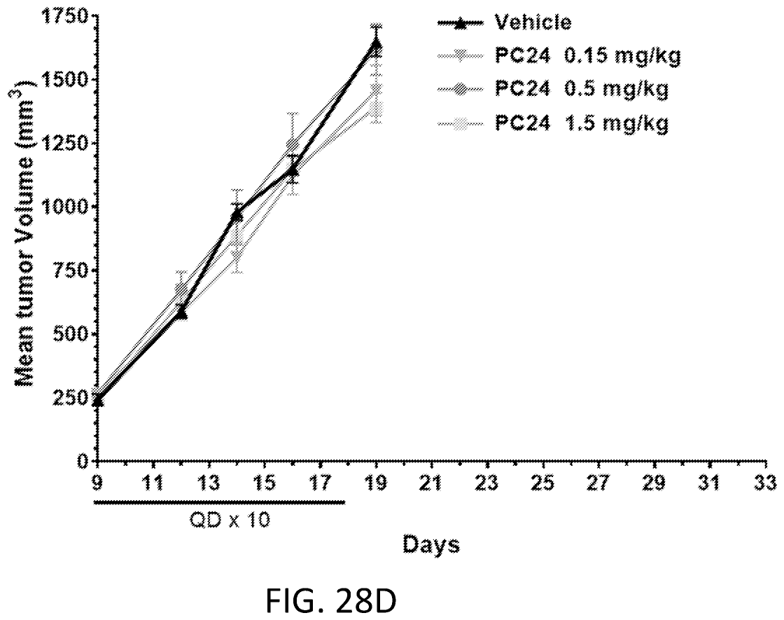

Female NCG mice were subcutaneously implanted with 5 million MDA-MB-231 triple negative tumor cells. When tumors reached 50-80 mm³, 20 million human PBMCs were engrafted via the tail vein. When tumors reached 275 mm3, mice were randomized into groups and compounds were dosed intravenously every day for 10 days. Tumor volume was measured every two to three days and plotted overtime. The tumor volume growth kinetics indicate anti-tumor activity of masked TROP2 targeted bispecific compounds. The anti-tumor activity observed was protease dependent in that the compound lacking the protease substrate within the cleavable linker was equivalent to vehicle controls. Shown are PC3 (FIG. 28A), PC17 (FIG. 28B), PC23 (FIG. 28C), PC24 (FIG. 28D).

### Example 16: Optimized Phage Library Construction—CD3 scFv Peptides

Sequence activity relationships (SAR) were established for Peptide-A and Peptide-B by mutating each individual residue within the peptide to alanine and measuring binding and inhibition against SP34.185 scFv. Peptide residues whose alanine mutations significantly weakened binding and inhibition can be considered critical residues where mutations were not tolerated. Peptide residues whose alanine mutations performed similarly to the non-mutated sequence can be considered non-critical sites where mutations were indeed tolerated. Using the peptide SAR, DNA oligo libraries were constructed where codons encoding critical residues within each peptide sequence were minimally mutated and codons encoding non-critical residues were heavily mutated. The resulting oligos were cloned into bacteriophage vectors used to display the SAR guided peptides via fusion to the pIII filament of the bacteriophage. The relevant vectors were then used to produce the phage optimization libraries via amplification in bacteria using standard techniques in the field.

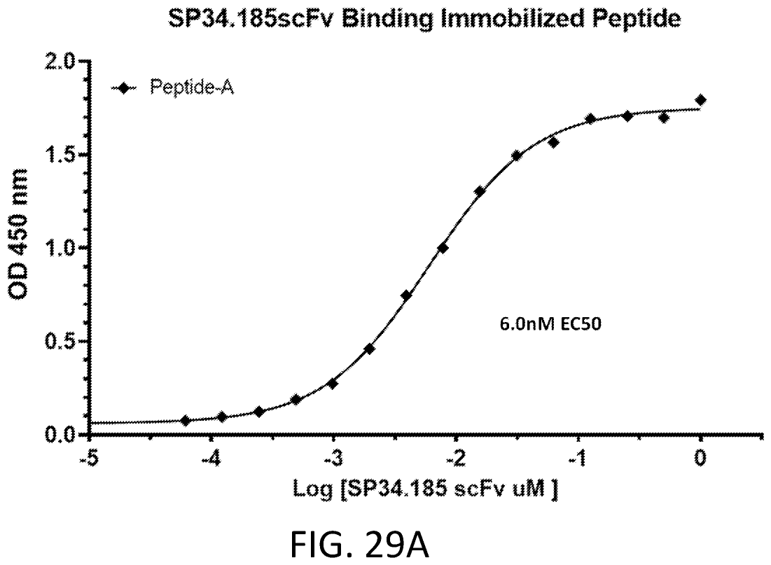

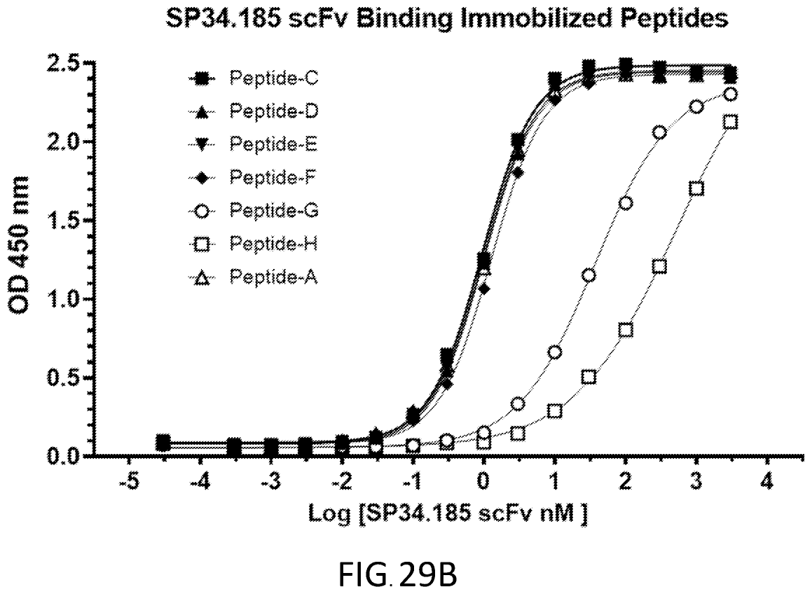

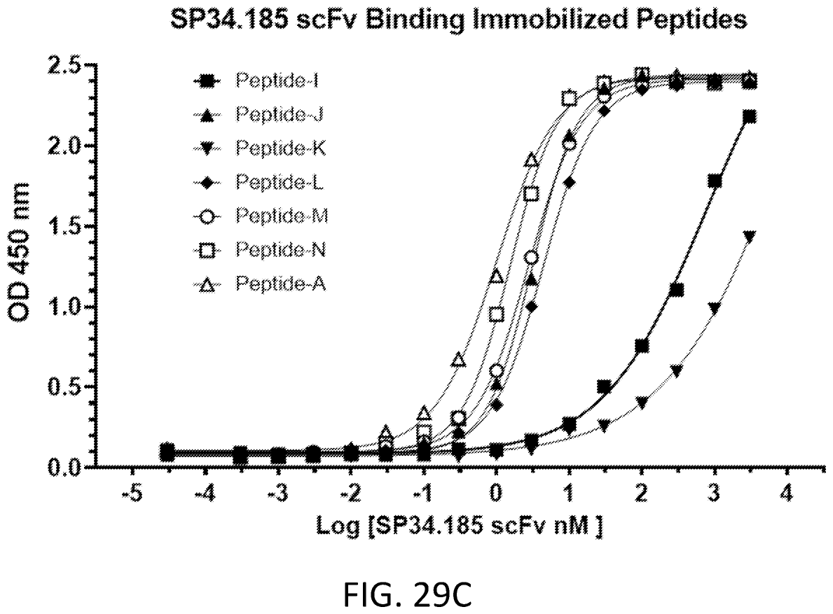

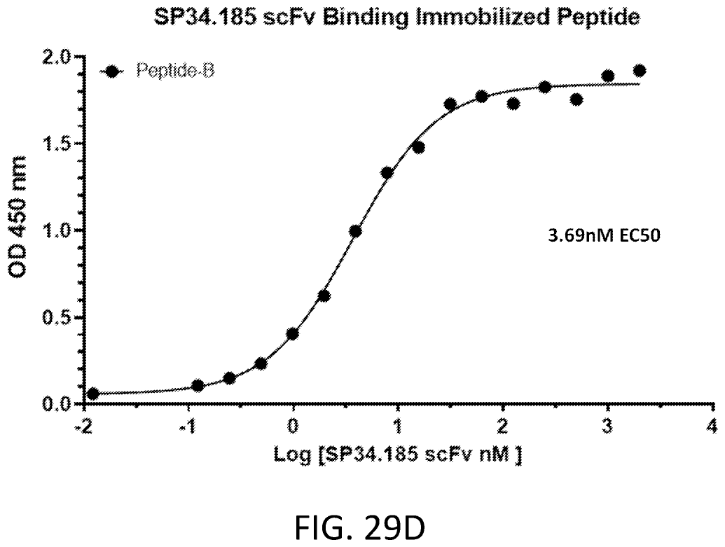

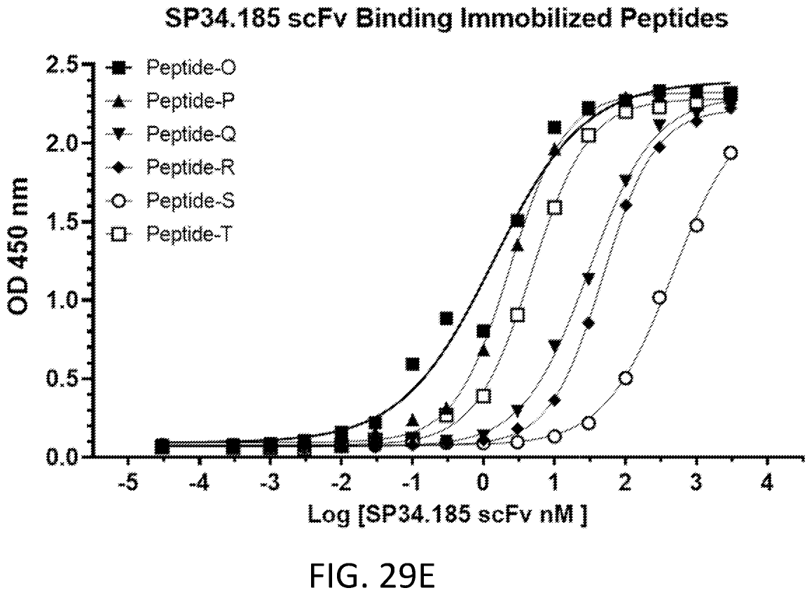

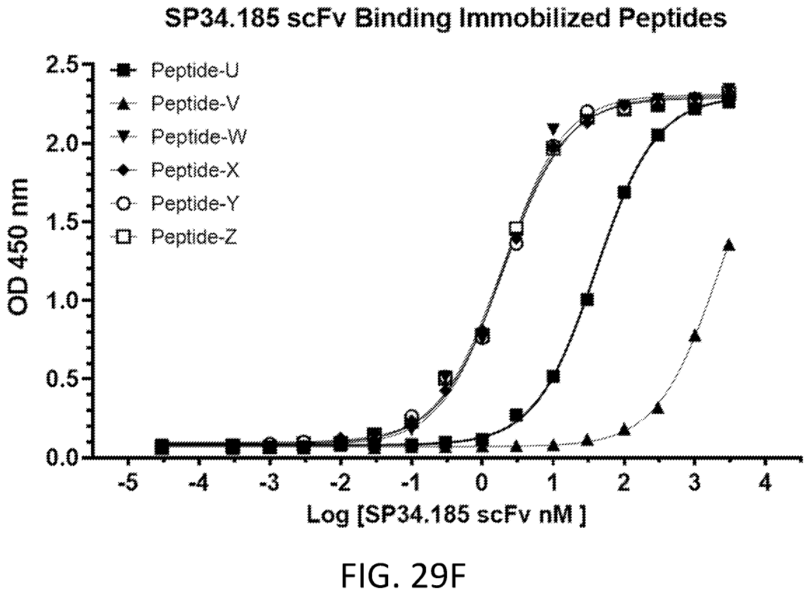

Peptides were evaluated for their ability to bind SP34.185 scFv by standard enzyme linked immunosorbent assays (ELISAs). Briefly, biotinylated peptides were captured on neutravidin coated plates, quench with biocytin followed by a washing step. SP34.185 scFv was then titrated onto the peptide captured plates. Plates were then washed and bound SP34.185 scFv was detected using a secondary horse radish peroxidase antibody conjugate. After washing again, plates were developed using standard ELISA techniques and stopped using acid. The concentration of SP34.185 scFv required to achieve 50% maximal signal or EC50 was calculated using Graphpad prism. Data is shown in FIGS. 29A-29F and summarized in Tables 32A-32D. Peptide Sequences of CD3 Ala Scan Peptides for Peptide A and Peptide-B are shown in Table 34.

TABLE 32A
Summary of FIG. 29B

| ELISA | Peptide-A | Peptide-C | Peptide-D | Peptide-E | Peptide-F | Peptide-G | Peptide-H |
|---|---|---|---|---|---|---|---|
| EC50 nM | 1.013 | 0.9429 | 1.018 | 0.9738 | 1.27 | 47.5 | 346.2 |

TABLE 32B
Summary of FIG. 29C

| ELISA | Peptide-A | Peptide-I | Peptide-J | Peptide-K | Peptide-L | Peptide-M | Peptide-N |
|---|---|---|---|---|---|---|---|
| EC50 nM | 0.986 | 310.8 | 3.134 | 1.960 | 4.363 | 2.76 | 1.546 |

TABLE 32C
Summary of FIG. 29E

| ELISA | Peptide-O | Peptide-P | Peptide-Q | Peptide-R | Peptide-S | Peptide-T |
|---|---|---|---|---|---|---|
| EC50 nM | 1.356 | 2.359 | 30.04 | 47.50 | 457.1 | 4.762 |

TABLE 32D
Summary of FIG. 29F

| ELISA | Peptide-U | Peptide-V | Peptide-W | Peptide-X | Peptide-Y | Peptide-Z |
|---|---|---|---|---|---|---|
| EC50 nM | 39.90 | 2168 | 1.916 | 1.948 | 2.012 | 1.833 |

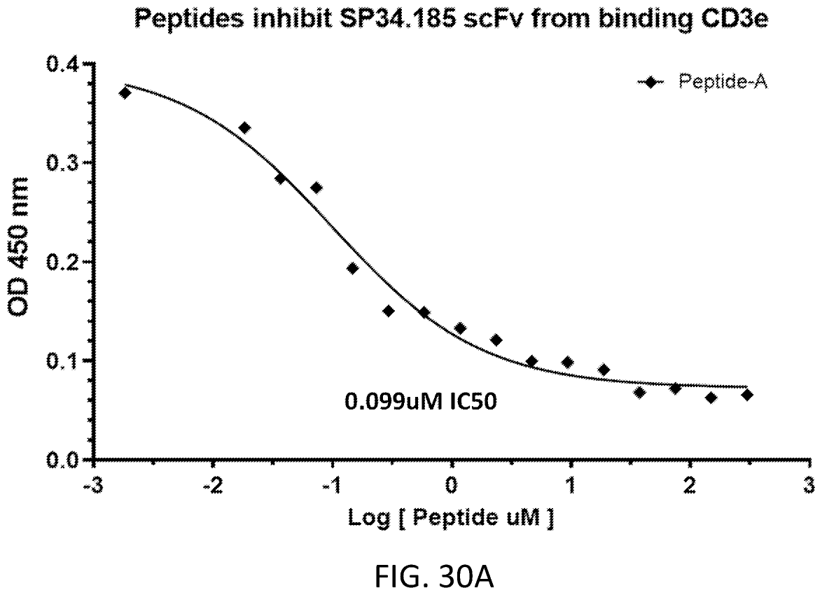

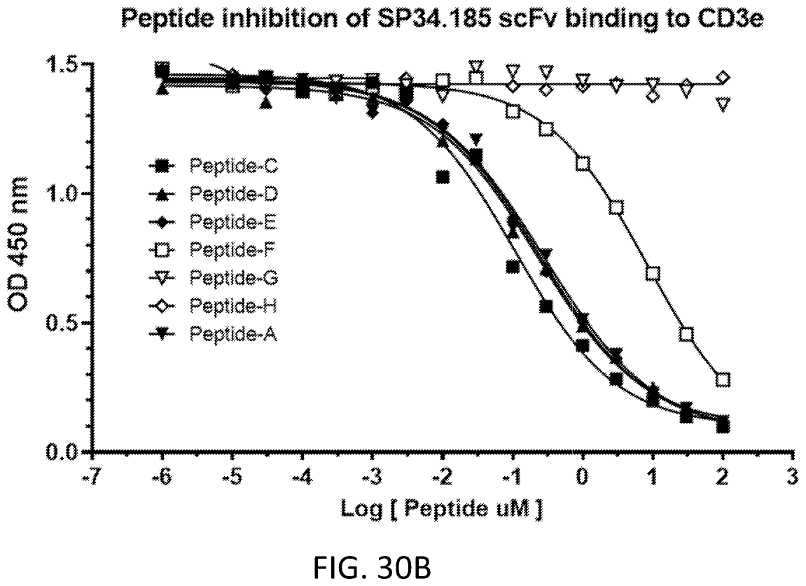

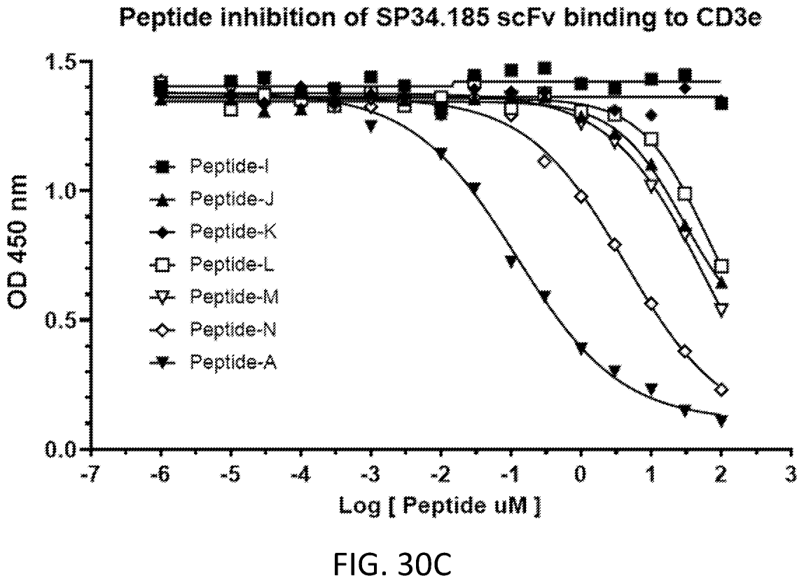

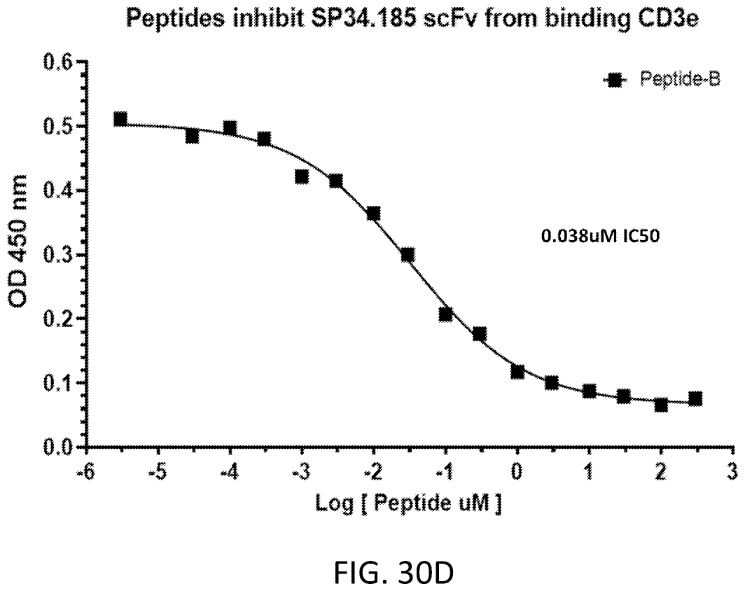

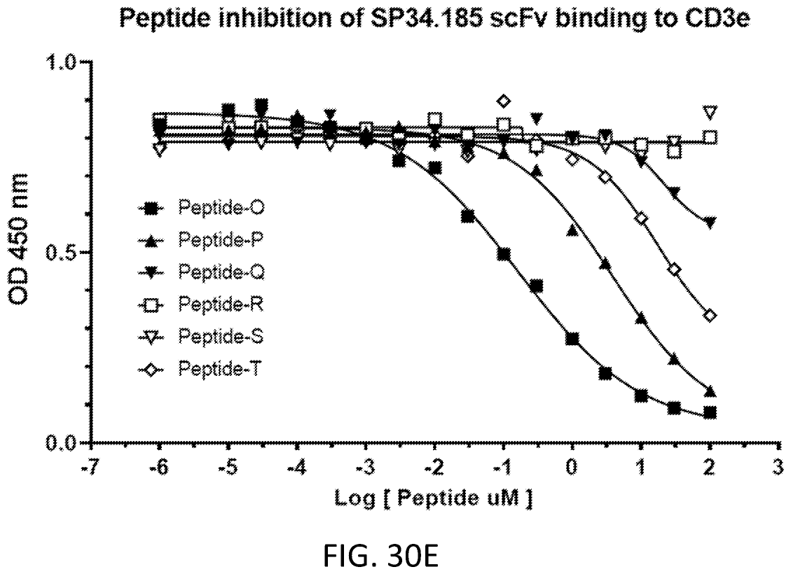

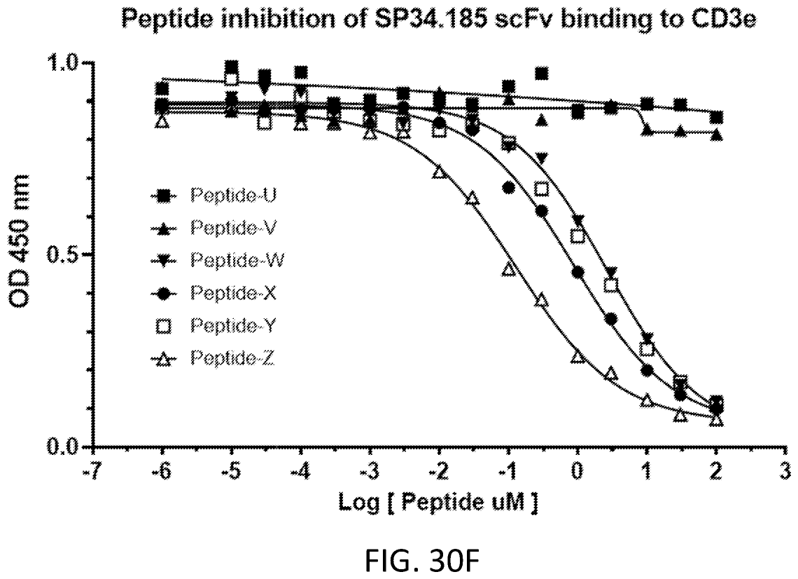

Peptides were evaluated for their ability to inhibit SP34.185 scFv from binding CD3e by standard enzyme linked immunosorbent assays (ELISAs). Briefly, a fixed concentration of SP34.185 scFv was incubated with varying concentrations of peptides in solution. SP34.185scFv and peptide solutions were incubated for 1 hr prior to addition to CD3 coated plates. Binding was allowed to proceed for 30 min prior to washing. After washing, bound SP34.185 scFv using a secondary horse radish peroxidase antibody conjugate. After washing again, plates were developed using standard ELISA techniques and stopped using acid. The concentration of peptide required to inhibit 50% of the SP34.185 scFv CD3 binding signal (IC50) was calculated using Graphpad prism. Data is shown in FIGS. 30A-30F and summarized in Tables 33A-33D.

TABLE 33A
Summary of FIG. 30B

| ELISA | Peptide-A | Peptide-C | Peptide-D | Peptide-E | Peptide-F | Peptide-G | Peptide-H |
|---|---|---|---|---|---|---|---|
| IC50 uM | 0.1926 | 0.1025 | 0.2318 | 0.1905 | 5.484 | >100 | >100 |

TABLE 33B
Summary of FIG. 30C

| ELISA | Peptide-A | Peptide-I | Peptide-10 | Peptide-K | Peptide-L | Peptide-M | Peptide-N |
|---|---|---|---|---|---|---|---|
| IC50 uM | 0.1138 | >100 | 63.18 | >100 | 86.78 | 36.66 | 3.009 |

TABLE 33C
Summary of FIG. 30E

| ELISA | Peptide-O | Peptide-P | Peptide-Q | Peptide-R | Peptide-S | Peptide-T |
|---|---|---|---|---|---|---|
| IC50 uM | 0.1473 | 3.333 | >100 | >100 | >100 | 41.46 |

TABLE 33D
Summary of FIG. 30F

| ELISA | Peptide-U | Peptide-V | Peptide-W | Peptide-X | Peptide-Y | Peptide-Z |
|---|---|---|---|---|---|---|
| IC50 uM | >100 | >100 | 1.912 | 0.6992 | 1.456 | 0.1180 |

TABLE 34
CD3 Ala Scan Sequences-Peptide A and Peptide-B

| | | | SEQ |
|---|---|---|---|
| | anti-CD3 | | ID |
| Peptide-ID | Panned target | Sequence | NO: |
| Peptide-A | SP34.185 | GSQCLGPEWEVCPY | 202 |
| Peptide-C | SP34.185 | ASQCLGPEWEVCPY | 203 |
| Peptide-D | SP34.185 | GAQCLGPEWEVCPY | 204 |
| Peptide-E | SP34.185 | GSACLGPEWEVCPY | 205 |
| Peptide-F | SP34.185 | GSQCAGPEWEVCPY | 206 |
| Peptide-G | SP34.185 | GSQCLAPEWEVCPY | 207 |
| Peptide-H | SP34.185 | GSQCLGAEWEVCPY | 208 |
| Peptide-I | SP34.185 | GSQCLGPAWEVCPY | 209 |
| Peptide-J | SP34.185 | GSQCLGPEAEVCPY | 210 |
| Peptide-K | SP34.185 | GSQCLGPEWAVCPY | 211 |
| Peptide-L | SP34.185 | GSQCLGPEWEACPY | 212 |
| Peptide-M | SP34.185 | GSQCLGPEWEVCAY | 213 |
| Peptide-N | SP34.185 | GSQCLGPEWEVCPA | 214 |
| Peptide-A | SP34.185 | GSQCLGPEWEVCPY | 215 |
| Peptide-B | SP34.185 | VYCGPEFDESVGCM | 216 |
| Peptide-O | SP34.185 | AYCGPEFDESVGCM | 217 |
| Peptide-P | SP34.185 | VACGPEFDESVGCM | 218 |
| Peptide-Q | SP34.185 | VYCAPEFDESVGCM | 219 |
| Peptide-R | SP34.185 | VYCGAEFDESVGCM | 220 |
| Peptide-S | SP34.185 | VYCGPAFDESVGCM | 221 |
| Peptide-T | SP34.185 | VYCGPEADESVGCM | 222 |
| Peptide-U | SP34.185 | VYCGPEFAESVGCM | 223 |
| Peptide-V | SP34.185 | VYCGPEFDASVGCM | 224 |
| Peptide-W | SP34.185 | VYCGPEFDEAVGCM | 225 |
| Peptide-X | SP34.185 | VYCGPEFDESAGCM | 226 |
| Peptide-Y | SP34.185 | VYCGPEFDESVACM | 227 |
| Peptide-Z | SP34.185 | VYCGPEFDESVGCA | 228 |

### Example 17: Panning of the Optimized Phage Library Construction—CD3 scFv Peptides

Once the phage optimization libraries were completed, phage libraries were bio-panned using SP34.185 scFv loaded beads. Multiple rounds of panning were performed where bacteriophage was allowed to bind to SP34.185 scFv loaded beads, washed, eluted, and amplified. Additional selective pressure was included during each round of panning using a fixed concentration of CD3, Peptide-A, or Peptide-B. After panning, phage infected bacteria were plated out and colonies picked into 96 well blocks. Clonal phage was then amplified and separated from bacterial cells via centrifugation. Phage containing supernatants were tested in binding ELISAs against SP34.185 scFv coated plates in the presence or absence of saturating concentration of CD3. Phage able to bind SP34.185 scFv were selected for sequence analysis if the binding signal was reduced in the presence of CD3.

### Example 18: Panning ELISAs—CD3 scFv Peptides

Clonal phages were harvested as crude supernatants and screened via standard enzyme linked immunosorbent assays (ELISAs). Briefly, biotinylated SP34.185 scFv was captured on neutravidin coated plates. Prior to the addition of clonal phage, wells were incubated with blocking buffer and CD3 or blocking buffer alone. Without washing or aspirating, clonal phage supernatants were then added to the wells and incubated for a short time. Wells were then washed followed by detection of bound phage using a horse radish peroxidase conjugated anti-M13 antibody. Clonal phage of interest was then sent for sequence analysis.

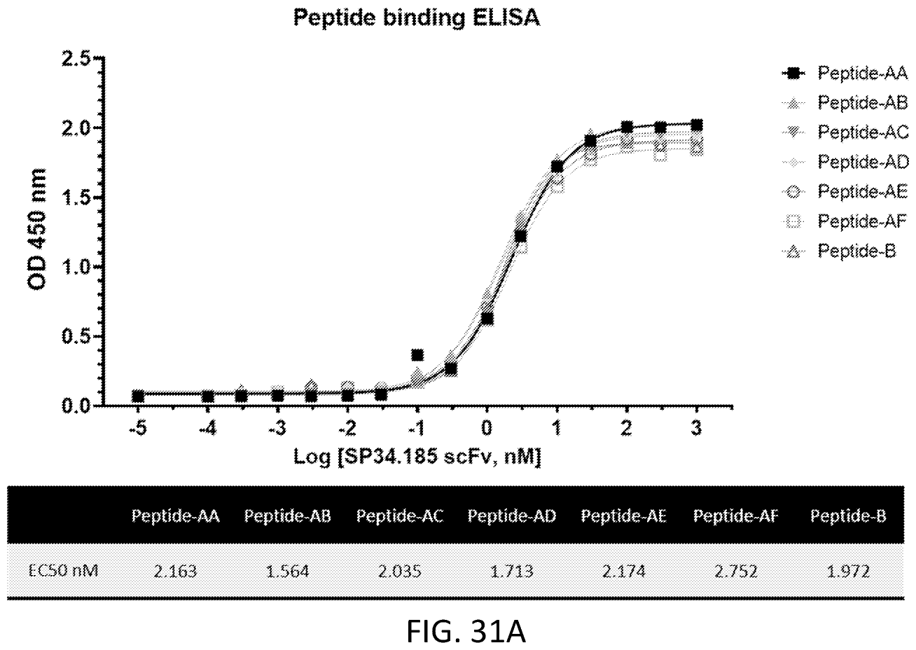

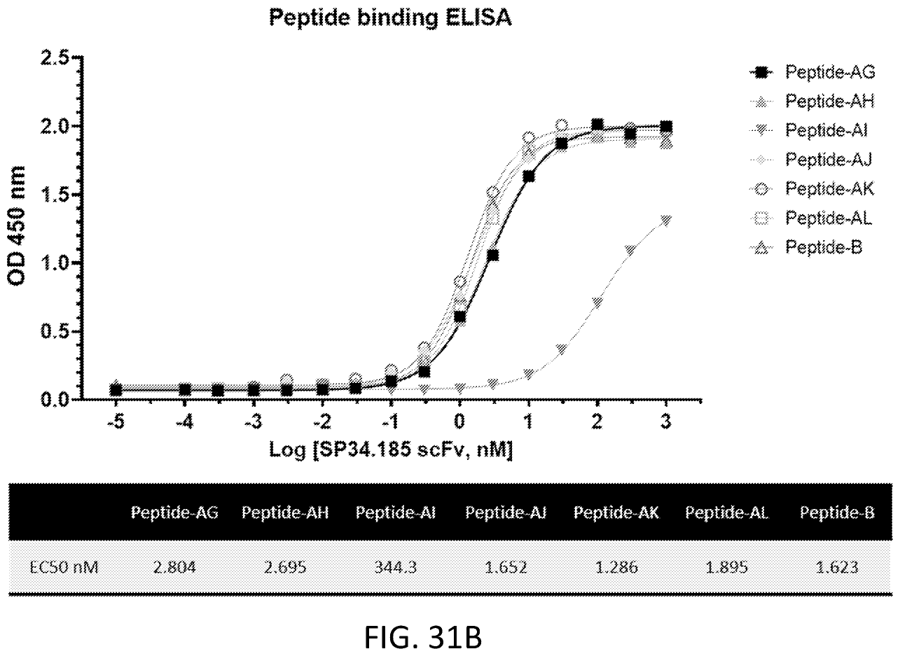

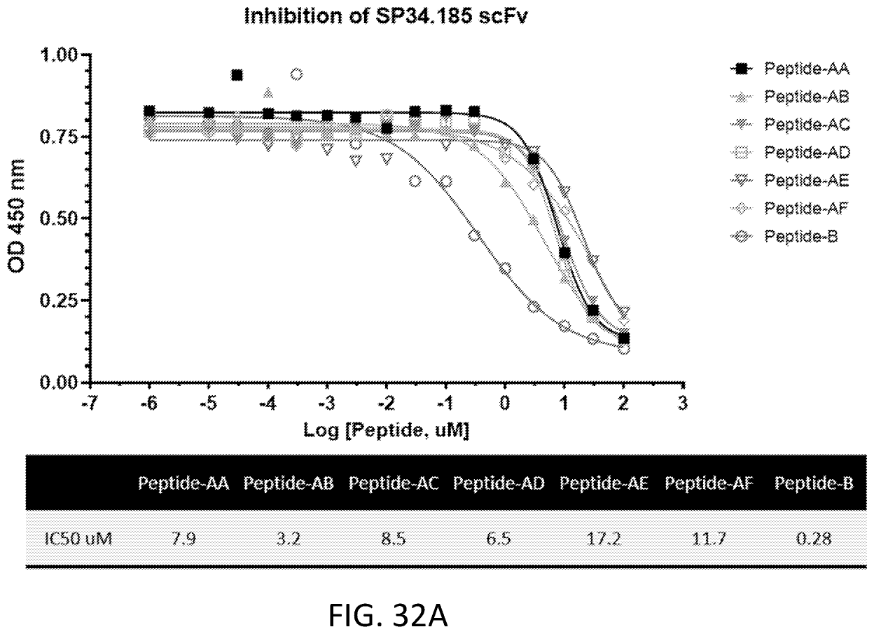

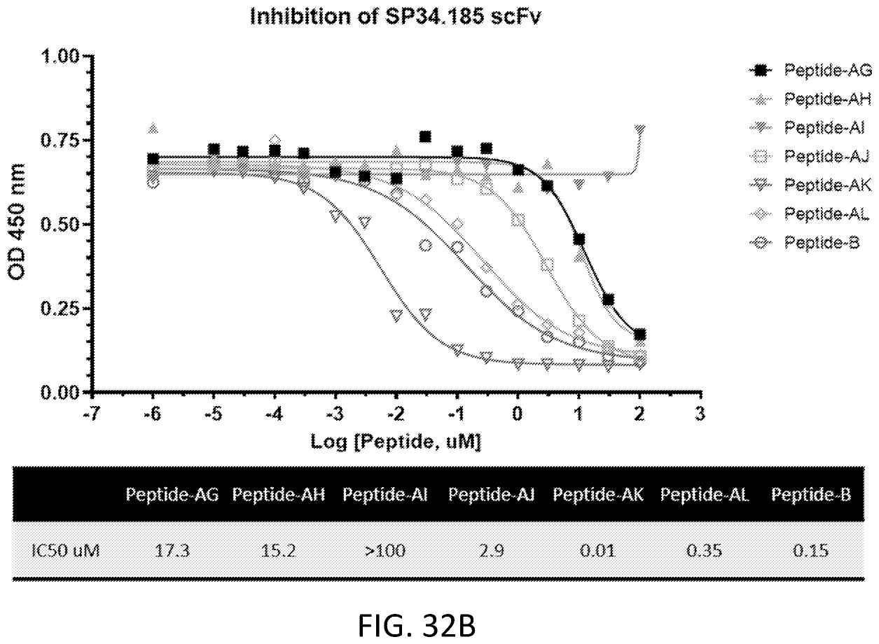

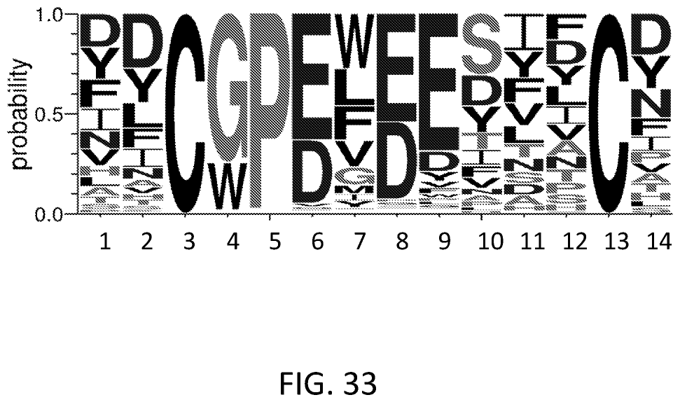

Phage panning results of CD3 scFv Peptide-A library sequences are shown in Table 35. The sequences of those peptides selected for synthesis are shown in Table 36, and further evaluated for binding to anti-CD3 scFv (FIGS. 31A-31B) and inhibition of anti-CD3 scFv binding to CD3 (FIGS. 32A-32B). The consensus sequence shown in FIG. 33 was calculated from all the sequences shown in Table 35 and was generated using WebLogo 3.7.4.

TABLE 35
Clonal Phage Peptide Sequences from the Peptide-B Optimization Library Panning

| | | | | | | | | | | | | | | | Phage binding ELISA | | | |
|---|---|---|---|---|---|---|---|---|---|---|---|---|---|---|---|---|---|---|
| | | | | | | | | | | | | | | | | | SP34.185 | |
| | | | | | | | | | | | | | | | | SP34.185 | scFv signal | SEQ |
| Phage | Amino acid position sequence | | | | | | | | | | | | | | Backgroud | ScFv | in presence | ID |
| ID | 1 | 2 | 3 | 4 | 5 | 6 | 7 | 8 | 9 | 1Q | 11 | 12 | 13 | 14 | signal | signal | of CD3 | NO: |
| Phage-1/ | V | Y | C | G | P | E | F | D | E | S | V | G | C | M | 0.06 | 2.79 | 0.09 | 27 |
| Peptide B | | | | | | | | | | | | | | | | | | |
| Phage-2 | D | D | — | W | — | D | W | E | F | D | F | A | — | A | 0.08 | 2.75 | 0.09 | 229 |
| Phage-3 | Y | I | — | — | L | D | — | P | D | F | L | Y | — | D | 0.08 | 2.88 | 0.10 | 230 |
| Phage-4 | F | D | — | W | — | D | W | E | — | Y | F | V | — | D | 0.08 | 2.79 | 0.09 | 231 |
| Phage-5 | Y | I | — | W | — | D | W | E | — | Y | F | D | — | D | 0.08 | 2.74 | 0.09 | 232 |
| Phage-6 | N | I | — | W | — | D | W | E | D | D | Y | F | — | F | 0.09 | 2.54 | 0.09 | 233 |
| Phage-7 | N | F | — | W | — | D | W | E | Y | I | Y | P | — | I | 0.07 | 2.77 | 0.09 | 234 |
| Phage-8 | — | D | — | W | — | D | W | E | — | D | F | L | — | I | 0.08 | 2.54 | 0.08 | 235 |
| Phage-9 | H | A | — | W | — | D | W | E | — | Y | F | P | — | N | 0.08 | 2.85 | 0.09 | 236 |
| Phage-10 | Y | D | — | — | — | D | V | — | — | — | Y | V | — | V | 0.09 | 2.63 | 0.10 | 237 |
| Phage-11 | I | D | — | W | — | D | W | E | D | D | T | F | — | Y | 0.09 | 2.73 | 0.08 | 238 |
| Phage-12 | Y | L | — | — | — | D | G | — | — | T | L | A | — | Y | 0.08 | 2.66 | 0.15 | 239 |
| Phage-13 | — | D | — | — | — | D | G | — | — | — | I | L | — | Y | 0.11 | 2.13 | 0.08 | 240 |
| Phage-14 | F | I | — | W | — | D | W | E | — | D | Y | F | — | A | 0.07 | 2.44 | 0.09 | 1775 |
| Phage-15 | G | D | — | W | — | D | W | E | W | D | F | Y | — | D | 0.07 | 2.71 | 0.07 | 1776 |
| Phage-16 | Y | L | — | W | — | D | W | E | Y | I | D | L | — | D | 0.12 | 2.67 | 0.08 | 1777 |
| Phage-17 | S | F | — | W | — | D | W | E | — | Y | F | D | — | D | 0.10 | 2.60 | 0.07 | 1778 |
| Phage-18 | D | D | — | W | — | D | W | E | — | Y | A | S | — | D | 0.09 | 2.57 | 0.07 | 1779 |
| Phage-19 | N | L | — | W | — | D | W | E | Y | P | F | F | — | D | 0.09 | 2.52 | 0.09 | 1780 |
| Phage-20 | F | D | — | W | — | D | W | E | — | — | F | V | — | D | 0.08 | 2.34 | 0.09 | 1781 |
| Phage-21 | D | I | — | — | — | D | G | — | — | T | I | I | — | D | 0.13 | 2.30 | 0.10 | 1782 |
| Phage-22 | D | D | — | W | — | D | W | E | Y | Y | A | V | — | D | 0.09 | 2.28 | 0.09 | 1783 |
| Phage-23 | Y | D | — | W | — | D | W | E | — | Y | S | N | — | D | 0.10 | 2.17 | 0.08 | 1784 |
| Phage-24 | I | N | — | W | — | D | W | E | D | Y | F | F | — | D | 0.07 | 2.16 | 0.07 | 1785 |
| Phage-25 | N | I | — | W | — | D | W | E | D | D | T | F | — | F | 0.06 | 2.87 | 0.07 | 1786 |
| Phage-26 | N | I | — | W | — | D | W | E | P | N | S | F | — | F | 0.09 | 2.87 | 0.08 | 1787 |
| Phage-27 | Y | D | — | — | — | — | M | — | — | — | I | D | — | F | 0.09 | 2.39 | 0.08 | 1788 |
| Phage-28 | D | F | — | W | — | D | W | E | F | P | F | I | — | H | 0.11 | 2.73 | 0.12 | 1789 |
| Phage-29 | D | F | — | — | — | — | M | — | — | — | I | T | — | I | 0.07 | 2.36 | 0.08 | 1790 |
| Phage-30 | Y | D | — | — | — | — | — | — | — | — | T | V | — | I | 0.10 | 2.32 | 0.08 | 1791 |
| Phage-31 | H | D | — | W | — | D | W | E | W | D | I | F | — | I | 0.07 | 2.26 | 0.08 | 1792 |
| Phage-32 | H | A | — | W | — | D | W | E | — | Y | N | P | — | N | 0.11 | 2.71 | 0.11 | 1793 |
| Phage-33 | D | V | — | W | — | D | W | E | W | D | F | F | — | N | 0.08 | 2.65 | 0.08 | 1794 |
| Phage-34 | N | — | — | W | — | D | W | E | Y | Y | I | P | — | N | 0.10 | 2.57 | 0.08 | 1795 |
| Phage-35 | I | I | — | W | — | D | W | E | F | I | D | Y | — | N | 0.08 | 2.10 | 0.07 | 1796 |
| Phage-36 | S | L | — | W | — | D | W | E | Y | D | I | A | — | P | 0.07 | 2.53 | 0.08 | 1797 |
| Phage-37 | D | L | — | — | — | — | L | — | — | — | I | F | — | P | 0.08 | 2.49 | 0.09 | 1798 |
| Phage-38 | T | N | — | W | — | D | W | E | W | V | L | P | — | P | 0.14 | 2.47 | 0.10 | 1799 |
| Phage-39 | I | E | — | W | — | D | W | E | P | N | Y | F | — | P | 0.13 | 2.29 | 0.09 | 1800 |
| Phage-40 | I | F | — | W | — | D | W | E | D | Y | — | D | — | P | 0.07 | 2.28 | 0.07 | 1801 |
| Phage-41 | I | D | — | W | — | D | W | E | Y | D | F | F | — | P | 0.07 | 2.26 | 0.08 | 1802 |
| Phage-42 | L | F | — | W | — | D | W | E | D | — | F | F | — | P | 0.18 | 2.11 | 0.13 | 1803 |
| Phage-43 | — | D | — | W | — | D | W | E | D | Y | A | D | — | T | 0.11 | 2.20 | 0.10 | 1804 |
| Phage-44 | — | I | — | W | — | D | W | E | Q | Y | F | P | — | V | 0.11 | 2.34 | 0.09 | 1805 |
| Phage-45 | I | E | — | W | — | D | W | E | P | I | Y | P | — | Y | 0.09 | 2.85 | 0.09 | 1806 |
| Phage-46 | I | T | — | W | — | D | W | E | V | Y | F | P | — | Y | 0.07 | 2.55 | 0.08 | 1807 |
| Phage-47 | I | D | — | W | — | D | W | E | Y | I | H | P | — | Y | 0.06 | 2.51 | 0.09 | 1808 |
| Phage-48 | I | D | — | W | — | D | W | E | Y | I | N | P | — | — | 0.12 | 2.50 | 0.12 | 1809 |
| Phage-49 | A | D | — | W | — | D | W | E | — | A | F | P | — | Y | 0.09 | 2.44 | 0.09 | 1810 |
| Phage-50 | I | D | — | W | — | D | W | E | Y | I | Y | P | — | Y | 0.09 | 2.31 | 0.07 | 1811 |
| Phage-51 | N | I | — | W | — | D | W | E | D | D | N | F | — | F | 0.09 | 2.08 | 0.09 | 1812 |
| Phage-52 | Y | D | — | W | — | D | W | E | Y | V | D | A | — | Y | 0.09 | 2.06 | 0.09 | 1813 |
| Phage-53 | F | — | — | — | — | D | G | — | — | — | Y | V | — | D | 0.09 | 2.03 | 0.11 | 1814 |
| Phage-54 | D | I | — | W | — | D | W | E | Y | I | N | I | — | S | 0.11 | 2.02 | 0.11 | 1815 |
| Phage-55 | F | V | — | W | — | D | W | E | D | F | N | F | — | D | 0.07 | 2.01 | 0.08 | 1816 |
| Phage-56 | F | A | — | W | — | D | W | E | D | Y | — | A | — | D | 0.07 | 2.01 | 0.09 | 1817 |
| Phage-57 | D | N | — | W | — | D | W | E | Y | D | F | F | — | V | 0.08 | 1.99 | 0.09 | 1818 |
| Phage-58 | Y | D | — | W | — | D | W | E | — | Y | N | D | — | A | 0.09 | 1.96 | 0.11 | 1819 |
| Phage-59 | D | D | — | — | — | D | G | — | — | T | I | I | — | V | 0.07 | 1.91 | 0.09 | 1820 |
| Phage-60 | F | P | — | W | — | D | W | E | — | Y | A | I | — | D | 0.10 | 1.89 | 0.10 | 1821 |
| Phage-61 | P | D | — | — | — | D | G | — | — | — | L | F | — | T | 0.12 | 1.86 | 0.07 | 1822 |
| Phage-62 | D | N | — | W | — | D | W | E | Y | D | Y | F | — | V | 0.07 | 1.83 | 0.07 | 1823 |
| Phage-63 | I | F | — | W | — | D | W | E | — | F | Y | D | — | Y | 0.12 | 1.82 | 0.08 | 1824 |
| Phage-64 | A | D | — | W | — | D | W | E | — | Y | F | P | — | N | 0.08 | 1.82 | 0.08 | 1825 |
| Phage-65 | H | T | — | W | — | D | W | E | D | D | I | F | — | N | 0.12 | 1.81 | 0.10 | 1826 |
| Phage-66 | F | A | — | W | — | D | W | E | — | A | F | L | — | L | 0.09 | 1.80 | 0.09 | 1827 |
| Phage-67 | Y | D | — | — | — | — | — | — | — | — | I | A | — | D | 0.08 | 1.77 | 0.08 | 1828 |
| Phage-68 | N | S | — | W | — | D | W | E | Y | D | I | I | — | D | 0.08 | 1.77 | 0.10 | 1829 |
| Phage-69 | F | A | — | W | — | D | W | E | — | V | A | P | — | Y | 0.07 | 1.75 | 0.07 | 1830 |
| Phage-70 | L | D | — | — | — | D | G | — | — | T | L | T | — | Y | 0.10 | 1.75 | 0.12 | 1831 |
| Phage-71 | — | L | — | W | — | D | W | E | — | F | Y | D | — | P | 0.07 | 1.74 | 0.09 | 1832 |
| Phage-72 | H | A | — | W | — | V | W | E | — | Y | F | P | — | N | 0.07 | 1.72 | 0.08 | 1833 |
| Phage-73 | N | E | — | W | — | N | G | E | P | T | F | P | — | T | 0.08 | 1.71 | 0.07 | 1834 |
| Phage-74 | L | T | — | — | — | D | G | — | — | T | L | Y | — | D | 0.08 | 1.70 | 0.07 | 1835 |
| Phage-75 | Y | D | — | — | — | — | Y | — | — | — | — | P | — | I | 0.13 | 1.67 | 0.09 | 1836 |
| Phage-76 | I | E | — | W | — | D | W | E | P | N | S | F | — | D | 0.09 | 1.66 | 0.08 | 1837 |
| Phage-77 | Y | D | — | — | — | — | L | — | — | — | I | H | — | Y | 0.12 | 1.66 | 0.09 | 1838 |
| Phage-78 | I | — | — | — | — | — | — | — | — | — | T | I | — | N | 0.08 | 1.63 | 0.08 | 1839 |
| Phage-79 | I | — | — | — | — | — | V | E | — | A | Y | L | — | Y | 0.09 | 1.62 | 0.10 | 1840 |
| Phage-80 | F | D | — | — | — | D | G | — | — | T | — | Y | — | D | 0.09 | 1.61 | 0.08 | 1841 |
| Phage-81 | I | D | — | — | — | D | G | — | — | T | I | S | — | Y | 0.08 | 1.57 | 0.11 | 1842 |
| Phage-82 | N | — | — | — | — | — | — | — | — | — | S | T | — | L | 0.10 | 1.55 | 0.11 | 1843 |
| Phage-83 | Y | D | — | — | — | D | G | — | — | — | Y | F | — | D | 0.08 | 1.53 | 0.08 | 1844 |
| Phage-84 | N | F | — | W | — | D | W | E | Y | F | N | D | — | N | 0.09 | 1.53 | 0.09 | 1845 |
| Phage-85 | — | L | — | W | — | D | W | E | A | F | F | D | — | D | 0.07 | 1.47 | 0.07 | 1846 |
| Phage-86 | I | — | — | — | — | — | W | E | W | P | — | A | — | N | 0.16 | 1.47 | 0.10 | 1847 |
| Phage-87 | — | F | — | W | — | D | W | E | D | N | F | F | — | N | 0.08 | 1.46 | 0.10 | 1848 |
| Phage-88 | — | V | — | W | — | D | W | E | T | F | F | P | — | D | 0.08 | 1.46 | 0.08 | 1849 |
| Phage-89 | D | N | — | — | — | D | G | — | — | T | Y | I | — | N | 0.10 | 1.45 | 0.09 | 1850 |
| Phage-90 | D | N | — | W | — | D | W | E | Y | N | F | F | — | V | 0.07 | 1.45 | 0.08 | 1851 |
| Phage-91 | F | — | — | — | — | — | V | E | — | D | Y | L | — | I | 0.10 | 1.43 | 0.10 | 1852 |
| Phage-92 | D | N | — | W | — | D | W | E | Y | D | I | F | — | V | 0.07 | 1.43 | 0.07 | 1853 |
| Phage-93 | I | D | — | — | — | — | — | — | — | — | I | A | — | P | 0.08 | 1.42 | 0.08 | 1854 |
| Phage-94 | Y | F | — | — | — | — | V | E | — | Y | T | L | — | F | 0.10 | 1.42 | 0.10 | 1855 |
| Phage-95 | F | — | — | — | — | — | — | — | — | — | A | P | — | N | 0.06 | 1.37 | 0.08 | 1856 |
| Phage-96 | F | D | — | — | — | — | V | E | — | Y | F | Y | — | A | 0.11 | 1.36 | 0.08 | 1857 |
| Phage-97 | D | F | — | W | — | D | W | E | D | F | F | F | — | A | 0.18 | 1.35 | 0.12 | 1858 |
| Phage-98 | F | F | — | — | — | D | G | — | — | T | L | S | — | N | 0.08 | 1.35 | 0.09 | 1859 |
| Phage-99 | F | I | — | — | — | — | — | — | — | — | — | A | — | L | 0.14 | 1.35 | 0.09 | 1860 |
| Phage- | Y | D | — | — | — | — | — | — | — | A | I | — | — | Y | 0.09 | 1.32 | 0.10 | 1861 |
| 100 | | | | | | | | | | | | | | | | | | |
| Phage- | Y | I | — | W | — | D | W | E | — | Y | L | Y | — | P | 0.10 | 1.32 | 0.15 | 1862 |
| 101 | | | | | | | | | | | | | | | | | | |
| Phage- | F | D | — | W | — | D | W | E | — | P | T | T | — | H | 0.08 | 1.31 | 0.08 | 1863 |
| 102 | | | | | | | | | | | | | | | | | | |
| Phage- | Y | D | — | W | — | D | W | E | D | F | P | I | — | D | 0.14 | 1.31 | 0.10 | 1864 |
| 103 | | | | | | | | | | | | | | | | | | |
| Phage- | — | V | — | W | — | D | W | E | Y | I | D | D | — | S | 0.08 | 1.30 | 0.07 | 1865 |
| 104 | | | | | | | | | | | | | | | | | | |
| Phage- | I | N | — | W | — | D | W | E | V | I | S | F | — | D | 0.12 | 1.30 | 0.08 | 1866 |
| 105 | | | | | | | | | | | | | | | | | | |
| Phage- | L | S | — | W | — | D | W | E | — | V | T | P | — | L | 0.10 | 1.29 | 0.10 | 1867 |
| 106 | | | | | | | | | | | | | | | | | | |
| Phage- | F | A | — | W | — | D | W | E | — | V | D | I | — | Y | 0.09 | 1,28 | 0.08 | 1868 |
| 107 | | | | | | | | | | | | | | | | | | |
| Phage- | Y | D | — | — | — | — | M | — | — | — | I | V | — | D | 0.10 | 1.25 | 0.08 | 1869 |
| 108 | | | | | | | | | | | | | | | | | | |
| Phage- | Y | D | — | W | — | D | W | E | V | F | I | V | — | D | 0.06 | 1.25 | 0.07 | 1870 |
| 109 | | | | | | | | | | | | | | | | | | |
| Phage- | D | N | — | W | — | D | W | E | H | N | F | F | — | V | 0.10 | 1.25 | 0.08 | 1871 |
| 110 | | | | | | | | | | | | | | | | | | |
| Phage- | Y | D | — | — | — | D | G | — | — | — | I | Y | — | P | 0.07 | 1.23 | 0.08 | 1872 |
| 111 | | | | | | | | | | | | | | | | | | |
| Phage- | Y | D | — | — | — | — | — | E | F | P | Y | Y | — | F | 0.12 | 1.23 | 0.12 | 1873 |
| 112 | | | | | | | | | | | | | | | | | | |
| Phage- | A | D | — | — | — | — | Y | — | — | — | — | P | — | V | 0.11 | 1.22 | 0.09 | 1874 |
| 113 | | | | | | | | | | | | | | | | | | |
| Phage- | F | L | — | — | — | — | V | E | — | V | H | Y | — | S | 0.08 | 1.22 | 0.10 | 1875 |
| 114 | | | | | | | | | | | | | | | | | | |
| Phage- | T | D | — | W | — | D | W | E | Y | I | T | S | — | S | 0.08 | 1.22 | 0.08 | 1876 |
| 115 | | | | | | | | | | | | | | | | | | |
| Phage- | A | F | — | — | — | — | L | — | — | — | I | T | — | D | 0.09 | 1.21 | 0.09 | 1877 |
| 116 | | | | | | | | | | | | | | | | | | |
| Phage- | N | D | — | W | — | D | W | E | — | Y | F | S | — | Y | 0.09 | 1.19 | 0.09 | 1878 |
| 117 | | | | | | | | | | | | | | | | | | |
| Phage- | F | D | — | — | — | — | W | E | T | V | T | D | — | Y | 0.08 | 1.19 | 0.09 | 1879 |
| 118 | | | | | | | | | | | | | | | | | | |
| Phage- | N | L | — | — | — | — | M | — | — | — | I | I | — | P | 0.13 | 1.19 | 0.11 | 1880 |
| 119 | | | | | | | | | | | | | | | | | | |
| Phage- | D | L | — | — | — | — | M | — | — | — | I | Y | — | D | 0.10 | 1.19 | 0.14 | 1881 |
| 120 | | | | | | | | | | | | | | | | | | |
| Phage- | F | D | — | — | — | D | G | V | — | D | Y | I | — | D | 0.09 | 1.18 | 0.09 | 1882 |
| 121 | | | | | | | | | | | | | | | | | | |
| Phage- | Y | A | — | W | — | D | W | E | — | D | F | A | — | Y | 0.11 | 1.18 | 0.08 | 1883 |
| 122 | | | | | | | | | | | | | | | | | | |
| Phage- | H | D | — | — | — | — | M | — | — | — | I | V | — | V | 0.10 | 1.17 | 0.10 | 1884 |
| 123 | | | | | | | | | | | | | | | | | | |
| Phage- | — | F | — | — | — | — | — | E | F | I | F | L | — | A | 0.07 | 1.17 | 0.08 | 1885 |
| 124 | | | | | | | | | | | | | | | | | | |
| Phage- | Y | D | — | — | — | — | L | — | — | — | I | L | — | D | 0.08 | 1.16 | 0.09 | 1886 |
| 125 | | | | | | | | | | | | | | | | | | |
| Phage- | S | V | — | W | — | D | W | E | — | F | Y | S | — | D | 0.11 | 1.16 | 0.10 | 1887 |
| 126 | | | | | | | | | | | | | | | | | | |
| Phage- | P | — | — | — | — | D | G | — | — | T | A | I | — | T | 0.13 | 1.16 | 0.10 | 1888 |
| 127 | | | | | | | | | | | | | | | | | | |
| Phage- | D | D | — | — | — | — | L | E | W | Y | Y | P | — | Y | 0.09 | 1.16 | 0.08 | 1889 |
| 128 | | | | | | | | | | | | | | | | | | |
| Phage- | F | I | — | — | — | — | — | — | — | — | L | P | — | N | 0.08 | 1.14 | 0.09 | 1890 |
| 129 | | | | | | | | | | | | | | | | | | |
| Phage- | T | D | — | — | — | — | — | — | — | — | L | P | — | D | 0.11 | 1.14 | 0.33 | 1891 |
| 130 | | | | | | | | | | | | | | | | | | |
| Phage- | F | L | — | — | — | — | — | E | — | D | A | P | — | Y | 0.08 | 1.13 | 0.08 | 1892 |
| 131 | | | | | | | | | | | | | | | | | | |
| Phage- | I | F | — | — | — | D | G | — | — | T | H | I | — | H | 0.10 | 1.13 | 0.07 | 1893 |
| 132 | | | | | | | | | | | | | | | | | | |
| Phage- | — | F | — | W | — | D | W | E | Y | I | D | F | — | N | 0.10 | 1.11 | 0.21 | 1894 |
| 133 | | | | | | | | | | | | | | | | | | |
| Phage- | I | F | — | — | — | — | Y | — | — | — | L | H | — | I | 0.12 | 1.11 | 0.11 | 1895 |
| 134 | | | | | | | | | | | | | | | | | | |
| Phage- | H | L | — | W | — | D | W | E | W | Y | — | D | — | P | 0.08 | 1.11 | 0.10 | 1896 |
| 135 | | | | | | | | | | | | | | | | | | |
| Phage- | F | I | — | — | — | — | M | — | — | — | I | A | — | N | 0.08 | 1.11 | 0.09 | 1897 |
| 136 | | | | | | | | | | | | | | | | | | |
| Phage- | I | F | — | — | — | — | V | E | M | I | F | L | — | N | 0.09 | 1.10 | 0.08 | 1898 |
| 137 | | | | | | | | | | | | | | | | | | |
| Phage- | Y | D | — | — | — | — | W | E | F | P | — | D | — | I | 0.11 | 1.09 | 0.11 | 1899 |
| 138 | | | | | | | | | | | | | | | | | | |
| Phage- | N | L | — | — | — | — | L | — | — | — | I | T | — | F | 0.10 | 1.09 | 0.08 | 1900 |
| 139 | | | | | | | | | | | | | | | | | | |
| Phage- | F | — | — | — | — | — | V | E | D | F | Y | F | — | Y | 0.08 | 1.09 | 0.08 | 1901 |
| 140 | | | | | | | | | | | | | | | | | | |
| Phage- | D | — | — | — | — | — | — | — | — | — | L | — | — | N | 0.11 | 1.07 | 0.11 | 1902 |
| 141 | | | | | | | | | | | | | | | | | | |
| Phage- | D | — | — | — | — | — | — | — | — | — | L | P | — | D | 0.08 | 1.07 | 0.08 | 1903 |
| 142 | | | | | | | | | | | | | | | | | | |
| Phage- | A | I | — | — | — | — | L | — | — | — | I | A | — | P | 0.09 | 1.07 | 0.09 | 1904 |
| 143 | | | | | | | | | | | | | | | | | | |
| Phage- | — | I | — | — | — | — | V | E | D | Y | N | L | — | Y | 0.08 | 1.07 | 0.09 | 1905 |
| 144 | | | | | | | | | | | | | | | | | | |
| Phage- | H | T | — | W | — | D | W | E | D | Y | T | V | — | P | 0.10 | 1.06 | 0.09 | 1906 |
| 145 | | | | | | | | | | | | | | | | | | |
| Phage- | S | D | — | W | — | D | W | E | Y | F | Y | D | — | N | 0.10 | 1.06 | 0.08 | 1907 |
| 146 | | | | | | | | | | | | | | | | | | |
| Phage- | — | F | — | — | — | D | G | — | — | T | — | H | — | D | 0.09 | 1.05 | 0.08 | 1908 |
| 147 | | | | | | | | | | | | | | | | | | |
| Phage- | D | — | — | — | — | — | Y | — | — | — | — | H | — | I | 0.09 | 1.05 | 0.08 | 1909 |
| 148 | | | | | | | | | | | | | | | | | | |
| Phage- | A | D | — | — | — | D | G | — | — | — | I | I | — | H | 0.07 | 1.05 | 0.08 | 1910 |
| 149 | | | | | | | | | | | | | | | | | | |
| Phage- | F | — | — | — | — | — | L | — | — | — | L | T | — | V | 0.10 | 1.05 | 0.08 | 1911 |
| 150 | | | | | | | | | | | | | | | | | | |
| Phage- | I | L | — | — | — | — | V | E | — | D | Y | Y | — | Y | 0.11 | 1.04 | 0.09 | 1912 |
| 151 | | | | | | | | | | | | | | | | | | |
| Phage- | H | L | — | W | — | D | W | E | — | Y | H | S | — | D | 0.09 | 1.04 | 0.09 | 1913 |
| 152 | | | | | | | | | | | | | | | | | | |
| Phage- | I | F | — | W | — | D | W | E | D | Y | N | F | — | T | 0.08 | 1.04 | 0.11 | 1914 |
| 153 | | | | | | | | | | | | | | | | | | |
| Phage- | I | V | — | — | — | D | G | — | — | T | L | T | — | H | 0.12 | 1.04 | 0.11 | 1915 |
| 154 | | | | | | | | | | | | | | | | | | |
| Phage- | A | D | — | W | — | D | W | E | W | D | Y | T | — | D | 0.12 | 1.03 | 0.11 | 1916 |
| 155 | | | | | | | | | | | | | | | | | | |
| Phage- | I | T | — | — | — | — | — | — | — | — | T | T | — | N | 0.20 | 1.02 | 0.21 | 1917 |
| 156 | | | | | | | | | | | | | | | | | | |
| Phage- | Y | H | — | W | — | D | W | E | — | Y | T | S | — | D | 0.20 | 1.02 | 0.09 | 1918 |
| 157 | | | | | | | | | | | | | | | | | | |
| Phage- | N | — | — | — | — | — | V | E | — | Y | A | L | — | T | 0.11 | 1.01 | 0.10 | 1919 |
| 158 | | | | | | | | | | | | | | | | | | |
| Phage- | F | I | — | — | — | — | M | — | — | — | I | H | — | D | 0.15 | 1.00 | 0.19 | 1920 |
| 159 | | | | | | | | | | | | | | | | | | |
| Phage- | D | N | — | W | — | D | W | E | — | F | A | V | — | P | 0.14 | 1.00 | 0.10 | 1921 |
| 160 | | | | | | | | | | | | | | | | | | |
| Phage- | Y | D | — | — | — | — | L | — | — | T | — | V | — | D | 0.10 | 1.00 | 0.09 | 1922 |
| 161 | | | | | | | | | | | | | | | | | | |
| Phage- | Y | D | — | — | — | — | — | — | — | — | I | A | — | Y | 0.08 | 0.99 | 0.08 | 1923 |
| 162 | | | | | | | | | | | | | | | | | | |
| Phage- | I | D | — | W | — | D | W | E | Y | T | — | H | — | D | 0.07 | 0.97 | 0.09 | 1924 |
| 163 | | | | | | | | | | | | | | | | | | |
| Phage- | D | D | — | — | — | — | L | — | — | — | I | I | — | I | 0.09 | 0.96 | 0.09 | 1925 |
| 164 | | | | | | | | | | | | | | | | | | |
| Phage- | — | — | — | — | — | — | Y | — | — | — | S | F | — | F | 0.09 | 0.91 | 0.08 | 1926 |
| 165 | | | | | | | | | | | | | | | | | | |
| Phage- | F | N | — | W | — | D | W | E | D | P | Y | F | — | V | 0.09 | 0.86 | 0.07 | 1927 |
| 166 | | | | | | | | | | | | | | | | | | |
| Phage- | Y | D | — | — | — | — | Y | — | — | — | S | Y | — | S | 0.08 | 0.82 | 0.07 | 1928 |
| 167 | | | | | | | | | | | | | | | | | | |
| Phage- | — | A | — | W | — | D | W | E | Y | T | D | S | — | F | 0.13 | 0.79 | 0.09 | 1929 |
| 168 | | | | | | | | | | | | | | | | | | |
| Phage- | T | D | — | — | — | — | — | — | — | — | — | A | — | Y | 0.10 | 0.77 | 0.09 | 1930 |
| 169 | | | | | | | | | | | | | | | | | | |
| Phage- | T | D | — | W | — | D | W | E | F | Y | A | D | — | D | 0.07 | 0.75 | 0.08 | 1931 |
| 170 | | | | | | | | | | | | | | | | | | |
| Phage- | Y | D | — | — | — | — | L | — | — | — | — | I | — | H | 0.09 | 0.69 | 0.09 | 1932 |
| 171 | | | | | | | | | | | | | | | | | | |
| Phage- | S | D | — | — | — | D | G | — | — | — | I | I | — | T | 0.07 | 0.69 | 0.07 | 1933 |
| 172 | | | | | | | | | | | | | | | | | | |
| Phage- | Y | — | — | — | — | — | — | — | — | — | I | D | — | D | 0.08 | 0.67 | 0.09 | 1934 |
| 173 | | | | | | | | | | | | | | | | | | |
| Phage- | F | F | — | — | — | — | I | — | — | — | I | A | — | V | 0.08 | 0.62 | 0.09 | 1935 |
| 174 | | | | | | | | | | | | | | | | | | |
| Phage- | D | — | — | — | — | — | — | — | — | — | T | F | — | D | 0.16 | 0.60 | 0.10 | 1936 |
| 175 | | | | | | | | | | | | | | | | | | |
| Phage- | Y | D | — | — | — | — | W | E | W | P | I | D | — | V | 0.10 | 0.59 | 0.10 | 1937 |
| 176 | | | | | | | | | | | | | | | | | | |
| Phage- | F | — | — | — | — | — | T | E | L | F | S | F | — | Y | 0.13 | 0.59 | 0.11 | 1938 |
| 177 | | | | | | | | | | | | | | | | | | |
| Phage- | Y | — | — | — | — | — | V | — | — | — | I | T | — | P | 0.15 | 0.42 | 0.11 | 1939 |
| 178 | | | | | | | | | | | | | | | | | | |
| Phage- | I | L | — | — | — | — | — | — | — | — | I | N | — | N | 0.09 | 0.37 | 0.25 | 1940 |
| 179 | | | | | | | | | | | | | | | | | | |
| Phage- | — | V | — | — | — | A | M | G | Q | H | Y | L | — | D | 0.08 | 0.09 | 0.08 | 1941 |
| 180 | | | | | | | | | | | | | | | | | | |
| Phage- | — | V | — | — | T | K | M | G | — | H | Y | L | — | S | 0.08 | 0.08 | 0.08 | 1942 |
| 181 | | | | | | | | | | | | | | | | | | |
| Phage- | Y | D | — | W | — | D | W | E | Y | V | Y | A | — | Y | 0.08 | 0.98 | 0.08 | 1943 |
| 182 | | | | | | | | | | | | | | | | | | |
| Phage- | D | L | — | — | — | — | L | — | — | — | — | N | — | D | 0.09 | 0.98 | 0.08 | 1944 |
| 183 | | | | | | | | | | | | | | | | | | |
| Phage- | Y | — | — | — | — | — | — | — | — | — | T | V | — | Y | 0.14 | 0.97 | 0.16 | 1945 |
| 184 | | | | | | | | | | | | | | | | | | |
| Phage- | L | D | — | W | — | D | W | E | W | P | Y | S | — | N | 0.08 | 0.96 | 0.09 | 1946 |
| 185 | | | | | | | | | | | | | | | | | | |
| Phage- | F | I | — | W | — | D | W | E | D | D | F | F | — | Y | 0.08 | 0.96 | 0.09 | 1947 |
| 186 | | | | | | | | | | | | | | | | | | |
| Phage- | D | L | — | — | — | — | V | E | W | Y | F | F | — | N | 0.11 | 0.95 | 0.10 | 1948 |
| 187 | | | | | | | | | | | | | | | | | | |
| Phage- | Y | D | — | — | — | — | L | — | — | — | I | V | — | F | 0.07 | 0.94 | 0.08 | 1949 |
| 188 | | | | | | | | | | | | | | | | | | |
| Phage- | L | N | — | W | — | V | W | E | D | D | — | F | — | Y | 0.09 | 0.92 | 0.09 | 1950 |
| 189 | | | | | | | | | | | | | | | | | | |
| Phage- | F | N | — | W | — | D | W | E | D | P | N | F | — | V | 0.09 | 0.91 | 0.09 | 1951 |
| 190 | | | | | | | | | | | | | | | | | | |
| Phage- | — | I | — | W | — | D | W | E | D | D | Y | F | — | P | 0.10 | 0.91 | 0.13 | 1952 |
| 191 | | | | | | | | | | | | | | | | | | |
| Phage- | F | L | — | — | — | — | — | — | — | — | S | V | — | Y | 0.10 | 0.91 | 0.08 | 1953 |
| 192 | | | | | | | | | | | | | | | | | | |
| Phage- | Y | D | I | — | — | — | L | — | — | — | I | F | — | Y | 0.10 | 0.91 | 0.09 | 1954 |
| 193 | | | | | | | | | | | | | | | | | | |
| Phage- | H | L | — | — | — | D | G | — | — | — | F | T | — | F | 0.11 | 0.90 | 0.10 | 1955 |
| 194 | | | | | | | | | | | | | | | | | | |
| Phage- | Y | F | — | — | — | — | M | — | — | — | L | Y | — | I | 0.08 | 0.90 | 0.08 | 1956 |
| 195 | | | | | | | | | | | | | | | | | | |
| Phage- | Y | — | — | — | — | — | V | E | — | Y | A | N | — | Y | 0.07 | 0.90 | 0.07 | 1957 |
| 196 | | | | | | | | | | | | | | | | | | |
| Phage- | N | T | — | — | — | — | — | — | — | — | T | A | — | Y | 0.16 | 0.90 | 0.58 | 1958 |
| 197 | | | | | | | | | | | | | | | | | | |
| Phage- | I | D | — | W | — | D | W | E | — | A | F | N | — | Y | 0.09 | 0.90 | 0.08 | 1959 |
| 198 | | | | | | | | | | | | | | | | | | |
| Phage- | A | — | — | — | — | — | L | E | — | F | F | L | — | T | 0.09 | 0.89 | 0.08 | 1960 |
| 199 | | | | | | | | | | | | | | | | | | |
| Phage- | I | — | — | — | — | — | V | E | — | V | H | H | — | Y | 0.08 | 0.89 | 0.08 | 1961 |
| 200 | | | | | | | | | | | | | | | | | | |
| Phage- | F | F | — | — | — | — | — | — | — | — | — | A | — | D | 0.10 | 0.89 | 0.11 | 1962 |
| 201 | | | | | | | | | | | | | | | | | | |
| Phage- | Y | D | — | — | — | — | L | — | — | T | I | I | — | Amino acid position sequence | 0.08 | 0.89 | 0.08 | 1963 |
| 202 | | | | | | | | | | | | | | | | | | |
| Phage- | I | L | — | — | — | — | W | E | Y | P | L | D | — | S | 0.09 | 0.89 | 0.10 | 1964 |
| 203 | | | | | | | | | | | | | | | | | | |
| Phage- | F | I | — | — | — | — | — | — | — | — | T | — | — | N | 0.09 | 0.88 | 0.10 | 1965 |
| 204 | | | | | | | | | | | | | | | | | | |
| Phage- | F | — | — | — | — | — | L | — | — | — | — | S | — | D | 0.17 | 0.88 | 0.15 | 1966 |
| 205 | | | | | | | | | | | | | | | | | | |
| Phage- | H | L | — | — | — | — | L | — | — | — | — | T | — | F | 0.10 | 0.87 | 0.10 | 1967 |
| 206 | | | | | | | | | | | | | | | | | | |
| Phage- | L | I | — | — | — | — | V | E | D | Y | S | L | — | H | 0.09 | 0.87 | 0.09 | 1968 |
| 207 | | | | | | | | | | | | | | | | | | |
| Phage- | Y | F | — | — | — | — | M | — | — | — | — | Y | — | D | 0.08 | 0.87 | 0.08 | 1969 |
| 208 | | | | | | | | | | | | | | | | | | |
| Phage- | H | — | — | — | — | — | M | — | — | — | I | Y | — | I | 0.13 | 0.87 | 0.09 | 1970 |
| 209 | | | | | | | | | | | | | | | | | | |
| Phage- | F | D | — | — | — | — | L | — | — | — | I | N | — | D | 0.08 | 0.87 | 0.09 | 1971 |
| 210 | | | | | | | | | | | | | | | | | | |
| Phage- | Y | — | — | — | — | — | V | E | — | Y | I | Y | — | T | 0.07 | 0.87 | 0.08 | 1972 |
| 211 | | | | | | | | | | | | | | | | | | |
| Phage- | L | A | — | W | — | V | R | E | — | I | N | A | — | I | 0.08 | 0.85 | 0.07 | 1973 |
| 212 | | | | | | | | | | | | | | | | | | |
| Phage- | I | D | — | W | — | D | W | E | D | I | T | F | — | D | 0.08 | 0.85 | 0.08 | 1974 |
| 213 | | | | | | | | | | | | | | | | | | |
| Phage- | I | V | — | — | — | — | L | I | — | — | I | T | — | P | 0.11 | 0.85 | 0.15 | 1975 |
| 214 | | | | | | | | | | | | | | | | | | |
| Phage- | F | — | — | — | — | — | — | E | L | P | A | D | — | D | 0.08 | 0.85 | 0.09 | 1976 |
| 215 | | | | | | | | | | | | | | | | | | |
| Phage- | F | D | — | — | — | — | — | — | — | — | N | P | — | F | 0.10 | 0.85 | 0.09 | 1977 |
| 216 | | | | | | | | | | | | | | | | | | |
| Phage- | D | A | — | W | — | D | W | E | — | Y | S | S | — | D | 0.10 | 0.83 | 0.10 | 1978 |
| 217 | | | | | | | | | | | | | | | | | | |
| Phage- | D | H | — | W | — | D | W | E | P | N | Y | F | — | V | 0.08 | 0.83 | 0.09 | 1979 |
| 218 | | | | | | | | | | | | | | | | | | |
| Phage- | D | — | — | W | — | D | W | E | I | N | Y | I | — | F | 0.09 | 0.83 | 0.10 | 1980 |
| 219 | | | | | | | | | | | | | | | | | | |
| Phage- | I | — | — | W | — | D | W | E | Y | V | Y | A | — | N | 0.10 | 0.82 | 0.09 | 1981 |
| 220 | | | | | | | | | | | | | | | | | | |
| Phage- | D | F | — | — | — | — | V | E | — | D | Y | L | — | D | 0.07 | 0.82 | 0.08 | 1982 |
| 221 | | | | | | | | | | | | | | | | | | |
| Phage- | H | D | — | — | — | D | G | R | — | D | Y | D | — | A | 0.11 | 0.82 | 0.09 | 1983 |
| 222 | | | | | | | | | | | | | | | | | | |
| Phage- | L | A | — | W | — | D | W | E | D | D | Y | F | — | V | 0.08 | 0.82 | 0.09 | 1984 |
| 223 | | | | | | | | | | | | | | | | | | |
| Phage- | D | I | — | W | — | D | W | E | D | Y | L | P | — | V | 0.10 | 0.82 | 0.10 | 1985 |
| 224 | | | | | | | | | | | | | | | | | | |
| Phage- | I | L | — | — | — | — | I | E | V | Y | A | L | — | P | 0.08 | 0.81 | 0.10 | 1986 |
| 225 | | | | | | | | | | | | | | | | | | |
| Phage- | I | F | — | — | — | — | W | E | F | — | — | L | — | N | 0.10 | 0.81 | 0.11 | 1987 |
| 226 | | | | | | | | | | | | | | | | | | |
| Phage- | T | — | — | — | — | — | V | E | D | F | S | L | — | V | 0.07 | 0.80 | 0.08 | 1988 |
| 227 | | | | | | | | | | | | | | | | | | |
| Phage- | F | I | — | — | — | — | W | E | F | V | D | A | — | F | 0.11 | 0.80 | 0.09 | 1989 |
| 228 | | | | | | | | | | | | | | | | | | |
| Phage- | F | A | — | W | — | D | W | E | — | D | S | P | — | D | 0.06 | 0.80 | 0.07 | 1990 |
| 229 | | | | | | | | | | | | | | | | | | |
| Phage- | I | L | — | — | — | — | V | E | — | L | I | F | — | P | 0.12 | 0.80 | 0.08 | 1991 |
| 230 | | | | | | | | | | | | | | | | | | |
| Phage- | F | — | — | — | — | — | V | E | — | Y | I | Y | — | Y | 0.08 | 0.80 | 0.08 | 1992 |
| 231 | | | | | | | | | | | | | | | | | | |
| Phage- | D | S | — | — | — | — | L | — | — | — | I | I | — | D | 0.10 | 0.79 | 0.09 | 1993 |
| 232 | | | | | | | | | | | | | | | | | | |
| Phage- | F | L | — | — | — | D | G | — | — | T | S | V | — | D | 0.11 | 0.79 | 0.08 | 1994 |
| 233 | | | | | | | | | | | | | | | | | | |
| Phage- | F | N | — | W | — | N | G | E | P | T | Y | F | — | V | 0.11 | 0.79 | 0.08 | 1995 |
| 234 | | | | | | | | | | | | | | | | | | |
| Phage- | L | A | — | W | — | V | W | E | Y | P | — | T | — | I | 0.09 | 0.78 | 0.09 | 1996 |
| 235 | | | | | | | | | | | | | | | | | | |
| Phage- | D | — | — | — | — | — | V | E | — | D | — | Y | — | Y | 0.09 | 0.78 | 0.09 | 1997 |
| 236 | | | | | | | | | | | | | | | | | | |
| Phage- | I | T | — | W | — | D | W | E | — | Y | A | N | — | T | 0.08 | 0.77 | 0.07 | 1998 |
| 237 | | | | | | | | | | | | | | | | | | |
| Phage- | F | F | — | — | — | D | G | — | — | T | Y | S | — | I | 0.15 | 0.77 | 0.13 | 1999 |
| 238 | | | | | | | | | | | | | | | | | | |
| Phage- | T | D | — | W | — | D | W | E | Y | A | T | S | — | D | 0.09 | 0.76 | 0.09 | 2000 |
| 239 | | | | | | | | | | | | | | | | | | |
| Phage- | F | N | — | — | — | D | G | Y | — | D | Y | L | — | D | 0.10 | 0.76 | 0.11 | 2001 |
| 240 | | | | | | | | | | | | | | | | | | |
| Phage- | Y | D | — | W | — | D | W | E | V | D | F | H | — | P | 0.11 | 0.76 | 0.08 | 2002 |
| 241 | | | | | | | | | | | | | | | | | | |
| Phage- | N | I | — | W | — | D | W | E | D | D | S | F | — | F | 0.08 | 0.76 | 0.08 | 2003 |
| 242 | | | | | | | | | | | | | | | | | | |
| Phage- | A | T | — | — | — | — | — | — | — | — | I | — | — | S | 0.13 | 0.75 | 0.09 | 2004 |
| 243 | | | | | | | | | | | | | | | | | | |
| Phage- | S | — | — | — | — | — | — | — | — | — | T | F | — | D | 0.10 | 0.74 | 0.09 | 2005 |
| 244 | | | | | | | | | | | | | | | | | | |
| Phage- | P | I | — | — | — | — | Y | — | — | — | D | V | — | A | 0.08 | 0.74 | 0.08 | 2006 |
| 245 | | | | | | | | | | | | | | | | | | |
| Phage- | Y | — | — | — | — | D | G | — | — | Y | N | S | — | I | 0.11 | 0.74 | 0.11 | 2007 |
| 246 | | | | | | | | | | | | | | | | | | |
| Phage- | — | D | — | W | — | D | W | E | V | F | I | A | — | D | 0.11 | 0.74 | 0.10 | 2008 |
| 247 | | | | | | | | | | | | | | | | | | |
| Phage- | D | L | — | — | — | — | V | E | — | V | N | L | — | L | 0.12 | 0.74 | 0.10 | 2009 |
| 248 | | | | | | | | | | | | | | | | | | |
| Phage- | F | D | — | — | — | — | M | — | — | — | T | T | — | F | 0.09 | 0.74 | 0.09 | 2010 |
| 249 | | | | | | | | | | | | | | | | | | |
| Phage- | N | F | — | W | — | D | W | E | P | I | Y | F | — | T | 0.13 | 0.74 | 0.14 | 2011 |
| 250 | | | | | | | | | | | | | | | | | | |
| Phage- | — | D | — | — | — | D | G | — | — | — | F | F | — | L | 0.08 | 0.73 | 0.08 | 2012 |
| 251 | | | | | | | | | | | | | | | | | | |
| Phage- | — | D | — | — | — | D | G | — | — | T | A | F | — | I | 0.10 | 0.73 | 0.09 | 2013 |
| 252 | | | | | | | | | | | | | | | | | | |
| Phage- | N | I | — | — | — | — | M | — | — | — | L | V | — | I | 0.10 | 0.73 | 0.09 | 2014 |
| 253 | | | | | | | | | | | | | | | | | | |
| Phage- | I | I | — | — | — | — | — | — | — | — | F | F | — | F | 0.08 | 0.73 | 0.09 | 2015 |
| 254 | | | | | | | | | | | | | | | | | | |
| Phage- | N | F | — | — | — | — | Y | — | — | — | I | S | — | I | 0.10 | 0.73 | 0.38 | 2016 |
| 255 | | | | | | | | | | | | | | | | | | |
| Phage- | H | L | — | — | — | — | I | E | — | A | D | — | — | N | 0.11 | 0.73 | 0.51 | 2017 |
| 256 | | | | | | | | | | | | | | | | | | |
| Phage- | D | — | — | — | — | — | V | E | — | D | Y | L | — | D | 0.08 | 0.72 | 0.09 | 2018 |
| 257 | | | | | | | | | | | | | | | | | | |
| Phage- | D | — | — | — | — | — | L | — | — | — | I | N | — | D | 0.11 | 0.72 | 0.10 | 2019 |
| 258 | | | | | | | | | | | | | | | | | | |
| Phage- | F | — | — | — | — | — | L | — | — | — | L | F | — | V | 0.09 | 0.72 | 0.08 | 2020 |
| 259 | | | | | | | | | | | | | | | | | | |
| Phage- | P | D | — | — | — | — | W | E | F | Y | — | T | — | N | 0.12 | 0.72 | 0.08 | 2021 |
| 260 | | | | | | | | | | | | | | | | | | |
| Phage- | F | D | — | — | — | — | — | E | Y | I | Y | A | — | T | 0.09 | 0.72 | 0.08 | 2022 |
| 261 | | | | | | | | | | | | | | | | | | |
| Phage- | D | — | — | — | — | — | — | — | — | — | S | I | — | N | 0.12 | 0.72 | 0.11 | 2023 |
| 262 | | | | | | | | | | | | | | | | | | |
| Phage- | D | F | — | — | — | — | V | E | — | Y | I | F | — | F | 0.08 | 0.72 | 0.07 | 2024 |
| 263 | | | | | | | | | | | | | | | | | | |
| Phage- | P | V | — | W | — | D | W | E | Y | V | S | S | — | D | 0.08 | 0.71 | 0.08 | 2025 |
| 264 | | | | | | | | | | | | | | | | | | |
| Phage- | Y | I | — | — | — | — | R | — | — | — | N | L | — | L | 0.09 | 0.71 | 0.09 | 2026 |
| 265 | | | | | | | | | | | | | | | | | | |
| Phage- | Y | D | — | — | — | — | L | — | — | — | I | V | — | D | 0.11 | 0.71 | 0.11 | 2027 |
| 266 | | | | | | | | | | | | | | | | | | |
| Phage- | H | D | — | W | — | D | W | E | D | F | Y | F | — | V | 0.09 | 0.71 | 0.08 | 2028 |
| 267 | | | | | | | | | | | | | | | | | | |
| Phage- | H | — | — | — | — | — | Y | — | — | — | I | D | — | Y | 0.12 | 0.71 | 0.10 | 2029 |
| 268 | | | | | | | | | | | | | | | | | | |
| Phage- | L | F | — | — | — | — | M | P | — | D | I | F | — | N | 0.08 | 0.71 | 0.08 | 2030 |
| 269 | | | | | | | | | | | | | | | | | | |
| Phage- | H | D | — | — | — | — | L | E | F | H | Y | A | — | Y | 0.10 | 0.71 | 0.12 | 2031 |
| 270 | | | | | | | | | | | | | | | | | | |
| Phage- | D | F | — | — | — | — | L | — | — | — | — | N | — | F | 0.08 | 0.70 | 0.08 | 2032 |
| 271 | | | | | | | | | | | | | | | | | | |
| Phage- | Y | F | — | — | — | — | L | — | — | — | I | A | — | N | 0.10 | 0.70 | 0.10 | 2033 |
| 272 | | | | | | | | | | | | | | | | | | |
| Phage- | T | D | — | W | — | D | W | E | D | D | T | I | — | D | 0.10 | 0.70 | 0.08 | 2034 |
| 273 | | | | | | | | | | | | | | | | | | |
| Phage- | Y | D | — | — | — | — | L | — | — | — | I | Y | — | F | 0.09 | 0.70 | 0.08 | 2035 |
| 274 | | | | | | | | | | | | | | | | | | |
| Phage- | Y | — | — | W | — | D | W | W | — | Y | — | T | — | D | 0.10 | 0.70 | 0.11 | 2036 |
| 275 | | | | | | | | | | | | | | | | | | |
| Phage- | P | I | — | — | — | — | L | E | — | — | Y | L | — | N | 0.13 | 0.69 | 0.52 | 2037 |
| 276 | | | | | | | | | | | | | | | | | | |
| Phage- | F | D | — | — | — | — | — | — | — | — | I | V | — | Y | 0.12 | 0.69 | 0.11 | 2038 |
| 277 | | | | | | | | | | | | | | | | | | |
| Phage- | — | L | — | — | — | D | G | I | — | F | F | D | — | P | 0.09 | 0.68 | 0.07 | 2039 |
| 278 | | | | | | | | | | | | | | | | | | |
| Phage- | A | — | — | — | — | — | Y | — | — | — | L | T | — | V | 0.07 | 0.68 | 0.07 | 2040 |
| 279 | | | | | | | | | | | | | | | | | | |
| Phage- | D | F | — | — | — | — | L | — | — | — | I | I | — | A | 0.14 | 0.68 | 0.14 | 2041 |
| 280 | | | | | | | | | | | | | | | | | | |
| Phage- | Y | D | — | — | — | — | — | — | — | — | L | D | — | N | 0.08 | 0.68 | 0.08 | 2042 |
| 281 | | | | | | | | | | | | | | | | | | |
| Phage- | A | I | — | — | — | — | — | — | — | — | — | A | — | D | 0.14 | 0.67 | 0.08 | 2043 |
| 282 | | | | | | | | | | | | | | | | | | |
| Phage- | L | L | — | — | — | D | G | V | — | D | F | F | — | D | 0.10 | 0.67 | 0.10 | 2044 |
| 283 | | | | | | | | | | | | | | | | | | |
| Phage- | N | F | — | — | — | — | L | P | — | D | I | F | — | F | 0.12 | 0.66 | 0.13 | 2045 |
| 284 | | | | | | | | | | | | | | | | | | |
| Phage- | F | — | — | — | — | — | V | E | — | V | S | L | — | N | 0.08 | 0.66 | 0.08 | 2046 |
| 285 | | | | | | | | | | | | | | | | | | |
| Phage- | Y | D | — | — | — | D | G | Y | — | A | F | Y | — | H | 0.12 | 0.66 | 0.10 | 2047 |
| 286 | | | | | | | | | | | | | | | | | | |
| Phage- | N | F | — | — | — | — | T | E | F | D | Y | L | — | D | 0.08 | 0.65 | 0.08 | 2048 |
| 287 | | | | | | | | | | | | | | | | | | |
| Phage- | D | — | — | — | — | D | G | V | — | D | F | I | — | N | 0.06 | 0.65 | 0.08 | 2049 |
| 288 | | | | | | | | | | | | | | | | | | |
| Phage- | T | D | — | W | — | D | W | E | Y | I | Y | S | — | S | 0.08 | 0.65 | 0.07 | 2050 |
| 289 | | | | | | | | | | | | | | | | | | |
| Phage- | F | — | — | — | — | — | — | E | — | I | T | N | — | I | 0.14 | 0.65 | 0.11 | 2051 |
| 290 | | | | | | | | | | | | | | | | | | |
| Phage- | F | D | — | W | — | D | W | E | — | — | F | F | — | H | 0.07 | 0.64 | 0.08 | 2052 |
| 291 | | | | | | | | | | | | | | | | | | |
| Phage- | D | F | — | — | — | D | G | — | — | — | — | F | — | P | 0.08 | 0.63 | 0.08 | 2053 |
| 292 | | | | | | | | | | | | | | | | | | |
| Phage- | H | N | — | — | — | — | L | — | — | — | L | V | — | D | 0.13 | 0.63 | 0.09 | 2054 |
| 293 | | | | | | | | | | | | | | | | | | |
| Phage- | T | — | — | — | — | D | G | A | — | D | Y | T | — | D | 0.07 | 0.63 | 0.07 | 2055 |
| 294 | | | | | | | | | | | | | | | | | | |
| Phage- | F | D | — | — | — | — | — | E | F | P | — | I | — | F | 0.08 | 0.62 | 0.08 | 2056 |
| 295 | | | | | | | | | | | | | | | | | | |
| Phage- | Y | N | — | — | — | — | L | — | — | — | — | T | — | D | 0.09 | 0.62 | 0.08 | 2057 |
| 296 | | | | | | | | | | | | | | | | | | |
| Phage- | F | D | — | — | — | — | L | — | — | — | I | H | — | A | 0.07 | 0.62 | 0.08 | 2058 |
| 297 | | | | | | | | | | | | | | | | | | |
| Phage- | D | I | — | — | — | — | V | E | — | Y | F | L | — | F | 0.15 | 0.61 | 0.10 | 2059 |
| 298 | | | | | | | | | | | | | | | | | | |
| Phage- | F | D | — | — | — | — | V | — | — | — | L | T | — | F | 0.10 | 0.61 | 0.09 | 2060 |
| 299 | | | | | | | | | | | | | | | | | | |
| Phage- | F | D | — | — | — | — | I | E | — | F | H | L | — | F | 0.08 | 0.61 | 0.08 | 2061 |
| 300 | | | | | | | | | | | | | | | | | | |
| Phage- | A | — | — | — | — | — | L | — | — | — | I | I | — | D | 0.12 | 0.61 | 0.10 | 2062 |
| 301 | | | | | | | | | | | | | | | | | | |
| Phage- | Y | N | — | — | — | — | L | — | — | — | I | T | — | N | 0.09 | 0.61 | 0.10 | 2063 |
| 302 | | | | | | | | | | | | | | | | | | |
| Phage- | F | D | — | W | — | D | W | E | — | P | — | D | — | L | 0.08 | 0.61 | 0.07 | 2064 |
| 303 | | | | | | | | | | | | | | | | | | |
| Phage- | D | V | — | — | — | — | L | — | — | — | I | L | — | P | 0.08 | 0.60 | 0.08 | 2065 |
| 304 | | | | | | | | | | | | | | | | | | |
| Phage- | N | — | — | — | — | — | L | — | — | — | L | P | — | P | 0.09 | 0.60 | 0.08 | 2066 |
| 305 | | | | | | | | | | | | | | | | | | |
| Phage- | Y | — | — | W | — | D | W | E | Y | D | I | F | — | S | 0.08 | 0.60 | 0.10 | 2067 |
| 306 | | | | | | | | | | | | | | | | | | |
| Phage- | D | D | — | — | — | — | — | — | — | — | T | Y | — | N | 0.08 | 0.60 | 0.09 | 2068 |
| 307 | | | | | | | | | | | | | | | | | | |
| Phage- | F | — | — | — | — | — | — | E | — | V | F | H | — | Y | 0.09 | 0.60 | 0.09 | 2069 |
| 308 | | | | | | | | | | | | | | | | | | |
| Phage- | T | D | — | W | — | D | W | E | — | Y | F | L | — | D | 0.07 | 0.60 | 0.09 | 2070 |
| 309 | | | | | | | | | | | | | | | | | | |
| Phage- | — | — | — | — | — | — | W | E | — | — | Y | L | — | P | 0.09 | 0.60 | 0.09 | 2071 |
| 310 | | | | | | | | | | | | | | | | | | |
| Phage- | D | D | — | I | — | N | G | Y | A | T | F | I | — | Y | 0.06 | 0.59 | 0.08 | 2072 |
| 311 | | | | | | | | | | | | | | | | | | |
| Phage- | F | L | — | — | — | — | I | E | D | D | T | H | — | Y | 0.16 | 0.59 | 0.38 | 2073 |
| 312 | | | | | | | | | | | | | | | | | | |
| Phage- | F | A | — | W | — | D | W | E | — | T | I | P | — | H | 0.08 | 0.59 | 0.08 | 2074 |
| 313 | | | | | | | | | | | | | | | | | | |
| Phage- | — | L | — | — | — | — | — | — | — | — | Y | N | — | Y | 0.10 | 0.58 | 0.08 | 2075 |
| 314 | | | | | | | | | | | | | | | | | | |
| Phage- | Y | D | — | — | — | — | — | — | — | — | I | S | — | I | 0.09 | 0.58 | 0.09 | 2076 |
| 315 | | | | | | | | | | | | | | | | | | |
| Phage- | Y | D | — | — | — | — | — | — | — | — | — | N | — | Y | 0.12 | 0.57 | 0.10 | 2077 |
| 316 | | | | | | | | | | | | | | | | | | |
| Phage- | A | I | — | W | — | D | W | E | — | F | — | D | — | Y | 0.10 | 0.57 | 0.09 | 2078 |
| 317 | | | | | | | | | | | | | | | | | | |
| Phage- | L | T | — | W | — | V | R | E | — | I | F | A | — | D | 0.07 | 0.57 | 0.08 | 2079 |
| 318 | | | | | | | | | | | | | | | | | | |
| Phage- | — | L | — | — | — | — | — | — | — | — | Y | Y | — | N | 0.09 | 0.57 | 0.09 | 2080 |
| 319 | | | | | | | | | | | | | | | | | | |
| Phage- | N | V | — | — | — | — | Y | — | — | — | A | P | — | N | 0.07 | 0.56 | 0.08 | 2081 |
| 320 | | | | | | | | | | | | | | | | | | |
| Phage- | H | D | — | — | — | — | — | — | — | — | I | S | — | V | 0.11 | 0.55 | 0.09 | 2082 |
| 321 | | | | | | | | | | | | | | | | | | |
| Phage- | F | D | — | — | — | — | L | — | — | T | — | D | — | N | 0.12 | 0.55 | 0.41 | 2083 |
| 322 | | | | | | | | | | | | | | | | | | |
| Phage- | Y | F | — | — | — | — | V | E | — | H | F | Y | — | Y | 0.09 | 0.55 | 0.08 | 2084 |
| 323 | | | | | | | | | | | | | | | | | | |
| Phage- | D | — | — | — | — | — | L | — | — | — | I | I | — | H | 0.09 | 0.54 | 0.09 | 2085 |
| 324 | | | | | | | | | | | | | | | | | | |
| Phage- | D | D | — | — | — | — | V | P | — | D | I | T | — | Y | 0.11 | 0.54 | 0.08 | 2086 |
| 325 | | | | | | | | | | | | | | | | | | |
| Phage- | D | N | — | — | — | — | L | — | — | — | — | V | — | D | 0.10 | 0.54 | 0.08 | 2087 |
| 326 | | | | | | | | | | | | | | | | | | |
| Phage- | — | H | — | W | — | D | W | E | P | N | Y | V | — | D | 0.10 | 0.54 | 0.08 | 2088 |
| 327 | | | | | | | | | | | | | | | | | | |
| Phage- | D | — | — | — | — | — | L | — | — | — | L | F | — | L | 0.09 | 0.53 | 0.09 | 2089 |
| 328 | | | | | | | | | | | | | | | | | | |
| Phage- | D | D | — | — | — | — | L | — | — | — | — | V | — | A | 0.08 | 0.53 | 0.11 | 2090 |
| 329 | | | | | | | | | | | | | | | | | | |
| Phage- | A | A | — | — | — | — | L | — | — | — | I | V | — | D | 0.13 | 0.53 | 0.09 | 2091 |
| 330 | | | | | | | | | | | | | | | | | | |
| Phage- | D | F | — | — | — | — | — | E | — | I | N | N | — | F | 0.14 | 0.52 | 0.48 | 2092 |
| 331 | | | | | | | | | | | | | | | | | | |
| Phage- | Y | — | — | — | — | — | — | — | — | — | N | A | — | Y | 0.08 | 0.52 | 0.07 | 2093 |
| 332 | | | | | | | | | | | | | | | | | | |
| Phage- | — | L | — | — | — | — | — | — | — | — | N | S | — | Y | 0.10 | 0.52 | 0.11 | 2094 |
| 333 | | | | | | | | | | | | | | | | | | |
| Phage- | Y | D | — | — | — | — | — | — | — | — | I | D | — | D | 0.09 | 0.52 | 0.09 | 2095 |
| 334 | | | | | | | | | | | | | | | | | | |
| Phage- | D | S | — | — | — | — | — | E | F | Y | Y | V | — | F | 0.11 | 0.52 | 0.14 | 2096 |
| 335 | | | | | | | | | | | | | | | | | | |
| Phage- | Y | I | — | — | — | — | L | — | — | — | L | I | — | H | 0.10 | 0.52 | 0.08 | 2097 |
| 336 | | | | | | | | | | | | | | | | | | |
| Phage- | F | D | — | — | — | — | V | E | — | D | Y | F | — | Y | 0.11 | 0.52 | 0.10 | 2098 |
| 337 | | | | | | | | | | | | | | | | | | |
| Phage- | F | D | — | — | — | — | Y | — | — | — | L | Y | — | F | 0.08 | 0.52 | 0.07 | 2099 |
| 338 | | | | | | | | | | | | | | | | | | |
| Phage- | — | L | — | — | — | D | G | — | — | Y | S | F | — | H | 0.10 | 0.51 | 0.10 | 2100 |
| 339 | | | | | | | | | | | | | | | | | | |
| Phage- | I | P | — | — | — | — | M | — | — | — | — | V | — | N | 0.12 | 0.51 | 0.09 | 2101 |
| 340 | | | | | | | | | | | | | | | | | | |
| Phage- | I | I | — | — | — | D | G | Y | — | D | F | T | — | D | 0.12 | 0.51 | 0.09 | 2102 |
| 341 | | | | | | | | | | | | | | | | | | |
| Phage- | I | F | — | — | — | — | L | — | — | — | I | I | — | Y | 0.11 | 0.51 | 0.08 | 2103 |
| 342 | | | | | | | | | | | | | | | | | | |
| Phage- | — | D | — | — | — | — | — | — | — | — | Y | D | — | Y | 0.18 | 0.51 | 0.18 | 2104 |
| 343 | | | | | | | | | | | | | | | | | | |
| Phage- | L | S | — | — | — | — | M | — | — | — | L | Y | — | D | 0.12 | 0.51 | 0.08 | 2105 |
| 344 | | | | | | | | | | | | | | | | | | |
| Phage- | Y | D | — | W | — | D | W | E | Y | N | I | D | — | T | 0.08 | 0.51 | 0.09 | 2106 |
| 345 | | | | | | | | | | | | | | | | | | |
| Phage- | N | H | — | — | — | D | G | — | — | T | I | V | — | F | 0.09 | 0.51 | 0.08 | 2107 |
| 346 | | | | | | | | | | | | | | | | | | |
| Phage- | N | F | — | — | — | — | L | — | — | — | I | P | — | H | 0.11 | 0.50 | 0.12 | 2108 |
| 347 | | | | | | | | | | | | | | | | | | |
| Phage- | F | H | — | — | — | — | I | E | — | Y | A | L | — | D | 0.08 | 0.50 | 0.09 | 2109 |
| 348 | | | | | | | | | | | | | | | | | | |
| Phage- | — | — | — | — | — | — | V | E | D | Y | N | L | — | Y | 0.07 | 0.50 | 0.08 | 2110 |
| 349 | | | | | | | | | | | | | | | | | | |
| Phage- | — | — | — | — | — | D | G | — | — | L | A | N | — | Y | 0.09 | 0.50 | 0.08 | 2111 |
| 350 | | | | | | | | | | | | | | | | | | |
| Phage- | L | I | — | — | — | V | I | A | — | D | L | P | — | N | 0.17 | 0.50 | 0.26 | 2112 |
| 351 | | | | | | | | | | | | | | | | | | |
| Phage- | D | I | — | — | — | — | I | P | — | D | — | S | — | D | 0.10 | 0.50 | 0.08 | 2113 |
| 352 | | | | | | | | | | | | | | | | | | |
| Phage- | I | — | — | — | — | — | W | E | — | A | D | Y | — | D | 0.11 | 0.50 | 0.45 | 2114 |
| 353 | | | | | | | | | | | | | | | | | | |
| Phage- | I | D | — | W | — | D | W | E | D | D | S | I | — | Y | 0.10 | 0.50 | 0.10 | 2115 |
| 354 | | | | | | | | | | | | | | | | | | |
| Phage- | — | L | — | — | — | — | V | E | D | F | T | L | — | D | 0.09 | 0.50 | 0.11 | 2116 |
| 355 | | | | | | | | | | | | | | | | | | |
| Phage- | L | — | — | — | — | V | I | E | — | I | Y | Y | — | Y | 0.09 | 0.49 | 0.09 | 2117 |
| 356 | | | | | | | | | | | | | | | | | | |
| Phage- | F | F | — | — | — | — | — | E | V | H | S | D | — | N | 0.14 | 0.49 | 0.38 | 2118 |
| 357 | | | | | | | | | | | | | | | | | | |
| Phage- | N | D | — | — | — | — | V | E | L | V | S | D | — | N | 0.10 | 0.49 | 0.08 | 2119 |
| 358 | | | | | | | | | | | | | | | | | | |
| Phage- | D | L | — | — | — | — | L | — | — | — | T | V | — | D | 0.09 | 0.49 | 0.08 | 2120 |
| 359 | | | | | | | | | | | | | | | | | | |
| Phage- | I | P | — | — | — | — | V | E | D | Y | N | L | — | N | 0.08 | 0.49 | 0.08 | 2121 |
| 360 | | | | | | | | | | | | | | | | | | |
| Phage- | Y | — | — | — | — | — | L | E | W | P | — | V | — | N | 0.10 | 0.49 | 0.10 | 2122 |
| 361 | | | | | | | | | | | | | | | | | | |
| Phage- | Y | D | — | — | — | — | L | — | — | — | — | I | — | N | 0.08 | 0.49 | 0.10 | 2123 |
| 362 | | | | | | | | | | | | | | | | | | |
| Phage- | — | — | — | — | — | D | G | — | — | — | F | D | — | A | 0.08 | 0.49 | 0.08 | 2124 |
| 363 | | | | | | | | | | | | | | | | | | |
| Phage- | N | D | — | — | — | — | W | E | D | T | Y | F | — | L | 0.08 | 0.49 | 0.10 | 2125 |
| 364 | | | | | | | | | | | | | | | | | | |
| Phage- | P | — | — | — | — | — | M | E | — | L | S | N | — | S | 0.13 | 0.48 | 0.16 | 2126 |
| 365 | | | | | | | | | | | | | | | | | | |
| Phage- | D | D | — | — | — | — | — | E | V | I | S | D | — | Y | 0.15 | 0.48 | 0.10 | 2127 |
| 366 | | | | | | | | | | | | | | | | | | |
| Phage- | D | L | — | — | — | — | — | P | — | D | — | P | — | D | 0.08 | 0.48 | 0.08 | 2128 |
| 367 | | | | | | | | | | | | | | | | | | |
| Phage- | I | — | — | — | — | — | — | — | — | — | F | V | — | Y | 0.11 | 0.48 | 0.10 | 2129 |
| 368 | | | | | | | | | | | | | | | | | | |
| Phage- | A | — | — | — | — | — | Y | E | V | F | A | D | — | N | 0.10 | 0.48 | 0.11 | 2130 |
| 369 | | | | | | | | | | | | | | | | | | |
| Phage- | I | D | — | — | — | — | Y | — | — | — | — | D | — | L | 0.09 | 0.48 | 0.09 | 2131 |
| 370 | | | | | | | | | | | | | | | | | | |
| Phage- | H | I | — | W | — | D | W | E | — | F | H | D | — | N | 0.07 | 0.48 | 0.08 | 2132 |
| 371 | | | | | | | | | | | | | | | | | | |
| Phage- | Y | D | — | — | — | — | L | — | — | T | I | T | — | L | 0.08 | 0.48 | 0.08 | 2133 |
| 372 | | | | | | | | | | | | | | | | | | |
| Phage- | Y | L | — | — | — | — | L | — | — | T | I | L | — | N | 0.09 | 0.48 | 0.08 | 2134 |
| 373 | | | | | | | | | | | | | | | | | | |
| Phage- | F | F | — | — | — | — | — | E | — | A | F | L | — | F | 0.10 | 0.48 | 0.14 | 2135 |
| 374 | | | | | | | | | | | | | | | | | | |
| Phage- | I | L | — | — | — | — | L | — | — | — | F | T | — | A | 0.07 | 0.47 | 0.08 | 2136 |
| 375 | | | | | | | | | | | | | | | | | | |
| Phage- | F | H | — | — | — | — | V | E | L | Y | T | D | — | N | 0.09 | 0.47 | 0.08 | 2137 |
| 376 | | | | | | | | | | | | | | | | | | |
| Phage- | N | L | — | — | — | — | V | E | — | Y | N | F | — | Y | 0.08 | 0.47 | 0.08 | 2138 |
| 377 | | | | | | | | | | | | | | | | | | |
| Phage- | F | D | — | — | — | — | V | E | — | T | Y | Y | — | F | 0.21 | 0.47 | 0.09 | 2139 |
| 378 | | | | | | | | | | | | | | | | | | |
| Phage- | — | F | — | — | — | — | — | E | — | D | H | Y | — | Y | 0.12 | 0.47 | 0.37 | 2140 |
| 379 | | | | | | | | | | | | | | | | | | |
| Phage- | A | I | — | — | — | — | W | E | V | V | A | D | — | N | 0.11 | 0.47 | 0.11 | 2141 |
| 380 | | | | | | | | | | | | | | | | | | |
| Phage- | F | I | — | W | — | D | W | E | — | D | N | Y | — | N | 0.12 | 0.47 | 0.25 | 2142 |
| 381 | | | | | | | | | | | | | | | | | | |
| Phage- | I | — | — | — | — | — | — | — | — | — | F | I | — | D | 0.08 | 0.47 | 0.10 | 2143 |
| 382 | | | | | | | | | | | | | | | | | | |
| Phage- | N | L | — | — | — | — | V | E | D | V | Y | D | — | H | 0.12 | 0.47 | 0.43 | 2144 |
| 383 | | | | | | | | | | | | | | | | | | |
| Phage- | H | — | — | — | — | — | V | E | — | Y | H | N | — | N | 0.09 | 0.47 | 0.09 | 2145 |
| 384 | | | | | | | | | | | | | | | | | | |
| Phage- | D | I | — | — | — | — | Y | — | — | — | Y | S | — | T | 0.09 | 0.47 | 0.09 | 2146 |
| 385 | | | | | | | | | | | | | | | | | | |
| Phage- | D | — | — | — | — | — | L | — | — | T | L | I | — | A | 0.13 | 0.46 | 0.10 | 2147 |
| 386 | | | | | | | | | | | | | | | | | | |
| Phage- | I | A | — | — | — | — | M | P | — | D | I | D | — | Y | 0.12 | 0.46 | 0.09 | 2148 |
| 387 | | | | | | | | | | | | | | | | | | |
| Phage- | I | D | — | — | — | — | L | — | — | — | I | F | — | D | 0.10 | 0.46 | 0.10 | 2149 |
| 388 | | | | | | | | | | | | | | | | | | |
| Phage- | Y | F | — | — | — | D | V | E | — | D | F | A | — | D | 0.11 | 0.46 | 0.11 | 2150 |
| 389 | | | | | | | | | | | | | | | | | | |
| Phage- | Y | N | — | — | — | — | W | E | Y | A | I | L | — | D | 0.12 | 0.46 | 0.34 | 2151 |
| 390 | | | | | | | | | | | | | | | | | | |
| Phage- | I | — | — | — | — | — | V | E | D | Y | I | V | — | N | 0.14 | 0.46 | 0.22 | 2152 |
| 391 | | | | | | | | | | | | | | | | | | |
| Phage- | Y | D | — | — | — | — | I | — | — | — | T | P | — | A | 0.07 | 0.46 | 0.07 | 2153 |
| 392 | | | | | | | | | | | | | | | | | | |
| Phage- | D | T | — | W | — | D | W | E | H | I | Y | A | — | D | 0.09 | 0.46 | 0.09 | 2154 |
| 393 | | | | | | | | | | | | | | | | | | |
| Phage- | D | I | — | — | — | — | M | — | — | — | — | T | — | N | 0.13 | 0.45 | 0.12 | 2155 |
| 394 | | | | | | | | | | | | | | | | | | |
| Phage- | Y | D | — | W | — | D | W | E | R | Y | F | P | — | I | 0.10 | 0.45 | 0.09 | 2156 |
| 395 | | | | | | | | | | | | | | | | | | |
| Phage- | H | L | — | — | — | — | L | — | — | — | — | A | — | S | 0.13 | 0.45 | 0.11 | 2157 |
| 396 | | | | | | | | | | | | | | | | | | |
| Phage- | Y | D | — | — | — | D | G | — | — | T | T | I | — | A | 0.09 | 0.45 | 0.09 | 2158 |
| 397 | | | | | | | | | | | | | | | | | | |
| Phage- | Y | — | — | — | — | — | Y | E | D | V | L | D | — | F | 0.07 | 0.45 | 0.08 | 2159 |
| 398 | | | | | | | | | | | | | | | | | | |
| Phage- | D | F | — | — | — | — | M | — | — | T | I | S | — | D | 0.14 | 0.45 | 0.10 | 2160 |
| 399 | | | | | | | | | | | | | | | | | | |
| Phage- | I | L | — | — | — | — | L | — | I | — | L | V | — | D | 0.08 | 0.44 | 0.07 | 2161 |
| 400 | | | | | | | | | | | | | | | | | | |
| Phage- | L | I | — | — | — | — | W | E | V | I | T | N | — | D | 0.12 | 0.44 | 0.40 | 2162 |
| 401 | | | | | | | | | | | | | | | | | | |
| Phage- | Y | D | — | — | — | — | — | — | — | Y | F | — | — | P | 0.09 | 0.44 | 0.09 | 2163 |
| 402 | | | | | | | | | | | | | | | | | | |
| Phage- | A | L | — | — | — | — | V | E | V | Y | D | V | — | V | 0.08 | 0.44 | 0.08 | 2164 |
| 403 | | | | | | | | | | | | | | | | | | |
| Phage- | Y | H | — | W | — | D | W | E | D | V | N | F | — | Y | 0.10 | 0.44 | 0.09 | 2165 |
| 404 | | | | | | | | | | | | | | | | | | |
| Phage- | F | L | I | — | — | M | G | G | L | T | F | Y | — | Y | 0.09 | 0.44 | 0.08 | 2166 |
| 405 | | | | | | | | | | | | | | | | | | |
| Phage- | I | I | — | — | — | — | — | — | — | Y | — | — | — | F | 0.09 | 0.43 | 0.19 | 2167 |
| 406 | | | | | | | | | | | | | | | | | | |
| Phage- | F | F | — | — | — | — | M | — | — | — | — | H | — | F | 0.11 | 0.43 | 0.09 | 2168 |
| 407 | | | | | | | | | | | | | | | | | | |
| Phage- | A | F | — | — | — | — | — | — | — | — | L | F | — | A | 0.09 | 0.43 | 0.09 | 2169 |
| 408 | | | | | | | | | | | | | | | | | | |
| Phage- | N | — | — | — | — | D | G | — | — | T | N | I | — | D | 0.08 | 0.43 | 0.08 | 2170 |
| 409 | | | | | | | | | | | | | | | | | | |
| Phage- | Y | L | — | — | — | — | W | E | W | V | H | N | — | L | 0.13 | 0.43 | 0.09 | 2171 |
| 410 | | | | | | | | | | | | | | | | | | |
| Phage- | A | T | — | — | — | D | G | — | — | — | H | I | — | A | 0.08 | 0.43 | 0.09 | 2172 |
| 411 | | | | | | | | | | | | | | | | | | |
| Phage- | — | — | — | — | — | — | V | E | V | L | D | Y | — | D | 0.07 | 0.42 | 0.08 | 2173 |
| 412 | | | | | | | | | | | | | | | | | | |
| Phage- | I | H | — | — | — | — | W | E | F | Y | T | D | — | D | 0.08 | 0.42 | 0.08 | 2174 |
| 413 | | | | | | | | | | | | | | | | | | |
| Phage- | D | D | — | — | — | — | L | — | — | T | — | A | — | D | 0.13 | 0.42 | 0.32 | 2175 |
| 414 | | | | | | | | | | | | | | | | | | |
| Phage- | Y | L | — | — | — | — | — | — | — | — | I | D | — | N | 0.11 | 0.42 | 0.17 | 2176 |
| 415 | | | | | | | | | | | | | | | | | | |
| Phage- | L | L | — | — | — | — | V | E | D | V | F | A | — | Y | 0.09 | 0.42 | 0.09 | 2177 |
| 416 | | | | | | | | | | | | | | | | | | |
| Phage- | Y | D | — | — | — | — | L | — | — | — | L | H | — | D | 0.08 | 0.42 | 0.08 | 2178 |
| 417 | | | | | | | | | | | | | | | | | | |
| Phage- | F | D | — | — | — | — | L | — | — | T | — | N | — | Y | 0.09 | 0.41 | 0.09 | 2179 |
| 418 | | | | | | | | | | | | | | | | | | |
| Phage- | F | A | — | W | — | D | W | E | — | I | N | D | — | H | 0.08 | 0.41 | 0.09 | 2180 |
| 419 | | | | | | | | | | | | | | | | | | |
| Phage- | Y | — | — | — | — | — | Y | E | — | D | I | Y | — | N | 0.09 | 0.41 | 0.09 | 2181 |
| 420 | | | | | | | | | | | | | | | | | | |
| Phage- | N | V | — | — | — | — | V | E | D | Y | T | F | — | Y | 0.09 | 0.40 | 0.08 | 2182 |
| 421 | | | | | | | | | | | | | | | | | | |
| Phage- | A | — | — | — | — | — | L | E | — | Y | D | F | — | T | 0.12 | 0.40 | 0.08 | 2183 |
| 422 | | | | | | | | | | | | | | | | | | |
| Phage- | F | D | — | — | — | — | I | — | — | — | T | I | — | T | 0.08 | 0.40 | 0.09 | 2184 |
| 423 | | | | | | | | | | | | | | | | | | |
| Phage- | N | L | — | — | — | — | L | — | — | T | L | V | — | A | 0.10 | 0.40 | 0.09 | 2185 |
| 424 | | | | | | | | | | | | | | | | | | |
| Phage- | Y | S | — | W | — | D | W | E | — | Y | L | A | — | N | 0.08 | 0.40 | 0.08 | 2186 |
| 425 | | | | | | | | | | | | | | | | | | |
| Phage- | G | I | — | — | — | — | V | E | D | Y | N | Y | — | D | 0.09 | 0.40 | 0.10 | 2187 |
| 426 | | | | | | | | | | | | | | | | | | |
| Phage- | F | F | — | — | — | — | L | — | — | — | — | N | — | H | 0.07 | 0.40 | 0.07 | 2188 |
| 427 | | | | | | | | | | | | | | | | | | |
| Phage- | Y | — | — | — | — | — | Y | E | — | D | F | Y | — | F | 0.11 | 0.40 | 0.09 | 2189 |
| 428 | | | | | | | | | | | | | | | | | | |
| Phage- | L | — | — | — | — | — | Y | — | — | — | T | D | — | Y | 0.11 | 0.40 | 0.10 | 2190 |
| 429 | | | | | | | | | | | | | | | | | | |
| Phage- | D | — | — | — | — | — | V | E | — | D | F | L | — | Y | 0.10 | 0.40 | 0.08 | 2191 |
| 430 | | | | | | | | | | | | | | | | | | |
| Phage- | T | L | — | — | — | — | V | E | L | Y | I | F | — | D | 0.08 | 0.40 | 0.09 | 2192 |
| 431 | | | | | | | | | | | | | | | | | | |
| Phage- | F | — | — | — | — | — | — | E | Q | I | A | D | — | Y | 0.11 | 0.40 | 0.09 | 2193 |
| 432 | | | | | | | | | | | | | | | | | | |
| Phage- | D | D | — | — | — | — | V | E | — | Y | H | L | — | D | 0.11 | 0.40 | 0.18 | 2194 |
| 433 | | | | | | | | | | | | | | | | | | |
| Phage- | D | — | — | — | — | — | L | E | D | V | T | L | — | H | 0.13 | 0.39 | 0.09 | 2195 |
| 434 | | | | | | | | | | | | | | | | | | |
| Phage- | — | L | — | — | — | — | V | E | D | V | N | L | — | Y | 0.09 | 0.39 | 0.08 | 2196 |
| 435 | | | | | | | | | | | | | | | | | | |
| Phage- | D | I | — | — | — | — | L | — | — | T | I | D | — | Y | 0.09 | 0.39 | 0.09 | 2197 |
| 436 | | | | | | | | | | | | | | | | | | |
| Phage- | T | — | — | — | — | — | V | E | — | D | I | N | — | Y | 0.08 | 0.39 | 0.08 | 2198 |
| 437 | | | | | | | | | | | | | | | | | | |
| Phage- | I | V | — | W | — | D | W | E | — | Y | P | N | — | D | 0.08 | 0.39 | 0.08 | 2199 |
| 438 | | | | | | | | | | | | | | | | | | |
| Phage- | S | D | — | — | — | — | L | — | — | — | I | I | — | T | 0.11 | 0.39 | 0.10 | 2200 |
| 439 | | | | | | | | | | | | | | | | | | |
| Phage- | Y | D | — | — | — | — | L | P | — | D | Y | D | — | N | 0.15 | 0.39 | 0.10 | 2201 |
| 440 | | | | | | | | | | | | | | | | | | |
| Phage- | N | L | — | W | — | D | W | E | — | Y | Y | A | — | D | 0.12 | 0.39 | 0.17 | 2202 |
| 441 | | | | | | | | | | | | | | | | | | |
| Phage- | D | D | — | — | — | — | L | — | — | I | L | P | — | H | 0.10 | 0.39 | 0.08 | 2203 |
| 442 | | | | | | | | | | | | | | | | | | |
| Phage- | S | L | — | — | — | D | G | Q | — | D | Y | T | — | F | 0.08 | 0.39 | 0.08 | 2204 |
| 443 | | | | | | | | | | | | | | | | | | |
| Phage- | L | I | — | W | — | D | W | E | — | Y | N | F | — | T | 0.13 | 0.39 | 0.11 | 2205 |
| 444 | | | | | | | | | | | | | | | | | | |
| Phage- | F | H | — | — | — | D | G | — | — | T | — | P | — | I | 0.08 | 0.39 | 0.08 | 2206 |
| 445 | | | | | | | | | | | | | | | | | | |
| Phage- | F | D | — | — | — | — | W | E | W | I | Y | D | — | F | 0.08 | 0.38 | 0.08 | 2207 |
| 446 | | | | | | | | | | | | | | | | | | |
| Phage- | I | — | — | — | — | — | W | — | — | — | L | D | — | D | 0.08 | 0.38 | 0.09 | 2208 |
| 447 | | | | | | | | | | | | | | | | | | |
| Phage- | L | I | — | — | — | — | I | — | — | — | A | S | — | N | 0.10 | 0.38 | 0.11 | 2209 |
| 448 | | | | | | | | | | | | | | | | | | |
| Phage- | T | S | — | W | V | D | W | E | — | F | S | D | — | I | 0.11 | 0.38 | 0.34 | 2210 |
| 449 | | | | | | | | | | | | | | | | | | |
| Phage- | Y | — | — | — | — | — | V | E | — | D | Y | V | — | D | 0.10 | 0.38 | 0.08 | 2211 |
| 450 | | | | | | | | | | | | | | | | | | |
| Phage- | D | D | — | — | — | — | Q | E | F | I | Y | A | — | I | 0.09 | 0.38 | 0.08 | 2212 |
| 451 | | | | | | | | | | | | | | | | | | |
| Phage- | A | D | — | W | — | D | W | E | — | Y | A | D | — | Y | 0.11 | 0.38 | 0.10 | 2213 |
| 452 | | | | | | | | | | | | | | | | | | |
| Phage- | Y | — | — | — | — | — | — | — | — | — | I | H | — | I | 0.08 | 0.38 | 0.07 | 2214 |
| 453 | | | | | | | | | | | | | | | | | | |
| Phage- | S | E | — | W | — | D | W | E | P | F | F | D | — | N | 0.08 | 0.37 | 0.09 | 2215 |
| 454 | | | | | | | | | | | | | | | | | | |
| Phage- | Y | H | — | — | — | — | M | — | — | — | L | I | — | T | 0.07 | 0.37 | 0.07 | 2216 |
| 455 | | | | | | | | | | | | | | | | | | |
| Phage- | S | D | — | W | — | D | W | E | D | A | Y | F | — | I | 0.09 | 0.37 | 0.07 | 2217 |
| 456 | | | | | | | | | | | | | | | | | | |
| Phage- | — | D | — | — | — | — | V | E | — | Y | Y | H | — | D | 0.08 | 0.37 | 0.08 | 2218 |
| 457 | | | | | | | | | | | | | | | | | | |
| Phage- | D | N | — | — | — | — | Y | — | — | — | I | A | — | N | 0.10 | 0.37 | 0.10 | 2219 |
| 458 | | | | | | | | | | | | | | | | | | |
| Phage- | L | — | — | — | — | V | — | E | F | Y | D | Y | — | Y | 0.08 | 0.37 | 0.10 | 2220 |
| 459 | | | | | | | | | | | | | | | | | | |
| Phage- | Y | T | — | — | — | — | M | — | — | — | — | T | — | I | 0.09 | 0.37 | 0.08 | 2221 |
| 460 | | | | | | | | | | | | | | | | | | |
| Phage- | N | F | — | W | — | D | W | E | V | N | S | F | — | D | 0.09 | 0.37 | 0.08 | 2222 |
| 461 | | | | | | | | | | | | | | | | | | |
| Phage- | N | A | — | W | — | D | W | E | Y | I | D | F | — | N | 0.12 | 0.36 | 0.09 | 2223 |
| 462 | | | | | | | | | | | | | | | | | | |
| Phage- | Y | N | — | — | — | — | M | — | — | — | I | F | — | S | 0.09 | 0.36 | 0.07 | 2224 |
| 463 | | | | | | | | | | | | | | | | | | |
| Phage- | L | D | — | — | — | — | L | — | — | — | I | T | — | Y | 0.08 | 0.36 | 0.09 | 2225 |
| 464 | | | | | | | | | | | | | | | | | | |
| Phage- | H | — | — | — | — | — | — | — | — | — | I | N | — | D | 0.11 | 0.36 | 0.08 | 2226 |
| 465 | | | | | | | | | | | | | | | | | | |
| Phage- | I | I | — | — | — | — | L | P | — | D | Y | V | — | T | 0.08 | 0.36 | 0.08 | 2227 |
| 466 | | | | | | | | | | | | | | | | | | |
| Phage- | D | I | — | — | — | — | — | — | — | — | I | D | — | S | 0.08 | 0.36 | 0.08 | 2228 |
| 467 | | | | | | | | | | | | | | | | | | |
| Phage- | P | — | — | — | — | — | — | — | — | — | — | L | — | F | 0.10 | 0.36 | 0.09 | 2229 |
| 468 | | | | | | | | | | | | | | | | | | |
| Phage- | — | F | — | — | — | — | — | — | — | — | — | D | — | Y | 0.07 | 0.36 | 0.08 | 2230 |
| 469 | | | | | | | | | | | | | | | | | | |
| Phage- | Y | D | — | W | — | D | W | E | — | A | L | P | — | A | 0.08 | 0.36 | 0.08 | 2231 |
| 470 | | | | | | | | | | | | | | | | | | |
| Phage- | D | D | — | W | — | D | W | E | D | Y | — | F | — | F | 0.10 | 0.36 | 0.10 | 2232 |
| 471 | | | | | | | | | | | | | | | | | | |
| Phage- | H | F | — | — | — | — | W | E | L | F | S | D | — | Y | 0.11 | 0.36 | 0.11 | 2233 |
| 472 | | | | | | | | | | | | | | | | | | |
| Phage- | I | T | — | W | — | D | W | E | V | N | F | P | — | Y | 0.07 | 0.35 | 0.07 | 2234 |
| 473 | | | | | | | | | | | | | | | | | | |
| Phage- | P | D | — | — | — | — | L | — | — | — | I | T | — | N | 0.20 | 0.35 | 0.16 | 2235 |
| 474 | | | | | | | | | | | | | | | | | | |
| Phage- | N | L | — | W | — | D | W | E | A | F | F | P | — | Y | 0.08 | 0.35 | 0.07 | 2236 |
| 475 | | | | | | | | | | | | | | | | | | |
| Phage- | F | — | — | — | — | — | — | E | Y | I | R | D | — | Y | 0.08 | 0.35 | 0.07 | 2237 |
| 476 | | | | | | | | | | | | | | | | | | |
| Phage- | F | F | — | — | — | — | — | — | — | — | I | I | — | D | 0.09 | 0.35 | 0.10 | 2238 |
| 477 | | | | | | | | | | | | | | | | | | |
| Phage- | — | L | — | — | — | K | G | G | P | T | Y | N | — | S | 0.08 | 0.35 | 0.10 | 2239 |
| 478 | | | | | | | | | | | | | | | | | | |
| Phage- | L | A | — | W | — | V | W | E | — | P | G | H | — | D | 0.11 | 0.35 | 0.10 | 2240 |
| 479 | | | | | | | | | | | | | | | | | | |
| Phage- | D | — | — | — | — | — | V | E | D | V | N | D | — | Y | 0.07 | 0.35 | 0.08 | 2241 |
| 480 | | | | | | | | | | | | | | | | | | |
| Phage- | D | — | — | — | — | — | — | E | — | A | H | Y | — | N | 0.08 | 0,35 | 0.07 | 2242 |
| 481 | | | | | | | | | | | | | | | | | | |
| Phage- | — | L | — | — | — | — | L | — | — | T | L | T | — | T | 0.08 | 0.35 | 0.07 | 2243 |
| 482 | | | | | | | | | | | | | | | | | | |
| Phage- | Y | — | — | — | — | — | I | E | D | Y | N | L | — | N | 0.10 | 0.34 | 0.09 | 2244 |
| 483 | | | | | | | | | | | | | | | | | | |
| Phage- | Y | I | — | — | — | — | V | E | — | Y | Y | N | — | F | 0.13 | 0.34 | 0.14 | 2245 |
| 484 | | | | | | | | | | | | | | | | | | |
| Phage- | D | I | — | — | — | — | L | — | — | — | I | F | — | F | 0.08 | 0.34 | 0.09 | 2246 |
| 485 | | | | | | | | | | | | | | | | | | |
| Phage- | D | I | — | — | — | — | V | E | — | D | Y | L | — | Y | 0.07 | 0.34 | 0.08 | 2247 |
| 486 | | | | | | | | | | | | | | | | | | |
| Phage- | T | L | — | — | — | — | — | E | — | D | A | P | — | I | 0.10 | 0.34 | 0.08 | 2248 |
| 487 | | | | | | | | | | | | | | | | | | |
| Phage- | N | — | — | W | — | D | W | E | Y | I | N | S | — | V | 0.14 | 0.34 | 0.09 | 2249 |
| 488 | | | | | | | | | | | | | | | | | | |
| Phage- | N | D | — | — | — | — | V | E | — | Y | Y | Y | — | T | 0.07 | 0.34 | 0.09 | 2250 |
| 489 | | | | | | | | | | | | | | | | | | |
| Phage- | I | T | — | — | — | — | M | — | — | — | I | D | — | N | 0.08 | 0.34 | 0.08 | 2251 |
| 490 | | | | | | | | | | | | | | | | | | |
| Phage- | Y | — | — | — | — | — | M | A | — | D | L | I | — | D | 0.10 | 0.34 | 0.29 | 2252 |
| 491 | | | | | | | | | | | | | | | | | | |
| Phage- | I | D | — | — | — | — | L | — | — | — | I | V | — | T | 0.11 | 0,33 | 0.09 | 2253 |
| 492 | | | | | | | | | | | | | | | | | | |
| Phage- | H | T | — | W | — | D | W | E | W | D | — | Y | — | D | 0.08 | 0.33 | 0.07 | 2254 |
| 493 | | | | | | | | | | | | | | | | | | |
| Phage- | I | H | — | — | — | — | W | E | L | I | D | D | — | L | 0.08 | 0.33 | 0.10 | 2255 |
| 494 | | | | | | | | | | | | | | | | | | |
| Phage- | Y | T | — | — | — | — | L | — | — | — | I | T | — | T | 0.08 | 0.33 | 0.08 | 2256 |
| 495 | | | | | | | | | | | | | | | | | | |
| Phage- | F | H | — | — | — | — | V | E | — | T | — | Y | — | F | 0.11 | 0.33 | 0.09 | 2257 |
| 496 | | | | | | | | | | | | | | | | | | |
| Phage- | D | — | — | — | — | — | L | — | — | — | L | I | — | N | 0.07 | 0.33 | 0.08 | 2258 |
| 497 | | | | | | | | | | | | | | | | | | |
| Phage- | I | — | — | — | — | — | — | — | — | — | D | Y | — | I | 0.08 | 0.33 | 0.10 | 2259 |
| 498 | | | | | | | | | | | | | | | | | | |
| Phage- | D | L | — | — | — | — | I | E | — | D | L | V | — | T | 0.09 | 0.33 | 0.09 | 2260 |
| 499 | | | | | | | | | | | | | | | | | | |
| Phage- | N | I | — | — | — | — | L | Q | — | D | I | V | — | P | 0.09 | 0.33 | 0.09 | 2261 |
| 500 | | | | | | | | | | | | | | | | | | |
| Phage- | N | N | — | — | — | — | M | — | — | — | I | T | — | Y | 0.08 | 0.33 | 0.08 | 2262 |
| 501 | | | | | | | | | | | | | | | | | | |
| Phage- | H | T | — | — | — | — | L | — | — | — | I | V | — | V | 0.08 | 0.33 | 0.08 | 2263 |
| 502 | | | | | | | | | | | | | | | | | | |
| Phage- | Y | — | — | — | — | — | I | E | D | I | L | V | — | T | 0.12 | 0.33 | 0.23 | 2264 |
| 503 | | | | | | | | | | | | | | | | | | |
| Phage- | N | T | — | — | — | — | — | E | F | V | H | L | — | P | 0.07 | 0.33 | 0.11 | 2265 |
| 504 | | | | | | | | | | | | | | | | | | |
| Phage- | D | I | — | — | — | — | M | — | — | — | T | V | — | D | 0.10 | 0.33 | 0.09 | 2266 |
| 505 | | | | | | | | | | | | | | | | | | |
| Phage- | A | I | — | — | — | — | V | E | I | V | N | Y | — | Y | 0.09 | 0.32 | 0.07 | 2267 |
| 506 | | | | | | | | | | | | | | | | | | |
| Phage- | H | L | — | — | — | — | V | E | D | P | T | A | — | V | 0.10 | 0.32 | 0.28 | 2268 |
| 507 | | | | | | | | | | | | | | | | | | |
| Phage- | A | D | — | — | — | — | L | — | — | — | I | S | — | T | 0.10 | 0.32 | 0.09 | 2269 |
| 508 | | | | | | | | | | | | | | | | | | |
| Phage- | F | D | — | — | — | — | L | — | — | — | — | I | — | D | 0.07 | 0.32 | 0.09 | 2270 |
| 509 | | | | | | | | | | | | | | | | | | |
| Phage- | D | V | — | — | — | — | — | — | — | — | I | D | — | N | 0.10 | 0.32 | 0.08 | 2271 |
| 510 | | | | | | | | | | | | | | | | | | |
| Phage- | Y | L | — | — | — | — | V | E | — | I | S | I | — | F | 0.08 | 0.32 | 0.07 | 2272 |
| 511 | | | | | | | | | | | | | | | | | | |
| Phage- | S | A | — | — | — | — | — | — | — | — | L | H | — | V | 0.10 | 0.31 | 0.30 | 2273 |
| 512 | | | | | | | | | | | | | | | | | | |
| Phage- | H | L | — | W | — | D | W | E | — | D | S | A | — | N | 0.08 | 0.31 | 0,08 | 2274 |
| 513 | | | | | | | | | | | | | | | | | | |
| Phage- | H | T | — | W | — | D | W | E | Y | D | Y | D | — | F | 0.10 | 0.31 | 0.08 | 2275 |
| 514 | | | | | | | | | | | | | | | | | | |
| Phage- | Y | D | — | — | — | — | W | E | — | V | A | L | — | N | 0.10 | 0.31 | 0.10 | 2276 |
| 515 | | | | | | | | | | | | | | | | | | |
| Phage- | T | L | I | — | — | — | I | E | — | Y | I | V | — | Y | 0.09 | 0.31 | 0.23 | 2277 |
| 516 | | | | | | | | | | | | | | | | | | |
| Phage- | S | — | — | — | — | — | — | — | — | — | I | F | — | T | 0.09 | 0.31 | 0.09 | 2278 |
| 517 | | | | | | | | | | | | | | | | | | |
| Phage- | D | I | — | — | — | — | — | — | — | — | L | H | — | Y | 0.08 | 0.31 | 0.08 | 2279 |
| 518 | | | | | | | | | | | | | | | | | | |
| Phage- | L | F | — | — | — | — | — | E | — | A | Y | L | — | I | 0.08 | 0.31 | 0.08 | 2280 |
| 519 | | | | | | | | | | | | | | | | | | |
| Phage- | I | F | — | — | — | — | I | E | — | D | F | V | — | T | 0.10 | 0.31 | 0.10 | 2281 |
| 520 | | | | | | | | | | | | | | | | | | |
| Phage- | D | D | — | — | — | — | W | E | Y | Y | — | A | — | V | 0.08 | 0.31 | 0.08 | 2282 |
| 521 | | | | | | | | | | | | | | | | | | |
| Phage- | D | D | — | — | — | — | L | — | — | T | T | I | — | Y | 0.10 | 0.31 | 0.09 | 2283 |
| 522 | | | | | | | | | | | | | | | | | | |
| Phage- | A | S | — | — | — | — | L | — | — | — | I | A | — | D | 0.10 | 0.31 | 0.09 | 2284 |
| 523 | | | | | | | | | | | | | | | | | | |
| Phage- | Y | L | — | — | — | — | V | E | D | Y | D | Y | — | Y | 0.08 | 0.31 | 0.09 | 2285 |
| 524 | | | | | | | | | | | | | | | | | | |
| Phage- | D | L | — | — | — | — | W | E | — | T | I | F | — | A | 0.12 | 0.30 | 0.09 | 2286 |
| 525 | | | | | | | | | | | | | | | | | | |
| Phage- | D | F | — | — | — | D | G | E | — | F | Y | I | — | P | 0.12 | 0.30 | 0.11 | 2287 |
| 526 | | | | | | | | | | | | | | | | | | |
| Phage- | — | — | — | — | — | — | V | E | — | N | I | L | — | H | 0.15 | 0.30 | 0.17 | 2288 |
| 527 | | | | | | | | | | | | | | | | | | |
| Phage- | D | — | — | — | — | — | V | E | — | N | Y | F | — | F | 0.12 | 0.30 | 0.21 | 2289 |
| 528 | | | | | | | | | | | | | | | | | | |
| Phage- | N | D | — | W | — | D | W | Y | — | F | L | S | — | D | 0.10 | 0.30 | 0.10 | 2290 |
| 529 | | | | | | | | | | | | | | | | | | |
| Phage- | R | D | — | W | — | D | W | E | V | P | Y | F | — | D | 0.08 | 0.30 | 0.09 | 2291 |
| 530 | | | | | | | | | | | | | | | | | | |
| Phage- | N | L | — | — | — | — | V | E | — | A | — | Y | — | Y | 0.10 | 0.30 | 0.13 | 2292 |
| 531 | | | | | | | | | | | | | | | | | | |
| Phage- | F | D | — | W | — | D | G | E | L | N | Y | L | — | T | 0.23 | 0.30 | 0.08 | 2293 |
| 532 | | | | | | | | | | | | | | | | | | |
| Phage- | Y | — | — | — | — | — | V | E | D | V | N | L | — | I | 0.18 | 0.30 | 0.10 | 2294 |
| 533 | | | | | | | | | | | | | | | | | | |
| Phage- | D | N | — | — | — | — | Y | — | — | — | I | T | — | L | 0.11 | 0.29 | 0.09 | 2295 |
| 534 | | | | | | | | | | | | | | | | | | |
| Phage- | L | N | — | W | — | D | W | E | — | D | Y | S | — | N | 0.09 | 0.29 | 0.09 | 2296 |
| 535 | | | | | | | | | | | | | | | | | | |
| Phage- | P | T | — | — | — | — | V | E | — | L | L | S | — | N | 0.12 | 0.29 | 0.26 | 2297 |
| 536 | | | | | | | | | | | | | | | | | | |
| Phage- | D | H | — | — | — | — | V | E | L | I | F | Y | — | H | 0.11 | 0.29 | 0.08 | 2298 |
| 537 | | | | | | | | | | | | | | | | | | |
| Phage- | F | H | — | — | — | — | L | — | — | — | I | F | — | Y | 0.08 | 0.29 | 0.08 | 2299 |
| 538 | | | | | | | | | | | | | | | | | | |
| Phage- | F | D | — | — | — | — | L | E | — | T | — | V | — | P | 0.16 | 0.29 | 0.16 | 2300 |
| 539 | | | | | | | | | | | | | | | | | | |
| Phage- | D | F | — | — | — | — | L | — | — | — | L | P | — | A | 0.08 | 0.29 | 0.08 | 2301 |
| 540 | | | | | | | | | | | | | | | | | | |
| Phage- | D | S | — | — | — | — | L | — | — | — | I | Y | — | D | 0.09 | 0.29 | 0.09 | 2302 |
| 541 | | | | | | | | | | | | | | | | | | |
| Phage- | A | D | — | W | — | D | W | E | — | F | L | L | — | F | 0.12 | 0.29 | 0.10 | 2303 |
| 542 | | | | | | | | | | | | | | | | | | |
| Phage- | I | L | — | — | — | — | V | E | — | L | D | F | — | N | 0.10 | 0.28 | 0.08 | 2304 |
| 543 | | | | | | | | | | | | | | | | | | |
| Phage- | F | F | — | — | — | — | — | E | — | I | F | L | — | Y | 0.09 | 0.28 | 0.07 | 2305 |
| 544 | | | | | | | | | | | | | | | | | | |
| Phage- | Y | N | — | — | — | D | G | — | — | — | Y | D | — | H | 0.07 | 0.28 | 0.07 | 2306 |
| 545 | | | | | | | | | | | | | | | | | | |
| Phage- | D | N | — | — | — | — | L | — | — | T | I | T | — | F | 0.11 | 0.28 | 0.11 | 2307 |
| 546 | | | | | | | | | | | | | | | | | | |
| Phage- | H | N | — | — | — | D | G | — | — | A | F | I | — | N | 0.09 | 0.28 | 0.23 | 2308 |
| 547 | | | | | | | | | | | | | | | | | | |
| Phage- | D | — | — | — | — | — | V | E | — | D | L | V | — | P | 0.10 | 0.28 | 0.23 | 2309 |
| 548 | | | | | | | | | | | | | | | | | | |
| Phage- | D | — | — | — | — | — | L | — | — | — | L | N | — | F | 0.08 | 0.28 | 0.07 | 2310 |
| 549 | | | | | | | | | | | | | | | | | | |
| Phage- | D | — | — | — | — | — | — | Q | — | D | F | H | — | H | 0.11 | 0.28 | 0.27 | 2311 |
| 550 | | | | | | | | | | | | | | | | | | |
| Phage- | Y | D | — | — | — | — | W | E | F | T | D | D | — | I | 0.10 | 0.28 | 0.18 | 2312 |
| 551 | | | | | | | | | | | | | | | | | | |
| Phage- | D | I | — | — | — | — | Y | E | — | D | I | I | — | Y | 0.14 | 0.28 | 0.19 | 2313 |
| 552 | | | | | | | | | | | | | | | | | | |
| Phage- | F | D | — | — | — | — | L | — | — | T | — | P | — | P | 0.11 | 0.28 | 0.08 | 2314 |
| 553 | | | | | | | | | | | | | | | | | | |
| Phage- | N | — | — | — | — | — | L | — | — | T | S | V | — | D | 0.09 | 0.28 | 0.26 | 2315 |
| 554 | | | | | | | | | | | | | | | | | | |
| Phage- | Y | — | — | — | — | — | W | E | F | — | F | D | — | D | 0.17 | 0.28 | 0.11 | 2316 |
| 555 | | | | | | | | | | | | | | | | | | |
| Phage- | Y | A | — | — | — | — | L | — | — | — | — | T | — | D | 0.09 | 0.28 | 0.08 | 2317 |
| 556 | | | | | | | | | | | | | | | | | | |
| Phage- | F | L | — | — | — | — | V | E | Q | D | Y | F | — | V | 0.08 | 0.28 | 0.10 | 2318 |
| 557 | | | | | | | | | | | | | | | | | | |
| Phage- | D | N | — | — | — | — | — | — | — | — | — | R | — | D | 0.09 | 0.27 | 0.26 | 2319 |
| 558 | | | | | | | | | | | | | | | | | | |
| Phage- | N | D | — | — | — | D | G | I | — | T | — | D | — | Y | 0.10 | 0.27 | 0.24 | 2320 |
| 559 | | | | | | | | | | | | | | | | | | |
| Phage- | Y | F | — | — | — | — | V | E | D | Y | N | D | — | F | 0.08 | 0.27 | 0.09 | 2321 |
| 560 | | | | | | | | | | | | | | | | | | |
| Phage- | N | L | — | — | — | — | — | — | — | — | I | F | — | Y | 0.10 | 0.27 | 0.07 | 2322 |
| 561 | | | | | | | | | | | | | | | | | | |
| Phage- | I | D | — | W | — | D | W | E | — | Y | I | P | — | T | 0.10 | 0.27 | 0.08 | 2323 |
| 562 | | | | | | | | | | | | | | | | | | |
| Phage- | I | — | — | — | — | D | G | — | — | — | F | I | — | A | 0.07 | 0.27 | 0.08 | 2324 |
| 563 | | | | | | | | | | | | | | | | | | |
| Phage- | D | — | — | — | — | — | — | — | — | — | — | V | — | Y | 0.08 | 0.27 | 0.07 | 2325 |
| 564 | | | | | | | | | | | | | | | | | | |
| Phage- | — | F | — | — | — | — | W | E | D | I | T | D | — | D | 0.13 | 0.27 | 0.09 | 2326 |
| 565 | | | | | | | | | | | | | | | | | | |
| Phage- | L | D | — | — | — | — | V | — | — | T | F | T | — | H | 0.08 | 0.26 | 0.08 | 2327 |
| 566 | | | | | | | | | | | | | | | | | | |
| Phage- | D | D | — | — | — | — | Y | — | — | — | F | A | — | H | 0.13 | 0.26 | 0.10 | 2328 |
| 567 | | | | | | | | | | | | | | | | | | |
| Phage- | I | — | — | — | — | — | Y | Q | — | D | L | P | — | N | 0.12 | 0.26 | 0.11 | 2329 |
| 568 | | | | | | | | | | | | | | | | | | |
| Phage- | L | D | — | — | — | — | V | E | — | Y | N | Y | — | V | 0.09 | 0.26 | 0.08 | 2330 |
| 569 | | | | | | | | | | | | | | | | | | |
| Phage- | Y | V | — | — | — | — | — | — | — | — | S | A | — | N | 0.14 | 0.26 | 0.08 | 2331 |
| 570 | | | | | | | | | | | | | | | | | | |
| Phage- | T | P | — | — | — | — | L | E | — | A | I | — | — | Y | 0.10 | 0.26 | 0.10 | 2332 |
| 571 | | | | | | | | | | | | | | | | | | |
| Phage- | Y | F | — | — | — | — | — | — | — | — | A | D | — | N | 0.08 | 0.26 | 0.08 | 2333 |
| 572 | | | | | | | | | | | | | | | | | | |
| Phage- | D | D | — | — | — | — | — | E | — | D | I | I | — | D | 0.12 | 0.26 | 0.25 | 2334 |
| 573 | | | | | | | | | | | | | | | | | | |
| Phage- | L | — | — | — | — | V | V | E | — | L | N | H | — | N | 0.08 | 0.26 | 0.09 | 2335 |
| 574 | | | | | | | | | | | | | | | | | | |
| Phage- | — | I | — | — | — | D | G | E | — | L | I | A | — | A | 0.09 | 0.26 | 0.27 | 2336 |
| 575 | | | | | | | | | | | | | | | | | | |
| Phage- | F | A | — | W | — | D | W | Q | — | T | Y | V | — | N | 0.09 | 0.25 | 0.08 | 2337 |
| 576 | | | | | | | | | | | | | | | | | | |
| Phage- | Y | I | — | — | — | — | V | E | F | L | F | F | — | N | 0.08 | 0.25 | 0.08 | 2338 |
| 577 | | | | | | | | | | | | | | | | | | |
| Phage- | T | Q | — | — | — | K | G | E | P | T | Y | H | — | Y | 0.12 | 0.25 | 0.12 | 2339 |
| 578 | | | | | | | | | | | | | | | | | | |
| Phage- | N | — | — | — | — | — | V | E | — | Y | H | N | — | D | 0.10 | 0.25 | 0.17 | 2340 |
| 579 | | | | | | | | | | | | | | | | | | |
| Phage- | — | — | — | — | — | — | W | E | F | F | S | D | — | A | 0.08 | 0.25 | 0.07 | 2341 |
| 580 | | | | | | | | | | | | | | | | | | |
| Phage- | F | — | — | — | — | — | L | E | — | — | F | F | — | Y | 0.10 | 0.25 | 0.22 | 2342 |
| 581 | | | | | | | | | | | | | | | | | | |
| Phage- | D | — | — | — | = | — | I | E | — | N | F | Y | — | Y | 0.13 | 0.25 | 0.09 | 2343 |
| 582 | | | | | | | | | | | | | | | | | | |
| Phage- | Y | A | — | — | — | — | V | E | — | Y | — | Y | — | A | 0.08 | 0.25 | 0.09 | 2344 |
| 583 | | | | | | | | | | | | | | | | | | |
| Phage- | — | D | — | — | — | — | L | — | — | — | I | I | — | D | 0.08 | 0.25 | 0.07 | 2345 |
| 584 | | | | | | | | | | | | | | | | | | |
| Phage- | N | D | — | — | — | — | M | — | — | — | I | A | — | Y | 0.07 | 0.25 | 0.07 | 2346 |
| 585 | | | | | | | | | | | | | | | | | | |
| Phage- | — | — | — | — | — | — | — | — | — | Y | L | A | — | A | 0.09 | 0.25 | 0.21 | 2347 |
| 586 | | | | | | | | | | | | | | | | | | |
| Phage- | — | I | — | — | — | | — | — | — | — | A | N | — | D | 0.08 | 0.25 | 0.09 | 2348 |
| 587 | | | | | | | | | | | | | | | | | | |
| Phage- | L | T | — | W | — | D | W | E | — | D | F | F | — | N | 0.07 | 0.24 | 0.07 | 2349 |
| 588 | | | | | | | | | | | | | | | | | | |
| Phage- | F | — | — | — | — | — | Q | E | — | I | N | Y | — | Y | 0.10 | 0.24 | 0.23 | 2350 |
| 589 | | | | | | | | | | | | | | | | | | |
| Phage- | T | — | — | — | — | — | L | E | — | F | F | L | — | Y | 0.13 | 0.24 | 0.08 | 2351 |
| 590 | | | | | | | | | | | | | | | | | | |
| Phage- | P | D | — | — | — | — | L | — | — | — | — | A | — | H | 0.12 | 0.24 | 0.09 | 2352 |
| 591 | | | | | | | | | | | | | | | | | | |
| Phage- | N | D | — | — | — | — | — | E | — | I | I | F | — | V | 0.09 | 0.24 | 0.24 | 2353 |
| 592 | | | | | | | | | | | | | | | | | | |
| Phage- | Y | I | — | — | — | — | — | — | — | — | F | Y | — | N | 0.25 | 0.24 | 0.08 | 2354 |
| 593 | | | | | | | | | | | | | | | | | | |
| Phage- | H | A | — | — | — | — | L | — | — | — | L | L | — | N | 0.20 | 0.24 | 0.07 | 2355 |
| 594 | | | | | | | | | | | | | | | | | | |
| Phage- | Y | — | — | — | — | — | W | E | — | A | — | L | — | A | 0.09 | 0.24 | 0.21 | 2356 |
| 595 | | | | | | | | | | | | | | | | | | |
| Phage- | A | F | — | — | — | — | V | E | — | Y | D | L | — | N | 0.10 | 0.24 | 0.16 | 2357 |
| 596 | | | | | | | | | | | | | | | | | | |
| Phage- | Y | N | — | — | — | — | — | — | — | — | — | A | — | S | 0.15 | 0.23 | 0.09 | 2358 |
| 597 | | | | | | | | | | | | | | | | | | |
| Phage- | Y | L | — | — | — | — | V | E | D | D | T | L | — | A | 0.08 | 0.23 | 0.09 | 2359 |
| 598 | | | | | | | | | | | | | | | | | | |
| Phage- | A | V | — | — | — | — | — | — | — | — | — | N | — | D | 0.08 | 0.23 | 0.08 | 2360 |
| 599 | | | | | | | | | | | | | | | | | | |
| Phage- | N | — | — | — | — | — | W | E | V | Y | S | L | — | P | 0.13 | 0.23 | 0.08 | 2361 |
| 600 | | | | | | | | | | | | | | | | | | |
| Phage- | D | F | — | — | — | — | V | E | — | D | T | Y | — | H | 0.07 | 0.23 | 0.07 | 2362 |
| 601 | | | | | | | | | | | | | | | | | | |
| Phage- | I | S | — | — | — | — | Y | E | W | D | Y | A | — | N | 0.08 | 0.23 | 0.10 | 2363 |
| 602 | | | | | | | | | | | | | | | | | | |
| Phage- | — | N | — | — | — | — | L | — | — | — | I | I | — | Y | 0.08 | 0.23 | 0.08 | 2364 |
| 603 | | | | | | | | | | | | | | | | | | |
| Phage- | Y | D | — | — | — | — | — | — | — | T | A | P | — | Y | 0.07 | 0.23 | 0.08 | 2365 |
| 604 | | | | | | | | | | | | | | | | | | |
| Phage- | Y | L | — | — | — | — | — | E | — | N | F | L | — | T | 0.09 | 0.23 | 0.22 | 2366 |
| 605 | | | | | | | | | | | | | | | | | | |
| Phage- | F | D | — | — | — | — | V | — | — | — | — | D | — | A | 0.08 | 0.22 | 0.09 | 2367 |
| 606 | | | | | | | | | | | | | | | | | | |
| Phage- | Y | D | — | — | — | — | Q | E | — | I | S | F | — | N | 0.09 | 0.22 | 0.09 | 2368 |
| 607 | | | | | | | | | | | | | | | | | | |
| Phage- | I | D | — | — | — | — | I | E | L | Y | D | D | — | F | 0.09 | 0.22 | 0.09 | 2369 |
| 608 | | | | | | | | | | | | | | | | | | |
| Phage- | T | F | — | — | — | — | L | — | — | — | — | Y | — | Y | 0.08 | 0.22 | 0.07 | 2370 |
| 609 | | | | | | | | | | | | | | | | | | |
| Phage- | F | F | — | — | — | — | I | — | — | — | N | A | — | V | 0.09 | 0.22 | 0.07 | 2371 |
| 610 | | | | | | | | | | | | | | | | | | |
| Phage- | Y | H | — | W | — | D | W | E | P | I | Y | I | — | I | 0.12 | 0.22 | 0.10 | 2372 |
| 611 | | | | | | | | | | | | | | | | | | |
| Phage- | A | I | — | — | — | — | Y | E | — | D | H | Y | — | Y | 0.08 | 0.22 | 0.08 | 2373 |
| 612 | | | | | | | | | | | | | | | | | | |
| Phage- | P | L | — | — | — | D | G | F | — | N | Y | N | — | F | 0.12 | 0.22 | 0.08 | 2374 |
| 613 | | | | | | | | | | | | | | | | | | |
| Phage- | F | P | — | W | — | D | W | E | W | D | N | N | — | H | 0.09 | 0.22 | 0.09 | 2375 |
| 614 | | | | | | | | | | | | | | | | | | |
| Phage- | — | D | — | — | — | D | G | — | — | L | A | A | — | H | 0.10 | 0.22 | 0.11 | 2376 |
| 615 | | | | | | | | | | | | | | | | | | |
| Phage- | — | D | — | W | — | D | W | E | — | Y | Y | S | — | D | 0.08 | 0.22 | 0.07 | 2377 |
| 616 | | | | | | | | | | | | | | | | | | |
| Phage- | — | — | — | — | — | — | Y | — | — | — | Y | D | — | T | 0.07 | 0.21 | 0.10 | 2378 |
| 617 | | | | | | | | | | | | | | | | | | |
| Phage- | N | L | — | — | — | — | W | E | N | F | A | D | — | F | 0.08 | 0.21 | 0.08 | 2379 |
| 618 | | | | | | | | | | | | | | | | | | |
| Phage- | Y | L | — | — | — | — | L | E | V | F | F | V | — | D | 0.12 | 0.21 | 0.10 | 2380 |
| 619 | | | | | | | | | | | | | | | | | | |
| Phage- | I | F | — | — | — | — | L | E | D | Y | S | I | — | F | 0.09 | 0.21 | 0.08 | 2381 |
| 620 | | | | | | | | | | | | | | | | | | |
| Phage- | D | — | — | — | — | — | L | E | Q | Y | D | L | — | F | 0.09 | 0.21 | 0.08 | 2382 |
| 621 | | | | | | | | | | | | | | | | | | |
| Phage- | L | L | — | — | — | V | N | E | D | P | L | D | — | Y | 0.11 | 0.21 | 0.13 | 2383 |
| 622 | | | | | | | | | | | | | | | | | | |
| Phage- | I | D | — | — | — | — | — | — | — | — | — | F | — | Y | 0.08 | 0.21 | 0.08 | 2384 |
| 623 | | | | | | | | | | | | | | | | | | |
| Phage- | I | I | — | — | — | — | V | E | — | I | D | I | — | S | 0.08 | 0.21 | 0.08 | 2385 |
| 624 | | | | | | | | | | | | | | | | | | |
| Phage- | I | A | — | W | — | D | W | E | D | Y | S | S | — | P | 0.08 | 0.21 | 0.11 | 2386 |
| 625 | | | | | | | | | | | | | | | | | | |
| Phage- | Y | — | — | — | — | — | V | E | D | I | N | D | — | I | 0.09 | 0.21 | 0.07 | 2387 |
| 626 | | | | | | | | | | | | | | | | | | |
| Phage- | N | I | — | — | — | — | M | — | — | — | I | D | — | I | 0.08 | 0.21 | 0.07 | 2388 |
| 627 | | | | | | | | | | | | | | | | | | |
| Phage- | F | D | — | W | — | D | W | E | — | L | — | S | — | Y | 0.07 | 0.21 | 0.08 | 2389 |
| 628 | | | | | | | | | | | | | | | | | | |
| Phage- | Y | F | I | — | — | — | W | E | D | H | F | F | — | D | 0.09 | 0.21 | 0.19 | 2390 |
| 629 | | | | | | | | | | | | | | | | | | |
| Phage- | T | — | — | — | — | — | — | E | — | D | S | Y | — | D | 0.12 | 0.20 | 0.09 | 2391 |
| 630 | | | | | | | | | | | | | | | | | | |
| Phage- | N | L | — | — | — | — | V | E | L | I | D | I | — | S | 0.11 | 0.20 | 0.09 | 2392 |
| 631 | | | | | | | | | | | | | | | | | | |
| Phage- | D | N | — | — | — | — | W | E | — | V | Y | L | — | N | 0.08 | 0.20 | 0.08 | 2393 |
| 632 | | | | | | | | | | | | | | | | | | |
| Phage- | F | L | — | — | — | — | — | — | — | — | D | L | — | F | 0.08 | 0.20 | 0.09 | 2394 |
| 633 | | | | | | | | | | | | | | | | | | |
| Phage- | H | I | — | — | — | — | Q | — | — | — | I | — | — | T | 0.09 | 0.20 | 0.19 | 2395 |
| 634 | | | | | | | | | | | | | | | | | | |
| Phage- | F | D | — | W | — | D | W | E | D | N | S | Y | — | D | 0.10 | 0.20 | 0.09 | 2396 |
| 635 | | | | | | | | | | | | | | | | | | |
| Phage- | T | A | — | — | — | — | W | E | F | D | F | N | — | D | 0.08 | 0.20 | 0.07 | 2397 |
| 636 | | | | | | | | | | | | | | | | | | |
| Phage- | H | H | — | W | — | D | W | E | D | Y | S | T | — | P | 0.10 | 0.20 | 0.11 | 2398 |
| 637 | | | | | | | | | | | | | | | | | | |
| Phage- | Y | — | — | — | — | — | — | — | — | — | — | N | — | F | 0.07 | 0.20 | 0.08 | 2399 |
| 638 | | | | | | | | | | | | | | | | | | |
| Phage- | L | H | — | W | — | D | W | E | — | I | D | I | — | D | 0.08 | 0.20 | 0.09 | 2400 |
| 639 | | | | | | | | | | | | | | | | | | |
| Phage- | D | I | — | — | — | D | G | Q | — | D | F | V | — | S | 0.08 | 0.20 | 0.09 | 2401 |
| 640 | | | | | | | | | | | | | | | | | | |
| Phage- | D | V | — | W | — | D | W | E | V | N | Y | F | — | D | 0.09 | 0.20 | 0.07 | 2402 |
| 641 | | | | | | | | | | | | | | | | | | |
| Phage- | — | N | — | — | — | — | M | — | — | — | I | D | — | A | 0.12 | 0.20 | 0.10 | 2403 |
| 642 | | | | | | | | | | | | | | | | | | |
| Phage- | D | N | — | — | — | — | — | — | — | A | T | V | — | N | 0.11 | 0.19 | 0.19 | 2404 |
| 643 | | | | | | | | | | | | | | | | | | |
| Phage- | D | L | — | — | — | — | — | E | — | V | H | N | — | N | 0.08 | 0.19 | 0.08 | 2405 |
| 644 | | | | | | | | | | | | | | | | | | |
| Phage- | — | N | — | — | — | — | — | — | — | — | S | Y | — | F | 0.13 | 0.19 | 0.09 | 2406 |
| 645 | | | | | | | | | | | | | | | | | | |
| Phage- | N | I | — | W | — | D | W | E | — | D | N | F | — | S | 0.08 | 0.19 | 0.08 | 2407 |
| 646 | | | | | | | | | | | | | | | | | | |
| Phage- | F | V | — | — | — | — | W | E | V | Y | D | D | — | D | 0.08 | 0.19 | 0.08 | 2408 |
| 647 | | | | | | | | | | | | | | | | | | |
| Phage- | A | — | — | — | — | — | L | E | V | V | H | L | — | V | 0.10 | 0.19 | 0.17 | 2409 |
| 648 | | | | | | | | | | | | | | | | | | |
| Phage- | P | F | — | — | — | — | M | — | — | T | I | D | — | Y | 0.07 | 0.19 | 0.09 | 2410 |
| 649 | | | | | | | | | | | | | | | | | | |
| Phage- | L | L | — | — | — | V | M | E | D | V | F | A | — | Y | 0.08 | 0.19 | 0.08 | 2411 |
| 650 | | | | | | | | | | | | | | | | | | |
| Phage- | D | L | — | — | — | — | — | — | — | — | T | N | — | Y | 0.07 | 0.19 | 0.08 | 2412 |
| 651 | | | | | | | | | | | | | | | | | | |
| Phage- | H | D | — | — | — | — | M | E | — | Y | Y | L | — | P | 0.10 | 0.18 | 0.10 | 2413 |
| 652 | | | | | | | | | | | | | | | | | | |
| Phage- | T | D | — | — | — | — | Y | — | — | — | I | I | — | P | 0.08 | 0.18 | 0.09 | 2414 |
| 653 | | | | | | | | | | | | | | | | | | |
| Phage- | — | L | — | W | — | D | W | E | D | Y | A | D | — | N | 0.09 | 0.18 | 0.08 | 2415 |
| 654 | | | | | | | | | | | | | | | | | | |
| Phage- | N | D | — | — | — | — | L | — | — | — | L | T | — | D | 0.07 | 0.18 | 0.09 | 2416 |
| 655 | | | | | | | | | | | | | | | | | | |
| Phage- | I | — | — | — | — | — | L | — | — | — | I | A | — | Y | 0.11 | 0.18 | 0.08 | 2417 |
| 656 | | | | | | | | | | | | | | | | | | |
| Phage- | N | — | — | — | — | — | V | E | — | F | N | F | — | H | 0.11 | 0.18 | 0.14 | 2418 |
| 657 | | | | | | | | | | | | | | | | | | |
| Phage- | D | V | — | — | — | — | I | E | — | Y | S | F | — | I | 0.08 | 0.18 | 0.09 | 2419 |
| 658 | | | | | | | | | | | | | | | | | | |
| Phage- | D | L | — | — | — | — | V | E | — | I | T | D | — | A | 0.10 | 0.18 | 0.12 | 2420 |
| 659 | | | | | | | | | | | | | | | | | | |
| Phage- | H | D | — | — | — | — | — | — | — | — | — | F | — | I | 0.12 | 0.18 | 0.12 | 2421 |
| 660 | | | | | | | | | | | | | | | | | | |
| Phage- | P | L | — | — | — | V | L | E | — | D | I | Y | — | Y | 0.10 | 0.18 | 0.13 | 2422 |
| 661 | | | | | | | | | | | | | | | | | | |
| Phage- | D | L | — | — | — | — | — | E | D | I | I | D | — | N | 0.10 | 0.18 | 0.11 | 2423 |
| 662 | | | | | | | | | | | | | | | | | | |
| Phage- | D | — | — | — | — | — | V | E | V | P | S | N | — | N | 0.10 | 0.18 | 0.18 | 2424 |
| 663 | | | | | | | | | | | | | | | | | | |
| Phage- | I | I | — | — | — | — | L | — | — | — | T | A | — | D | 0.10 | 0.18 | 0.09 | 2425 |
| 664 | | | | | | | | | | | | | | | | | | |
| Phage- | D | H | — | — | — | — | — | — | — | — | — | N | — | D | 0.10 | 0.18 | 0.14 | 2426 |
| 665 | | | | | | | | | | | | | | | | | | |
| Phage- | F | D | — | — | — | — | — | — | — | — | L | Y | — | S | 0.07 | 0.18 | 0.07 | 2427 |
| 666 | | | | | | | | | | | | | | | | | | |
| Phage- | F | A | — | W | — | D | W | E | — | V | Y | I | — | Y | 0.08 | 0.18 | 0.08 | 2428 |
| 667 | | | | | | | | | | | | | | | | | | |
| Phage- | L | — | — | — | — | — | — | — | — | — | L | D | — | S | 0.08 | 0.18 | 0.09 | 2429 |
| 668 | | | | | | | | | | | | | | | | | | |
| Phage- | D | L | — | — | — | — | L | E | — | A | F | L | — | A | 0.09 | 0.18 | 0.08 | 2430 |
| 669 | | | | | | | | | | | | | | | | | | |
| Phage- | F | A | — | — | — | — | L | — | — | T | L | T | — | L | 0.10 | 0.18 | 0.08 | 2431 |
| 670 | | | | | | | | | | | | | | | | | | |
| Phage- | F | D | — | — | — | — | V | E | — | I | S | N | — | D | 0.17 | 0.18 | 0.10 | 2432 |
| 671 | | | | | | | | | | | | | | | | | | |
| Phage- | — | H | — | — | — | — | L | E | Y | P | F | D | — | N | 0.09 | 0.17 | 0.16 | 2433 |
| 672 | | | | | | | | | | | | | | | | | | |
| Phage- | A | — | — | — | — | — | — | E | — | H | T | T | — | N | 0.10 | 0.17 | 0.15 | 2434 |
| 673 | | | | | | | | | | | | | | | | | | |
| Phage- | L | — | — | — | — | V | S | E | Q | F | T | F | — | I | 0.08 | 0.17 | 0.08 | 2435 |
| 674 | | | | | | | | | | | | | | | | | | |
| Phage- | D | — | — | — | — | — | L | — | — | — | Y | D | — | N | 0.10 | 0.17 | 0.09 | 2436 |
| 675 | | | | | | | | | | | | | | | | | | |
| Phage- | F | — | — | — | — | — | W | E | — | F | D | V | — | I | 0.13 | 0.17 | 0.15 | 2437 |
| 676 | | | | | | | | | | | | | | | | | | |
| Phage- | F | T | — | — | — | — | V | E | — | Y | D | H | — | I | 0.08 | 0.17 | 0.09 | 2438 |
| 677 | | | | | | | | | | | | | | | | | | |
| Phage- | Y | N | — | — | — | — | — | — | — | — | — | T | — | F | 0.12 | 0.17 | 0.11 | 2439 |
| 678 | | | | | | | | | | | | | | | | | | |
| Phage- | A | V | — | — | — | — | N | — | — | — | N | S | — | A | 0.08 | 0.17 | 0.08 | 2440 |
| 679 | | | | | | | | | | | | | | | | | | |
| Phage- | I | — | — | W | — | D | W | E | V | P | N | D | — | A | 0.10 | 0.17 | 0.09 | 2441 |
| 680 | | | | | | | | | | | | | | | | | | |
| Phage- | Y | F | — | — | — | — | — | E | — | F | F | H | — | Y | 0.12 | 0.17 | 0.12 | 2442 |
| 681 | | | | | | | | | | | | | | | | | | |
| Phage- | Y | V | — | — | — | D | G | — | — | — | S | F | — | D | 0.12 | 0.17 | 0.12 | 2443 |
| 682 | | | | | | | | | | | | | | | | | | |
| Phage- | I | S | — | — | — | — | V | E | — | F | F | Y | — | Y | 0.10 | 0.17 | 0.08 | 2444 |
| 683 | | | | | | | | | | | | | | | | | | |
| Phage- | L | I | — | — | — | V | — | E | — | D | — | Y | — | D | 0.17 | 0.17 | 0.15 | 2445 |
| 684 | | | | | | | | | | | | | | | | | | |
| Phage- | — | — | — | — | — | — | V | E | D | H | N | Y | — | A | 0.14 | 0.17 | 0.16 | 2446 |
| 685 | | | | | | | | | | | | | | | | | | |
| Phage- | L | D | — | — | — | — | — | E | F | V | Y | I | — | A | 0.08 | 0.17 | 0.10 | 2447 |
| 686 | | | | | | | | | | | | | | | | | | |
| Phage- | Y | D | — | — | — | — | — | E | — | D | L | P | — | I | 0.17 | 0.17 | 0.11 | 2448 |
| 687 | | | | | | | | | | | | | | | | | | |
| Phage- | D | V | — | — | — | — | V | E | — | D | Y | Y | — | D | 0.10 | 0.17 | 0.14 | 2449 |
| 688 | | | | | | | | | | | | | | | | | | |
| Phage- | N | D | — | W | — | D | W | E | Y | D | N | V | — | V | 0.08 | 0.17 | 0.10 | 2450 |
| 689 | | | | | | | | | | | | | | | | | | |
| Phage- | D | L | — | — | — | — | — | E | V | A | N | D | — | N | 0.10 | 0.16 | 0.16 | 2451 |
| 690 | | | | | | | | | | | | | | | | | | |
| Phage- | H | D | — | — | — | — | L | — | — | — | I | S | — | N | 0.09 | 0.16 | 0.07 | 2452 |
| 691 | | | | | | | | | | | | | | | | | | |
| Phage- | L | D | — | W | — | D | W | E | — | T | T | H | — | D | 0.08 | 0.16 | 0.08 | 2453 |
| 692 | | | | | | | | | | | | | | | | | | |
| Phage- | I | I | — | — | — | — | V | E | — | D | D | Y | — | L | 0.09 | 0.16 | 0.09 | 2454 |
| 693 | | | | | | | | | | | | | | | | | | |
| Phage- | Y | — | — | W | — | D | W | E | — | V | I | I | — | D | 0.09 | 0.16 | 0.09 | 2455 |
| 694 | | | | | | | | | | | | | | | | | | |
| Phage- | F | D | — | — | — | — | I | — | — | Y | T | N | — | N | 0.12 | 0.16 | 0.09 | 2456 |
| 695 | | | | | | | | | | | | | | | | | | |
| Phage- | I | T | — | — | — | — | L | — | — | T | I | N | — | D | 0.08 | 0.16 | 0.11 | 2457 |
| 696 | | | | | | | | | | | | | | | | | | |
| Phage- | D | S | — | — | — | — | V | E | — | D | I | Y | — | I | 0.07 | 0.16 | 0.08 | 2458 |
| 697 | | | | | | | | | | | | | | | | | | |
| Phage- | Y | L | — | — | — | — | — | — | — | — | G | N | — | H | 0.07 | 0.16 | 0.08 | 2459 |
| 698 | | | | | | | | | | | | | | | | | | |
| Phage- | D | N | — | — | — | — | L | P | — | D | Y | F | — | D | 0.08 | 0.16 | 0.10 | 2460 |
| 699 | | | | | | | | | | | | | | | | | | |
| Phage- | — | L | — | — | — | — | — | E | — | V | S | N | — | N | 0.11 | 0.16 | 0.08 | 2461 |
| 700 | | | | | | | | | | | | | | | | | | |
| Phage- | — | D | — | W | — | D | W | E | — | D | I | V | — | D | 0.10 | 0.16 | 0.09 | 2462 |
| 701 | | | | | | | | | | | | | | | | | | |
| Phage- | — | — | — | W | — | D | W | E | D | N | F | P | — | Y | 0.07 | 0.16 | 0.07 | 2463 |
| 702 | | | | | | | | | | | | | | | | | | |
| Phage- | D | — | — | — | — | — | V | E | — | H | F | N | — | H | 0.08 | 0.16 | 0.08 | 2464 |
| 703 | | | | | | | | | | | | | | | | | | |
| Phage- | A | D | — | — | — | — | I | E | — | D | A | Y | — | Y | 0.12 | 0.16 | 0.09 | 2465 |
| 704 | | | | | | | | | | | | | | | | | | |
| Phage- | I | L | — | W | — | D | W | E | D | A | T | F | — | Y | 0.09 | 0.16 | 0.07 | 2466 |
| 705 | | | | | | | | | | | | | | | | | | |
| Phage- | I | H | — | W | — | D | W | E | D | F | N | I | — | P | 0.09 | 0.16 | 0.08 | 2467 |
| 706 | | | | | | | | | | | | | | | | | | |
| Phage- | T | I | — | — | — | — | V | E | D | Y | N | D | — | I | 0.07 | 0.16 | 0.07 | 2468 |
| 707 | | | | | | | | | | | | | | | | | | |
| Phage- | D | D | — | — | — | — | L | — | — | — | — | A | — | I | 0.08 | 0.16 | 0.08 | 2469 |
| 708 | | | | | | | | | | | | | | | | | | |
| Phage- | D | D | — | W | — | D | W | E | D | H | I | F | — | F | 0.13 | 0.16 | 0.08 | 2470 |
| 709 | | | | | | | | | | | | | | | | | | |
| Phage- | — | N | — | — | — | — | V | E | — | I | I | F | — | D | 0.12 | 0.15 | 0.12 | 2471 |
| 710 | | | | | | | | | | | | | | | | | | |
| Phage- | I | F | — | W | — | D | W | E | D | D | T | V | — | I | 0.08 | 0.15 | 0.09 | 2472 |
| 711 | | | | | | | | | | | | | | | | | | |
| Phage- | I | I | — | — | — | — | — | E | — | I | S | D | — | L | 0.12 | 0.15 | 0.15 | 2473 |
| 712 | | | | | | | | | | | | | | | | | | |
| Phage- | F | D | — | — | — | — | V | E | — | Y | N | D | — | D | 0.11 | 0.15 | 0.09 | 2474 |
| 713 | | | | | | | | | | | | | | | | | | |
| Phage- | N | D | — | — | — | — | L | — | — | T | L | Y | — | I | 0.08 | 0.15 | 0.10 | 2475 |
| 714 | | | | | | | | | | | | | | | | | | |
| Phage- | A | I | — | — | — | — | L | E | — | D | I | S | — | N | 0.12 | 0.15 | 0.17 | 2476 |
| 715 | | | | | | | | | | | | | | | | | | |
| Phage- | H | L | — | — | — | — | — | — | — | — | T | N | — | Y | 0.07 | 0.15 | 0.07 | 2477 |
| 716 | | | | | | | | | | | | | | | | | | |
| Phage- | S | — | — | — | — | — | L | — | — | — | — | A | — | I | 0.10 | 0.15 | 0.11 | 2478 |
| 717 | | | | | | | | | | | | | | | | | | |
| Phage- | L | — | — | — | — | — | — | E | — | L | A | D | — | T | 0.08 | 0.15 | 0.08 | 2479 |
| 718 | | | | | | | | | | | | | | | | | | |
| Phage- | I | D | — | — | — | — | L | — | — | — | I | A | — | N | 0.07 | 0.15 | 0.08 | 2480 |
| 719 | | | | | | | | | | | | | | | | | | |
| Phage- | F | D | — | — | — | D | G | Q | — | D | L | V | — | N | 0.10 | 0.15 | 0.08 | 2481 |
| 720 | | | | | | | | | | | | | | | | | | |
| Phage- | N | L | — | — | — | — | — | E | — | F | F | D | — | Y | 0.09 | 0.15 | 0.15 | 2482 |
| 721 | | | | | | | | | | | | | | | | | | |
| Phage- | S | I | — | — | — | — | L | Q | — | D | I | V | — | P | 0.09 | 0.14 | 0.14 | 2483 |
| 722 | | | | | | | | | | | | | | | | | | |
| Phage- | N | P | — | — | — | — | Y | — | — | — | A | H | — | D | 0.08 | 0.14 | 0.08 | 2484 |
| 723 | | | | | | | | | | | | | | | | | | |
| Phage- | Y | D | — | — | — | — | L | — | — | Y | Y | N | — | N | 0.12 | 0.14 | 0.11 | 2485 |
| 724 | | | | | | | | | | | | | | | | | | |
| Phage- | — | D | — | — | — | — | L | — | — | T | I | F | — | D | 0.07 | 0.14 | 0.08 | 2486 |
| 725 | | | | | | | | | | | | | | | | | | |
| Phage- | D | N | — | — | — | — | L | — | — | — | — | T | — | T | 0.09 | 0.14 | 0.10 | 2487 |
| 726 | | | | | | | | | | | | | | | | | | |
| Phage- | — | D | — | — | — | — | L | — | — | — | S | Y | — | D | 0.10 | 0.14 | 0.13 | 2488 |
| 727 | | | | | | | | | | | | | | | | | | |
| Phage- | Y | — | — | — | — | — | — | E | F | I | D | F | — | F | 0.07 | 0.14 | 0.07 | 2489 |
| 728 | | | | | | | | | | | | | | | | | | |
| Phage- | Y | D | — | W | — | D | W | E | V | I | T | Y | — | N | 0.08 | 0.14 | 0.09 | 2490 |
| 729 | | | | | | | | | | | | | | | | | | |
| Phage- | F | D | — | — | — | — | I | E | — | D | F | F | — | V | 0.06 | 0.14 | 0.07 | 2491 |
| 730 | | | | | | | | | | | | | | | | | | |
| Phage- | I | F | — | W | — | D | W | — | D | I | N | F | — | D | 0.10 | 0.14 | 0.14 | 2492 |
| 731 | | | | | | | | | | | | | | | | | | |
| Phage- | N | F | — | — | — | — | L | P | — | D | I | T | — | Y | 0.37 | 0.14 | 0.09 | 2493 |
| 732 | | | | | | | | | | | | | | | | | | |
| Phage- | S | L | — | — | — | — | — | E | — | Y | Y | H | — | L | 0.09 | 0.14 | 0.07 | 2494 |
| 733 | | | | | | | | | | | | | | | | | | |
| Phage- | A | S | — | — | — | — | L | — | — | — | L | D | — | L | 0.12 | 0.14 | 0.13 | 2495 |
| 734 | | | | | | | | | | | | | | | | | | |
| Phage- | S | F | — | — | — | — | R | E | W | D | L | A | — | Y | 0.09 | 0.14 | 0.08 | 2496 |
| 735 | | | | | | | | | | | | | | | | | | |
| Phage- | H | L | — | — | — | — | — | E | D | V | L | D | — | I | 0.08 | 0.14 | 0.12 | 2497 |
| 736 | | | | | | | | | | | | | | | | | | |
| Phage- | L | D | — | — | — | D | G | — | — | F | Y | Y | — | L | 0.20 | 0.14 | 0.09 | 2498 |
| 737 | | | | | | | | | | | | | | | | | | |
| Phage- | D | N | — | W | — | D | W | E | — | D | I | A | — | T | 0.16 | 0.14 | 0.11 | 2499 |
| 738 | | | | | | | | | | | | | | | | | | |
| Phage- | S | D | — | — | — | — | L | — | — | T | I | H | — | I | 0.09 | 0.14 | 0.08 | 2500 |
| 739 | | | | | | | | | | | | | | | | | | |
| Phage- | N | S | — | — | — | D | G | — | — | — | — | D | — | L | 0.08 | 0.14 | 0.08 | 2501 |
| 740 | | | | | | | | | | | | | | | | | | |
| Phage- | D | L | — | — | — | — | L | — | — | — | T | L | — | I | 0.07 | 0.14 | 0.08 | 2502 |
| 741 | | | | | | | | | | | | | | | | | | |
| Phage- | F | D | — | — | — | — | S | — | — | — | F | N | — | Y | 0.09 | 0.14 | 0.11 | 2503 |
| 742 | | | | | | | | | | | | | | | | | | |
| Phage- | D | L | — | — | — | — | — | E | — | D | D | I | — | Y | 0.12 | 0.14 | 0.13 | 2504 |
| 743 | | | | | | | | | | | | | | | | | | |
| Phage- | H | A | — | — | — | — | — | E | — | D | T | Y | — | F | 0.10 | 0.14 | 0.14 | 2505 |
| 744 | | | | | | | | | | | | | | | | | | |
| Phage- | S | D | — | — | — | — | L | — | — | — | — | A | — | T | 0.11 | 0.14 | 0.08 | 2506 |
| 745 | | | | | | | | | | | | | | | | | | |
| Phage- | I | V | — | — | — | — | L | P | — | D | Y | N | — | Y | 0.09 | 0.13 | 0.11 | 2507 |
| 746 | | | | | | | | | | | | | | | | | | |
| Phage- | D | L | — | — | — | — | — | — | — | I | F | I | — | F | 0.09 | 0.13 | 0.08 | 2508 |
| 747 | | | | | | | | | | | | | | | | | | |
| Phage- | Y | D | — | — | — | — | — | — | — | T | L | T | — | N | 0.13 | 0.13 | 0.08 | 2509 |
| 748 | | | | | | | | | | | | | | | | | | |
| Phage- | Y | D | — | — | — | — | V | E | — | T | — | N | — | D | 0.11 | 0.13 | 0.12 | 2510 |
| 749 | | | | | | | | | | | | | | | | | | |
| Phage- | — | F | — | — | — | — | I | E | D | D | H | V | — | I | 0.07 | 0.13 | 0.07 | 2511 |
| 750 | | | | | | | | | | | | | | | | | | |
| Phage- | F | I | — | — | — | — | W | E | D | D | Y | A | — | S | 0.12 | 0.13 | 0.12 | 2512 |
| 751 | | | | | | | | | | | | | | | | | | |
| Phage- | N | F | — | — | — | — | — | — | — | — | — | — | — | N | 0.10 | 0.13 | 0.09 | 2513 |
| 752 | | | | | | | | | | | | | | | | | | |
| Phage- | N | L | — | — | — | — | V | E | — | I | L | I | — | D | 0.07 | 0.13 | 0.07 | 2514 |
| 753 | | | | | | | | | | | | | | | | | | |
| Phage- | D | S | — | — | — | — | V | E | — | Y | D | L | — | N | 0.11 | 0.13 | 0.08 | 2515 |
| 754 | | | | | | | | | | | | | | | | | | |
| Phage- | T | L | — | — | — | — | — | E | — | T | T | D | — | N | 0.09 | 0.13 | 0.08 | 2516 |
| 755 | | | | | | | | | | | | | | | | | | |
| Phage- | T | V | — | — | — | K | M | E | M | N | S | T | — | D | 0.09 | 0.13 | 0.09 | 2517 |
| 756 | | | | | | | | | | | | | | | | | | |
| Phage- | — | H | — | W | — | D | W | E | D | A | — | S | — | N | 0.10 | 0.12 | 0.11 | 2518 |
| 757 | | | | | | | | | | | | | | | | | | |
| Phage- | D | L | — | — | — | D | G | N | — | L | D | F | — | F | 0.08 | 0.12 | 0.08 | 2519 |
| 758 | | | | | | | | | | | | | | | | | | |
| Phage- | D | L | — | — | — | D | G | E | — | H | Y | Y | — | D | 0.09 | 0.12 | 0.07 | 2520 |
| 759 | | | | | | | | | | | | | | | | | | |
| Phage- | F | N | — | — | — | — | V | E | — | I | L | L | — | T | 0.11 | 0.12 | 0.12 | 2521 |
| 760 | | | | | | | | | | | | | | | | | | |
| Phage- | H | — | — | — | — | — | V | E | N | I | N | D | — | I | 0.09 | 0.12 | 0.07 | 2522 |
| 761 | | | | | | | | | | | | | | | | | | |
| Phage- | I | D | — | — | — | — | L | — | — | — | — | I | — | D | 0.09 | 0.12 | 0.08 | 2523 |
| 762 | | | | | | | | | | | | | | | | | | |
| Phage- | I | F | — | — | — | — | I | E | Q | P | A | L | — | Y | 0.08 | 0.12 | 0.09 | 2524 |
| 763 | | | | | | | | | | | | | | | | | | |
| Phage- | Y | D | — | — | — | — | — | Q | — | D | L | V | — | P | 0.10 | 0.12 | 0.08 | 2525 |
| 764 | | | | | | | | | | | | | | | | | | |
| Phage- | H | A | — | W | — | D | W | E | — | P | N | Y | — | D | 0.13 | 0.12 | 0.09 | 2526 |
| 765 | | | | | | | | | | | | | | | | | | |
| Phage- | N | V | — | W | — | D | W | E | — | D | Y | N | — | Y | 0.11 | 0.12 | 0.10 | 2527 |
| 766 | | | | | | | | | | | | | | | | | | |
| Phage- | S | F | — | — | Q | — | L | G | D | N | Y | D | — | I | 0.09 | 0.12 | 0.12 | 2528 |
| 767 | | | | | | | | | | | | | | | | | | |
| Phage- | D | D | — | — | — | — | L | — | — | T | T | V | — | Y | 0.08 | 0.12 | 0.09 | 2529 |
| 768 | | | | | | | | | | | | | | | | | | |
| Phage- | N | F | — | — | — | — | W | E | V | A | T | L | — | L | 0.09 | 0.12 | 0.14 | 2530 |
| 769 | | | | | | | | | | | | | | | | | | |
| Phage- | D | L | — | — | — | — | V | E | — | D | T | Y | — | N | 0.09 | 0.11 | 0.07 | 2531 |
| 770 | | | | | | | | | | | | | | | | | | |
| Phage- | A | L | — | — | — | — | V | E | Q | V | D | L | — | T | 0.08 | 0.11 | 0.08 | 2532 |
| 771 | | | | | | | | | | | | | | | | | | |
| Phage- | D | D | — | — | — | — | L | — | — | — | — | N | — | N | 0.08 | 0.11 | 0.08 | 2533 |
| 772 | | | | | | | | | | | | | | | | | | |
| Phage- | I | F | — | — | — | — | — | E | Q | I | I | Y | — | D | 0.09 | 0.11 | 0.11 | 2534 |
| 773 | | | | | | | | | | | | | | | | | | |
| Phage- | S | D | — | W | — | D | W | E | — | V | Y | Y | — | S | 0.09 | 0.11 | 0.10 | 2535 |
| 774 | | | | | | | | | | | | | | | | | | |
| Phage- | F | F | — | — | — | D | G | — | — | V | A | I | — | D | 0.09 | 0.11 | 0.07 | 2536 |
| 775 | | | | | | | | | | | | | | | | | | |
| Phage- | D | D | — | W | — | D | W | E | D | D | — | Y | — | Y | 0.11 | 0.11 | 0.07 | 2537 |
| 776 | | | | | | | | | | | | | | | | | | |
| Phage- | Y | D | — | — | — | — | — | — | — | T | — | V | — | P | 0.08 | 0.11 | 0.08 | 2538 |
| 777 | | | | | | | | | | | | | | | | | | |
| Phage- | S | — | — | — | — | — | L | — | — | — | — | N | — | V | 0.10 | 0.11 | 0.08 | 2539 |
| 778 | | | | | | | | | | | | | | | | | | |
| Phage- | A | F | — | V | S | F | Q | Q | S | L | P | H | — | D | 0.09 | 0.11 | 0.10 | 2540 |
| 779 | | | | | | | | | | | | | | | | | | |
| Phage- | — | N | — | — | — | — | V | E | — | Y | F | V | — | F | 0.09 | 0.11 | 0.09 | 2541 |
| 780 | | | | | | | | | | | | | | | | | | |
| Phage- | T | N | — | W | — | D | W | E | — | D | F | A | — | V | 0.07 | 0.11 | 0.08 | 2542 |
| 781 | | | | | | | | | | | | | | | | | | |
| Phage- | D | D | — | — | — | — | — | E | — | I | I | L | — | F | 0.08 | 0.10 | 0.08 | 2543 |
| 782 | | | | | | | | | | | | | | | | | | |
| Phage- | N | S | — | — | — | — | L | E | D | Y | H | L | — | P | 0.08 | 0.10 | 0.10 | 2544 |
| 783 | | | | | | | | | | | | | | | | | | |
| Phage- | I | H | — | — | — | D | S | — | G | F | D | F | — | D | 0.09 | 0.10 | 0.08 | 2545 |
| 784 | | | | | | | | | | | | | | | | | | |
| Phage- | F | D | — | — | — | — | L | E | — | D | H | L | — | F | 0.08 | 0.10 | 0.08 | 2546 |
| 785 | | | | | | | | | | | | | | | | | | |
| Phage- | T | V | — | W | — | D | — | E | — | Y | A | D | — | D | 0.10 | 0.10 | 0.08 | 2547 |
| 786 | | | | | | | | | | | | | | | | | | |
| Phage- | Y | F | — | W | — | D | W | E | — | A | A | D | — | L | 0.09 | 0.10 | 0.09 | 2548 |
| 787 | | | | | | | | | | | | | | | | | | |
| Phage- | — | S | — | — | — | — | — | — | — | — | — | D | — | I | 0.08 | 0.10 | 0.07 | 2549 |
| 788 | | | | | | | | | | | | | | | | | | |
| Phage- | A | V | — | — | — | — | L | P | — | D | I | V | — | Y | 0.08 | 0.10 | 0.08 | 2550 |
| 789 | | | | | | | | | | | | | | | | | | |
| Phage- | — | L | — | — | — | — | V | E | — | Y | H | L | — | A | 0.08 | 0.10 | 0.30 | 2551 |
| 790 | | | | | | | | | | | | | | | | | | |
| Phage- | S | L | — | W | — | D | W | E | — | V | D | N | — | F | 0.12 | 0.10 | 0.11 | 2552 |
| 791 | | | | | | | | | | | | | | | | | | |
| Phage- | T | I | — | — | — | D | G | Q | — | D | Y | N | — | H | 0.07 | 0.10 | 0.07 | 2553 |
| 792 | | | | | | | | | | | | | | | | | | |
| Phage- | F | L | — | — | — | — | G | E | P | T | Y | L | — | T | 0.12 | 0.10 | 0.09 | 2554 |
| 793 | | | | | | | | | | | | | | | | | | |
| Phage- | D | D | — | W | L | — | Q | H | D | I | Y | V | — | A | 0.10 | 0.10 | 0.08 | 2555 |
| 794 | | | | | | | | | | | | | | | | | | |
| Phage- | I | F | — | — | — | — | V | E | — | V | A | F | — | F | 0.07 | 0.10 | 0.08 | 2556 |
| 795 | | | | | | | | | | | | | | | | | | |
| Phage- | A | L | — | — | — | — | V | E | D | D | Y | D | — | L | 0.09 | 0.10 | 0.09 | 2557 |
| 796 | | | | | | | | | | | | | | | | | | |
| Phage- | D | D | — | — | — | — | I | E | L | Y | L | T | — | A | 0.08 | 0.10 | 0.08 | 2558 |
| 797 | | | | | | | | | | | | | | | | | | |
| Phage- | T | D | — | W | — | D | W | E | D | D | S | I | — | D | 0.07 | 0.10 | 0.07 | 2559 |
| 798 | | | | | | | | | | | | | | | | | | |
| Phage- | S | I | — | — | — | — | L | E | — | I | F | L | — | N | 0.08 | 0.10 | 0.08 | 2560 |
| 799 | | | | | | | | | | | | | | | | | | |
| Phage- | N | D | — | — | — | — | L | E | — | D | I | L | — | F | 0.07 | 0.10 | 0.09 | 2561 |
| 800 | | | | | | | | | | | | | | | | | | |
| Phage- | D | D | — | — | — | — | L | — | — | T | Y | S | — | Y | 0.09 | 0.09 | 0.08 | 2562 |
| 801 | | | | | | | | | | | | | | | | | | |
| Phage- | F | V | — | — | — | — | W | E | — | I | D | L | — | I | 0.10 | 0.09 | 0.09 | 2563 |
| 802 | | | | | | | | | | | | | | | | | | |
| Phage- | Y | D | — | — | — | — | L | — | — | — | L | S | — | P | 0.07 | 0.09 | 0.07 | 2564 |
| 803 | | | | | | | | | | | | | | | | | | |
| Phage- | N | L | — | — | — | — | L | — | — | — | I | I | — | P | 0.08 | 0.09 | 0.08 | 2565 |
| 804 | | | | | | | | | | | | | | | | | | |
| Phage- | I | D | — | W | — | D | W | E | — | F | N | N | — | F | 0.08 | 0.09 | 0.09 | 2566 |
| 805 | | | | | | | | | | | | | | | | | | |
| Phage- | A | — | — | — | — | — | L | E | — | H | D | Y | — | Y | 0.08 | 0.09 | 0.09 | 2567 |
| 806 | | | | | | | | | | | | | | | | | | |
| Phage- | D | D | — | S | — | Q | — | — | Q | I | D | L | — | D | 0.14 | 0.09 | 0.09 | 2568 |
| 807 | | | | | | | | | | | | | | | | | | |
| Phage- | S | — | — | — | — | — | S | — | — | — | I | Y | — | Y | 0.08 | 0.09 | 0.07 | 2569 |
| 808 | | | | | | | | | | | | | | | | | | |
| Phage- | S | L | — | — | — | — | — | Q | — | D | A | P | — | N | 0.07 | 0.09 | 0.07 | 2570 |
| 809 | | | | | | | | | | | | | | | | | | |
| Phage- | D | A | — | — | — | — | L | — | — | T | T | H | — | D | 0.09 | 0.09 | 0.08 | 2571 |
| 810 | | | | | | | | | | | | | | | | | | |
| Phage- | N | D | — | — | — | | V | E | — | V | A | D | — | F | 0.07 | 0.09 | 0.08 | 2572 |
| 811 | | | | | | | | | | | | | | | | | | |
| Phage- | Y | I | — | — | — | — | — | E | Q | D | Y | F | — | F | 0.08 | 0.09 | 0.08 | 2573 |
| 812 | | | | | | | | | | | | | | | | | | |
| Phage- | T | D | — | — | — | D | G | — | — | T | N | Y | — | F | 0.11 | 0.09 | 0.08 | 2574 |
| 813 | | | | | | | | | | | | | | | | | | |
| Phage- | Y | D | — | — | — | — | V | — | — | — | — | L | — | P | 0.08 | 0.09 | 0.08 | 2575 |
| 814 | | | | | | | | | | | | | | | | | | |
| Phage- | I | D | — | — | — | — | — | — | — | — | A | Y | — | T | 0.08 | 0.09 | 0.08 | 2576 |
| 815 | | | | | | | | | | | | | | | | | | |
| Phage- | F | D | — | — | — | — | — | E | — | F | F | H | — | Y | 0.09 | 0.09 | 0.09 | 2577 |
| 816 | | | | | | | | | | | | | | | | | | |
| Phage- | F | P | — | — | — | — | I | E | — | Y | D | Y | — | V | 0.09 | 0.09 | 0.08 | 2578 |
| 817 | | | | | | | | | | | | | | | | | | |
| Phage- | A | D | — | — | — | I | — | E | S | I | D | I | — | V | 0.09 | 0.09 | 0.07 | 2579 |
| 818 | | | | | | | | | | | | | | | | | | |
| Phage- | I | S | — | — | — | D | L | W | P | T | D | I | — | T | 0.10 | 0.09 | 0.10 | 2580 |
| 819 | | | | | | | | | | | | | | | | | | |
| Phage- | D | D | — | — | — | D | G | — | — | V | H | T | — | N | 0.09 | 0.09 | 0.07 | 2581 |
| 820 | | | | | | | | | | | | | | | | | | |
| Phage- | I | D | — | W | — | D | W | E | G | — | F | A | — | N | 0.09 | 0.09 | 0.08 | 2582 |
| 821 | | | | | | | | | | | | | | | | | | |
| Phage- | L | N | — | — | — | D | G | — | — | T | F | Y | — | D | 0.08 | 0.08 | 0.07 | 2583 |
| 822 | | | | | | | | | | | | | | | | | | |
| Phage- | I | H | — | — | — | — | L | G | A | Y | I | S | — | S | 0.08 | 0.08 | 0.09 | 2584 |
| 823 | | | | | | | | | | | | | | | | | | |
| Phage- | T | I | — | W | — | D | W | E | — | D | Y | F | — | Y | 0.08 | 0.08 | 0.08 | 2585 |
| 824 | | | | | | | | | | | | | | | | | | |
| Phage- | H | S | — | L | A | Q | — | — | Q | D | L | V | — | I | 0.07 | 0.08 | 0.07 | 2586 |
| 825 | | | | | | | | | | | | | | | | | | |
| Phage- | N | N | — | A | S | D | L | S | — | D | N | S | — | I | 0.07 | 0.08 | 0.08 | 2587 |
| 826 | | | | | | | | | | | | | | | | | | |
| Phage- | — | N | — | W | — | D | W | E | — | D | — | A | — | N | 0.09 | 0.08 | 0.07 | 2588 |
| 827 | | | | | | | | | | | | | | | | | | |
| Phage- | — | — | — | — | — | — | L | — | — | — | F | Y | — | F | 0.08 | 0.08 | 0.07 | 2589 |
| 828 | | | | | | | | | | | | | | | | | | |
| Phage- | L | F | — | — | T | V | L | — | — | F | F | D | — | D | 0.07 | 0.08 | 0.07 | 2590 |
| 829 | | | | | | | | | | | | | | | | | | |
| Phage- | F | D | — | — | — | — | L | — | — | — | — | S | — | A | 0.08 | 0.08 | 0.07 | 2591 |
| 830 | | | | | | | | | | | | | | | | | | |
| Phage- | Y | — | — | — | — | — | V | E | — | N | — | Y | — | T | 0.07 | 0.08 | 0.08 | 2592 |
| 831 | | | | | | | | | | | | | | | | | | |
| Phage- | D | — | — | — | — | — | L | — | D | — | T | I | — | H | 0.12 | 0.08 | 0.10 | 2593 |
| 832 | | | | | | | | | | | | | | | | | | |
| Phage- | — | F | — | — | — | Q | W | A | — | A | N | A | — | F | 0.09 | 0.08 | 0.07 | 2594 |
| 833 | | | | | | | | | | | | | | | | | | |
| Phage- | F | T | — | — | — | — | Y | — | — | — | I | T | — | P | 0.09 | 0.08 | 0.09 | 2595 |
| 834 | | | | | | | | | | | | | | | | | | |
| Phage- | L | N | — | — | — | V | N | — | — | — | S | V | — | T | 0.07 | 0.08 | 0.08 | 2596 |
| 835 | | | | | | | | | | | | | | | | | | |
| Phage- | A | I | — | W | — | D | W | E | — | F | S | D | — | H | 0.07 | 0.08 | 0.07 | 2597 |
| 836 | | | | | | | | | | | | | | | | | | |
| Phage- | Y | — | — | — | V | D | L | G | A | N | — | Y | — | Y | 0.10 | 0.08 | 0.09 | 2598 |
| 837 | | | | | | | | | | | | | | | | | | |
| Phage- | H | — | — | — | — | — | V | E | — | D | Y | H | — | D | 0.07 | 0.08 | 0.07 | 2599 |
| 838 | | | | | | | | | | | | | | | | | | |
| Phage- | N | D | — | — | S | L | Q | Y | D | I | P | T | — | V | 0.08 | 0.08 | 0.07 | 2600 |
| 839 | | | | | | | | | | | | | | | | | | |
| Phage- | Y | V | — | R | — | Q | L | — | V | Y | H | Y | — | N | 0.15 | 0.08 | 0.07 | 2601 |
| 840 | | | | | | | | | | | | | | | | | | |
| Phage- | H | D | — | — | — | D | G | — | — | — | I | I | — | S | 0.08 | 0.08 | 0.07 | 2602 |
| 841 | | | | | | | | | | | | | | | | | | |
| Phage- | F | D | — | — | — | — | L | — | — | T | I | I | — | P | 0.08 | 0.08 | 0.08 | 2603 |
| 842 | | | | | | | | | | | | | | | | | | |
| Phage- | — | D | — | — | S | D | R | G | — | N | A | A | — | H | 0.07 | 0.08 | 0.07 | 2604 |
| 843 | | | | | | | | | | | | | | | | | | |
| Phage- | N | I | — | L | A | Q | — | N | — | D | P | T | — | N | 0.07 | 0.08 | 0.08 | 2605 |
| 844 | | | | | | | | | | | | | | | | | | |
| Phage- | T | N | — | — | S | K | S | Q | V | — | D | H | — | I | 0.10 | 0.08 | 0.09 | 2606 |
| 845 | | | | | | | | | | | | | | | | | | |
| Phage- | N | H | — | H | — | Q | — | W | — | L | T | N | — | N | 0.09 | 0.07 | 0.07 | 2607 |
| 846 | | | | | | | | | | | | | | | | | | |
| Phage- | L | L | — | H | — | Q | G | — | L | Y | H | L | — | H | 0.09 | 0.07 | 0.08 | 2608 |
| 847 | | | | | | | | | | | | | | | | | | |
| Phage- | T | N | — | D | S | K | L | E | G | D | D | N | — | F | 0.09 | 0.07 | 0.07 | 2609 |
| 848 | | | | | | | | | | | | | | | | | | |
| Phage- | N | D | — | — | — | — | M | — | — | — | L | L | — | D | 0.08 | 0.07 | 0.07 | 2610 |
| 849 | | | | | | | | | | | | | | | | | | |
| Phage- | F | H | — | — | — | — | V | — | — | — | I | N | — | N | 0.07 | 0.07 | 0.07 | 2611 |
| 850 | | | | | | | | | | | | | | | | | | |
| Phage- | D | F | — | — | — | D | G | — | — | T | Y | V | — | S | 0.07 | 0.07 | 0.08 | 2612 |
| 851 | | | | | | | | | | | | | | | | | | |
| Phage- | — | S | — | — | — | Q | — | — | — | N | N | T | — | N | 0.07 | 0.07 | 0.08 | 2613 |
| 852 | | | | | | | | | | | | | | | | | | |
| Phage- | — | — | — | H | L | — | S | E | Q | F | D | I | — | I | 0.08 | 0.07 | 0.08 | 2614 |
| 853 | | | | | | | | | | | | | | | | | | |
| Phage- | D | D | — | — | — | — | W | E | F | V | F | F | — | D | 0.08 | 0.07 | 0.08 | 2615 |
| 854 | | | | | | | | | | | | | | | | | | |
| Phage- | Y | N | — | E | Q | Q | Q | — | — | D | P | S | — | I | 0.07 | 0.07 | 0.07 | 2616 |
| 855 | | | | | | | | | | | | | | | | | | |
| Phage- | N | T | — | — | T | — | Q | H | — | F | N | — | — | L | 0.08 | 0.07 | 0.08 | 2617 |
| 856 | | | | | | | | | | | | | | | | | | |
| Phage- | H | P | — | Q | — | G | — | E | — | V | D | Y | — | V | 0.08 | 0.07 | 0.08 | 2618 |
| 857 | | | | | | | | | | | | | | | | | | |
| Phage- | — | A | — | S | R | Q | L | G | — | D | A | Y | — | N | 0.07 | 0.07 | 0.07 | 2619 |
| 858 | | | | | | | | | | | | | | | | | | |
| Phage- | D | I | — | — | A | Q | E | V | H | V | Y | T | — | P | 0.07 | 0.07 | 0.07 | 2620 |
| 859 | | | | | | | | | | | | | | | | | | |
| Phage- | F | F | — | E | G | N | L | — | A | Y | L | L | — | L | 0.08 | 0.07 | 0.08 | 2621 |
| 860 | | | | | | | | | | | | | | | | | | |
| Phage- | Y | — | — | — | — | D | G | E | — | N | I | V | — | D | 0.07 | 0.07 | 0.07 | 2622 |
| 861 | | | | | | | | | | | | | | | | | | |

(—) indicates same amino acid as in CD3 scFv Peptide-B corresponding position (e.g. Phage-1 position).

TABLE 36
Sequences of those peptides selected for synthesis
(CD3 scFv Peptide-B Optimization)

| | | SEQ ID |
|---|---|---|
| Peptide-ID | Sequence | NO: |
| Peptide-AA | DDCWPDWEFDFACA | 229 |
| Peptide-AB | YICGLDFPDFLYCD | 230 |
| Peptide-AC | FDCWPDWEEYFVCD | 231 |
| Peptide-AD | YICWPDWEEYFDCD | 232 |
| Peptide-AE | NICWPDWEDDYFCF | 233 |
| Peptide-AF | NFCWPDWEYIYPCI | 234 |
| Peptide-AG | VDCWPDWEEDFLCI | 235 |
| Peptide-AH | HACWPDWEEYFPCN | 236 |
| Peptide-AI | YDCGPDVDESYVCV | 237 |
| Peptide-AJ | IDCWPDWEDDTFCY | 238 |
| Peptide-AK | YLCGPDGDETLACY | 239 |
| Peptide-AL | VDCGPDGDESILCY | 240 |

What is claimed is:

1. An isolated polypeptide or polypeptide complex according to Formula I:
A₂-A₁-L₁-P₁-H₁ (Formula I)
wherein:
A₁ comprises a first antigen recognizing molecule that binds to an effector cell antigen;
P₁ comprises a peptide that binds to A₁;
L₁ comprises a linking moiety that connects A₁ to P₁ and is a substrate for a tumor specific protease;
H₁ comprises a half-life extending molecule; and
A₂ comprises a second antigen recognizing molecule that binds to tumor-associated calcium signal transducer 2 (TROP2).

2. The isolated polypeptide or polypeptide complex of claim 1, wherein the first antigen recognizing molecule comprises an antibody or antibody fragment.

3. The isolated polypeptide or polypeptide complex of claim 1, wherein the first antigen recognizing molecule comprises an antibody or antibody fragment that is human or humanized.

4. The isolated polypeptide or polypeptide complex of claim 1, wherein L₁ is bound to N-terminus of the first antigen recognizing molecule.

5. The isolated polypeptide or polypeptide complex of claim 1, wherein A₂ is bound to C-terminus of the first antigen recognizing molecule.

6. The isolated polypeptide or polypeptide complex of claim 1, wherein L₁ is bound to C-terminus of the first antigen recognizing molecule.

7. The isolated polypeptide or polypeptide complex of claim 1, wherein A₂ is bound to N-terminus of the first antigen recognizing molecule.

8. The isolated polypeptide or polypeptide complex of claim 2, wherein the antibody or antibody fragment comprises a single chain variable fragment, a single domain antibody, or a Fab fragment.

9. The isolated polypeptide or polypeptide complex of claim 8, wherein A₁ is the single chain variable fragment (scFv).

10. The isolated polypeptide or polypeptide complex of claim 9, wherein the scFv comprises a scFv heavy chain polypeptide and a scFv light chain polypeptide.

11. The isolated polypeptide or polypeptide complex of claim 8, wherein A₁ is the single domain antibody.

12. The isolated polypeptide or polypeptide complex of claim 2, wherein the antibody or antibody fragment comprises a single chain variable fragment (scFv), a heavy chain variable domain (VH domain), a light chain variable domain (VL domain), or a variable domain (VHH) of a camelid derived single domain antibody.

13. The isolated polypeptide or polypeptide complex of claim 1, wherein A₁ comprises an anti-CD3e single chain variable fragment.

14. The isolated polypeptide or polypeptide complex of claim 1, wherein A₁ comprises an anti-CD3e single chain variable fragment that has a K_{D} binding of 1 μM or less to CD3 on CD3 expressing cells.

15. The isolated polypeptide or polypeptide complex of claim 1, wherein the effector cell antigen comprises CD3.

16. The isolated polypeptide or polypeptide complex of claim 1, wherein A₁ comprises a variable light chain and variable heavy chain each of which is capable of specifically binding to human CD3.

17. The isolated polypeptide or polypeptide complex of claim 1, wherein A₁ comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, 15865, 15865v12, 15865v16, and 15865v19.

18. The isolated polypeptide or polypeptide complex of claim 1, wherein the isolated polypeptide or polypeptide complex of Formula I binds to an effector cell when L₁ is cleaved by the tumor specific protease.

19. The isolated polypeptide or polypeptide complex of claim 1, wherein the isolated polypeptide or polypeptide complex of Formula I binds to an effector cell when L₁ is cleaved by the tumor specific protease and A₁ binds to the effector cell.

20. The isolated polypeptide or polypeptide complex of claim 18, wherein the effector cell is a T cell.

21. The isolated polypeptide or polypeptide complex of claim 18, wherein A₁ binds to a polypeptide that is part of a TCR-CD3 complex on the effector cell.

22. The isolated polypeptide or polypeptide complex of claim 21, wherein the polypeptide that is part of the TCR-CD3 complex is human CD3ε.

23. The isolated polypeptide or polypeptide complex of claim 9, wherein the effector cell antigen comprises CD3, wherein the scFv comprises complementarity determining regions (CDRs): HC-CDR1, HC-CDR2, and HC-CDR3, wherein the HC-CDR1, the HC-CDR2, and the HC-CDR3 of the scFv comprise: HC-CDR1: SEQ ID NO: 1, HC-CDR2: SEQ ID NO: 2, and HC-CDR3: SEQ ID NO: 3; and the scFv comprises CDRs: LC-CDR1, LC-CDR2, and LC-CDR3, wherein the LC-CDR1, the LC-CDR2, and the LC-CDR3 of the scFv comprise LC-CDR1: SEQ ID NO: 4, LC-CDR2: SEQ ID NO:5, and LC-CDR3: SEQ ID NO: 6.

24. The isolated polypeptide or polypeptide complex of claim 1, wherein the effector cell antigen comprises CD3, wherein A₁ comprises complementarity determining regions (CDRs): HC-CDR1, HC-CDR2, and HC-CDR3, wherein the HC-CDR1, the HC-CDR2, and the HC-CDR3 of A₁ comprise: HC-CDR1: SEQ ID NO: 1, HC-CDR2: SEQ ID NO: 2, and HC-CDR3: SEQ ID NO: 3; and A₁ comprises CDRs: LC-CDR1, LC-CDR2, and LC-CDR3, wherein the LC-CDR1, the LC-CDR2, and the LC-CDR3 of A₁ comprise LC-CDR1: SEQ ID NO: 4, LC-CDR2: SEQ ID NO:5, and LC-CDR3: SEQ ID NO: 6.

25. The isolated polypeptide or polypeptide complex of claim 9, wherein the effector cell antigen comprises CD3, and the scFv comprises an amino acid sequence according to SEQ ID NO: 13.

26. The isolated polypeptide or polypeptide complex of claim 9, wherein the effector cell antigen comprises CD3, wherein the scFv comprises complementarity determining regions (CDRs): HC-CDR1, HC-CDR2, and HC-CDR3, wherein the HC-CDR1, the HC-CDR2, and the HC-CDR3 of the scFv comprise: HC-CDR1: SEQ ID NO: 7, HC-CDR2: SEQ ID NO: 8, and HC-CDR3: SEQ ID NO: 9; and the scFv comprises CDRs: LC-CDR1, LC-CDR2, and LC-CDR3, wherein the LC-CDR1, the LC-CDR2, and the LC-CDR3 of the scFv comprise LC-CDR1: SEQ ID NO: 10, LC-CDR2: SEQ ID NO: 11, and LC-CDR3: SEQ ID NO: 12.

27. The isolated polypeptide or polypeptide complex of claim 1, wherein the effector cell antigen comprises CD3, wherein A₁ comprises complementarity determining regions (CDRs): HC-CDR1, HC-CDR2, and HC-CDR3, wherein the HC-CDR1, the HC-CDR2, and the HC-CDR3 of A₁ comprise: HC-CDR1: SEQ ID NO: 7, HC-CDR2: SEQ ID NO: 8, and HC-CDR3: SEQ ID NO: 9; and A₁ comprises CDRs: LC-CDR1, LC-CDR2, and LC-CDR3, wherein the LC-CDR1, the LC-CDR2, and the LC-CDR3 of A₁ comprise LC-CDR1: SEQ ID NO: 10, LC-CDR2: SEQ ID NO: 11, and LC-CDR3: SEQ ID NO: 12.

28. The isolated polypeptide or polypeptide complex of claim 9, wherein the effector cell antigen comprises CD3, and the scFv comprises an amino acid sequence according to SEQ ID NO: 14.

29. The isolated polypeptide or polypeptide complex of claim 10, wherein the second antigen recognizing molecule comprises an antibody or antibody fragment.

30. The isolated polypeptide or polypeptide complex of claim 29, wherein the antibody or antibody fragment thereof comprises a single chain variable fragment, a single domain antibody, or a Fab.

31. The isolated polypeptide or polypeptide complex of claim 30, wherein the antibody or antibody fragment thereof comprises a single chain variable fragment (scFv), a heavy chain variable domain (VH domain), a light chain variable domain (VL domain), a variable domain (VHH) of a camelid derived single domain antibody.

32. The isolated polypeptide or polypeptide complex of claim 31, wherein the antibody or antibody fragment thereof is humanized or human.

33. The isolated polypeptide or polypeptide complex of claim 30, wherein A₂ is the Fab.

34. The isolated polypeptide or polypeptide complex of claim 33, wherein the Fab comprises (a) a Fab light chain polypeptide and (b) a Fab heavy chain polypeptide.

35. The isolated polypeptide or polypeptide complex of claim 33, wherein the Fab comprises complementarity determining regions (CDRs): HC-CDR1, HC-CDR2, and HC-CDR3, wherein the HC-CDR1, the HC-CDR2, and the HC-CDR3 of the Fab comprise: HC-CDR1: SEQ ID NO: 15, HC-CDR2: SEQ ID NO: 16, and HC-CDR3: SEQ ID NO: 17; and the Fab comprises CDRs: LC-CDR1, LC-CDR2, and LC-CDR3, wherein the LC-CDR1, the LC-CDR2, and the LC-CDR3 of the Fab comprise LC-CDR1: SEQ ID NO: 18, LC-CDR2: SEQ ID NO: 19, and LC-CDR3: SEQ ID NO: 20.

36. The isolated polypeptide or polypeptide complex of claim 1, wherein A₂ comprises complementarity determining regions (CDRs): HC-CDR1, HC-CDR2, and HC-CDR3, wherein the HC-CDR1, the HC-CDR2, and the HC-CDR3 of A₂ comprise: HC-CDR1: SEQ ID NO: 15, HC-CDR2: SEQ ID NO: 16, and HC-CDR3: SEQ ID NO: 17; and A₂ comprises CDRs: LC-CDR1, LC-CDR2, and LC-CDR3, wherein the LC-CDR1, the LC-CDR2, and the LC-CDR3 of A₂ comprise LC-CDR1: SEQ ID NO: 18, LC-CDR2: SEQ ID NO: 19, and LC-CDR3: SEQ ID NO: 20.

37. The isolated polypeptide or polypeptide complex of claim 34, wherein the Fab light chain polypeptide comprises an amino acid sequence according to SEQ ID NO: 21.

38. The isolated polypeptide or polypeptide complex of claim 34, wherein Fab heavy chain polypeptide comprises an amino acid sequence according to SEQ ID NO: 22.

39. The isolated polypeptide or polypeptide complex of claim 34, wherein the Fab light chain polypeptide of A₂ is bound to a C-terminus of the single chain variable fragment (scFv) of A₁.

40. The isolated polypeptide or polypeptide complex of claim 34, wherein the Fab heavy chain polypeptide of A₂ is bound to a C-terminus of the single chain variable fragment (scFv) A₁.

41. The isolated polypeptide or polypeptide complex of claim 34, wherein the Fab light chain polypeptide of A₂ is bound to a N-terminus of the single chain variable fragment (scFv) of A₁.

42. The isolated polypeptide or polypeptide complex of claim 34, wherein the Fab heavy chain polypeptide of A₂ is bound to a N-terminus of the single chain variable fragment (scFv) A₁.

43. The isolated polypeptide or polypeptide complex of claim 34, wherein the Fab heavy chain polypeptide of A₂ is bound to the scFv heavy chain polypeptide of A₁.

44. The isolated polypeptide or polypeptide complex of claim 34, wherein the Fab light chain polypeptide of A₂ is bound to the scFv heavy chain polypeptide of A₁.

45. The isolated polypeptide or polypeptide complex of claim 34, wherein the Fab heavy chain polypeptide of A₂ is bound to the scFv light chain polypeptide of A₁.

46. The isolated polypeptide or polypeptide complex of claim 34, wherein the Fab light chain polypeptide of A₂ is bound to the scFv light chain polypeptide of A₁.

47. The isolated polypeptide or polypeptide complex of claim 34, wherein A₂ further comprises P₂ and L₂, wherein P₂ comprises a peptide that binds to A₂; and L₂ comprises a linking moiety that connects A₂ to P₂ and is a substrate for a tumor specific protease.

48. The isolated polypeptide or polypeptide complex of claim 47, wherein the isolated polypeptide or polypeptide complex is according to Formula Ia
P₂-L₂-A₂-A₁-L₁-P₁-H₁ (Formula Ia).

49. The isolated polypeptide or polypeptide complex of claim 48, wherein the Fab heavy chain polypeptide of A₂ is bound to the scFv heavy chain polypeptide of A₁ and L₂ is bound to the Fab light chain polypeptide of A₂.

50. The isolated polypeptide or polypeptide complex of claim 48, wherein the Fab light chain polypeptide of A₂ is bound to the scFv heavy chain polypeptide of A₁ and L₂ is bound to the Fab heavy chain polypeptide of A₂.

51. The isolated polypeptide or polypeptide complex of claim 48, wherein the Fab heavy chain polypeptide of A₂ is bound to the scFv light chain polypeptide of A₁ and L₂ is bound to the Fab light chain polypeptide of A₂.

52. The isolated polypeptide or polypeptide complex of claim 48, wherein the Fab light chain polypeptide of A₂ is bound to the scFv light chain polypeptide of A₁ and L₂ is bound to the Fab heavy chain polypeptide of A₂.

53. The isolated polypeptide or polypeptide complex of claim 1, wherein P₁ impairs binding of A₁ to the effector cell antigen.

54. The isolated polypeptide or polypeptide complex of claim 1, wherein P₁ is bound to A₁ through ionic interactions, electrostatic interactions, hydrophobic interactions, Pi-stacking interactions, and H-bonding interactions, or a combination thereof.

55. The isolated polypeptide or polypeptide complex of claim 1, wherein P₁ has less than 70% sequence homology to the effector cell antigen.

56. The isolated polypeptide or polypeptide complex of claim 48, wherein P₂ impairs binding of A₂ to TROP2.

57. The isolated polypeptide or polypeptide complex of claim 48, wherein P₂ is bound to A₂ through ionic interactions, electrostatic interactions, hydrophobic interactions, Pi-stacking interactions, and H-bonding interactions, or a combination thereof.

58. The isolated polypeptide or polypeptide complex of claim 48, wherein P₂ is bound to A₂ at or near an antigen binding site.

59. The isolated polypeptide or polypeptide complex of claim 48, wherein P₂ has less than 70% sequence homology to TROP2.

60. The isolated polypeptide or polypeptide complex of claim 48, wherein P₁ or P₂ comprises a peptide sequence of at least 10 amino acids in length.

61. The isolated polypeptide or polypeptide complex of claim 48, wherein P₁ or P₂ comprises a peptide sequence of at least 10 amino acids in length and no more than 20 amino acids in length.

62. The isolated polypeptide or polypeptide complex of claim 48, wherein P₁ or P₂ comprises a peptide sequence of at least 16 amino acids in length.

63. The isolated polypeptide or polypeptide complex of claim 48, wherein P₁ or P₂ comprises a peptide sequence of no more than 40 amino acids in length.

64. The isolated polypeptide or polypeptide complex of claim 48, wherein P₁ or P₂ comprises at least two cysteine amino acid residues.

65. The isolated polypeptide or polypeptide complex of claim 48, wherein P₁ or P₂ comprises a cyclic peptide or a linear peptide.

66. The isolated polypeptide or polypeptide complex of claim 48, wherein P₁ or P₂ comprises a cyclic peptide.

67. The isolated polypeptide or polypeptide complex of claim 48, wherein P₁ or P₂ comprises a linear peptide.

68. The isolated polypeptide or polypeptide complex of claim 1, wherein P₁ comprises at least two cysteine amino acid residues.

69. The isolated polypeptide or polypeptide complex of claim 1, wherein P₁ comprises an amino acid sequence according to SEQ ID NO: 26, 27, or 122.

70. The isolated polypeptide or polypeptide complex of claim 48, wherein P₂ comprises an amino acid sequence according to any one of SEQ ID NOs: 23-25.

71. The isolated polypeptide or polypeptide complex of claim 48, wherein P₂ comprises an amino acid sequence according to X₁-X₂-X₃-X₄-C-X₆-X₇-X₈-X₉-X₁₀-C-X₁₂-X₁₃-X₁₄ and
X₁ is selected from N, D, S, Y, A, F, H, T, L, and V;
X₂ is selected from S, T, D, A, H, V, Y, N, F, I, and L;
X₃ is selected from L, I, and V;
X₄ is selected from F, L, V, M, W, I, Y, and H;
X₆ is selected from V, F, L, I, and W;
X₇ is selected from K, R, Q, N, H, and M;
X₈ is selected from N and K;
X₉ is selected from L, V, and I;
X₁₀ is selected from Y, W, F, Q, and L;
X₁₂ is selected from W and V;
X₁₃ is selected from I, N, H, T, V, Y, and D;
X₁₄ is selected from D, V, A, S, I, T, N, Y, H, and P.

72. The isolated polypeptide or polypeptide complex of claim 71, wherein
X₁ is selected from N, D, S, Y, A, F, and T;
X₂ is selected from S, T, D, A, H, V, Y, and N;
X₃ is L;
X₄ is selected from F, L, V, M, and W;
X₆ is selected from V, F, and L;
X₇ is selected from K, R, Q, and N;
X₈ is selected from N and K;
X₉ is selected from L, V, and I;
X₁₀ is selected from Y, W, and F;
X₁₂ is W;
X₁₃ is selected from I, N, H, and T;
X₁₄ is selected from D, V, A, S, I, T, and N.

73. The isolated polypeptide or polypeptide complex of claim 72, wherein
X₁ is selected from N, D, S, and Y;
X₂ is selected from S, T, and D;
X₃ is L;
X₄ is selected from F, L, and V;
X₆ is selected from V and F;
X₇ is selected from K, R, and Q;
X₈ is N;
X₉ is selected from L and V;
X₁₀ is selected from Y and W;
X₁₂ is W;
X₁₃ is selected from I, N, and H;
X₁₄ is selected from D, V, A, and S.

74. The isolated polypeptide or polypeptide complex of claim 48, wherein P₂ comprises an amino acid sequence according to J₁-J₂-J₃-C-J₅-J₆-J₇-J₈-W-J₁₀-J₁₁-C-J₁₃-J₁₄ and
J₁ is selected from V, I, L, P, E, F, and M;
J₂ is selected from D and N;
J₃ is selected from F and W;
J₅ is selected from A, E, S, R, K, Y, L, Q, G, M, F, T, W, and D;
J₆ is selected from L, M, I, V, F, T, R, and S;
J₇ is selected from Y, F, and N;
J₈ is selected from N, R, D, H, K, Q, S, G, A, E, and M;
J₁₀ is selected from P and R;
J₁₁ is selected from V and I;
J₁₃ is selected from D, G, N, R, S, Q, Y, T, A, E, L, V, K, M, I, H, F, and W;
J₁₄ is selected from T, S, Q, L, D, N, A, E, K, M, V, R, I, H, P, V, and W; or
P₂ comprises an amino acid sequence according to B₁-B₂-B₃-C-B₅-B₆-B₇-B₈-W-B₁₀-B₁₁-C-B₁₃-B₁₄ and
B₁ is selected from V, I, L, P, E, F, and M;
B₂ is selected from D and N;
B₃ is selected from F and W;
B₅ is selected from A, E, S, R, K, Y, L, Q, G, M, F, T, W, and D;
B₆ is selected from L, M, I, V, F, T, R, and S;
B₇ is selected from Y, F, and N;
B₈ is selected from N, R, D, H, K, Q, S, G, A, E, and M;
B₁₀ is selected from P and R;
B₁₁ is selected from V and I;
B₁₃ is selected from D, G, N, R, S, Q, Y, T, A, E, L, V, K, M, I, H, F, and W;
B₁₄ is selected from T, S, Q, L, D, N, A, E, K, M, V, R, I, H, P, V, G, and W.

75. The isolated polypeptide or polypeptide complex of claim 74, wherein
J₁ is selected from V, I, and L;
J₂ is D;
J₃ is F;
J₅ is selected from A, E, S, R, K, Y, and L;
J₆ is selected from L, M, and I;
J₇ is Y;
J₈ is selected from N, R, D, H, K, Q, S, and G;
J₁₀ is P;
J₁₁ is selected from V and I;
J₁₃ is selected from D, G, N, R, S, Q, Y, T, and A;
J₁₄ is selected from T, S, Q, L, D, N, A, and E; and
B₁ is selected from V, I, and L;
B₂ is D;
B₃ is F;
B₅ is selected from A, E, S, R, K, Y, M, G, and L;
B₆ is selected from L, M, and I;
B₇ is Y;
B₈ is selected from N, R, D, H, K, Q, S, and G;
B₁₀ is P;
B₁₁ is selected from V and I;
B₁₃ is selected from D, G, N, R, S, Q, Y, T, H, and A;
B₁₄ is selected from T, S, Q, L, D, N, A, G, and E.

76. The isolated polypeptide or polypeptide complex of claim 75, wherein
J₁ is selected from V, I, and L;
J₂ is D;
J₃ is F;
J₅ is selected from A, E, S, and R;
J₆ is selected from L, M, and I;
J₇ is Y;
J₈ is selected from N, R, and D;
J₁₀ is P;
J₁ is selected from V and I;
J₁₃ is selected from D, G, N, R, S, and Q;
J₁₄ is selected from T, S, Q, and L; and
B₁ is selected from V, I, and L;
B₂ is D;
B₃ is F;
B₅ is selected from A, E, S, K, M, G, and R;
B₆ is selected from L, M, and I;
B₇ is Y;
B₈ is selected from N, R, S, H, and D;
B₁₀ is P;
B₁₁ is selected from V and I;
B₁₃ is selected from D, G, N, R, S, H, A, Y, and Q;
B₁₄ is selected from T, S, Q, G, and L.

77. The isolated polypeptide or polypeptide complex of claim 48, wherein P₂ comprises the amino acid sequences according to SEQ ID NOs: 133-145.

78. The isolated polypeptide or polypeptide complex of claim 48, wherein P₂ comprises the amino acid sequences according to SEQ ID NOs: 146-158.

79. The isolated polypeptide or polypeptide complex of claim 48, wherein P₂ comprises an amino acid sequence according to any of the sequences of Table 27.

80. The isolated polypeptide or polypeptide complex of claim 48, wherein P₂ comprises the amino acid sequences according to SEQ ID NOs: 159-178.

81. The isolated polypeptide or polypeptide complex of claim 48, wherein P₂ comprises an amino acid sequence according to any of the sequences of Table 29.

82. The isolated polypeptide or polypeptide complex of claim 48, wherein P₂ comprises the amino acid sequences according to SEQ ID NOs: 179-201.

83. The isolated polypeptide or polypeptide complex of claim 48, wherein P₂ comprises the amino acid sequence according to SEQ ID NO: 24 or a peptide sequence that has 1, 2, or 3, amino acid substitutions, additions, or deletions relative to the amino acid sequence of SEQ ID NO: 24.

84. The isolated polypeptide or polypeptide complex of claim 48, wherein P₂ comprises the amino acid sequence according to SEQ ID NO: 181 or a peptide sequence that has 1, 2, or 3, amino acid substitutions, additions, or deletions relative to the amino acid sequence of SEQ ID NO: 181.

85. The isolated polypeptide or polypeptide complex of claim 48, wherein P₂ comprises the amino acid sequence according to SEQ ID NO: 186 or a peptide sequence that has 1, 2, or 3, amino acid substitutions, additions, or deletions relative to the amino acid sequence of SEQ ID NO: 186.

86. The isolated polypeptide or polypeptide complex of claim 48, wherein P₂ comprises the amino acid sequence according to SEQ ID NO: 24.

87. The isolated polypeptide or polypeptide complex of claim 48, wherein P₂ comprises the amino acid sequence according to SEQ ID NO: 181.

88. The isolated polypeptide or polypeptide complex of claim 48, wherein P₂ comprises the amino acid sequence according to SEQ ID NO: 186.

89. The isolated polypeptide or polypeptide complex of claim 1, wherein P₁ comprises an amino acid sequence according to Z₁-Z₂-C-Z₄-P-Z₆-Z₇-Z₈-Z₉-Z₁₀-Z₁₁-Z₁₂-C-Z₁₄ and
Z₁ is selected from D, Y, F, I, N, V, H, L, A, T, S, and P;
Z₂ is selected from D, Y, L, F, I, N, A, V, H, T, and S;
Z₄ is selected from G and W;
Z₆ is selected from E, D, V, and P;
Z₇ is selected from W, L, F, V, G, M, I, and Y;
Z₈ is selected from E, D, P, and Q;
Z₉ is selected from E, D, Y, V, F, W, P, L, and Q;
Z₁₀ is selected from S, D, Y, T, I, F, V, N, A, P, L, and H;
Z₁₁ is selected from I, Y, F, V, L, T, N, S, D, A, and H;
Z₁₂ is selected from F, D, Y, L, I, V, A, N, T, P, S, and H;
Z₁₄ is selected from D, Y, N, F, I, P, V, A, T, H, L and S; or
P₁ comprises an amino acid sequence according to U₁-U₂-C-U₄-P-U₆-U₇-U₈-U₉-U₁₀-U₁₁-U₁₂-C-U₁₄ and
U₁ is selected from D, Y, F, I, N, V, H, L, A, T, S, and P;
U₂ is selected from D, Y, L, F, I, N, A, V, H, T, and S;
U₄ is selected from G and W;
U₆ is selected from E, D, V, and P;
U₇ is selected from W, L, F, V, G, M, I, and Y;
U₈ is selected from E, D, P, and Q;
U₉ is selected from E, D, Y, V, F, W, P, L, and Q;
U₁₀ is selected from S, D, Y, T, I, F, V, N, A, P, L, and H;
U₁n is selected from I, Y, F, V, L, T, N, S, D, A, and H;
U₁₂ is selected from F, D, Y, L, I, V, A, N, T, P, S, G, and H;
U₁₄ is selected from D, Y, N, F, I, P, V, A, T, H, L, M, and S.

90. The isolated polypeptide or polypeptide complex of claim 89, wherein
Z₁ is selected from D, Y, F, I, and N;
Z₂ is selected from D, Y, L, F, I, and N;
Z₄ is selected from G and W;
Z₆ is selected from E and D;
Z₇ is selected from W, L, F, and V;
Z₈ is selected from E and D;
Z₉ is selected from E, D, Y, and V;
Z₁₀ is selected from S, D, Y, T, and I;
Z₁₁ is selected from I, Y, F, V, L, and T;
Z₁₂ is selected from F, D, Y, L, I, V, A, and N;
Z₁₄ is selected from D, Y, N, F, I, and P; and
U₁ is selected from D, Y, F, I, V, and N;
U₂ is selected from D, Y, L, F, I, and N;
U₄ is selected from G and W;
U₆ is selected from E and D;
U₇ is selected from W, L, F, G, and V;
U₈ is selected from E and D;
U₉ is selected from E, D, Y, and V;
U₁₀ is selected from S, D, Y, T, and I;
U₁₁ is selected from I, Y, F, V, L, and T;
U₁₂ is selected from F, D, Y, L, I, V, A, G, and N;
U₁₄ is selected from D, Y, N, F, I, M, and P.

91. The isolated polypeptide or polypeptide complex of claim 90, wherein
Z₁ is selected from D, Y, and F;
Z₂ is selected from D, Y, L, and F;
Z₄ is selected from G and W;
Z₆ is selected from E and D;
Z₇ is selected from W, L, and F;
Z₈ is selected from E and D;
Z₉ is selected from E and D;
Z₁₀ is selected from S, D, and Y;
Z₁₁ is selected from I, Y, and F;
Z₁₂ is selected from F, D, Y, and L;
Z₁₄ is selected from D, Y, and N; and
U₁ is selected from D, Y, V, and F;
U₂ is selected from D, Y, L, and F;
U₄ is selected from G and W;
U₆ is selected from E and D;
U₇ is selected from W, L, G, and F;
U₈ is selected from E and D;
U₉ is selected from E and D;
U₁₀ is selected from S, D, T, and Y;
Uₙ is selected from I, Y, V, L, and F;
U₁₂ is selected from F, D, Y, G, A, and L;
U₁₄ is selected from D, Y, M, and N.

92. The isolated polypeptide or polypeptide complex of claim 1, wherein P₁ comprises the amino acid sequences according to SEQ ID NOs: 202-228.

93. The isolated polypeptide or polypeptide complex of claim 1, wherein P₁ comprises an amino acid sequences according to any of the sequences of Table 35.

94. The isolated polypeptide or polypeptide complex of claim 1, wherein P₁ comprises the amino acid sequences according to SEQ ID NOs: 229-240.

95. The isolated polypeptide or polypeptide complex of claim 1, wherein P₁ comprises the amino acid sequence according to SEQ ID NO: 239 or a peptide sequence that has 1, 2, or 3, amino acid substitutions, additions, or deletions relative to the amino acid sequence of SEQ ID NO: 239.

96. The isolated polypeptide or polypeptide complex of claim 1, wherein P₁ comprises the amino acid sequence according to SEQ ID NO: 27 or a peptide sequence that has 1, 2, or 3, amino acid substitutions, additions, or deletions relative to the amino acid sequence of SEQ ID NO: 27.

97. The isolated polypeptide or polypeptide complex of claim 1, wherein P₁ comprises the amino acid sequence according to SEQ ID NO: 26 or a peptide sequence that has 1, 2, or 3, amino acid substitutions, additions, or deletions relative to the amino acid sequence of SEQ ID NO: 26.

98. The isolated polypeptide or polypeptide complex of claim 1, wherein P₁ comprises the amino acid sequence according to SEQ ID NO: 239.

99. The isolated polypeptide or polypeptide complex of claim 1, wherein P₁ comprises the amino acid sequence according to SEQ ID NO: 27.

100. The isolated polypeptide or polypeptide complex of claim 1, wherein P₁ comprises the amino acid sequence according to SEQ ID NO: 26.

101. The isolated polypeptide or polypeptide complex of claim 1, wherein L₁ is bound to N-terminus of A₁.

102. The isolated polypeptide or polypeptide complex of claim 1, wherein L₁ is bound to C-terminus of A₁.

103. The isolated polypeptide or polypeptide complex of claim 48, wherein L₂ is bound to N-terminus of A₂.

104. The isolated polypeptide or polypeptide complex of claim 48, wherein L₂ is bound to C-terminus of A₂.

105. The isolated polypeptide or polypeptide complex of claim 48, wherein L₁ or L₂ is a peptide sequence having at least 5 to no more than 50 amino acids.

106. The isolated polypeptide or polypeptide complex of claim 48, wherein L₁ or L₂ is a peptide sequence having at least 10 to no more than 30 amino acids.

107. The isolated polypeptide or polypeptide complex of claim 48, wherein L₁ or L₂ is a peptide sequence having at least 10 amino acids.

108. The isolated polypeptide or polypeptide complex of claim 48, wherein L₁ or L₂ is a peptide sequence having at least 18 amino acids.

109. The isolated polypeptide or polypeptide complex of claim 48, wherein L₁ or L₂ is a peptide sequence having at least 26 amino acids.

110. The isolated polypeptide or polypeptide complex of claim 48, wherein L₁ or L₂ has a formula comprising (G₂S)ₙ (SEQ ID NO: 243), wherein n is an integer from 1 to 3.

111. The isolated polypeptide or polypeptide complex of claim 1, wherein L₁ has a formula selected from the group consisting of (G₂S)ₙ, (GS)ₙ, (GSGGS)ₙ (SEQ ID NO: 62), (GGGS)ₙ (SEQ ID NO: 63), (GGGGS)ₙ (SEQ ID NO: 64), and (GSSGGS)ₙ (SEQ ID NO: 65), wherein n is an integer of at least 1.

112. The isolated polypeptide or polypeptide complex of claim 1, wherein P₁ becomes unbound from A₁ when L₁ is cleaved by the tumor specific protease thereby exposing A₁ to the effector cell antigen.

113. The isolated polypeptide or polypeptide complex of claim 48, wherein P₂ becomes unbound from A₂ when L₂ is cleaved by the tumor specific protease thereby exposing A₂ to TROP2.

114. The isolated polypeptide or polypeptide complex of claim 1, wherein the tumor specific protease is selected from the group consisting of a matrix metalloprotease (MMP), serine protease, cysteine protease, threonine protease, and aspartic protease.

115. The isolated polypeptide or polypeptide complex of claim 114, wherein the matrix metalloprotease comprises MMP2, MMP7, MMP9, MMP13, or MMP14.

116. The isolated polypeptide or polypeptide complex of claim 114, wherein the serine protease comprises matriptase (MTSP1), urokinase, or hepsin.

117. The isolated polypeptide or polypeptide complex of claim 48, wherein L₁ or L₂ comprises a urokinase cleavable amino acid sequence, a matriptase cleavable amino acid sequence, a matrix metalloprotease cleavable amino acid sequence, or a legumain cleavable amino acid sequence.

118. The isolated polypeptide or polypeptide complex of claim 48, wherein L₁ or L₂ comprises an amino acid sequence according to SEQ ID NO: 31 or 32.

119. The isolated polypeptide or polypeptide complex of claim 48, wherein L₁ or L₂ comprises an amino acid sequence according to SEQ ID NO: 58 or 59.

120. The isolated polypeptide or polypeptide complex of claim 48, wherein L₁ or L₂ comprises an amino acid sequence according to any one of SEQ ID NOs: 28-61.

121. The isolated polypeptide or polypeptide complex of claim 48, wherein L₁ or L₂ comprises an amino acid sequence of Linker 25 (ISSGLLSGRSDAG) (SEQ ID NO: 54), Linker 26 (AAGLLAPPGGLSGRSDAG) (SEQ ID NO: 55), Linker 27 (SPLGLSGRSDAG) (SEQ ID NO: 56), or Linker 28 (LSGRSDAGSPLGLAG) (SEQ ID NO: 57), or an amino acid sequence that has 1, 2, or 3 amino acid substitutions, additions, or deletions relative to the amino acid sequence of Linker 25, Linker 26, Linker 27, or Linker 28.

122. The isolated polypeptide or polypeptide complex of claim 1, wherein H₁ comprises a polymer.

123. The isolated polypeptide or polypeptide complex of claim 122, wherein the polymer is polyethylene glycol (PEG).

124. The isolated polypeptide or polypeptide complex of claim 1, wherein H₁ comprises albumin.

125. The isolated polypeptide or polypeptide complex of claim 1, wherein H₁ comprises an Fc domain.

126. The isolated polypeptide or polypeptide complex of claim 124, wherein the albumin is serum albumin.

127. The isolated polypeptide or polypeptide complex of claim 124, wherein the albumin is human serum albumin.

128. The isolated polypeptide or polypeptide complex of claim 1, wherein H₁ comprises a polypeptide, a ligand, or a small molecule.

129. The isolated polypeptide or polypeptide complex of claim 128, wherein the polypeptide, the ligand or the small molecule binds serum protein or a fragment thereof, a circulating immunoglobulin or a fragment thereof, or CD35/CR1.

130. The isolated polypeptide or polypeptide complex of claim 129, wherein the serum protein comprises a thyroxine-binding protein, a transthyretin, a 1-acid glycoprotein, a transferrin, transferrin receptor or a transferrin-binding portion thereof, a fibrinogen, or an albumin.

131. The isolated polypeptide or polypeptide complex of claim 129, wherein the circulating immunoglobulin molecule comprises IgG1, IgG2, IgG3, IgG4, slgA, IgM or IgD.

132. The isolated polypeptide or polypeptide complex of claim 129, wherein the serum protein is albumin.

133. The isolated polypeptide or polypeptide complex of claim 128, wherein the polypeptide is an antibody.

134. The isolated polypeptide or polypeptide complex of claim 133, wherein the antibody comprises a single domain antibody, a single chain variable fragment, or a Fab.

135. The isolated polypeptide or polypeptide complex of claim 134, wherein the single domain antibody comprises a single domain antibody that binds to albumin.

136. The isolated polypeptide or polypeptide complex of claim 134, wherein the single domain antibody is a human or humanized antibody.

137. The isolated polypeptide or polypeptide complex of claim 134, wherein the single domain antibody is 645gH1gL1.

138. The isolated polypeptide or polypeptide complex of claim 134, wherein the single domain antibody is 645dsgH5gL4.

139. The isolated polypeptide or polypeptide complex of claim 134, wherein the single domain antibody is 23-13-A01-sc02.

140. The isolated polypeptide or polypeptide complex of claim 134, wherein the single domain antibody is A10m3 or a fragment thereof.

141. The isolated polypeptide or polypeptide complex of claim 134, wherein the single domain antibody is DOM7r-31.

142. The isolated polypeptide or polypeptide complex of claim 134, wherein the single domain antibody is DOM7h-11-15.

143. The isolated polypeptide or polypeptide complex of claim 134, wherein the single domain antibody is Alb-1, Alb-8, or Alb-23.

144. The isolated polypeptide or polypeptide complex of claim 134, wherein the single domain antibody is 10E.

145. The isolated polypeptide or polypeptide complex of claim 134, wherein the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 66, HC-CDR2: SEQ ID NO: 67, and HC-CDR3: SEQ ID NO: 68.

146. The isolated polypeptide or polypeptide complex of claim 134, wherein the single domain antibody comprises an amino acid sequence according to SEQ ID NO: 69.

147. The isolated polypeptide or polypeptide complex of claim 134, wherein the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 70, HC-CDR2: SEQ ID NO: 71, and HC-CDR3: SEQ ID NO: 72.

148. The isolated polypeptide or polypeptide complex of claim 134, wherein the single domain antibody comprises an amino acid sequence according to SEQ ID NO: 73.

149. The isolated polypeptide or polypeptide complex of claim 134, wherein the single domain antibody is SA21.

150. The isolated polypeptide or polypeptide complex of claim 1, wherein the isolated polypeptide or polypeptide complex comprises a modified amino acid, a non-natural amino acid, a modified non-natural amino acid, or a combination thereof.

151. The isolated polypeptide or polypeptide complex of claim 150, wherein the modified amino acid or modified non-natural amino acid comprises a post-translational modification.

152. The isolated polypeptide or polypeptide complex of claim 1, wherein H₁ comprises a linking moiety (L₃) that connects H₁ to P₁.

153. The isolated polypeptide or polypeptide complex of claim 152, wherein L₃ is a peptide sequence having at least 5 to no more than 50 amino acids.

154. The isolated polypeptide or polypeptide complex of claim 152, wherein L₃ is a peptide sequence having at least 10 to no more than 30 amino acids.

155. The isolated polypeptide or polypeptide complex of claim 152, wherein L₃ is a peptide sequence having at least 10 amino acids.

156. The isolated polypeptide or polypeptide complex of claim 152, wherein L₃ is a peptide sequence having at least 18 amino acids.

157. The isolated polypeptide or polypeptide complex of claim 152, wherein L₃ is a peptide sequence having at least 26 amino acids.

158. The isolated polypeptide or polypeptide complex of claim 152, wherein L₃ has a formula selected from the group consisting of (G₂S)ₙ, (GS)ₙ, (GSGGS)ₙ (SEQ ID NO: 62), (GGGS)ₙ (SEQ ID NO: 63), (GGGGS)ₙ (SEQ ID NO: 64), and (GSSGGS)ₙ (SEQ ID NO: 65), wherein n is an integer of at least 1.

159. The isolated polypeptide or polypeptide complex of claim 152, wherein L₃ comprises an amino acid sequence according to SEQ ID NO: 30.

160. The isolated polypeptide or polypeptide complex of claim 1, wherein the isolated polypeptide or polypeptide complex comprises an amino acid sequence with at least 95% sequence identity to SEQ ID NOs: 74-132, 241-242.

161. A pharmaceutical composition comprising: (a) the isolated polypeptide or polypeptide complex of claim 1; and (b) a pharmaceutically acceptable excipient.

162. An isolated recombinant nucleic acid molecule encoding the isolated polypeptide or polypeptide complex of claim 1.
